(19) United States
(12) Patent Application Publication
(10) Pub. No.: US 2024/0180027 A1
(43) Pub. Date: May 30, 2024

(54) ORGANIC LIGHT EMITTING DEVICE, AND COMPOSITION FOR FORMATION OF ORGANIC MATERIAL LAYER

(71) Applicant: LT MATERIALS CO., LTD., Yongin-City (KR)

(72) Inventors: Sol LEE, Yongin-City (KR); Jun-Tae MO, Yongin-City (KR); Dong-Jun KIM, Yongin-City (KR); Dae-Hyuk CHOI, Yongin-City (KR)

(73) Assignee: LT MATERIALS CO., LTD., Yongin-City (KR)

(21) Appl. No.: 18/373,467

(22) Filed: Sep. 27, 2023

(30) Foreign Application Priority Data
Oct. 4, 2022 (KR) 10-2022-0126629

(51) Int. Cl. H10K 85/60; C07D 405/04; C07D 405/10; C07D 405/14; C07D 409/04; C07D 409/14; C07D 487/04; C07D 491/048; C09K 11/02

(52) U.S. Cl. CPC H10K 85/654; C07D 405/04; C07D 405/10; C07D 405/14; C07D 409/04; C07D 409/14; C07D 487/04; C07D 491/048; C09K 11/02; H10K 85/615; H10K 85/626; H10K 85/633; H10K 85/636; H10K 85/657; H10K 85/6572; H10K 85/6574; H10K 85/6576; H10K 50/11

(57) ABSTRACT

Disclosed is an organic light emitting device and a composition for forming an organic material layer of the organic light emitting device.

### CROSS-REFERENCE TO RELATED APPLICATIONS

This application claims priority to and the benefit of Korean Patent Application No. 10-2022-0126629 filed in the Korean Intellectual Property Office on Oct. 4, 2022, the entire contents of which are incorporated herein by reference.

### TECHNICAL FIELD

The present specification relates to an organic light emitting device and a composition for forming an organic material layer.

### BACKGROUND ART

An electroluminescence device is a kind of self-emitting type display device, and has an advantage in that the viewing angle is wide, the contrast is excellent, and the response speed is fast.

An organic light emitting device is composed of a structure in which an organic thin film is disposed between two electrodes. When a voltage is applied to an organic light emitting device having such a structure, electrons and holes injected from the two electrodes are combined with each other in the organic thin film to make a pair, and then, the paired electrons and holes emit light while being annihilated. The organic thin film may be composed of a single layer or multiple layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may perform a function such as hole injection, hole transport, electron blocking, hole blocking, electron transport or electron injection.

In order to improve the performance, efficiency and service life of the organic light emitting device, there is a continuous need for developing a material for an organic thin film.

### RELATED ART DOCUMENTS

### Patent Documents

(Patent Document 1) U.S. Pat. No. 4,356,429

### SUMMARY OF THE INVENTION

The present specification has been made in an effort to provide an organic light emitting device and a composition for forming an organic material layer.

An exemplary embodiment of the present invention provides an organic light emitting device including: a first electrode; a second electrode; and an organic material layer provided between the first electrode and the second electrode, in which the organic material layer includes a compound of the following Chemical Formula 1 and a compound of the following Chemical Formula 2.

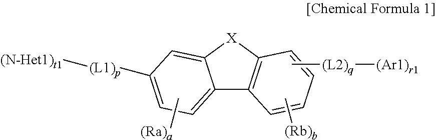

X is O or S,

Ar1 is a substituted or unsubstituted C6 to C60 aryl group; or a substituted or unsubstituted C2 to C60 heteroaryl group,

N-Het1 is a C2 to C60 heteroaryl group which is substituted or unsubstituted and includes N,

t1 and r1 are each an integer from 1 to 3, and when t1 is 2 or higher, N-Het1's are the same as or different from each other, and when r1 is 2 or higher, Ar1's are the same as or different from each other,

L1 and L2 are the same as or different from each other, and are each independently a direct bond; a substituted or unsubstituted C6 to C60 arylene group; or a substituted or unsubstituted C2 to C60 heteroarylene group,

p and q are each an integer from 0 to 3, and when p is 2 or higher, L1's are the same as or different from each other, and when q is 2 or higher, L2's are the same as or different from each other,

Ra and Rb are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and

a and b are each an integer from 0 to 3, and when a is 2 or higher, Ra's are the same as or different from each other, and when b is 2 or higher, Rb's are the same as or different from each other,

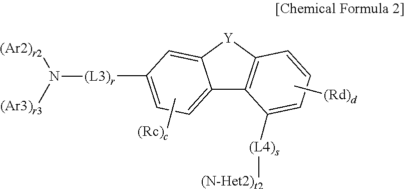

Y is O or S,

Ar2 and Ar3 are the same as or different from each other, and are each independently a substituted or unsubstituted C6 to C60 aryl group; or a substituted or unsubstituted C2 to C60 heteroaryl group,

N-Het2 is a C2 to C60 heteroaryl group which is substituted or unsubstituted and includes N,

t2, r2 and r3 are each an integer from 1 to 3, and when t2 is 2 or higher, N-Het2's are the same as or different from each other, and when r2 is 2 or higher, Ar2's are the same as or different from each other, and when r3 is 2 or higher, Ar3's are the same as or different from each other,

L3 and L4 are the same as or different from each other, and are each independently a direct bond; a substituted or unsubstituted C6 to C60 arylene group; or a substituted or unsubstituted C2 to C60 heteroarylene group,

r and s are each an integer from 0 to 3, and when r is 2 or higher, L3's are the same as or different from each other, and when s is 2 or higher, L4's are the same as or different from each other,

Rc and Rd are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and

c and d are each an integer from 0 to 3, and when c is 2 or higher, Rc's are the same as or different from each other, and when d is 2 or higher, Rd's are the same as or different from each other.

Another exemplary embodiment provides a composition for forming an organic material layer including the compound of Chemical Formula 1 and the compound of Chemical Formula 2.

The organic light emitting device of the present specification includes an organic material layer including a compound of Chemical Formula 1 and a compound of Chemical Formula 2, and the organic material layer including the compounds may be a hole injection layer, a hole transport layer, a light emitting layer, an electron transport layer, an electron injection layer, a charge generation layer, and the like. In particular, the organic material layer including the compounds may be a light emitting layer of an organic light emitting device.

When the compound of Chemical Formula 1 and the compound of Chemical Formula 2 are together used as materials for the light emitting layer of the organic light emitting device, the driving voltage of the device can be lowered, the light emitting efficiency can be improved, and the service life characteristics can be improved.

Specifically, by using both a compound of Chemical Formula 1, which includes a heteroaryl group including N and an aryl group or a heteroaryl group and a compound of Chemical Formula 2, which has a tricyclic hetero ring as a core structure and has an amine group at the third position of the core structure and an N-containing heteroaryl group at the ninth position, as host materials for a light emitting layer, an exciplex phenomenon occurs, so that driving voltage, light emitting efficiency and service life characteristics are improved.

### BRIEF DESCRIPTION OF THE DRAWINGS

FIGS. 1 to 4 are views each schematically illustrating a stacking structure of an organic light emitting device according to an exemplary embodiment of the present application.

### DETAILED DESCRIPTION

Hereinafter, the present specification will be described in more detail.

When one part “includes” one constituent element in the present specification, unless otherwise specifically described, this does not mean that another constituent element is excluded, but means that another constituent element may be further included.

In the present specification,

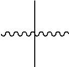

of a chemical formula means a position to which a constituent element is bonded.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, “substituted or unsubstituted” means being unsubstituted or substituted with one or more substituents selected from the group consisting of deuterium; a halogen group; —CN; a C1 to C60 alkyl group; a C2 to C60 alkenyl group; a C2 to C60 alkynyl group; a C1 to C60 haloalkyl group; a C1 to C60 alkoxy group; a C6 to C60 aryloxy group; a C1 to C60 alkylthioxy group; a C6 to C60 arylthioxy group; a C1 to C60 alkylsulfoxy group; a C6 to C60 arylsulfoxy group; a C3 to C60 cycloalkyl group; a C2 to C60 heterocycloalkyl group; a C6 to C60 aryl group; a C2 to C60 heteroaryl group; —SiRR′R″; —P(═O)RR′; and —NRR′, or a substituent to which two or more substituents selected among the exemplified substituents are linked, and R, R′ and R″ are each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group.

In the present specification, “when a substituent is not indicated in the structure of a chemical formula or compound” means that a hydrogen atom is bonded to a carbon atom. However, since deuterium (²H) is an isotope of hydrogen, some hydrogen atoms may be deuterium.

In an exemplary embodiment of the present application, “when a substituent is not indicated in the structure of a chemical formula or compound” may mean that all the positions that may be reached by the substituent are hydrogen or deuterium. That is, deuterium is an isotope of hydrogen, and some hydrogen atoms may be deuterium which is an isotope, and in this case, the content of deuterium may be 0% to 100%.

In an exemplary embodiment of the present application, in “the case where a substituent is not indicated in the structure of a chemical formula or compound”, when the content of deuterium is 0%, the content of hydrogen is 100%, and all the substituents do not explicitly exclude deuterium such as hydrogen, hydrogen and deuterium may be mixed and used in the compound.

In an exemplary embodiment of the present application, deuterium is one of the isotopes of hydrogen, is an element that has a deuteron composed of one proton and one neutron as a nucleus, and may be represented by hydrogen-2, and the element symbol may also be expressed as D or ²H.

In an exemplary embodiment of the present application, the isotope means an atom with the same atomic number (Z), but different mass numbers (A), and the isotope may also be interpreted as an element which has the same number of protons, but different number of neutrons.

In an exemplary embodiment of the present application, when the total number of substituents of a basic compound is defined as T1 and the number of specific substituents among the substituents is defined as T2, the content T % of the specific substituent may be defined as T2/T1×100=T %.

That is, in an example, a deuterium content of 20%

in a phenyl group represented by

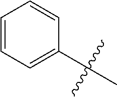 [SMILES: *c1ccccc1]

may be represented by 20% when the total number of substituents that the phenyl group can have is 5 (T1 in the formula) and the number of deuteriums among the substituents is 1 (T2 in the formula). That is, a deuterium content of 20% in the phenyl group may be represented by the following structural formula.

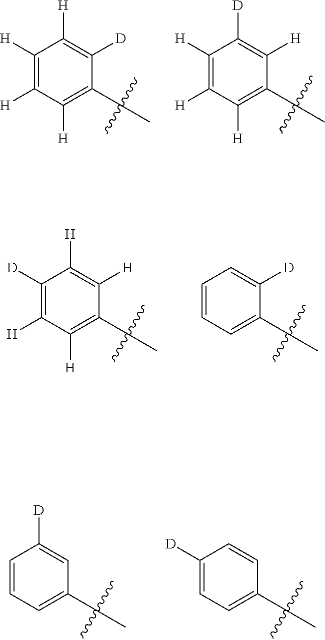 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

Further, in an exemplary embodiment of the present application, “a phenyl group having a deuterium content of 0%” may mean a phenyl group that does not include a deuterium atom, that is, has five hydrogen atoms.

In the present specification, the halogen may be fluorine, chlorine, bromine or iodine.

In the present specification, an alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, an alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like, but are not limited thereto.

In the present specification, an alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, a haloalkyl group means an alkyl group substituted with a halogen group, and specific examples thereof include —CF₃, —CF₂CF₃, and the like, but are not limited thereto.

In the present specification, an alkoxy group is represented by —O(R101), and the above-described examples of the alkyl group may be applied to R101.

In the present specification, an aryloxy group is represented by —O(R102), and the above-described examples of the aryl group may be applied to R102.

In the present specification, an alkylthioxy group is represented by —S(R103), and the above-described examples of the alkyl group may be applied to R103.

In the present specification, an arylthioxy group is represented by —S(R104), and the above-described examples of the aryl group may be applied to R104.

In the present specification, an alkylsulfoxy group is represented by —S(═O)₂(R105), and the above-described examples of the alkyl group may be applied to R105.

In the present specification, an arylsulfoxy group is represented by —S(═O)₂(R106), and the above-described examples of the aryl group may be applied to R106.

In the present specification, a cycloalkyl group includes a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, a heterocycloalkyl group includes O, S, Se, N, or Si as a heteroatom, includes a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, an aryl group includes a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, the terphenyl group may be selected from the following structures.

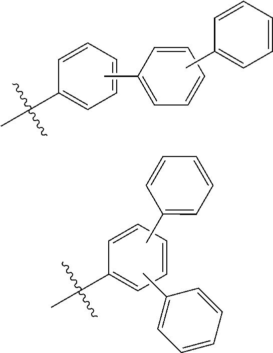

In the present specification, the fluorenyl group may be substituted, and adjacent substituents may be bonded to each other to form a ring.

When the fluorenyl group is substituted, the substituent may be

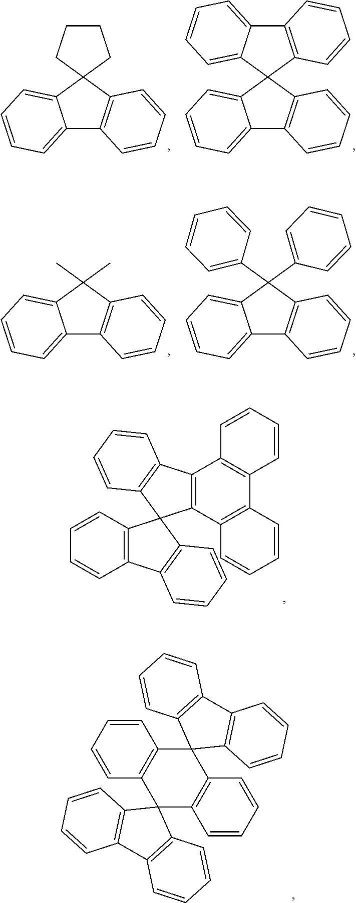

and the like, but is not limited thereto.

In the present specification, a heteroaryl group includes S, O, Se, N, or Si as a heteroatom, includes a monocycle or polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridine group, a pyrrole group, a pyrimidine group, a pyridazine group, a furan group, a thiophene group, an imidazole group, a pyrazole group, an oxazole group, an isoxazole group, a thiazole group, an isothiazole group, a triazole group, a furazan group, an oxadiazole group, a thiadiazole group, a dithiazole group, a tetrazolyl group, a pyran group, a thiopyran group, a diazine group, an oxazine group, a thiazine group, a dioxin group, a triazine group, a tetrazine group, a quinoline group, an isoquinoline group, a quinazoline group, an isoquinazoline group, a quinozoline group, a naphthyridine group, an acridine group, a phenanthridine group, an imidazopyridine group, a diazanaphthalene group, a triazaindene group, an indole group, an indolizine group, a benzothiazole group, a benzoxazole group, a benzimidazole group, a benzothiophene group, a benzofuran group, a dibenzothiophene group, a dibenzofuran group, a carbazole group, a benzocarbazole group, a dibenzocarbazole group, a phenazine group, a dibenzosilole group, spirobi(dibenzosilole), a dihydrophenazine group, a phenoxazine group, a phenanthridine group, a thienyl group, an indolo[2,3-a]carbazole group, an indolo[2,3-b]carbazole group, an indoline group, a 10,11-dihydro-dibenzo[b,f]azepine group, a 9,10-dihydroacridine group, a phenanthrazine group, a phenothiazine group, a phthalazine group, a phenanthroline group, a naphthobenzofuran group, a naphthobenzothiophene group, a benzo[c][1,2,5]thiadiazole group, a 2,3-dihydrobenzo[b]thiophene group, a 2,3-dihydrobenzofuran group, a 5,10-dihydrodibenzo[b,e][1,4]azasiline group, a pyrazolo[1,5-c]quinazoline group, a pyrido[1,2-b]indazole group, a pyrido[1,2-a]imidazo[1,2-e]indoline group, a 5,11-dihydroindeno[1,2-b]carbazole group, a 12H-benzofuro[2,3-a]carbazole group, a benzofuro[3,2-d]pyrimidine group, a benzo[4,5]thieno[3,2-d]pyrimidine group, and the like, but are not limited thereto.

In the present specification, when the substituent is a carbazole group, it means being bonded to nitrogen or carbon of carbazole.

In the present specification, when a carbazole group is substituted, an additional substituent may be substituted with the nitrogen or carbon of the carbazole.

In the present specification, a benzocarbazole group may be any one of the following structures.

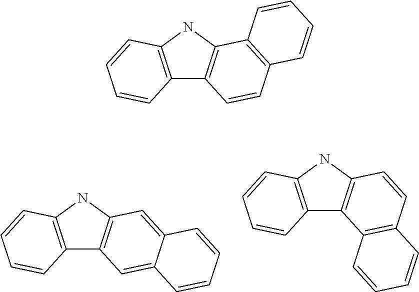

In the present specification, a dibenzocarbazole group may be any one of the following structures.

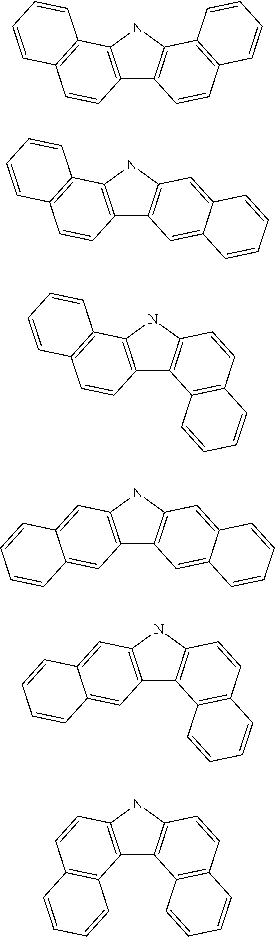 [SMILES: c1ccc2c(c1)ccc1c3ccc4ccccc4c3[nH]c21 ; c1ccc2cc3c(cc2c1)[nH]c1c2ccccc2ccc31 ; c1ccc2c(c1)ccc1c2[nH]c2ccc3ccccc3c21 ; c1ccc2cc3c(cc2c1)[nH]c1cc2ccccc2cc13 ; c1ccc2cc3c(cc2c1)[nH]c1ccc2ccccc2c13 ; c1ccc2c(c1)ccc1[nH]c3ccc4ccccc4c3c12]

In the present specification, a naphthobenzofuran group may be any one of the following structures.

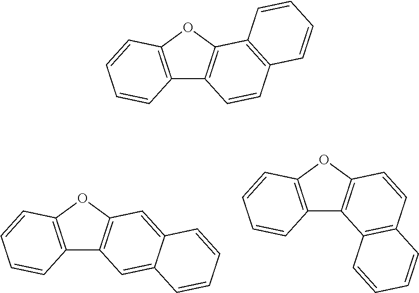

In the present specification, a naphthobenzothiophene group may be any one of the following structures.

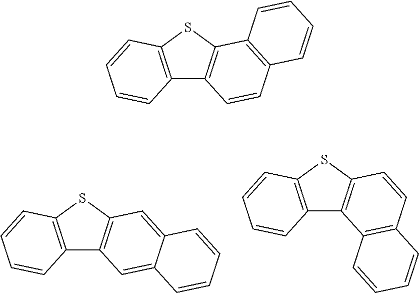

In the present specification, a silyl group includes Si and is a substituent to which the Si atom is directly linked as a radical, and is represented by —Si(R107) (R108) (R109), and R107 to R109 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specific examples of the silyl group include

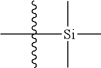 [SMILES: *[Si](C)(C)C]

(a trimethylsilyl group),

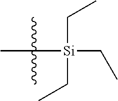 [SMILES: *[Si](CC)(CC)CC]

(a triethylsilyl group),

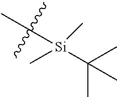 [SMILES: *[Si](C)(C)C(C)(C)C]

(a t-butyldimethylsilyl group),

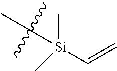 [SMILES: *[Si](C)(C)C=C]

(a vinyldimethylsilyl group),

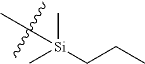 [SMILES: *[Si](C)(C)CCC]

(a propyldimethylsilyl group),

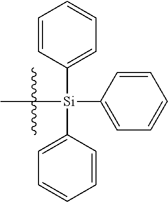 [SMILES: *[Si](c1ccccc1)(c1ccccc1)c1ccccc1]

(a triphenylsilyl group),

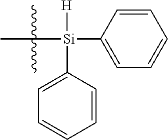 [SMILES: *[SiH](c1ccccc1)c1ccccc1]

(a diphenylsilyl group),

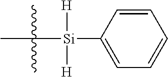 [SMILES: *[SiH2]c1ccccc1]

(a phenylsilyl group) and the like, but are not limited thereto.

In the present specification, a phosphine oxide group is represented by —P(═O) (R110) (R111), and R110 and R111 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and the above-described example may be applied to the alkyl group and the aryl group. Examples of the phosphine oxide group include a dimethylphosphine oxide group, a diphenylphosphine oxide group, dinaphthylphosphine oxide, and the like, but are not limited thereto.

In the present specification, an amine group is represented by —N(R112) (R113), and R112 and R113 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, the above-described examples of the aryl group may be applied to an arylene group except for a divalent arylene group.

In the present specification, the above-described examples of the heteroaryl group may be applied to a heteroarylene group except for a divalent heteroarylene group.

In the present specification, the “adjacent” group may mean a substituent substituted with an atom directly linked to an atom in which the corresponding substituent is substituted, a substituent disposed to be sterically closest to the corresponding substituent, or another substituent substituted with an atom in which the corresponding substituent is substituted. For example, two substituents substituted at the ortho position in a benzene ring and two substituents substituted at the same carbon in an aliphatic ring may be interpreted as groups which are “adjacent” to each other.

Hydrocarbon rings and hetero rings that adjacent groups may form include an aliphatic hydrocarbon ring, an aromatic hydrocarbon ring, an aliphatic hetero ring and an aromatic hetero ring, and structures exemplified by the above-described cycloalkyl group, aryl group, heterocycloalkyl group and heteroaryl group may be applied to the rings, except for those that are not monovalent groups.

In an exemplary embodiment of the present application, a group not represented by a substituent; or a group represented by hydrogen may mean being all substitutable with deuterium. That is, it may be shown that hydrogen; or deuterium can be substituted with each other.

In general, compounds bonded with hydrogen and compounds substituted with deuterium exhibit a difference in thermodynamic behavior. The reason for this is that the mass of a deuterium atom is 2-fold higher than that of hydrogen, but due to the difference in the mass of atoms, deuterium is characterized by having even lower vibration energy.

Further, the single bond dissociation energy of carbon and deuterium is higher than the single bond dissociation energy of carbon and hydrogen. Accordingly, the deuterium-substituted structure has an effect of increasing the thermal stability of the molecule and improving the service life of the device using the increased thermal stability.

When a compound is deposited on a silicon wafer, a material including deuterium tends to be packed so that the intermolecular distance is reduced. Further, when the surface of a thin film is observed using an atomic force microscope (AFM), it can be confirmed that the thin film made of a compound including deuterium is deposited with a more uniform surface without any aggregated portion.

The compounds of Chemical Formulae 1 and 2 of the present application has a deuterium substitution rate of 0% or 15% to 100% or less. The deuterium-substituted compound is characterized in that the energy in the ground state is lower than that of the hydrogen-substituted compound, and the shorter the bond length between carbon and deuterium is, the smaller the molecular hardcore volume is. Accordingly, the electrical polarizability may be reduced and the intermolecular interaction can be weakened, so that when a device is manufactured, the device has a stabler stacking structure.

These characteristics induce an effect of lowering the crystallinity by creating the amorphous state of a thin film. That is, compounds of Chemical Formulae 1 and 2 may be effective in improving the heat resistance of an OLED device, thereby improving the service life and driving characteristics.

The present application provides an organic light emitting device including: a first electrode; a second electrode; and an organic material layer provided between the first electrode and the second electrode, in which the organic material layer includes a compound of the following Chemical Formula 1 and a compound of the following Chemical Formula 2.

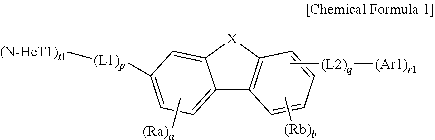

X is O or S,

Ar1 is a substituted or unsubstituted C6 to C60 aryl group; or a substituted or unsubstituted C2 to C60 heteroaryl group,

N-Het1 is a C2 to C60 heteroaryl group which is substituted or unsubstituted and includes N,

t1 and r1 are each an integer from 1 to 3, and when t1 is 2 or higher, N-Het1's are the same as or different from each other, and when r1 is 2 or higher, Ar1's are the same as or different from each other,

L1 and L2 are the same as or different from each other, and are each independently a direct bond; a substituted or unsubstituted C6 to C60 arylene group; or a substituted or unsubstituted C2 to C60 heteroarylene group,

p and q are each an integer from 0 to 3, and when p is 2 or higher, L1's are the same as or different from each other, and when q is 2 or higher, L2's are the same as or different from each other,

Ra and Rb are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and

a and b are each an integer from 0 to 3, and when a is 2 or higher, Ra's are the same as or different from each other, and when b is 2 or higher, Rb's are the same as or different from each other,

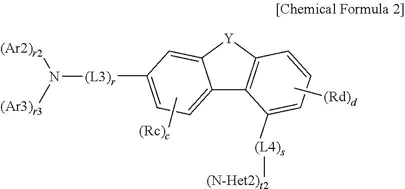

Y is O or S,

Ar2 and Ar3 are the same as or different from each other, and are each independently a substituted or unsubstituted C6 to C60 aryl group; or a substituted or unsubstituted C2 to C60 heteroaryl group,

N-Het2 is a C2 to C60 heteroaryl group which is substituted or unsubstituted and includes N,

t2, r2 and r3 are each an integer from 1 to 3, and when t2 is 2 or higher, N-Het2's are the same as or different from each other, and when r2 is 2 or higher, Ar2's are the same as or different from each other, and when r3 is 2 or higher, Ar3's are the same as or different from each other,

L3 and L4 are the same as or different from each other, and are each independently a direct bond; a substituted or unsubstituted C6 to C60 arylene group; or a substituted or unsubstituted C2 to C60 heteroarylene group,

r and s are each an integer from 0 to 3, and when r is 2 or higher, L3's are the same as or different from each other, and when s is 2 or higher, L4's are the same as or different from each other,

Rc and Rd are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and

c and d are each an integer from 0 to 3, and when c is 2 or higher, Rc's are the same as or different from each other, and when d is 2 or higher, Rd's are the same as or different from each other.

In an exemplary embodiment of the present application, Chemical Formula 1 may be represented by any one of the following Chemical Formulae 1-1 to 1-3.

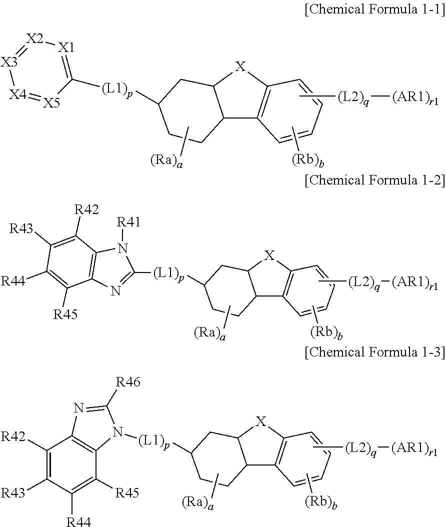

In Chemical Formulae 1-1 to 1-3, the definitions of X, L1, L2, Ar1, Ra, Rb, r1, p, q, a and b are the same as the definitions in Chemical Formula 1,

X1 is CR11 or N, X2 is CR12 or N, X3 is CR13 or N, X4 is CR14 or N, X5 is CR15 or N, and at least one of X1 to X5 is N, and

R11 to R15 and R41 to R46 are the same as or different from each other, and are each independently hydrogen; deuterium; a halogen; a cyano group; a substituted or unsubstituted C1 to C60 alkyl group; a substituted or unsubstituted C3 to C60 cycloalkyl group; a substituted or unsubstituted C3 to C60 heterocycloalkyl group; a substituted or unsubstituted C6 to C60 aryl group; or a substituted or unsubstituted C2 to C60 heteroaryl group.

In an exemplary embodiment of the present application, Chemical Formula 1 may be represented by any one of the following Chemical Formulae 1-4 to 1-6.

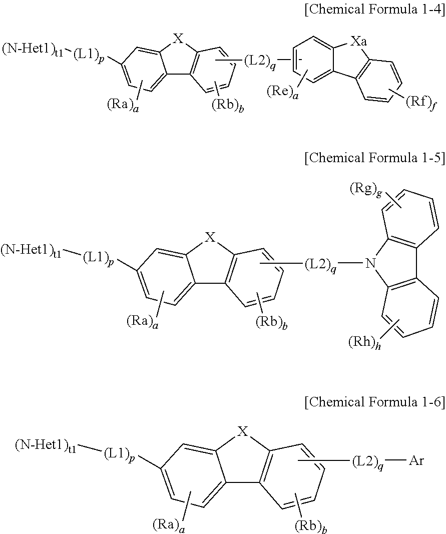

In Chemical Formulae 1-4 to 1-6,

the definitions of X, L1, L2, N-Het1, Ra, Rb, t1, p, q, a and b are the same as the definitions in Chemical Formula 1,

Xa is S, O or N(R51),

Re, Rf, Rg, Rh and R51 are the same as or different from each other, and are each independently selected from the group consisting of hydrogen; deuterium; a halogen; a cyano group; a substituted or unsubstituted C1 to C60 alkyl group; a substituted or unsubstituted C3 to C60 cycloalkyl group; a substituted or unsubstituted C3 to C60 heterocycloalkyl group; a substituted or unsubstituted C6 to C60 aryl group; a substituted or unsubstituted C2 to C60 heteroaryl group; a substituted or unsubstituted phosphine oxide group; and a substituted or unsubstituted amine group, or two or more adjacent groups are bonded to each other to form a substituted or unsubstituted aliphatic or aromatic hydrocarbon ring or hetero ring,

e is an integer from 0 to 3, f, g and h are each an integer from 0 to 4, and when e is 2 or higher, Re's are the same as or different from each other, and when f is 2 or higher, Rf's are the same as or different from each other, and when g is 2 or higher, Rg's are the same as or different from each other, and when h is 2 or higher, Rh's are the same as or different from each other, and

Ar is a substituted or unsubstituted C6 to C60 aryl group.

In an exemplary embodiment of the present application, Chemical Formula 1 may be represented by any one of the following Chemical Formulae 1-7 and 1-8.

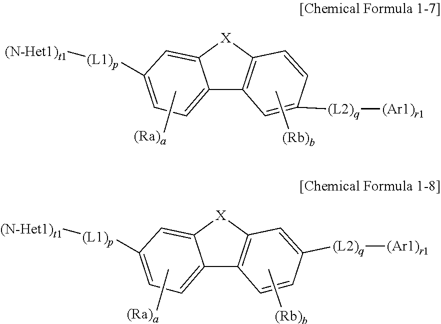

In Chemical Formulae 1-7 and 1-8,

the definitions of X, L1, L2, Ar1, N-Het1, Ra, Rb, t1, r1, p, q, a and b are the same as the definitions in Chemical Formula 1.

In an exemplary embodiment of the present application, Chemical Formula 1-1 may be represented by the following Chemical Formula 1-1-1.

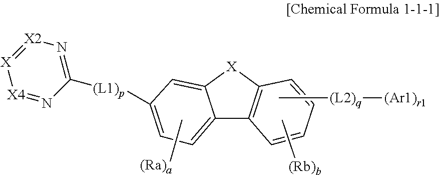

In Chemical Formula 1-1-1, the definitions of X, X2, X4, L1, L2, Ar1, Ra, Rb, p, q, a and b are the same as the definitions in Chemical Formula 1-1.

In an exemplary embodiment of the present application, when Rg or Rh in

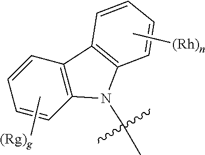

(here,

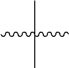

is a moiety linked to L2.) is bonded to an adjacent group to form a ring, the ring may be represented by the following Chemical Formula 1-5-1.

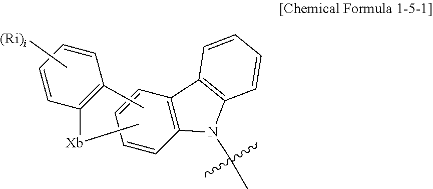

In Chemical Formula 1-5-1,

Xb is O, S, N(R17) or C(R18) (R19),

R17 to R19 are the same as or different from each other, and are each independently hydrogen; deuterium; a halogen; a cyano group; a substituted or unsubstituted C1 to C60 alkyl group; a substituted or unsubstituted C6 to C60 aryl group; or a substituted or unsubstituted C2 to C60 heteroaryl group.

In an exemplary embodiment of the present application, R17 to R19 are the same as or different from each other, and may be each independently hydrogen; deuterium; a substituted or unsubstituted methyl group; a substituted or unsubstituted ethyl group; a substituted or unsubstituted phenyl group; a substituted or unsubstituted biphenyl group; or a substituted or unsubstituted naphthyl group.

In yet another exemplary embodiment, Chemical Formula 1-5-1 may be selected from the following structural formulae.

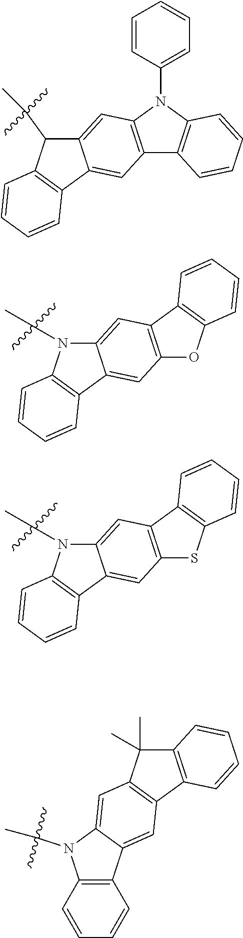 [SMILES: *C1c2ccccc2-c2cc3c4ccccc4n(-c4ccccc4)c3cc21 ; *n1c2ccccc2c2cc3oc4ccccc4c3cc21 ; *n1c2ccccc2c2cc3sc4ccccc4c3cc21 ; *n1c2ccccc2c2cc3c(cc21)C(C)(C)c1ccccc1-3]

In an exemplary embodiment of the present application, X may be O or S.

In an exemplary embodiment of the present application, the N-Het1 may be a C2 to C40 heteroaryl group which is substituted or unsubstituted and includes N.

In another exemplary embodiment, the N-Het1 may be a C2 to C20 heteroaryl group which is substituted or unsubstituted and includes N.

In still another exemplary embodiment, the N-Het1 may be a substituted or unsubstituted pyridine group; a substituted or unsubstituted pyrimidine group; a substituted or unsubstituted triazine group; or a substituted or unsubstituted benzimidazole group.

In yet another exemplary embodiment, the N-Het1 may be a triazine group which is substituted with an aryl group or a heteroaryl group.

In still yet another exemplary embodiment, the N-Het1 may be deuterium, a naphthyl group, a phenyl group unsubstituted or substituted with a dibenzofuran group, a biphenyl group unsubstituted or substituted with deuterium, a terphenyl group unsubstituted or substituted with deuterium, a naphthyl group unsubstituted or substituted with a phenyl group, a dibenzofuran group unsubstituted or substituted with deuterium, a dibenzothiophene group or a triazine group substituted with a carbazole group substituted with a phenyl group.

In a further exemplary embodiment, L1 and L2 are the same as or different from each other, and may be each independently a direct bond; a substituted or unsubstituted C6 to C40 arylene group; or a substituted or unsubstituted C2 to C40 heteroarylene group.

In another further exemplary embodiment, L1 and L2 are the same as or different from each other, and may be each independently a direct bond; or a substituted or unsubstituted C6 to C20 arylene group.

In still another further exemplary embodiment, L1 and L2 are the same as or different from each other, and may be each independently a direct bond; a substituted or unsubstituted phenylene group; a substituted or unsubstituted biphenylene group; or a substituted or unsubstituted naphthylene group.

In yet another further exemplary embodiment, L1 and L2 are the same as or different from each other, and may be each independently a direct bond; a phenylene group; a biphenylene group; or a naphthylene group.

In an exemplary embodiment of the present application, p and q are each an integer from 0 to 2, and when p is 2 or higher, L1's are the same as or different from each other, and when q is 2 or higher, L2's may be the same as or different from each other.

In an exemplary embodiment of the present application, Ra and Rb are the same as or different from each other, and may be each independently hydrogen; deuterium; a halogen group; a cyano group; a substituted or unsubstituted C1 to C20 alkyl group; a substituted or unsubstituted C6 to C20 aryl group; or a substituted or unsubstituted C2 to C20 heteroaryl group.

In another exemplary embodiment, Ra and Rb are the same as or different from each other, and may be each independently hydrogen; deuterium; a substituted or unsubstituted phenyl group or a substituted or unsubstituted biphenyl group.

In still another exemplary embodiment, Ra and Rb are the same as or different from each other, and may be each independently hydrogen; or deuterium.

In yet another exemplary embodiment, a and b are each an integer from 0 to 2, and when a is 2 or higher, Ra's are the same as or different from each other, and when b is 2 or higher, Rb's may be the same as or different from each other.

In an exemplary embodiment of the present application, Ar1 may be a substituted or unsubstituted C6 to C40 aryl group; or a substituted or unsubstituted C2 to C40 heteroaryl group.

In another exemplary embodiment, Ar1 may be a substituted or unsubstituted C6 to C20 aryl group; or a substituted or unsubstituted C2 to C20 heteroaryl group.

In still another exemplary embodiment, Ar1 may be a substituted or unsubstituted phenyl group; substituted or unsubstituted biphenyl group; substituted or unsubstituted terphenyl group; a substituted or unsubstituted naphthyl group; a substituted or unsubstituted phenanthrene group; a substituted or unsubstituted triphenylene group; a substituted or unsubstituted fluorenyl group; a substituted or unsubstituted dibenzofuran group; a substituted or unsubstituted dibenzothiophene group; a substituted or unsubstituted carbazole group; a substituted or unsubstituted indolocarbazole group; a substituted or unsubstituted indenocarbazole group; a substituted or unsubstituted benzofurocarbazole group; or a substituted or unsubstituted benzothienocarbazole group.

In yet another exemplary embodiment, Ar1 may be a phenyl group which is unsubstituted or substituted with deuterium, a naphthyl group, a dibenzofuran group, a dibenzothiophene group or a carbazole group substituted with a phenyl group; a biphenyl group which is unsubstituted or substituted with deuterium; a terphenyl group; a naphthyl group which is unsubstituted or substituted with deuterium or a phenyl group; a phenanthrene group; a triphenylene group; a fluorenyl group which is unsubstituted or substituted with a methyl group or a phenyl group; a spirobifluorenyl group; a dibenzofuran group; a dibenzothiophene group; a carbazole group which is unsubstituted or substituted with a phenyl group; an indolocarbazole group which is unsubstituted or substituted with a phenyl group; an indenocarbazole group which is unsubstituted or substituted with a methyl group; a benzofurocarbazole group; or a benzothienocarbazole group.

In an exemplary embodiment of the present application, t1 and r1 are each an integer from 1 to 2, and when t1 is 2, N-Het1's are the same as or different from each other, and when r1 is 2, Ar1 's may be the same as or different from each other.

In another exemplary embodiment, t1 and r1 are 1.

In an exemplary embodiment of the present application, X1, X3 and X5 are N, X2 is CR12, and X4 may be CR14.

In an exemplary embodiment of the present application, R11 to R15 and R41 to R46 are the same as or different from each other, and may be each independently hydrogen; deuterium; a substituted or unsubstituted C6 to C40 aryl group; or a substituted or unsubstituted C2 to C40 heteroaryl group.

In another exemplary embodiment, R11 to R15 and R41 to R46 are the same as or different from each other, and may be each independently hydrogen; deuterium; a substituted or unsubstituted C6 to C20 aryl group; or a substituted or unsubstituted C2 to C20 heteroaryl group.

In still another exemplary embodiment, R11 to R15 are the same as or different from each other, and may be each independently a substituted or unsubstituted C6 to C20 aryl group; or a substituted or unsubstituted C2 to C20 heteroaryl group.

In yet another exemplary embodiment, R11 to R15 are the same as or different from each other, and may be each independently deuterium, a naphthyl group, a phenyl group unsubstituted or substituted with a dibenzofuran group, a biphenyl group unsubstituted or substituted with deuterium, a terphenyl group unsubstituted or substituted with deuterium, a naphthyl group unsubstituted or substituted with a phenyl group, a dibenzofuran group unsubstituted or substituted with deuterium, a dibenzothiophene group or a triazine group substituted with a carbazole group substituted with a phenyl group.

In still yet another exemplary embodiment, R41 to R46 are the same as or different from each other, and may be each independently hydrogen; or deuterium.

In an exemplary embodiment of the present application, Re, Rf, Rg, Rh and R51 are the same as or different from each other, and are each independently selected from the group consisting of hydrogen; deuterium; a substituted or unsubstituted C1 to C40 alkyl group; a substituted or unsubstituted C6 to C40 aryl group and a substituted or unsubstituted C2 to C40 heteroaryl group, or two or more adjacent groups may be bonded to each other to form a substituted or unsubstituted aliphatic or aromatic hydrocarbon ring or hetero ring.

In still another exemplary embodiment, Re, Rf, Rg, Rh and R51 are the same as or different from each other, and are each independently selected from the group consisting of hydrogen; deuterium; a substituted or unsubstituted C1 to C20 alkyl group and a substituted or unsubstituted C6 to C20 aryl group, or two or more adjacent groups may be bonded to each other to form a substituted or unsubstituted aliphatic or aromatic hydrocarbon ring or hetero ring.

In yet another exemplary embodiment, Re, Rf, Rg, Rh and R51 are the same as or different from each other, and are each independently selected from the group consisting of hydrogen; deuterium; a substituted or unsubstituted methyl group; a substituted or unsubstituted ethyl group; a substituted or unsubstituted propylene group; a substituted or unsubstituted phenyl group; a substituted or unsubstituted biphenyl group and a substituted or unsubstituted naphthyl group, or two or more adjacent groups may be bonded to each other to form a substituted or unsubstituted aliphatic or aromatic hydrocarbon ring or hetero ring.

In an exemplary embodiment of the present application, Ar2 may be a substituted or unsubstituted C6 to C40 aryl group.

In another exemplary embodiment, Ar may be a substituted or unsubstituted C6 to C20 aryl group.

In still another exemplary embodiment, Ar may be a substituted or unsubstituted phenyl group; a substituted or unsubstituted biphenyl group; a substituted or unsubstituted terphenyl group; a substituted or unsubstituted naphthyl group; a substituted or unsubstituted phenanthrene group; a substituted or unsubstituted triphenylene group; or a substituted or unsubstituted fluorenyl group.

In yet another exemplary embodiment, Ar may be a phenyl group which is unsubstituted or substituted with deuterium, a naphthyl group, a dibenzofuran group, a dibenzothiophene group or a carbazole group substituted with a phenyl group; a biphenyl group which is unsubstituted or substituted with deuterium; a terphenyl group; a naphthyl group which is unsubstituted or substituted with deuterium or a phenyl group; a phenanthrene group; a triphenylene group; a fluorenyl group which is unsubstituted or substituted with a methyl group or a phenyl group; or a spirobifluorenyl group.

In an exemplary embodiment of the present application, the deuterium content of the compound of Chemical Formula 1 may be 0%, or 1% to 100%.

In an exemplary embodiment of the present application, the deuterium content of the compound of Chemical Formula 1 may be 0%, or 10% to 100%.

In an exemplary embodiment of the present application, the deuterium content of the compound of Chemical Formula 1 may be 0%, or 15% to 100%.

In an exemplary embodiment of the present application, the deuterium content of the compound of Chemical Formula 1 may be 0%, or 30% to 100%.

In an exemplary embodiment of the present application, the deuterium content of the compound of Chemical Formula 1 may be 0%, or 50% to 100%.

In an exemplary embodiment of the present application, the deuterium content of the compound of Chemical Formula 1 may be 0%, or 70% to 100%.

In an exemplary embodiment of the present application, the deuterium content of the compound of Chemical Formula 1 may be 0%, or 90% to 100%.

In an exemplary embodiment of the present application, the deuterium content of the compound of Chemical Formula 1 may be 0%.

In an exemplary embodiment of the present application, the deuterium content of the compound of Chemical Formula 1 may be 15% to 100%.

In an exemplary embodiment of the present application, the deuterium content of the compound of Chemical Formula 1 may be 30% to 100%.

In an exemplary embodiment of the present application, the deuterium content of the compound of Chemical Formula 1 may be 50% to 100%.

In an exemplary embodiment of the present application, the deuterium content of the compound of Chemical Formula 1 may be 70% to 100%.

In an exemplary embodiment of the present application, the deuterium content of the compound of Chemical Formula 1 may be 90% to 100%.

In an exemplary embodiment of the present application, provided is a compound in which Chemical Formula 1 is represented by any one of the following compounds.

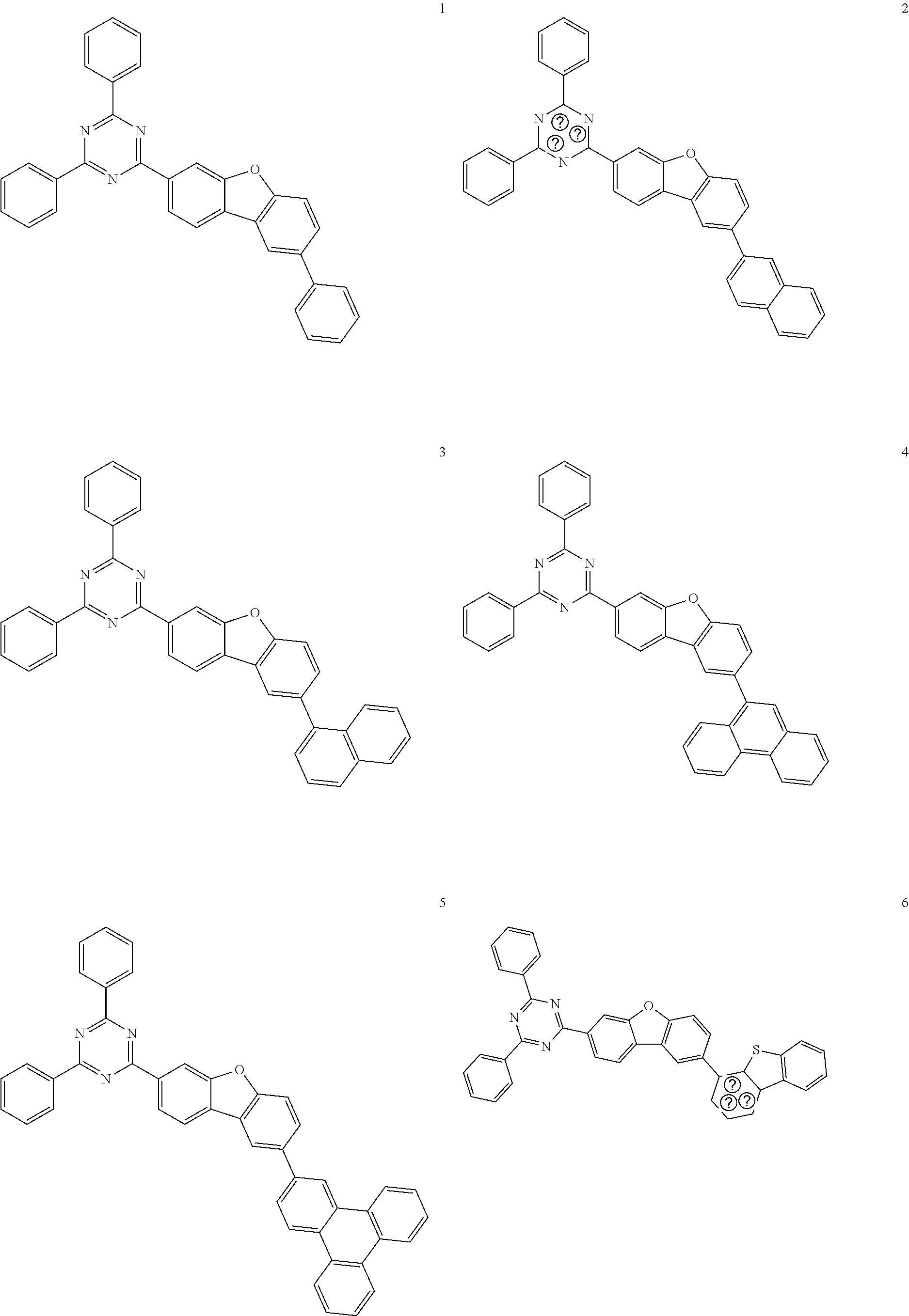 [SMILES: c1ccc(-c2ccc3oc4cc(-c5nc(-c6ccccc6)nc(-c6ccccc6)n5)ccc4c3c2)cc1 ; c1ccc(C2NC(c3ccccc3)NC(c3ccc4c(c3)oc3ccc(-c5ccc6ccccc6c5)cc34)N2)cc1 ; c1ccc(-c2nc(-c3ccccc3)nc(-c3ccc4c(c3)oc3ccc(-c5cccc6ccccc56)cc34)n2)cc1 ; c1ccc(-c2nc(-c3ccccc3)nc(-c3ccc4c(c3)oc3ccc(-c5cc6ccccc6c6ccccc56)cc34)n2)cc1 ; c1ccc(-c2nc(-c3ccccc3)nc(-c3ccc4c(c3)oc3ccc(-c5ccc6c7ccccc7c7ccccc7c6c5)cc34)n2)cc1 ; c1ccc(-c2nc(-c3ccccc3)nc(-c3ccc4c(c3)oc3ccc(C5CCCC6c7ccccc7SC56)cc34)n2)cc1]

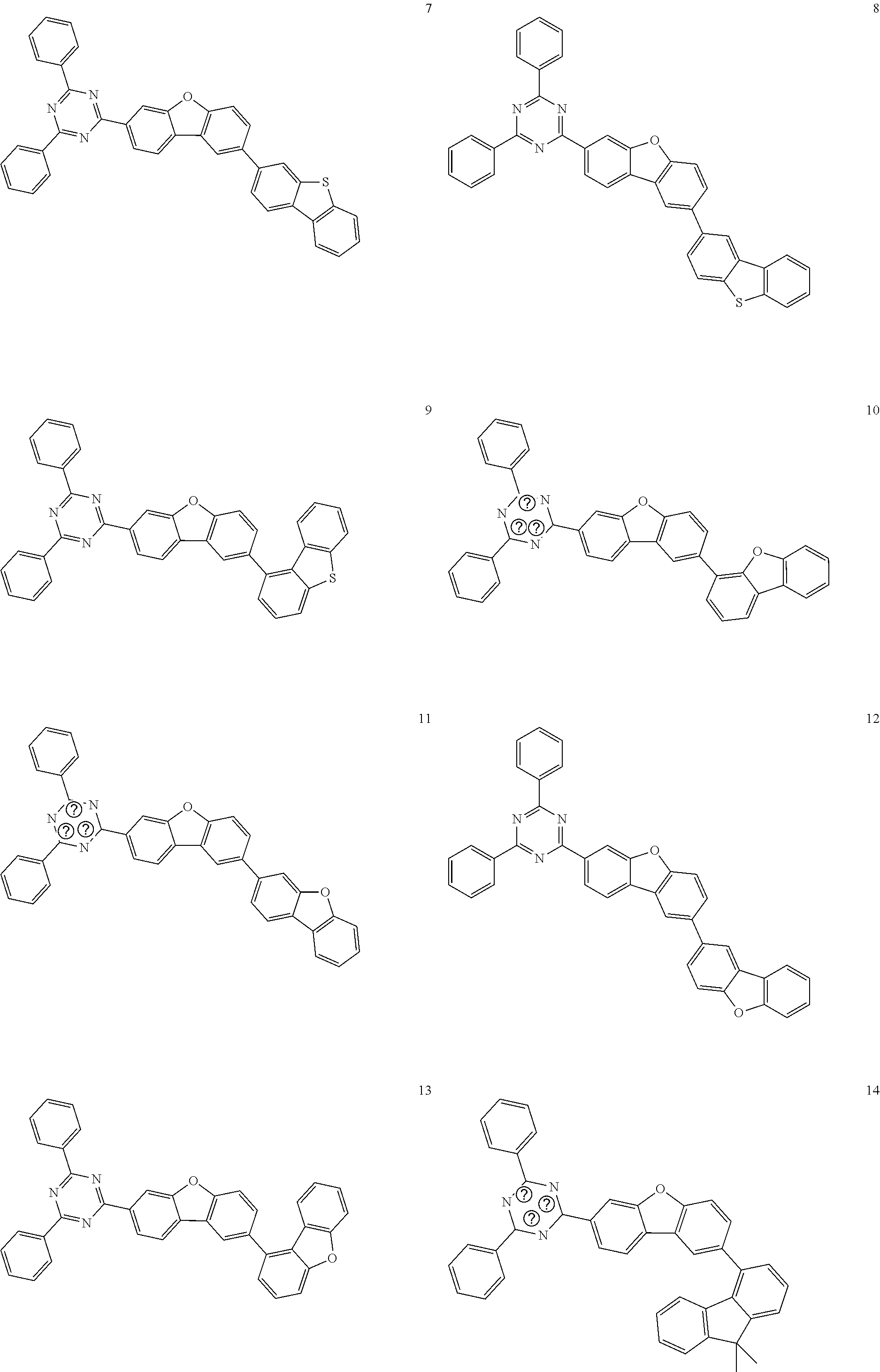

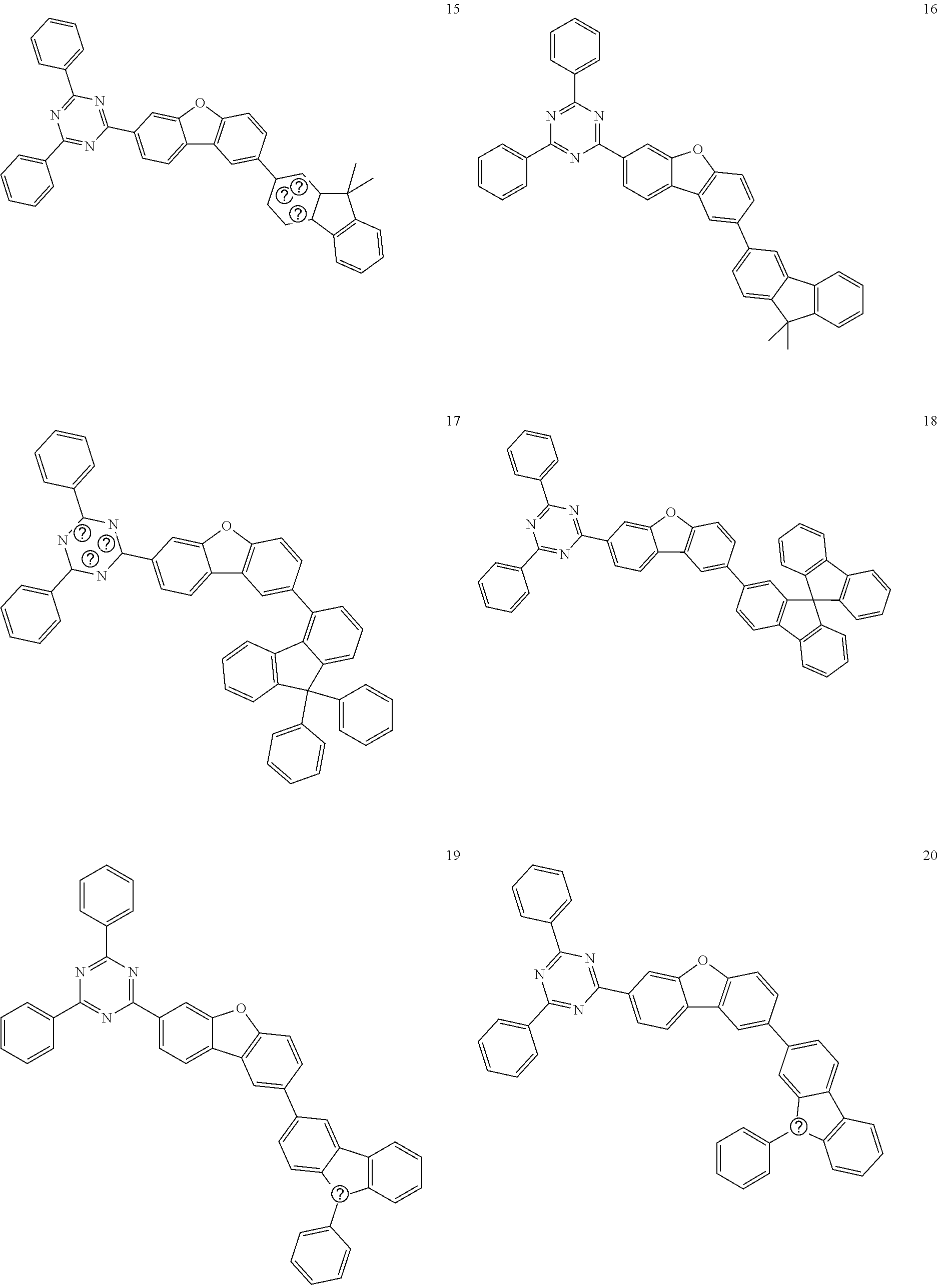

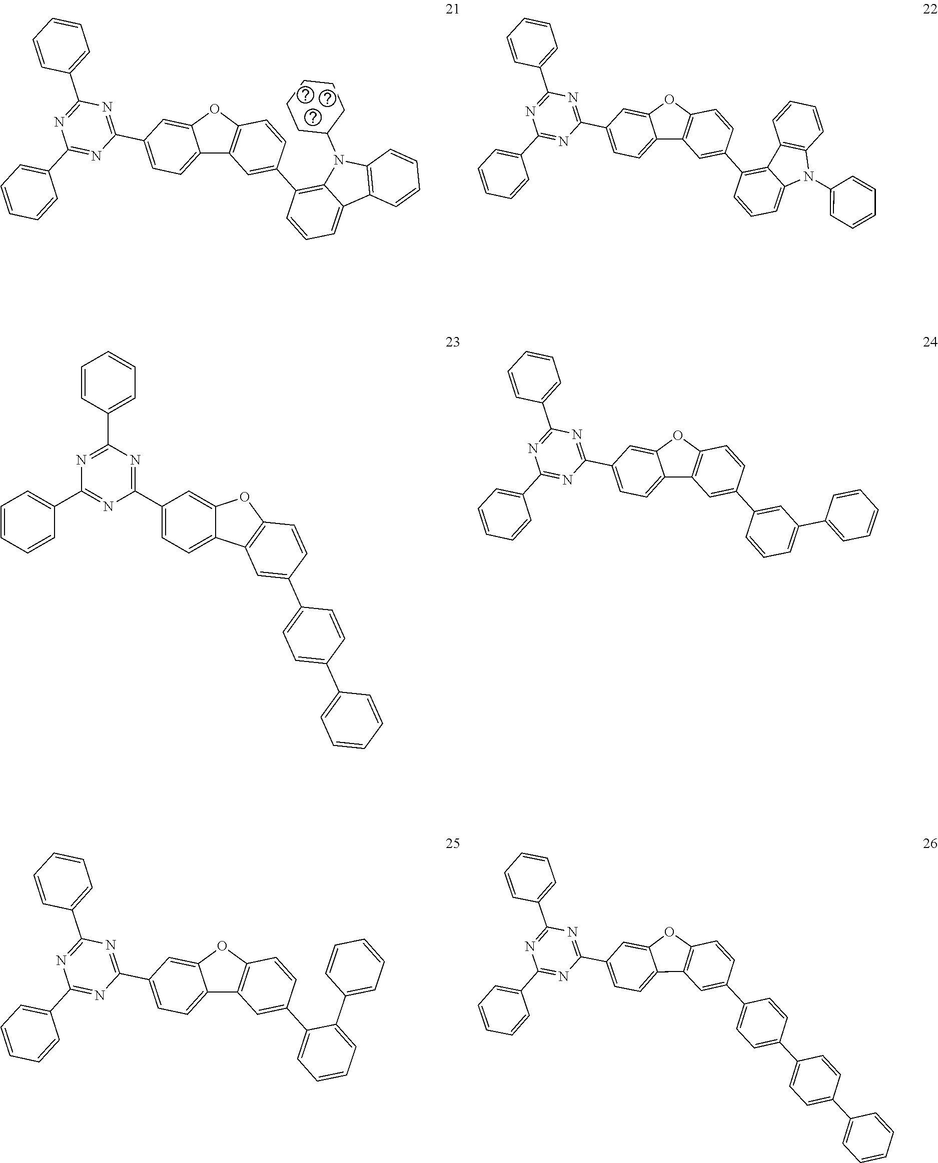 [SMILES: c1ccc(-c2nc(-c3ccccc3)nc(-c3ccc4c(c3)oc3ccc(-c5cccc6c7ccccc7n(C7CCCCC7)c56)cc34)n2)cc1 ; c1ccc(-c2nc(-c3ccccc3)nc(-c3ccc4c(c3)oc3ccc(-c5cccc6c5c5ccccc5n6-c5ccccc5)cc34)n2)cc1 ; c1ccc(-c2ccc(-c3ccc4oc5cc(-c6nc(-c7ccccc7)nc(-c7ccccc7)n6)ccc5c4c3)cc2)cc1 ; c1ccc(-c2cccc(-c3ccc4oc5cc(-c6nc(-c7ccccc7)nc(-c7ccccc7)n6)ccc5c4c3)c2)cc1 ; c1ccc(-c2nc(-c3ccccc3)nc(-c3ccc4c(c3)oc3ccc(-c5ccccc5-c5ccccc5)cc34)n2)cc1 ; c1ccc(-c2ccc(-c3ccc(-c4ccc5oc6cc(-c7nc(-c8ccccc8)nc(-c8ccccc8)n7)ccc6c5c4)cc3)cc2)cc1]

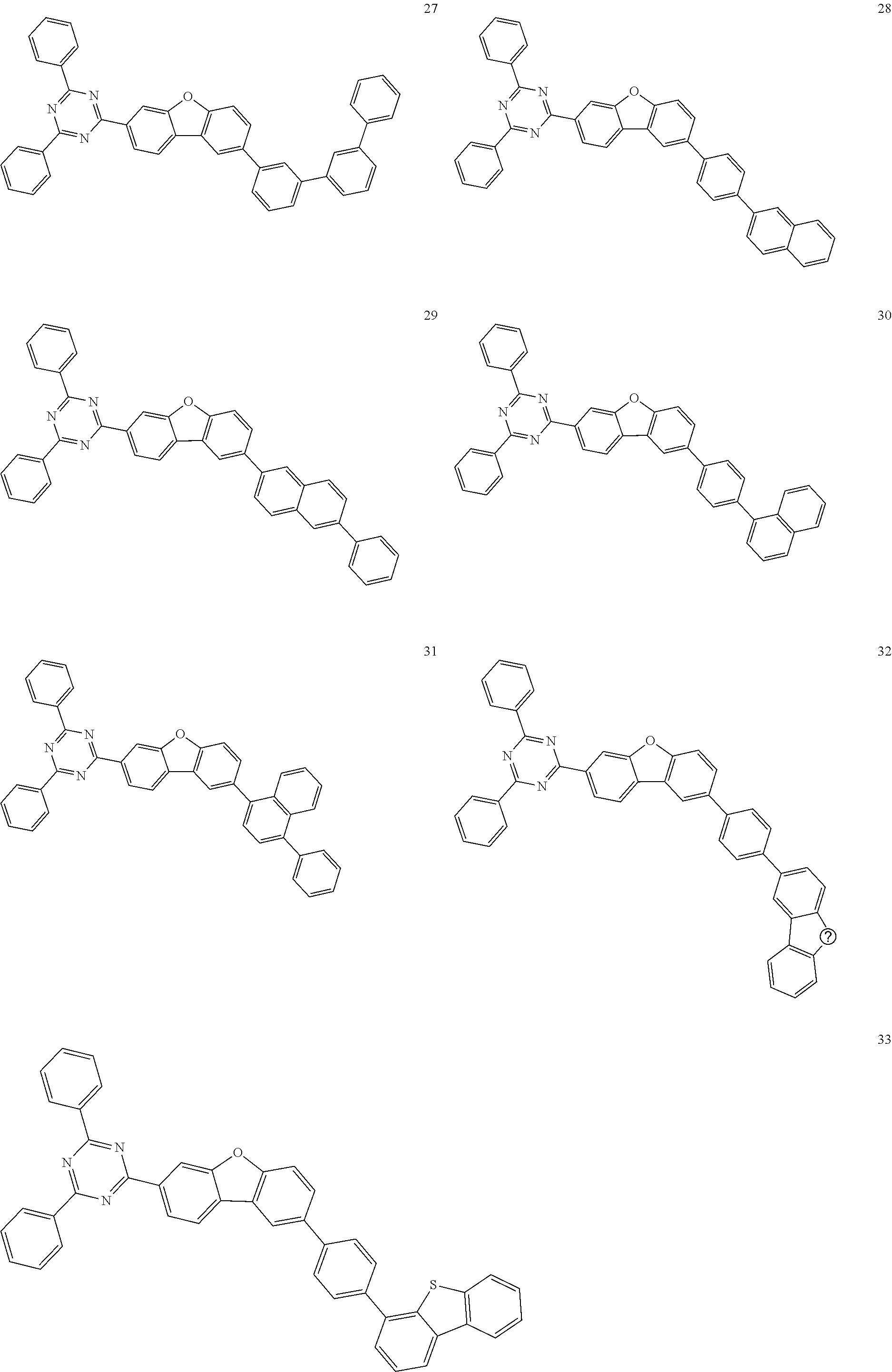

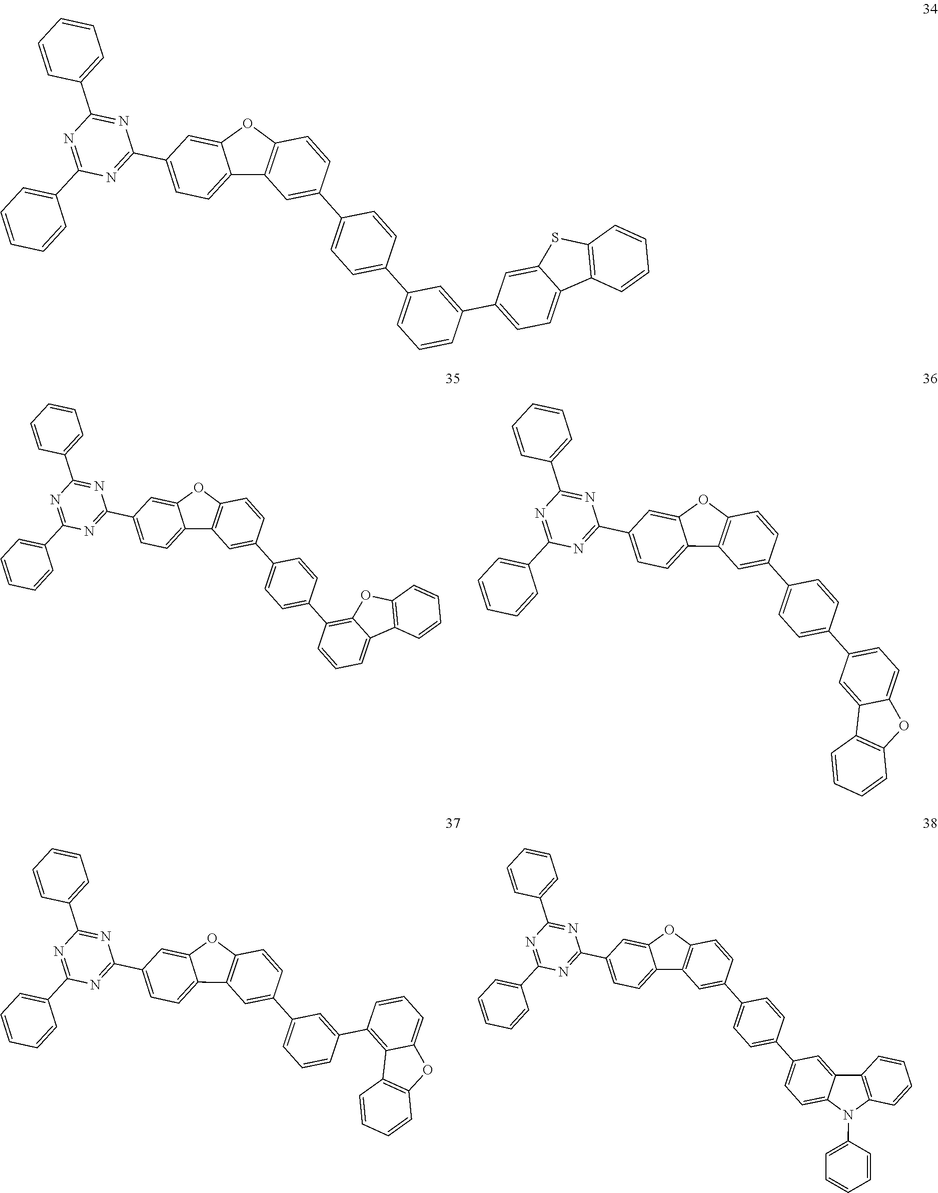 [SMILES: c1ccc(-c2nc(-c3ccccc3)nc(-c3ccc4c(c3)oc3ccc(-c5ccc(-c6cccc(-c7ccc8c(c7)sc7ccccc78)c6)cc5)cc34)n2)cc1 ; c1ccc(-c2nc(-c3ccccc3)nc(-c3ccc4c(c3)oc3ccc(-c5ccc(-c6cccc7c6oc6ccccc67)cc5)cc34)n2)cc1 ; c1ccc(-c2nc(-c3ccccc3)nc(-c3ccc4c(c3)oc3ccc(-c5ccc(-c6ccc7oc8ccccc8c7c6)cc5)cc34)n2)cc1 ; c1ccc(-c2nc(-c3ccccc3)nc(-c3ccc4c(c3)oc3ccc(-c5cccc(-c6cccc7oc8ccccc8c67)c5)cc34)n2)cc1 ; c1ccc(-c2nc(-c3ccccc3)nc(-c3ccc4c(c3)oc3ccc(-c5ccc(-c6ccc7c(c6)c6ccccc6n7-c6ccccc6)cc5)cc34)n2)cc1]

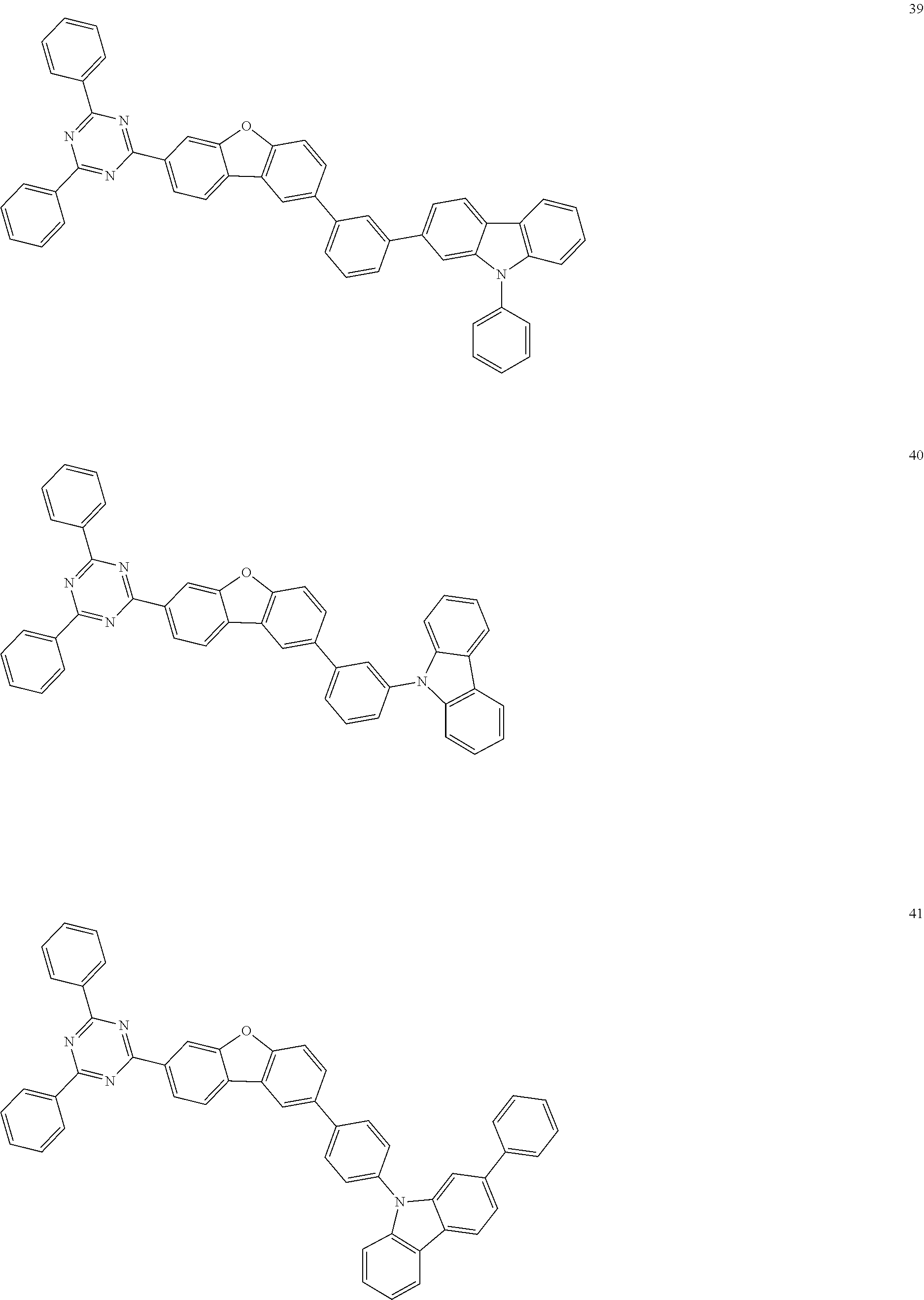 [SMILES: c1ccc(-c2nc(-c3ccccc3)nc(-c3ccc4c(c3)oc3ccc(-c5cccc(-c6ccc7c8ccccc8n(-c8ccccc8)c7c6)c5)cc34)n2)cc1 ; c1ccc(-c2nc(-c3ccccc3)nc(-c3ccc4c(c3)oc3ccc(-c5cccc(-n6c7ccccc7c7ccccc76)c5)cc34)n2)cc1 ; c1ccc(-c2ccc3c4ccccc4n(-c4ccc(-c5ccc6oc7cc(-c8nc(-c9ccccc9)nc(-c9ccccc9)n8)ccc7c6c5)cc4)c3c2)cc1]

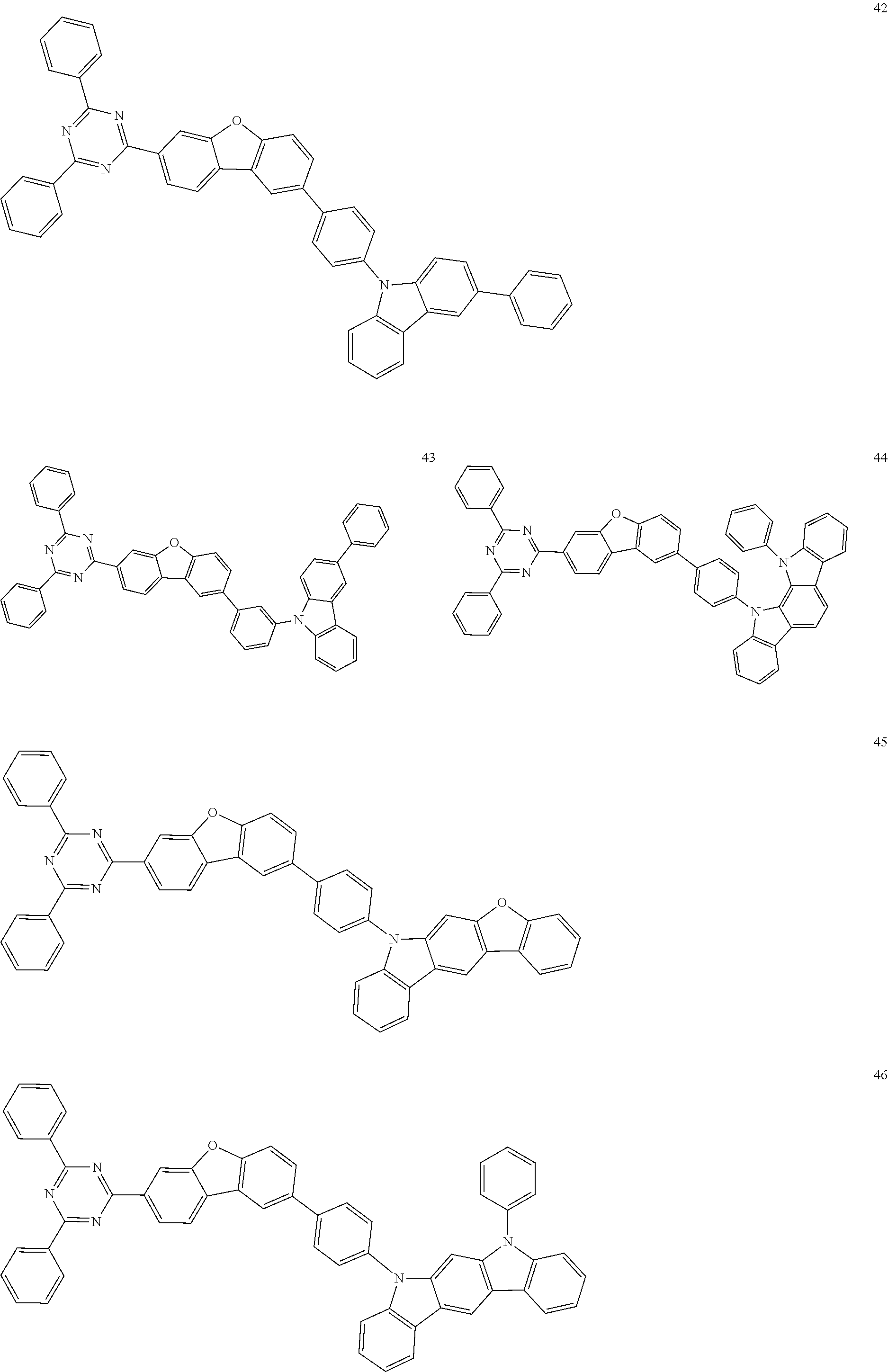

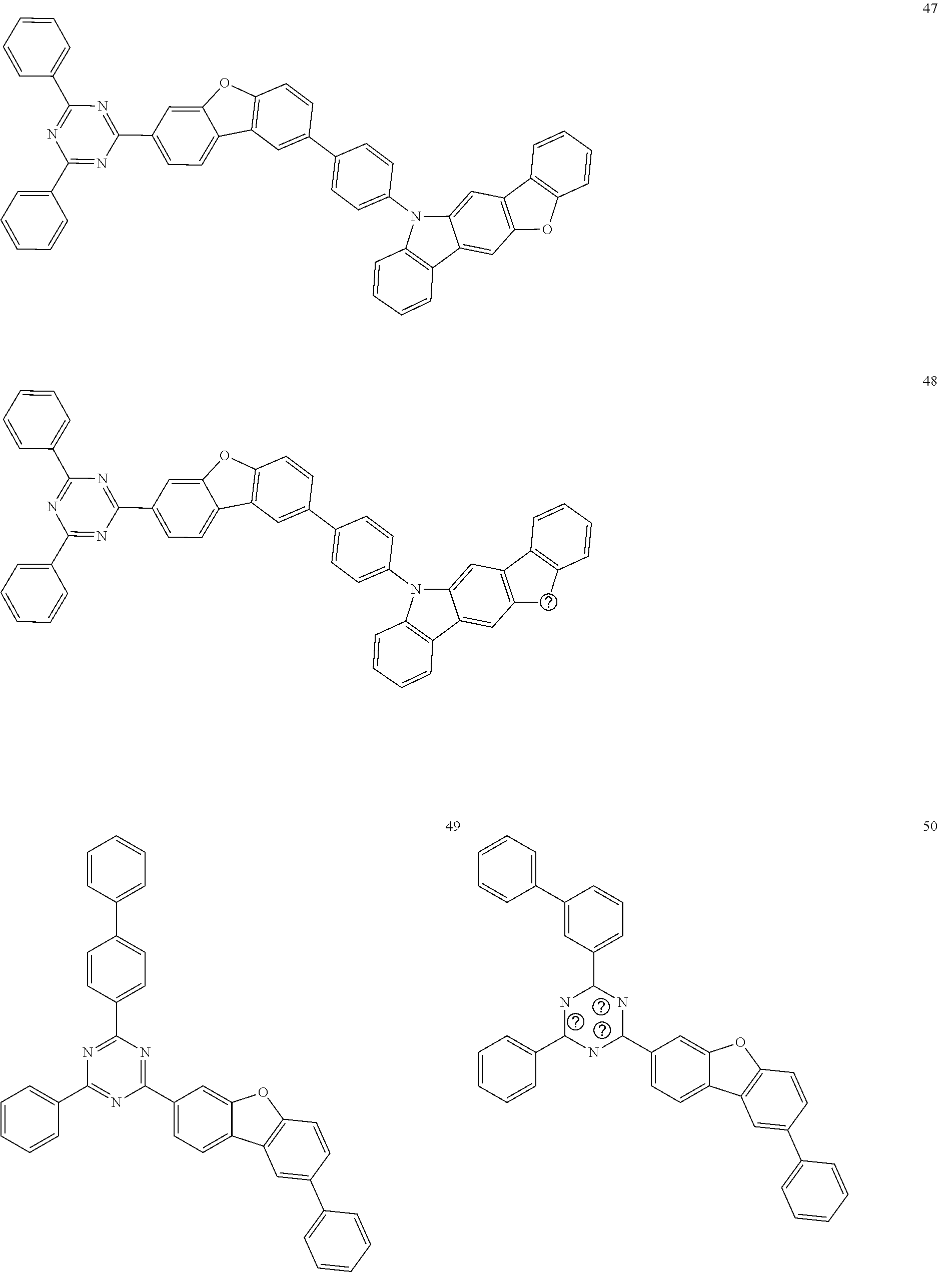 [SMILES: c1ccc(-c2nc(-c3ccccc3)nc(-c3ccc4c(c3)oc3ccc(-c5ccc(-n6c7ccccc7c7cc8oc9ccccc9c8cc76)cc5)cc34)n2)cc1 ; c1ccc(-c2nc(-c3ccccc3)nc(-c3ccc4c(c3)oc3ccc(-c5ccc(-n6c7ccccc7c7cc8c(cc76)-c6ccccc6C8)cc5)cc34)n2)cc1 ; c1ccc(-c2ccc(-c3nc(-c4ccccc4)nc(-c4ccc5c(c4)oc4ccc(-c6ccccc6)cc45)n3)cc2)cc1 ; c1ccc(-c2cccc(C3NC(c4ccccc4)NC(c4ccc5c(c4)oc4ccc(-c6ccccc6)cc45)N3)c2)cc1]

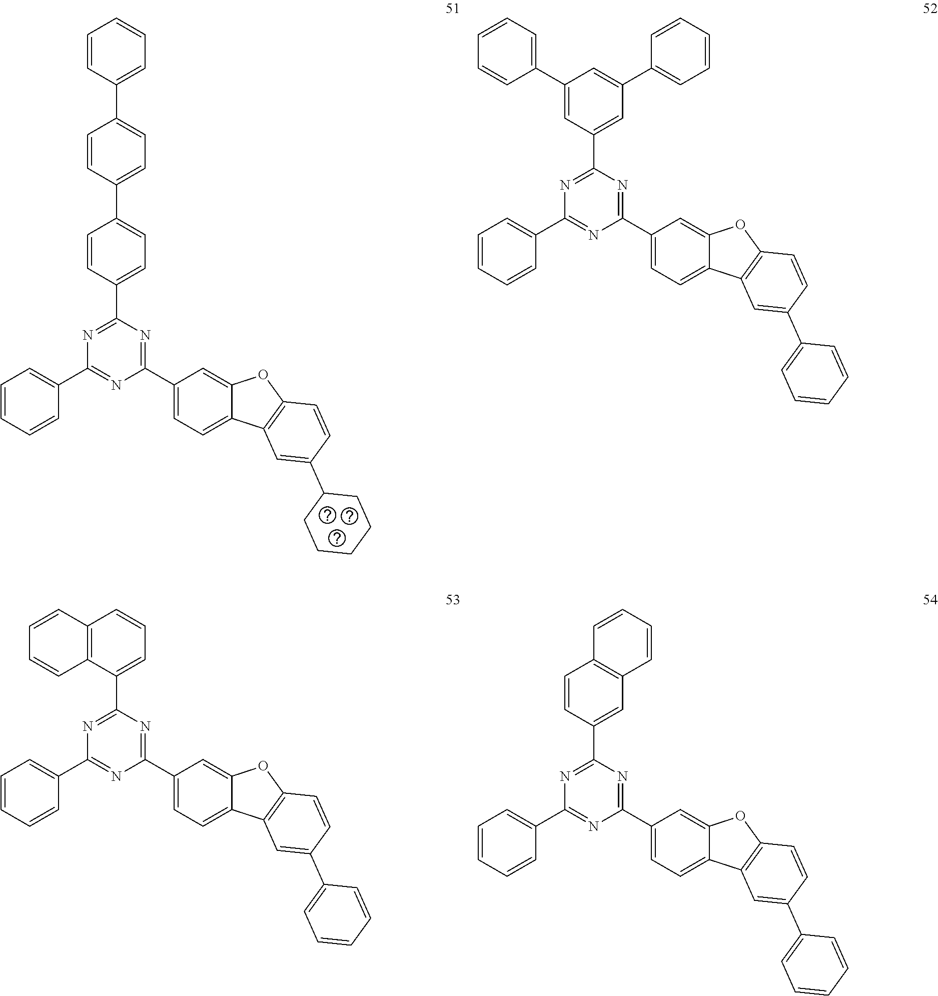 [SMILES: c1ccc(-c2ccc(-c3ccc(-c4nc(-c5ccccc5)nc(-c5ccc6c(c5)oc5ccc(C7CCCCC7)cc56)n4)cc3)cc2)cc1 ; c1ccc(-c2cc(-c3ccccc3)cc(-c3nc(-c4ccccc4)nc(-c4ccc5c(c4)oc4ccc(-c6ccccc6)cc45)n3)c2)cc1 ; c1ccc(-c2ccc3oc4cc(-c5nc(-c6ccccc6)nc(-c6cccc7ccccc67)n5)ccc4c3c2)cc1 ; c1ccc(-c2ccc3oc4cc(-c5nc(-c6ccccc6)nc(-c6ccc7ccccc7c6)n5)ccc4c3c2)cc1]

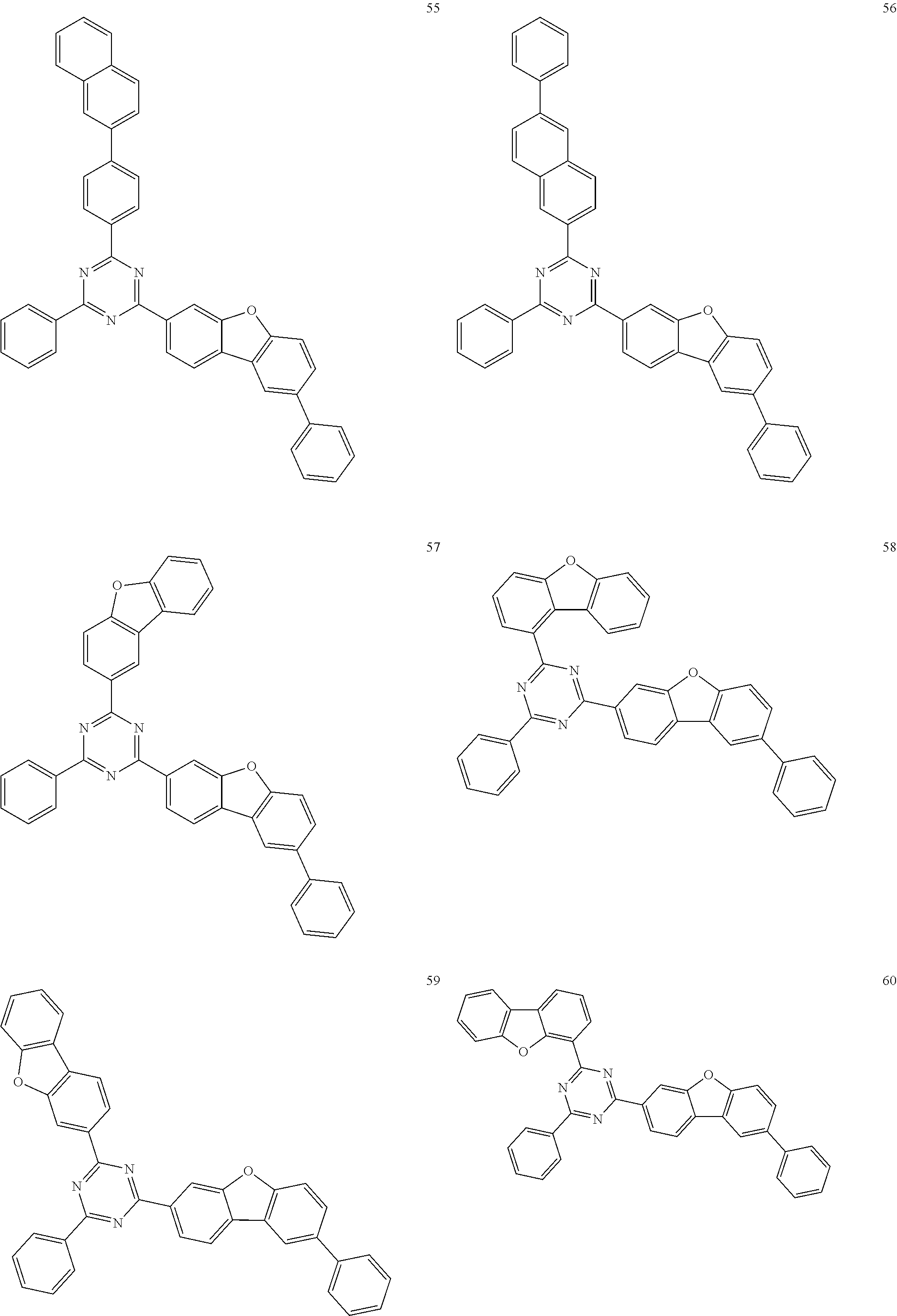 [SMILES: c1ccc(-c2ccc3oc4cc(-c5nc(-c6ccccc6)nc(-c6ccc(-c7ccc8ccccc8c7)cc6)n5)ccc4c3c2)cc1 ; c1ccc(-c2ccc3cc(-c4nc(-c5ccccc5)nc(-c5ccc6c(c5)oc5ccc(-c7ccccc7)cc56)n4)ccc3c2)cc1 ; c1ccc(-c2ccc3oc4cc(-c5nc(-c6ccccc6)nc(-c6ccc7oc8ccccc8c7c6)n5)ccc4c3c2)cc1 ; c1ccc(-c2ccc3oc4cc(-c5nc(-c6ccccc6)nc(-c6cccc7oc8ccccc8c67)n5)ccc4c3c2)cc1 ; c1ccc(-c2ccc3oc4cc(-c5nc(-c6ccccc6)nc(-c6ccc7c(c6)oc6ccccc67)n5)ccc4c3c2)cc1 ; c1ccc(-c2ccc3oc4cc(-c5nc(-c6ccccc6)nc(-c6cccc7c6oc6ccccc67)n5)ccc4c3c2)cc1]

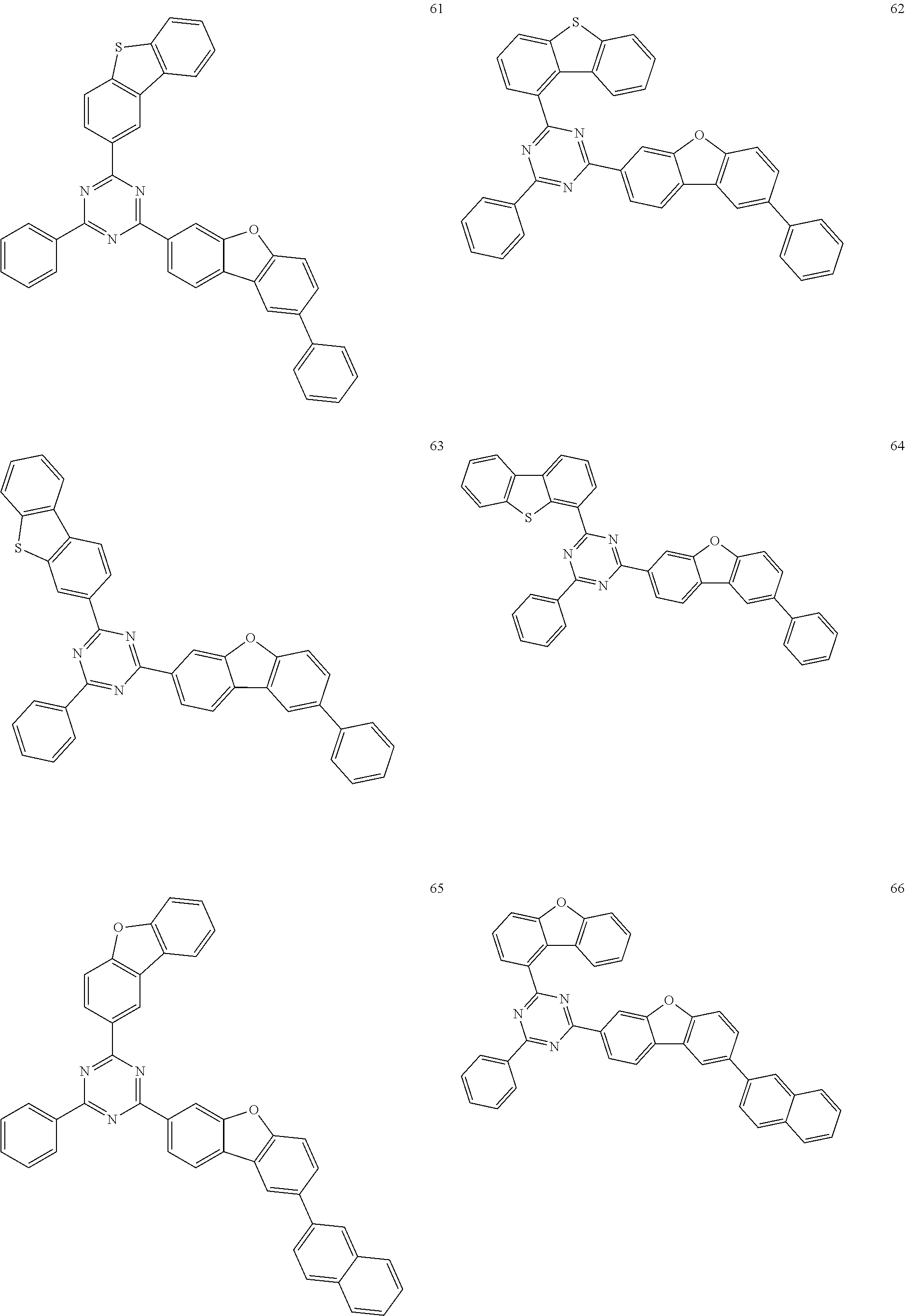 [SMILES: c1ccc(-c2ccc3oc4cc(-c5nc(-c6ccccc6)nc(-c6ccc7sc8ccccc8c7c6)n5)ccc4c3c2)cc1 ; c1ccc(-c2ccc3oc4cc(-c5nc(-c6ccccc6)nc(-c6cccc7sc8ccccc8c67)n5)ccc4c3c2)cc1 ; c1ccc(-c2ccc3oc4cc(-c5nc(-c6ccccc6)nc(-c6ccc7c(c6)sc6ccccc67)n5)ccc4c3c2)cc1 ; c1ccc(-c2ccc3oc4cc(-c5nc(-c6ccccc6)nc(-c6cccc7c6sc6ccccc67)n5)ccc4c3c2)cc1 ; c1ccc(-c2nc(-c3ccc4c(c3)oc3ccc(-c5ccc6ccccc6c5)cc34)nc(-c3ccc4oc5ccccc5c4c3)n2)cc1 ; c1ccc(-c2nc(-c3ccc4c(c3)oc3ccc(-c5ccc6ccccc6c5)cc34)nc(-c3cccc4oc5ccccc5c34)n2)cc1]

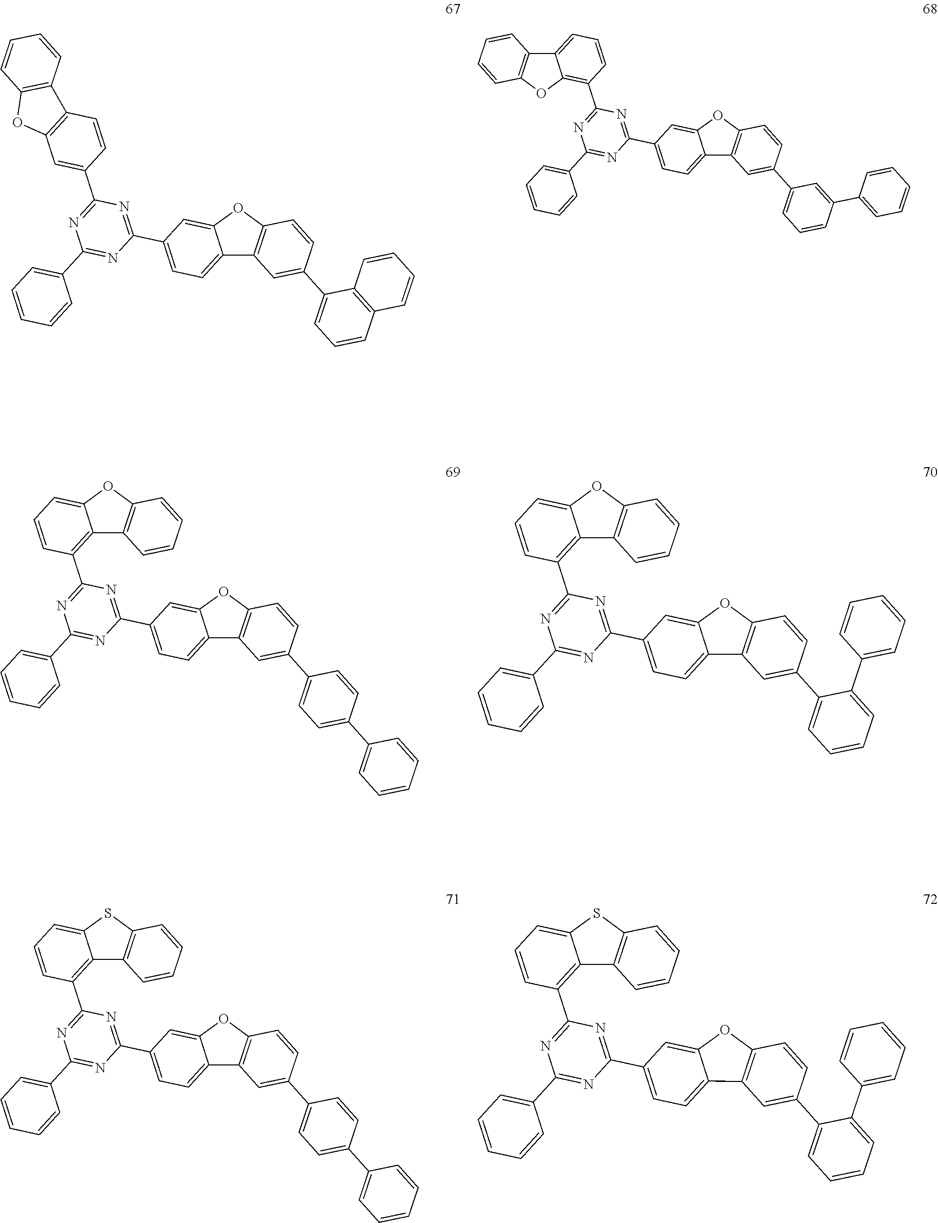

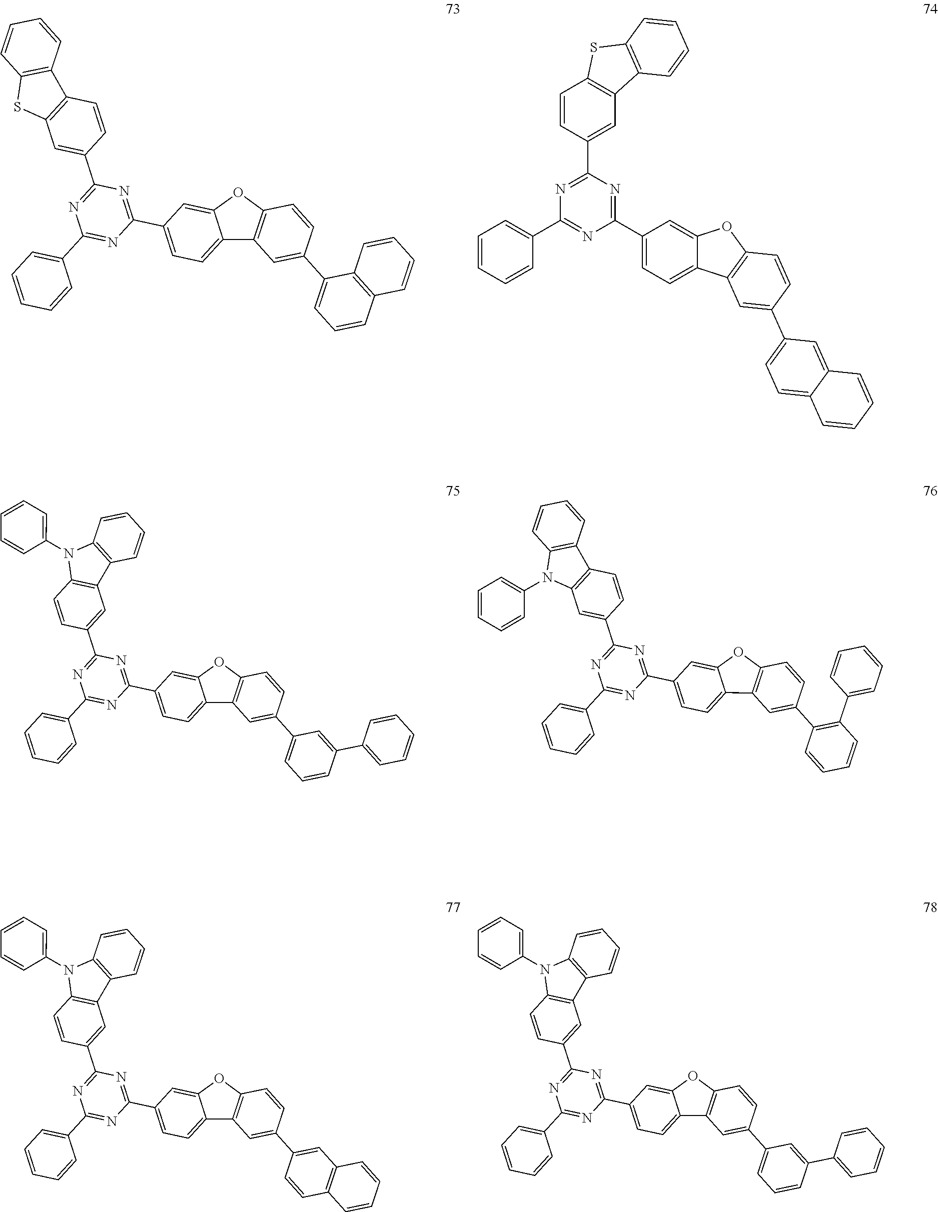

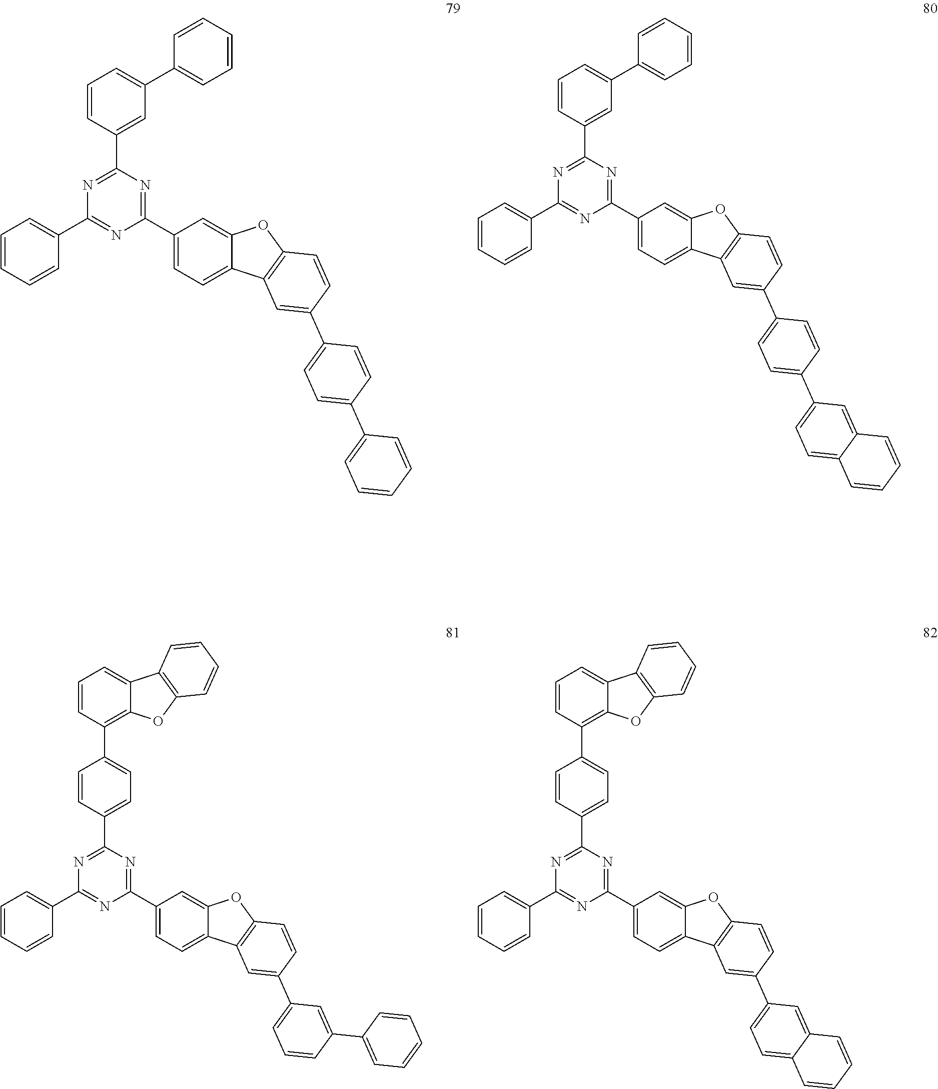 [SMILES: c1ccc(-c2ccc(-c3ccc4oc5cc(-c6nc(-c7ccccc7)nc(-c7cccc(-c8ccccc8)c7)n6)ccc5c4c3)cc2)cc1 ; c1ccc(-c2cccc(-c3nc(-c4ccccc4)nc(-c4ccc5c(c4)oc4ccc(-c6ccc(-c7ccc8ccccc8c7)cc6)cc45)n3)c2)cc1 ; c1ccc(-c2cccc(-c3ccc4oc5cc(-c6nc(-c7ccccc7)nc(-c7ccc(-c8cccc9c8oc8ccccc89)cc7)n6)ccc5c4c3)c2)cc1 ; c1ccc(-c2nc(-c3ccc(-c4cccc5c4oc4ccccc45)cc3)nc(-c3ccc4c(c3)oc3ccc(-c5ccc6ccccc6c5)cc34)n2)cc1]

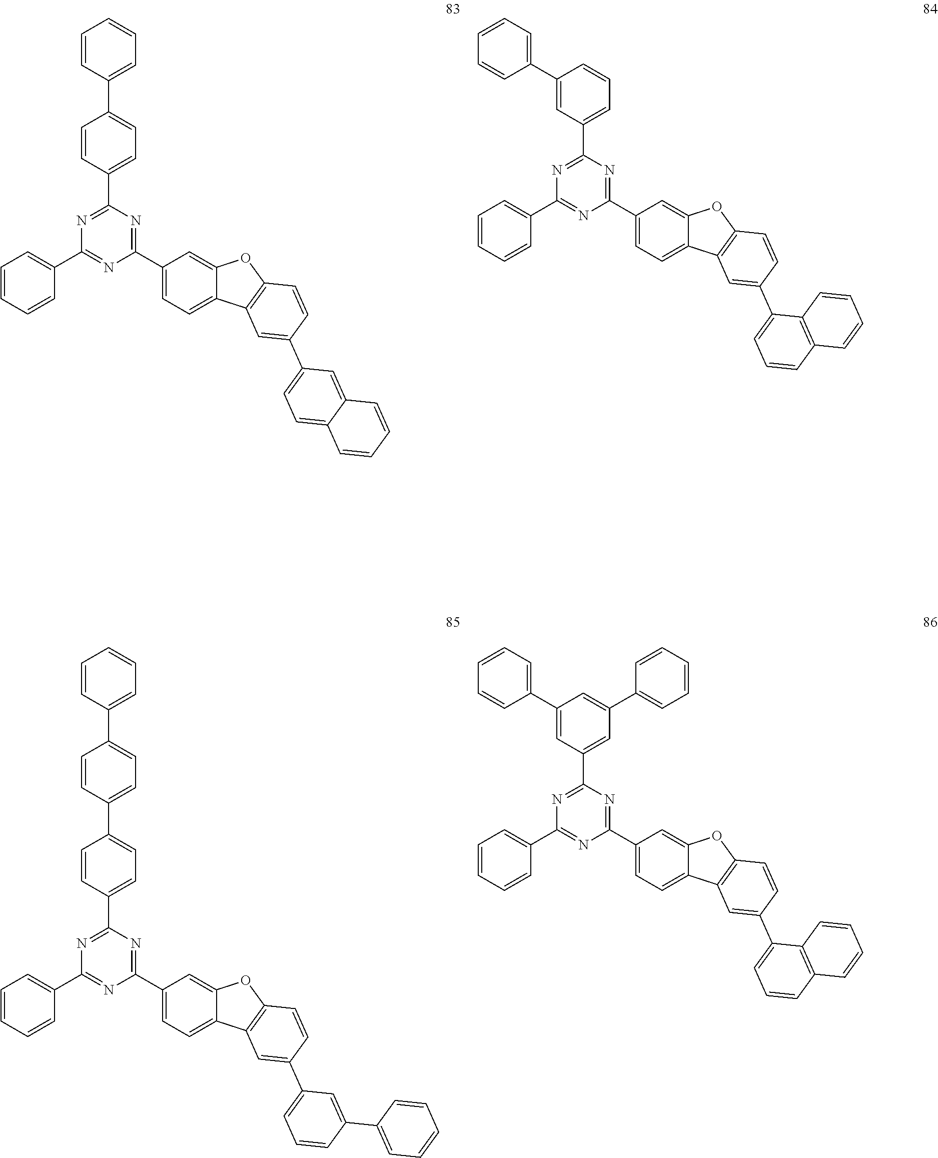 [SMILES: c1ccc(-c2ccc(-c3nc(-c4ccccc4)nc(-c4ccc5c(c4)oc4ccc(-c6ccc7ccccc7c6)cc45)n3)cc2)cc1 ; c1ccc(-c2cccc(-c3nc(-c4ccccc4)nc(-c4ccc5c(c4)oc4ccc(-c6cccc7ccccc67)cc45)n3)c2)cc1 ; c1ccc(-c2ccc(-c3ccc(-c4nc(-c5ccccc5)nc(-c5ccc6c(c5)oc5ccc(-c7cccc(-c8ccccc8)c7)cc56)n4)cc3)cc2)cc1 ; c1ccc(-c2cc(-c3ccccc3)cc(-c3nc(-c4ccccc4)nc(-c4ccc5c(c4)oc4ccc(-c6cccc7ccccc67)cc45)n3)c2)cc1]

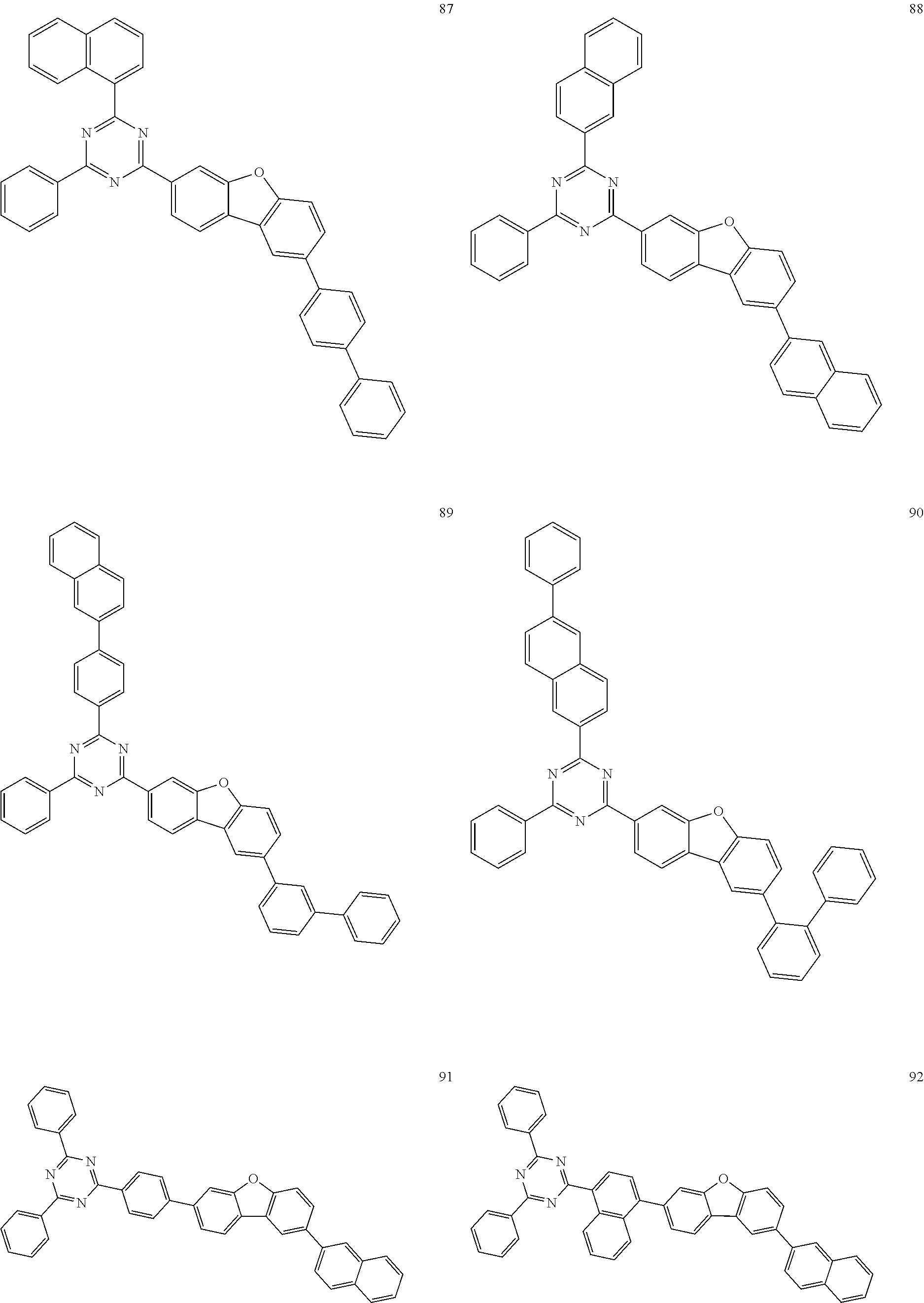

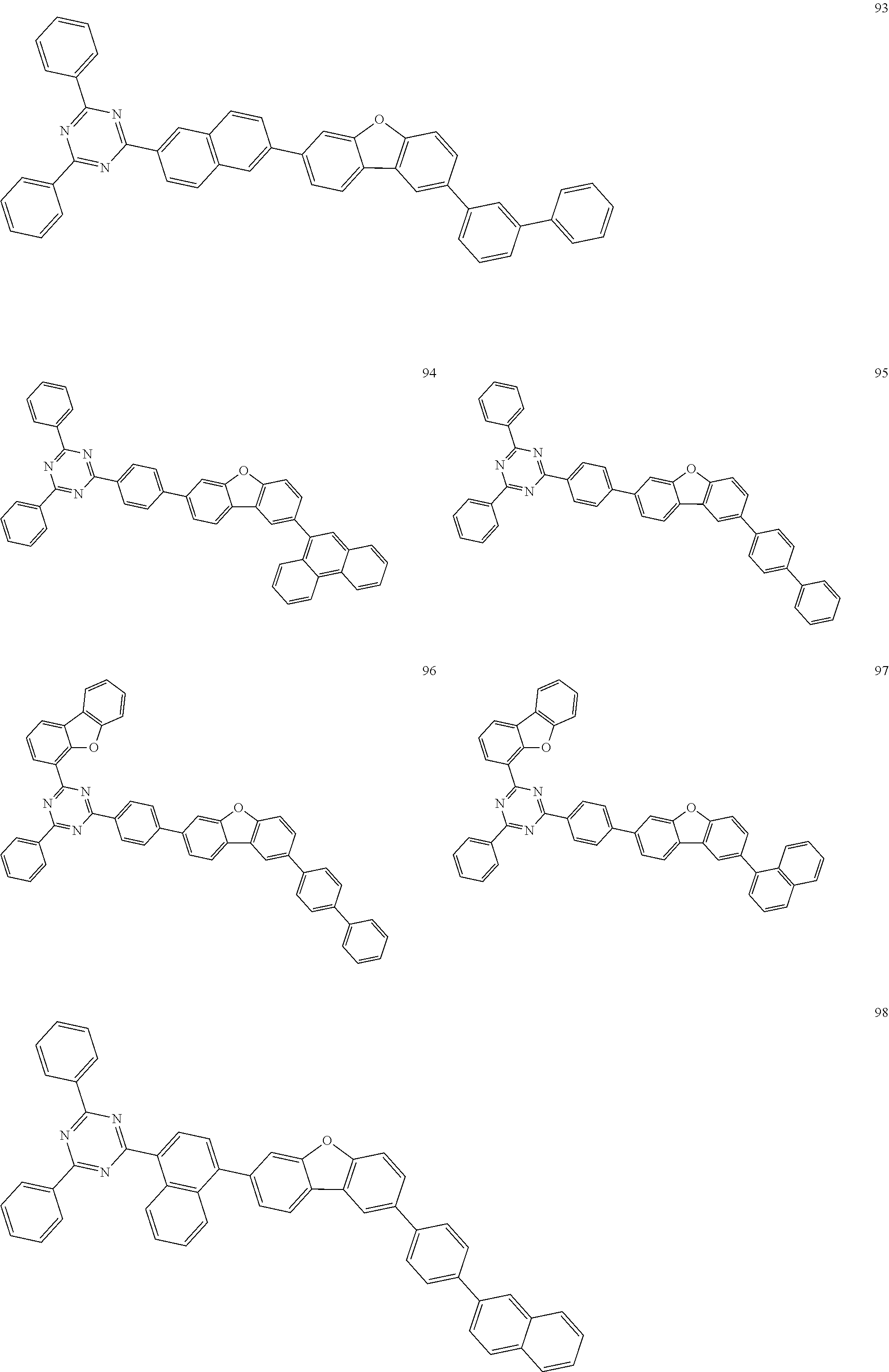

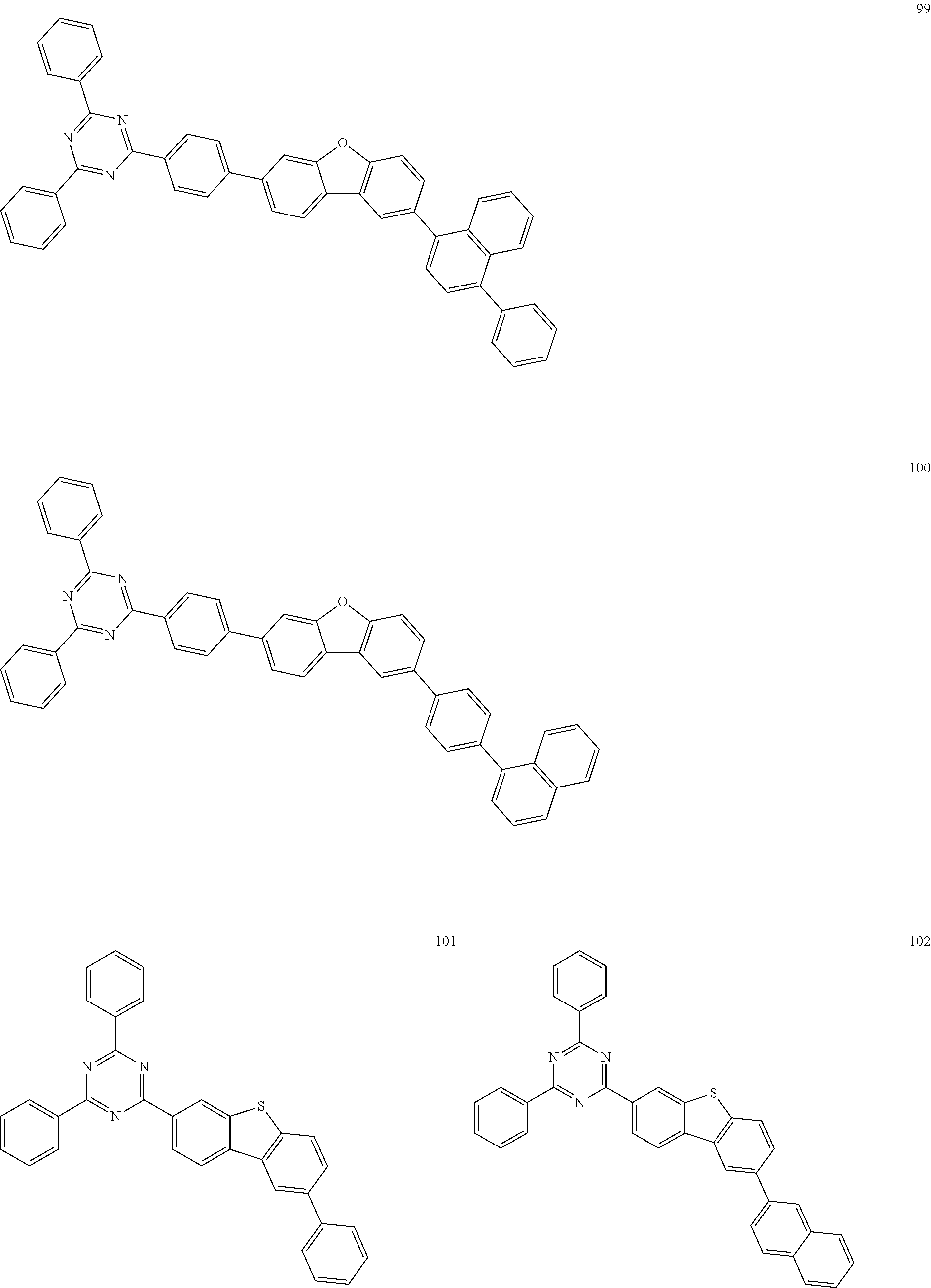 [SMILES: c1ccc(-c2nc(-c3ccccc3)nc(-c3ccc(-c4ccc5c(c4)oc4ccc(-c6ccc(-c7ccccc7)c7ccccc67)cc45)cc3)n2)cc1 ; c1ccc(-c2nc(-c3ccccc3)nc(-c3ccc(-c4ccc5c(c4)oc4ccc(-c6ccc(-c7cccc8ccccc78)cc6)cc45)cc3)n2)cc1 ; c1ccc(-c2ccc3sc4cc(-c5nc(-c6ccccc6)nc(-c6ccccc6)n5)ccc4c3c2)cc1 ; c1ccc(-c2nc(-c3ccccc3)nc(-c3ccc4c(c3)sc3ccc(-c5ccc6ccccc6c5)cc34)n2)cc1]

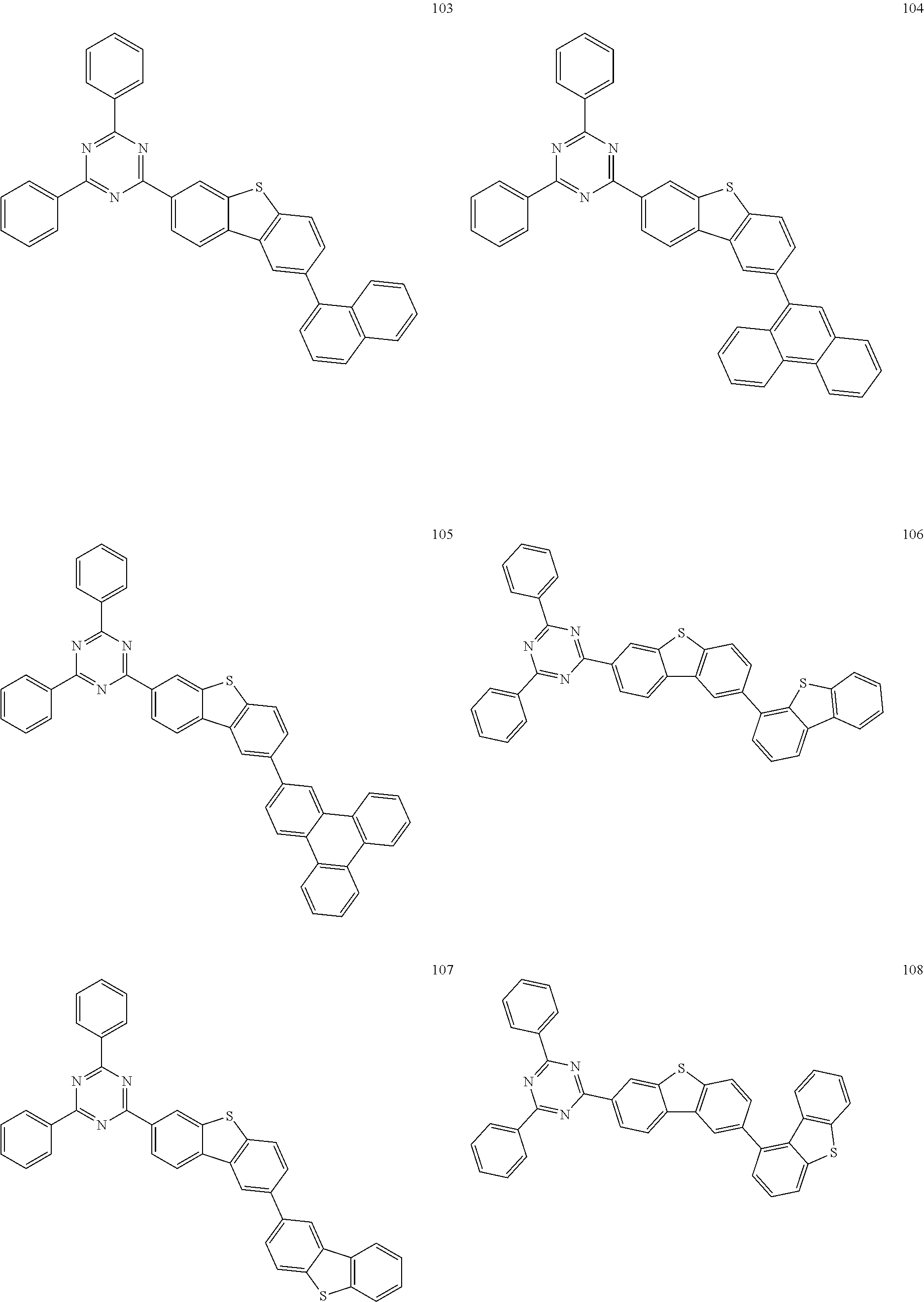 [SMILES: c1ccc(-c2nc(-c3ccccc3)nc(-c3ccc4c(c3)sc3ccc(-c5cccc6ccccc56)cc34)n2)cc1 ; c1ccc(-c2nc(-c3ccccc3)nc(-c3ccc4c(c3)sc3ccc(-c5cc6ccccc6c6ccccc56)cc34)n2)cc1 ; c1ccc(-c2nc(-c3ccccc3)nc(-c3ccc4c(c3)sc3ccc(-c5ccc6c7ccccc7c7ccccc7c6c5)cc34)n2)cc1 ; c1ccc(-c2nc(-c3ccccc3)nc(-c3ccc4c(c3)sc3ccc(-c5cccc6c5sc5ccccc56)cc34)n2)cc1 ; c1ccc(-c2nc(-c3ccccc3)nc(-c3ccc4c(c3)sc3ccc(-c5ccc6sc7ccccc7c6c5)cc34)n2)cc1 ; c1ccc(-c2nc(-c3ccccc3)nc(-c3ccc4c(c3)sc3ccc(-c5cccc6sc7ccccc7c56)cc34)n2)cc1]

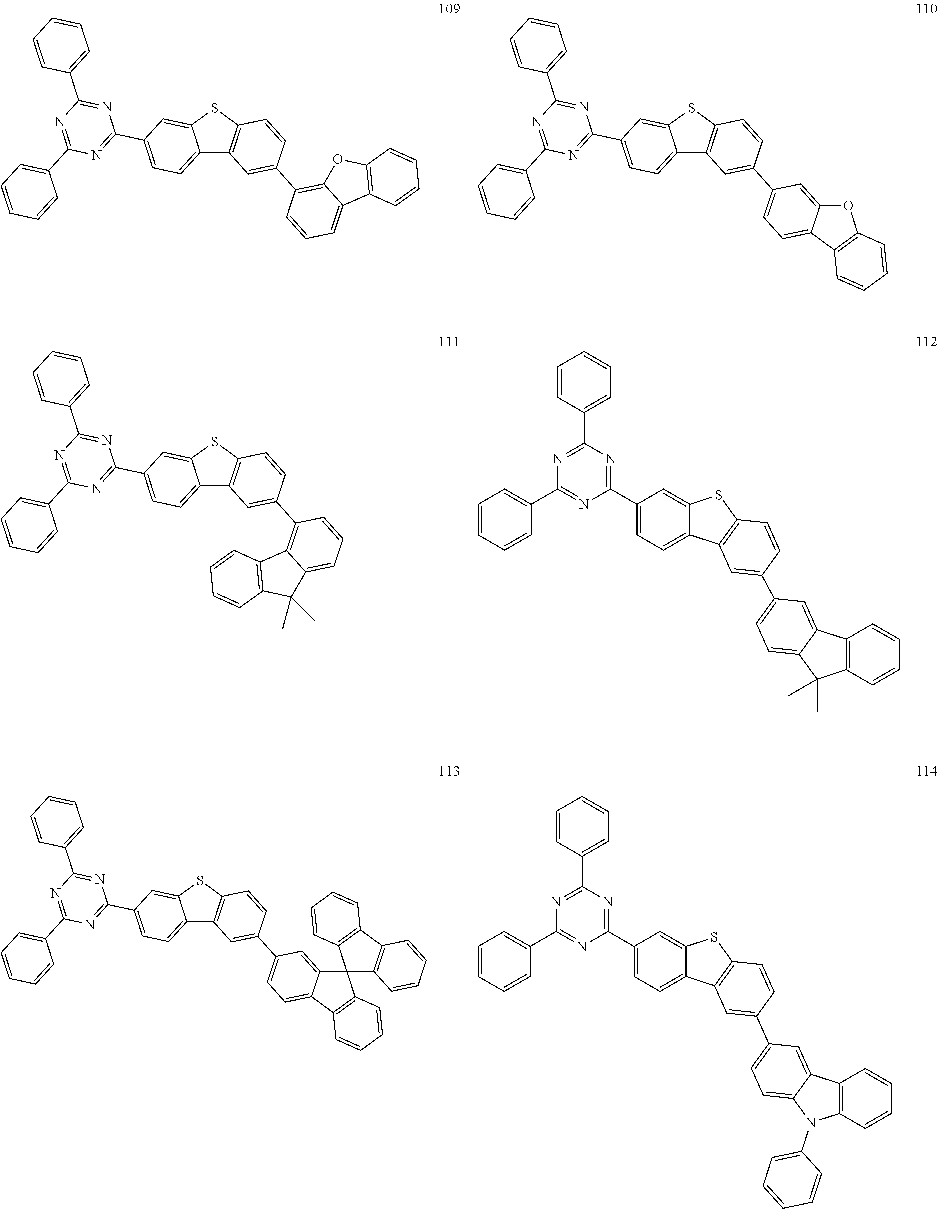

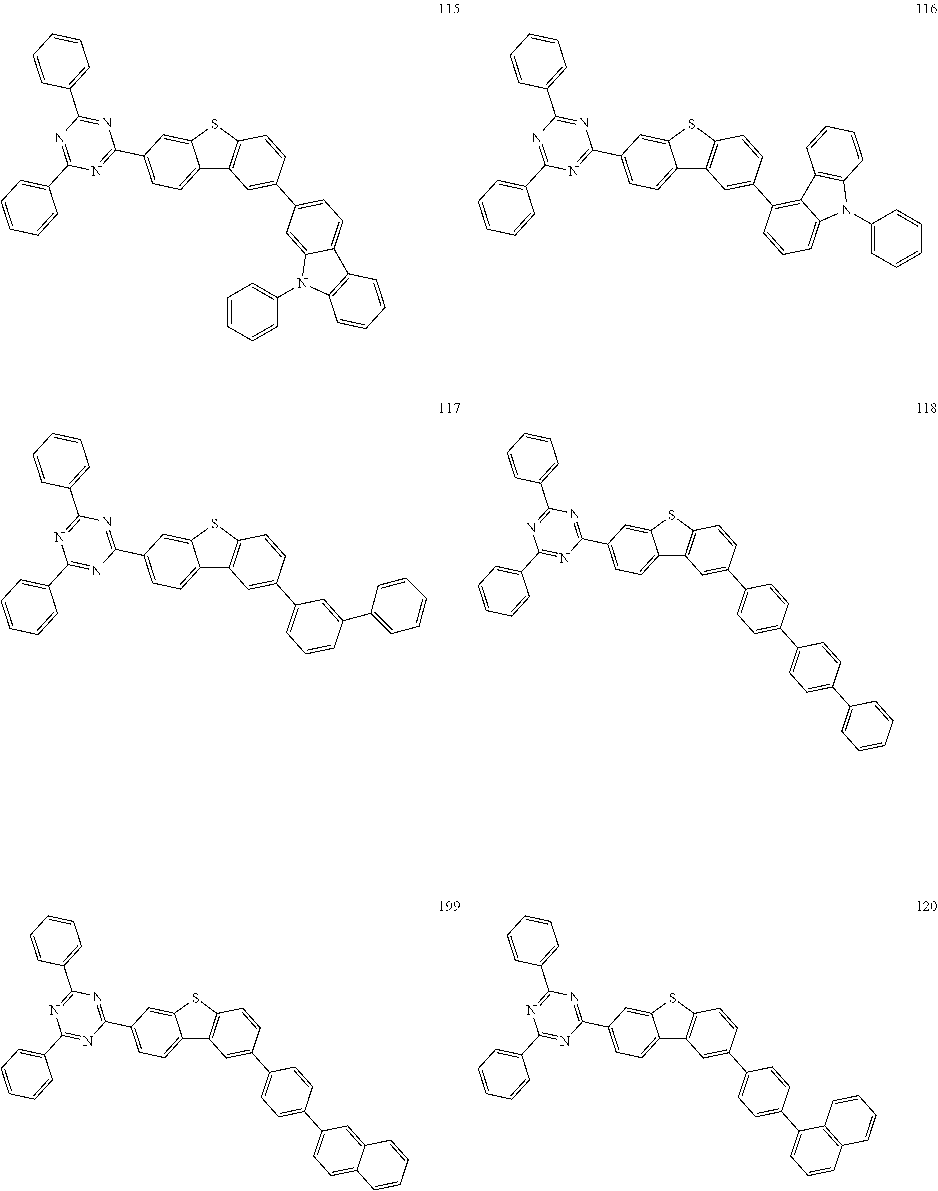

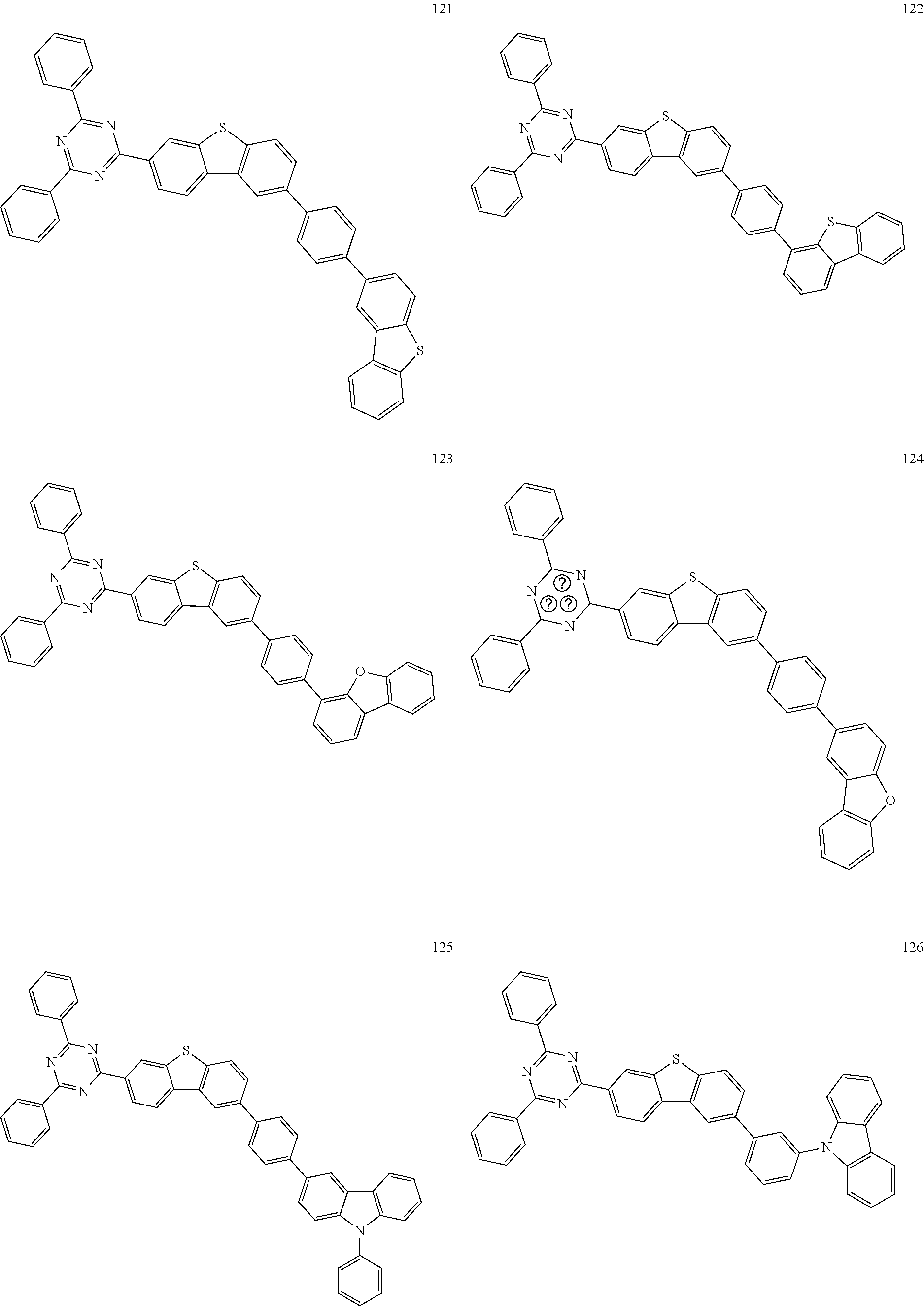

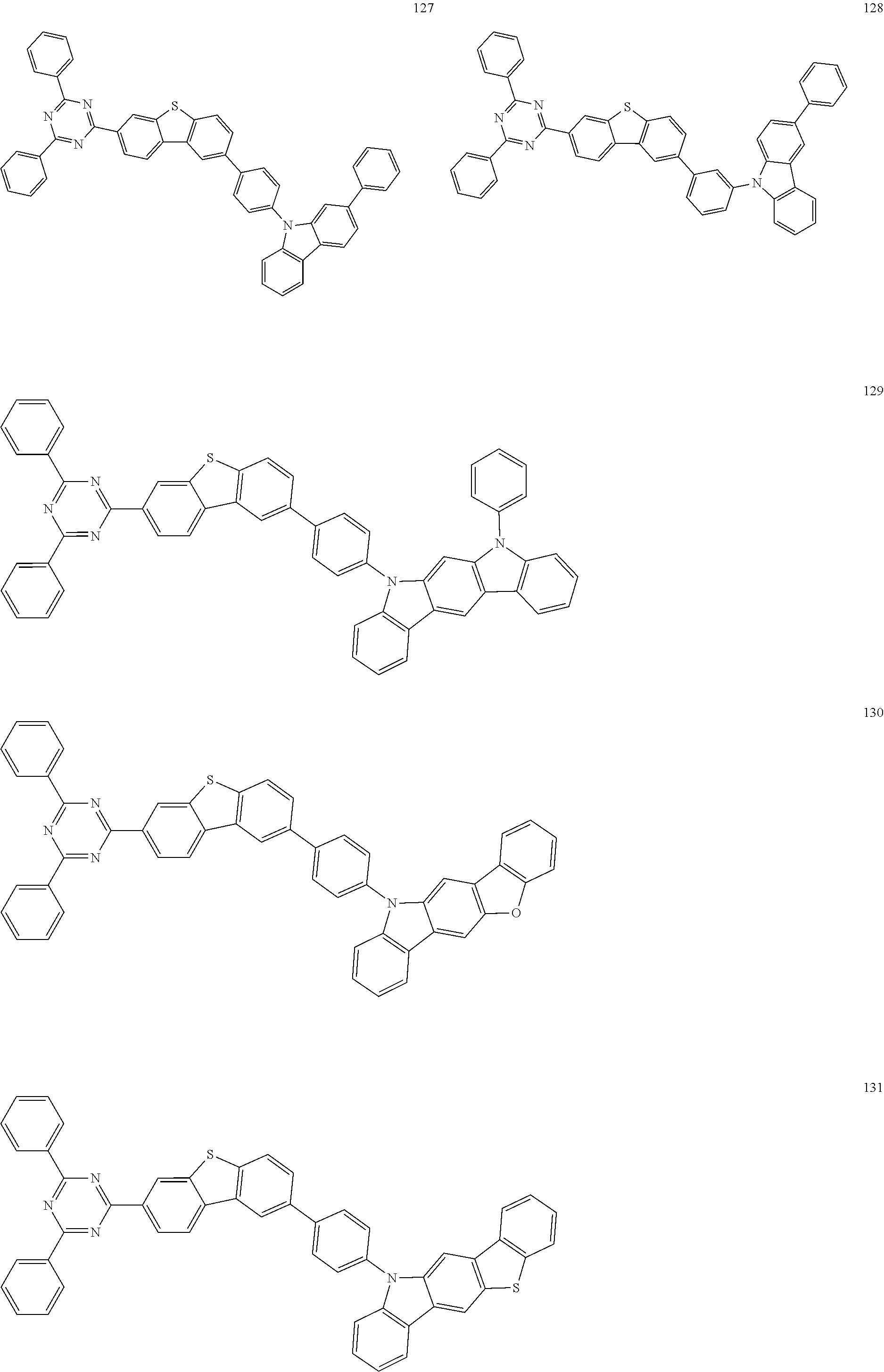

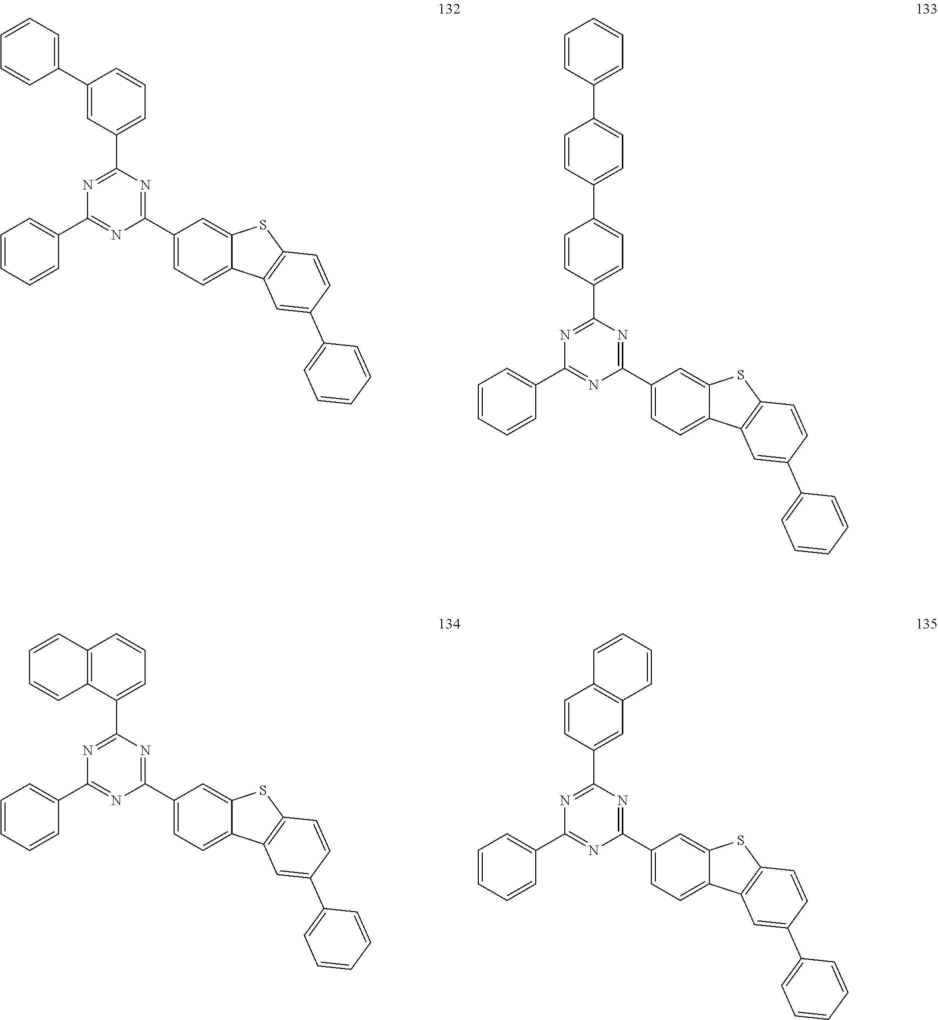 [SMILES: c1ccc(-c2cccc(-c3nc(-c4ccccc4)nc(-c4ccc5c(c4)sc4ccc(-c6ccccc6)cc45)n3)c2)cc1 ; c1ccc(-c2ccc(-c3ccc(-c4nc(-c5ccccc5)nc(-c5ccc6c(c5)sc5ccc(-c7ccccc7)cc56)n4)cc3)cc2)cc1 ; c1ccc(-c2ccc3sc4cc(-c5nc(-c6ccccc6)nc(-c6cccc7ccccc67)n5)ccc4c3c2)cc1 ; c1ccc(-c2ccc3sc4cc(-c5nc(-c6ccccc6)nc(-c6ccc7ccccc7c6)n5)ccc4c3c2)cc1]

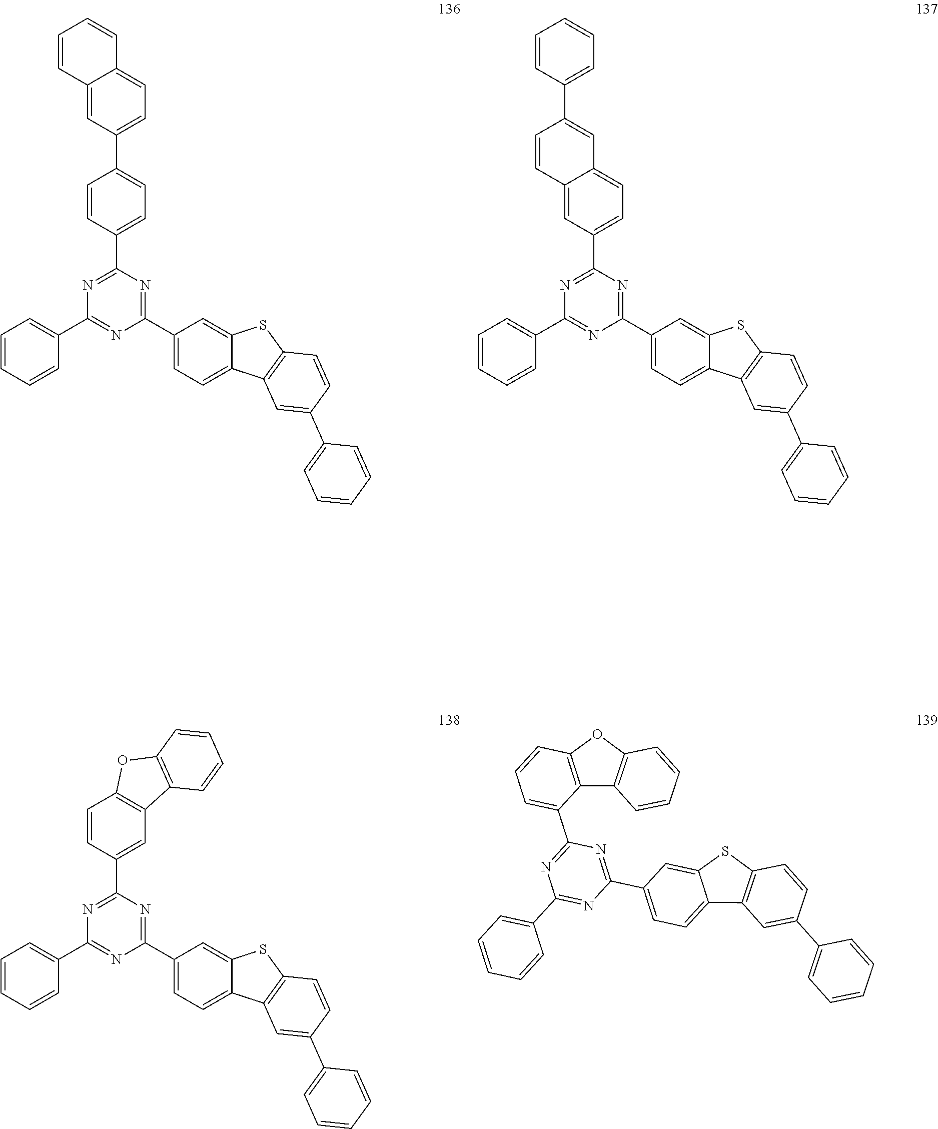 [SMILES: c1ccc(-c2ccc3sc4cc(-c5nc(-c6ccccc6)nc(-c6ccc(-c7ccc8ccccc8c7)cc6)n5)ccc4c3c2)cc1 ; c1ccc(-c2ccc3cc(-c4nc(-c5ccccc5)nc(-c5ccc6c(c5)sc5ccc(-c7ccccc7)cc56)n4)ccc3c2)cc1 ; c1ccc(-c2ccc3sc4cc(-c5nc(-c6ccccc6)nc(-c6ccc7oc8ccccc8c7c6)n5)ccc4c3c2)cc1 ; c1ccc(-c2ccc3sc4cc(-c5nc(-c6ccccc6)nc(-c6cccc7oc8ccccc8c67)n5)ccc4c3c2)cc1]

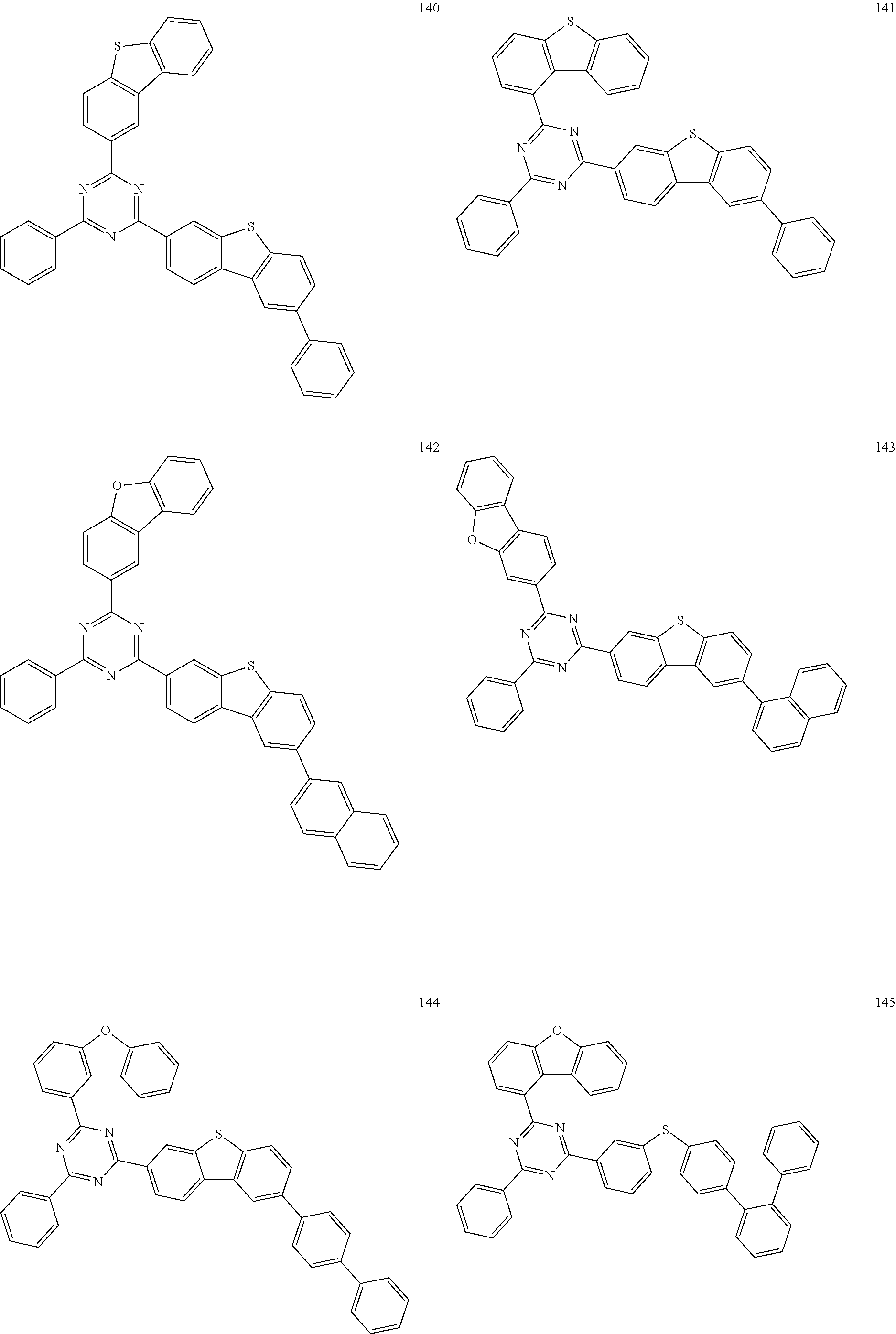 [SMILES: c1ccc(-c2ccc3sc4cc(-c5nc(-c6ccccc6)nc(-c6ccc7sc8ccccc8c7c6)n5)ccc4c3c2)cc1 ; c1ccc(-c2ccc3sc4cc(-c5nc(-c6ccccc6)nc(-c6cccc7sc8ccccc8c67)n5)ccc4c3c2)cc1 ; c1ccc(-c2nc(-c3ccc4c(c3)sc3ccc(-c5ccc6ccccc6c5)cc34)nc(-c3ccc4oc5ccccc5c4c3)n2)cc1 ; c1ccc(-c2nc(-c3ccc4c(c3)oc3ccccc34)nc(-c3ccc4c(c3)sc3ccc(-c5cccc6ccccc56)cc34)n2)cc1 ; c1ccc(-c2ccc(-c3ccc4sc5cc(-c6nc(-c7ccccc7)nc(-c7cccc8oc9ccccc9c78)n6)ccc5c4c3)cc2)cc1 ; c1ccc(-c2nc(-c3ccc4c(c3)sc3ccc(-c5ccccc5-c5ccccc5)cc34)nc(-c3cccc4oc5ccccc5c34)n2)cc1]

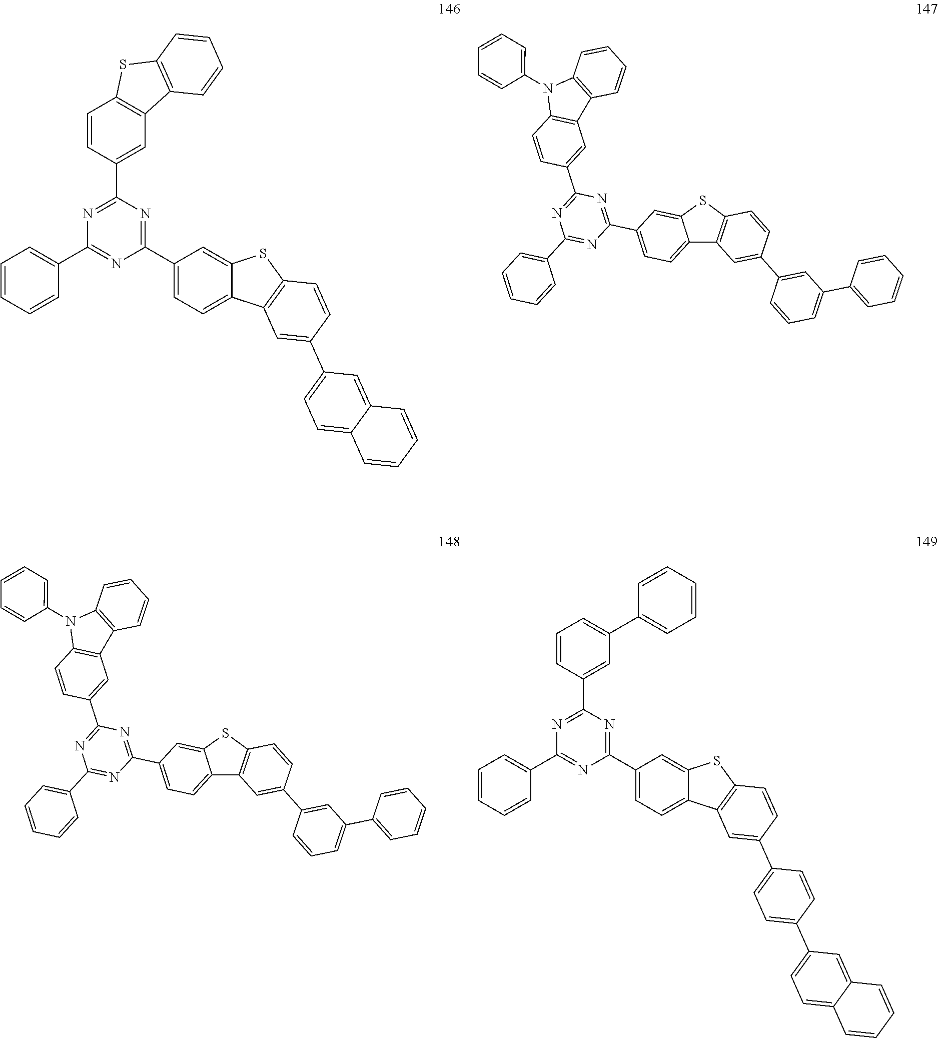 [SMILES: c1ccc(-c2nc(-c3ccc4c(c3)sc3ccc(-c5ccc6ccccc6c5)cc34)nc(-c3ccc4sc5ccccc5c4c3)n2)cc1 ; c1ccc(-c2cccc(-c3ccc4sc5cc(-c6nc(-c7ccccc7)nc(-c7ccc8c(c7)c7ccccc7n8-c7ccccc7)n6)ccc5c4c3)c2)cc1 ; c1ccc(-c2cccc(-c3ccc4sc5cc(-c6nc(-c7ccccc7)nc(-c7ccc8c(c7)c7ccccc7n8-c7ccccc7)n6)ccc5c4c3)c2)cc1 ; c1ccc(-c2cccc(-c3nc(-c4ccccc4)nc(-c4ccc5c(c4)sc4ccc(-c6ccc(-c7ccc8ccccc8c7)cc6)cc45)n3)c2)cc1]

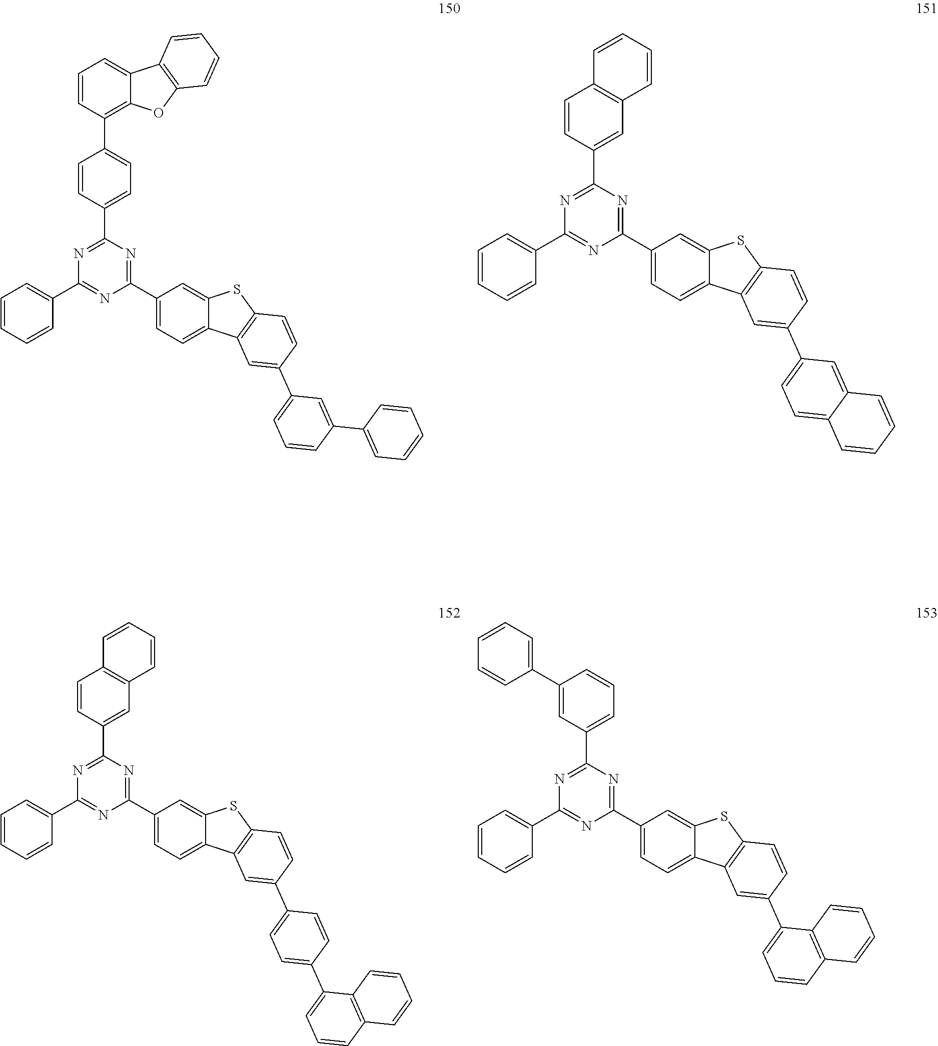 [SMILES: c1ccc(-c2cccc(-c3ccc4sc5cc(-c6nc(-c7ccccc7)nc(-c7ccc(-c8cccc9c8oc8ccccc89)cc7)n6)ccc5c4c3)c2)cc1 ; c1ccc(-c2nc(-c3ccc4ccccc4c3)nc(-c3ccc4c(c3)sc3ccc(-c5ccc6ccccc6c5)cc34)n2)cc1 ; c1ccc(-c2nc(-c3ccc4ccccc4c3)nc(-c3ccc4c(c3)sc3ccc(-c5ccc(-c6cccc7ccccc67)cc5)cc34)n2)cc1 ; c1ccc(-c2cccc(-c3nc(-c4ccccc4)nc(-c4ccc5c(c4)sc4ccc(-c6cccc7ccccc67)cc45)n3)c2)cc1]

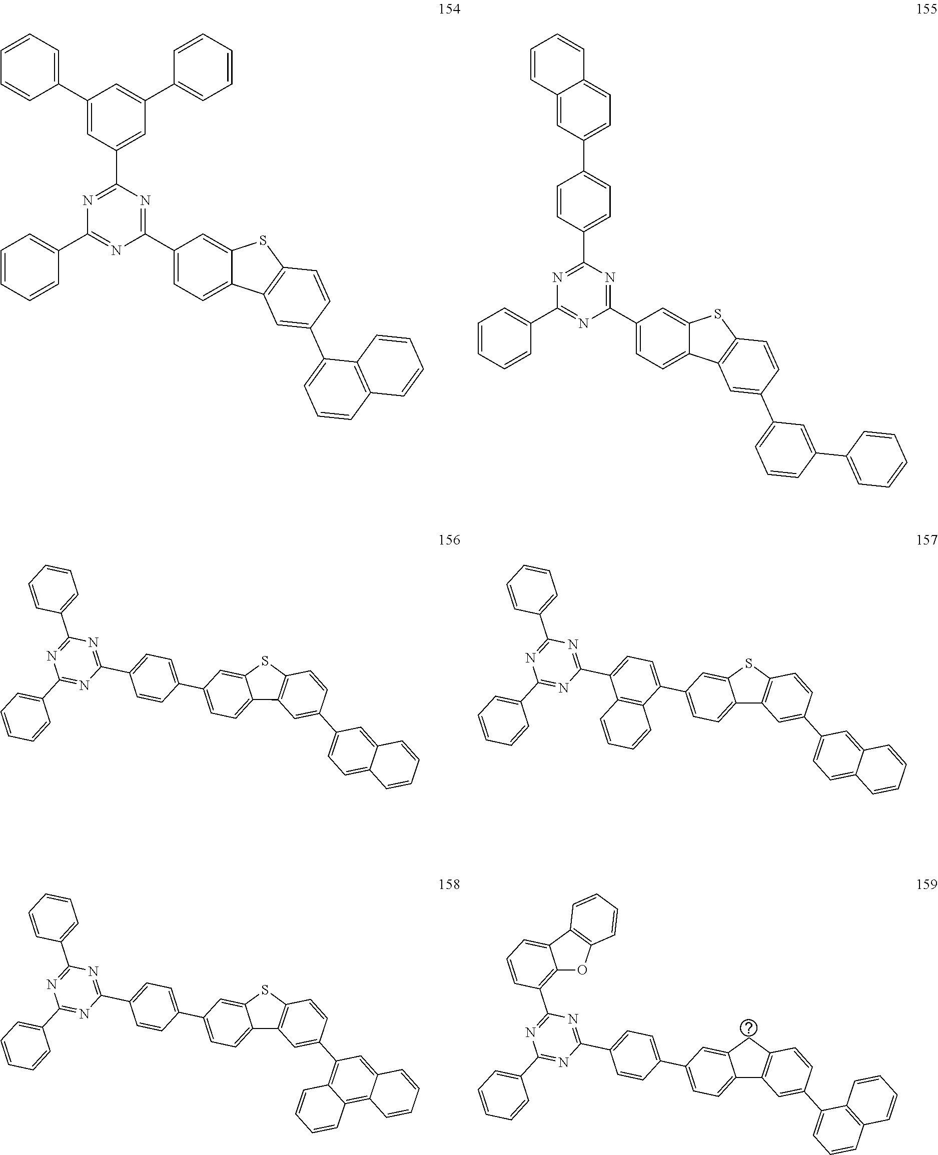

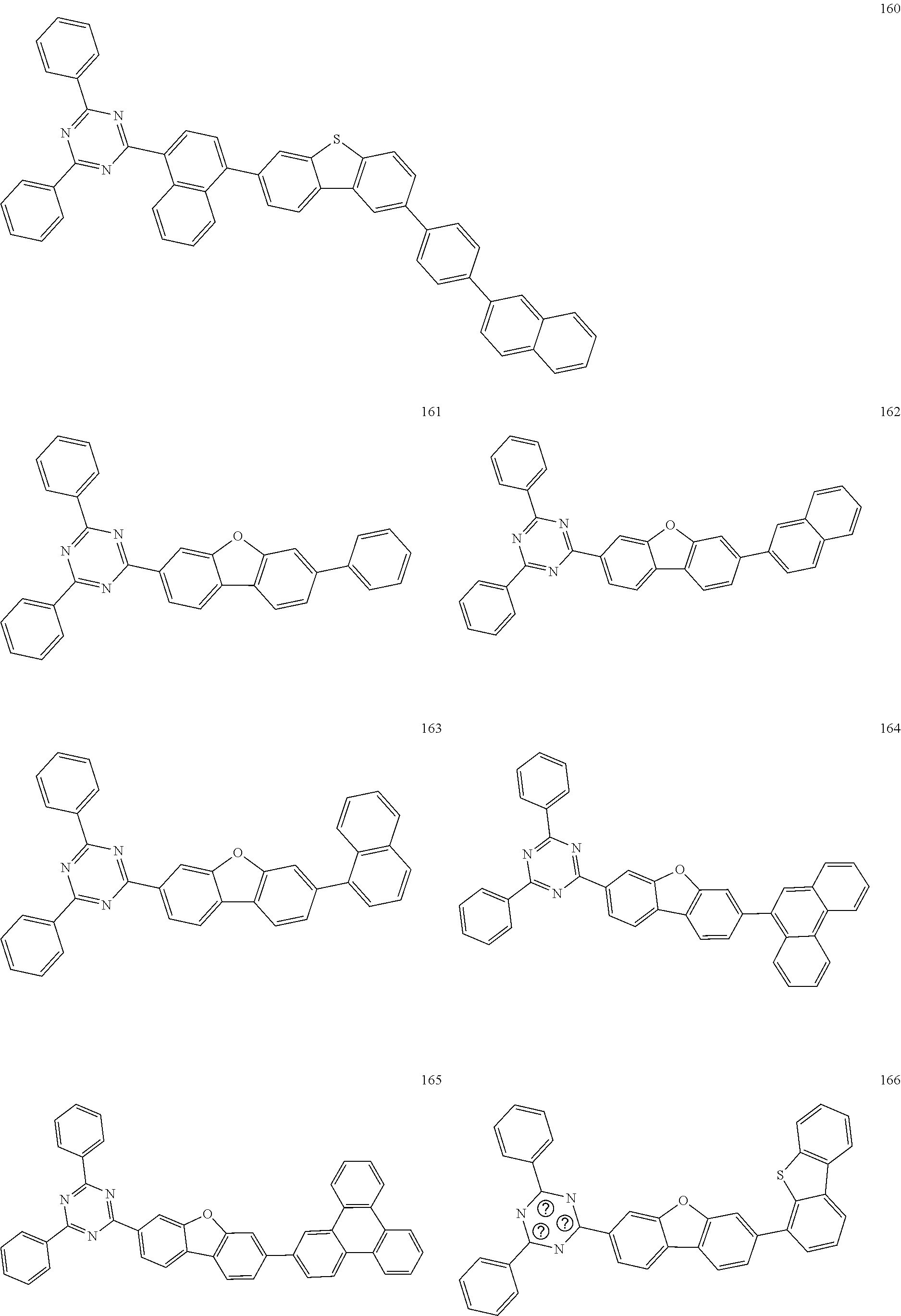

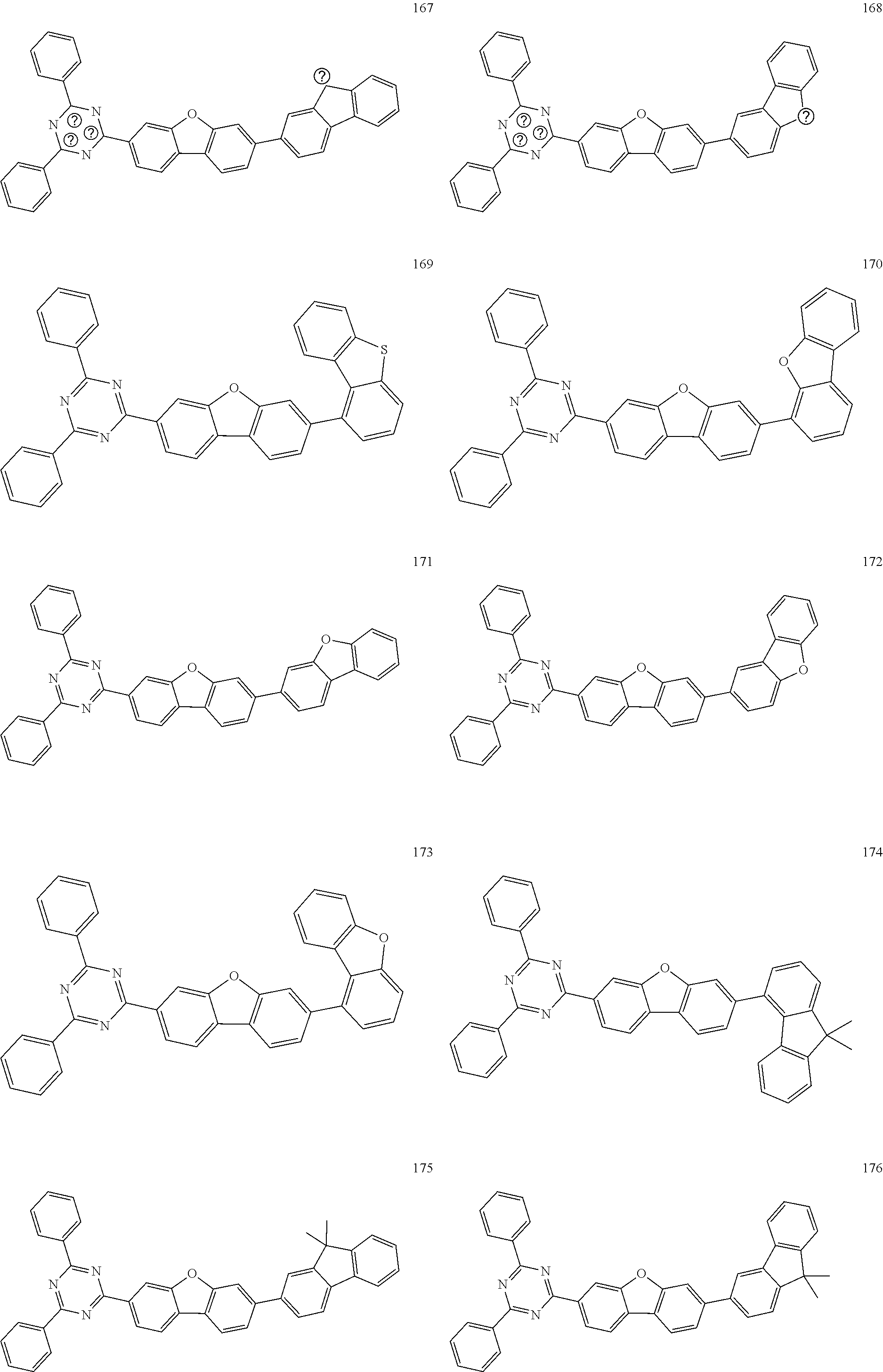

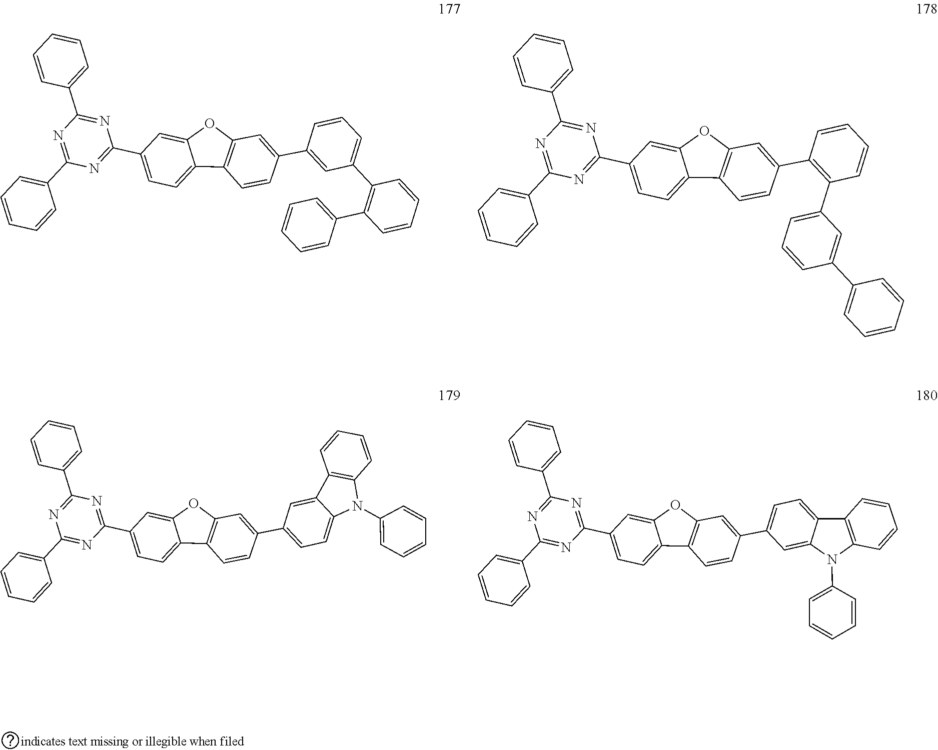 [SMILES: c1ccc(-c2nc(-c3ccccc3)nc(-c3ccc4c(c3)oc3cc(-c5cccc(-c6ccccc6-c6ccccc6)c5)ccc34)n2)cc1 ; c1ccc(-c2cccc(-c3ccccc3-c3ccc4c(c3)oc3cc(-c5nc(-c6ccccc6)nc(-c6ccccc6)n5)ccc34)c2)cc1 ; c1ccc(-c2nc(-c3ccccc3)nc(-c3ccc4c(c3)oc3cc(-c5ccc6c(c5)c5ccccc5n6-c5ccccc5)ccc34)n2)cc1 ; c1ccc(-c2nc(-c3ccccc3)nc(-c3ccc4c(c3)oc3cc(-c5ccc6c7ccccc7n(-c7ccccc7)c6c5)ccc34)n2)cc1]

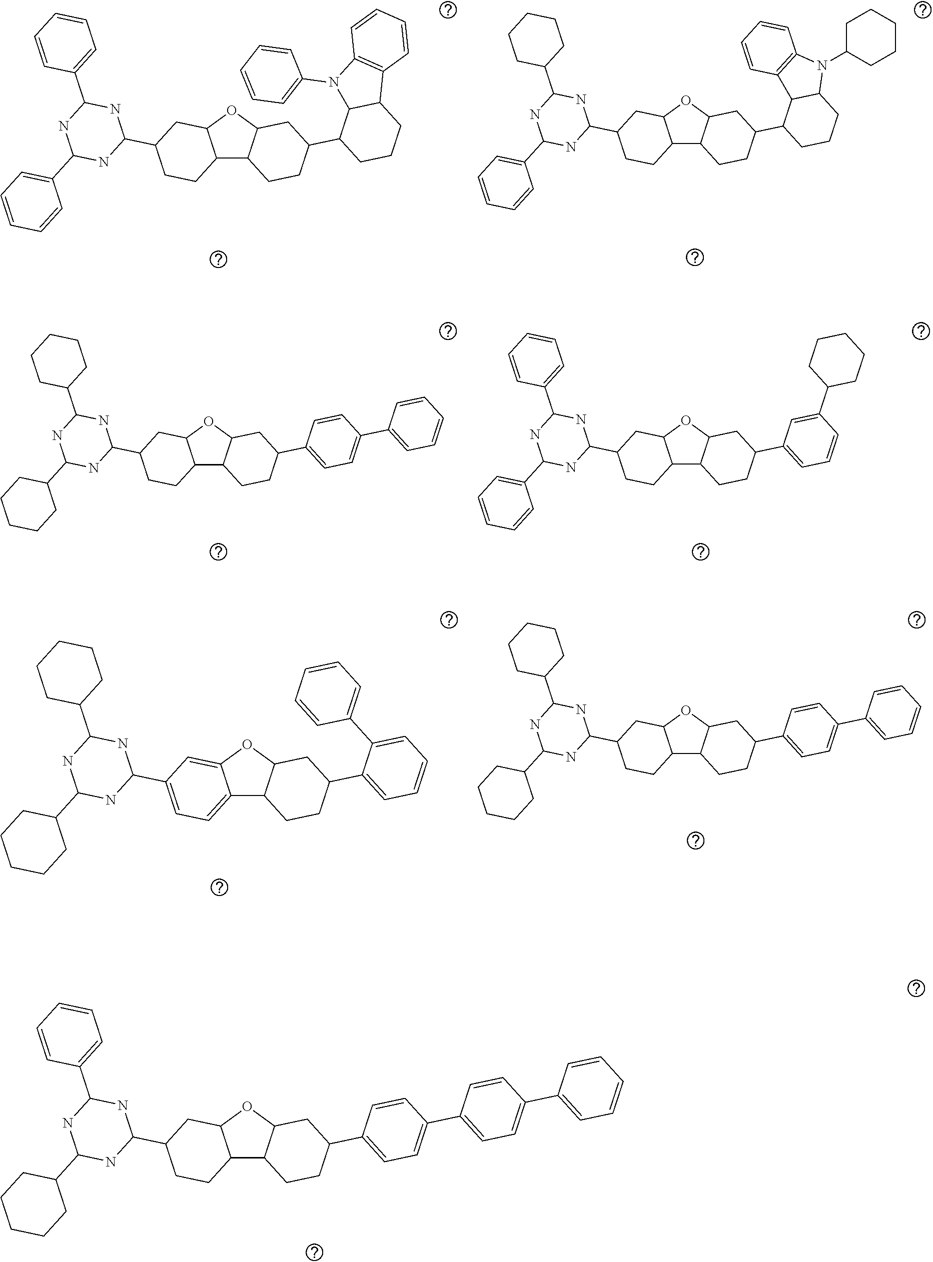

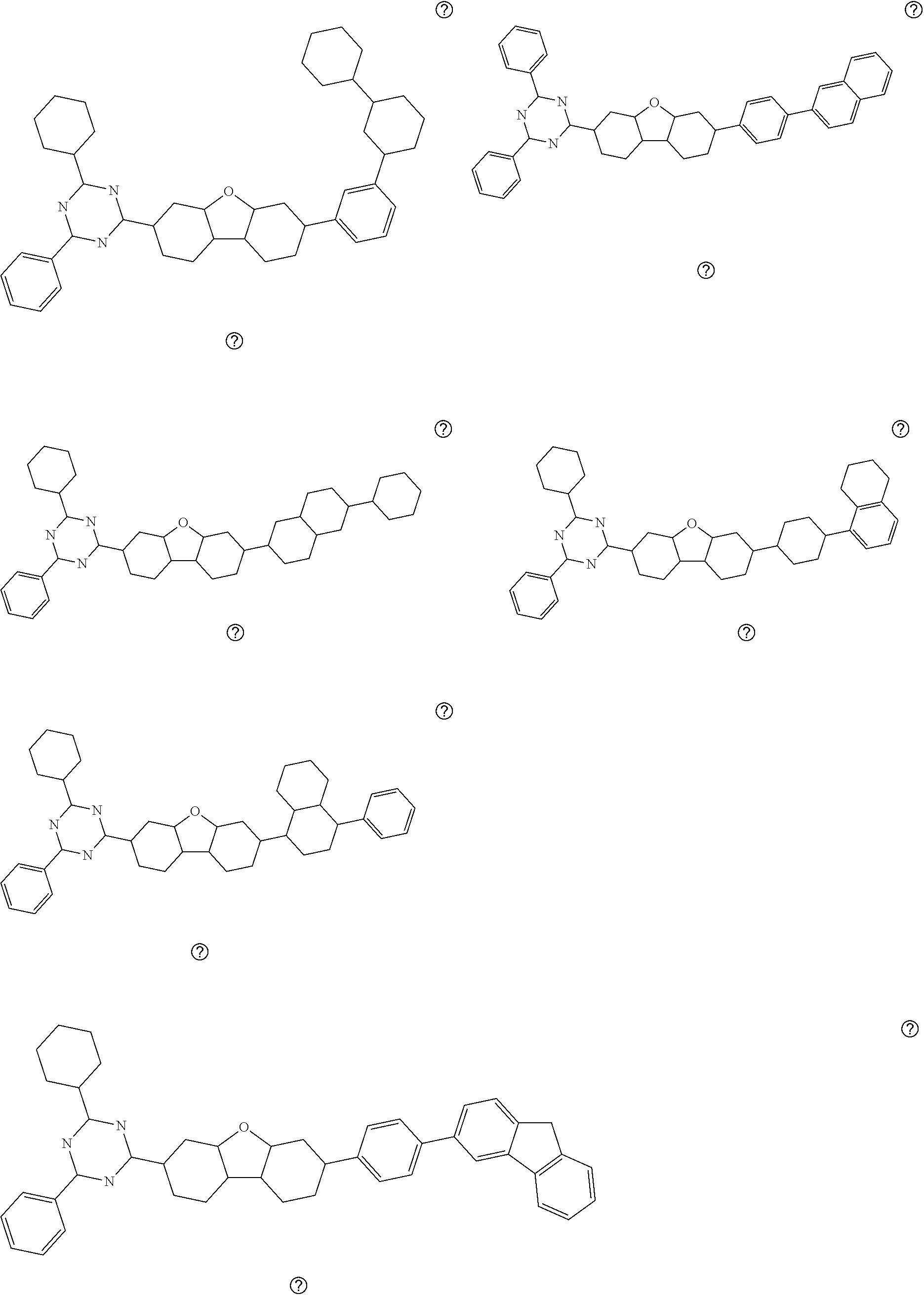 [SMILES: c1ccc(C2NC(C3CCCCC3)NC(C3CCC4C(C3)OC3CC(c5cccc(C6CCCC(C7CCCCC7)C6)c5)CCC34)N2)cc1 ; c1ccc(C2NC(c3ccccc3)NC(C3CCC4C(C3)OC3CC(c5ccc(-c6ccc7ccccc7c6)cc5)CCC34)N2)cc1 ; c1ccc(C2NC(C3CCCCC3)NC(C3CCC4C(C3)OC3CC(C5CCC6CC(C7CCCCC7)CCC6C5)CCC34)N2)cc1 ; c1ccc(C2NC(C3CCCCC3)NC(C3CCC4C(C3)OC3CC(C5CCC(c6cccc7c6CCCC7)CC5)CCC34)N2)cc1 ; c1ccc(C2NC(C3CCCCC3)NC(C3CCC4C(C3)OC3CC(C5CCC(c6ccccc6)C6CCCCC56)CCC34)N2)cc1 ; c1ccc(C2NC(C3CCCCC3)NC(C3CCC4C(C3)OC3CC(c5ccc(-c6ccc7c(c6)-c6ccccc6C7)cc5)CCC34)N2)cc1]

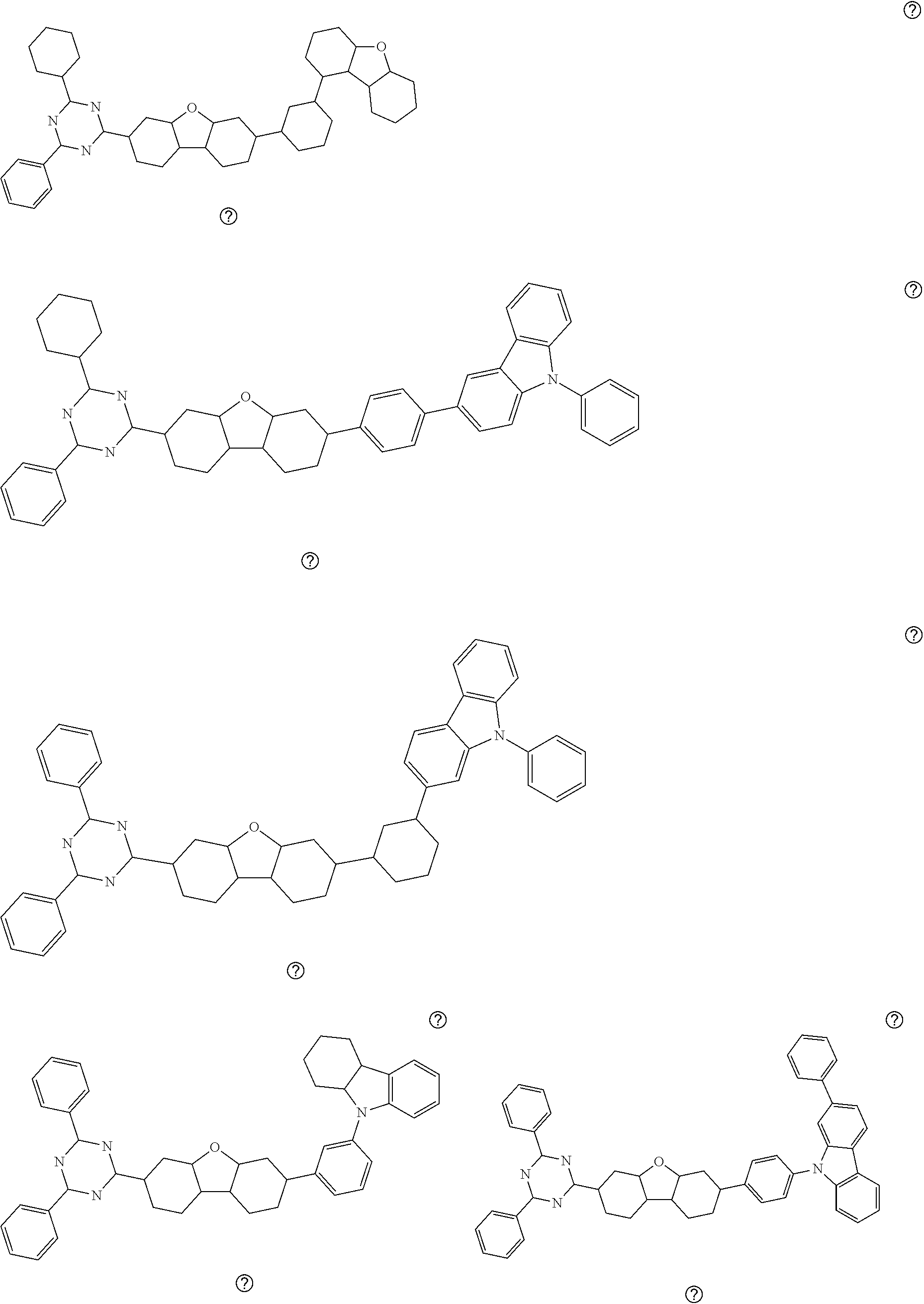 [SMILES: c1ccc(C2NC(C3CCCCC3)NC(C3CCC4C(C3)OC3CC(C5CCCC(C6CCCC7OC8CCCCC8C76)C5)CCC34)N2)cc1 ; c1ccc(C2NC(C3CCCCC3)NC(C3CCC4C(C3)OC3CC(c5ccc(-c6ccc7c(c6)c6ccccc6n7-c6ccccc6)cc5)CCC34)N2)cc1 ; c1ccc(C2NC(c3ccccc3)NC(C3CCC4C(C3)OC3CC(C5CCCC(c6ccc7c8ccccc8n(-c8ccccc8)c7c6)C5)CCC34)N2)cc1 ; c1ccc(C2NC(c3ccccc3)NC(C3CCC4C(C3)OC3CC(c5cccc(N6c7ccccc7C7CCCCC76)c5)CCC34)N2)cc1 ; c1ccc(-c2ccc3c4ccccc4n(-c4ccc(C5CCC6C(C5)OC5CC(C7NC(c8ccccc8)NC(c8ccccc8)N7)CCC56)cc4)c3c2)cc1]

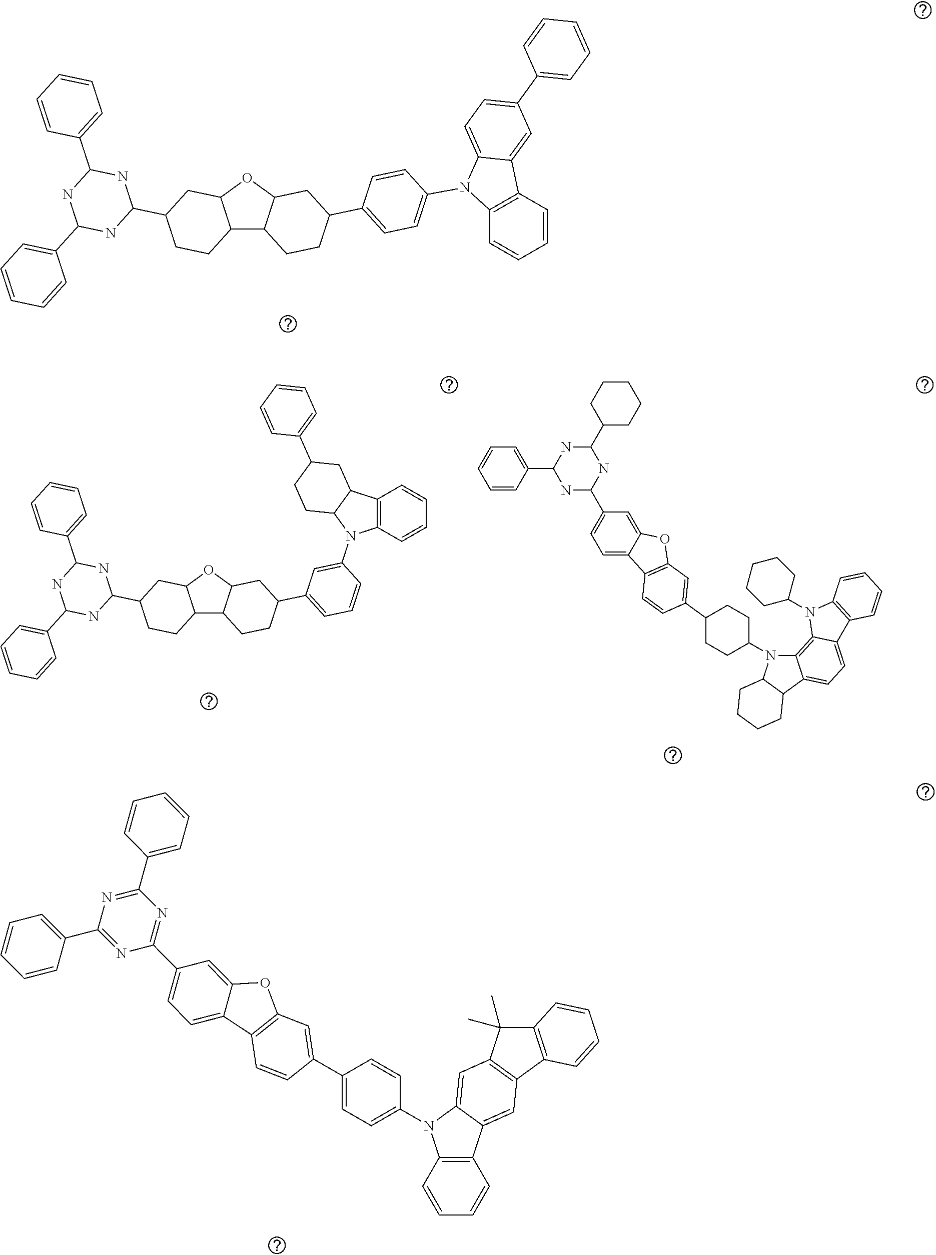 [SMILES: c1ccc(-c2ccc3c(c2)c2ccccc2n3-c2ccc(C3CCC4C(C3)OC3CC(C5NC(c6ccccc6)NC(c6ccccc6)N5)CCC34)cc2)cc1 ; c1ccc(C2CCC3C(C2)c2ccccc2N3c2cccc(C3CCC4C(C3)OC3CC(C5NC(c6ccccc6)NC(c6ccccc6)N5)CCC34)c2)cc1 ; c1ccc(C2NC(c3ccc4c(c3)oc3cc(C5CCC(N6c7c(ccc8c9ccccc9n(C9CCCCC9)c78)C7CCCCC76)CC5)ccc34)NC(C3CCCCC3)N2)cc1 ; CC1(C)c2ccccc2-c2cc3c4ccccc4n(-c4ccc(-c5ccc6c(c5)oc5cc(-c7nc(-c8ccccc8)nc(-c8ccccc8)n7)ccc56)cc4)c3cc21]

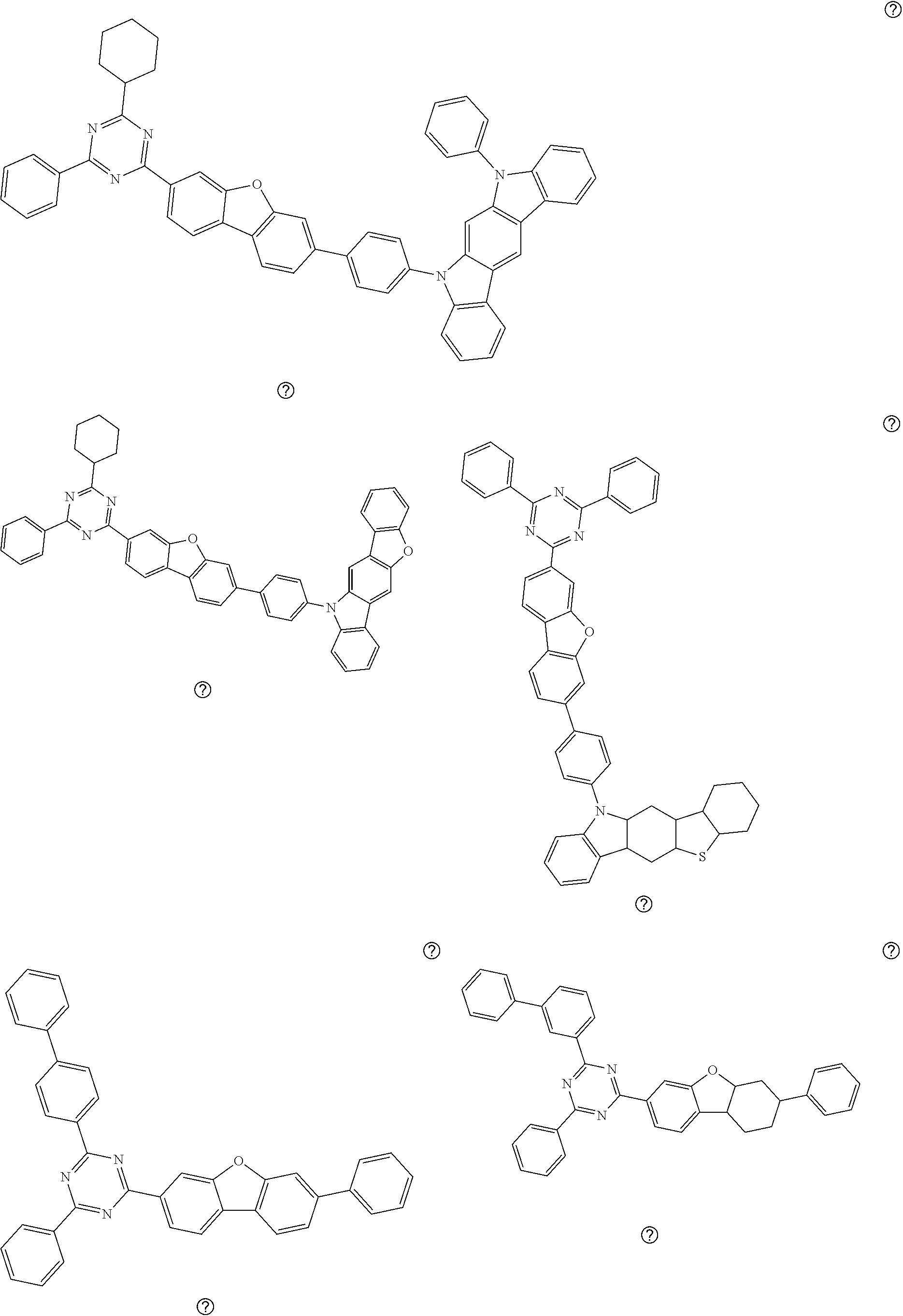 [SMILES: c1ccc(-c2nc(-c3ccc4c(c3)oc3cc(-c5ccc(-n6c7ccccc7c7cc8c9ccccc9n(-c9ccccc9)c8cc76)cc5)ccc34)nc(C3CCCCC3)n2)cc1 ; c1ccc(-c2nc(-c3ccc4c(c3)oc3cc(-c5ccc(-n6c7ccccc7c7cc8oc9ccccc9c8cc76)cc5)ccc34)nc(C3CCCCC3)n2)cc1 ; c1ccc(-c2nc(-c3ccccc3)nc(-c3ccc4c(c3)oc3cc(-c5ccc(N6c7ccccc7C7CC8SC9CCCCC9C8CC76)cc5)ccc34)n2)cc1 ; c1ccc(-c2ccc(-c3nc(-c4ccccc4)nc(-c4ccc5c(c4)oc4cc(-c6ccccc6)ccc45)n3)cc2)cc1 ; c1ccc(-c2cccc(-c3nc(-c4ccccc4)nc(-c4ccc5c(c4)OC4CC(c6ccccc6)CCC54)n3)c2)cc1]

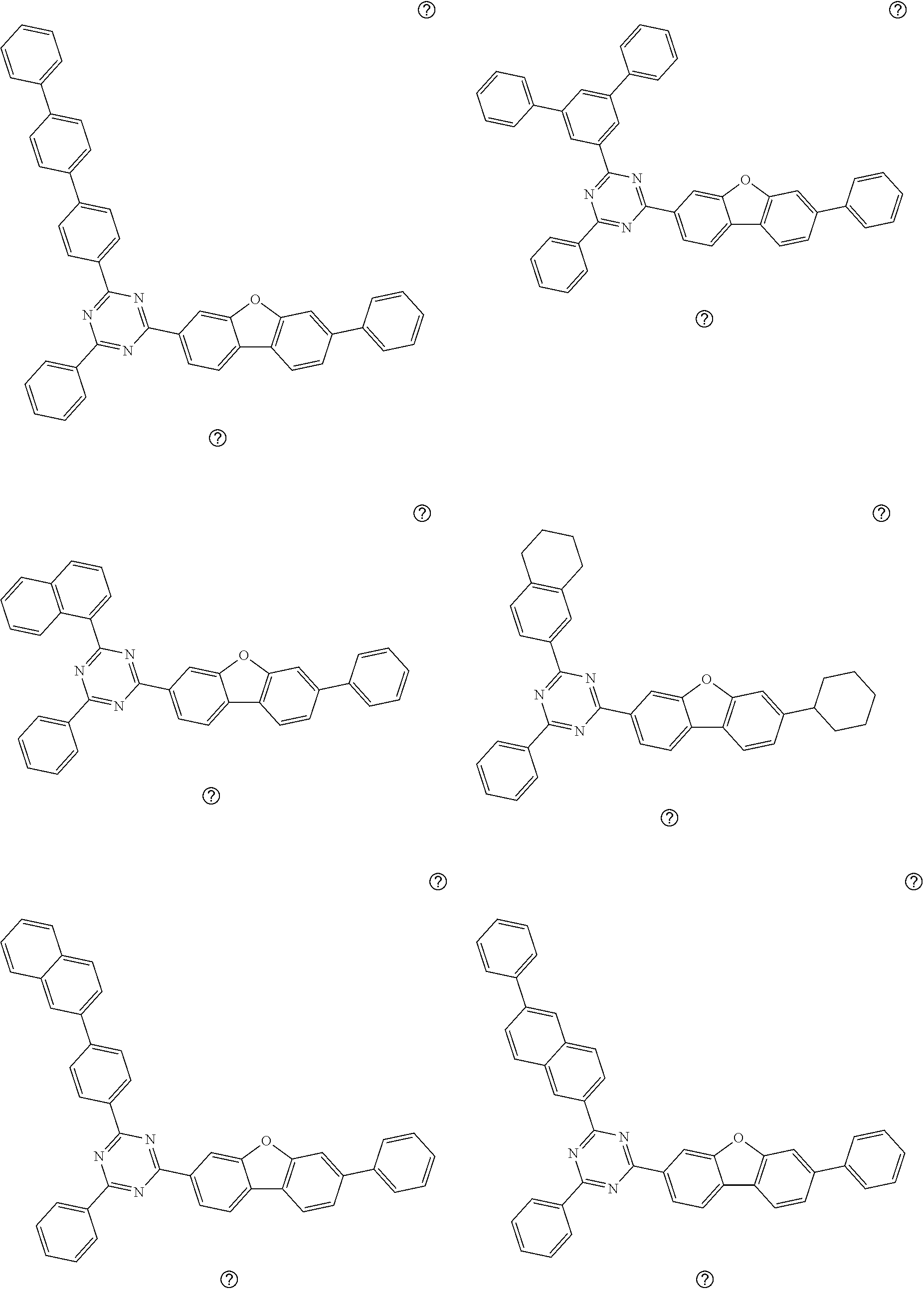 [SMILES: c1ccc(-c2ccc(-c3ccc(-c4nc(-c5ccccc5)nc(-c5ccc6c(c5)oc5cc(-c7ccccc7)ccc56)n4)cc3)cc2)cc1 ; c1ccc(-c2cc(-c3ccccc3)cc(-c3nc(-c4ccccc4)nc(-c4ccc5c(c4)oc4cc(-c6ccccc6)ccc45)n3)c2)cc1 ; c1ccc(-c2ccc3c(c2)oc2cc(-c4nc(-c5ccccc5)nc(-c5cccc6ccccc56)n4)ccc23)cc1 ; c1ccc(-c2nc(-c3ccc4c(c3)CCCC4)nc(-c3ccc4c(c3)oc3cc(C5CCCCC5)ccc34)n2)cc1 ; c1ccc(-c2ccc3c(c2)oc2cc(-c4nc(-c5ccccc5)nc(-c5ccc(-c6ccc7ccccc7c6)cc5)n4)ccc23)cc1 ; c1ccc(-c2ccc3cc(-c4nc(-c5ccccc5)nc(-c5ccc6c(c5)oc5cc(-c7ccccc7)ccc56)n4)ccc3c2)cc1]

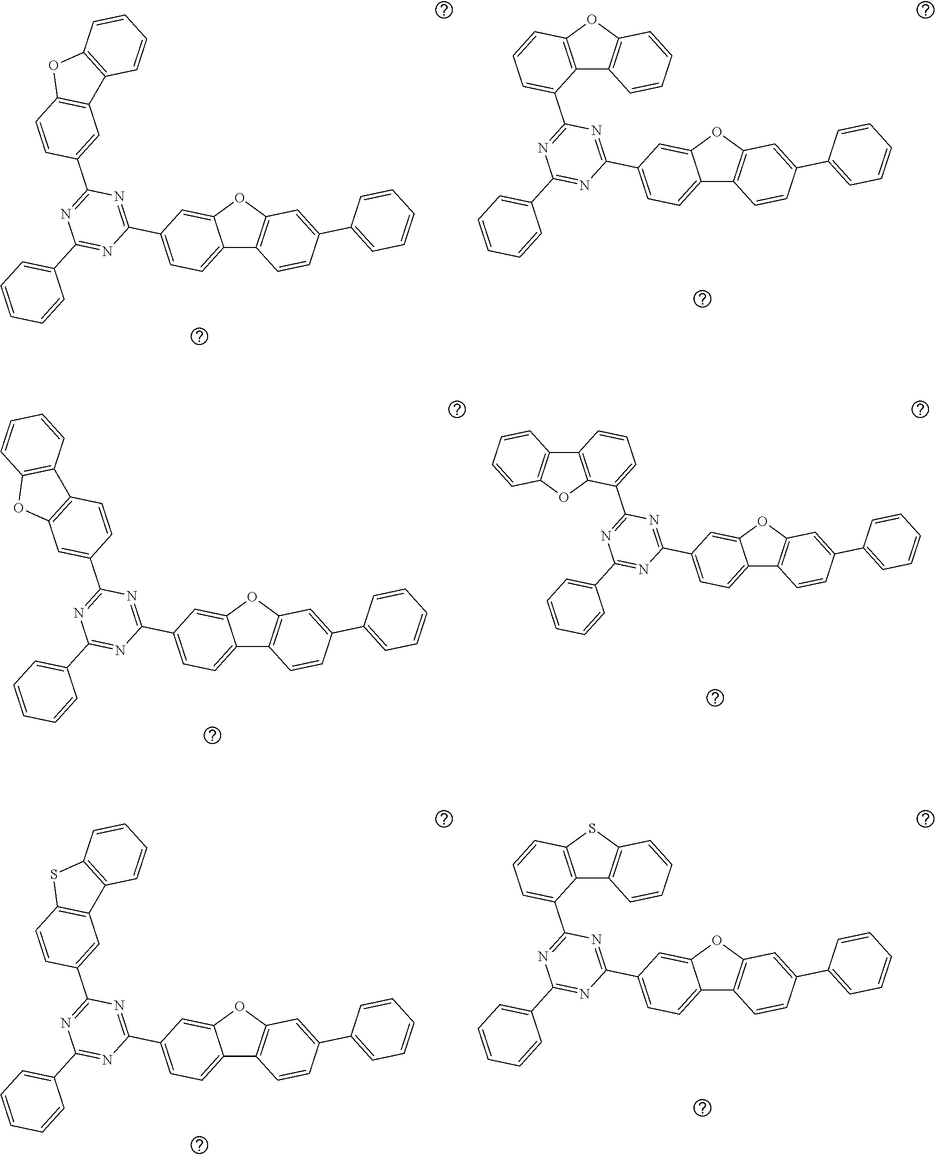 [SMILES: c1ccc(-c2ccc3c(c2)oc2cc(-c4nc(-c5ccccc5)nc(-c5ccc6oc7ccccc7c6c5)n4)ccc23)cc1 ; c1ccc(-c2ccc3c(c2)oc2cc(-c4nc(-c5ccccc5)nc(-c5cccc6oc7ccccc7c56)n4)ccc23)cc1 ; c1ccc(-c2ccc3c(c2)oc2cc(-c4nc(-c5ccccc5)nc(-c5ccc6c(c5)oc5ccccc56)n4)ccc23)cc1 ; c1ccc(-c2ccc3c(c2)oc2cc(-c4nc(-c5ccccc5)nc(-c5cccc6c5oc5ccccc56)n4)ccc23)cc1 ; c1ccc(-c2ccc3c(c2)oc2cc(-c4nc(-c5ccccc5)nc(-c5ccc6sc7ccccc7c6c5)n4)ccc23)cc1 ; c1ccc(-c2ccc3c(c2)oc2cc(-c4nc(-c5ccccc5)nc(-c5cccc6sc7ccccc7c56)n4)ccc23)cc1]

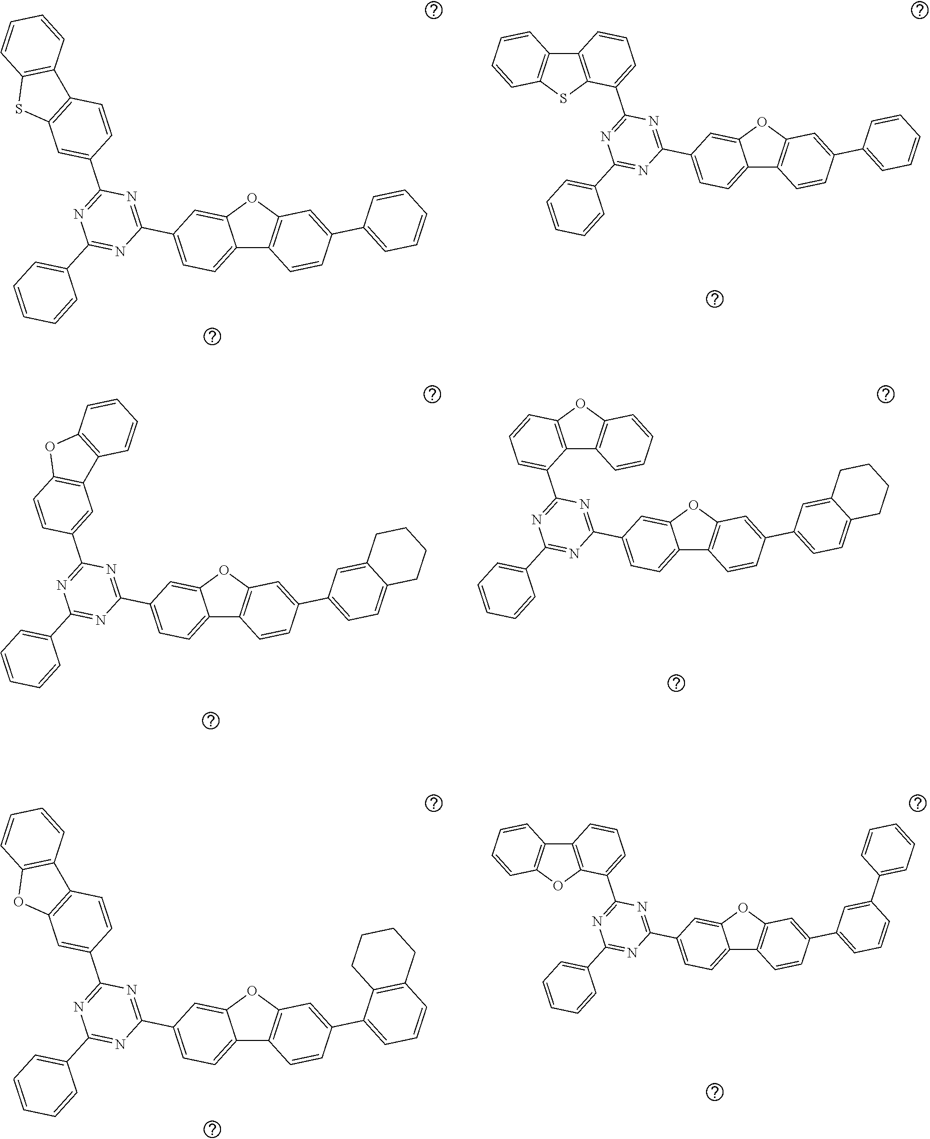

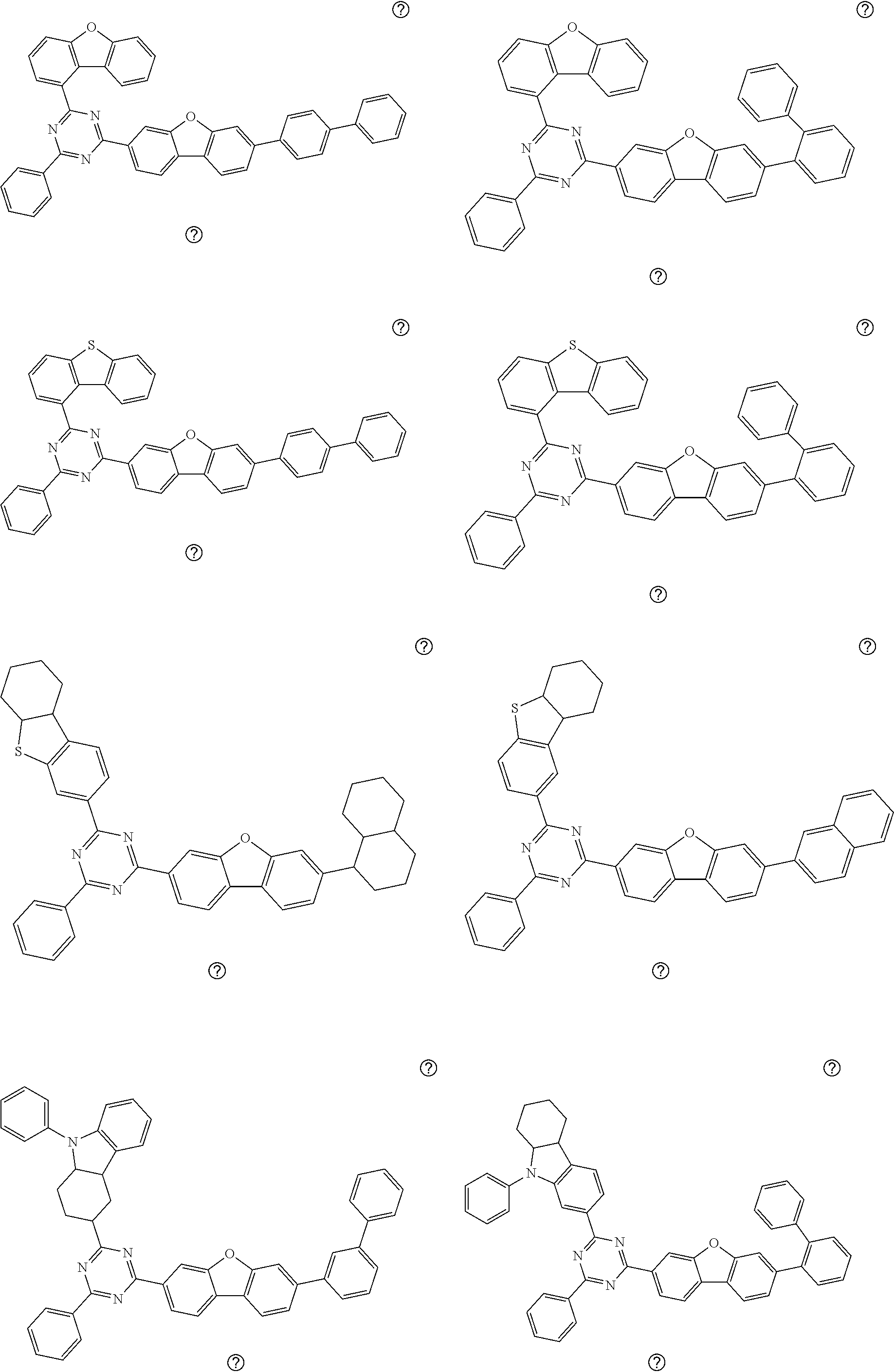

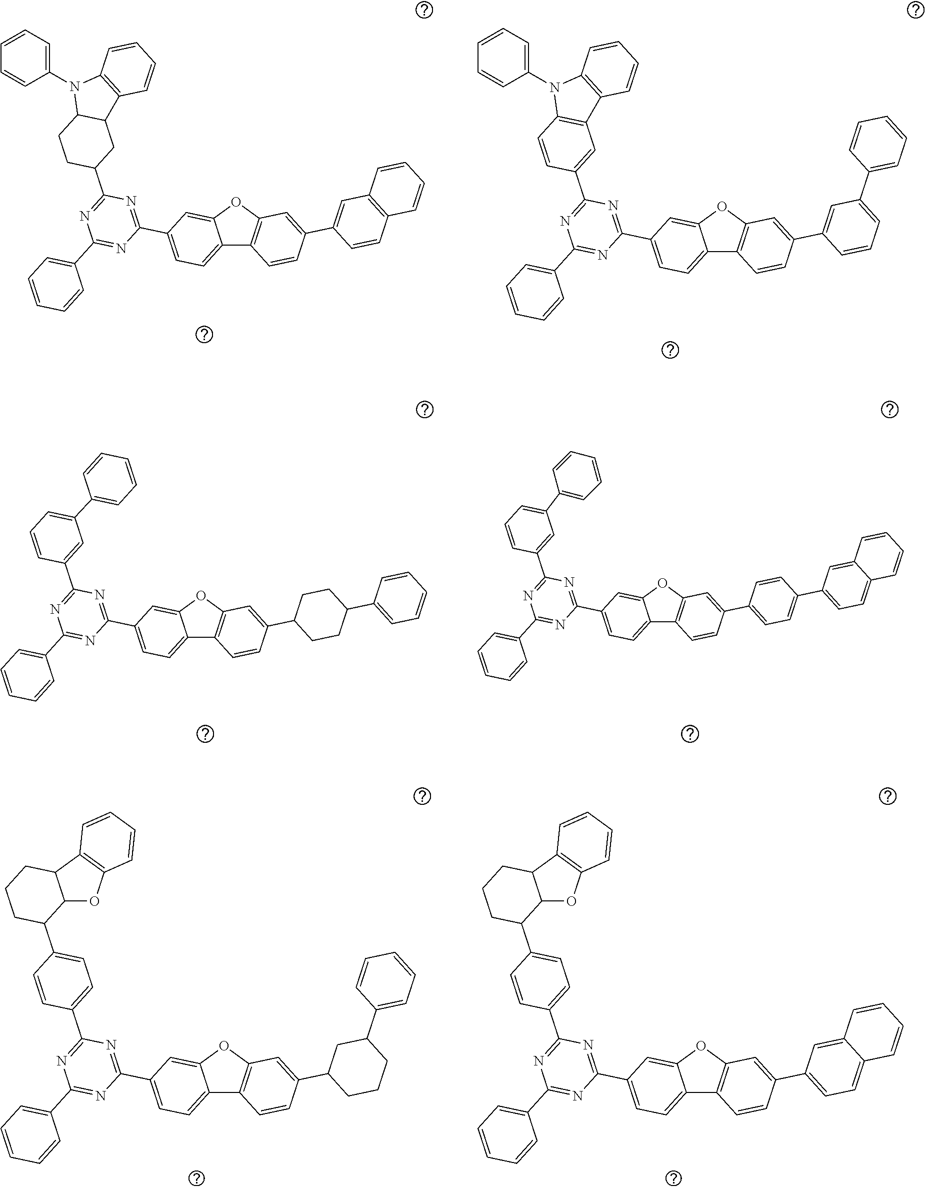

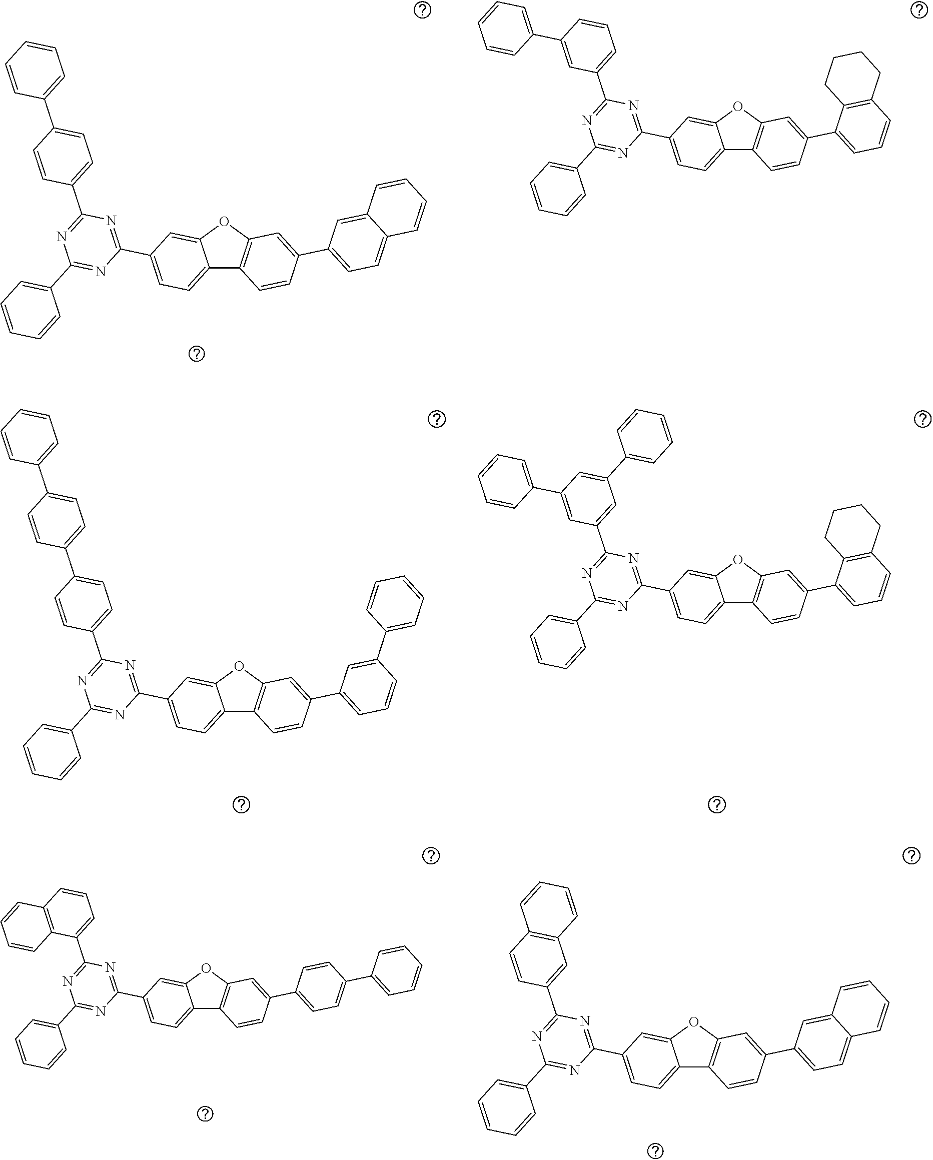

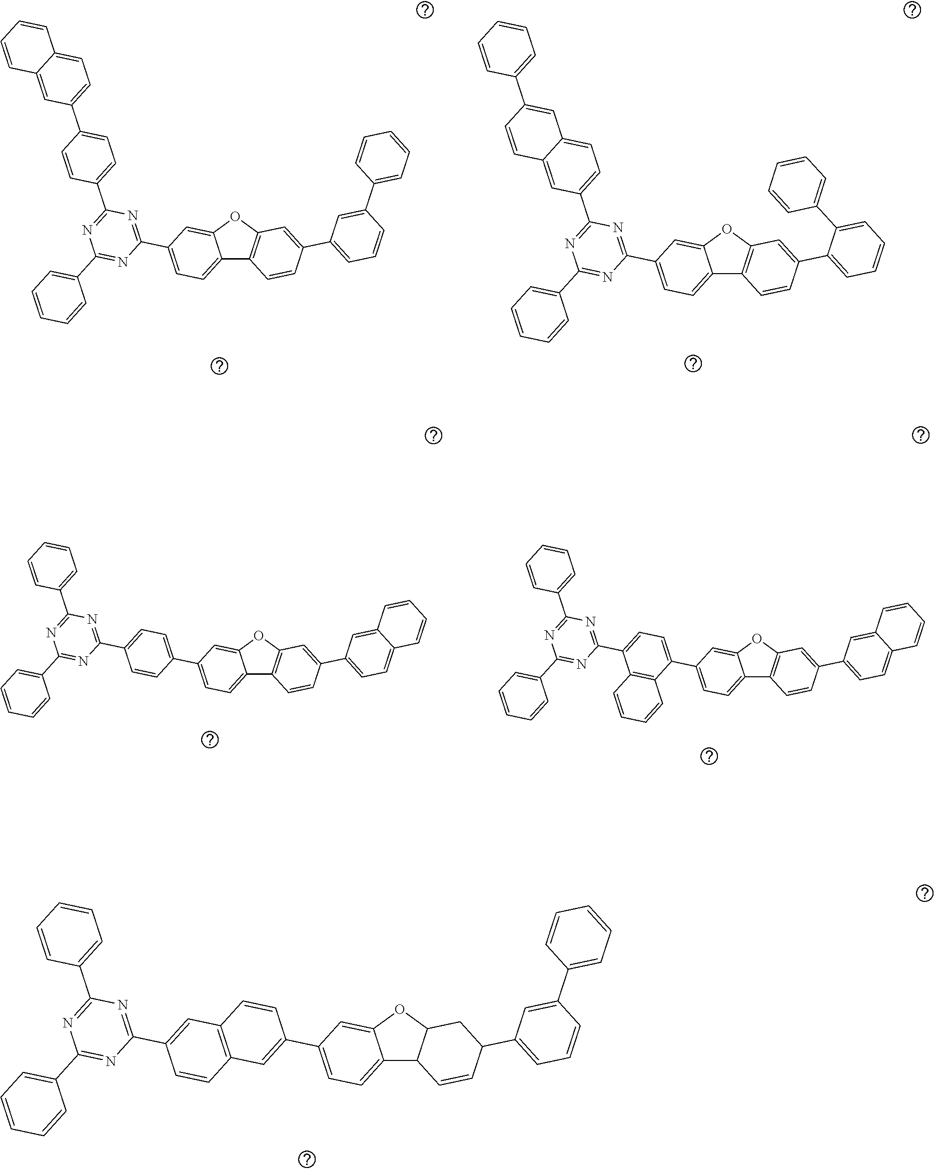 [SMILES: c1ccc(-c2cccc(-c3ccc4c(c3)oc3cc(-c5nc(-c6ccccc6)nc(-c6ccc(-c7ccc8ccccc8c7)cc6)n5)ccc34)c2)cc1 ; c1ccc(-c2ccc3cc(-c4nc(-c5ccccc5)nc(-c5ccc6c(c5)oc5cc(-c7ccccc7-c7ccccc7)ccc56)n4)ccc3c2)cc1 ; c1ccc(-c2nc(-c3ccccc3)nc(-c3ccc(-c4ccc5c(c4)oc4cc(-c6ccc7ccccc7c6)ccc45)cc3)n2)cc1 ; c1ccc(-c2nc(-c3ccccc3)nc(-c3ccc(-c4ccc5c(c4)oc4cc(-c6ccc7ccccc7c6)ccc45)c4ccccc34)n2)cc1 ; C1=CC2c3ccc(-c4ccc5cc(-c6nc(-c7ccccc7)nc(-c7ccccc7)n6)ccc5c4)cc3OC2CC1c1cccc(-c2ccccc2)c1]

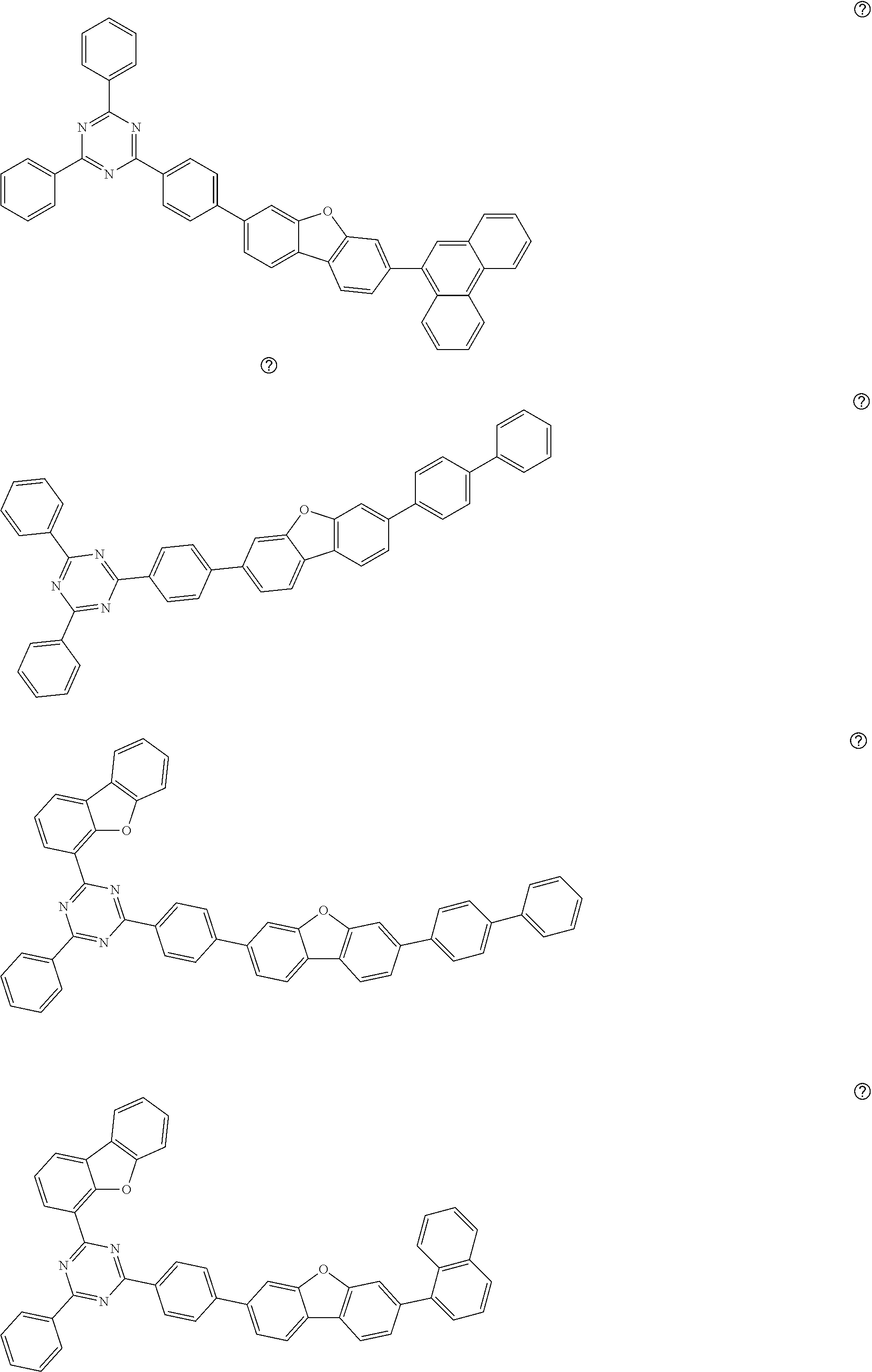 [SMILES: c1ccc(-c2nc(-c3ccccc3)nc(-c3ccc(-c4ccc5c(c4)oc4cc(-c6cc7ccccc7c7ccccc67)ccc45)cc3)n2)cc1 ; c1ccc(-c2ccc(-c3ccc4c(c3)oc3cc(-c5ccc(-c6nc(-c7ccccc7)nc(-c7ccccc7)n6)cc5)ccc34)cc2)cc1 ; c1ccc(-c2ccc(-c3ccc4c(c3)oc3cc(-c5ccc(-c6nc(-c7ccccc7)nc(-c7cccc8c7oc7ccccc78)n6)cc5)ccc34)cc2)cc1 ; c1ccc(-c2nc(-c3ccc(-c4ccc5c(c4)oc4cc(-c6cccc7ccccc67)ccc45)cc3)nc(-c3cccc4c3oc3ccccc34)n2)cc1]

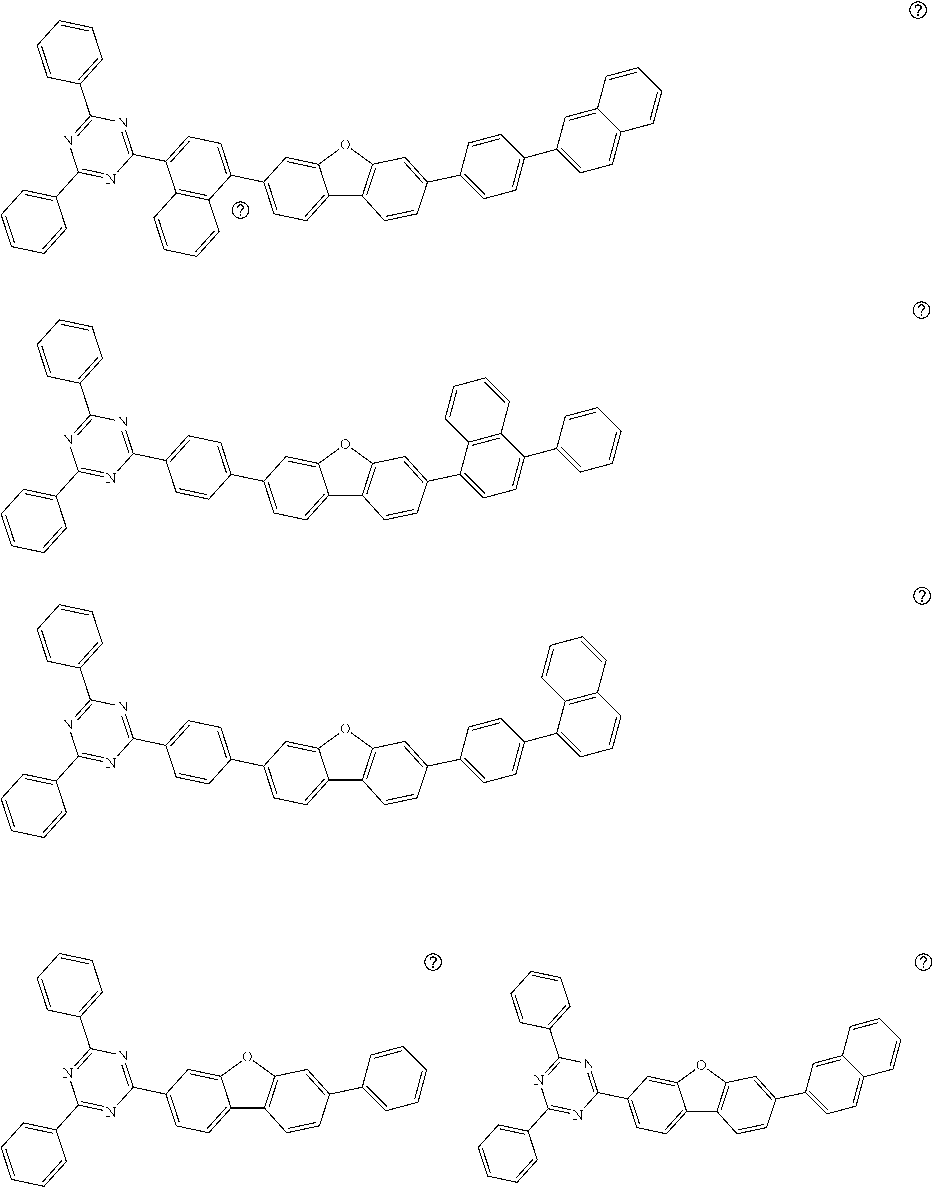 [SMILES: c1ccc(-c2nc(-c3ccccc3)nc(-c3ccc(-c4ccc5c(c4)oc4cc(-c6ccc(-c7ccc8ccccc8c7)cc6)ccc45)c4ccccc34)n2)cc1 ; c1ccc(-c2nc(-c3ccccc3)nc(-c3ccc(-c4ccc5c(c4)oc4cc(-c6ccc(-c7ccccc7)c7ccccc67)ccc45)cc3)n2)cc1 ; c1ccc(-c2nc(-c3ccccc3)nc(-c3ccc(-c4ccc5c(c4)oc4cc(-c6ccc(-c7cccc8ccccc78)cc6)ccc45)cc3)n2)cc1 ; c1ccc(-c2ccc3c(c2)oc2cc(-c4nc(-c5ccccc5)nc(-c5ccccc5)n4)ccc23)cc1 ; c1ccc(-c2nc(-c3ccccc3)nc(-c3ccc4c(c3)oc3cc(-c5ccc6ccccc6c5)ccc34)n2)cc1]

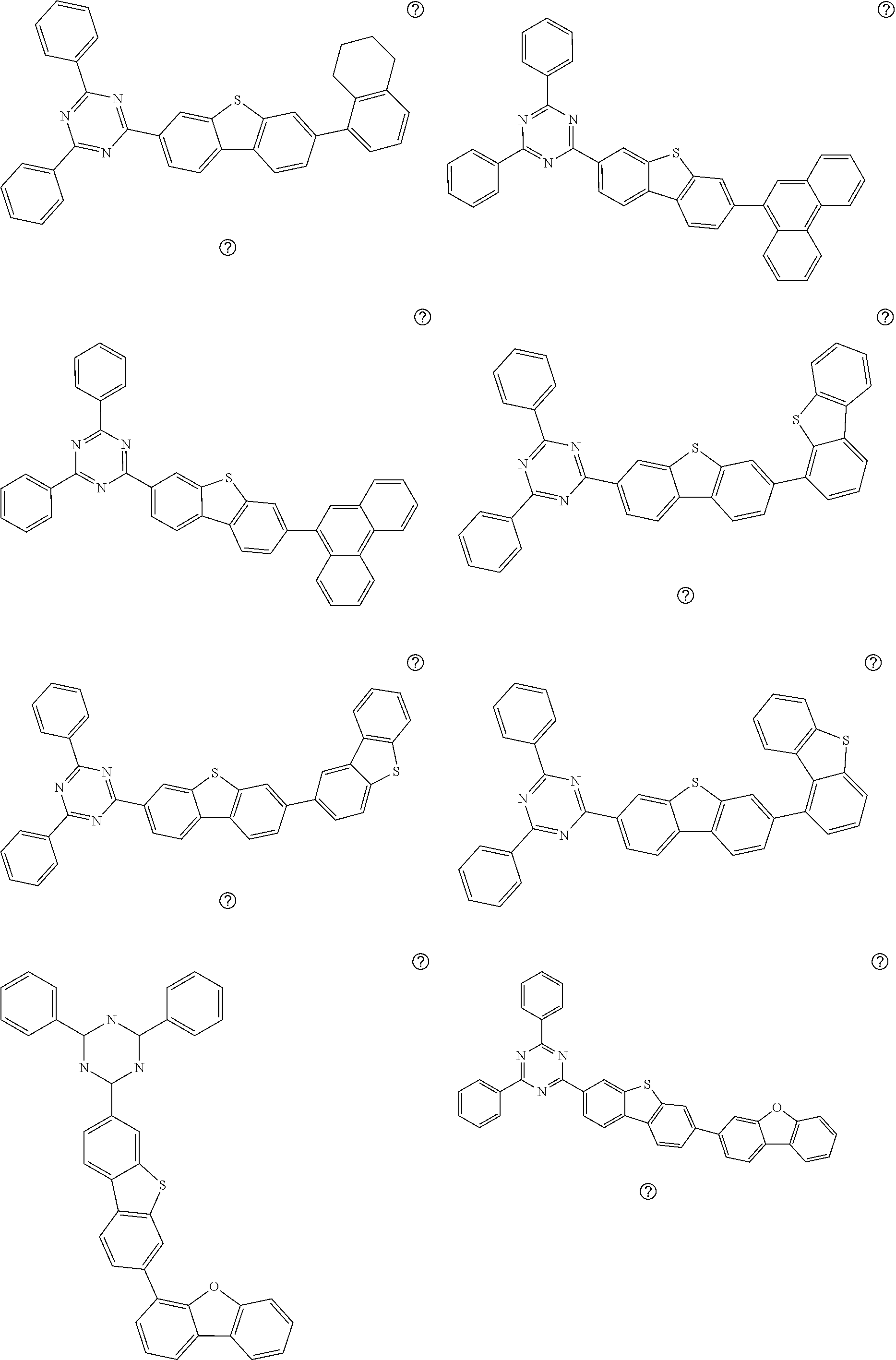

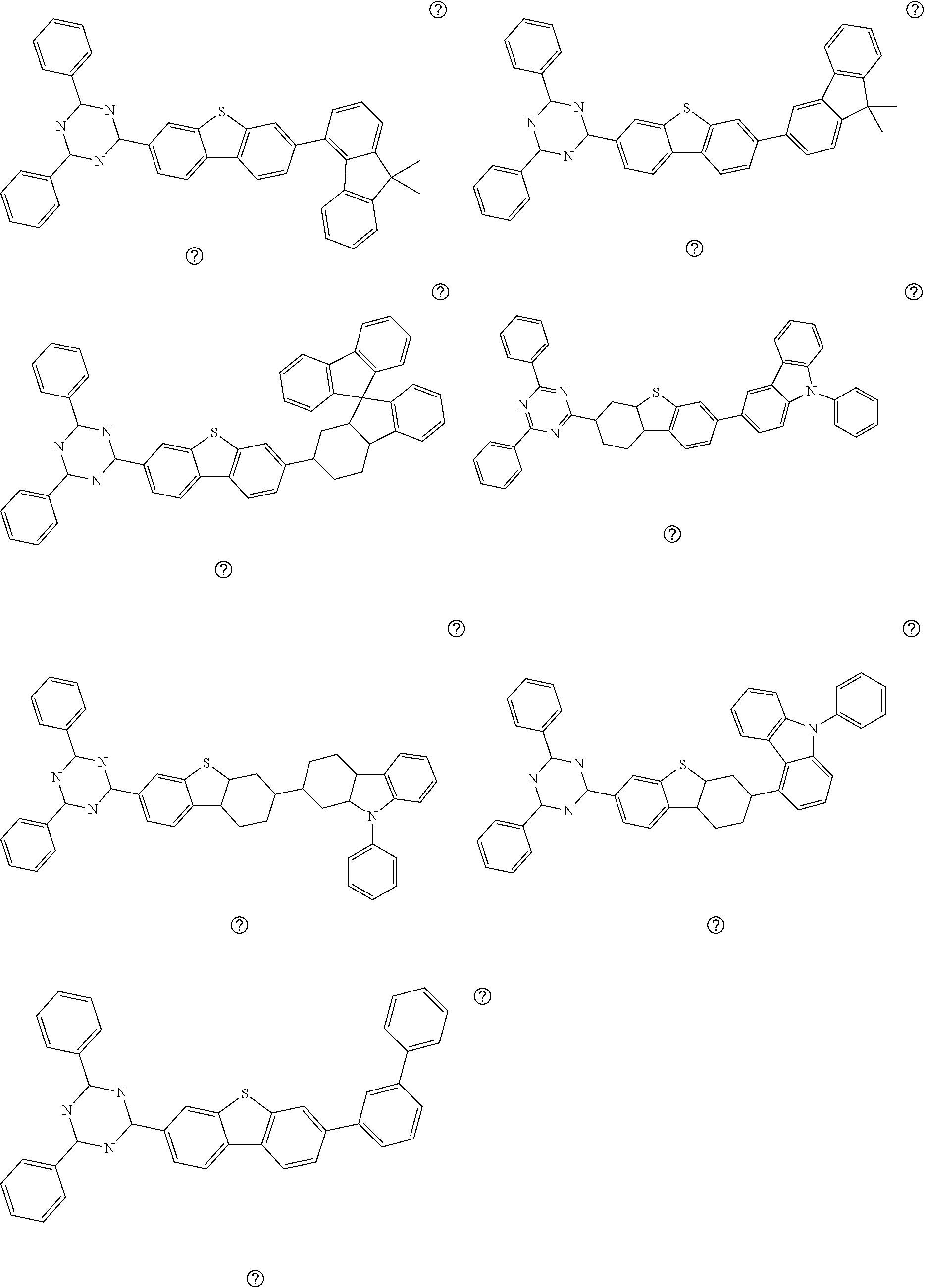

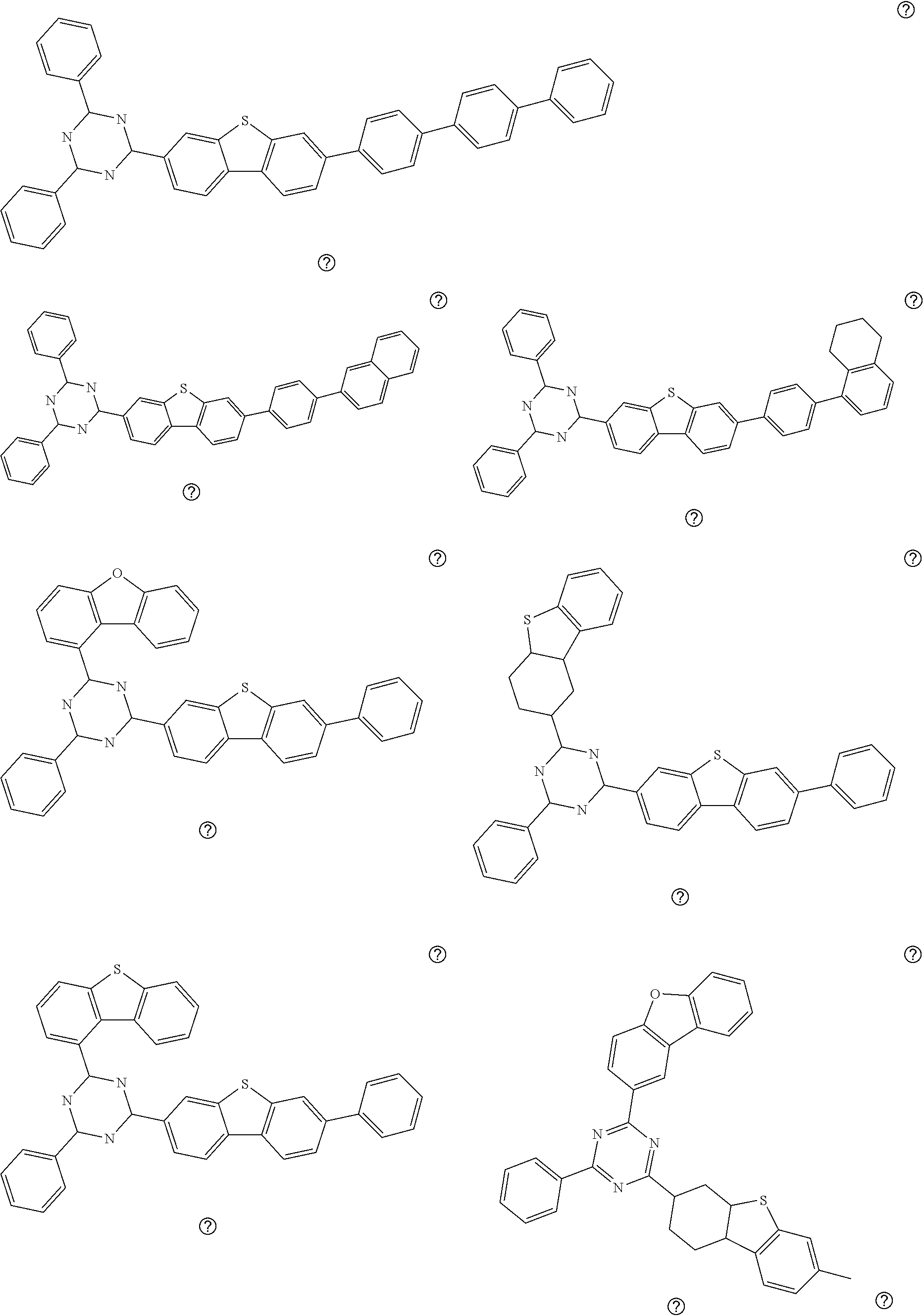

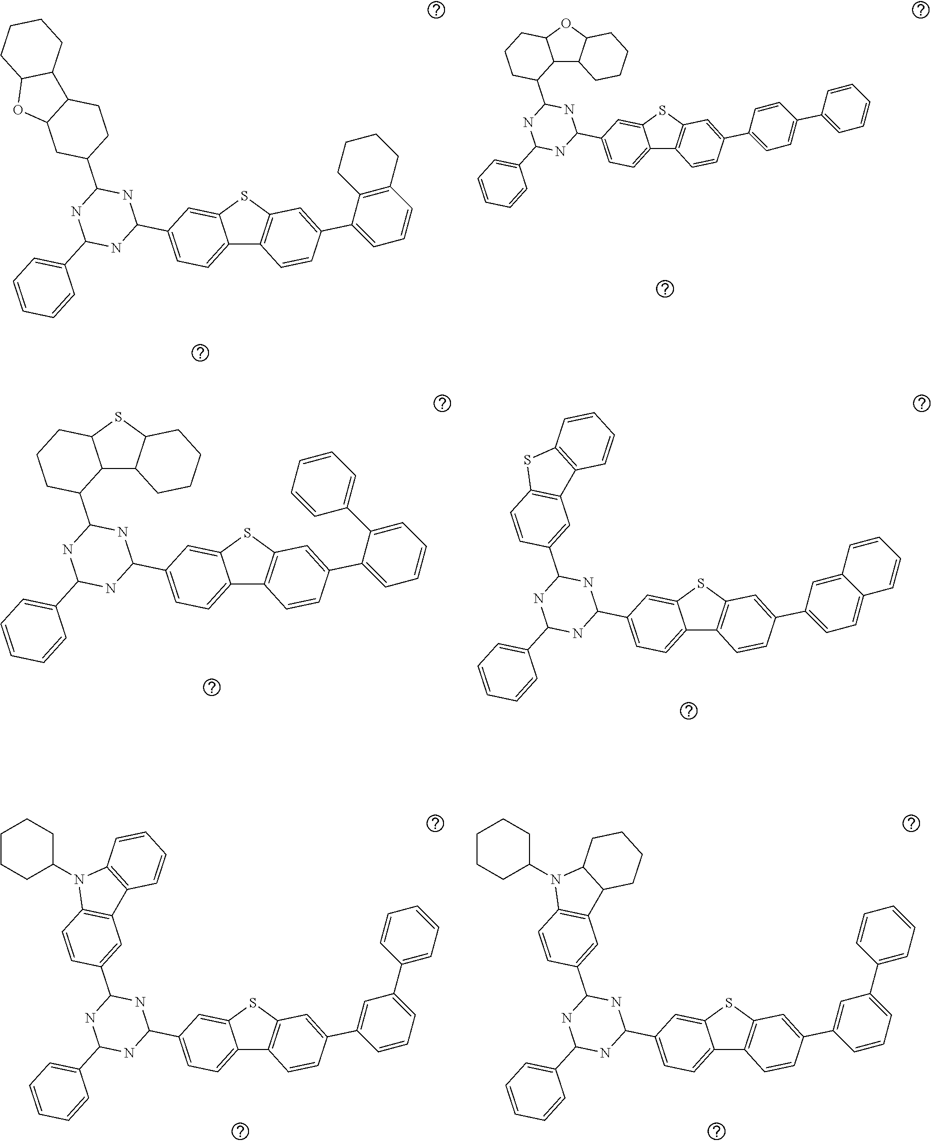

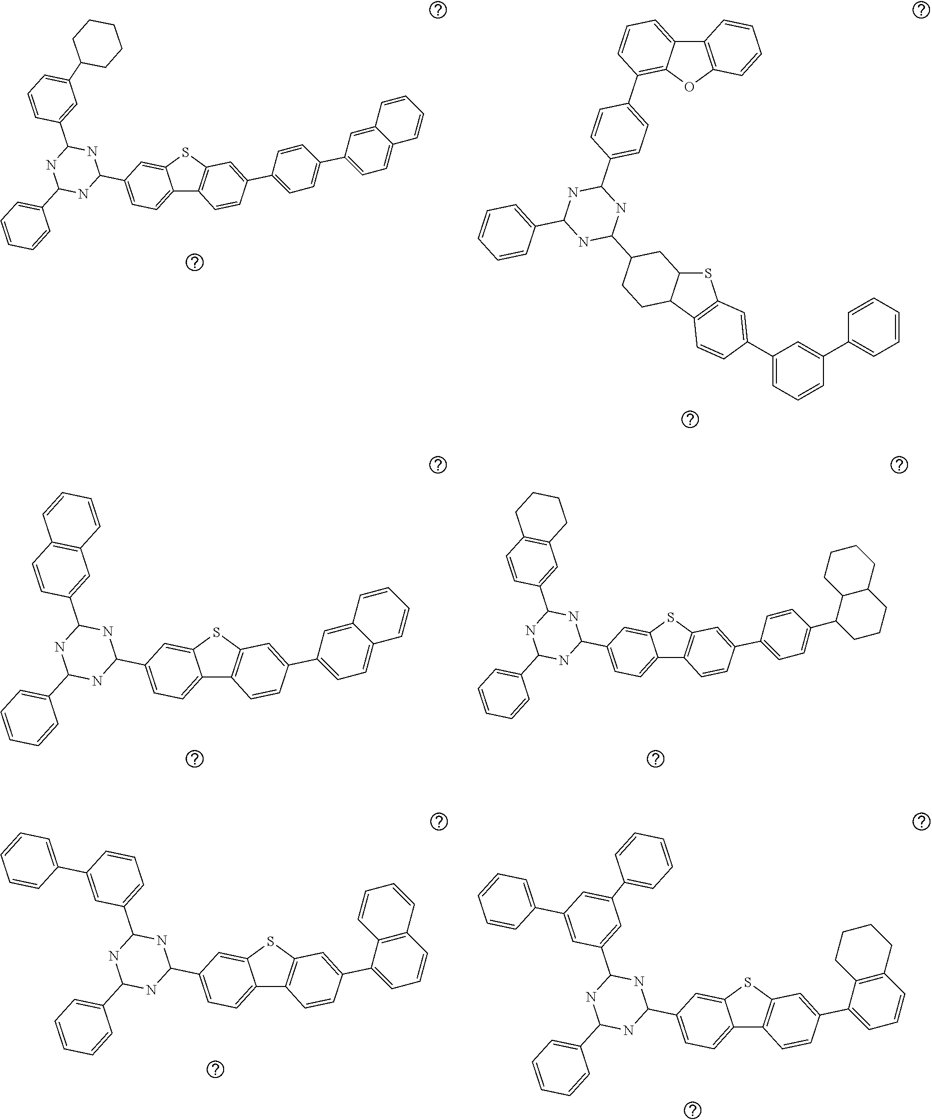

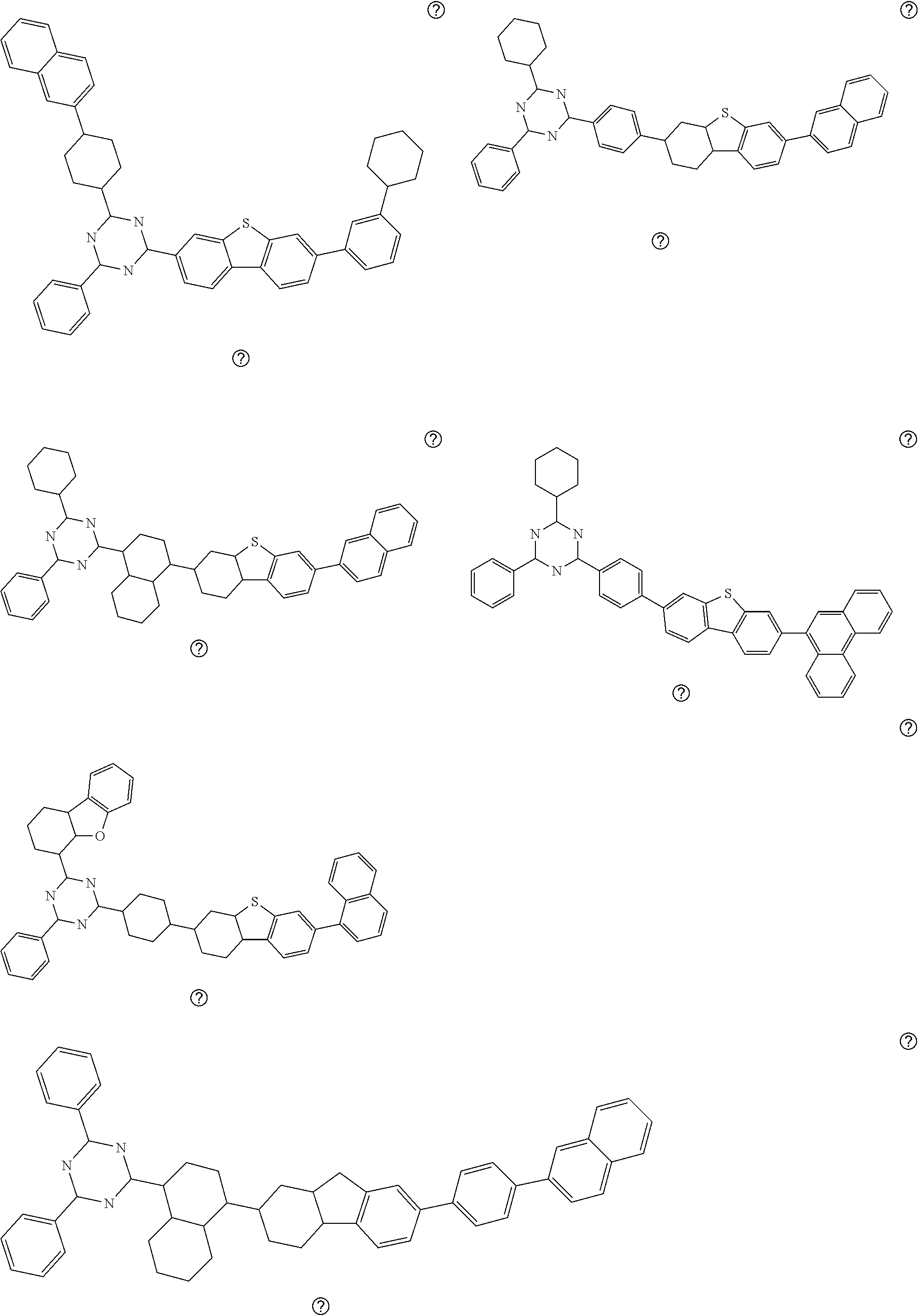

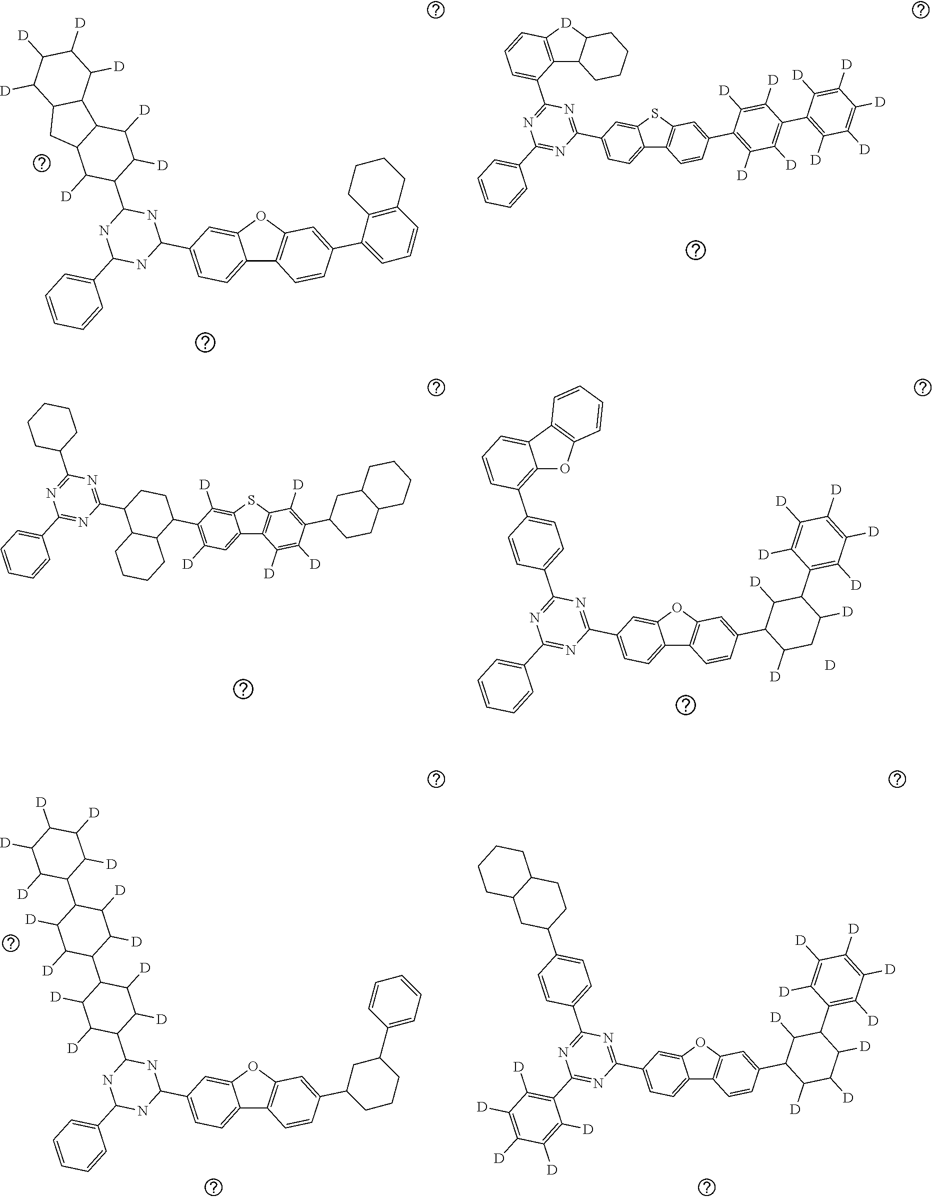

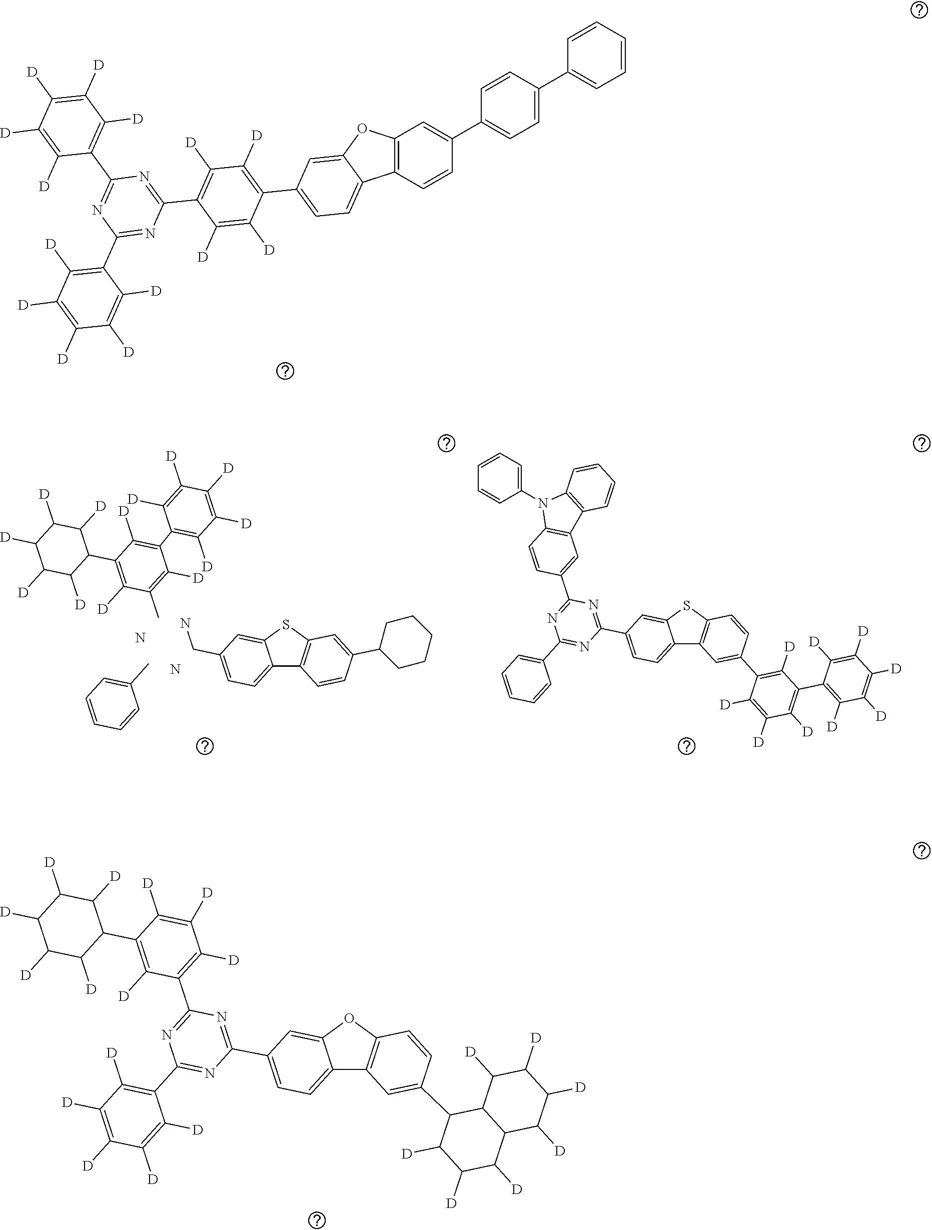

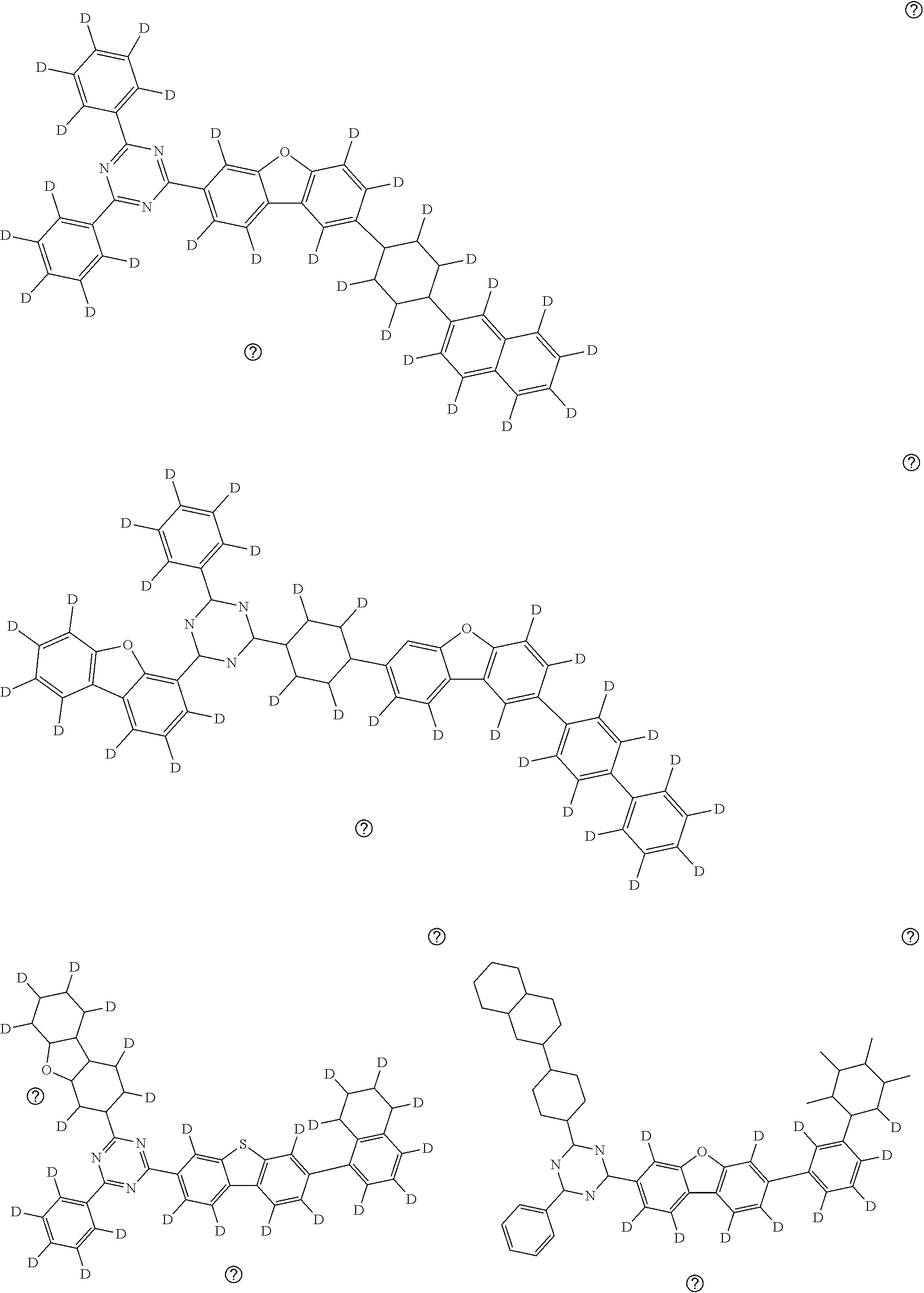

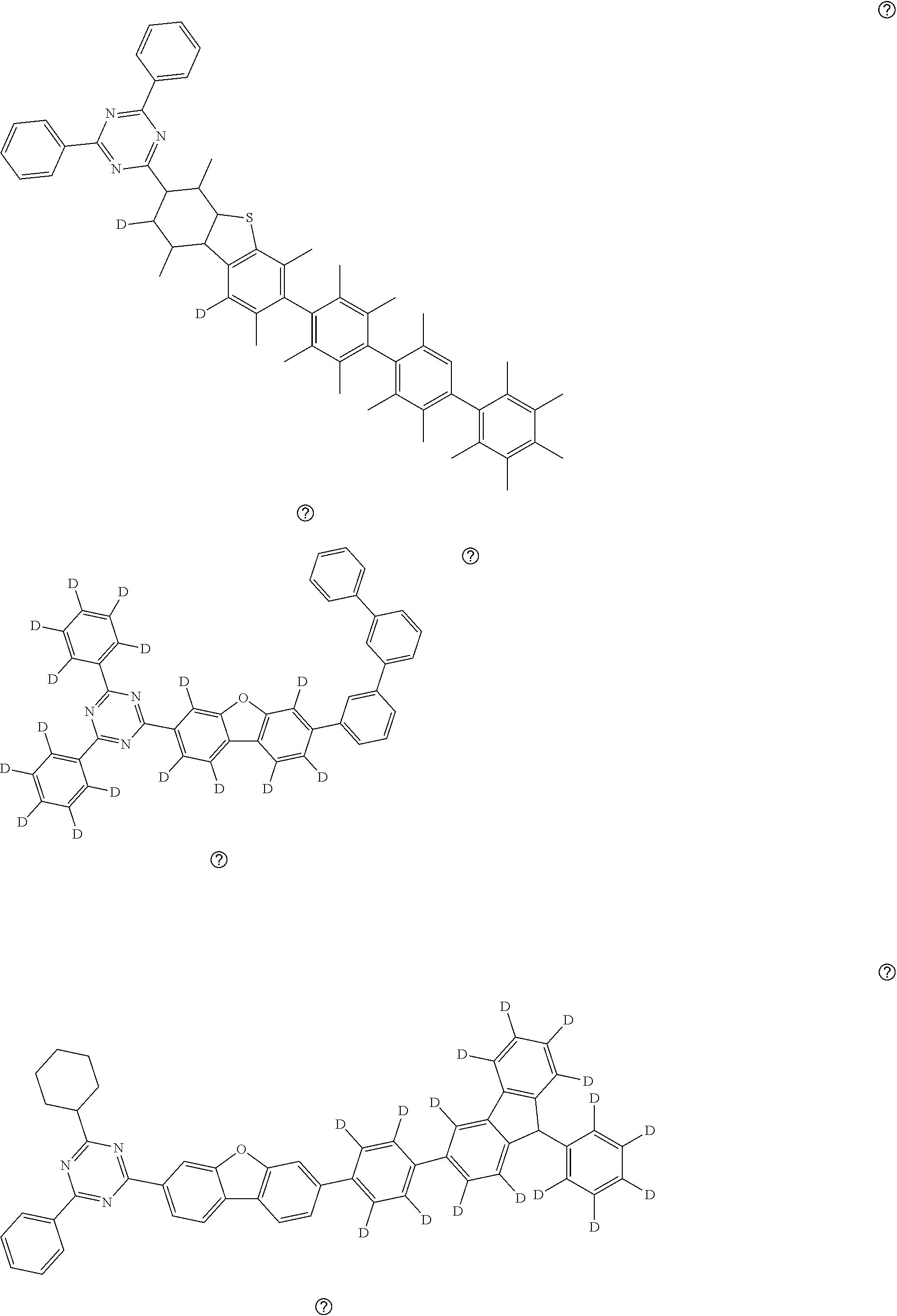

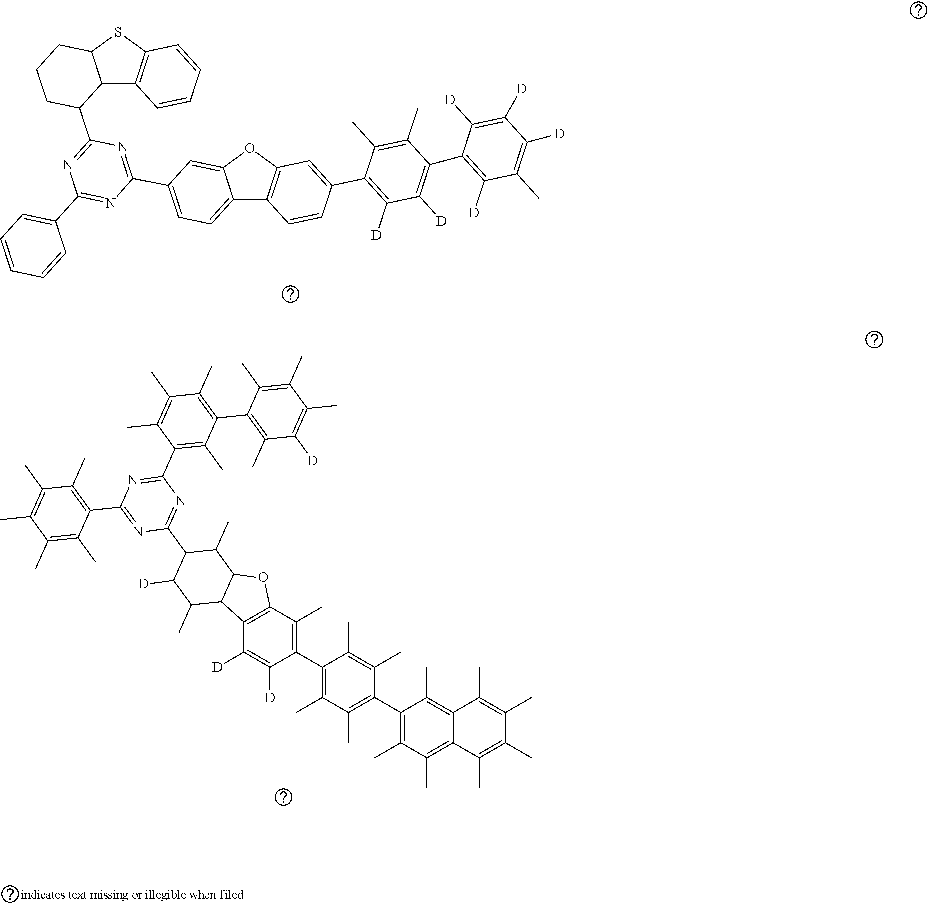

In an exemplary embodiment of the present application, the N-Het2 may be represented by any one of the following Chemical Formulae 2-1 to 2-3.

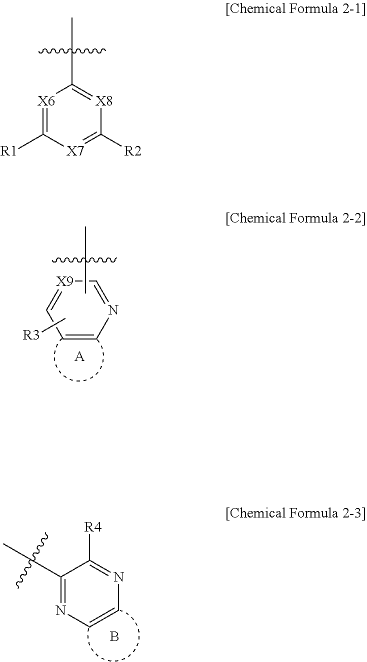

In Chemical Formulae 2-1 to 2-3,

X6 to X9 are each N or CR16,

at least one of X6 to X8 is N,

A and B are the same as or different from each other, and are each independently a substituted or unsubstituted monocyclic or polycyclic C6 to C60 aryl ring; or a substituted or unsubstituted monocyclic or polycyclic C2 to C60 hetero ring, and

R1 to R4 and R16 are the same as or different from each other, and are each independently hydrogen; deuterium; a halogen group; a cyano group; a substituted or unsubstituted C1 to C60 alkyl group; a substituted or unsubstituted C6 to C60 aryl group; or a substituted or unsubstituted C2 to C60 heteroaryl group.

In an exemplary embodiment of the present application, Chemical Formula 2-1 may be selected from the following structural formulae.

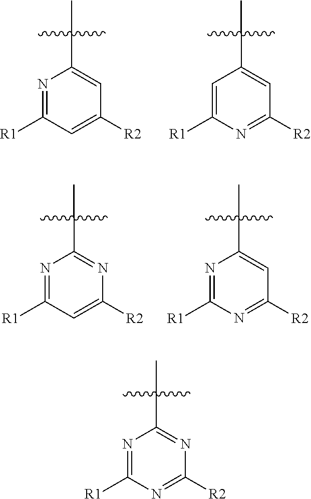

The definitions of R1 and R2 are the same as the definitions in Chemical Formula 2-1.

In an exemplary embodiment of the present application, Chemical Formula 2-2 may be represented by any one of the following Chemical Formulae 2-2-1 to 2-2-3.

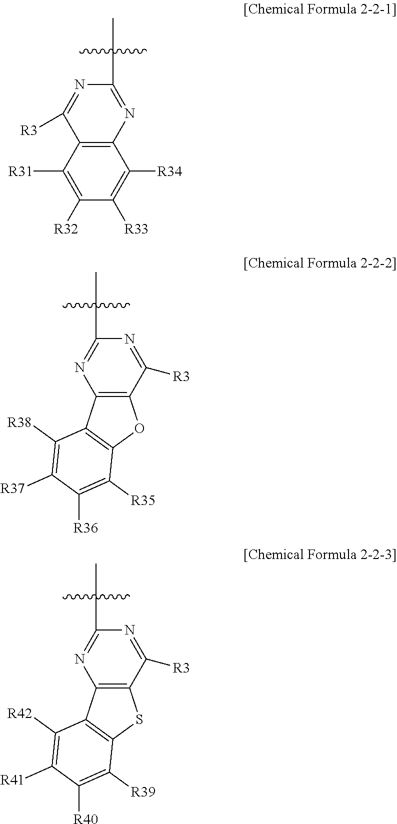

In Chemical Formula 2-2-1, R3 is the same as the definition in Chemical Formula 2-2, and

R31 to R42 are the same as or different from each other, and are each independently hydrogen; or deuterium.

In an exemplary embodiment of the present application, Chemical Formula 2-3 may be represented by the following Chemical Formula 2-3-1.

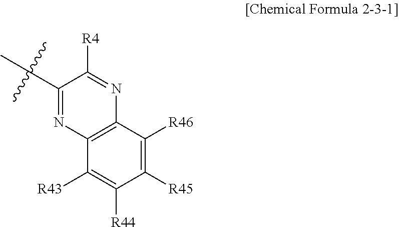

In Chemical Formula 2-3-1, R4 is the same as the definition in Chemical Formula 2-3, and R43 to R46 are the same as or different from each other, and are each independently hydrogen; or deuterium.

In an exemplary embodiment of the present application, Ar2 and Ar3 are the same as or different from each other, and may be each independently a substituted or unsubstituted C6 to C40 aryl group; or a substituted or unsubstituted C2 to C40 heteroaryl group.

In another exemplary embodiment, Ar2 and Ar3 are the same as or different from each other, and may be each independently a substituted or unsubstituted C6 to C20 aryl group; or a substituted or unsubstituted C2 to C20 heteroaryl group.

In still another exemplary embodiment, Ar2 and Ar3 are the same as or different from each other, and may be each independently a substituted or unsubstituted phenyl group; a substituted or unsubstituted biphenyl group; a substituted or unsubstituted terphenyl group; a substituted or unsubstituted naphthyl group; a substituted or unsubstituted phenanthrene group; a substituted or unsubstituted fluorenyl group; a substituted or unsubstituted dibenzofuran group; a substituted or unsubstituted dibenzothiophene group; a substituted or unsubstituted carbazole group; a substituted or unsubstituted naphthobenzofuran group; or a substituted or unsubstituted naphthobenzothiophene group.

In yet another exemplary embodiment, Ar2 and Ar3 are the same as or different from each other, and may be each independently a phenyl group which is unsubstituted or substituted with deuterium, a cyano group, a halogen group, an amine group substituted with a naphthyl group or a phenyl group or a dibenzofuran group; a biphenyl group which is unsubstituted or substituted with deuterium; a terphenyl group which is unsubstituted or substituted with deuterium; a naphthyl group which is unsubstituted or substituted with deuterium or a phenyl group; a phenanthrene group; a fluorenyl group which is unsubstituted or substituted with a methyl group; a spirobifluorenyl group; a dibenzofuran group which is unsubstituted or substituted with deuterium or a phenyl group; a dibenzothiophene group which is unsubstituted or substituted with deuterium or a phenyl group; a naphthobenzofuran group; or a naphthobenzothiophene group.

In an exemplary embodiment of the present application, the N-Het2 may be a C2 to C40 heteroaryl group which is substituted or unsubstituted and includes N.

In another exemplary embodiment, the N-Het2 may be a C2 to C20 heteroaryl group which is substituted or unsubstituted and includes N.

In still another exemplary embodiment, the N-Het2 may be a substituted or unsubstituted pyridine group; a substituted or unsubstituted pyrimidine group; a substituted or unsubstituted triazine group; a substituted or unsubstituted quinazoline group; a substituted or unsubstituted quinoxaline group; a substituted or unsubstituted benzofuropyrimidine group; or a substituted or unsubstituted benzothienopyrimidine group.

In yet another exemplary embodiment, the N-Het2 may be a substituted or unsubstituted pyrimidine group; a substituted or unsubstituted triazine group; a substituted or unsubstituted quinazoline group; a substituted or unsubstituted quinoxaline group; a substituted or unsubstituted benzofuropyrimidine group; or a substituted or unsubstituted benzothienopyrimidine group.

In still yet another exemplary embodiment, the N-Het2 may be a pyrimidine group substituted with a phenyl group or a dibenzofuran group; a triazine group substituted with a phenyl group unsubstituted or substituted with a halogen group or a naphthyl group, a biphenyl group, a naphthyl group unsubstituted or substituted with a phenyl group, a terphenyl group, a dibenzofuran group, a dibenzothiophene group, or a carbazole group unsubstituted or substituted with a phenyl group; a quinazoline group substituted with a phenyl group, a biphenyl group or a naphthyl group; a quinoxaline group substituted with a phenyl group, a biphenyl group or a naphthyl group; a benzofuropyrimidine group substituted with a phenyl group, a biphenyl group or a naphthyl group; or a benzothienopyrimidine group substituted with a phenyl group, a biphenyl group or a naphthyl group.

In an exemplary embodiment of the present application, t2, r2 and r3 are each an integer from 1 to 2, and when t2 is 2, N-Het2's are the same as or different from each other, and when r2 is 2, Ar2's are the same as or different from each other, and when r3 is 2, Ar3's may be the same as or different from each other.

In another exemplary embodiment, t2, r2 and r3 are 1.

In still another exemplary embodiment, L3 and L4 are the same as or different from each other, and may be each independently a direct bond; a substituted or unsubstituted C6 to C40 arylene group; or a substituted or unsubstituted C2 to C40 heteroarylene group.

In yet another exemplary embodiment, L3 and L4 are the same as or different from each other, and may be each independently a direct bond; or a substituted or unsubstituted C6 to C20 arylene group.

In still yet another further exemplary embodiment, L3 and L4 are the same as or different from each other, and may be each independently a direct bond; a substituted or unsubstituted phenylene group; a substituted or unsubstituted biphenylene group; or a substituted or unsubstituted naphthylene group.

In a further exemplary embodiment, L3 and L4 are the same as or different from each other, and may be each independently a direct bond; a phenylene group; a biphenylene group; or a naphthylene group.

In an exemplary embodiment of the present application, r and s are each an integer from 0 to 2, and when r is 2 or higher, L3's are the same as or different from each other, and when s is 2 or higher, L4's may be the same as or different from each other.

In an exemplary embodiment of the present application, Rc and Rd are the same as or different from each other, and may be each independently hydrogen; deuterium; a halogen group; a cyano group; a substituted or unsubstituted C1 to C20 alkyl group; a substituted or unsubstituted C6 to C20 aryl group; or a substituted or unsubstituted C2 to C20 heteroaryl group.

In another exemplary embodiment, Rc and Rd are the same as or different from each other, and may be each independently hydrogen; deuterium; a substituted or unsubstituted phenyl group or a substituted or unsubstituted biphenyl group.

In still another exemplary embodiment, Rc and Rd are the same as or different from each other, and may be each independently hydrogen; or deuterium.

In an exemplary embodiment of the present application, c and d are each an integer from 0 to 2, and when c is 2 or higher, Rc's are the same as or different from each other, and when d is 2 or higher, Rd's may be the same as or different from each other.

In an exemplary embodiment of the present application, X6 to X8 may all be N.

In an exemplary embodiment of the present application, X9 may be N.

In an exemplary embodiment of the present application, A may be a substituted or unsubstituted benzene ring; a substituted or unsubstituted quinoline ring; a substituted or unsubstituted indole ring; a substituted or unsubstituted benzofuran ring; or a substituted or unsubstituted benzothiophene ring.

In another exemplary embodiment, A may be a benzene ring; a quinoline ring; an indole ring substituted with an aryl group; a benzofuran ring; or a benzothiophene ring. In an exemplary embodiment of the present application, B may be a substituted or unsubstituted monocyclic aryl ring.

In another exemplary embodiment, B may be a benzene ring.

In an exemplary embodiment of the present application, R1 to R4 and R16 are the same as or different from each other, and may be each independently hydrogen; deuterium; a halogen group; a cyano group; a substituted or unsubstituted C1 to C40 alkyl group; a substituted or unsubstituted C6 to C40 aryl group; or a substituted or unsubstituted C2 to C40 heteroaryl group.

In another exemplary embodiment, R1 to R4 and R16 are the same as or different from each other, and may be each independently hydrogen; deuterium; a halogen group; a cyano group; a substituted or unsubstituted C1 to C20 alkyl group; a substituted or unsubstituted C6 to C20 aryl group; or a substituted or unsubstituted C2 to C20 heteroaryl group.

In still another exemplary embodiment, R1 to R4 and R16 are the same as or different from each other, and may be each independently a substituted or unsubstituted phenyl group; a substituted or unsubstituted biphenyl group; a substituted or unsubstituted terphenyl group; a substituted or unsubstituted naphthyl group; a substituted or unsubstituted dibenzofuran group; a substituted or unsubstituted dibenzothiophene group; or a substituted or unsubstituted carbazole group.

In yet another exemplary embodiment, R1 to R4 and R16 are the same as or different from each other, and may be each independently a substituted or unsubstituted phenyl group; a substituted or unsubstituted biphenyl group; a substituted or unsubstituted terphenyl group; a substituted or unsubstituted naphthyl group; a substituted or unsubstituted dibenzofuran group; a substituted or unsubstituted dibenzothiophene group; or a substituted or unsubstituted carbazole group.

In an exemplary embodiment of the present application, the deuterium content of the compound of Chemical Formula 2 may be 0%, or 1% to 100%.

In an exemplary embodiment of the present application, the deuterium content of the compound of Chemical Formula 2 may be 0%, or 10% to 100%.

In an exemplary embodiment of the present application, the deuterium content of the compound of Chemical Formula 2 may be 0%, or 15% to 100%.

In an exemplary embodiment of the present application, the deuterium content of the compound of Chemical Formula 2 may be 0%, or 30% to 100%.

In an exemplary embodiment of the present application, the deuterium content of the compound of Chemical Formula 2 may be 0%, or 50% to 100%.

In an exemplary embodiment of the present application, the deuterium content of the compound of Chemical Formula 2 may be 0%, or 70% to 100%.

In an exemplary embodiment of the present application, the deuterium content of the compound of Chemical Formula 2 may be 0%, or 90% to 100%.

In an exemplary embodiment of the present application, the deuterium content of the compound of Chemical Formula 2 may be 0%.

In an exemplary embodiment of the present application, the deuterium content of the compound of Chemical Formula 2 may be 15% to 100%.

In an exemplary embodiment of the present application, the deuterium content of the compound of Chemical Formula 2 may be 30% to 100%.

In an exemplary embodiment of the present application, the deuterium content of the compound of Chemical Formula 2 may be 50% to 100%.

In an exemplary embodiment of the present application, the deuterium content of the compound of Chemical Formula 2 may be 70% to 100%.

In an exemplary embodiment of the present application, the deuterium content of the compound of Chemical Formula 2 may be 90% to 100%.

In an exemplary embodiment of the present application, provided is a compound in which Chemical Formula 2 is represented by any one of the following compounds.

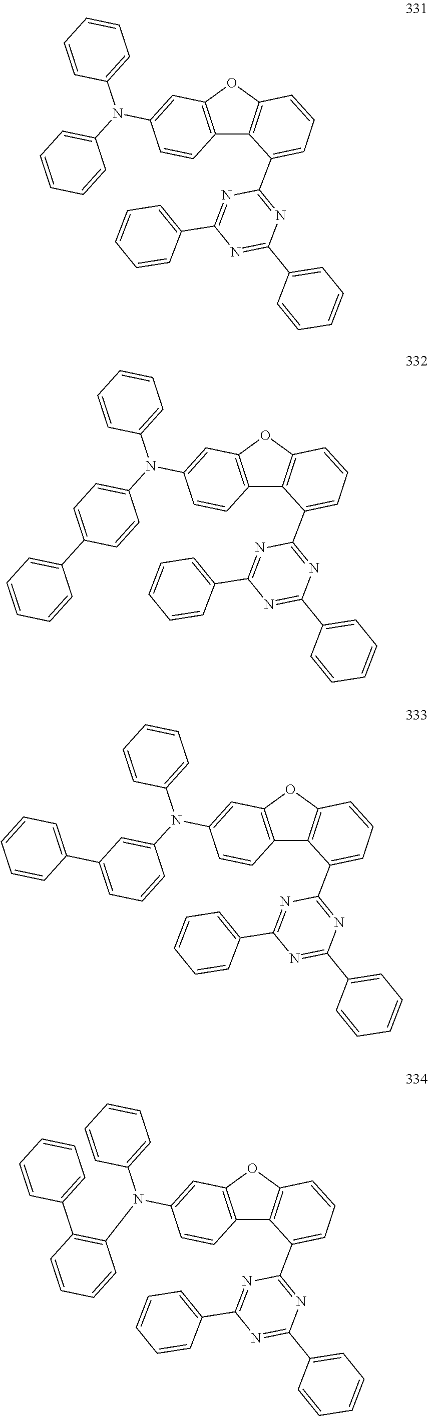 [SMILES: c1ccc(-c2nc(-c3ccccc3)nc(-c3cccc4oc5cc(N(c6ccccc6)c6ccccc6)ccc5c34)n2)cc1 ; c1ccc(-c2ccc(N(c3ccccc3)c3ccc4c(c3)oc3cccc(-c5nc(-c6ccccc6)nc(-c6ccccc6)n5)c34)cc2)cc1 ; c1ccc(-c2cccc(N(c3ccccc3)c3ccc4c(c3)oc3cccc(-c5nc(-c6ccccc6)nc(-c6ccccc6)n5)c34)c2)cc1 ; c1ccc(-c2nc(-c3ccccc3)nc(-c3cccc4oc5cc(N(c6ccccc6)c6ccccc6-c6ccccc6)ccc5c34)n2)cc1]

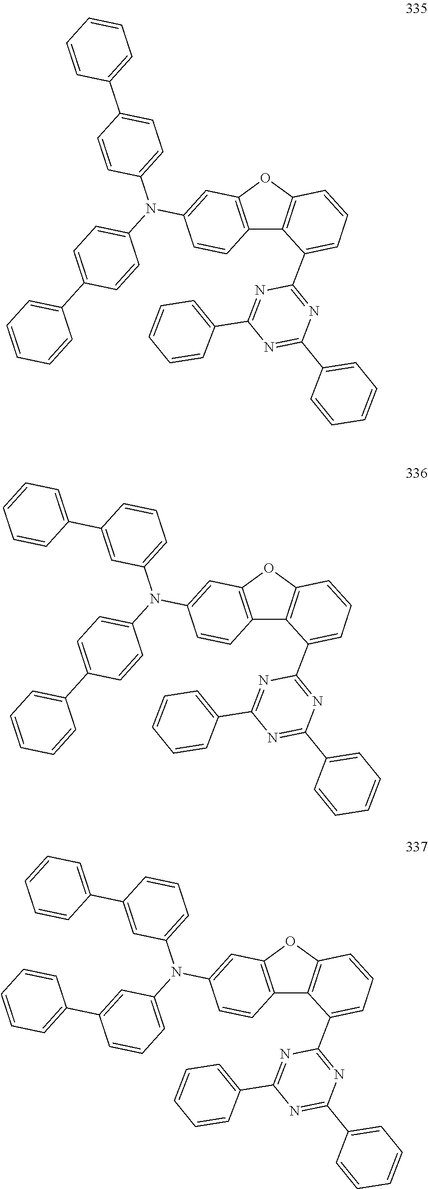 [SMILES: c1ccc(-c2ccc(N(c3ccc(-c4ccccc4)cc3)c3ccc4c(c3)oc3cccc(-c5nc(-c6ccccc6)nc(-c6ccccc6)n5)c34)cc2)cc1 ; c1ccc(-c2ccc(N(c3cccc(-c4ccccc4)c3)c3ccc4c(c3)oc3cccc(-c5nc(-c6ccccc6)nc(-c6ccccc6)n5)c34)cc2)cc1 ; c1ccc(-c2cccc(N(c3cccc(-c4ccccc4)c3)c3ccc4c(c3)oc3cccc(-c5nc(-c6ccccc6)nc(-c6ccccc6)n5)c34)c2)cc1]

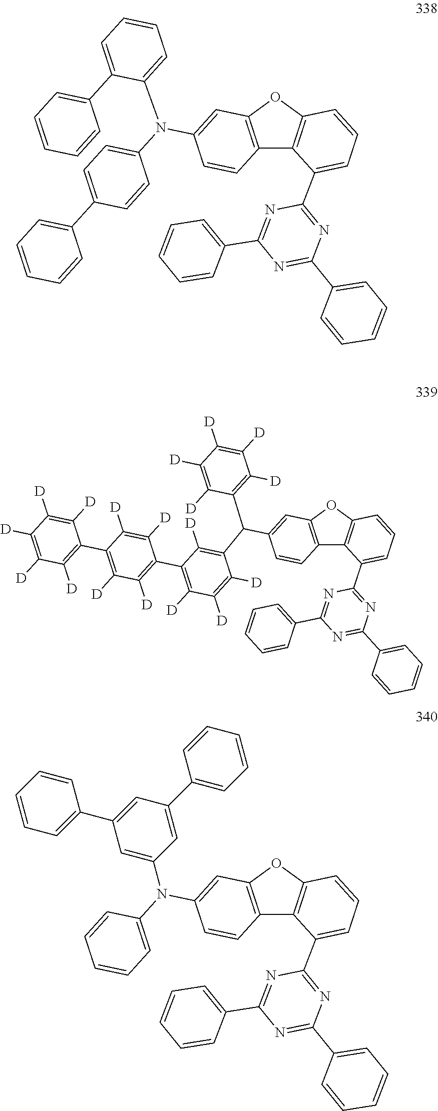 [SMILES: c1ccc(-c2ccc(N(c3ccc4c(c3)oc3cccc(-c5nc(-c6ccccc6)nc(-c6ccccc6)n5)c34)c3ccccc3-c3ccccc3)cc2)cc1 ; [2H]c1c([2H])c([2H])c(-c2c([2H])c([2H])c(-c3c([2H])c([2H])c([2H])c(C(c4ccc5c(c4)oc4cccc(-c6nc(-c7ccccc7)nc(-c7ccccc7)n6)c45)c4c([2H])c([2H])c([2H])c([2H])c4[2H])c3[2H])c([2H])c2[2H])c([2H])c1[2H] ; c1ccc(-c2cc(-c3ccccc3)cc(N(c3ccccc3)c3ccc4c(c3)oc3cccc(-c5nc(-c6ccccc6)nc(-c6ccccc6)n5)c34)c2)cc1]

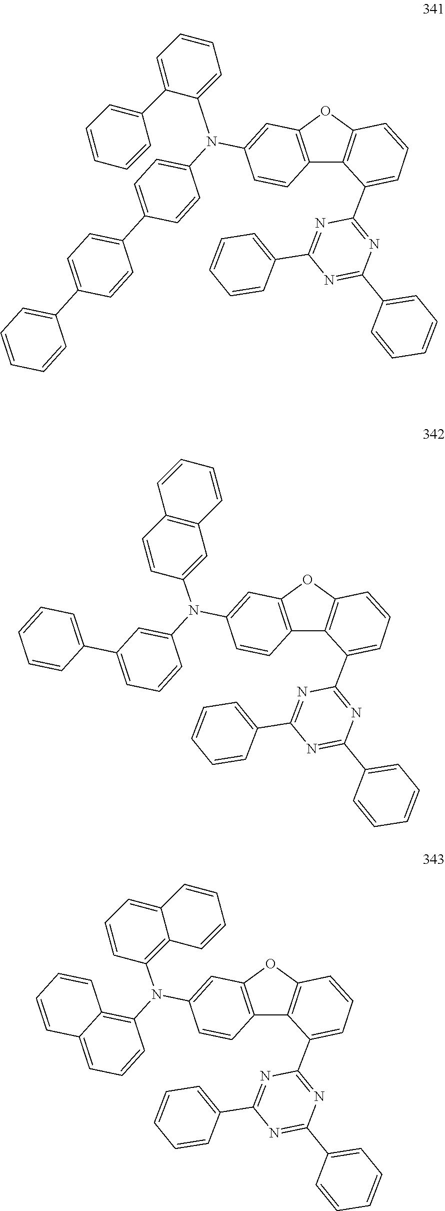 [SMILES: c1ccc(-c2ccc(-c3ccc(N(c4ccc5c(c4)oc4cccc(-c6nc(-c7ccccc7)nc(-c7ccccc7)n6)c45)c4ccccc4-c4ccccc4)cc3)cc2)cc1 ; c1ccc(-c2cccc(N(c3ccc4ccccc4c3)c3ccc4c(c3)oc3cccc(-c5nc(-c6ccccc6)nc(-c6ccccc6)n5)c34)c2)cc1 ; c1ccc(-c2nc(-c3ccccc3)nc(-c3cccc4oc5cc(N(c6cccc7ccccc67)c6cccc7ccccc67)ccc5c34)n2)cc1]

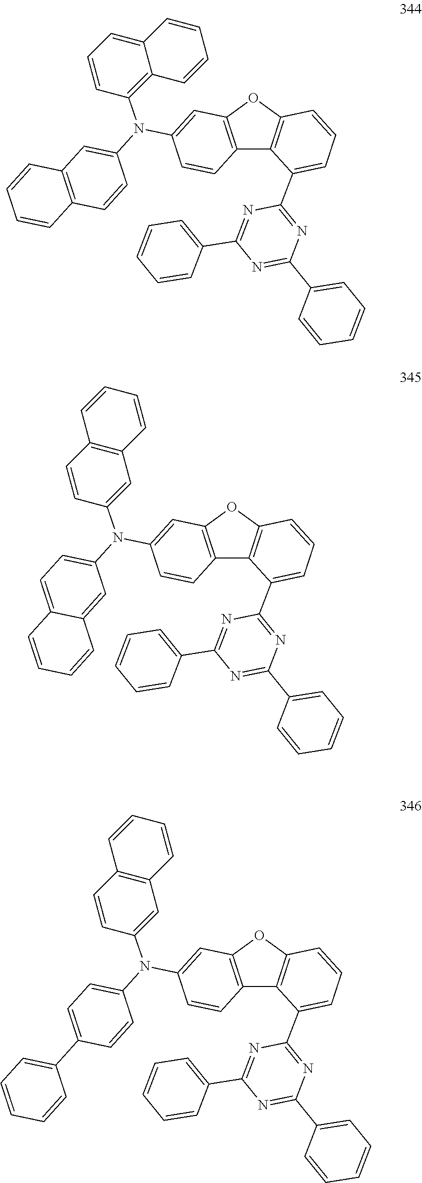 [SMILES: c1ccc(-c2nc(-c3ccccc3)nc(-c3cccc4oc5cc(N(c6ccc7ccccc7c6)c6cccc7ccccc67)ccc5c34)n2)cc1 ; c1ccc(-c2nc(-c3ccccc3)nc(-c3cccc4oc5cc(N(c6ccc7ccccc7c6)c6ccc7ccccc7c6)ccc5c34)n2)cc1 ; c1ccc(-c2ccc(N(c3ccc4ccccc4c3)c3ccc4c(c3)oc3cccc(-c5nc(-c6ccccc6)nc(-c6ccccc6)n5)c34)cc2)cc1]

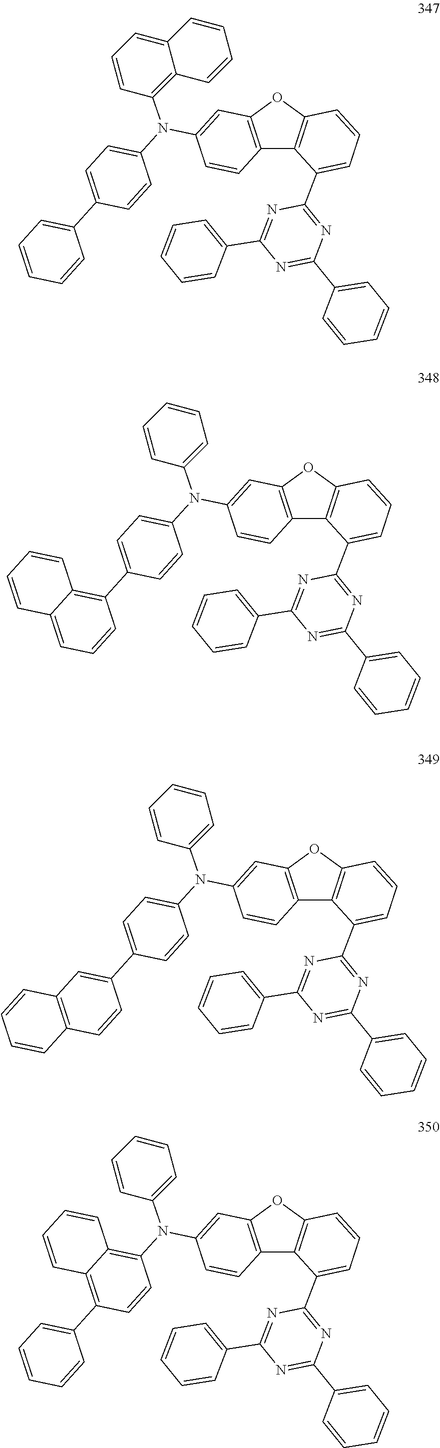 [SMILES: c1ccc(-c2ccc(N(c3ccc4c(c3)oc3cccc(-c5nc(-c6ccccc6)nc(-c6ccccc6)n5)c34)c3cccc4ccccc34)cc2)cc1 ; c1ccc(-c2nc(-c3ccccc3)nc(-c3cccc4oc5cc(N(c6ccccc6)c6ccc(-c7cccc8ccccc78)cc6)ccc5c34)n2)cc1 ; c1ccc(-c2nc(-c3ccccc3)nc(-c3cccc4oc5cc(N(c6ccccc6)c6ccc(-c7ccc8ccccc8c7)cc6)ccc5c34)n2)cc1 ; c1ccc(-c2nc(-c3ccccc3)nc(-c3cccc4oc5cc(N(c6ccccc6)c6ccc(-c7ccccc7)c7ccccc67)ccc5c34)n2)cc1]

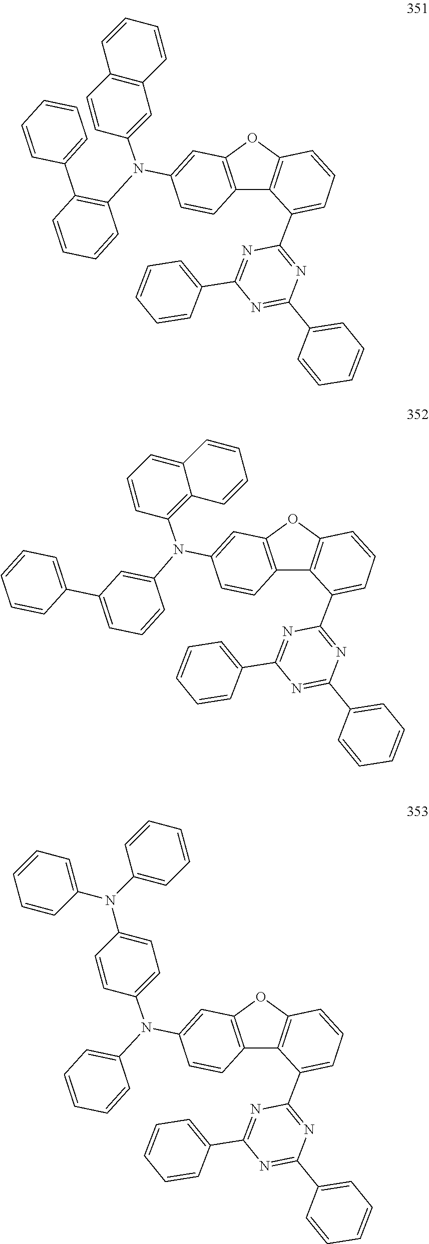 [SMILES: c1ccc(-c2nc(-c3ccccc3)nc(-c3cccc4oc5cc(N(c6ccc7ccccc7c6)c6ccccc6-c6ccccc6)ccc5c34)n2)cc1 ; c1ccc(-c2cccc(N(c3ccc4c(c3)oc3cccc(-c5nc(-c6ccccc6)nc(-c6ccccc6)n5)c34)c3cccc4ccccc34)c2)cc1 ; c1ccc(-c2nc(-c3ccccc3)nc(-c3cccc4oc5cc(N(c6ccccc6)c6ccc(N(c7ccccc7)c7ccccc7)cc6)ccc5c34)n2)cc1]

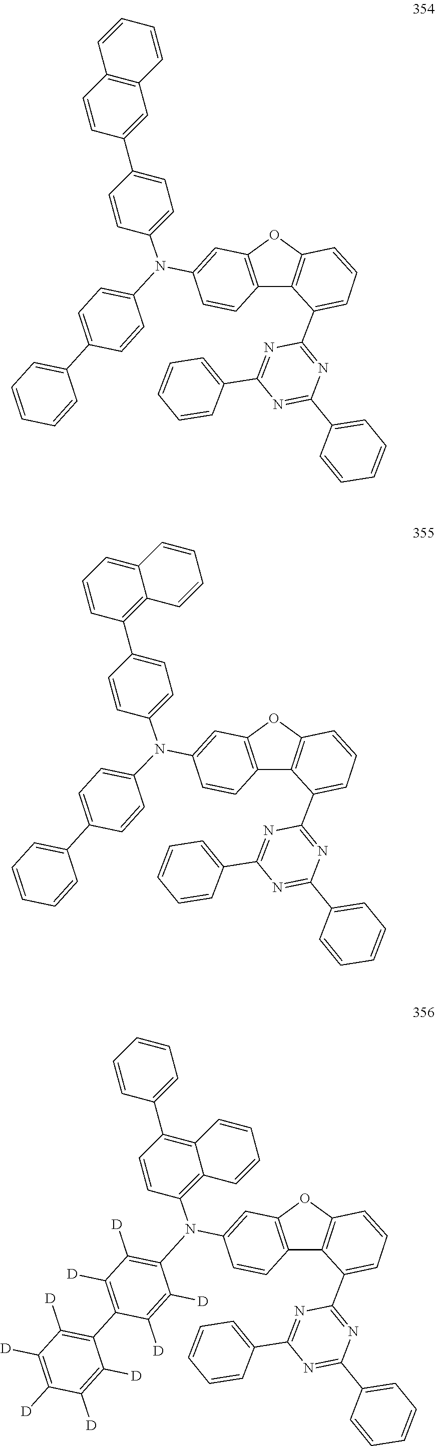 [SMILES: c1ccc(-c2ccc(N(c3ccc(-c4ccc5ccccc5c4)cc3)c3ccc4c(c3)oc3cccc(-c5nc(-c6ccccc6)nc(-c6ccccc6)n5)c34)cc2)cc1 ; c1ccc(-c2ccc(N(c3ccc(-c4cccc5ccccc45)cc3)c3ccc4c(c3)oc3cccc(-c5nc(-c6ccccc6)nc(-c6ccccc6)n5)c34)cc2)cc1 ; [2H]c1c([2H])c([2H])c(-c2c([2H])c([2H])c(N(c3ccc4c(c3)oc3cccc(-c5nc(-c6ccccc6)nc(-c6ccccc6)n5)c34)c3ccc(-c4ccccc4)c4ccccc34)c([2H])c2[2H])c([2H])c1[2H]]

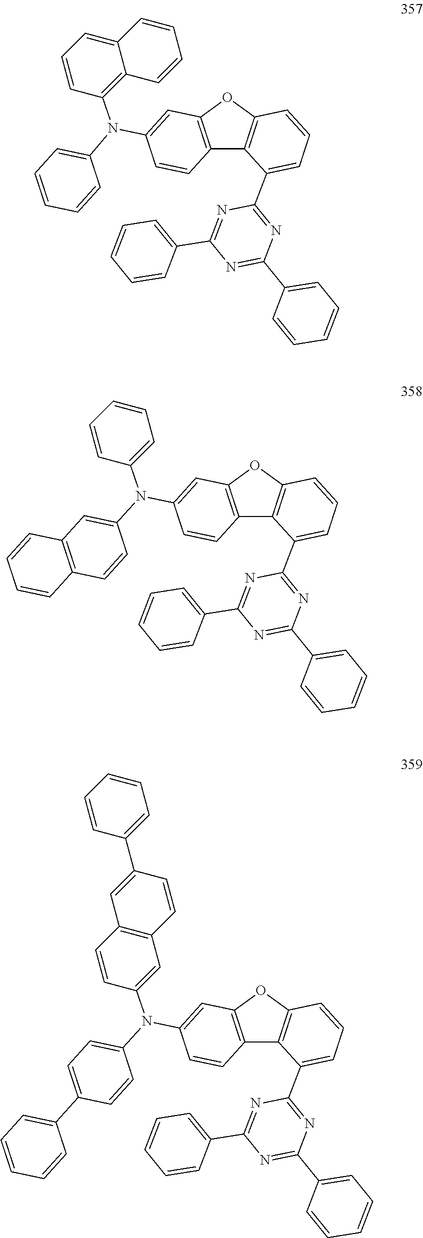 [SMILES: c1ccc(-c2nc(-c3ccccc3)nc(-c3cccc4oc5cc(N(c6ccccc6)c6cccc7ccccc67)ccc5c34)n2)cc1 ; c1ccc(-c2nc(-c3ccccc3)nc(-c3cccc4oc5cc(N(c6ccccc6)c6ccc7ccccc7c6)ccc5c34)n2)cc1 ; c1ccc(-c2ccc(N(c3ccc4cc(-c5ccccc5)ccc4c3)c3ccc4c(c3)oc3cccc(-c5nc(-c6ccccc6)nc(-c6ccccc6)n5)c34)cc2)cc1]

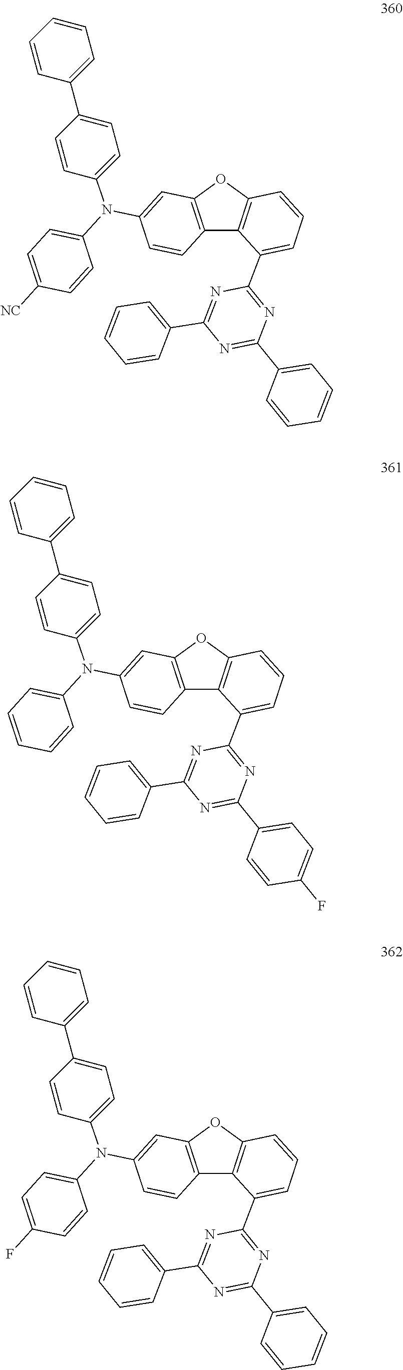 [SMILES: N#Cc1ccc(N(c2ccc(-c3ccccc3)cc2)c2ccc3c(c2)oc2cccc(-c4nc(-c5ccccc5)nc(-c5ccccc5)n4)c23)cc1 ; Fc1ccc(-c2nc(-c3ccccc3)nc(-c3cccc4oc5cc(N(c6ccccc6)c6ccc(-c7ccccc7)cc6)ccc5c34)n2)cc1 ; Fc1ccc(N(c2ccc(-c3ccccc3)cc2)c2ccc3c(c2)oc2cccc(-c4nc(-c5ccccc5)nc(-c5ccccc5)n4)c23)cc1]

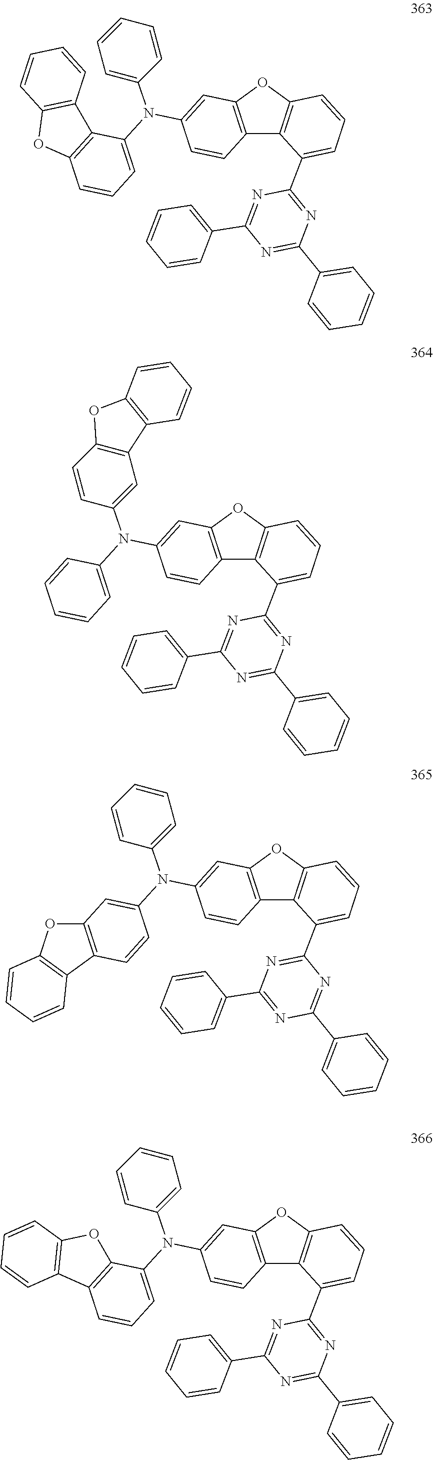 [SMILES: c1ccc(-c2nc(-c3ccccc3)nc(-c3cccc4oc5cc(N(c6ccccc6)c6cccc7oc8ccccc8c67)ccc5c34)n2)cc1 ; c1ccc(-c2nc(-c3ccccc3)nc(-c3cccc4oc5cc(N(c6ccccc6)c6ccc7oc8ccccc8c7c6)ccc5c34)n2)cc1 ; c1ccc(-c2nc(-c3ccccc3)nc(-c3cccc4oc5cc(N(c6ccccc6)c6ccc7c(c6)oc6ccccc67)ccc5c34)n2)cc1 ; c1ccc(-c2nc(-c3ccccc3)nc(-c3cccc4oc5cc(N(c6ccccc6)c6cccc7c6oc6ccccc67)ccc5c34)n2)cc1]

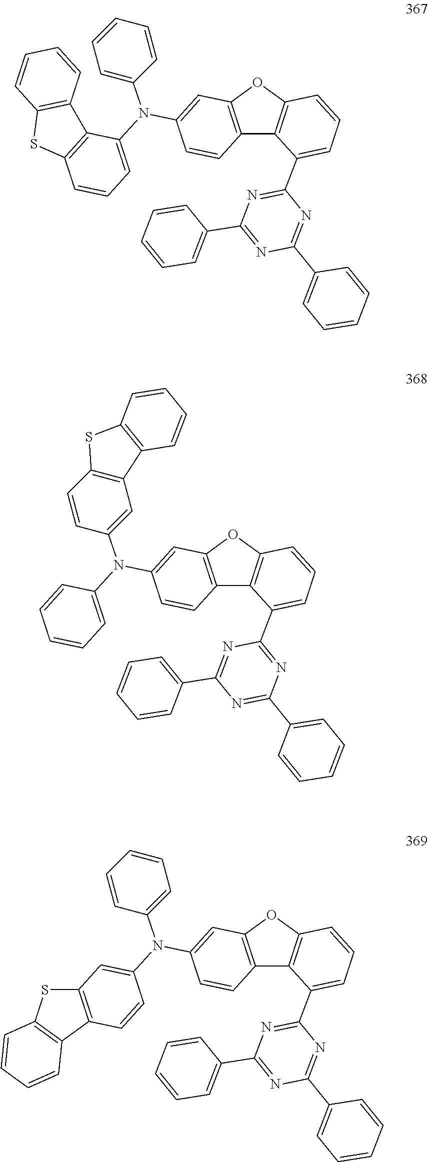 [SMILES: c1ccc(-c2nc(-c3ccccc3)nc(-c3cccc4oc5cc(N(c6ccccc6)c6cccc7sc8ccccc8c67)ccc5c34)n2)cc1 ; c1ccc(-c2nc(-c3ccccc3)nc(-c3cccc4oc5cc(N(c6ccccc6)c6ccc7sc8ccccc8c7c6)ccc5c34)n2)cc1 ; c1ccc(-c2nc(-c3ccccc3)nc(-c3cccc4oc5cc(N(c6ccccc6)c6ccc7c(c6)sc6ccccc67)ccc5c34)n2)cc1]

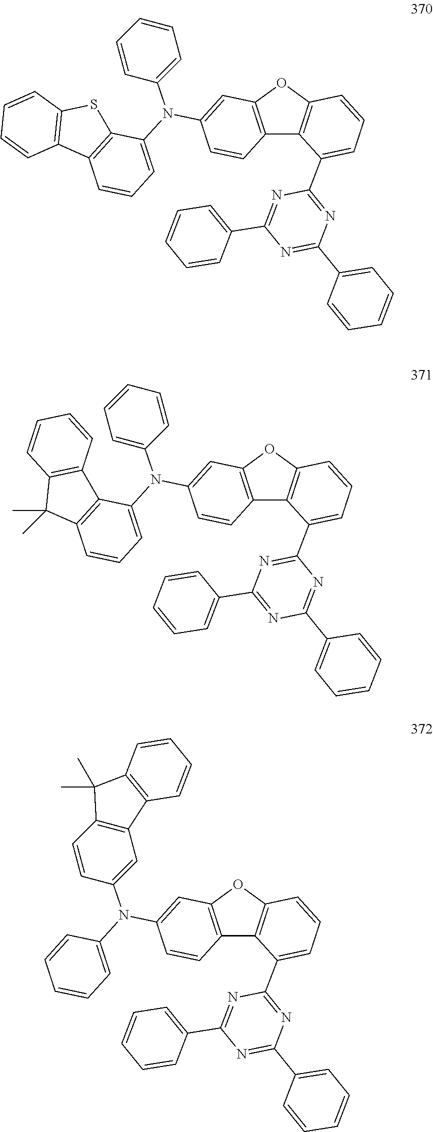 [SMILES: c1ccc(-c2nc(-c3ccccc3)nc(-c3cccc4oc5cc(N(c6ccccc6)c6cccc7c6sc6ccccc67)ccc5c34)n2)cc1 ; CC1(C)c2ccccc2-c2c(N(c3ccccc3)c3ccc4c(c3)oc3cccc(-c5nc(-c6ccccc6)nc(-c6ccccc6)n5)c34)cccc21 ; CC1(C)c2ccccc2-c2cc(N(c3ccccc3)c3ccc4c(c3)oc3cccc(-c5nc(-c6ccccc6)nc(-c6ccccc6)n5)c34)ccc21]

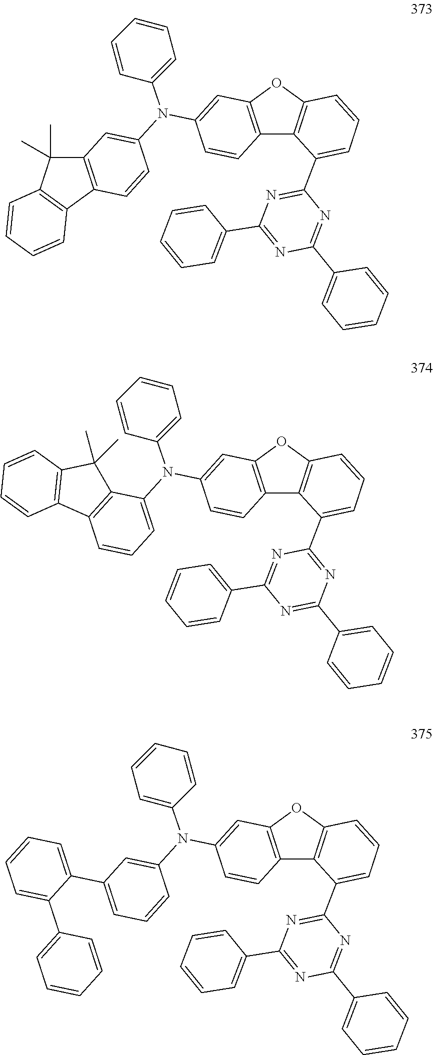 [SMILES: CC1(C)c2ccccc2-c2ccc(N(c3ccccc3)c3ccc4c(c3)oc3cccc(-c5nc(-c6ccccc6)nc(-c6ccccc6)n5)c34)cc21 ; CC1(C)c2ccccc2-c2cccc(N(c3ccccc3)c3ccc4c(c3)oc3cccc(-c5nc(-c6ccccc6)nc(-c6ccccc6)n5)c34)c21 ; c1ccc(-c2nc(-c3ccccc3)nc(-c3cccc4oc5cc(N(c6ccccc6)c6cccc(-c7ccccc7-c7ccccc7)c6)ccc5c34)n2)cc1]

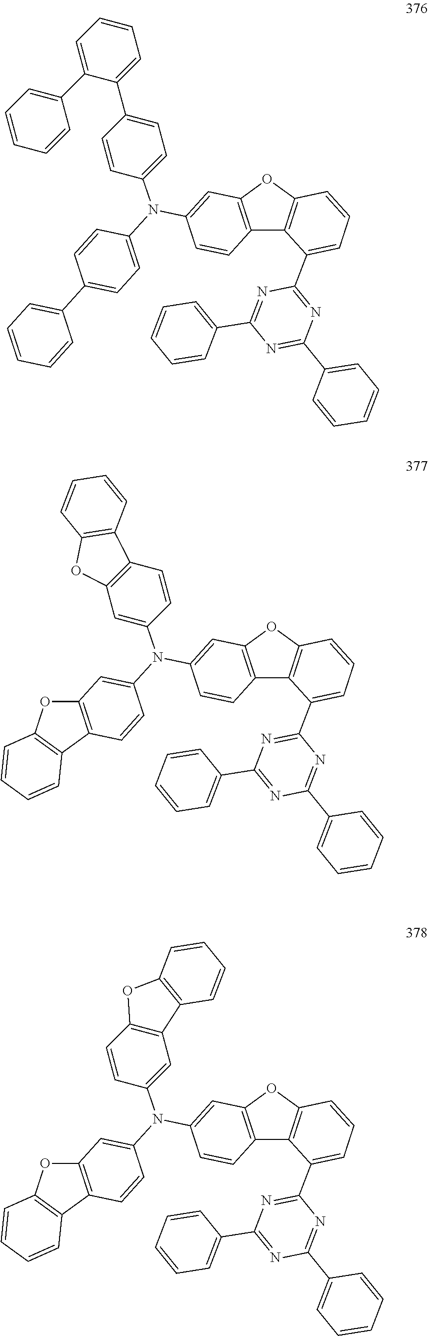 [SMILES: c1ccc(-c2ccc(N(c3ccc(-c4ccccc4-c4ccccc4)cc3)c3ccc4c(c3)oc3cccc(-c5nc(-c6ccccc6)nc(-c6ccccc6)n5)c34)cc2)cc1 ; c1ccc(-c2nc(-c3ccccc3)nc(-c3cccc4oc5cc(N(c6ccc7c(c6)oc6ccccc67)c6ccc7c(c6)oc6ccccc67)ccc5c34)n2)cc1 ; c1ccc(-c2nc(-c3ccccc3)nc(-c3cccc4oc5cc(N(c6ccc7c(c6)oc6ccccc67)c6ccc7oc8ccccc8c7c6)ccc5c34)n2)cc1]

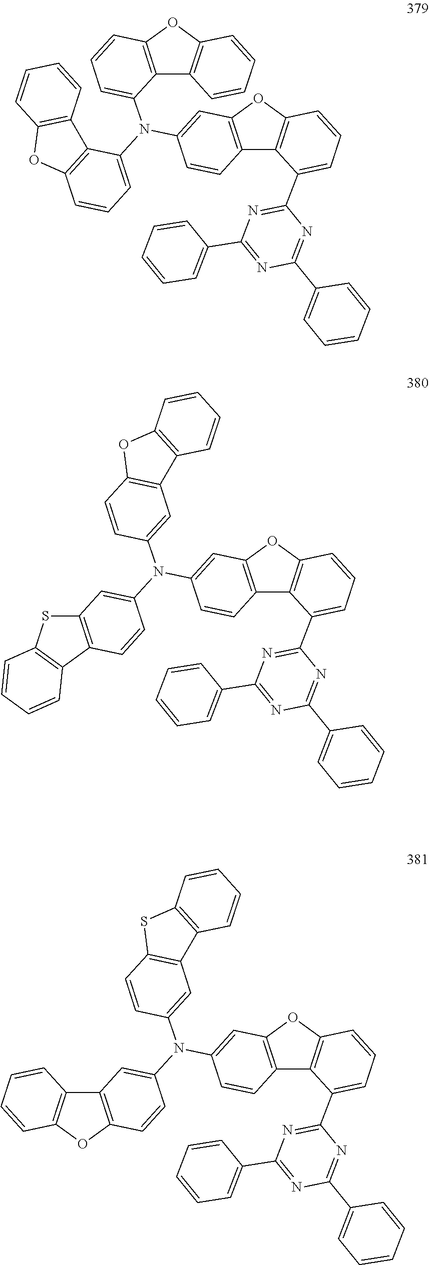 [SMILES: c1ccc(-c2nc(-c3ccccc3)nc(-c3cccc4oc5cc(N(c6cccc7oc8ccccc8c67)c6cccc7oc8ccccc8c67)ccc5c34)n2)cc1 ; c1ccc(-c2nc(-c3ccccc3)nc(-c3cccc4oc5cc(N(c6ccc7c(c6)sc6ccccc67)c6ccc7oc8ccccc8c7c6)ccc5c34)n2)cc1 ; c1ccc(-c2nc(-c3ccccc3)nc(-c3cccc4oc5cc(N(c6ccc7oc8ccccc8c7c6)c6ccc7sc8ccccc8c7c6)ccc5c34)n2)cc1]

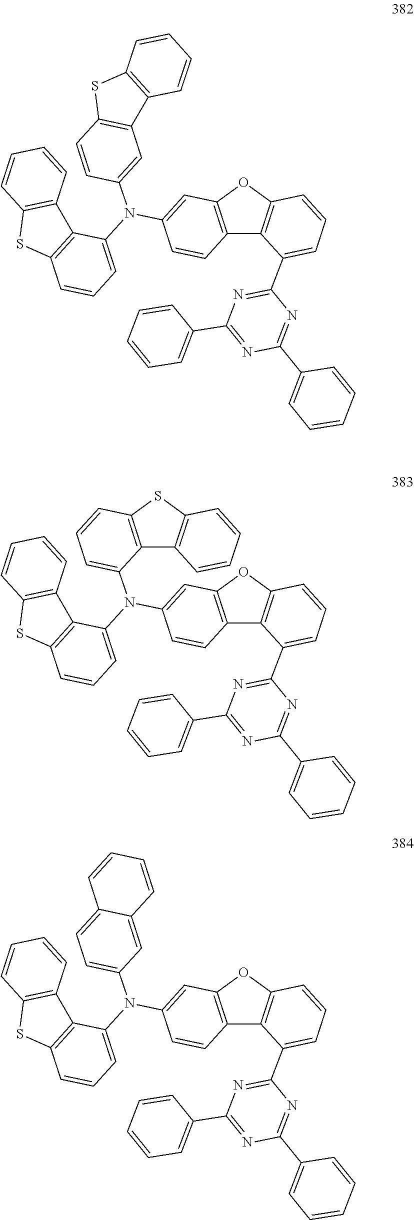 [SMILES: c1ccc(-c2nc(-c3ccccc3)nc(-c3cccc4oc5cc(N(c6ccc7sc8ccccc8c7c6)c6cccc7sc8ccccc8c67)ccc5c34)n2)cc1 ; c1ccc(-c2nc(-c3ccccc3)nc(-c3cccc4oc5cc(N(c6cccc7sc8ccccc8c67)c6cccc7sc8ccccc8c67)ccc5c34)n2)cc1 ; c1ccc(-c2nc(-c3ccccc3)nc(-c3cccc4oc5cc(N(c6ccc7ccccc7c6)c6cccc7sc8ccccc8c67)ccc5c34)n2)cc1]

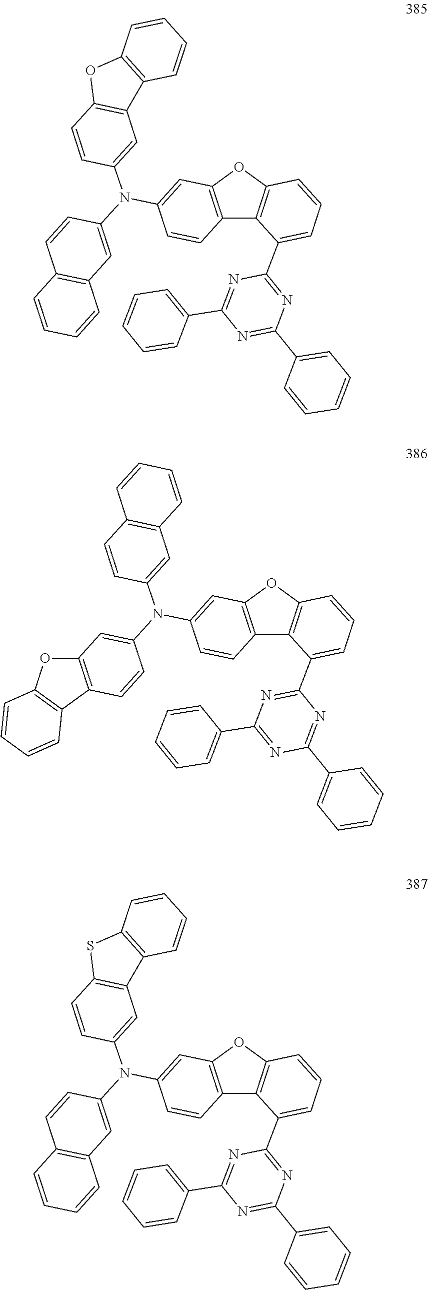 [SMILES: c1ccc(-c2nc(-c3ccccc3)nc(-c3cccc4oc5cc(N(c6ccc7ccccc7c6)c6ccc7oc8ccccc8c7c6)ccc5c34)n2)cc1 ; c1ccc(-c2nc(-c3ccccc3)nc(-c3cccc4oc5cc(N(c6ccc7ccccc7c6)c6ccc7c(c6)oc6ccccc67)ccc5c34)n2)cc1 ; c1ccc(-c2nc(-c3ccccc3)nc(-c3cccc4oc5cc(N(c6ccc7ccccc7c6)c6ccc7sc8ccccc8c7c6)ccc5c34)n2)cc1]

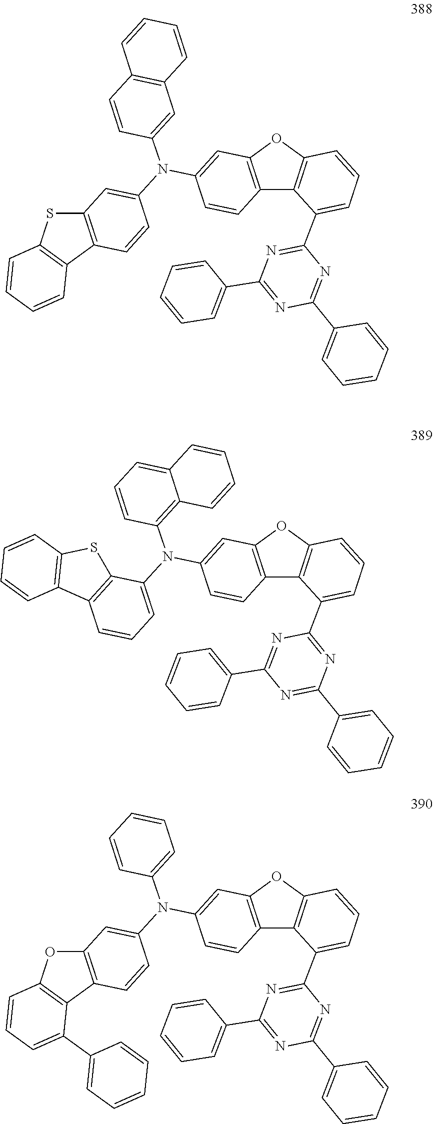 [SMILES: c1ccc(-c2nc(-c3ccccc3)nc(-c3cccc4oc5cc(N(c6ccc7ccccc7c6)c6ccc7c(c6)sc6ccccc67)ccc5c34)n2)cc1 ; c1ccc(-c2nc(-c3ccccc3)nc(-c3cccc4oc5cc(N(c6cccc7ccccc67)c6cccc7c6sc6ccccc67)ccc5c34)n2)cc1 ; c1ccc(-c2nc(-c3ccccc3)nc(-c3cccc4oc5cc(N(c6ccccc6)c6ccc7c(c6)oc6cccc(-c8ccccc8)c67)ccc5c34)n2)cc1]

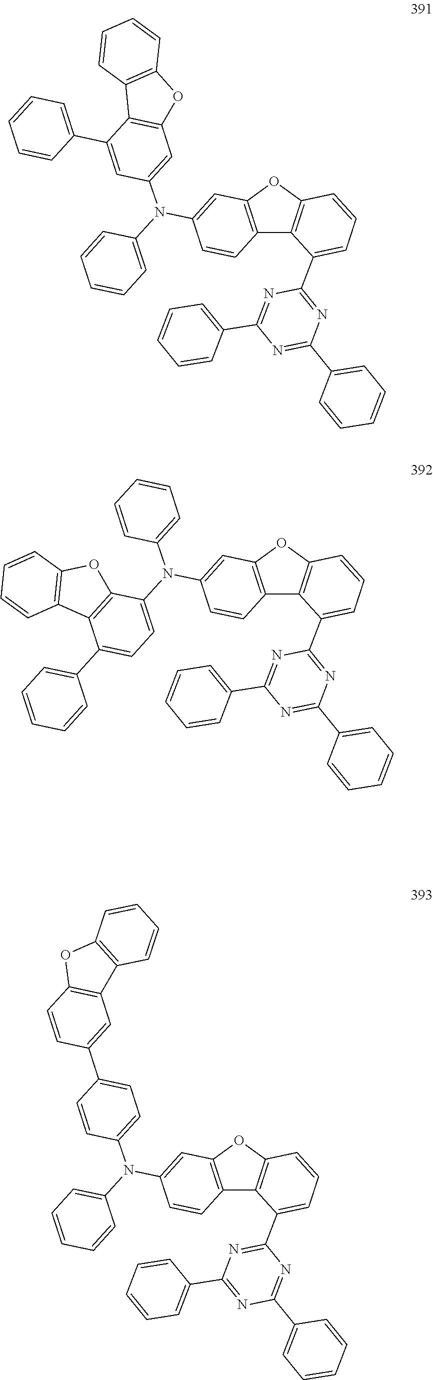 [SMILES: c1ccc(-c2nc(-c3ccccc3)nc(-c3cccc4oc5cc(N(c6ccccc6)c6cc(-c7ccccc7)c7c(c6)oc6ccccc67)ccc5c34)n2)cc1 ; c1ccc(-c2nc(-c3ccccc3)nc(-c3cccc4oc5cc(N(c6ccccc6)c6ccc(-c7ccccc7)c7c6oc6ccccc67)ccc5c34)n2)cc1 ; c1ccc(-c2nc(-c3ccccc3)nc(-c3cccc4oc5cc(N(c6ccccc6)c6ccc(-c7ccc8oc9ccccc9c8c7)cc6)ccc5c34)n2)cc1]

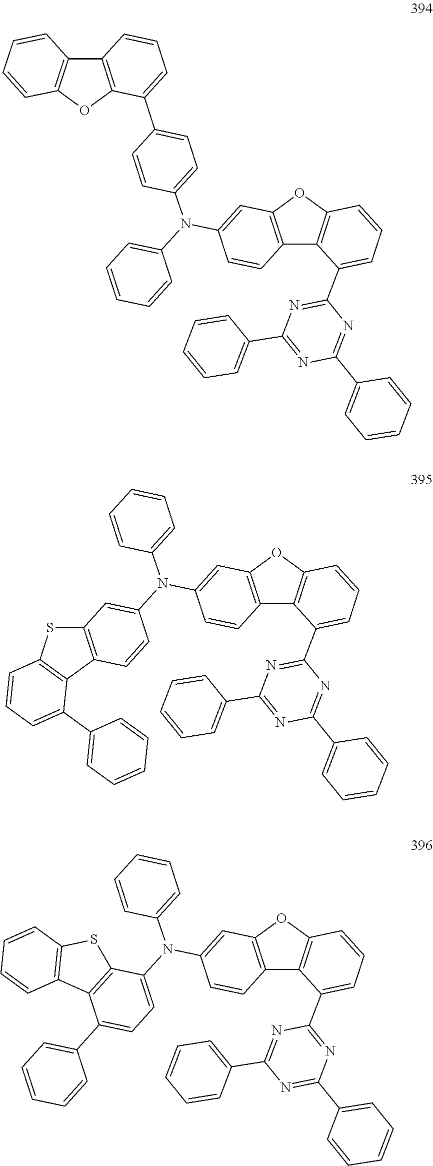 [SMILES: c1ccc(-c2nc(-c3ccccc3)nc(-c3cccc4oc5cc(N(c6ccccc6)c6ccc(-c7cccc8c7oc7ccccc78)cc6)ccc5c34)n2)cc1 ; c1ccc(-c2nc(-c3ccccc3)nc(-c3cccc4oc5cc(N(c6ccccc6)c6ccc7c(c6)sc6cccc(-c8ccccc8)c67)ccc5c34)n2)cc1 ; c1ccc(-c2nc(-c3ccccc3)nc(-c3cccc4oc5cc(N(c6ccccc6)c6ccc(-c7ccccc7)c7c6sc6ccccc67)ccc5c34)n2)cc1]

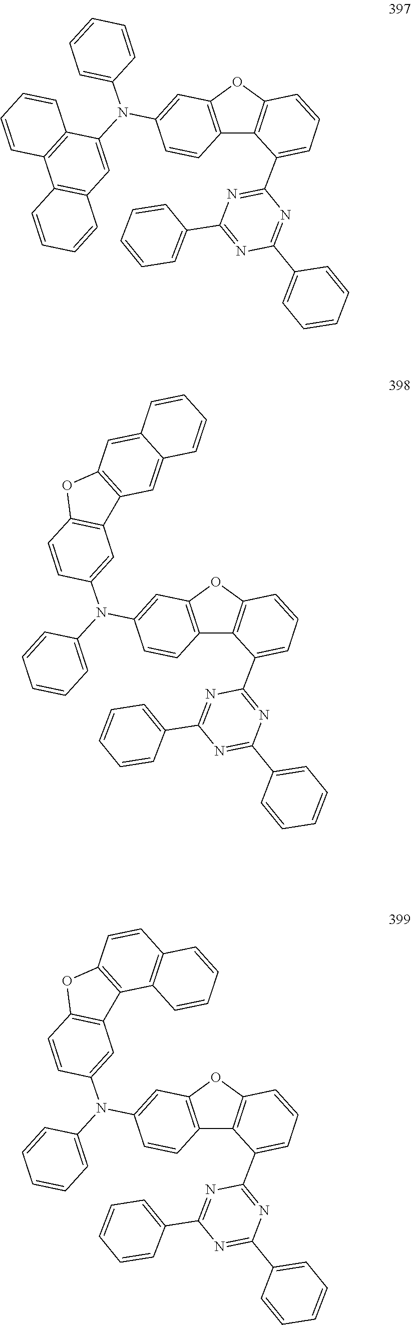 [SMILES: c1ccc(-c2nc(-c3ccccc3)nc(-c3cccc4oc5cc(N(c6ccccc6)c6cc7ccccc7c7ccccc67)ccc5c34)n2)cc1 ; c1ccc(-c2nc(-c3ccccc3)nc(-c3cccc4oc5cc(N(c6ccccc6)c6ccc7oc8cc9ccccc9cc8c7c6)ccc5c34)n2)cc1 ; c1ccc(-c2nc(-c3ccccc3)nc(-c3cccc4oc5cc(N(c6ccccc6)c6ccc7oc8ccc9ccccc9c8c7c6)ccc5c34)n2)cc1]

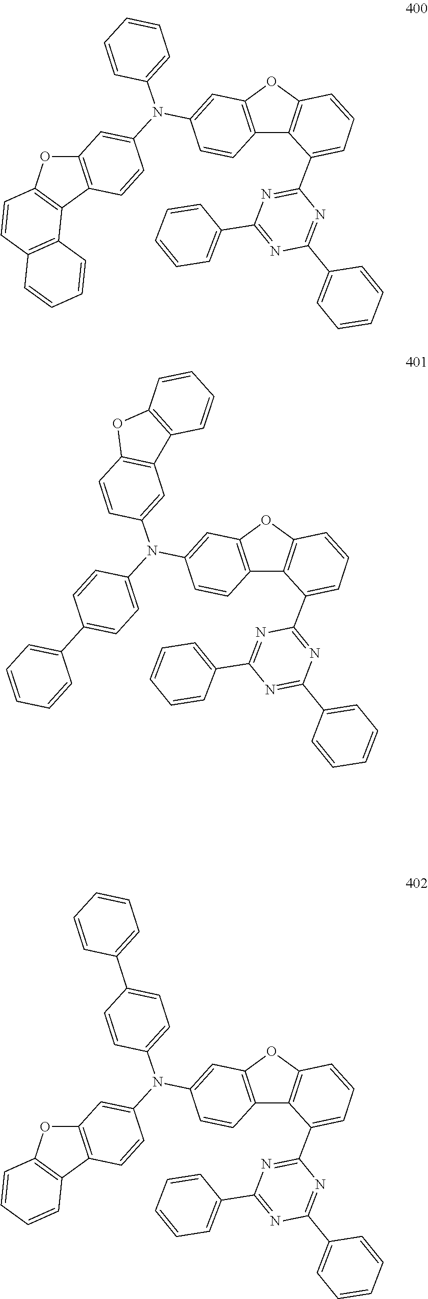 [SMILES: c1ccc(-c2nc(-c3ccccc3)nc(-c3cccc4oc5cc(N(c6ccccc6)c6ccc7c(c6)oc6ccc8ccccc8c67)ccc5c34)n2)cc1 ; c1ccc(-c2ccc(N(c3ccc4c(c3)oc3cccc(-c5nc(-c6ccccc6)nc(-c6ccccc6)n5)c34)c3ccc4oc5ccccc5c4c3)cc2)cc1 ; c1ccc(-c2ccc(N(c3ccc4c(c3)oc3ccccc34)c3ccc4c(c3)oc3cccc(-c5nc(-c6ccccc6)nc(-c6ccccc6)n5)c34)cc2)cc1]

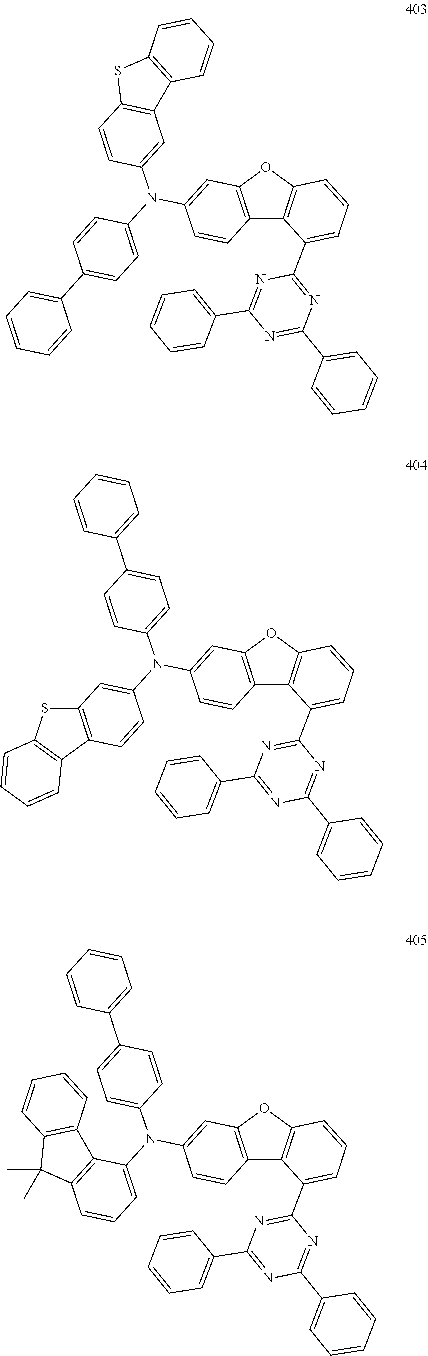 [SMILES: c1ccc(-c2ccc(N(c3ccc4c(c3)oc3cccc(-c5nc(-c6ccccc6)nc(-c6ccccc6)n5)c34)c3ccc4sc5ccccc5c4c3)cc2)cc1 ; c1ccc(-c2ccc(N(c3ccc4c(c3)oc3cccc(-c5nc(-c6ccccc6)nc(-c6ccccc6)n5)c34)c3ccc4c(c3)sc3ccccc34)cc2)cc1 ; CC1(C)c2ccccc2-c2c(N(c3ccc(-c4ccccc4)cc3)c3ccc4c(c3)oc3cccc(-c5nc(-c6ccccc6)nc(-c6ccccc6)n5)c34)cccc21]

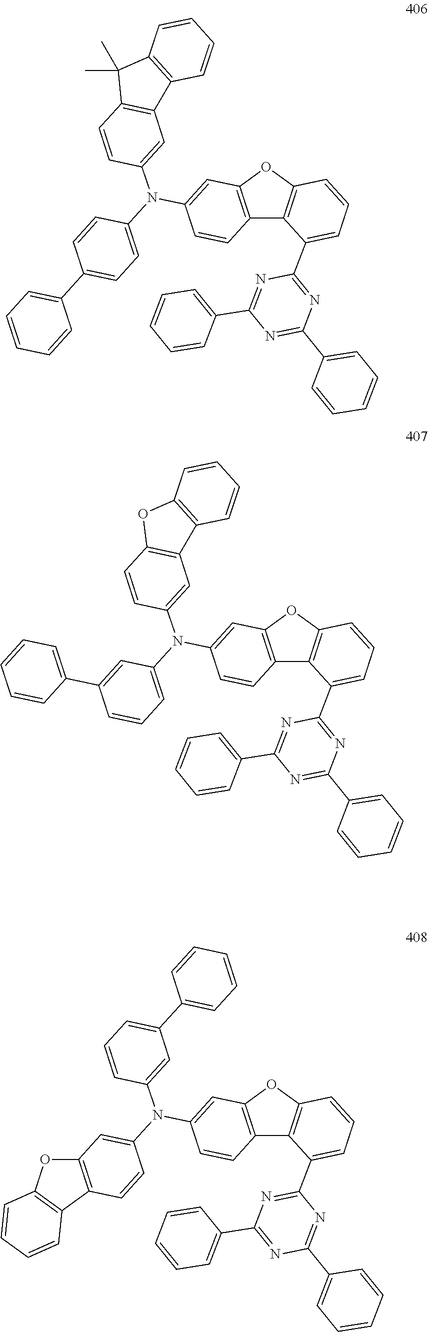 [SMILES: CC1(C)c2ccccc2-c2cc(N(c3ccc(-c4ccccc4)cc3)c3ccc4c(c3)oc3cccc(-c5nc(-c6ccccc6)nc(-c6ccccc6)n5)c34)ccc21 ; c1ccc(-c2cccc(N(c3ccc4c(c3)oc3cccc(-c5nc(-c6ccccc6)nc(-c6ccccc6)n5)c34)c3ccc4oc5ccccc5c4c3)c2)cc1 ; c1ccc(-c2cccc(N(c3ccc4c(c3)oc3ccccc34)c3ccc4c(c3)oc3cccc(-c5nc(-c6ccccc6)nc(-c6ccccc6)n5)c34)c2)cc1]

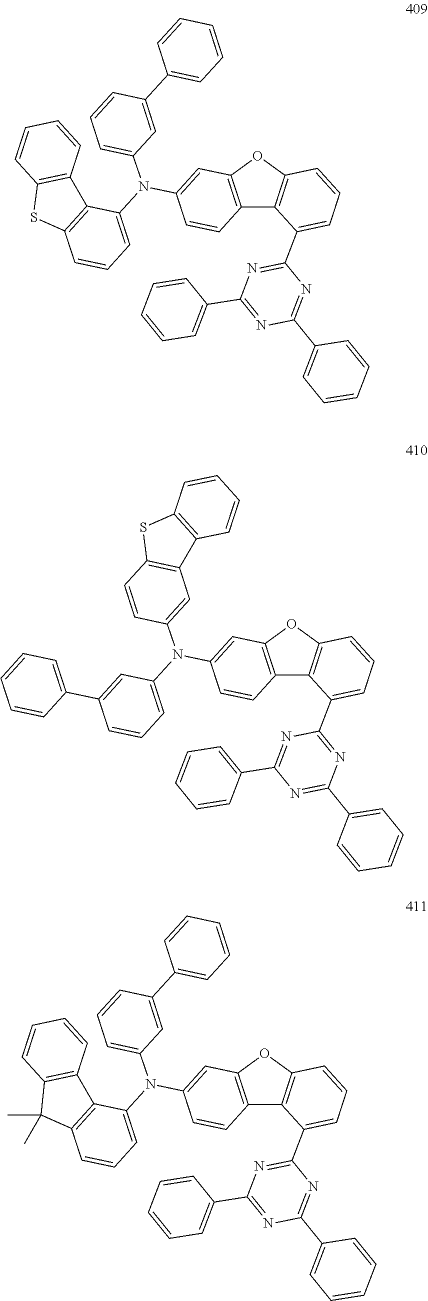 [SMILES: c1ccc(-c2cccc(N(c3ccc4c(c3)oc3cccc(-c5nc(-c6ccccc6)nc(-c6ccccc6)n5)c34)c3cccc4sc5ccccc5c34)c2)cc1 ; c1ccc(-c2cccc(N(c3ccc4c(c3)oc3cccc(-c5nc(-c6ccccc6)nc(-c6ccccc6)n5)c34)c3ccc4sc5ccccc5c4c3)c2)cc1 ; CC1(C)c2ccccc2-c2c(N(c3cccc(-c4ccccc4)c3)c3ccc4c(c3)oc3cccc(-c5nc(-c6ccccc6)nc(-c6ccccc6)n5)c34)cccc21]

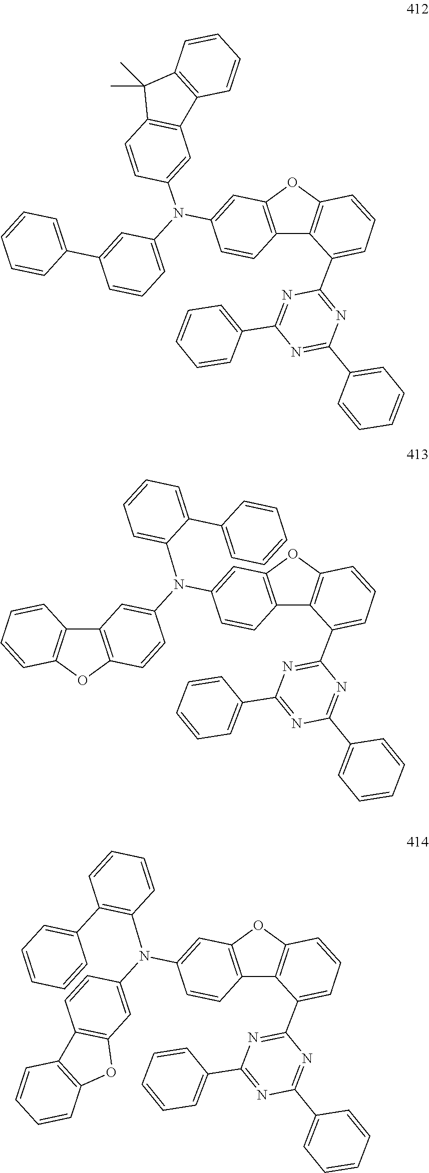 [SMILES: CC1(C)c2ccccc2-c2cc(N(c3cccc(-c4ccccc4)c3)c3ccc4c(c3)oc3cccc(-c5nc(-c6ccccc6)nc(-c6ccccc6)n5)c34)ccc21 ; c1ccc(-c2nc(-c3ccccc3)nc(-c3cccc4oc5cc(N(c6ccc7oc8ccccc8c7c6)c6ccccc6-c6ccccc6)ccc5c34)n2)cc1 ; c1ccc(-c2nc(-c3ccccc3)nc(-c3cccc4oc5cc(N(c6ccc7c(c6)oc6ccccc67)c6ccccc6-c6ccccc6)ccc5c34)n2)cc1]

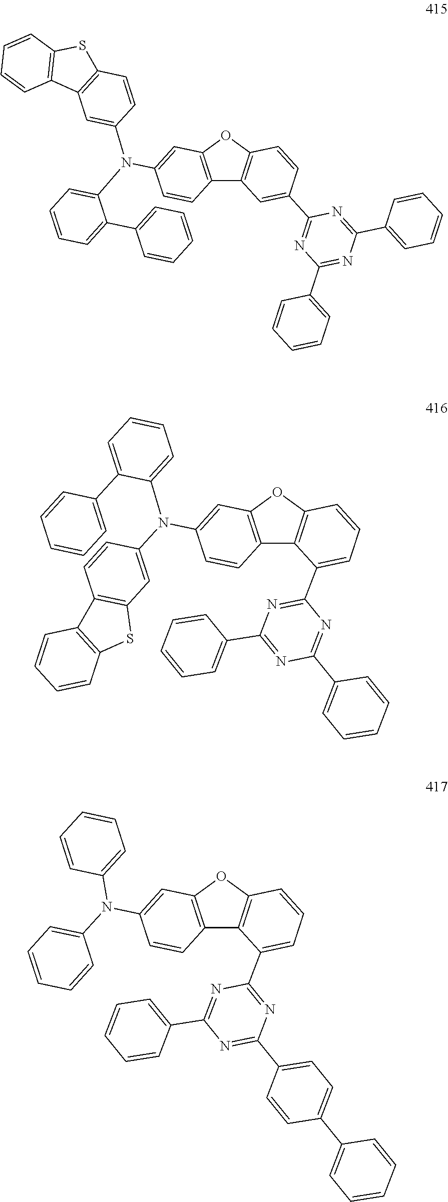 [SMILES: c1ccc(-c2nc(-c3ccccc3)nc(-c3ccc4oc5cc(N(c6ccc7sc8ccccc8c7c6)c6ccccc6-c6ccccc6)ccc5c4c3)n2)cc1 ; c1ccc(-c2nc(-c3ccccc3)nc(-c3cccc4oc5cc(N(c6ccc7c(c6)sc6ccccc67)c6ccccc6-c6ccccc6)ccc5c34)n2)cc1 ; c1ccc(-c2ccc(-c3nc(-c4ccccc4)nc(-c4cccc5oc6cc(N(c7ccccc7)c7ccccc7)ccc6c45)n3)cc2)cc1]

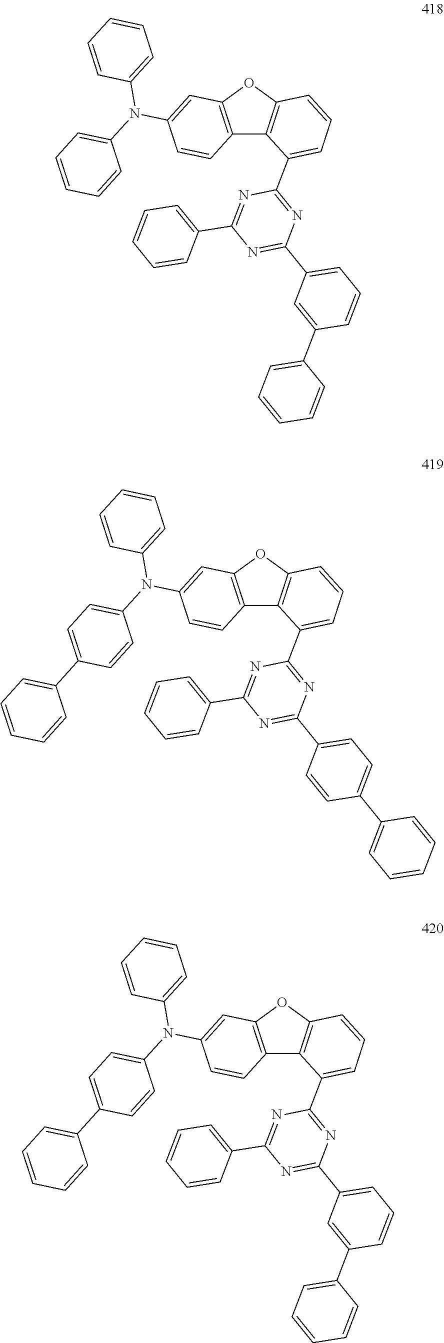 [SMILES: c1ccc(-c2cccc(-c3nc(-c4ccccc4)nc(-c4cccc5oc6cc(N(c7ccccc7)c7ccccc7)ccc6c45)n3)c2)cc1 ; c1ccc(-c2ccc(-c3nc(-c4ccccc4)nc(-c4cccc5oc6cc(N(c7ccccc7)c7ccc(-c8ccccc8)cc7)ccc6c45)n3)cc2)cc1 ; c1ccc(-c2ccc(N(c3ccccc3)c3ccc4c(c3)oc3cccc(-c5nc(-c6ccccc6)nc(-c6cccc(-c7ccccc7)c6)n5)c34)cc2)cc1]

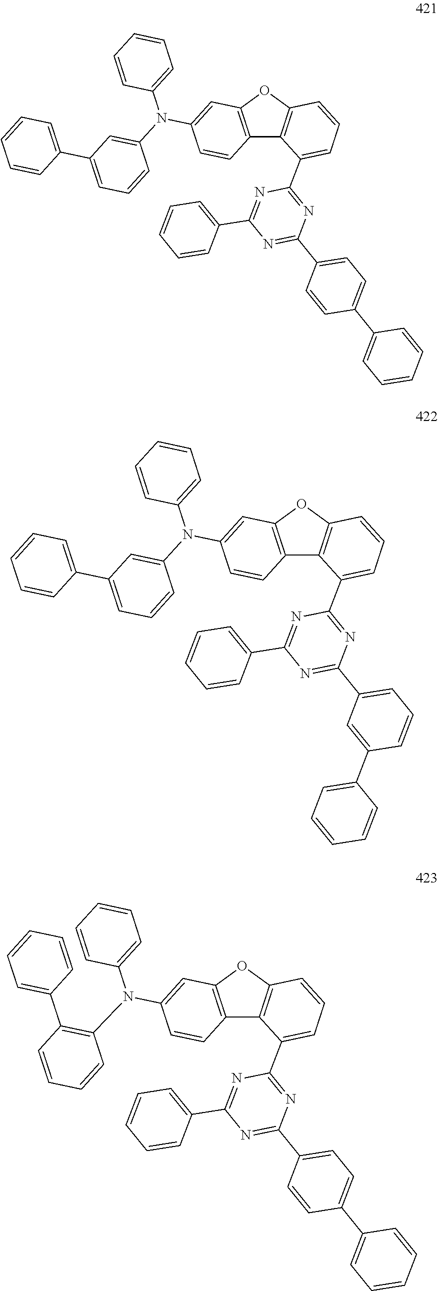 [SMILES: c1ccc(-c2ccc(-c3nc(-c4ccccc4)nc(-c4cccc5oc6cc(N(c7ccccc7)c7cccc(-c8ccccc8)c7)ccc6c45)n3)cc2)cc1 ; c1ccc(-c2cccc(-c3nc(-c4ccccc4)nc(-c4cccc5oc6cc(N(c7ccccc7)c7cccc(-c8ccccc8)c7)ccc6c45)n3)c2)cc1 ; c1ccc(-c2ccc(-c3nc(-c4ccccc4)nc(-c4cccc5oc6cc(N(c7ccccc7)c7ccccc7-c7ccccc7)ccc6c45)n3)cc2)cc1]

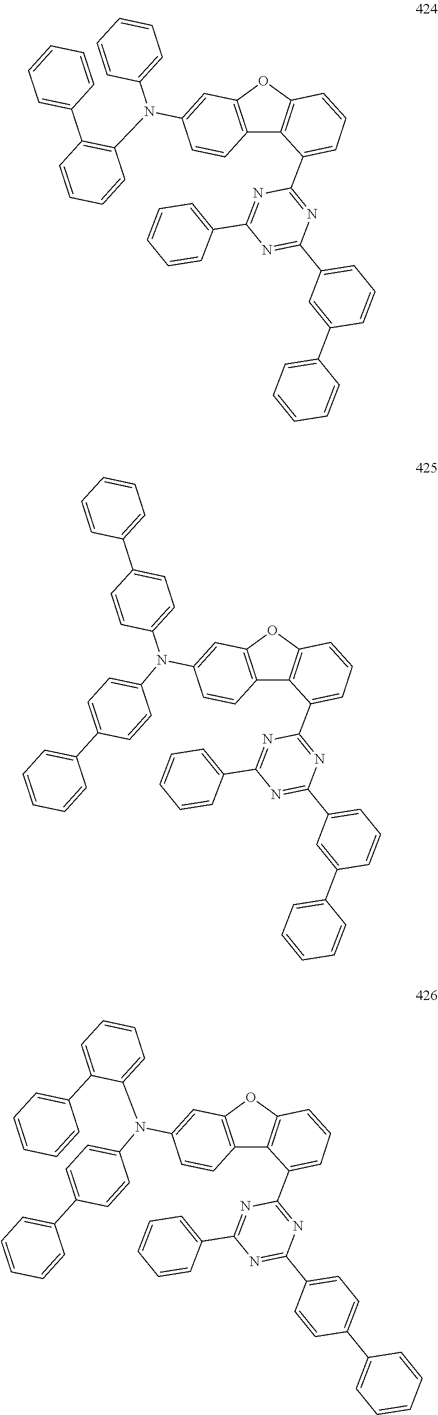 [SMILES: c1ccc(-c2cccc(-c3nc(-c4ccccc4)nc(-c4cccc5oc6cc(N(c7ccccc7)c7ccccc7-c7ccccc7)ccc6c45)n3)c2)cc1 ; c1ccc(-c2ccc(N(c3ccc(-c4ccccc4)cc3)c3ccc4c(c3)oc3cccc(-c5nc(-c6ccccc6)nc(-c6cccc(-c7ccccc7)c6)n5)c34)cc2)cc1 ; c1ccc(-c2ccc(-c3nc(-c4ccccc4)nc(-c4cccc5oc6cc(N(c7ccc(-c8ccccc8)cc7)c7ccccc7-c7ccccc7)ccc6c45)n3)cc2)cc1]

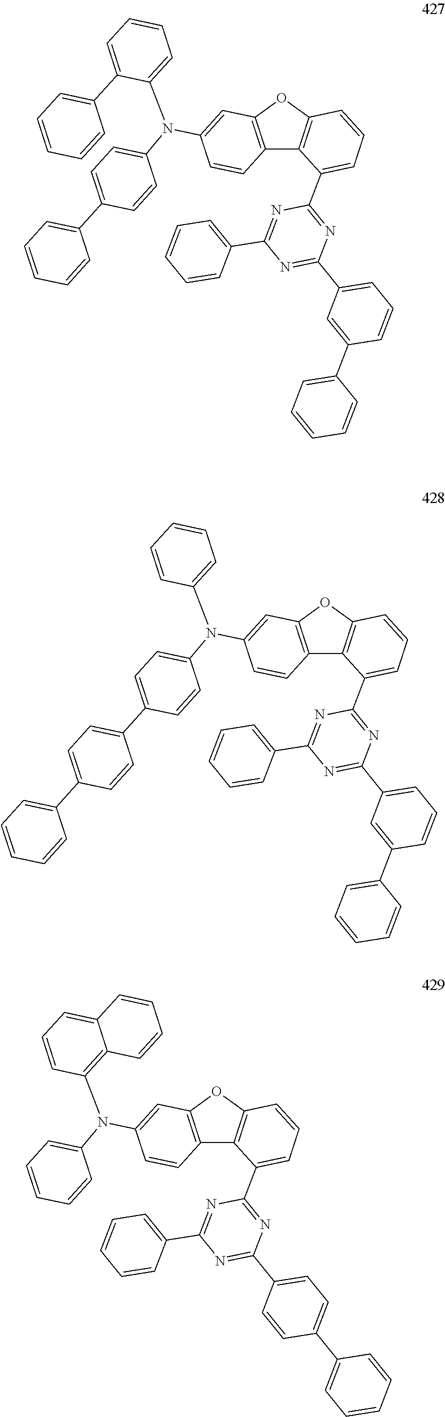 [SMILES: c1ccc(-c2ccc(N(c3ccc4c(c3)oc3cccc(-c5nc(-c6ccccc6)nc(-c6cccc(-c7ccccc7)c6)n5)c34)c3ccccc3-c3ccccc3)cc2)cc1 ; c1ccc(-c2ccc(-c3ccc(N(c4ccccc4)c4ccc5c(c4)oc4cccc(-c6nc(-c7ccccc7)nc(-c7cccc(-c8ccccc8)c7)n6)c45)cc3)cc2)cc1 ; c1ccc(-c2ccc(-c3nc(-c4ccccc4)nc(-c4cccc5oc6cc(N(c7ccccc7)c7cccc8ccccc78)ccc6c45)n3)cc2)cc1]

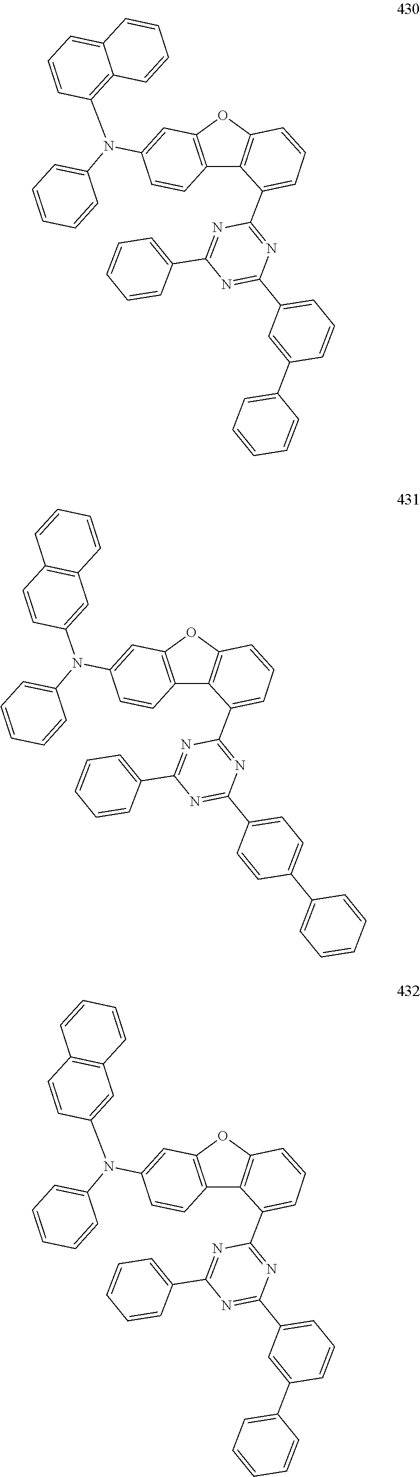 [SMILES: c1ccc(-c2cccc(-c3nc(-c4ccccc4)nc(-c4cccc5oc6cc(N(c7ccccc7)c7cccc8ccccc78)ccc6c45)n3)c2)cc1 ; c1ccc(-c2ccc(-c3nc(-c4ccccc4)nc(-c4cccc5oc6cc(N(c7ccccc7)c7ccc8ccccc8c7)ccc6c45)n3)cc2)cc1 ; c1ccc(-c2cccc(-c3nc(-c4ccccc4)nc(-c4cccc5oc6cc(N(c7ccccc7)c7ccc8ccccc8c7)ccc6c45)n3)c2)cc1]

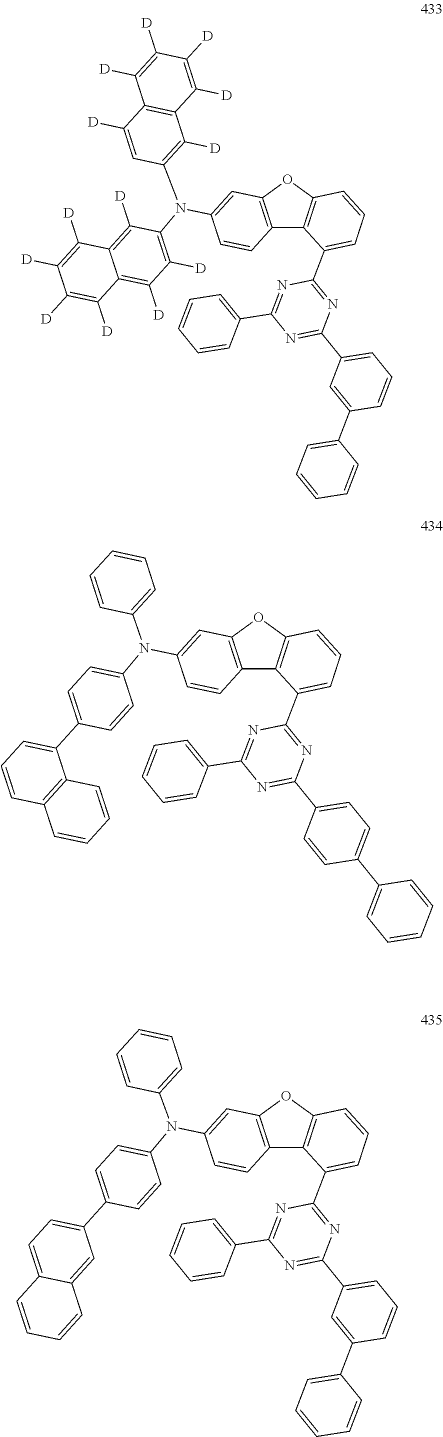 [SMILES: [2H]c1c([2H])c([2H])c2c([2H])c(N(c3ccc4c(c3)oc3cccc(-c5nc(-c6ccccc6)nc(-c6cccc(-c7ccccc7)c6)n5)c34)c3c([2H])c([2H])c4c([2H])c([2H])c([2H])c([2H])c4c3[2H])cc([2H])c2c1[2H] ; c1ccc(-c2ccc(-c3nc(-c4ccccc4)nc(-c4cccc5oc6cc(N(c7ccccc7)c7ccc(-c8cccc9ccccc89)cc7)ccc6c45)n3)cc2)cc1 ; c1ccc(-c2cccc(-c3nc(-c4ccccc4)nc(-c4cccc5oc6cc(N(c7ccccc7)c7ccc(-c8ccc9ccccc9c8)cc7)ccc6c45)n3)c2)cc1]

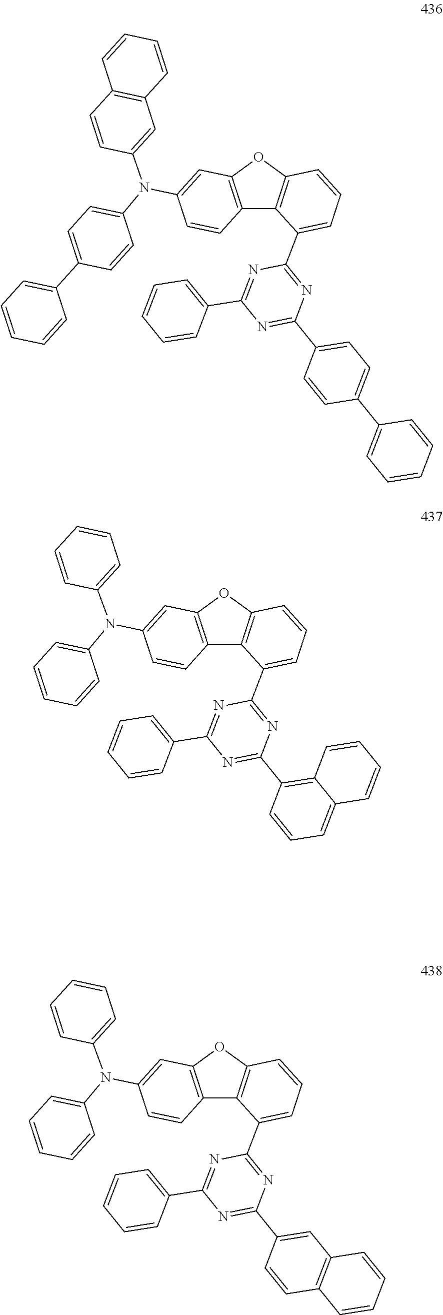 [SMILES: c1ccc(-c2ccc(-c3nc(-c4ccccc4)nc(-c4cccc5oc6cc(N(c7ccc(-c8ccccc8)cc7)c7ccc8ccccc8c7)ccc6c45)n3)cc2)cc1 ; c1ccc(-c2nc(-c3cccc4ccccc34)nc(-c3cccc4oc5cc(N(c6ccccc6)c6ccccc6)ccc5c34)n2)cc1 ; c1ccc(-c2nc(-c3ccc4ccccc4c3)nc(-c3cccc4oc5cc(N(c6ccccc6)c6ccccc6)ccc5c34)n2)cc1]

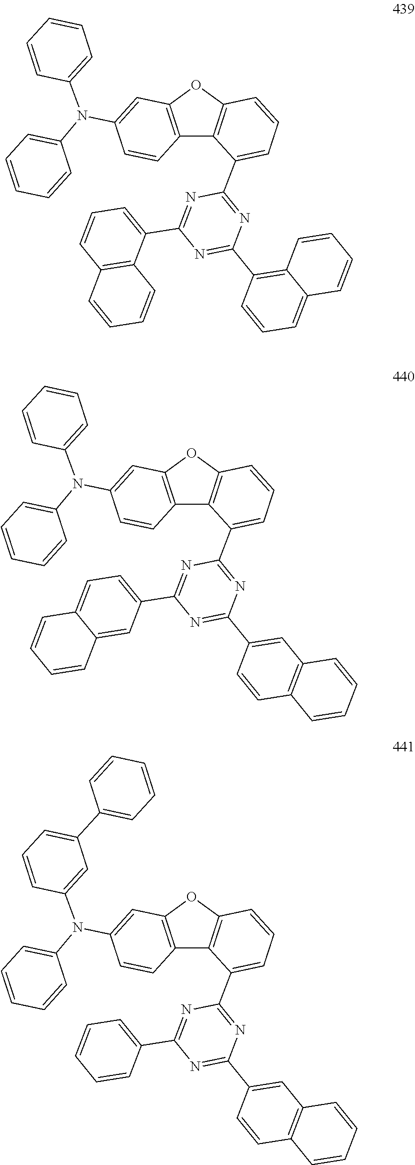 [SMILES: c1ccc(N(c2ccccc2)c2ccc3c(c2)oc2cccc(-c4nc(-c5cccc6ccccc56)nc(-c5cccc6ccccc56)n4)c23)cc1 ; c1ccc(N(c2ccccc2)c2ccc3c(c2)oc2cccc(-c4nc(-c5ccc6ccccc6c5)nc(-c5ccc6ccccc6c5)n4)c23)cc1 ; c1ccc(-c2cccc(N(c3ccccc3)c3ccc4c(c3)oc3cccc(-c5nc(-c6ccccc6)nc(-c6ccc7ccccc7c6)n5)c34)c2)cc1]

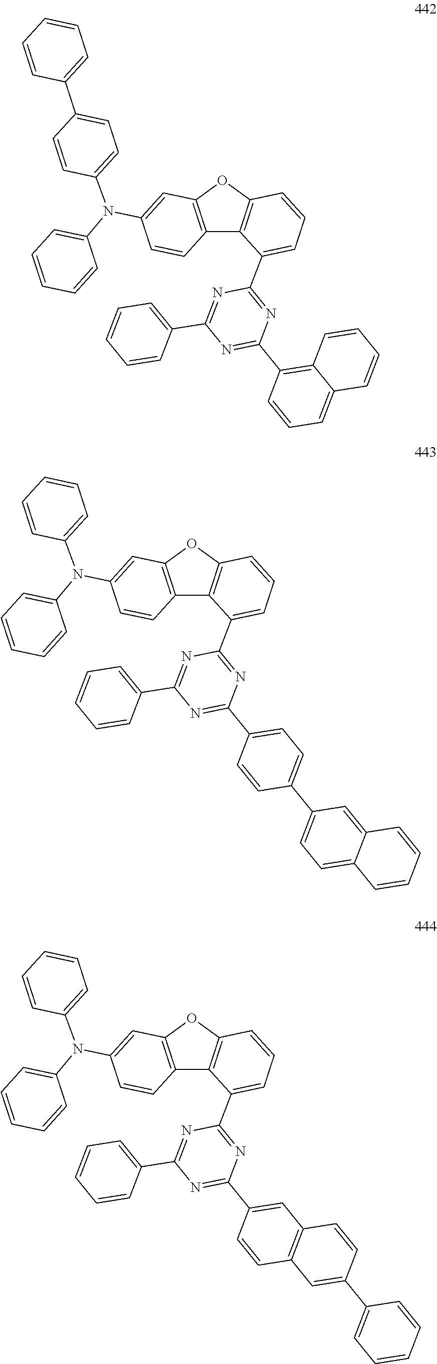 [SMILES: c1ccc(-c2ccc(N(c3ccccc3)c3ccc4c(c3)oc3cccc(-c5nc(-c6ccccc6)nc(-c6cccc7ccccc67)n5)c34)cc2)cc1 ; c1ccc(-c2nc(-c3ccc(-c4ccc5ccccc5c4)cc3)nc(-c3cccc4oc5cc(N(c6ccccc6)c6ccccc6)ccc5c34)n2)cc1 ; c1ccc(-c2ccc3cc(-c4nc(-c5ccccc5)nc(-c5cccc6oc7cc(N(c8ccccc8)c8ccccc8)ccc7c56)n4)ccc3c2)cc1]

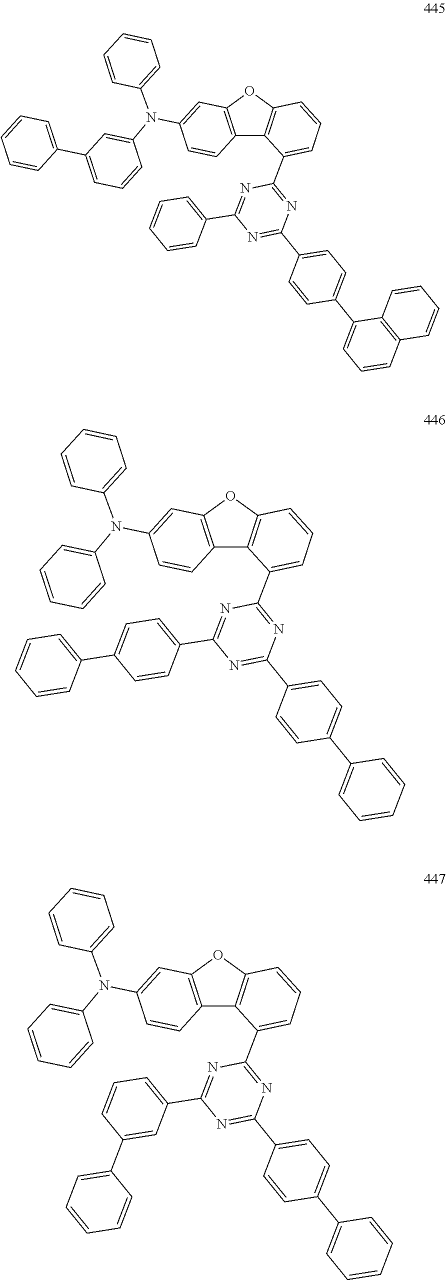 [SMILES: c1ccc(-c2cccc(N(c3ccccc3)c3ccc4c(c3)oc3cccc(-c5nc(-c6ccccc6)nc(-c6ccc(-c7cccc8ccccc78)cc6)n5)c34)c2)cc1 ; c1ccc(-c2ccc(-c3nc(-c4ccc(-c5ccccc5)cc4)nc(-c4cccc5oc6cc(N(c7ccccc7)c7ccccc7)ccc6c45)n3)cc2)cc1 ; c1ccc(-c2ccc(-c3nc(-c4cccc(-c5ccccc5)c4)nc(-c4cccc5oc6cc(N(c7ccccc7)c7ccccc7)ccc6c45)n3)cc2)cc1]

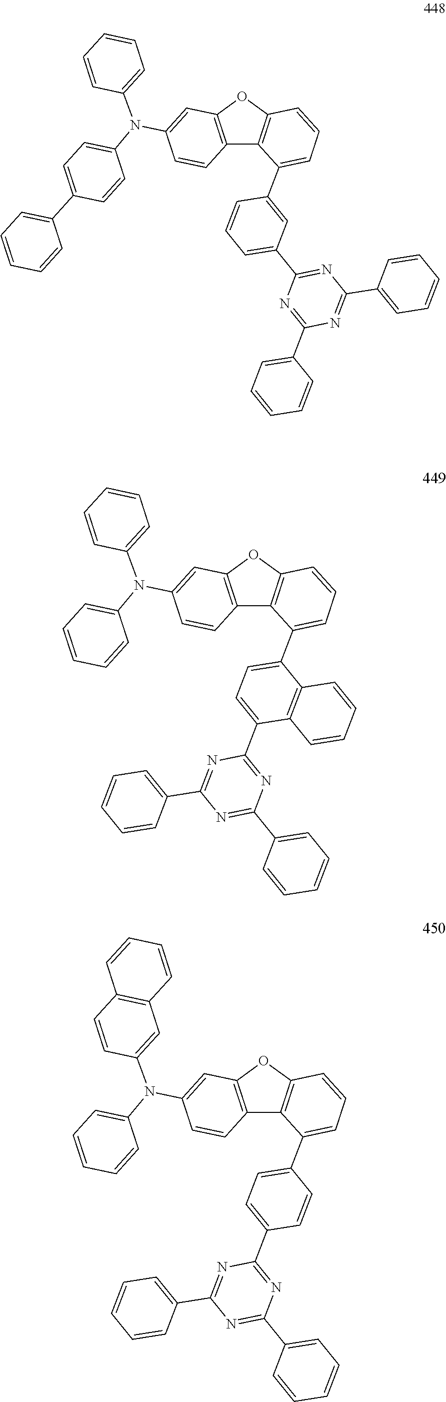 [SMILES: c1ccc(-c2ccc(N(c3ccccc3)c3ccc4c(c3)oc3cccc(-c5cccc(-c6nc(-c7ccccc7)nc(-c7ccccc7)n6)c5)c34)cc2)cc1 ; c1ccc(-c2nc(-c3ccccc3)nc(-c3ccc(-c4cccc5oc6cc(N(c7ccccc7)c7ccccc7)ccc6c45)c4ccccc34)n2)cc1 ; c1ccc(-c2nc(-c3ccccc3)nc(-c3ccc(-c4cccc5oc6cc(N(c7ccccc7)c7ccc8ccccc8c7)ccc6c45)cc3)n2)cc1]

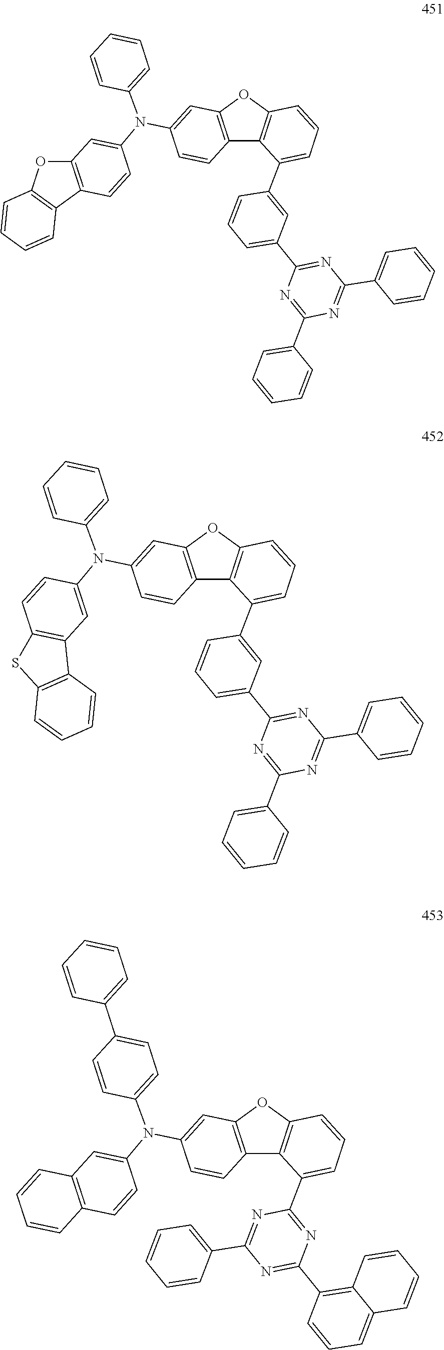 [SMILES: c1ccc(-c2nc(-c3ccccc3)nc(-c3cccc(-c4cccc5oc6cc(N(c7ccccc7)c7ccc8c(c7)oc7ccccc78)ccc6c45)c3)n2)cc1 ; c1ccc(-c2nc(-c3ccccc3)nc(-c3cccc(-c4cccc5oc6cc(N(c7ccccc7)c7ccc8sc9ccccc9c8c7)ccc6c45)c3)n2)cc1 ; c1ccc(-c2ccc(N(c3ccc4ccccc4c3)c3ccc4c(c3)oc3cccc(-c5nc(-c6ccccc6)nc(-c6cccc7ccccc67)n5)c34)cc2)cc1]

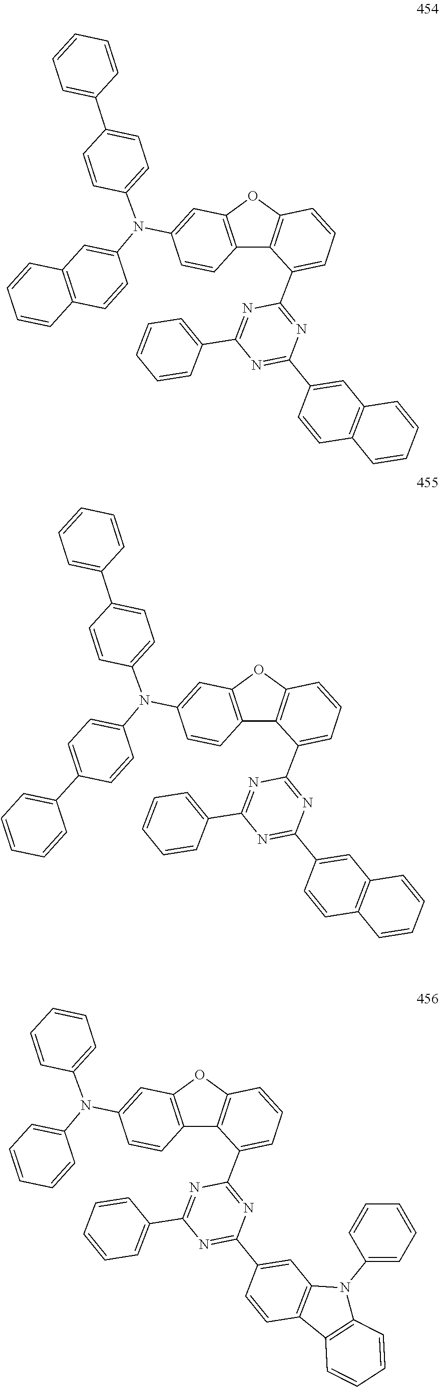 [SMILES: c1ccc(-c2ccc(N(c3ccc4ccccc4c3)c3ccc4c(c3)oc3cccc(-c5nc(-c6ccccc6)nc(-c6ccc7ccccc7c6)n5)c34)cc2)cc1 ; c1ccc(-c2ccc(N(c3ccc(-c4ccccc4)cc3)c3ccc4c(c3)oc3cccc(-c5nc(-c6ccccc6)nc(-c6ccc7ccccc7c6)n5)c34)cc2)cc1 ; c1ccc(-c2nc(-c3ccc4c5ccccc5n(-c5ccccc5)c4c3)nc(-c3cccc4oc5cc(N(c6ccccc6)c6ccccc6)ccc5c34)n2)cc1]

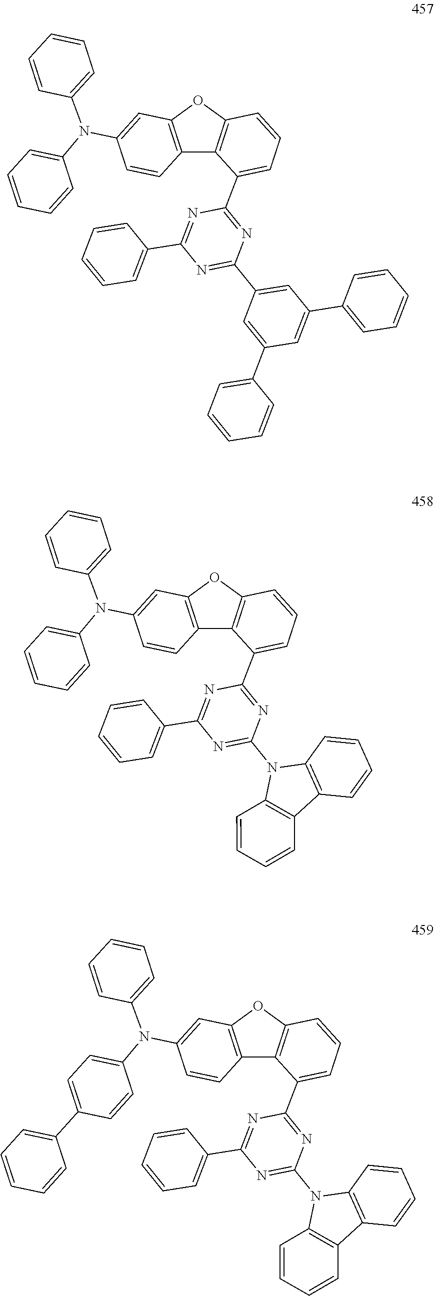 [SMILES: c1ccc(-c2cc(-c3ccccc3)cc(-c3nc(-c4ccccc4)nc(-c4cccc5oc6cc(N(c7ccccc7)c7ccccc7)ccc6c45)n3)c2)cc1 ; c1ccc(-c2nc(-c3cccc4oc5cc(N(c6ccccc6)c6ccccc6)ccc5c34)nc(-n3c4ccccc4c4ccccc43)n2)cc1 ; c1ccc(-c2ccc(N(c3ccccc3)c3ccc4c(c3)oc3cccc(-c5nc(-c6ccccc6)nc(-n6c7ccccc7c7ccccc76)n5)c34)cc2)cc1]

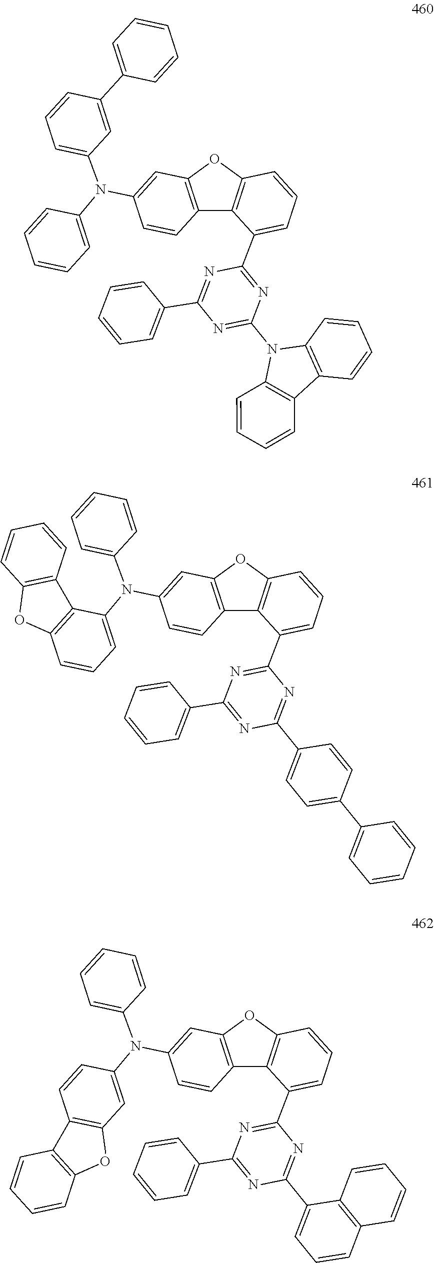 [SMILES: c1ccc(-c2cccc(N(c3ccccc3)c3ccc4c(c3)oc3cccc(-c5nc(-c6ccccc6)nc(-n6c7ccccc7c7ccccc76)n5)c34)c2)cc1 ; c1ccc(-c2ccc(-c3nc(-c4ccccc4)nc(-c4cccc5oc6cc(N(c7ccccc7)c7cccc8oc9ccccc9c78)ccc6c45)n3)cc2)cc1 ; c1ccc(-c2nc(-c3cccc4ccccc34)nc(-c3cccc4oc5cc(N(c6ccccc6)c6ccc7c(c6)oc6ccccc67)ccc5c34)n2)cc1]

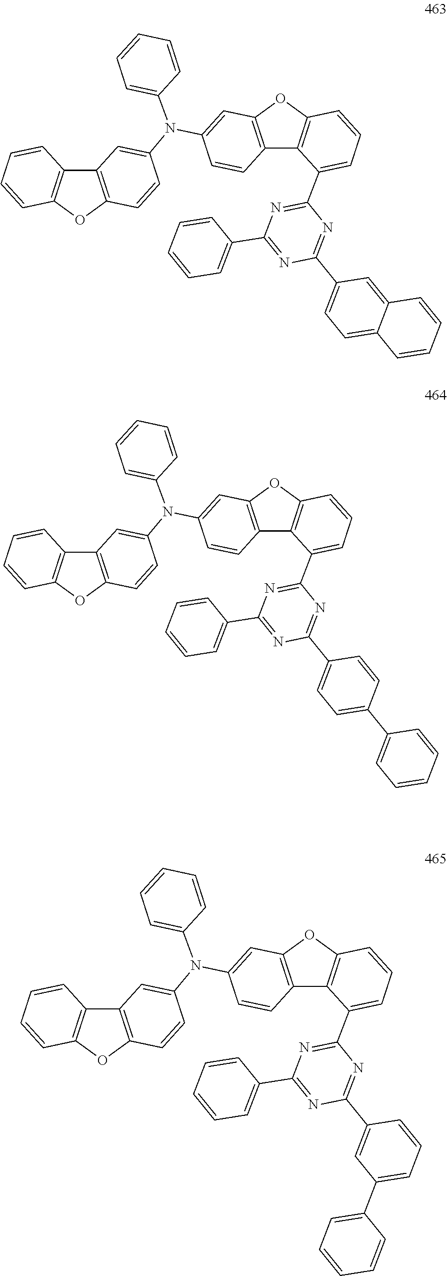 [SMILES: c1ccc(-c2nc(-c3ccc4ccccc4c3)nc(-c3cccc4oc5cc(N(c6ccccc6)c6ccc7oc8ccccc8c7c6)ccc5c34)n2)cc1 ; c1ccc(-c2ccc(-c3nc(-c4ccccc4)nc(-c4cccc5oc6cc(N(c7ccccc7)c7ccc8oc9ccccc9c8c7)ccc6c45)n3)cc2)cc1 ; c1ccc(-c2cccc(-c3nc(-c4ccccc4)nc(-c4cccc5oc6cc(N(c7ccccc7)c7ccc8oc9ccccc9c8c7)ccc6c45)n3)c2)cc1]

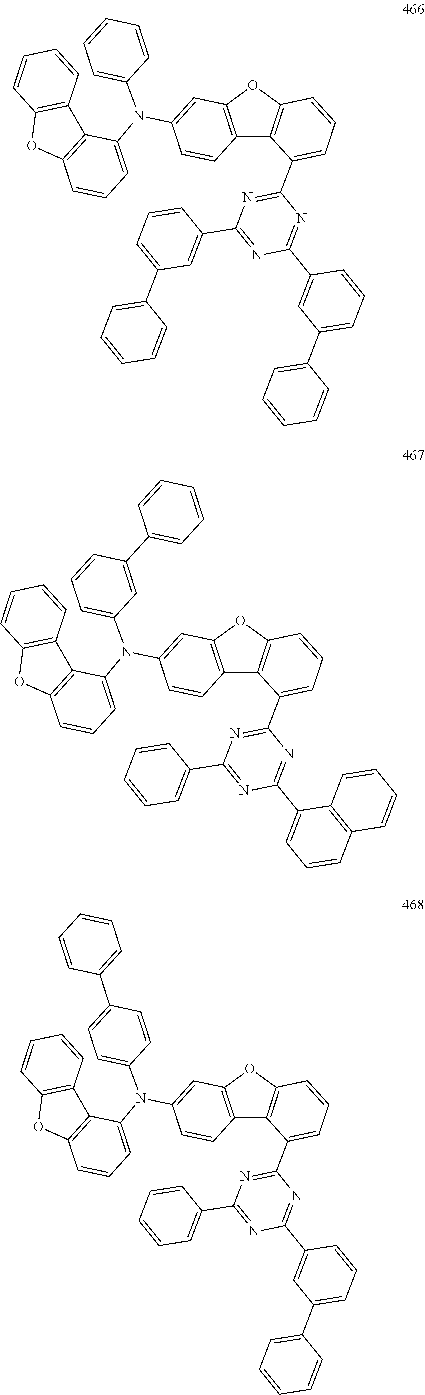 [SMILES: c1ccc(-c2cccc(-c3nc(-c4cccc(-c5ccccc5)c4)nc(-c4cccc5oc6cc(N(c7ccccc7)c7cccc8oc9ccccc9c78)ccc6c45)n3)c2)cc1 ; c1ccc(-c2cccc(N(c3ccc4c(c3)oc3cccc(-c5nc(-c6ccccc6)nc(-c6cccc7ccccc67)n5)c34)c3cccc4oc5ccccc5c34)c2)cc1 ; c1ccc(-c2ccc(N(c3ccc4c(c3)oc3cccc(-c5nc(-c6ccccc6)nc(-c6cccc(-c7ccccc7)c6)n5)c34)c3cccc4oc5ccccc5c34)cc2)cc1]

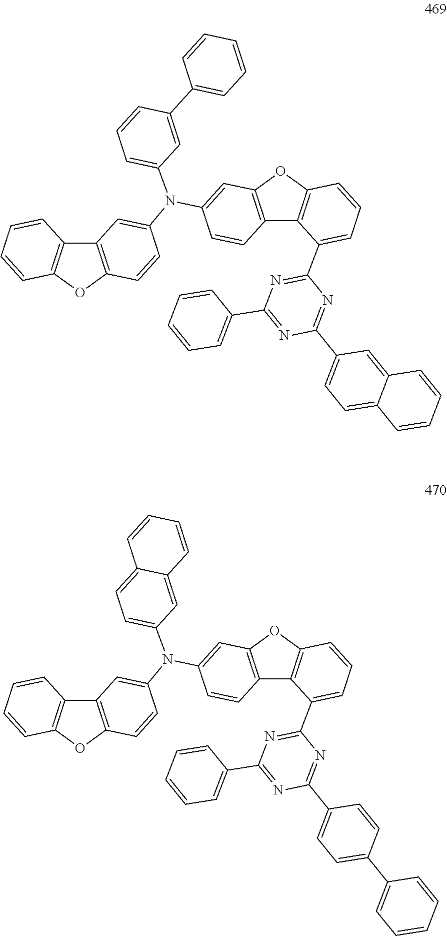 [SMILES: c1ccc(-c2cccc(N(c3ccc4c(c3)oc3cccc(-c5nc(-c6ccccc6)nc(-c6ccc7ccccc7c6)n5)c34)c3ccc4oc5ccccc5c4c3)c2)cc1 ; c1ccc(-c2ccc(-c3nc(-c4ccccc4)nc(-c4cccc5oc6cc(N(c7ccc8ccccc8c7)c7ccc8oc9ccccc9c8c7)ccc6c45)n3)cc2)cc1]

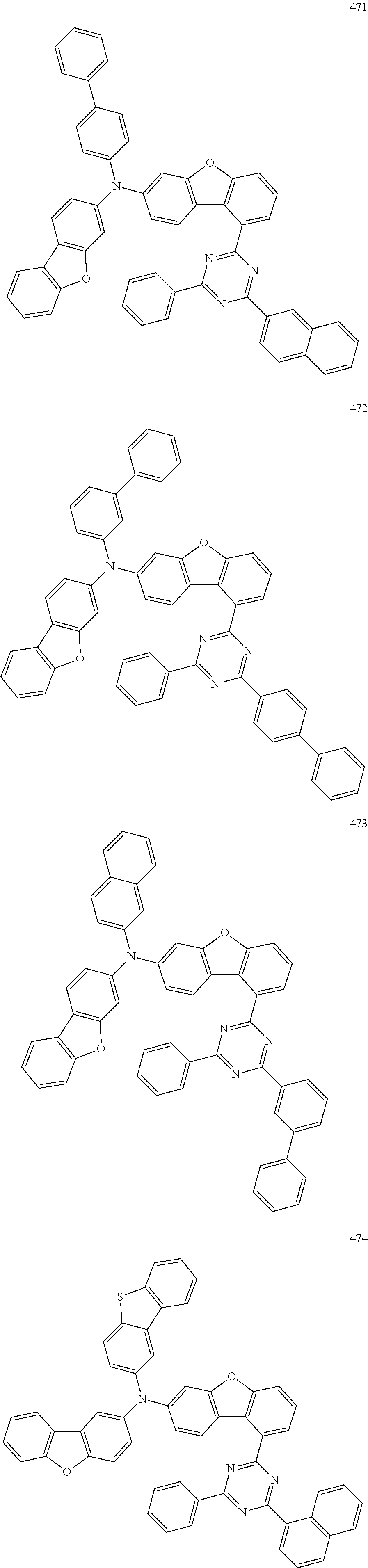 [SMILES: c1ccc(-c2ccc(N(c3ccc4c(c3)oc3ccccc34)c3ccc4c(c3)oc3cccc(-c5nc(-c6ccccc6)nc(-c6ccc7ccccc7c6)n5)c34)cc2)cc1 ; c1ccc(-c2ccc(-c3nc(-c4ccccc4)nc(-c4cccc5oc6cc(N(c7cccc(-c8ccccc8)c7)c7ccc8c(c7)oc7ccccc78)ccc6c45)n3)cc2)cc1 ; c1ccc(-c2cccc(-c3nc(-c4ccccc4)nc(-c4cccc5oc6cc(N(c7ccc8ccccc8c7)c7ccc8c(c7)oc7ccccc78)ccc6c45)n3)c2)cc1 ; c1ccc(-c2nc(-c3cccc4ccccc34)nc(-c3cccc4oc5cc(N(c6ccc7oc8ccccc8c7c6)c6ccc7sc8ccccc8c7c6)ccc5c34)n2)cc1]

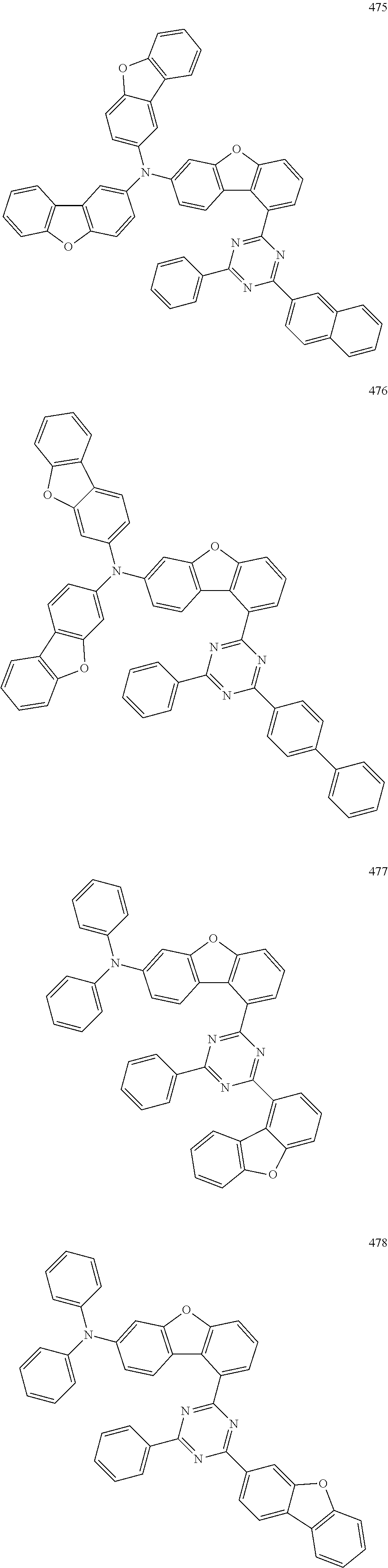 [SMILES: c1ccc(-c2nc(-c3ccc4ccccc4c3)nc(-c3cccc4oc5cc(N(c6ccc7oc8ccccc8c7c6)c6ccc7oc8ccccc8c7c6)ccc5c34)n2)cc1 ; c1ccc(-c2ccc(-c3nc(-c4ccccc4)nc(-c4cccc5oc6cc(N(c7ccc8c(c7)oc7ccccc78)c7ccc8c(c7)oc7ccccc78)ccc6c45)n3)cc2)cc1 ; c1ccc(-c2nc(-c3cccc4oc5ccccc5c34)nc(-c3cccc4oc5cc(N(c6ccccc6)c6ccccc6)ccc5c34)n2)cc1 ; c1ccc(-c2nc(-c3ccc4c(c3)oc3ccccc34)nc(-c3cccc4oc5cc(N(c6ccccc6)c6ccccc6)ccc5c34)n2)cc1]

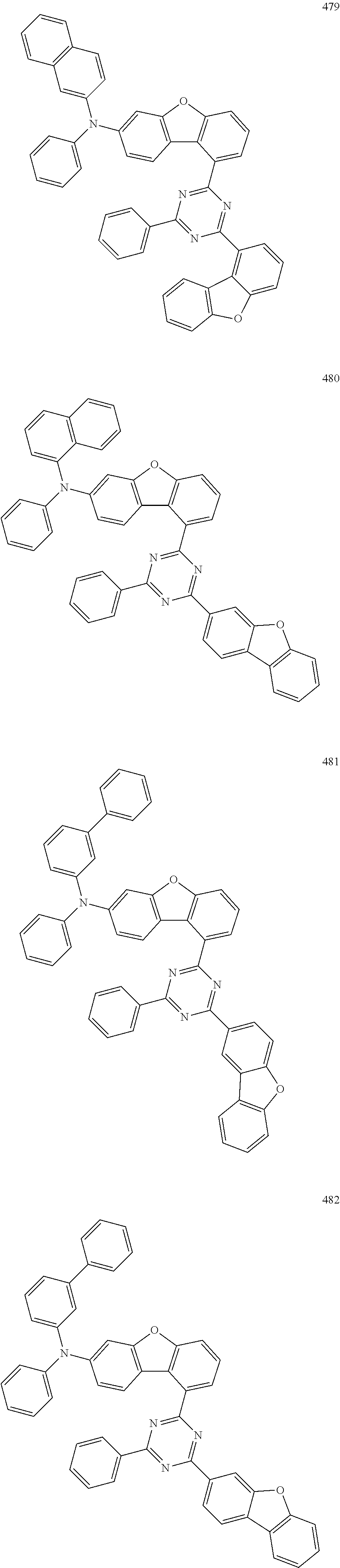 [SMILES: c1ccc(-c2nc(-c3cccc4oc5ccccc5c34)nc(-c3cccc4oc5cc(N(c6ccccc6)c6ccc7ccccc7c6)ccc5c34)n2)cc1 ; c1ccc(-c2nc(-c3ccc4c(c3)oc3ccccc34)nc(-c3cccc4oc5cc(N(c6ccccc6)c6cccc7ccccc67)ccc5c34)n2)cc1 ; c1ccc(-c2cccc(N(c3ccccc3)c3ccc4c(c3)oc3cccc(-c5nc(-c6ccccc6)nc(-c6ccc7oc8ccccc8c7c6)n5)c34)c2)cc1 ; c1ccc(-c2cccc(N(c3ccccc3)c3ccc4c(c3)oc3cccc(-c5nc(-c6ccccc6)nc(-c6ccc7c(c6)oc6ccccc67)n5)c34)c2)cc1]

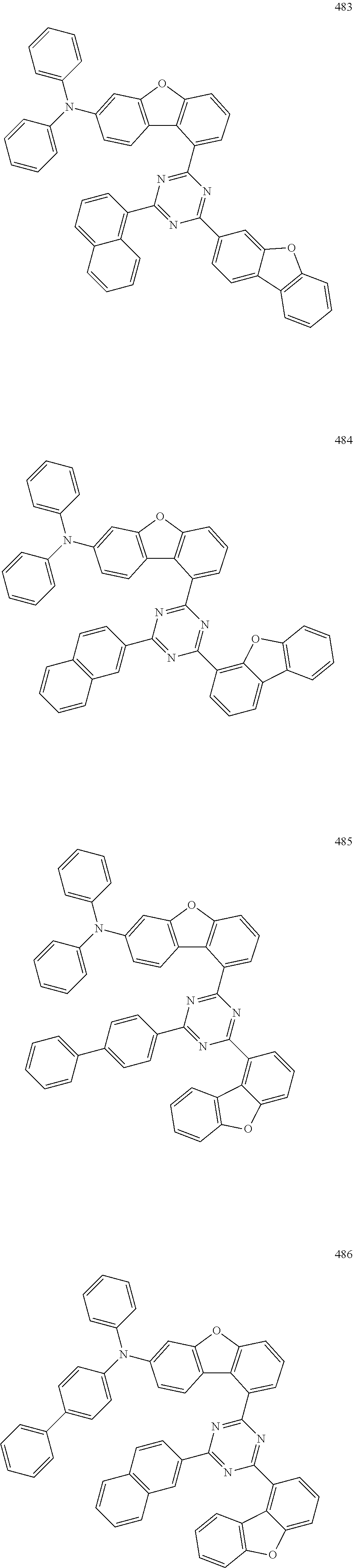 [SMILES: c1ccc(N(c2ccccc2)c2ccc3c(c2)oc2cccc(-c4nc(-c5ccc6c(c5)oc5ccccc56)nc(-c5cccc6ccccc56)n4)c23)cc1 ; c1ccc(N(c2ccccc2)c2ccc3c(c2)oc2cccc(-c4nc(-c5ccc6ccccc6c5)nc(-c5cccc6c5oc5ccccc56)n4)c23)cc1 ; c1ccc(-c2ccc(-c3nc(-c4cccc5oc6ccccc6c45)nc(-c4cccc5oc6cc(N(c7ccccc7)c7ccccc7)ccc6c45)n3)cc2)cc1 ; c1ccc(-c2ccc(N(c3ccccc3)c3ccc4c(c3)oc3cccc(-c5nc(-c6ccc7ccccc7c6)nc(-c6cccc7oc8ccccc8c67)n5)c34)cc2)cc1]

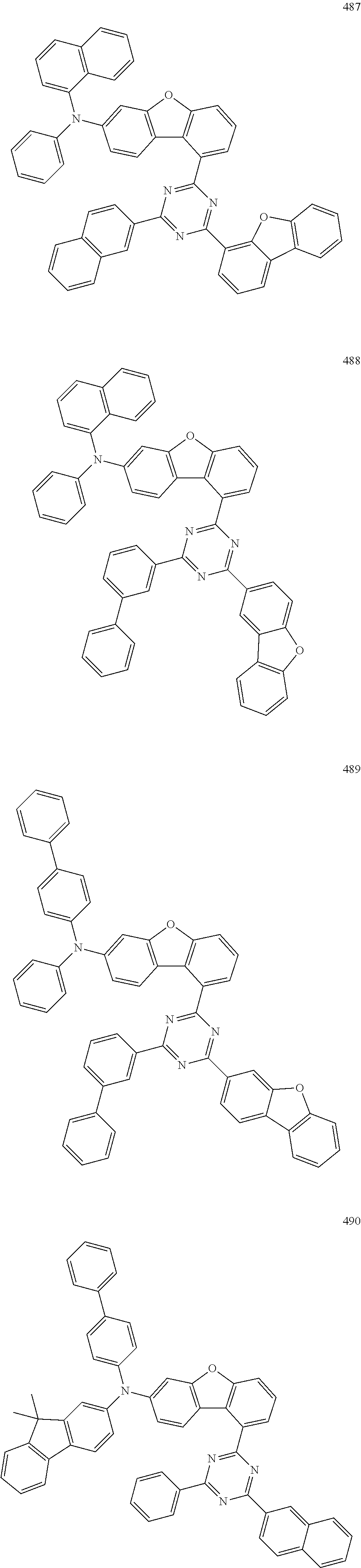 [SMILES: c1ccc(N(c2ccc3c(c2)oc2cccc(-c4nc(-c5ccc6ccccc6c5)nc(-c5cccc6c5oc5ccccc56)n4)c23)c2cccc3ccccc23)cc1 ; c1ccc(-c2cccc(-c3nc(-c4ccc5oc6ccccc6c5c4)nc(-c4cccc5oc6cc(N(c7ccccc7)c7cccc8ccccc78)ccc6c45)n3)c2)cc1 ; c1ccc(-c2ccc(N(c3ccccc3)c3ccc4c(c3)oc3cccc(-c5nc(-c6cccc(-c7ccccc7)c6)nc(-c6ccc7c(c6)oc6ccccc67)n5)c34)cc2)cc1 ; CC1(C)c2ccccc2-c2ccc(N(c3ccc(-c4ccccc4)cc3)c3ccc4c(c3)oc3cccc(-c5nc(-c6ccccc6)nc(-c6ccc7ccccc7c6)n5)c34)cc21]

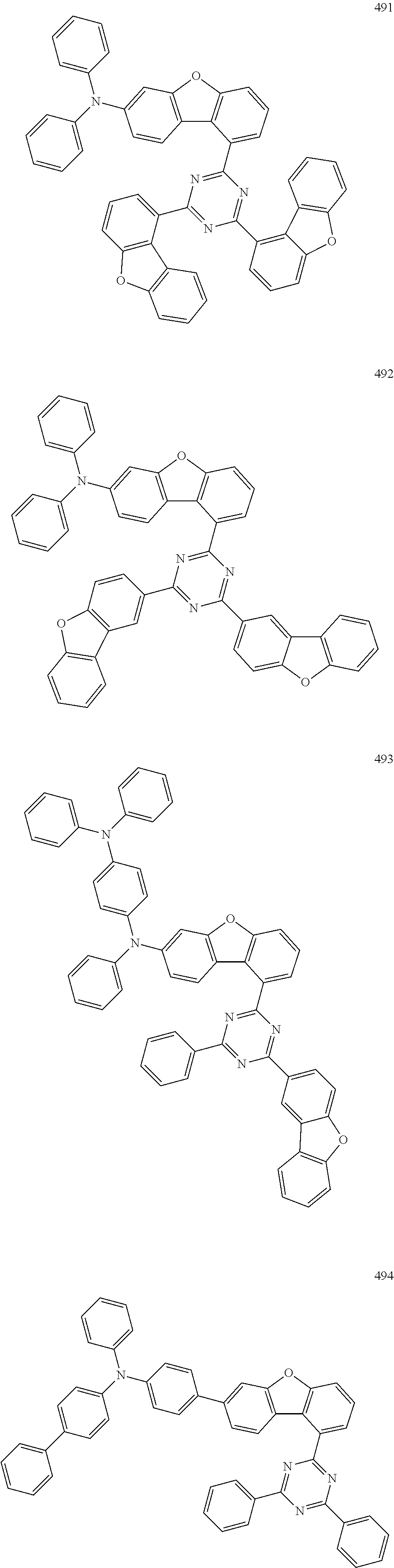 [SMILES: c1ccc(N(c2ccccc2)c2ccc3c(c2)oc2cccc(-c4nc(-c5cccc6oc7ccccc7c56)nc(-c5cccc6oc7ccccc7c56)n4)c23)cc1 ; c1ccc(N(c2ccccc2)c2ccc3c(c2)oc2cccc(-c4nc(-c5ccc6oc7ccccc7c6c5)nc(-c5ccc6oc7ccccc7c6c5)n4)c23)cc1 ; c1ccc(-c2nc(-c3ccc4oc5ccccc5c4c3)nc(-c3cccc4oc5cc(N(c6ccccc6)c6ccc(N(c7ccccc7)c7ccccc7)cc6)ccc5c34)n2)cc1 ; c1ccc(-c2ccc(N(c3ccccc3)c3ccc(-c4ccc5c(c4)oc4cccc(-c6nc(-c7ccccc7)nc(-c7ccccc7)n6)c45)cc3)cc2)cc1]

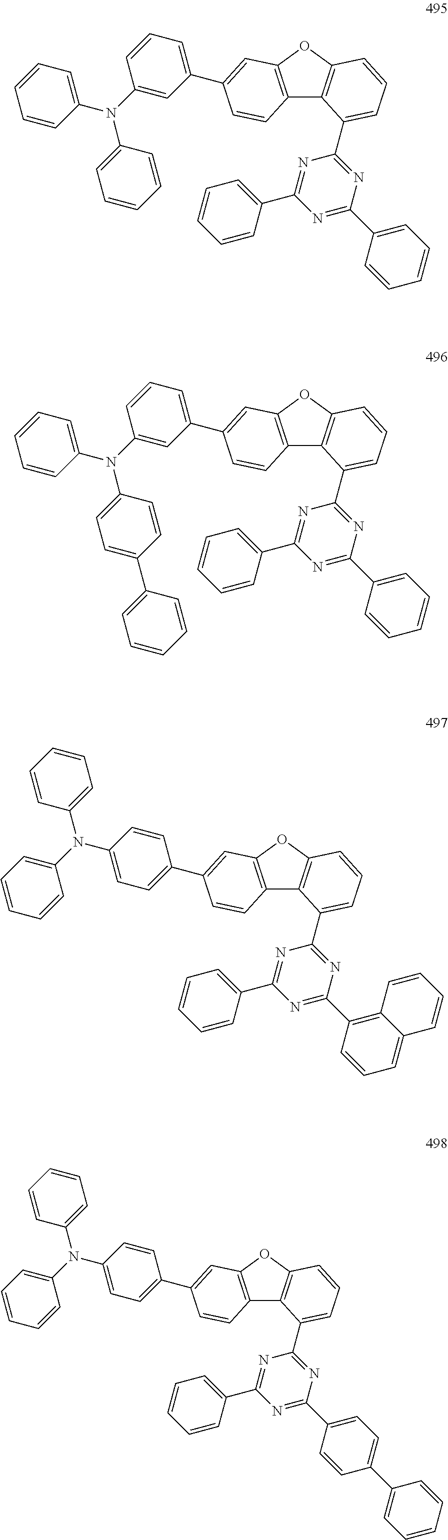 [SMILES: c1ccc(-c2nc(-c3ccccc3)nc(-c3cccc4oc5cc(-c6cccc(N(c7ccccc7)c7ccccc7)c6)ccc5c34)n2)cc1 ; c1ccc(-c2ccc(N(c3ccccc3)c3cccc(-c4ccc5c(c4)oc4cccc(-c6nc(-c7ccccc7)nc(-c7ccccc7)n6)c45)c3)cc2)cc1 ; c1ccc(-c2nc(-c3cccc4ccccc34)nc(-c3cccc4oc5cc(-c6ccc(N(c7ccccc7)c7ccccc7)cc6)ccc5c34)n2)cc1 ; c1ccc(-c2ccc(-c3nc(-c4ccccc4)nc(-c4cccc5oc6cc(-c7ccc(N(c8ccccc8)c8ccccc8)cc7)ccc6c45)n3)cc2)cc1]

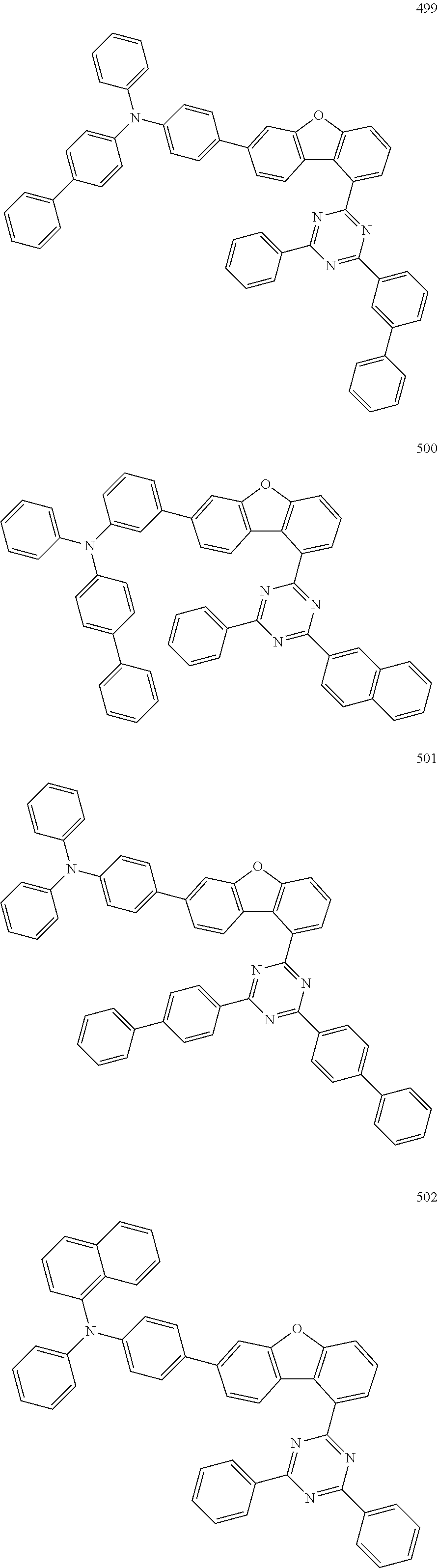 [SMILES: c1ccc(-c2ccc(N(c3ccccc3)c3ccc(-c4ccc5c(c4)oc4cccc(-c6nc(-c7ccccc7)nc(-c7cccc(-c8ccccc8)c7)n6)c45)cc3)cc2)cc1 ; c1ccc(-c2ccc(N(c3ccccc3)c3cccc(-c4ccc5c(c4)oc4cccc(-c6nc(-c7ccccc7)nc(-c7ccc8ccccc8c7)n6)c45)c3)cc2)cc1 ; c1ccc(-c2ccc(-c3nc(-c4ccc(-c5ccccc5)cc4)nc(-c4cccc5oc6cc(-c7ccc(N(c8ccccc8)c8ccccc8)cc7)ccc6c45)n3)cc2)cc1 ; c1ccc(-c2nc(-c3ccccc3)nc(-c3cccc4oc5cc(-c6ccc(N(c7ccccc7)c7cccc8ccccc78)cc6)ccc5c34)n2)cc1]

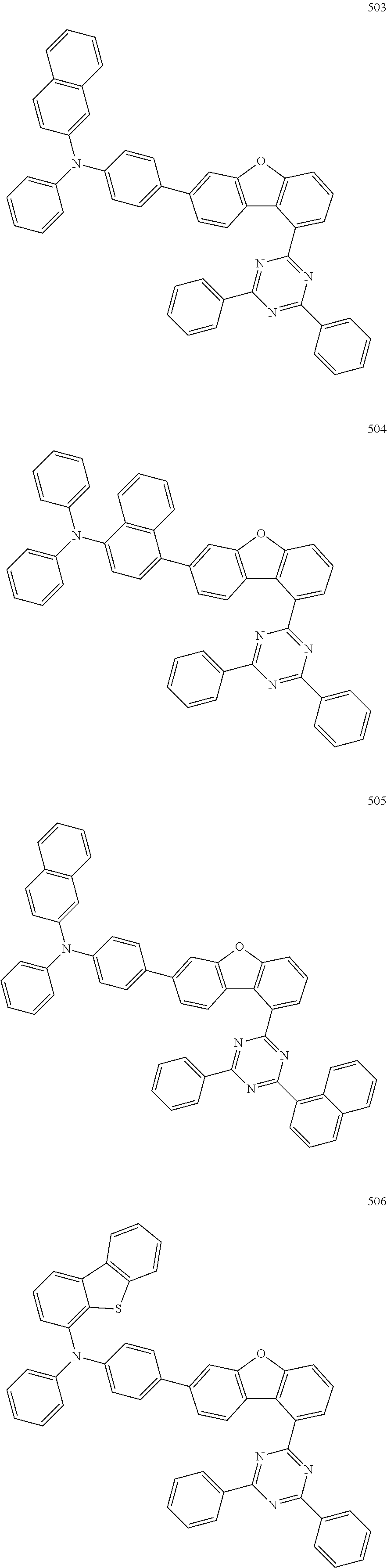 [SMILES: c1ccc(-c2nc(-c3ccccc3)nc(-c3cccc4oc5cc(-c6ccc(N(c7ccccc7)c7ccc8ccccc8c7)cc6)ccc5c34)n2)cc1 ; c1ccc(-c2nc(-c3ccccc3)nc(-c3cccc4oc5cc(-c6ccc(N(c7ccccc7)c7ccccc7)c7ccccc67)ccc5c34)n2)cc1 ; c1ccc(-c2nc(-c3cccc4ccccc34)nc(-c3cccc4oc5cc(-c6ccc(N(c7ccccc7)c7ccc8ccccc8c7)cc6)ccc5c34)n2)cc1 ; c1ccc(-c2nc(-c3ccccc3)nc(-c3cccc4oc5cc(-c6ccc(N(c7ccccc7)c7cccc8c7sc7ccccc78)cc6)ccc5c34)n2)cc1]

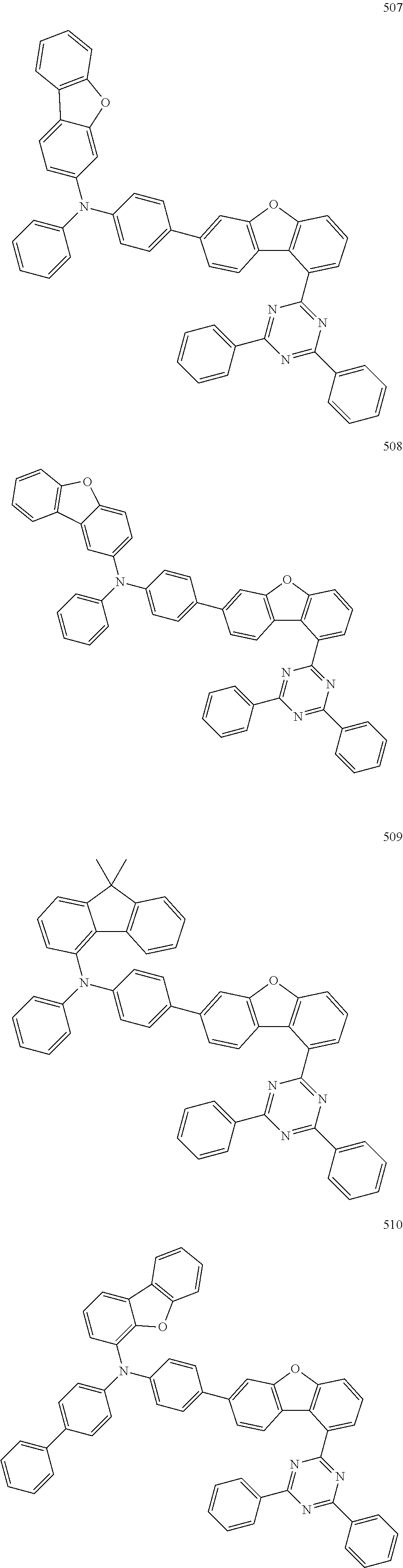 [SMILES: c1ccc(-c2nc(-c3ccccc3)nc(-c3cccc4oc5cc(-c6ccc(N(c7ccccc7)c7ccc8c(c7)oc7ccccc78)cc6)ccc5c34)n2)cc1 ; c1ccc(-c2nc(-c3ccccc3)nc(-c3cccc4oc5cc(-c6ccc(N(c7ccccc7)c7ccc8oc9ccccc9c8c7)cc6)ccc5c34)n2)cc1 ; CC1(C)c2ccccc2-c2c(N(c3ccccc3)c3ccc(-c4ccc5c(c4)oc4cccc(-c6nc(-c7ccccc7)nc(-c7ccccc7)n6)c45)cc3)cccc21 ; c1ccc(-c2ccc(N(c3ccc(-c4ccc5c(c4)oc4cccc(-c6nc(-c7ccccc7)nc(-c7ccccc7)n6)c45)cc3)c3cccc4c3oc3ccccc34)cc2)cc1]

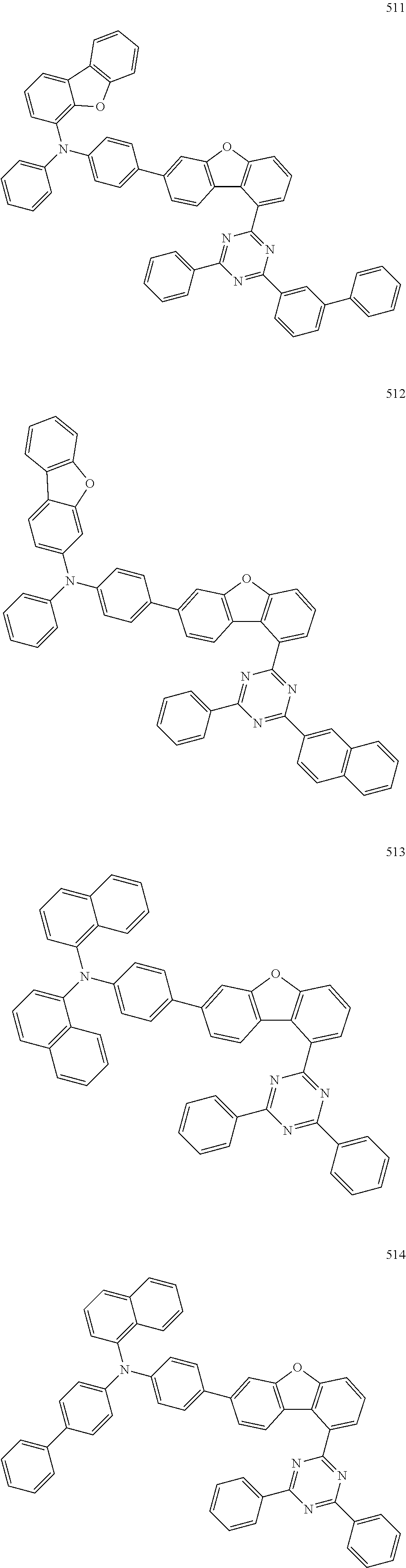 [SMILES: c1ccc(-c2cccc(-c3nc(-c4ccccc4)nc(-c4cccc5oc6cc(-c7ccc(N(c8ccccc8)c8cccc9c8oc8ccccc89)cc7)ccc6c45)n3)c2)cc1 ; c1ccc(-c2nc(-c3ccc4ccccc4c3)nc(-c3cccc4oc5cc(-c6ccc(N(c7ccccc7)c7ccc8c(c7)oc7ccccc78)cc6)ccc5c34)n2)cc1 ; c1ccc(-c2nc(-c3ccccc3)nc(-c3cccc4oc5cc(-c6ccc(N(c7cccc8ccccc78)c7cccc8ccccc78)cc6)ccc5c34)n2)cc1 ; c1ccc(-c2ccc(N(c3ccc(-c4ccc5c(c4)oc4cccc(-c6nc(-c7ccccc7)nc(-c7ccccc7)n6)c45)cc3)c3cccc4ccccc34)cc2)cc1]

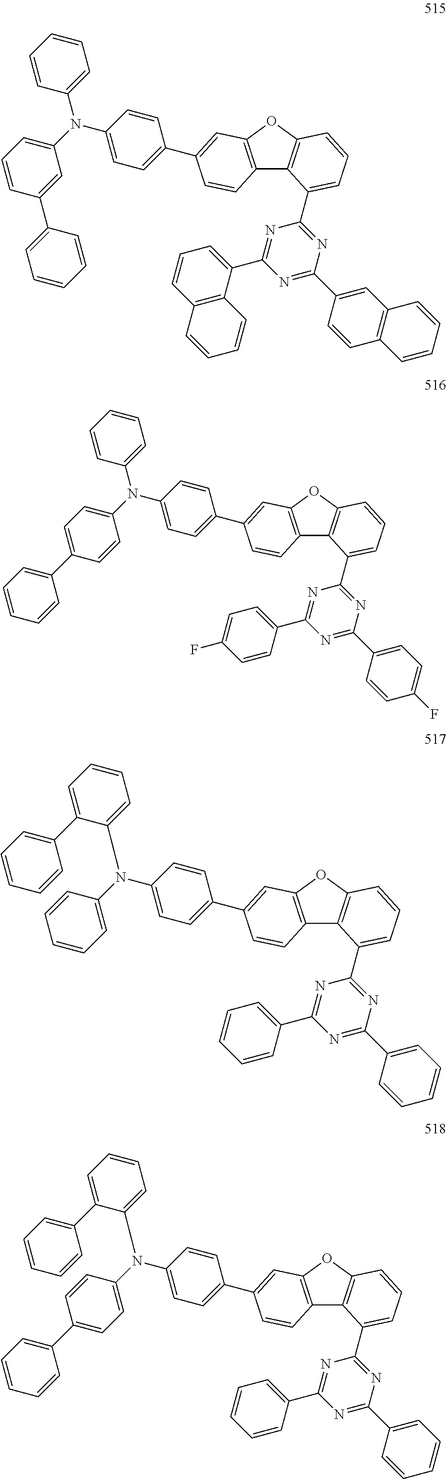 [SMILES: c1ccc(-c2cccc(N(c3ccccc3)c3ccc(-c4ccc5c(c4)oc4cccc(-c6nc(-c7ccc8ccccc8c7)nc(-c7cccc8ccccc78)n6)c45)cc3)c2)cc1 ; Fc1ccc(-c2nc(-c3ccc(F)cc3)nc(-c3cccc4oc5cc(-c6ccc(N(c7ccccc7)c7ccc(-c8ccccc8)cc7)cc6)ccc5c34)n2)cc1 ; c1ccc(-c2nc(-c3ccccc3)nc(-c3cccc4oc5cc(-c6ccc(N(c7ccccc7)c7ccccc7-c7ccccc7)cc6)ccc5c34)n2)cc1 ; c1ccc(-c2ccc(N(c3ccc(-c4ccc5c(c4)oc4cccc(-c6nc(-c7ccccc7)nc(-c7ccccc7)n6)c45)cc3)c3ccccc3-c3ccccc3)cc2)cc1]

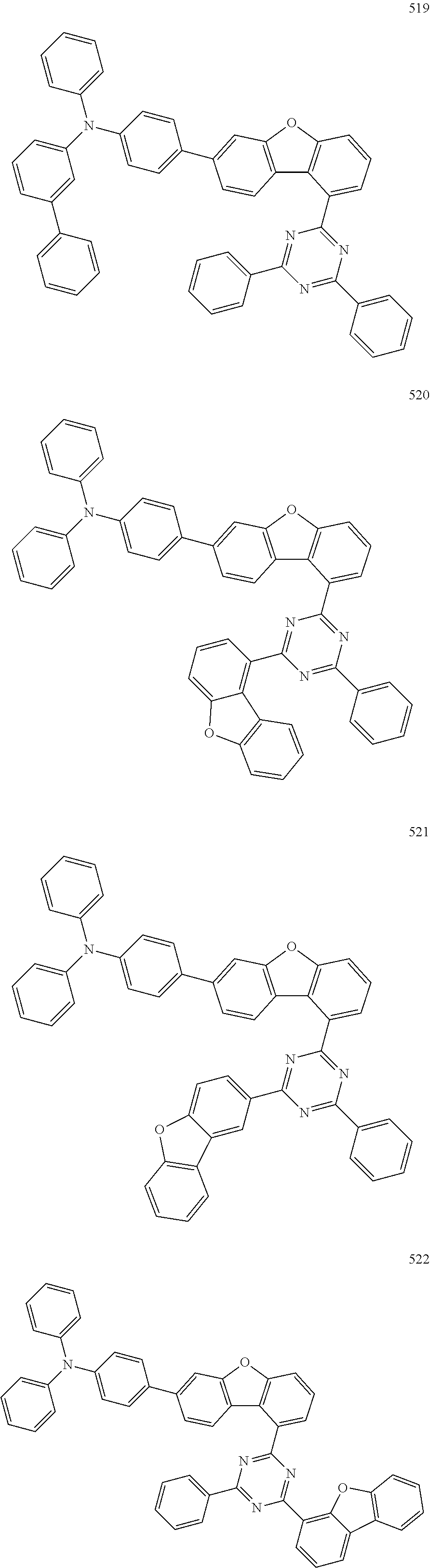 [SMILES: c1ccc(-c2cccc(N(c3ccccc3)c3ccc(-c4ccc5c(c4)oc4cccc(-c6nc(-c7ccccc7)nc(-c7ccccc7)n6)c45)cc3)c2)cc1 ; c1ccc(-c2nc(-c3cccc4oc5ccccc5c34)nc(-c3cccc4oc5cc(-c6ccc(N(c7ccccc7)c7ccccc7)cc6)ccc5c34)n2)cc1 ; c1ccc(-c2nc(-c3ccc4oc5ccccc5c4c3)nc(-c3cccc4oc5cc(-c6ccc(N(c7ccccc7)c7ccccc7)cc6)ccc5c34)n2)cc1 ; c1ccc(-c2nc(-c3cccc4c3oc3ccccc34)nc(-c3cccc4oc5cc(-c6ccc(N(c7ccccc7)c7ccccc7)cc6)ccc5c34)n2)cc1]

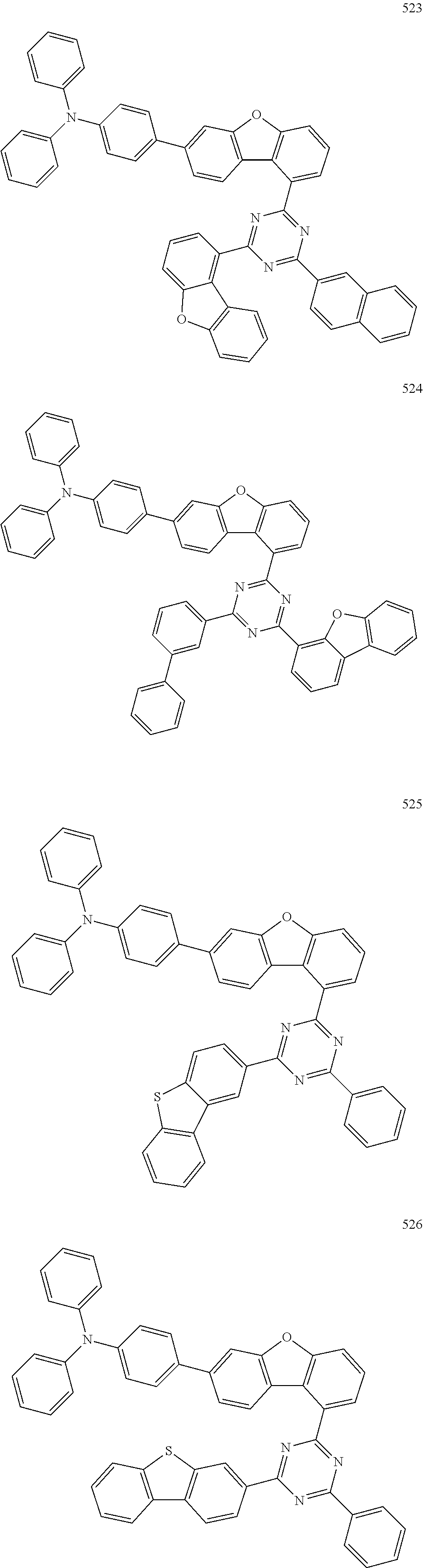 [SMILES: c1ccc(N(c2ccccc2)c2ccc(-c3ccc4c(c3)oc3cccc(-c5nc(-c6ccc7ccccc7c6)nc(-c6cccc7oc8ccccc8c67)n5)c34)cc2)cc1 ; c1ccc(-c2cccc(-c3nc(-c4cccc5c4oc4ccccc45)nc(-c4cccc5oc6cc(-c7ccc(N(c8ccccc8)c8ccccc8)cc7)ccc6c45)n3)c2)cc1 ; c1ccc(-c2nc(-c3ccc4sc5ccccc5c4c3)nc(-c3cccc4oc5cc(-c6ccc(N(c7ccccc7)c7ccccc7)cc6)ccc5c34)n2)cc1 ; c1ccc(-c2nc(-c3ccc4c(c3)sc3ccccc34)nc(-c3cccc4oc5cc(-c6ccc(N(c7ccccc7)c7ccccc7)cc6)ccc5c34)n2)cc1]

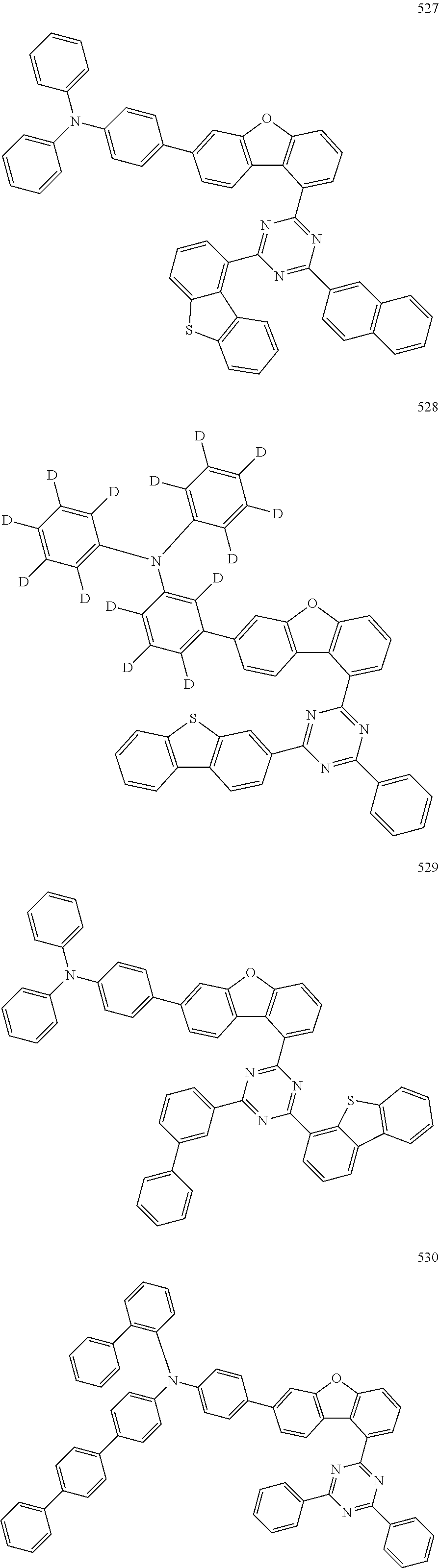

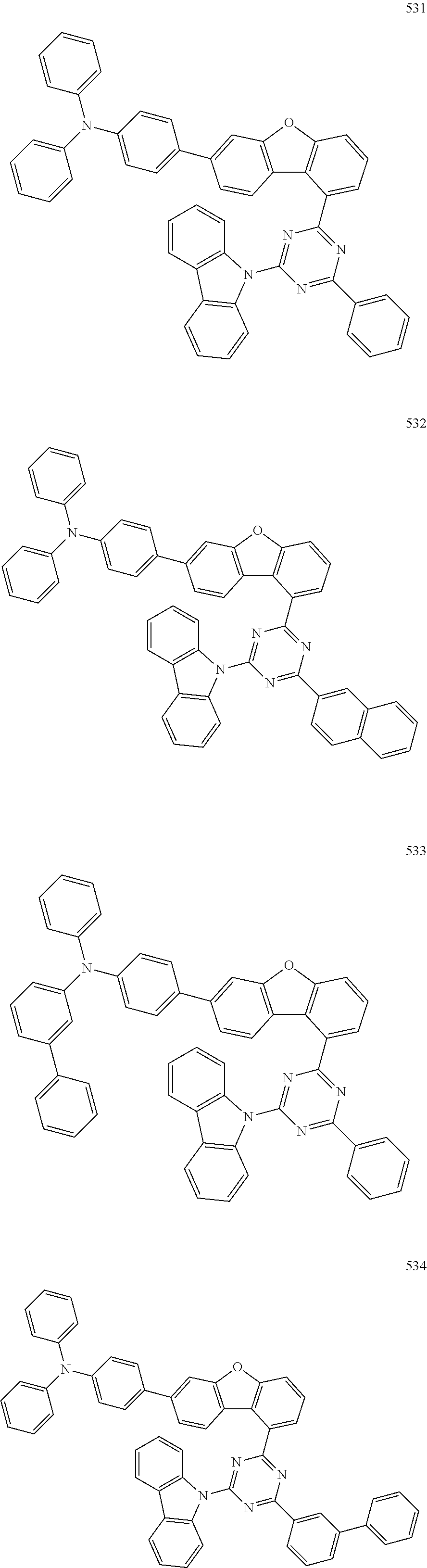 [SMILES: c1ccc(-c2nc(-c3cccc4oc5cc(-c6ccc(N(c7ccccc7)c7ccccc7)cc6)ccc5c34)nc(-n3c4ccccc4c4ccccc43)n2)cc1 ; c1ccc(N(c2ccccc2)c2ccc(-c3ccc4c(c3)oc3cccc(-c5nc(-c6ccc7ccccc7c6)nc(-n6c7ccccc7c7ccccc76)n5)c34)cc2)cc1 ; c1ccc(-c2cccc(N(c3ccccc3)c3ccc(-c4ccc5c(c4)oc4cccc(-c6nc(-c7ccccc7)nc(-n7c8ccccc8c8ccccc87)n6)c45)cc3)c2)cc1 ; c1ccc(-c2cccc(-c3nc(-c4cccc5oc6cc(-c7ccc(N(c8ccccc8)c8ccccc8)cc7)ccc6c45)nc(-n4c5ccccc5c5ccccc54)n3)c2)cc1]

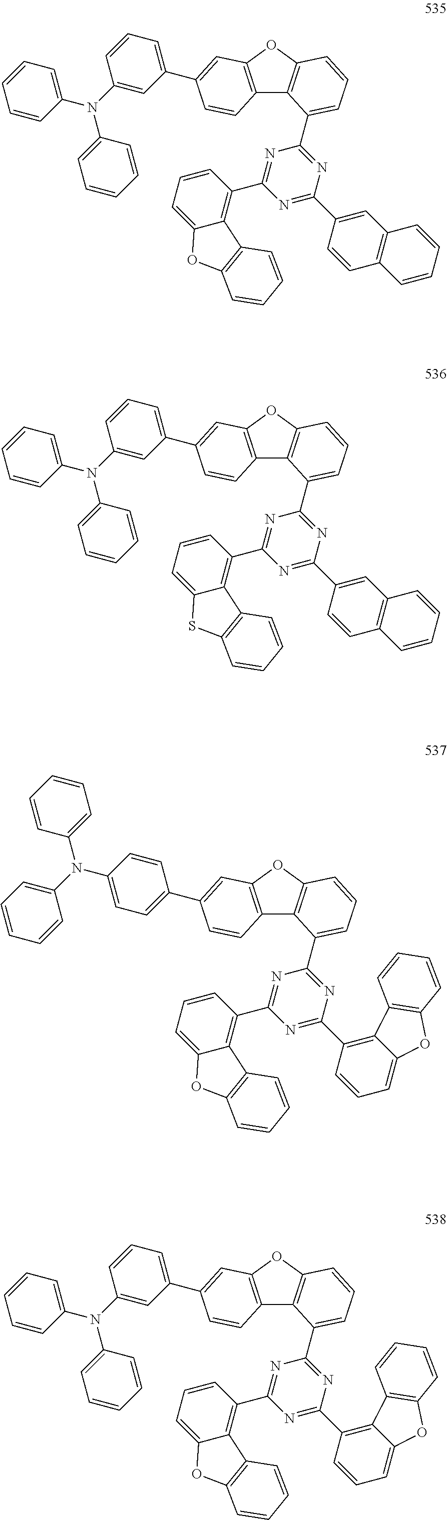 [SMILES: c1ccc(N(c2ccccc2)c2cccc(-c3ccc4c(c3)oc3cccc(-c5nc(-c6ccc7ccccc7c6)nc(-c6cccc7oc8ccccc8c67)n5)c34)c2)cc1 ; c1ccc(N(c2ccccc2)c2cccc(-c3ccc4c(c3)oc3cccc(-c5nc(-c6ccc7ccccc7c6)nc(-c6cccc7sc8ccccc8c67)n5)c34)c2)cc1 ; c1ccc(N(c2ccccc2)c2ccc(-c3ccc4c(c3)oc3cccc(-c5nc(-c6cccc7oc8ccccc8c67)nc(-c6cccc7oc8ccccc8c67)n5)c34)cc2)cc1 ; c1ccc(N(c2ccccc2)c2cccc(-c3ccc4c(c3)oc3cccc(-c5nc(-c6cccc7oc8ccccc8c67)nc(-c6cccc7oc8ccccc8c67)n5)c34)c2)cc1]

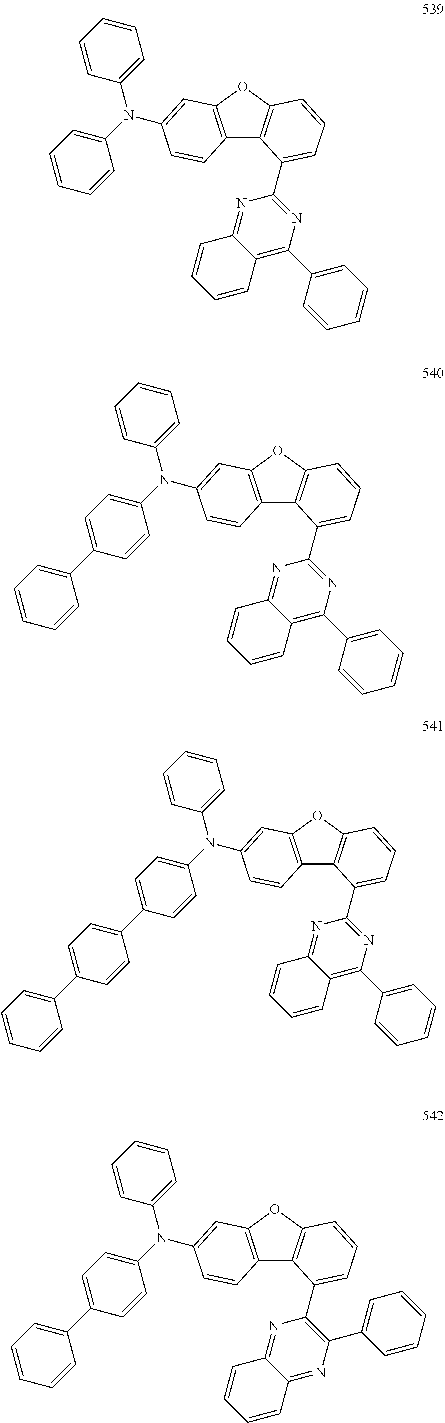 [SMILES: c1ccc(-c2nc(-c3cccc4oc5cc(N(c6ccccc6)c6ccccc6)ccc5c34)nc3ccccc23)cc1 ; c1ccc(-c2ccc(N(c3ccccc3)c3ccc4c(c3)oc3cccc(-c5nc(-c6ccccc6)c6ccccc6n5)c34)cc2)cc1 ; c1ccc(-c2ccc(-c3ccc(N(c4ccccc4)c4ccc5c(c4)oc4cccc(-c6nc(-c7ccccc7)c7ccccc7n6)c45)cc3)cc2)cc1 ; c1ccc(-c2ccc(N(c3ccccc3)c3ccc4c(c3)oc3cccc(-c5nc6ccccc6nc5-c5ccccc5)c34)cc2)cc1]

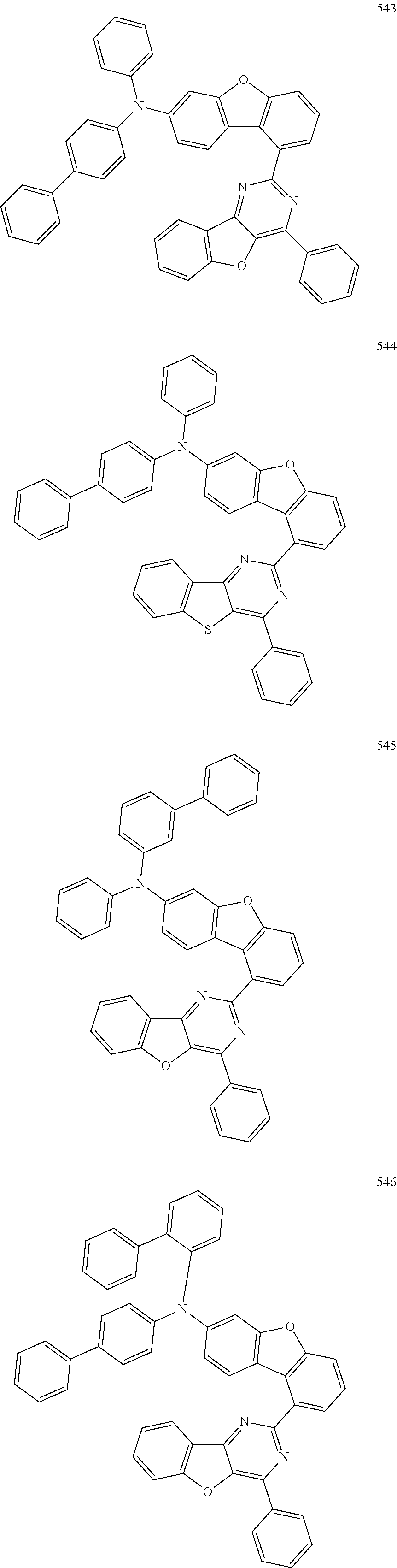 [SMILES: c1ccc(-c2ccc(N(c3ccccc3)c3ccc4c(c3)oc3cccc(-c5nc(-c6ccccc6)c6oc7ccccc7c6n5)c34)cc2)cc1 ; c1ccc(-c2ccc(N(c3ccccc3)c3ccc4c(c3)oc3cccc(-c5nc(-c6ccccc6)c6sc7ccccc7c6n5)c34)cc2)cc1 ; c1ccc(-c2cccc(N(c3ccccc3)c3ccc4c(c3)oc3cccc(-c5nc(-c6ccccc6)c6oc7ccccc7c6n5)c34)c2)cc1 ; c1ccc(-c2ccc(N(c3ccc4c(c3)oc3cccc(-c5nc(-c6ccccc6)c6oc7ccccc7c6n5)c34)c3ccccc3-c3ccccc3)cc2)cc1]

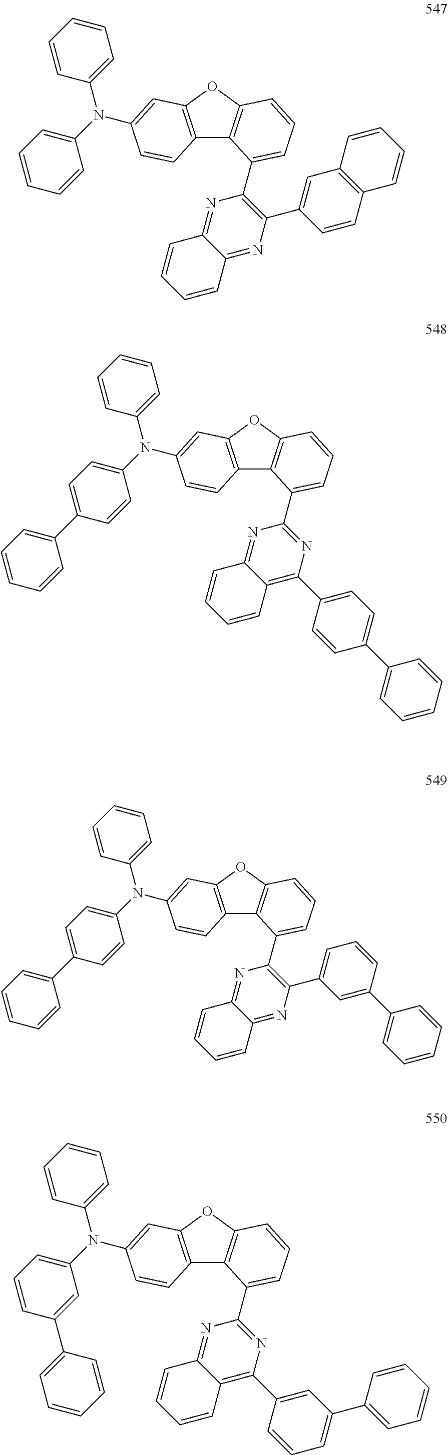 [SMILES: c1ccc(N(c2ccccc2)c2ccc3c(c2)oc2cccc(-c4nc5ccccc5nc4-c4ccc5ccccc5c4)c23)cc1 ; c1ccc(-c2ccc(-c3nc(-c4cccc5oc6cc(N(c7ccccc7)c7ccc(-c8ccccc8)cc7)ccc6c45)nc4ccccc34)cc2)cc1 ; c1ccc(-c2ccc(N(c3ccccc3)c3ccc4c(c3)oc3cccc(-c5nc6ccccc6nc5-c5cccc(-c6ccccc6)c5)c34)cc2)cc1 ; c1ccc(-c2cccc(-c3nc(-c4cccc5oc6cc(N(c7ccccc7)c7cccc(-c8ccccc8)c7)ccc6c45)nc4ccccc34)c2)cc1]

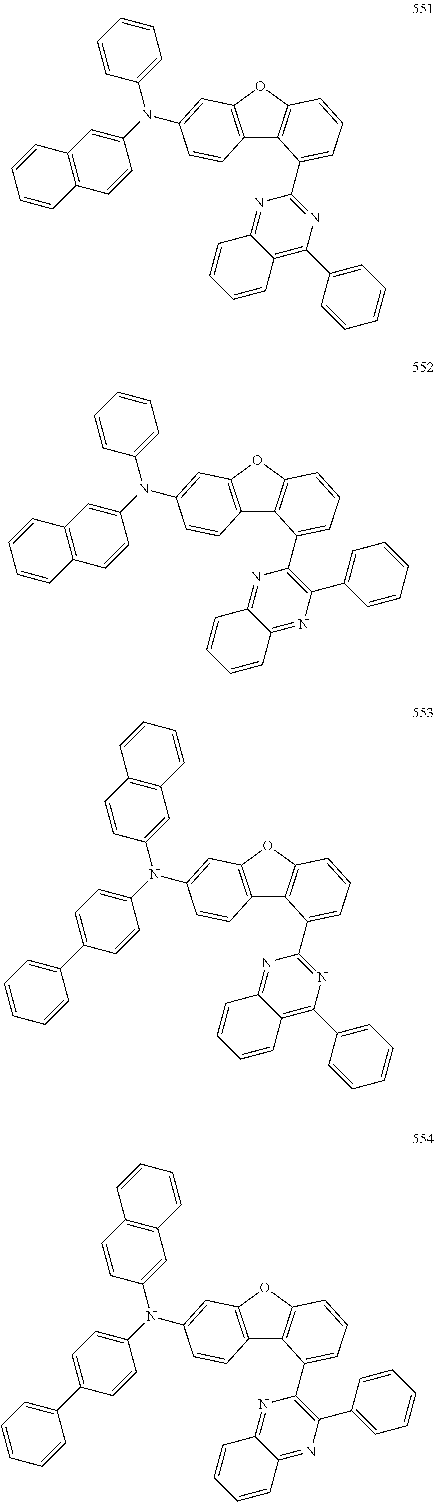 [SMILES: c1ccc(-c2nc(-c3cccc4oc5cc(N(c6ccccc6)c6ccc7ccccc7c6)ccc5c34)nc3ccccc23)cc1 ; c1ccc(-c2nc3ccccc3nc2-c2cccc3oc4cc(N(c5ccccc5)c5ccc6ccccc6c5)ccc4c23)cc1 ; c1ccc(-c2ccc(N(c3ccc4ccccc4c3)c3ccc4c(c3)oc3cccc(-c5nc(-c6ccccc6)c6ccccc6n5)c34)cc2)cc1 ; c1ccc(-c2ccc(N(c3ccc4ccccc4c3)c3ccc4c(c3)oc3cccc(-c5nc6ccccc6nc5-c5ccccc5)c34)cc2)cc1]

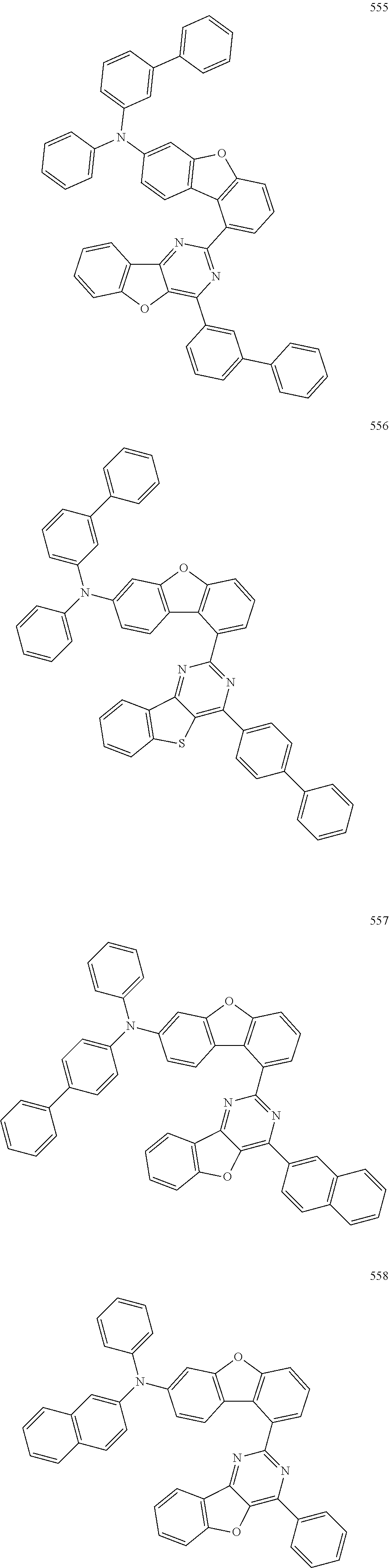 [SMILES: c1ccc(-c2cccc(-c3nc(-c4cccc5oc6cc(N(c7ccccc7)c7cccc(-c8ccccc8)c7)ccc6c45)nc4c3oc3ccccc34)c2)cc1 ; c1ccc(-c2ccc(-c3nc(-c4cccc5oc6cc(N(c7ccccc7)c7cccc(-c8ccccc8)c7)ccc6c45)nc4c3sc3ccccc34)cc2)cc1 ; c1ccc(-c2ccc(N(c3ccccc3)c3ccc4c(c3)oc3cccc(-c5nc(-c6ccc7ccccc7c6)c6oc7ccccc7c6n5)c34)cc2)cc1 ; c1ccc(-c2nc(-c3cccc4oc5cc(N(c6ccccc6)c6ccc7ccccc7c6)ccc5c34)nc3c2oc2ccccc23)cc1]

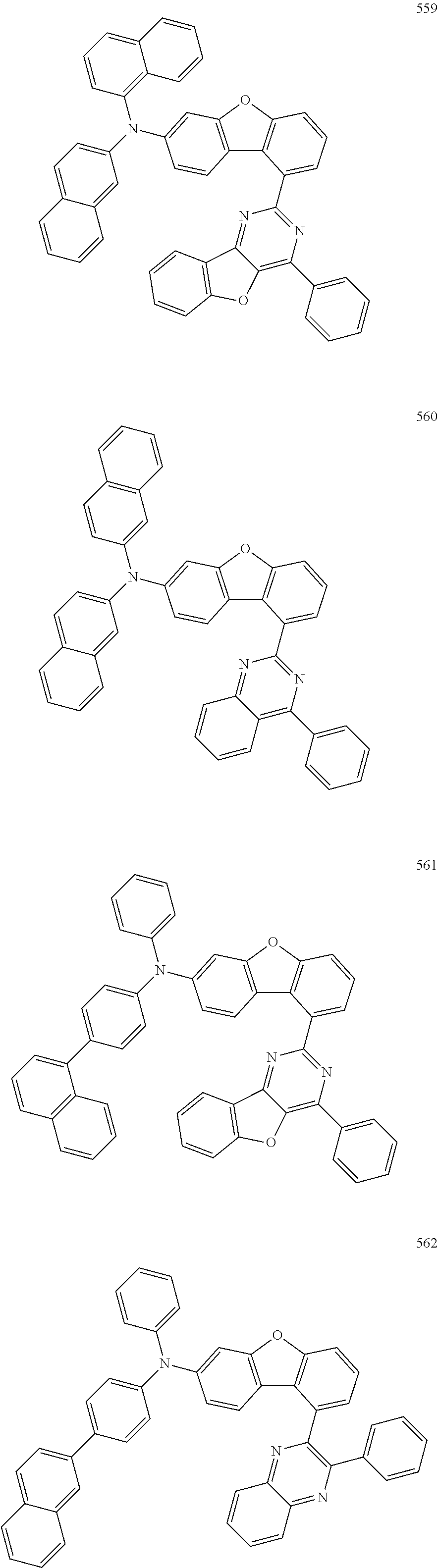 [SMILES: c1ccc(-c2nc(-c3cccc4oc5cc(N(c6ccc7ccccc7c6)c6cccc7ccccc67)ccc5c34)nc3c2oc2ccccc23)cc1 ; c1ccc(-c2nc(-c3cccc4oc5cc(N(c6ccc7ccccc7c6)c6ccc7ccccc7c6)ccc5c34)nc3ccccc23)cc1 ; c1ccc(-c2nc(-c3cccc4oc5cc(N(c6ccccc6)c6ccc(-c7cccc8ccccc78)cc6)ccc5c34)nc3c2oc2ccccc23)cc1 ; c1ccc(-c2nc3ccccc3nc2-c2cccc3oc4cc(N(c5ccccc5)c5ccc(-c6ccc7ccccc7c6)cc5)ccc4c23)cc1]

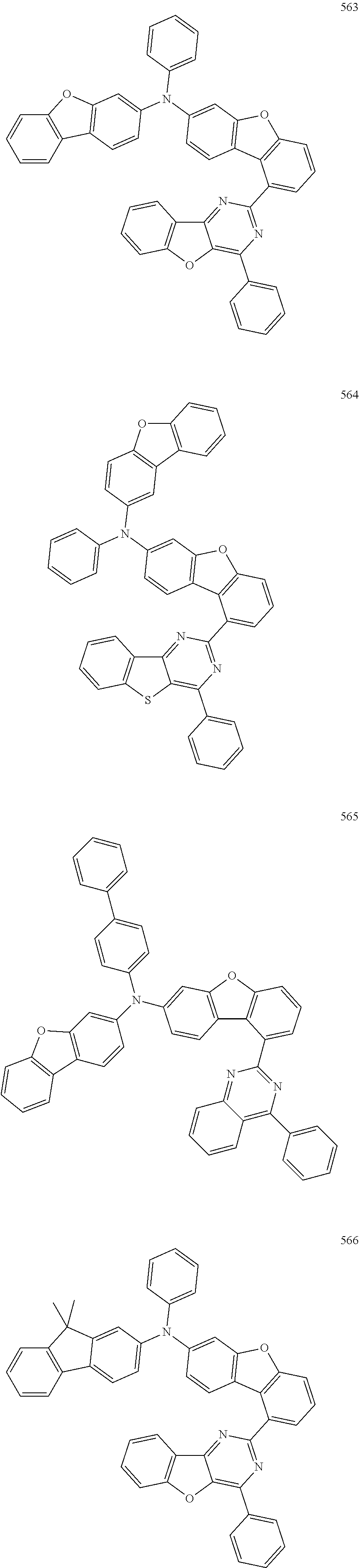 [SMILES: c1ccc(-c2nc(-c3cccc4oc5cc(N(c6ccccc6)c6ccc7c(c6)oc6ccccc67)ccc5c34)nc3c2oc2ccccc23)cc1 ; c1ccc(-c2nc(-c3cccc4oc5cc(N(c6ccccc6)c6ccc7oc8ccccc8c7c6)ccc5c34)nc3c2sc2ccccc23)cc1 ; c1ccc(-c2ccc(N(c3ccc4c(c3)oc3ccccc34)c3ccc4c(c3)oc3cccc(-c5nc(-c6ccccc6)c6ccccc6n5)c34)cc2)cc1 ; CC1(C)c2ccccc2-c2ccc(N(c3ccccc3)c3ccc4c(c3)oc3cccc(-c5nc(-c6ccccc6)c6oc7ccccc7c6n5)c34)cc21]

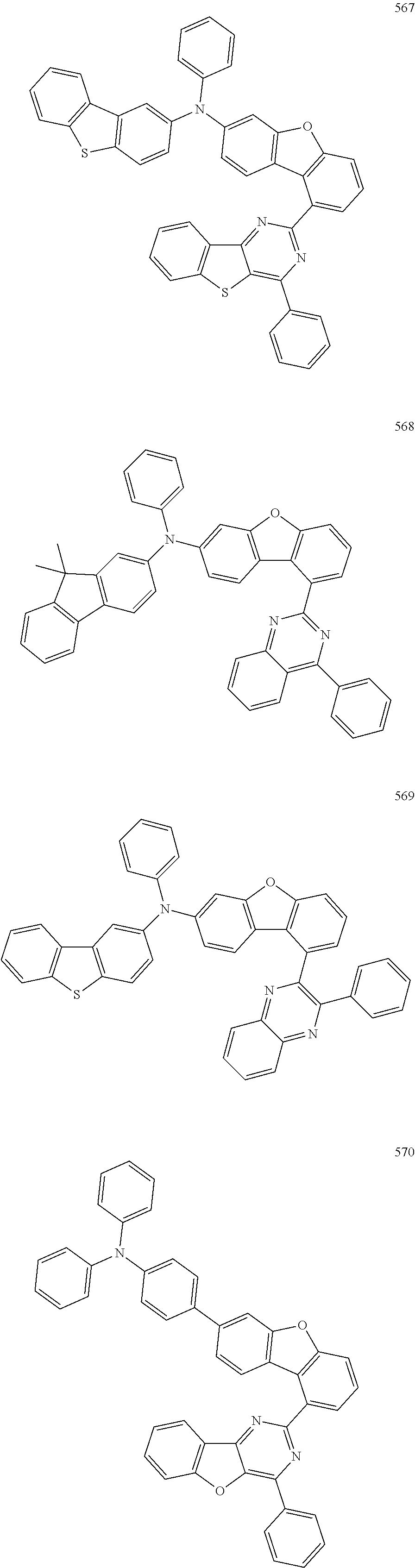 [SMILES: c1ccc(-c2nc(-c3cccc4oc5cc(N(c6ccccc6)c6ccc7sc8ccccc8c7c6)ccc5c34)nc3c2sc2ccccc23)cc1 ; CC1(C)c2ccccc2-c2ccc(N(c3ccccc3)c3ccc4c(c3)oc3cccc(-c5nc(-c6ccccc6)c6ccccc6n5)c34)cc21 ; c1ccc(-c2nc3ccccc3nc2-c2cccc3oc4cc(N(c5ccccc5)c5ccc6sc7ccccc7c6c5)ccc4c23)cc1 ; c1ccc(-c2nc(-c3cccc4oc5cc(-c6ccc(N(c7ccccc7)c7ccccc7)cc6)ccc5c34)nc3c2oc2ccccc23)cc1]

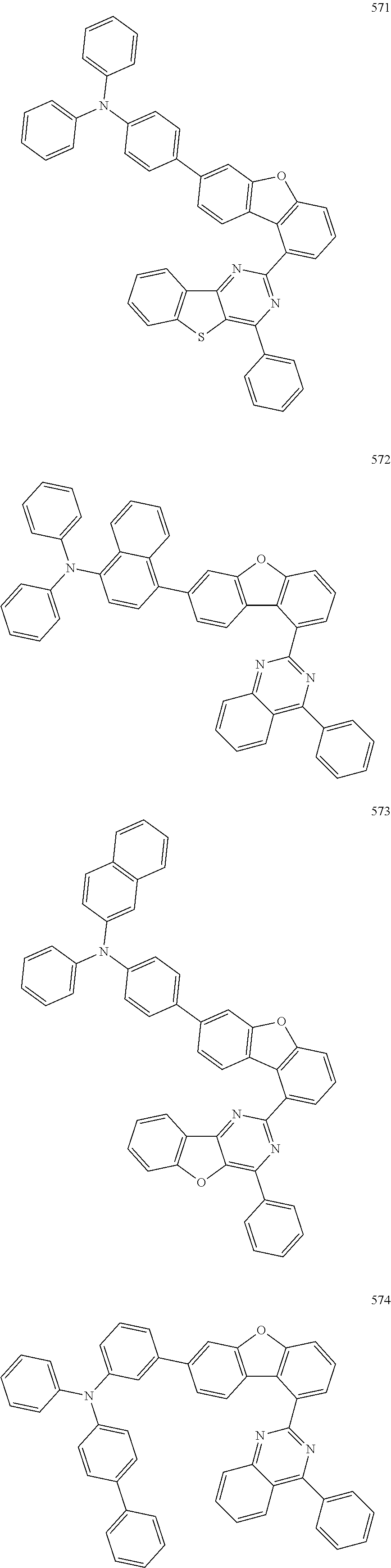 [SMILES: c1ccc(-c2nc(-c3cccc4oc5cc(-c6ccc(N(c7ccccc7)c7ccccc7)cc6)ccc5c34)nc3c2sc2ccccc23)cc1 ; c1ccc(-c2nc(-c3cccc4oc5cc(-c6ccc(N(c7ccccc7)c7ccccc7)c7ccccc67)ccc5c34)nc3ccccc23)cc1 ; c1ccc(-c2nc(-c3cccc4oc5cc(-c6ccc(N(c7ccccc7)c7ccc8ccccc8c7)cc6)ccc5c34)nc3c2oc2ccccc23)cc1 ; c1ccc(-c2ccc(N(c3ccccc3)c3cccc(-c4ccc5c(c4)oc4cccc(-c6nc(-c7ccccc7)c7ccccc7n6)c45)c3)cc2)cc1]

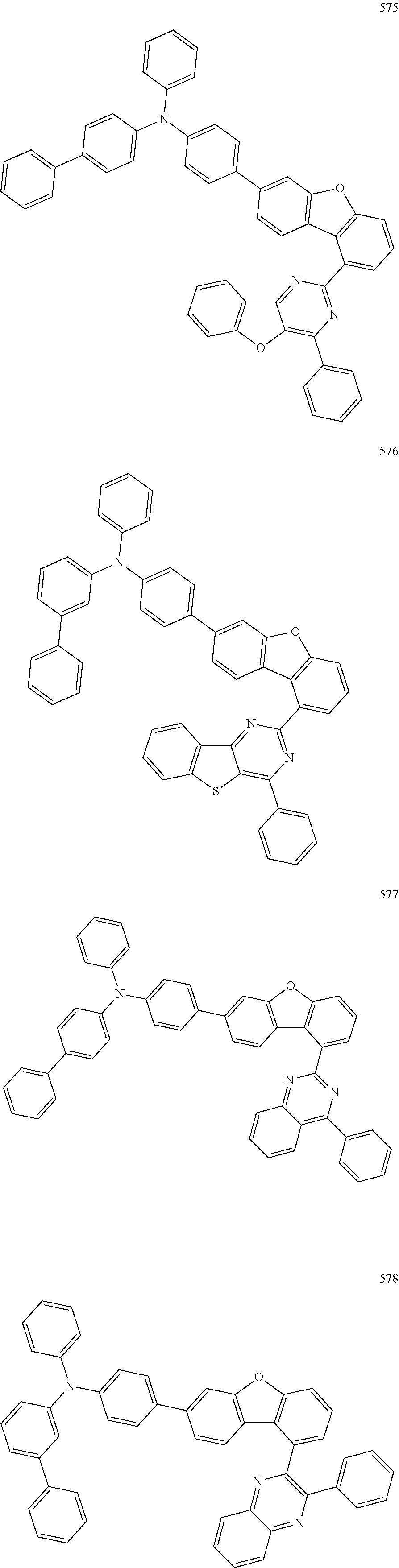 [SMILES: c1ccc(-c2ccc(N(c3ccccc3)c3ccc(-c4ccc5c(c4)oc4cccc(-c6nc(-c7ccccc7)c7oc8ccccc8c7n6)c45)cc3)cc2)cc1 ; c1ccc(-c2cccc(N(c3ccccc3)c3ccc(-c4ccc5c(c4)oc4cccc(-c6nc(-c7ccccc7)c7sc8ccccc8c7n6)c45)cc3)c2)cc1 ; c1ccc(-c2ccc(N(c3ccccc3)c3ccc(-c4ccc5c(c4)oc4cccc(-c6nc(-c7ccccc7)c7ccccc7n6)c45)cc3)cc2)cc1 ; c1ccc(-c2cccc(N(c3ccccc3)c3ccc(-c4ccc5c(c4)oc4cccc(-c6nc7ccccc7nc6-c6ccccc6)c45)cc3)c2)cc1]

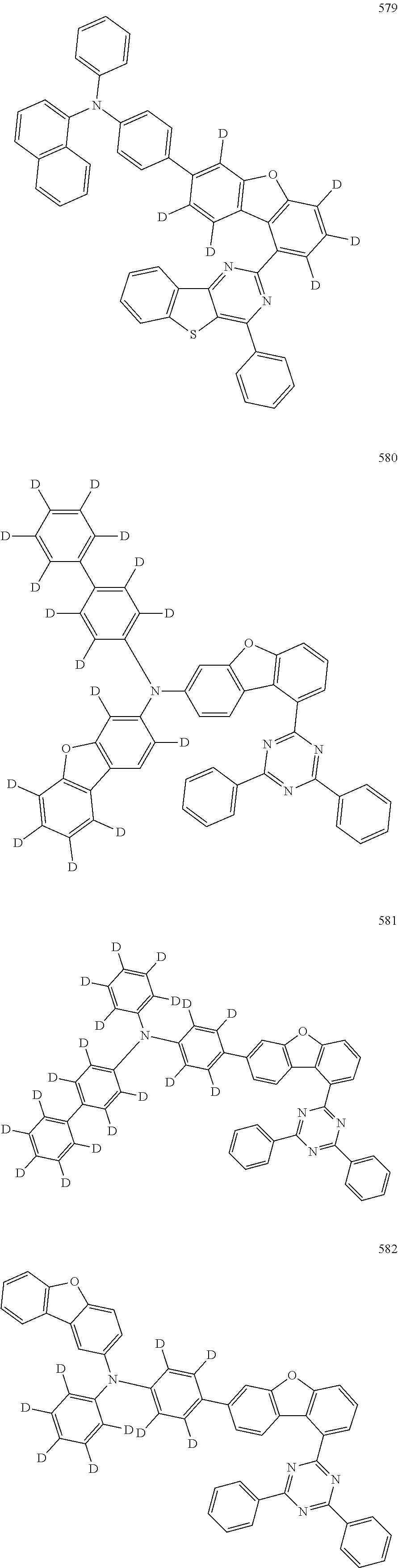

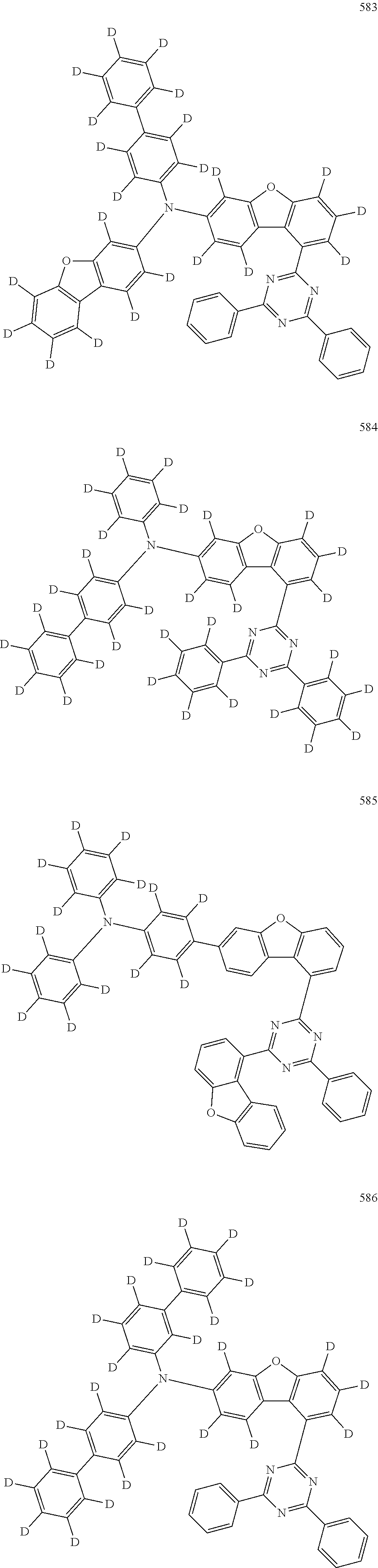

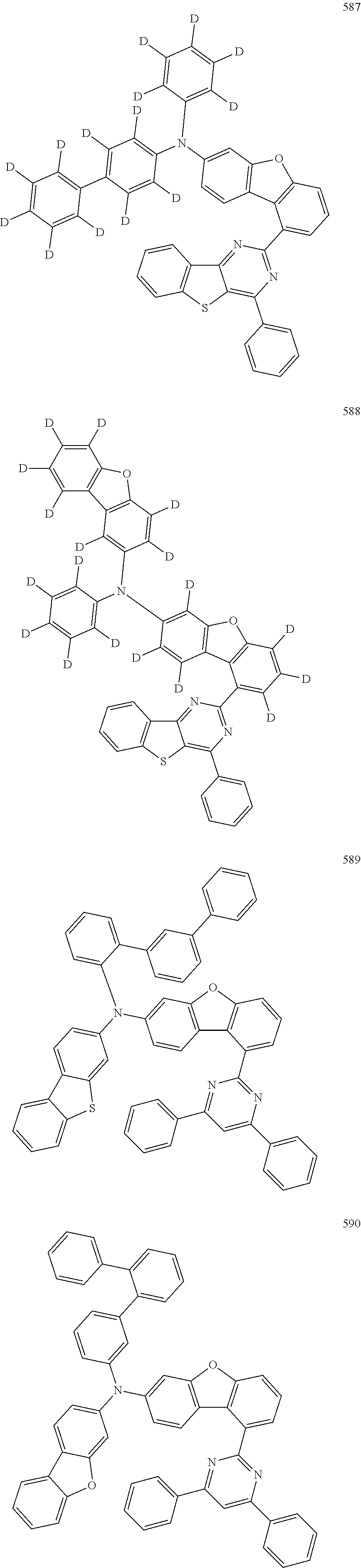

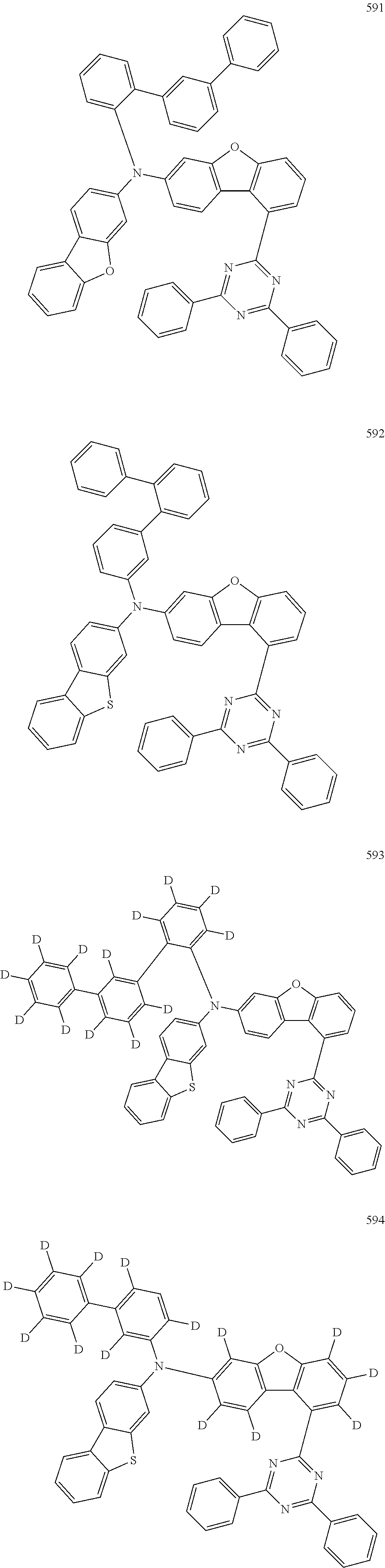

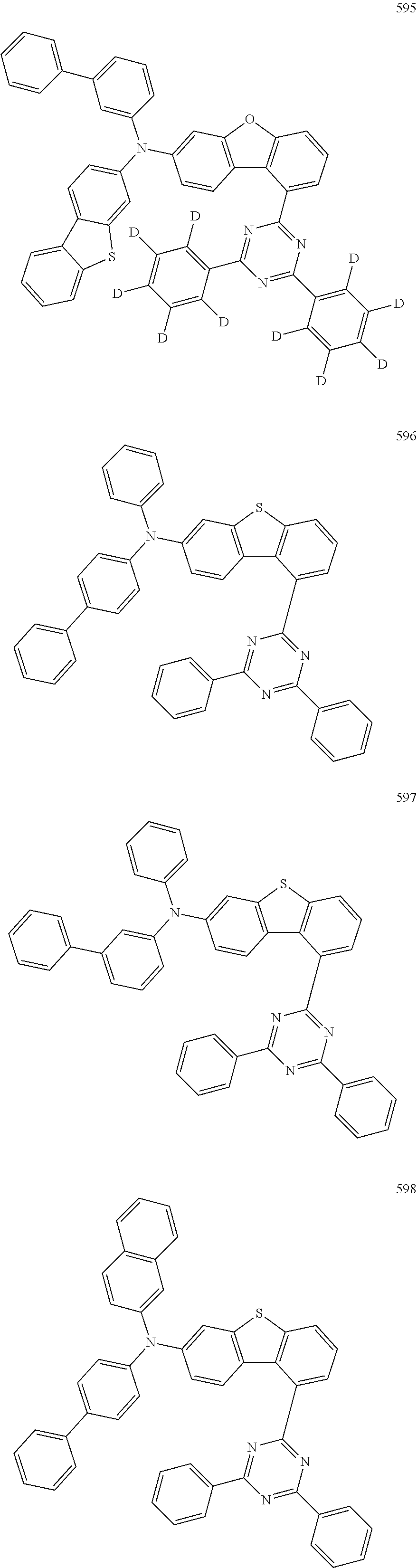 [SMILES: [2H]c1c([2H])c([2H])c(-c2nc(-c3c([2H])c([2H])c([2H])c([2H])c3[2H])nc(-c3cccc4oc5cc(N(c6cccc(-c7ccccc7)c6)c6ccc7c(c6)sc6ccccc67)ccc5c34)n2)c([2H])c1[2H] ; c1ccc(-c2ccc(N(c3ccccc3)c3ccc4c(c3)sc3cccc(-c5nc(-c6ccccc6)nc(-c6ccccc6)n5)c34)cc2)cc1 ; c1ccc(-c2cccc(N(c3ccccc3)c3ccc4c(c3)sc3cccc(-c5nc(-c6ccccc6)nc(-c6ccccc6)n5)c34)c2)cc1 ; c1ccc(-c2ccc(N(c3ccc4ccccc4c3)c3ccc4c(c3)sc3cccc(-c5nc(-c6ccccc6)nc(-c6ccccc6)n5)c34)cc2)cc1]

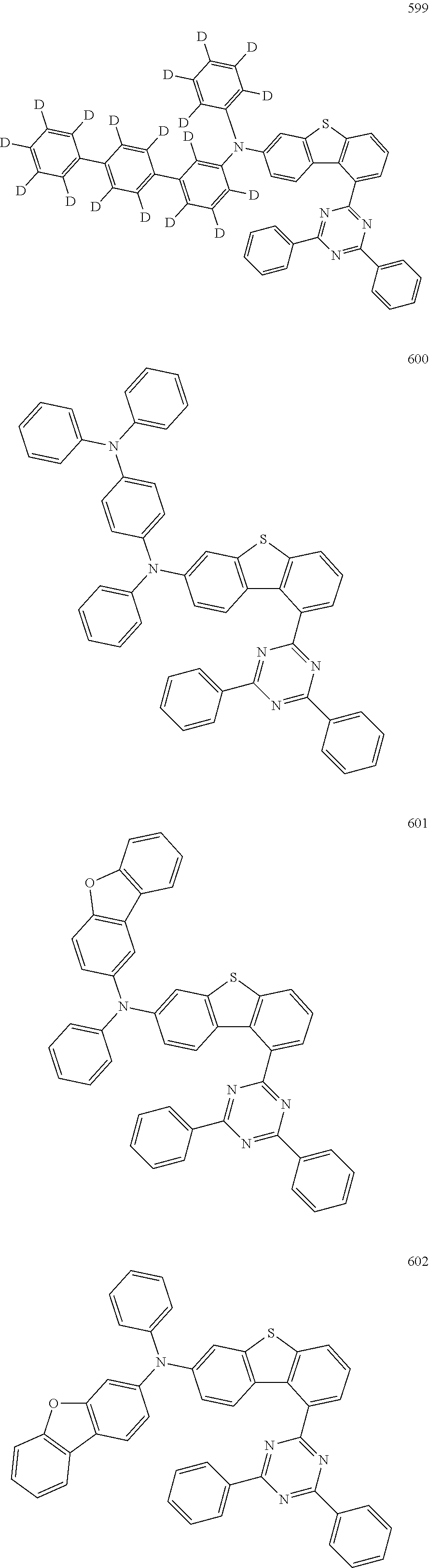 [SMILES: [2H]c1c([2H])c([2H])c(-c2c([2H])c([2H])c(-c3c([2H])c([2H])c([2H])c(N(c4ccc5c(c4)sc4cccc(-c6nc(-c7ccccc7)nc(-c7ccccc7)n6)c45)c4c([2H])c([2H])c([2H])c([2H])c4[2H])c3[2H])c([2H])c2[2H])c([2H])c1[2H] ; c1ccc(-c2nc(-c3ccccc3)nc(-c3cccc4sc5cc(N(c6ccccc6)c6ccc(N(c7ccccc7)c7ccccc7)cc6)ccc5c34)n2)cc1 ; c1ccc(-c2nc(-c3ccccc3)nc(-c3cccc4sc5cc(N(c6ccccc6)c6ccc7oc8ccccc8c7c6)ccc5c34)n2)cc1 ; c1ccc(-c2nc(-c3ccccc3)nc(-c3cccc4sc5cc(N(c6ccccc6)c6ccc7c(c6)oc6ccccc67)ccc5c34)n2)cc1]

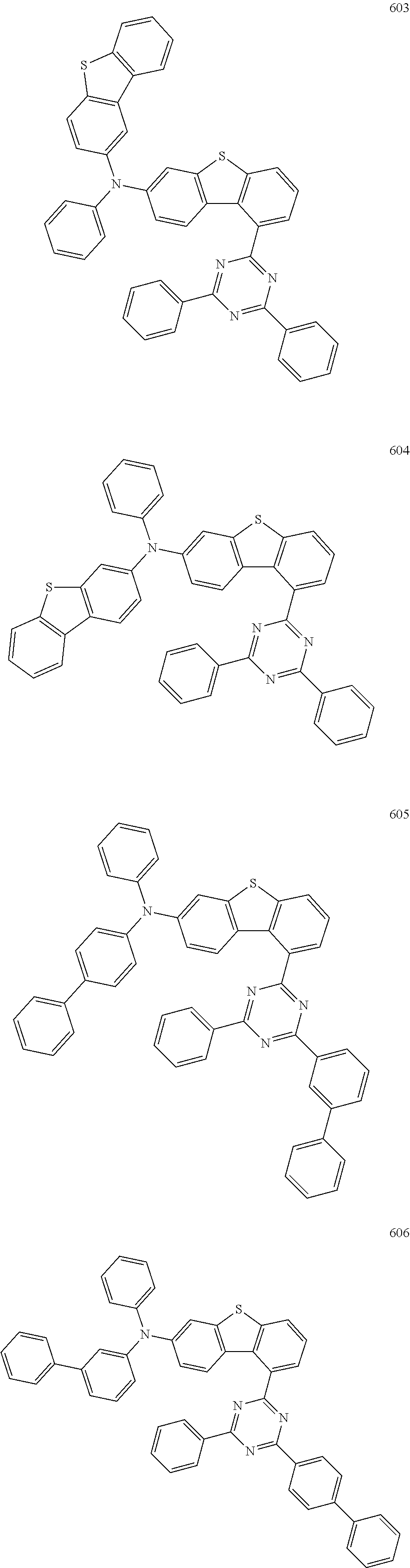 [SMILES: c1ccc(-c2nc(-c3ccccc3)nc(-c3cccc4sc5cc(N(c6ccccc6)c6ccc7sc8ccccc8c7c6)ccc5c34)n2)cc1 ; c1ccc(-c2nc(-c3ccccc3)nc(-c3cccc4sc5cc(N(c6ccccc6)c6ccc7c(c6)sc6ccccc67)ccc5c34)n2)cc1 ; c1ccc(-c2ccc(N(c3ccccc3)c3ccc4c(c3)sc3cccc(-c5nc(-c6ccccc6)nc(-c6cccc(-c7ccccc7)c6)n5)c34)cc2)cc1 ; c1ccc(-c2ccc(-c3nc(-c4ccccc4)nc(-c4cccc5sc6cc(N(c7ccccc7)c7cccc(-c8ccccc8)c7)ccc6c45)n3)cc2)cc1]

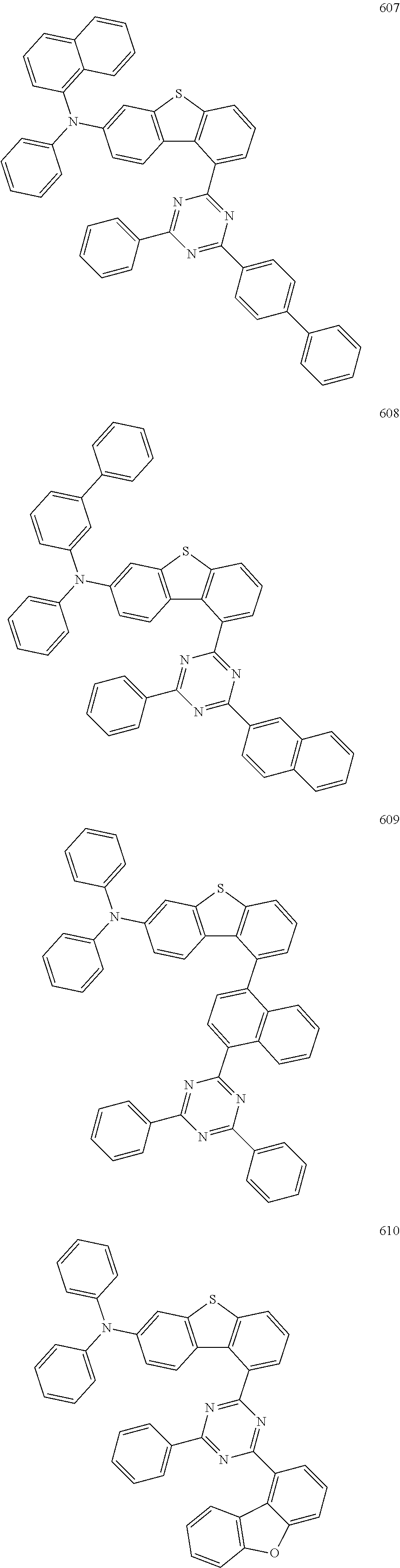 [SMILES: c1ccc(-c2ccc(-c3nc(-c4ccccc4)nc(-c4cccc5sc6cc(N(c7ccccc7)c7cccc8ccccc78)ccc6c45)n3)cc2)cc1 ; c1ccc(-c2cccc(N(c3ccccc3)c3ccc4c(c3)sc3cccc(-c5nc(-c6ccccc6)nc(-c6ccc7ccccc7c6)n5)c34)c2)cc1 ; c1ccc(-c2nc(-c3ccccc3)nc(-c3ccc(-c4cccc5sc6cc(N(c7ccccc7)c7ccccc7)ccc6c45)c4ccccc34)n2)cc1 ; c1ccc(-c2nc(-c3cccc4oc5ccccc5c34)nc(-c3cccc4sc5cc(N(c6ccccc6)c6ccccc6)ccc5c34)n2)cc1]

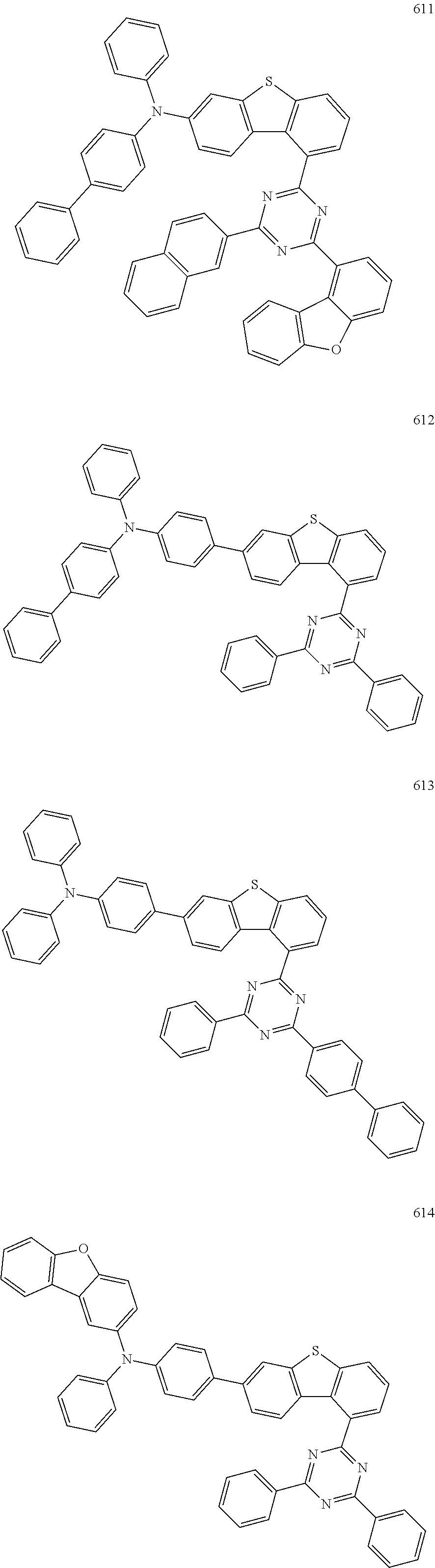 [SMILES: c1ccc(-c2ccc(N(c3ccccc3)c3ccc4c(c3)sc3cccc(-c5nc(-c6ccc7ccccc7c6)nc(-c6cccc7oc8ccccc8c67)n5)c34)cc2)cc1 ; c1ccc(-c2ccc(N(c3ccccc3)c3ccc(-c4ccc5c(c4)sc4cccc(-c6nc(-c7ccccc7)nc(-c7ccccc7)n6)c45)cc3)cc2)cc1 ; c1ccc(-c2ccc(-c3nc(-c4ccccc4)nc(-c4cccc5sc6cc(-c7ccc(N(c8ccccc8)c8ccccc8)cc7)ccc6c45)n3)cc2)cc1 ; c1ccc(-c2nc(-c3ccccc3)nc(-c3cccc4sc5cc(-c6ccc(N(c7ccccc7)c7ccc8oc9ccccc9c8c7)cc6)ccc5c34)n2)cc1]

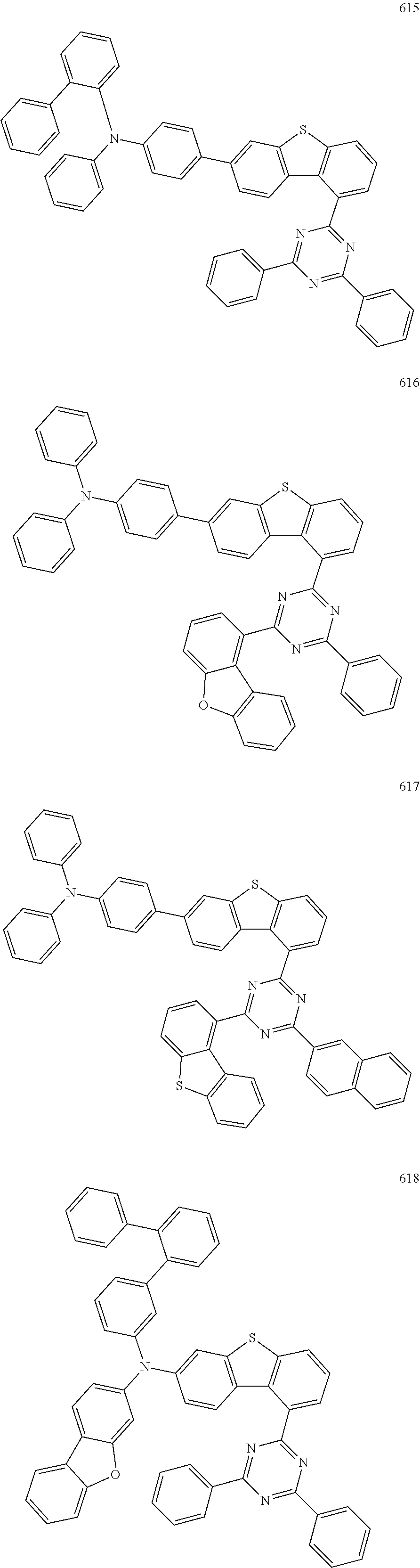 [SMILES: c1ccc(-c2nc(-c3ccccc3)nc(-c3cccc4sc5cc(-c6ccc(N(c7ccccc7)c7ccccc7-c7ccccc7)cc6)ccc5c34)n2)cc1 ; c1ccc(-c2nc(-c3cccc4oc5ccccc5c34)nc(-c3cccc4sc5cc(-c6ccc(N(c7ccccc7)c7ccccc7)cc6)ccc5c34)n2)cc1 ; c1ccc(N(c2ccccc2)c2ccc(-c3ccc4c(c3)sc3cccc(-c5nc(-c6ccc7ccccc7c6)nc(-c6cccc7sc8ccccc8c67)n5)c34)cc2)cc1 ; c1ccc(-c2nc(-c3ccccc3)nc(-c3cccc4sc5cc(N(c6cccc(-c7ccccc7-c7ccccc7)c6)c6ccc7c(c6)oc6ccccc67)ccc5c34)n2)cc1]

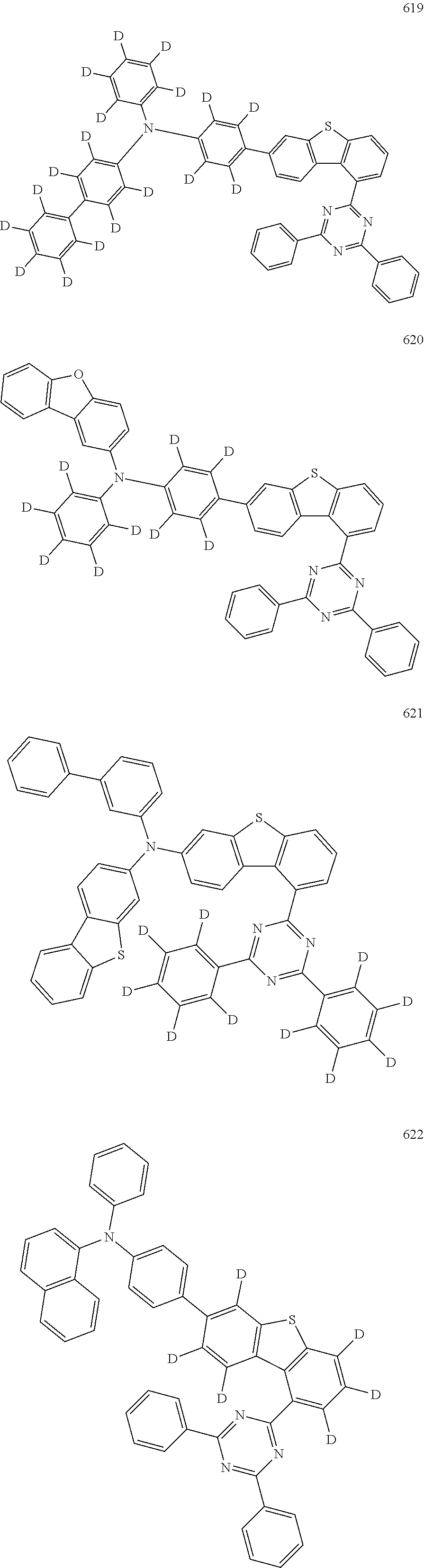

In an exemplary embodiment of the present application, the compound is just one example and is not limited thereto, and may include other compounds included in Chemical Formulae 1 and 2 which include an additional substituent. In addition, the substitution position of deuterium of the compound may be present while a specific position is excluded and hydrogen and deuterium are mixed during the process of deuterium substitution and synthesis.

Further, it is possible to synthesize a compound having inherent characteristics of a substituent introduced by introducing various substituents into the structures of Chemical Formulae 1 and 2. For example, a substituent usually used for a hole injection material, a hole transport material, a light emitting material, an electron transport material and an electron injection material, which are used when manufacturing an organic light emitting device, may be introduced into the core structure to synthesize a material which satisfies conditions required for each organic material layer.

In addition, by introducing various substituents into the structures of Chemical Formulae 1 and 2 or changing the binding position, the bandgap may be finely adjusted, and meanwhile, the characteristics at the interface between the organic material layers may be improved.

Furthermore, the compounds of Chemical Formulae 1 and 2 have excellent thermal stability, and such thermal stability provides driving stability to the organic light emitting device and improves service life characteristics.

In an exemplary embodiment of the present application, the organic material layer includes a light emitting layer, and the light emitting layer may include the compound of Chemical Formula 1 and the compound of Chemical Formula 2. That is, the compounds of Chemical Formulae 1 and 2 may be used as a light emitting material for the light emitting layer of the organic light emitting device.

In another exemplary embodiment, the compounds of Chemical Formulae 1 and 2 may be used as host materials for the light emitting layer of the organic light emitting device.

In an exemplary embodiment of the present application, the first electrode may be a positive electrode, and the second electrode may be a negative electrode.

In another exemplary embodiment, the first electrode may be a negative electrode, and the second electrode may be a positive electrode.

In an exemplary embodiment of the present application, the organic light emitting device may be a blue organic light emitting device, and the compounds according to Chemical Formulae 1 and 2 may be used as materials for the blue organic light emitting device.

In an exemplary embodiment of the present application, the organic light emitting device may be a green organic light emitting device, and the compounds according to Chemical Formulae 1 and 2 may be used as materials for the green organic light emitting device.

In an exemplary embodiment of the present application, the organic light emitting device may be a red organic light emitting device, and the compounds according to Chemical Formulae 1 and 2 may be used as materials for the red organic light emitting device.

In an exemplary embodiment of the present application, the organic light emitting device may be a blue organic light emitting device, and the compounds according to Chemical Formulae 1 and 2 may be used as materials for the light emitting layer of the blue organic light emitting device.

In an exemplary embodiment of the present application, the organic light emitting device may be a green organic light emitting device, and the compounds according to Chemical Formulae 1 and 2 may be used as materials for the light emitting layer of the green organic light emitting device.

In an exemplary embodiment of the present application, the organic light emitting device may be a red organic light emitting device, and the compounds according to Chemical Formulae 1 and 2 may be used as materials for the light emitting layer of the red organic light emitting device.

The organic light emitting device of the present invention may be manufactured using typical manufacturing methods and materials of an organic light emitting device, except that the above-described heterocyclic compound is used to form an organic material layer having one or more layers.

The heterocyclic compound may be formed as an organic material layer by not only a vacuum deposition method, but also a solution application method when an organic light emitting device is manufactured. Here, the solution application method means spin coating, dip coating, inkjet printing, screen printing, a spray method, roll coating, and the like, but is not limited thereto.

The organic material layer of the organic light emitting device of the present invention may be composed of a single-layered structure, but may be composed of a multi-layered structure in which two or more organic material layers are stacked. For example, the organic light emitting device of the present invention may have a structure including a hole injection layer, a hole transport layer, a light emitting layer, an electron transport layer, an electron injection layer, and the like as organic material layers. However, the structure of the organic light emitting device is not limited thereto, and may include a fewer number of organic material layers.

In an exemplary embodiment of the present application, as the iridium-based dopant, Ir(ppy)₃, which is a green phosphorescent dopant, may be used.

In an exemplary embodiment of the present application, as the iridium-based dopant, (piq)₂(Ir) (acac), which is a red phosphorescent dopant, may be used.

In the organic light emitting device of the present application, the organic material layer includes an electron injection layer or an electron transport layer, and the electron injection layer or the electron transport layer may include the compound of Chemical Formula 1, the compound of Chemical Formula 2, or a combination thereof.

In another organic light emitting device, the organic material layer includes a hole blocking layer, and the hole blocking layer may include the compound of Chemical Formula 1, the compound of Chemical Formula 2, or a combination thereof.

In still another organic light emitting device, the organic material layer includes an electron blocking layer, and the electron blocking layer may include the compound of Chemical Formula 1, the compound of Chemical Formula 2, or a combination thereof.

In yet another organic light emitting device, the organic material layer includes a hole transport layer, a light emitting layer, or an electron blocking layer, and the hole transport layer, the light emitting layer, or the electron blocking layer may include the compound of Chemical Formula 1, the compound of Chemical Formula 2, or a combination thereof.

In still yet another organic light emitting device, the organic material layer includes a hole transport layer or a hole transport auxiliary layer, and the hole transport layer or the hole transport auxiliary layer may include the compound of Chemical Formula 1, the compound of Chemical Formula 2, or a combination thereof.

In the organic light emitting device of the present application, as a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly[3,4-(ethylene-1,2-dioxy)thiophene](PEDOT), polypyrrole, and polyaniline; and the like, but are not limited thereto.

As a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrene-sulfonate), and the like.

As a hole transport material, a pyrazoline derivative, an arylamine-based derivative, a stilbene derivative, a triphenyldiamine derivative, and the like may be used, and a low-molecular weight or polymer material may also be used.

As an electron transport material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

As an electron injection material, for example, LiF is representatively used in the art, but the present application is not limited thereto.

As a light emitting material, a red, green, or blue light emitting material may be used, and if necessary, two or more light emitting materials may be mixed and used. In this case, two or more light emitting materials are deposited or used as an individual supply source, or pre-mixed to be deposited and used as one supply source.

Further, a fluorescent material may also be used as the light emitting material, but may also be used as a phosphorescent material. As the light emitting material, it is also possible to use alone a material which emits light by combining holes and electrons each injected from a positive electrode and a negative electrode, but materials in which a host material and a dopant material are involved in light emission together may also be used.

When hosts of the light emitting material are mixed and used, the same series of hosts may also be mixed and used, and different series of hosts may also be mixed and used. For example, two or more types of materials selected from n-type host materials or p-type host materials may be used as a host material for a light emitting layer.

The organic light emitting device according to an exemplary embodiment of the present application may be a top emission type, a bottom emission type, or a dual emission type according to the material to be used.

The compound of Chemical Formula 1 and the compound of Chemical Formula 2 according to an exemplary embodiment of the present application may act even in organic electronic devices including organic solar cells, organic photoconductors, organic transistors, and the like, based on the principle similar to those applied to organic light emitting devices.

The organic light emitting device of the present invention may further include one or two or more layers selected from the group consisting of a light emitting layer, a hole injection layer, a hole transport layer, a hole transport auxiliary layer, an electron blocking layer, a hole blocking layer, an electron transport layer and an electron injection layer.

In another exemplary embodiment, the compound represented by Chemical Formula 1 may be used as a light emitting material for a light emitting layer of an organic light emitting device, and may be used as an n-type host material.

In still another exemplary embodiment, the heterocyclic compound represented by Chemical Formula 2 may be used as a light emitting material for a light emitting layer of an organic light emitting device, and may be used as a p-type host material.

In the organic light emitting device of the present application, the organic material layer may include the compound represented by Chemical Formula 1 and the compound represented by Chemical Formula 2. The organic material layer may be formed by pre-mixing the compound represented by Chemical Formula 1 and the compound represented by Chemical Formula 2 and using a thermal vacuum deposition method.

In an exemplary embodiment of the present application, provided is a method for manufacturing an organic light emitting device, the method including: preparing a substrate; forming a first electrode on the substrate; forming an organic material layer having one or more layers on the first electrode; and forming a second electrode on the organic material layer, in which the forming of the organic material layer includes forming the organic material layer having one or more layers by using the composition for an organic material layer according to an exemplary embodiment of the present application.

In an exemplary embodiment of the present application, provided is a method for manufacturing an organic light emitting device, in which the forming of the organic material layer forms the organic material layer by supplying the compound represented by Chemical Formula 1 and the compound represented by Chemical Formula 2 as each individual supply source, and then using a thermal vacuum deposition method.

In an exemplary embodiment of the present application, provided is a method for manufacturing an organic light emitting device, in which the forming of the organic material layer forms the organic material layer by pre-mixing the compound represented by Chemical Formula 1 and the compound represented by Chemical Formula 2, and using a thermal vacuum deposition method.

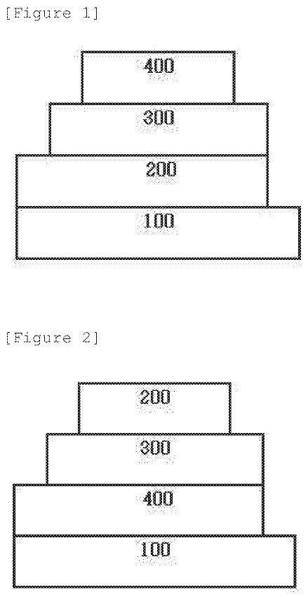

FIGS. 1 to 4 exemplify the stacking sequence of the electrodes and the organic material layer of the organic light emitting device according to an exemplary embodiment of the present application. However, the scope of the present application is not intended to be limited by these drawings, and the structure of the organic light emitting device known in the art may also be applied to the present application.

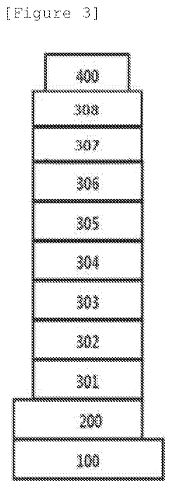

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as in FIG. 2, an organic light emitting device in which a negative electrode, an organic material layer, and a positive electrode are sequentially stacked on a substrate may also be implemented.

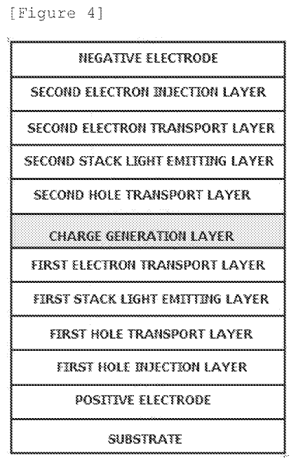

FIGS. 3 and 4 exemplify a case where an organic material layer is a multilayer.

An organic light emitting device according to FIG. 3 includes a hole injection layer 301, a hole transport layer 302, a hole transport auxiliary layer 303, an electron blocking layer 304, a light emitting layer 305, a hole blocking layer 306, an electron transport layer 307, and an electron injection layer 308.

Further, the organic light emitting device according to an exemplary embodiment of the present application includes a first electrode, a second electrode, and two or more stacks provided between the first electrode and the second electrode, the two or more stacks each independently include a light emitting layer, a charge generation layer is included between the two or more stacks, and the light emitting layer includes the compounds represented by Chemical Formulae 1 and 2.

The charge generation layer may include materials known in the art, for example, Bphen, MoO₃, and the like may be used, and the charge generation layer may be formed as a multi-layered structure.

In addition, the organic light emitting device according to an exemplary embodiment of the present application includes a first electrode, a first stack provided on the first electrode and including a first light emitting layer, a charge generation layer provided on the first stack, a second stack provided on the charge generation layer and including a second light emitting layer, and a second electrode provided on the second stack. In this case, the first light emitting layer and the second light emitting layer may include the compounds represented by Chemical Formulae 1 and 2. Furthermore, the first stack and the second stack may each independently further include one or more of the above-described hole injection layer, hole transport layer, hole blocking layer, electron transport layer, electron injection layer, and the like.

The charge generation layer may be an N-type charge generation layer, and the charge generation layer may additionally include a dopant known in the art.

As the organic light emitting device according to an exemplary embodiment of the present application, an organic light emitting device having a 2-stack tandem structure is schematically illustrated in FIG. 4.

In an exemplary embodiment of the present application, provided is a composition for forming an organic material layer including the compound of Chemical Formula 1 and the compound of Chemical Formula 2.

The compound of Chemical Formula 1 and the compound of Chemical Formula 2 of the composition for forming an organic material layer are the same as the definitions in Compounds 1 and 2 described above.

The composition for forming an organic material layer according to an exemplary embodiment of the present application may include the compound of Chemical Formula 1 and the compound of Chemical Formula 2 at a weight ratio of 1:10 to 10:1, specifically at a weight ratio of 1:5 to 5:1 and 1:3 to 3:1.

The composition for forming an organic material layer according to an exemplary embodiment of the present application may be used as a material for a light emitting layer of an organic light emitting device.

The composition may be used when an organic material layer of an organic light emitting device is formed, and particularly, may be more preferably used as a host material for the light emitting layer.

The composition is in a form in which two or more compounds are simply mixed, materials in a powder state may also be mixed before an organic material layer of an organic light emitting device is formed, and it is possible to mix compounds in a liquid state at a temperature which is equal to or more than a suitable temperature. The composition is in a solid state at a temperature which is equal to or less than the melting point of each material, and may be maintained as a liquid phase when the temperature is adjusted.

The composition may additionally include materials publicly known in the art such as solvents and additives.

Hereinafter, the present specification will be described in more detail through Examples, but these Examples are provided only for exemplifying the present application, and are not intended to limit the scope of the present application.

### [Preparation Example 1] Preparation of Target Compounds

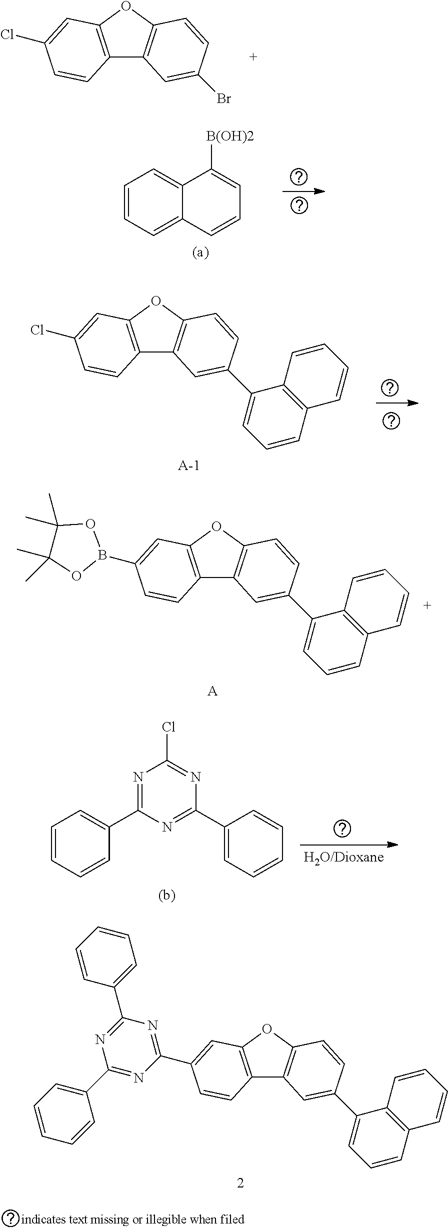

1) Preparation of Compound A-1

After 10.0 g (3.552 mmol) of (2-bromo-7-chlorodibenzo[b,d]furan, 3.12 g (4.262 mmol) of naphthalen-1-ylboronic acid, 0.21 g (0.177 mmol) of Pd(pph₃)₄, and 0.98 g (7.104 mmol) of K₂CO₃ were dissolved in 100 ml of 1,4-dioxane and 20 ml of H₂O, the resulting solution was refluxed at 120° C. for 1 hour and 30 minutes. After the reaction was completed, distilled water and DCM were added thereto at room temperature, extraction was performed, the organic layer was dried over MgSO₄, and then the solvent was removed using a rotary evaporator. The reaction product was purified by column chromatography (DCM:Hex=1:5) to obtain 10.5 g (89.9%) of Target Compound A-1.

2) Preparation of Compound A

After 10.5 g (31.93 mmol) of Compound A-1, 10.54 g (41.51 mmol) of 4,4,4′,4′,5,5,5′,5′-octamethyl-2,2′-bi(1,3,2-dioxaborolane), 1.46 g (1.596 mmol) of Pd₂(dba)₃, 1.31 g (3.193 mmol) of Sphos, and 8.83 g (63.86 mmol) of potassium acetate were dissolved in 105 mL of 1,4-dioxane, the resulting solution was refluxed at 120° C. for 5 hours. After the reaction was completed, distilled water and DCM were added thereto at room temperature, extraction was performed, the organic layer was dried over MgSO₄, and then the solvent was removed using a rotary evaporator. The reaction product was purified by column chromatography (DCM:Hex=1:6) to obtain 11 g (81.97%) of Target Compound A.

3) Preparation of Compound 2

After 11 g (26.17 mmol) of Compound A, 7.0 g (26.17 mmol) of 2-chloro-4,6-diphenyl-1,3,5-triazine, 1.51 g (1.308 mmol) of Pd(pph₃)₄, and 7.23 g (52.34 mmol) of potassium carbonate were dissolved in 110 ml of 1,4-dioxane and 22 ml of H₂O, the resulting solution was refluxed for 6 hours. After the reaction was completed, the resulting solid was filtered, washed with MeOH, and dried. The dried solid was dissolved in chloroform and purified by silica, and then the solvent was removed by a rotary evaporator. The residue was recrystallized with acetone to obtain 10 g (72.72%) of Target Compound 2.

The following target compounds were synthesized in the same manner as in Preparation Example 1, except that Intermediates (a) and (b) in the following Table 1 were used as intermediates in Preparation Example 1.

The following target compounds were synthesized in the same manner as in Preparation Example 1, except that 2-bromo-7-chlorodibenzo[b,d]thiophene was used instead of 2-bromo-7-chlorodibenzo[b,d]furan in Preparation Example 1.

TABLE 1

| Com- | | | | |
|---|---|---|---|---|
| pound | | | Target | |
| No. | Intermediate (a) | Intermediate (b) | compound | Yield |
| 2 | 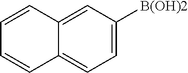 [SMILES: OB(O)c1ccc2ccccc2c1] | 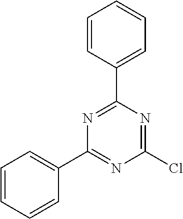 [SMILES: Clc1nc(-c2ccccc2)nc(-c2ccccc2)n1] | 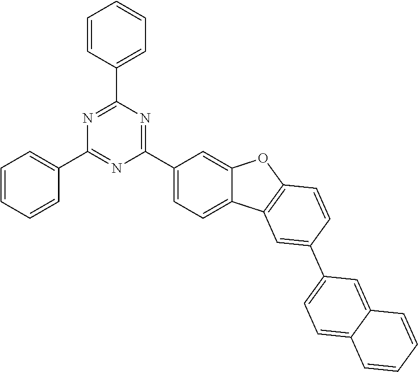 [SMILES: c1ccc(-c2nc(-c3ccccc3)nc(-c3ccc4c(c3)oc3ccc(-c5ccc6ccccc6c5)cc34)n2)cc1] | 50% |
| 19 | 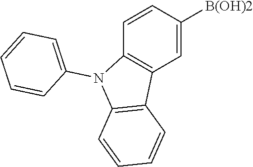 [SMILES: OB(O)c1ccc2c(c1)c1ccccc1n2-c1ccccc1] | 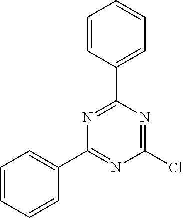 [SMILES: Clc1nc(-c2ccccc2)nc(-c2ccccc2)n1] | 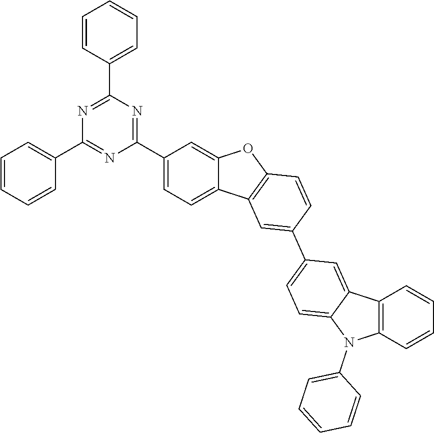 [SMILES: c1ccc(-c2nc(-c3ccccc3)nc(-c3ccc4c(c3)oc3ccc(-c5ccc6c(c5)c5ccccc5n6-c5ccccc5)cc34)n2)cc1] | 57% |
| 28 | 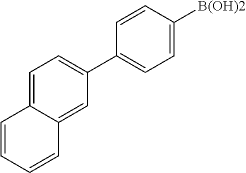 [SMILES: OB(O)c1ccc(-c2ccc3ccccc3c2)cc1] | 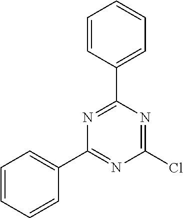 [SMILES: Clc1nc(-c2ccccc2)nc(-c2ccccc2)n1] | 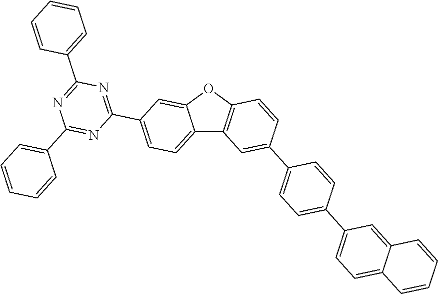 [SMILES: c1ccc(-c2nc(-c3ccccc3)nc(-c3ccc4c(c3)oc3ccc(-c5ccc(-c6ccc7ccccc7c6)cc5)cc34)n2)cc1] | 70% |
| 30 | 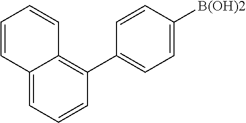 [SMILES: OB(O)c1ccc(-c2cccc3ccccc23)cc1] | 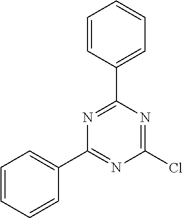 [SMILES: Clc1nc(-c2ccccc2)nc(-c2ccccc2)n1] | 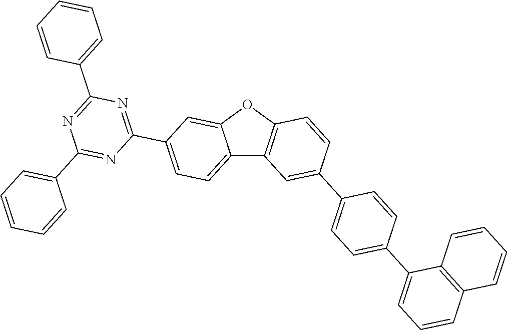 [SMILES: c1ccc(-c2nc(-c3ccccc3)nc(-c3ccc4c(c3)oc3ccc(-c5ccc(-c6cccc7ccccc67)cc5)cc34)n2)cc1] | 65% |
| 35 | 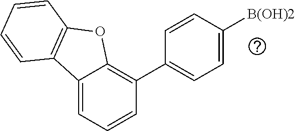 [SMILES: OB(O)c1ccc(-c2cccc3c2oc2ccccc23)cc1] | 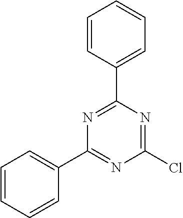 [SMILES: Clc1nc(-c2ccccc2)nc(-c2ccccc2)n1] | 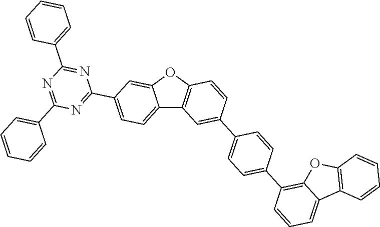 [SMILES: c1ccc(-c2nc(-c3ccccc3)nc(-c3ccc4c(c3)oc3ccc(-c5ccc(-c6cccc7c6oc6ccccc67)cc5)cc34)n2)cc1] | 59% |
| 51 | 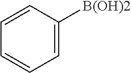 | 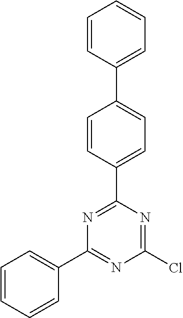 [SMILES: Clc1nc(-c2ccccc2)nc(-c2ccc(-c3ccccc3)cc2)n1] | 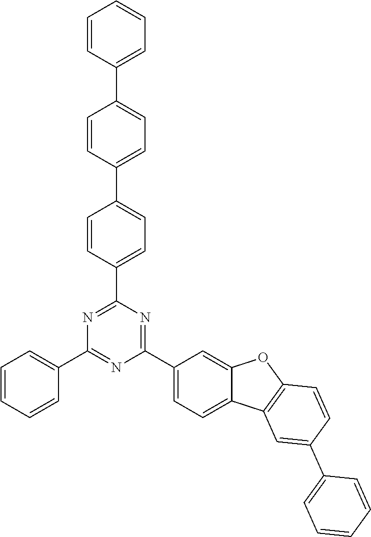 [SMILES: c1ccc(-c2ccc(-c3ccc(-c4nc(-c5ccccc5)nc(-c5ccc6c(c5)oc5ccc(-c7ccccc7)cc56)n4)cc3)cc2)cc1] | 78% |
| 55 | 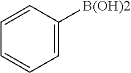 | 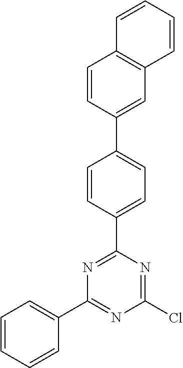 [SMILES: Clc1nc(-c2ccccc2)nc(-c2ccc(-c3ccc4ccccc4c3)cc2)n1] | 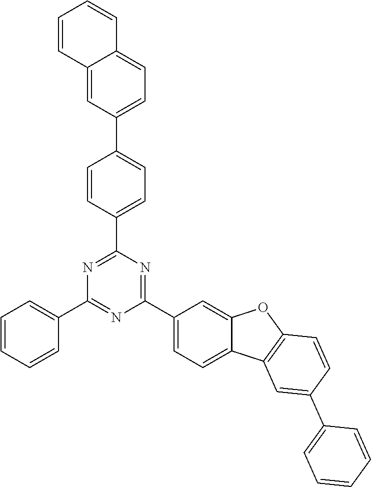 [SMILES: c1ccc(-c2ccc3oc4cc(-c5nc(-c6ccccc6)nc(-c6ccc(-c7ccc8ccccc8c7)cc6)n5)ccc4c3c2)cc1] | 66% |
| 69 | 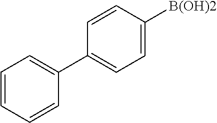 [SMILES: OB(O)c1ccc(-c2ccccc2)cc1] | 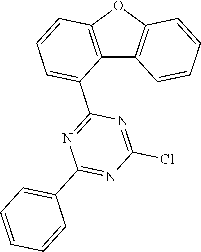 [SMILES: Clc1nc(-c2ccccc2)nc(-c2cccc3oc4ccccc4c23)n1] | 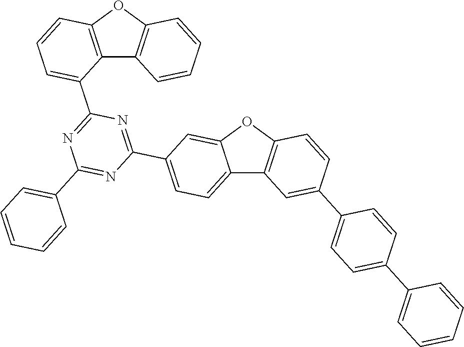 [SMILES: c1ccc(-c2ccc(-c3ccc4oc5cc(-c6nc(-c7ccccc7)nc(-c7cccc8oc9ccccc9c78)n6)ccc5c4c3)cc2)cc1] | 68% |
| 75 | 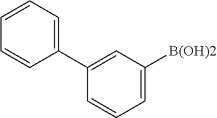 [SMILES: OB(O)c1cccc(-c2ccccc2)c1] | 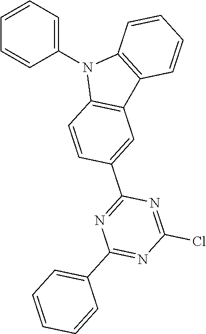 [SMILES: Clc1nc(-c2ccccc2)nc(-c2ccc3c(c2)c2ccccc2n3-c2ccccc2)n1] | 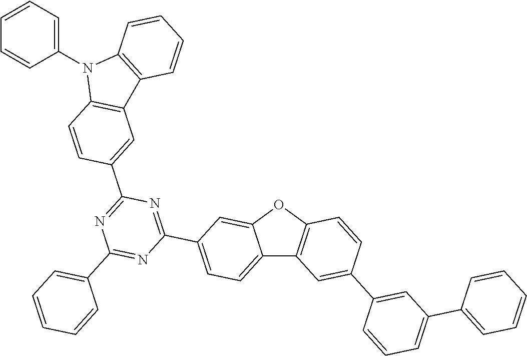 [SMILES: c1ccc(-c2cccc(-c3ccc4oc5cc(-c6nc(-c7ccccc7)nc(-c7ccc8c(c7)c7ccccc7n8-c7ccccc7)n6)ccc5c4c3)c2)cc1] | 45% |
| 83 | 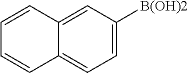 [SMILES: OB(O)c1ccc2ccccc2c1] | 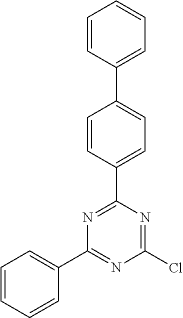 [SMILES: Clc1nc(-c2ccccc2)nc(-c2ccc(-c3ccccc3)cc2)n1] | 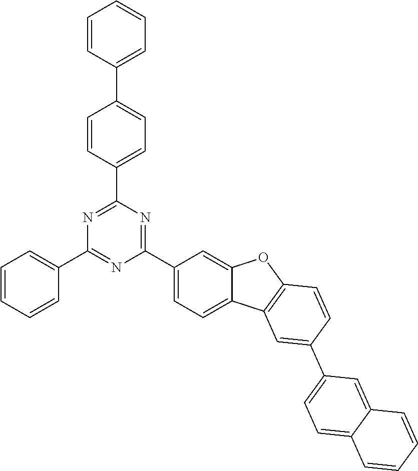 [SMILES: c1ccc(-c2ccc(-c3nc(-c4ccccc4)nc(-c4ccc5c(c4)oc4ccc(-c6ccc7ccccc7c6)cc45)n3)cc2)cc1] | 72% |
| 92 | 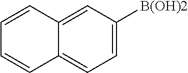 [SMILES: OB(O)c1ccc2ccccc2c1] | 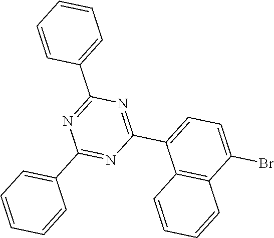 [SMILES: Brc1ccc(-c2nc(-c3ccccc3)nc(-c3ccccc3)n2)c2ccccc12] | 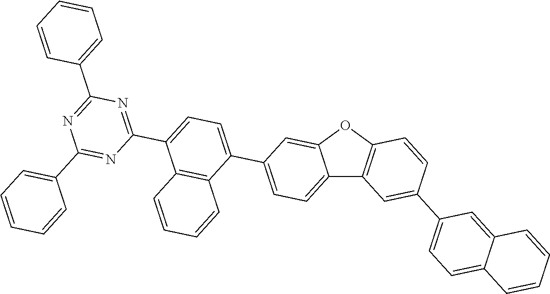 [SMILES: c1ccc(-c2nc(-c3ccccc3)nc(-c3ccc(-c4ccc5c(c4)oc4ccc(-c6ccc7ccccc7c6)cc45)c4ccccc34)n2)cc1] | 66% |
| 96 | 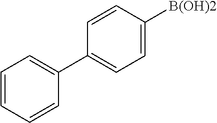 [SMILES: OB(O)c1ccc(-c2ccccc2)cc1] | 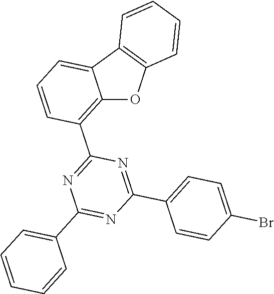 [SMILES: Brc1ccc(-c2nc(-c3ccccc3)nc(-c3cccc4c3oc3ccccc34)n2)cc1] | 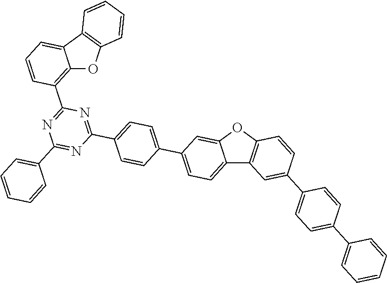 [SMILES: c1ccc(-c2ccc(-c3ccc4oc5cc(-c6ccc(-c7nc(-c8ccccc8)nc(-c8cccc9c8oc8ccccc89)n7)cc6)ccc5c4c3)cc2)cc1] | 73% |
| 126 | 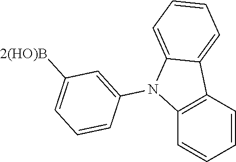 | 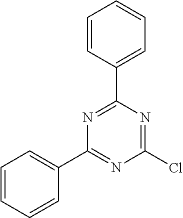 [SMILES: Clc1nc(-c2ccccc2)nc(-c2ccccc2)n1] | 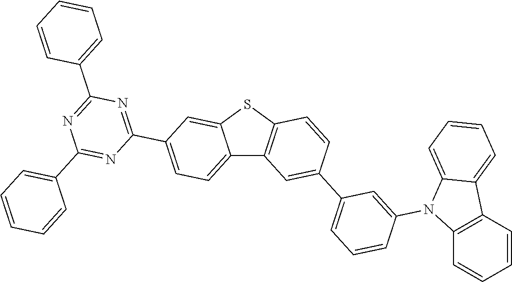 [SMILES: c1ccc(-c2nc(-c3ccccc3)nc(-c3ccc4c(c3)sc3ccc(-c5cccc(-n6c7ccccc7c7ccccc76)c5)cc34)n2)cc1] | 65% |
| 140 | 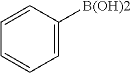 | 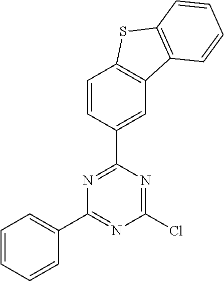 [SMILES: Clc1nc(-c2ccccc2)nc(-c2ccc3sc4ccccc4c3c2)n1] | 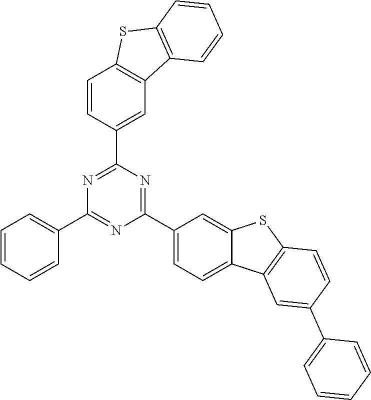 [SMILES: c1ccc(-c2ccc3sc4cc(-c5nc(-c6ccccc6)nc(-c6ccc7sc8ccccc8c7c6)n5)ccc4c3c2)cc1] | 62% |
| 154 | 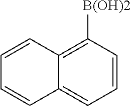 [SMILES: OB(O)c1cccc2ccccc12] | 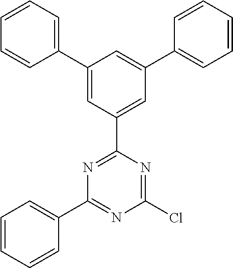 [SMILES: Clc1nc(-c2ccccc2)nc(-c2cc(-c3ccccc3)cc(-c3ccccc3)c2)n1] | 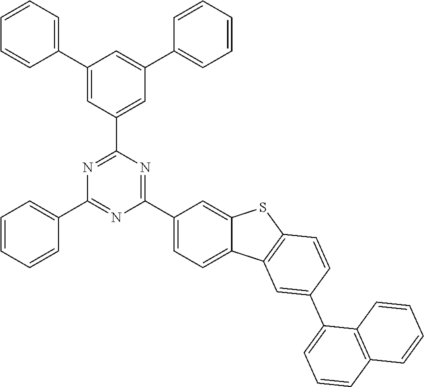 [SMILES: c1ccc(-c2cc(-c3ccccc3)cc(-c3nc(-c4ccccc4)nc(-c4ccc5c(c4)sc4ccc(-c6cccc7ccccc67)cc45)n3)c2)cc1] | 45% |
| 330 | 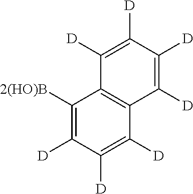 | 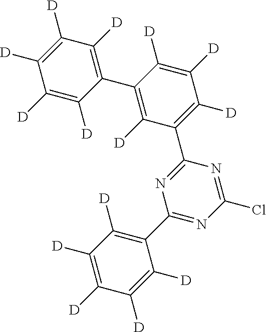 [SMILES: [2H]c1c([2H])c([2H])c(-c2nc(Cl)nc(-c3c([2H])c([2H])c([2H])c(-c4c([2H])c([2H])c([2H])c([2H])c4[2H])c3[2H])n2)c([2H])c1[2H]] | 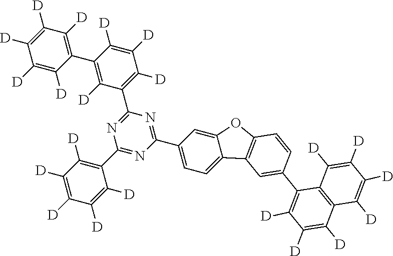 [SMILES: [2H]c1c([2H])c([2H])c(-c2nc(-c3ccc4c(c3)oc3ccc(-c5c([2H])c([2H])c([2H])c6c([2H])c([2H])c([2H])c([2H])c56)cc34)nc(-c3c([2H])c([2H])c([2H])c(-c4c([2H])c([2H])c([2H])c([2H])c4[2H])c3[2H])n2)c([2H])c1[2H]] | 66% |
| 4 | 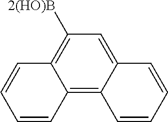 | 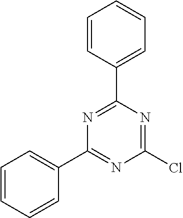 [SMILES: Clc1nc(-c2ccccc2)nc(-c2ccccc2)n1] | 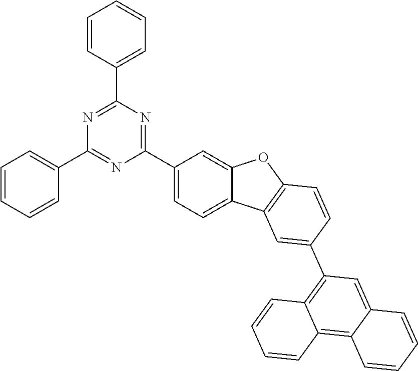 [SMILES: c1ccc(-c2nc(-c3ccccc3)nc(-c3ccc4c(c3)oc3ccc(-c5cc6ccccc6c6ccccc56)cc34)n2)cc1] | 72% |
| 12 | 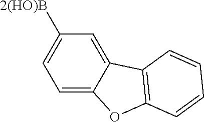 | 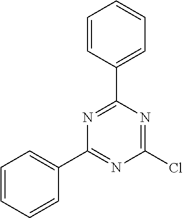 [SMILES: Clc1nc(-c2ccccc2)nc(-c2ccccc2)n1] | 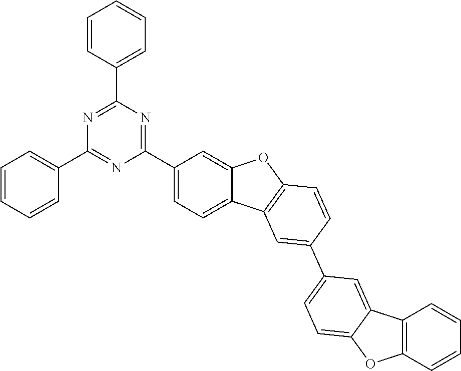 [SMILES: c1ccc(-c2nc(-c3ccccc3)nc(-c3ccc4c(c3)oc3ccc(-c5ccc6oc7ccccc7c6c5)cc34)n2)cc1] | 78% |
| 22 | 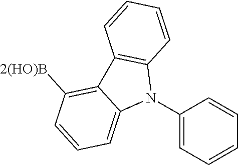 | 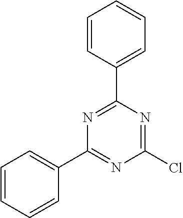 [SMILES: Clc1nc(-c2ccccc2)nc(-c2ccccc2)n1] | 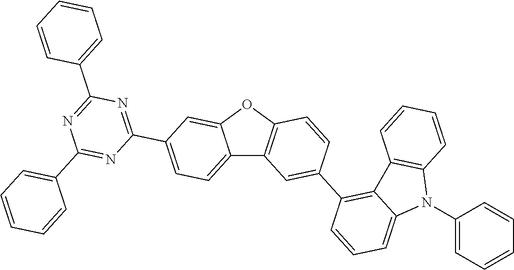 [SMILES: c1ccc(-c2nc(-c3ccccc3)nc(-c3ccc4c(c3)oc3ccc(-c5cccc6c5c5ccccc5n6-c5ccccc5)cc34)n2)cc1] | 67% |
| 651 | 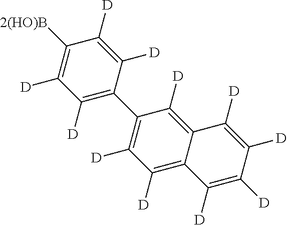 | 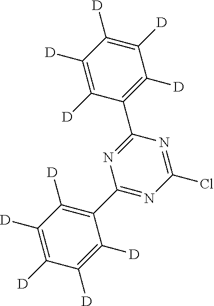 [SMILES: [2H]c1c([2H])c([2H])c(-c2nc(Cl)nc(-c3c([2H])c([2H])c([2H])c([2H])c3[2H])n2)c([2H])c1[2H]] | 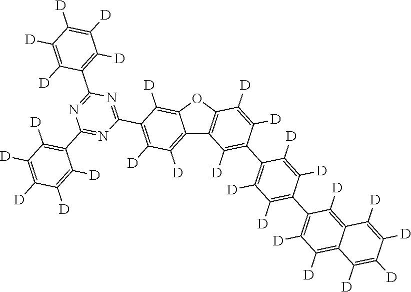 [SMILES: [2H]c1c([2H])c([2H])c(-c2nc(-c3c([2H])c([2H])c([2H])c([2H])c3[2H])nc(-c3c([2H])c([2H])c4c(oc5c([2H])c([2H])c(-c6c([2H])c([2H])c(-c7c([2H])c([2H])c8c([2H])c([2H])c([2H])c([2H])c8c7[2H])c([2H])c6[2H])c([2H])c54)c3[2H])n2)c([2H])c1[2H]] | 65% |
| 652 | 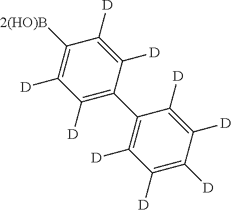 | 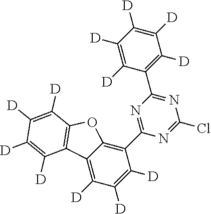 [SMILES: [2H]c1c([2H])c([2H])c(-c2nc(Cl)nc(-c3c([2H])c([2H])c([2H])c4c3oc3c([2H])c([2H])c([2H])c([2H])c34)n2)c([2H])c1[2H]] | 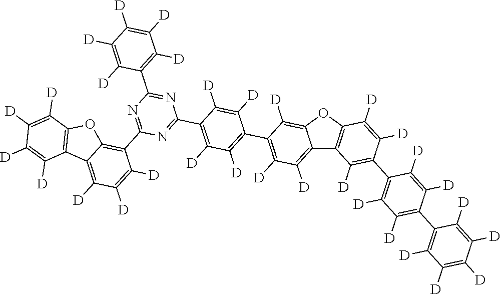 [SMILES: [2H]c1c([2H])c([2H])c(-c2nc(-c3c([2H])c([2H])c(-c4c([2H])c([2H])c5c(oc6c([2H])c([2H])c(-c7c([2H])c([2H])c(-c8c([2H])c([2H])c([2H])c([2H])c8[2H])c([2H])c7[2H])c([2H])c65)c4[2H])c([2H])c3[2H])nc(-c3c([2H])c([2H])c([2H])c4c3oc3c([2H])c([2H])c([2H])c([2H])c34)n2)c([2H])c1[2H]] | 61% |
| 44 | 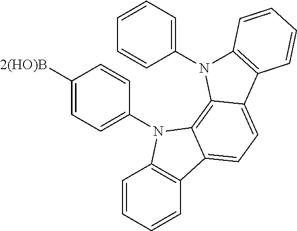 | 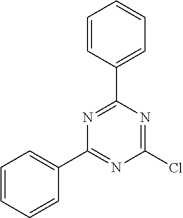 [SMILES: Clc1nc(-c2ccccc2)nc(-c2ccccc2)n1] | 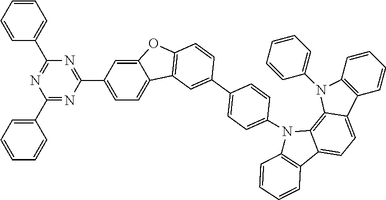 [SMILES: c1ccc(-c2nc(-c3ccccc3)nc(-c3ccc4c(c3)oc3ccc(-c5ccc(-n6c7ccccc7c7ccc8c9ccccc9n(-c9ccccc9)c8c76)cc5)cc34)n2)cc1] | 60% |
| 47 | 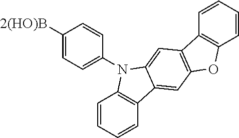 | 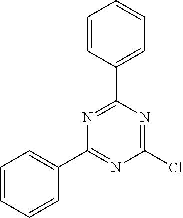 [SMILES: Clc1nc(-c2ccccc2)nc(-c2ccccc2)n1] | 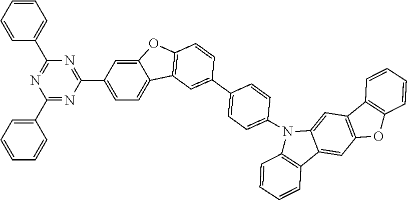 [SMILES: c1ccc(-c2nc(-c3ccccc3)nc(-c3ccc4c(c3)oc3ccc(-c5ccc(-n6c7ccccc7c7cc8oc9ccccc9c8cc76)cc5)cc34)n2)cc1] | 67% |

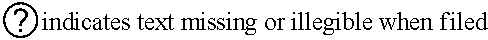

### [Preparation Example 2] Preparation of Target Compounds

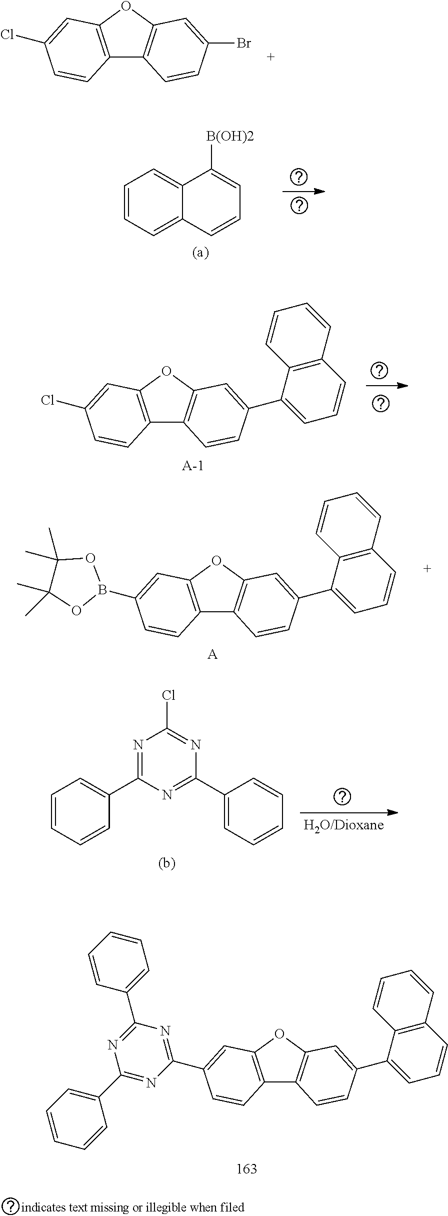

The following target compounds were synthesized in the same manner as in Preparation Example 1, except that 3-bromo-7-chlorodibenzo[b,d]furan and 3-bromo-7-chlorodibenzo[b,d]thiophene were used instead of 2-bromo-7-chlorodibenzo[b,d]furan in Preparation Example 1.

TABLE 2

| Com- | | | | |
|---|---|---|---|---|
| pound | | | | |
| No. | Intermediate (C) | Intermediate (d) | Target compound | Yield |
| 176 | 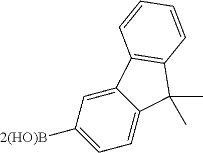 | 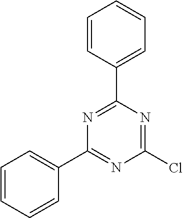 [SMILES: Clc1nc(-c2ccccc2)nc(-c2ccccc2)n1] | 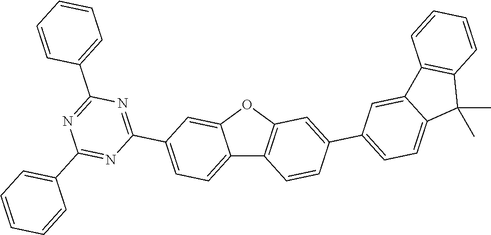 [SMILES: CC1(C)c2ccccc2-c2cc(-c3ccc4c(c3)oc3cc(-c5nc(-c6ccccc6)nc(-c6ccccc6)n5)ccc34)ccc21] | 50% |
| 206 | 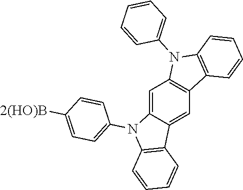 | 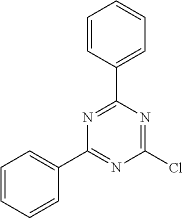 [SMILES: Clc1nc(-c2ccccc2)nc(-c2ccccc2)n1] | 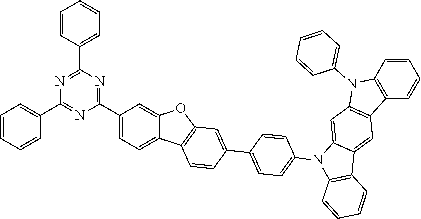 [SMILES: c1ccc(-c2nc(-c3ccccc3)nc(-c3ccc4c(c3)oc3cc(-c5ccc(-n6c7ccccc7c7cc8c9ccccc9n(-c9ccccc9)c8cc76)cc5)ccc34)n2)cc1] | 57% |
| 216 | 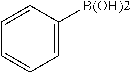 | 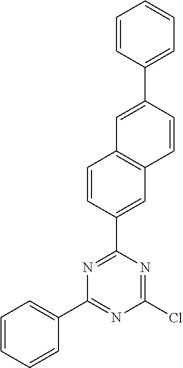 [SMILES: Clc1nc(-c2ccccc2)nc(-c2ccc3cc(-c4ccccc4)ccc3c2)n1] | 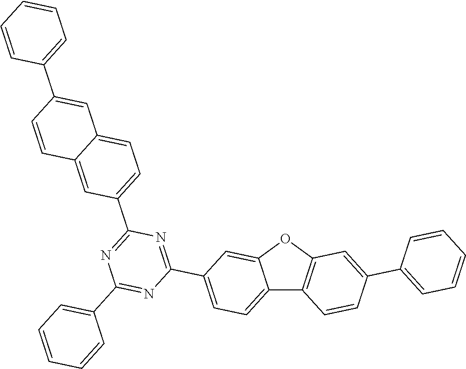 [SMILES: c1ccc(-c2ccc3cc(-c4nc(-c5ccccc5)nc(-c5ccc6c(c5)oc5cc(-c7ccccc7)ccc56)n4)ccc3c2)cc1] | 70% |
| 226 | 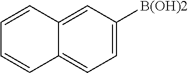 [SMILES: OB(O)c1ccc2ccccc2c1] | 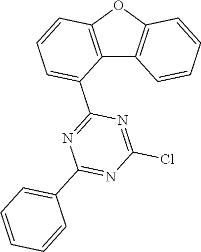 [SMILES: Clc1nc(-c2ccccc2)nc(-c2cccc3oc4ccccc4c23)n1] | 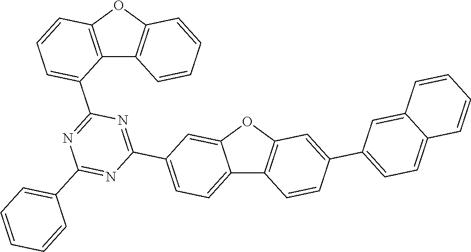 [SMILES: c1ccc(-c2nc(-c3ccc4c(c3)oc3cc(-c5ccc6ccccc6c5)ccc34)nc(-c3cccc4oc5ccccc5c34)n2)cc1] | 65% |
| 240 | 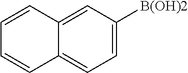 [SMILES: OB(O)c1ccc2ccccc2c1] | 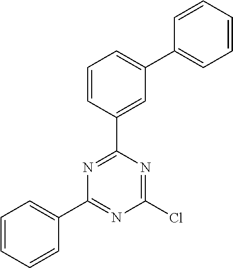 [SMILES: Clc1nc(-c2ccccc2)nc(-c2cccc(-c3ccccc3)c2)n1] | 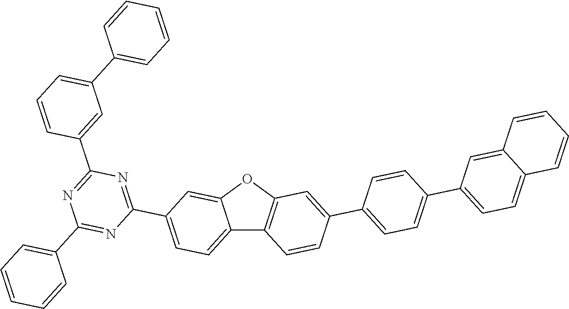 [SMILES: c1ccc(-c2cccc(-c3nc(-c4ccccc4)nc(-c4ccc5c(c4)oc4cc(-c6ccc(-c7ccc8ccccc8c7)cc6)ccc45)n3)c2)cc1] | 59% |
| 252 | 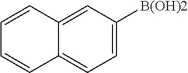 [SMILES: OB(O)c1ccc2ccccc2c1] | 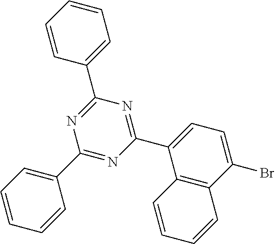 [SMILES: Brc1ccc(-c2nc(-c3ccccc3)nc(-c3ccccc3)n2)c2ccccc12] | 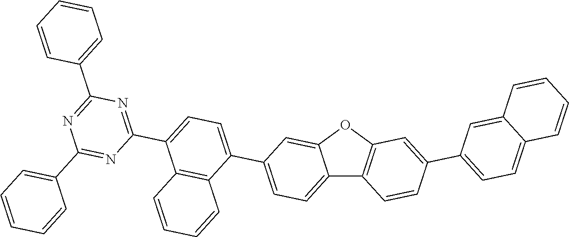 [SMILES: c1ccc(-c2nc(-c3ccccc3)nc(-c3ccc(-c4ccc5c(c4)oc4cc(-c6ccc7ccccc7c6)ccc45)c4ccccc34)n2)cc1] | 55% |
| 280 | 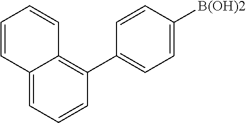 [SMILES: OB(O)c1ccc(-c2cccc3ccccc23)cc1] | 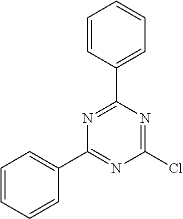 [SMILES: Clc1nc(-c2ccccc2)nc(-c2ccccc2)n1] | 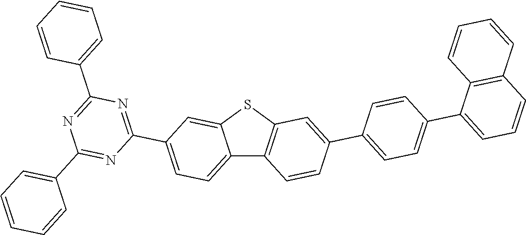 [SMILES: c1ccc(-c2nc(-c3ccccc3)nc(-c3ccc4c(c3)sc3cc(-c5ccc(-c6cccc7ccccc67)cc5)ccc34)n2)cc1] | 66% |
| 298 | 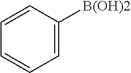 | 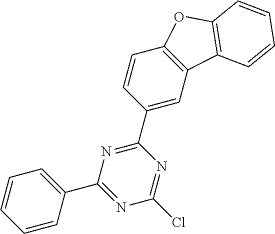 [SMILES: Clc1nc(-c2ccccc2)nc(-c2ccc3oc4ccccc4c3c2)n1] | 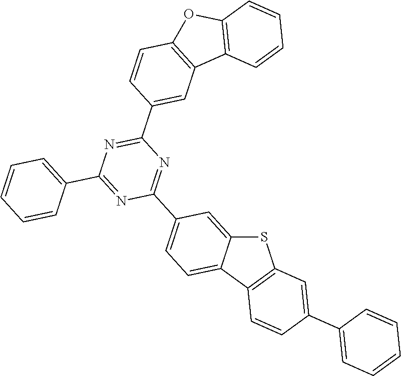 [SMILES: c1ccc(-c2ccc3c(c2)sc2cc(-c4nc(-c5ccccc5)nc(-c5ccc6oc7ccccc7c6c5)n4)ccc23)cc1] | 55% |
| 318 | 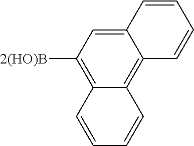 | 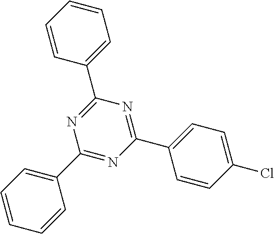 [SMILES: Clc1ccc(-c2nc(-c3ccccc3)nc(-c3ccccc3)n2)cc1] | 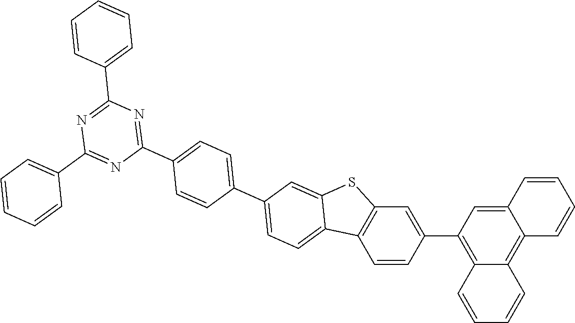 [SMILES: c1ccc(-c2nc(-c3ccccc3)nc(-c3ccc(-c4ccc5c(c4)sc4cc(-c6cc7ccccc7c7ccccc67)ccc45)cc3)n2)cc1] | 45% |
| 326 | 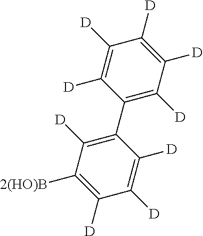 | 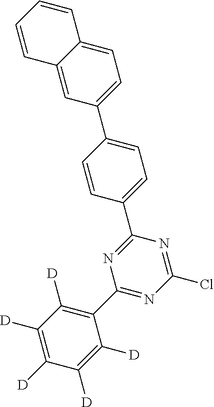 [SMILES: [2H]c1c([2H])c([2H])c(-c2nc(Cl)nc(-c3ccc(-c4ccc5ccccc5c4)cc3)n2)c([2H])c1[2H]] | 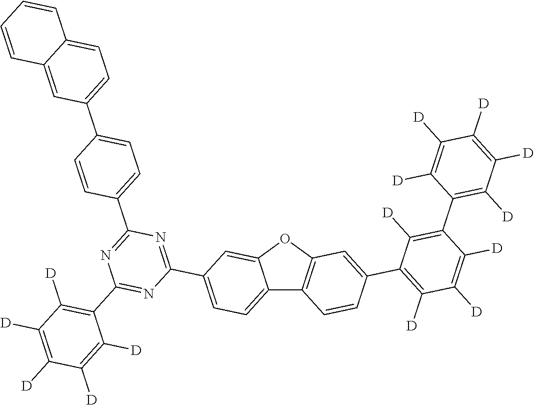 [SMILES: [2H]c1c([2H])c([2H])c(-c2nc(-c3ccc(-c4ccc5ccccc5c4)cc3)nc(-c3ccc4c(c3)oc3cc(-c5c([2H])c([2H])c([2H])c(-c6c([2H])c([2H])c([2H])c([2H])c6[2H])c5[2H])ccc34)n2)c([2H])c1[2H]] | 59% |
| 268 | 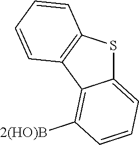 | 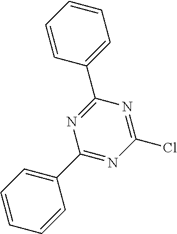 [SMILES: Clc1nc(-c2ccccc2)nc(-c2ccccc2)n1] | 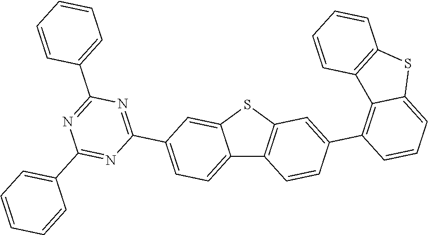 [SMILES: c1ccc(-c2nc(-c3ccccc3)nc(-c3ccc4c(c3)sc3cc(-c5cccc6sc7ccccc7c56)ccc34)n2)cc1] | 61% |

### [Preparation Example 3] Preparation of Target Compounds

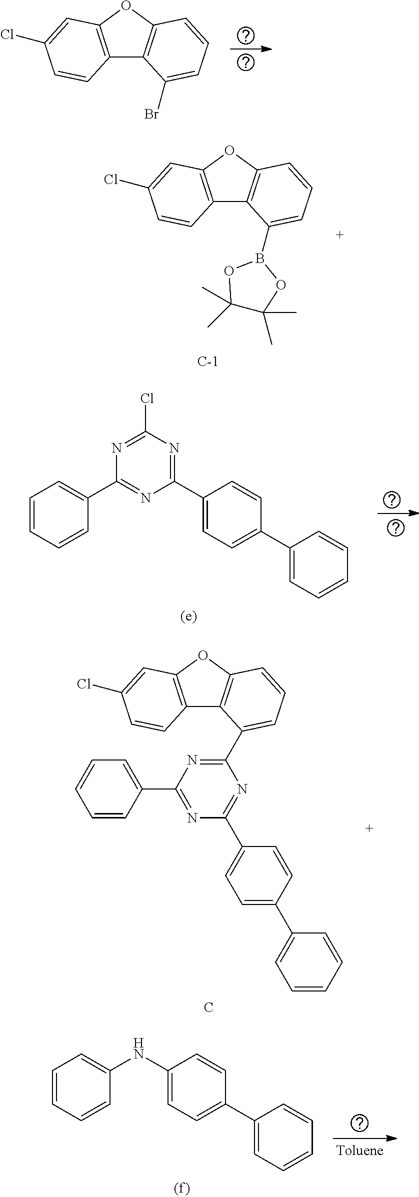

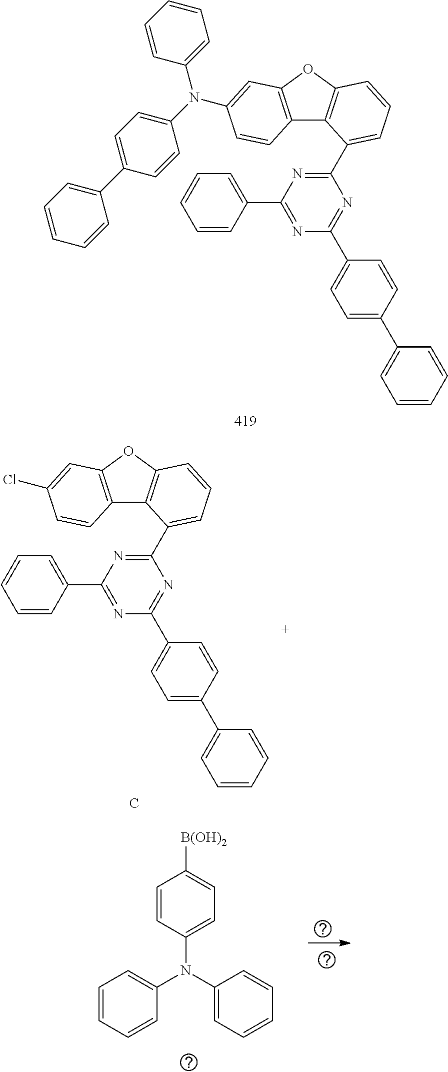

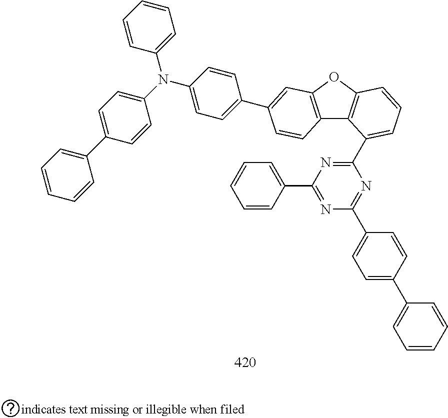 [SMILES: c1ccc(-c2ccc(-c3nc(-c4ccccc4)nc(-c4cccc5oc6cc(-c7ccc(N(c8ccccc8)c8ccc(-c9ccccc9)cc8)cc7)ccc6c45)n3)cc2)cc1 ; "420"]

1) Preparation of Compound C-1

After a compound 1-bromo-7-chlorodibenzo[b,d]furan (50.0 g, 178 mmol), B₂pin₂ (54.1 g, 213 mmol), Pd (dppf) Cl₂ (6.51 g, 8.90 mmol), and KOAc (34.9 g, 356 mmol) were dissolved in dioxane (500 mL), the resulting solution was refluxed for 4 hours. After the reaction was completed, distilled water and DCM were added thereto at room temperature, extraction was performed, the organic layer was dried over MgSO₄, and then the solvent was removed using a rotary evaporator. The reaction product was purified by column chromatography (DCM:Hex=1:3) to obtain Compound C-1 (44.0 g, 134 mmol, 75%) as a target compound.

2) Preparation of Compound C

After Compound C-1 (7.0 g, 21.3 mmol), a compound 2-([1,1′-biphenyl]-4-yl)-4-chloro-6-phenyl-1,3,5-triazine (7.18 g, 20.9 mmol), Pd(PPh₃)₄ (1.23 g, 1.07 mmol), and K₂CO₃ (5.89 g, 42.6 mmol) were dissolved in H₂O/1,4-dioxane (21 mL/70 mL), the resulting solution was refluxed for 2 hours. After the reaction was completed, the resulting solid was filtered, washed with MeOH, and dried. The dried solid was dissolved in chloroform and purified by silica, and then the solvent was removed by a rotary evaporator. The residue was recrystallized with acetone to obtain Compound C (10.4 g, 20.4 mmol, 96%) as a target compound.

3) Preparation of Compound 419

After Compound C (10.4 g, 20.4 mmol), N-phenyl-[1,1′-biphenyl]-4-amine (5.00 g, 20.4 mmol), Pd₂dba₃ (0.93 g, 1.02 mmol), Xphos (0.97 g, 2.04 mmol), and NaOtBu (3.92 g, 40.8 mmol) were dissolved in toluene (100 mL), the resulting solution was refluxed for 3 hours. After the reaction was completed, distilled water and DCM were added thereto at room temperature, extraction was performed, the organic layer was dried over MgSO₄, and then the solvent was removed by a rotary evaporator. The reaction product was purified by column chromatography (DCM:Hex=1:6) to obtain 11.6 g (76%) of Compound 419 as a target compound.

4) Preparation of Compound 498

After Compound C (7.0 g, 16.1 mmol), a compound 4-(diphenylamino)phenyl)boronic acid (6.50 g, 16.9 mmol), Pd₂dba₃ (0.74 g, 0.81 mmol), Xphos (0.77 g, 1.61 mmol), and NaOH (1.29 g, 32.2 mmol) were dissolved in H₂O/1,4-dioxane (21 mL/70 mL), the resulting solution was refluxed for 3 hours. After the reaction was completed, the resulting solid was filtered, washed with MeOH, and dried. The dried solid was dissolved in chloroform and purified by silica, and then the solvent was removed by a rotary evaporator. The residue was recrystallized with acetone to obtain Compound 498 (9.95 g, 13.8 mmol, 86%) as a target compound.

The following target compounds were synthesized in the same manner as in Preparation Example 3, except that Intermediates (e) and (f)/(f′) in the following Table 3 were used as intermediates in Preparation Example 3.

TABLE 3

| Com- | | | | |
|---|---|---|---|---|
| pound | | | | |
| No. | Intermediate (e) | Intermediate (f)/(f′) | Target compound | Yield |
| 332 | 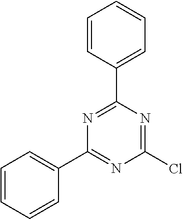 [SMILES: Clc1nc(-c2ccccc2)nc(-c2ccccc2)n1] | 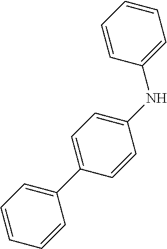 [SMILES: c1ccc(Nc2ccc(-c3ccccc3)cc2)cc1] | 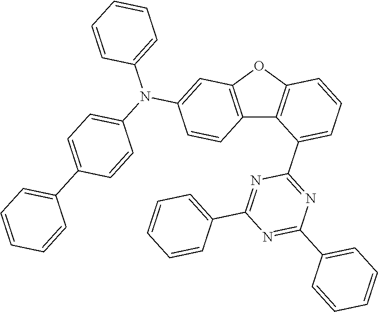 [SMILES: c1ccc(-c2ccc(N(c3ccccc3)c3ccc4c(c3)oc3cccc(-c5nc(-c6ccccc6)nc(-c6ccccc6)n5)c34)cc2)cc1] | 50% |
| 338 | 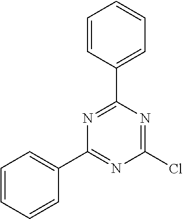 [SMILES: Clc1nc(-c2ccccc2)nc(-c2ccccc2)n1] | 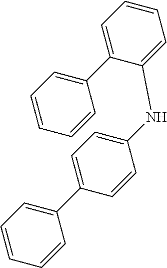 [SMILES: c1ccc(-c2ccc(Nc3ccccc3-c3ccccc3)cc2)cc1] | 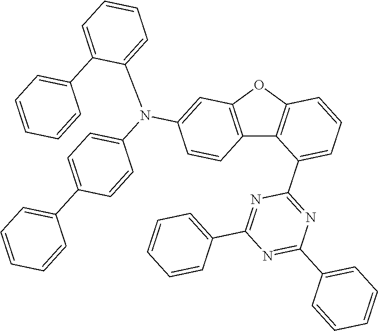 [SMILES: c1ccc(-c2ccc(N(c3ccc4c(c3)oc3cccc(-c5nc(-c6ccccc6)nc(-c6ccccc6)n5)c34)c3ccccc3-c3ccccc3)cc2)cc1] | 57% |
| 339 | 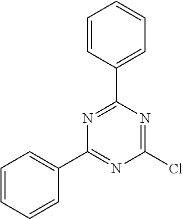 [SMILES: Clc1nc(-c2ccccc2)nc(-c2ccccc2)n1] | 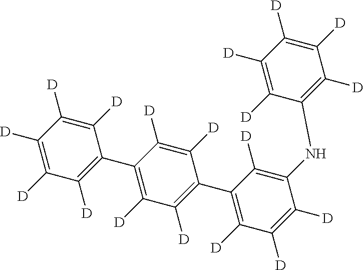 [SMILES: [2H]c1c([2H])c([2H])c(Nc2c([2H])c([2H])c([2H])c(-c3c([2H])c([2H])c(-c4c([2H])c([2H])c([2H])c([2H])c4[2H])c([2H])c3[2H])c2[2H])c([2H])c1[2H]] | 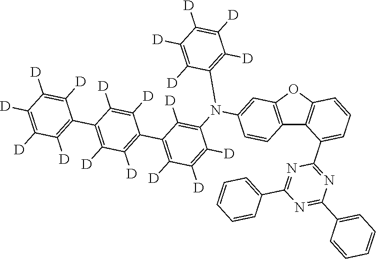 [SMILES: [2H]c1c([2H])c([2H])c(-c2c([2H])c([2H])c(-c3c([2H])c([2H])c([2H])c(N(c4ccc5c(c4)oc4cccc(-c6nc(-c7ccccc7)nc(-c7ccccc7)n6)c45)c4c([2H])c([2H])c([2H])c([2H])c4[2H])c3[2H])c([2H])c2[2H])c([2H])c1[2H]] | 70% |
| 346 | 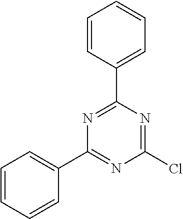 [SMILES: Clc1nc(-c2ccccc2)nc(-c2ccccc2)n1] | 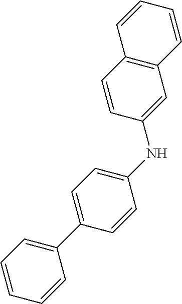 [SMILES: c1ccc(-c2ccc(Nc3ccc4ccccc4c3)cc2)cc1] | 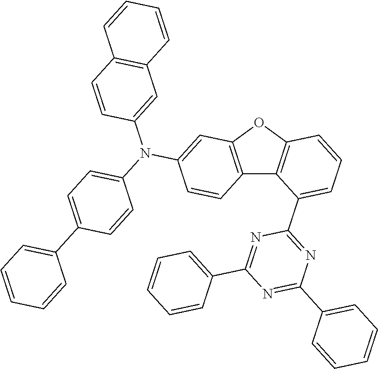 [SMILES: c1ccc(-c2ccc(N(c3ccc4ccccc4c3)c3ccc4c(c3)oc3cccc(-c5nc(-c6ccccc6)nc(-c6ccccc6)n5)c34)cc2)cc1] | 65% |
| 353 | 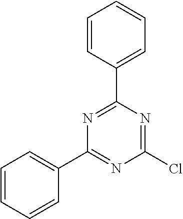 [SMILES: Clc1nc(-c2ccccc2)nc(-c2ccccc2)n1] | 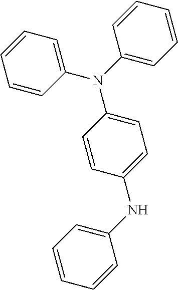 [SMILES: c1ccc(Nc2ccc(N(c3ccccc3)c3ccccc3)cc2)cc1] | 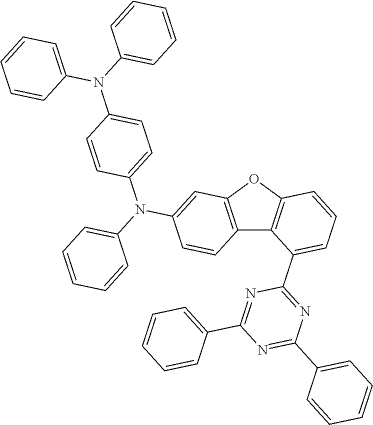 [SMILES: c1ccc(-c2nc(-c3ccccc3)nc(-c3cccc4oc5cc(N(c6ccccc6)c6ccc(N(c7ccccc7)c7ccccc7)cc6)ccc5c34)n2)cc1] | 59% |
| 361 | 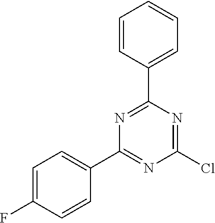 [SMILES: Fc1ccc(-c2nc(Cl)nc(-c3ccccc3)n2)cc1] | 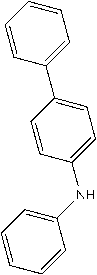 [SMILES: c1ccc(Nc2ccc(-c3ccccc3)cc2)cc1] | 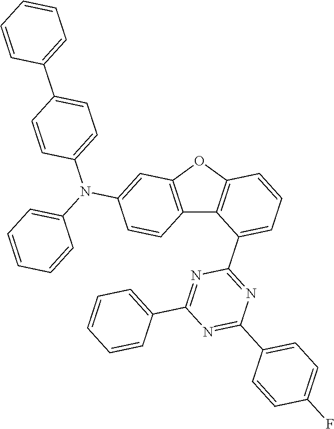 [SMILES: Fc1ccc(-c2nc(-c3ccccc3)nc(-c3cccc4oc5cc(N(c6ccccc6)c6ccc(-c7ccccc7)cc6)ccc5c34)n2)cc1] | 55% |
| 378 | 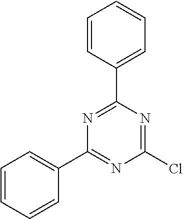 [SMILES: Clc1nc(-c2ccccc2)nc(-c2ccccc2)n1] | 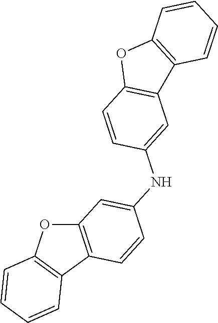 [SMILES: c1ccc2c(c1)oc1cc(Nc3ccc4oc5ccccc5c4c3)ccc12] | 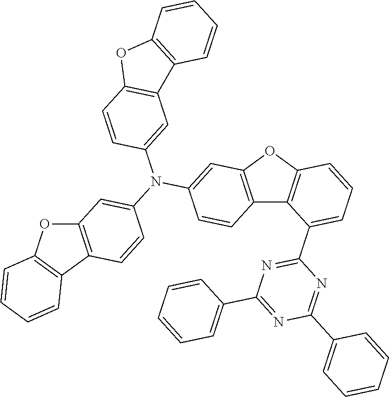 [SMILES: c1ccc(-c2nc(-c3ccccc3)nc(-c3cccc4oc5cc(N(c6ccc7c(c6)oc6ccccc67)c6ccc7oc8ccccc8c7c6)ccc5c34)n2)cc1] | 66% |
| 399 | 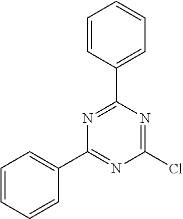 [SMILES: Clc1nc(-c2ccccc2)nc(-c2ccccc2)n1] | 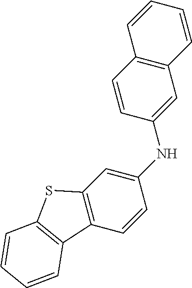 [SMILES: c1ccc2cc(Nc3ccc4c(c3)sc3ccccc34)ccc2c1] | 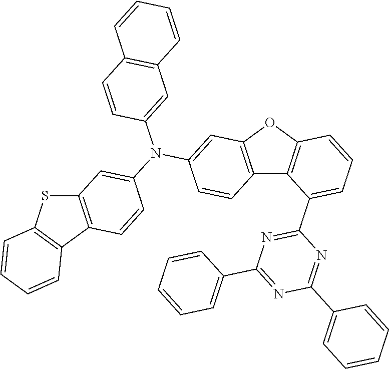 [SMILES: c1ccc(-c2nc(-c3ccccc3)nc(-c3cccc4oc5cc(N(c6ccc7ccccc7c6)c6ccc7c(c6)sc6ccccc67)ccc5c34)n2)cc1] | 55% |
| 401 | 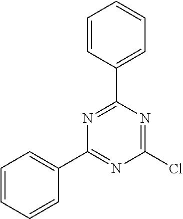 [SMILES: Clc1nc(-c2ccccc2)nc(-c2ccccc2)n1] | 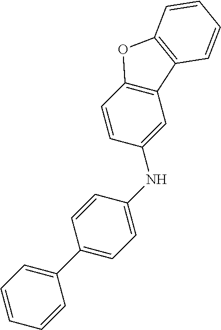 [SMILES: c1ccc(-c2ccc(Nc3ccc4oc5ccccc5c4c3)cc2)cc1] | 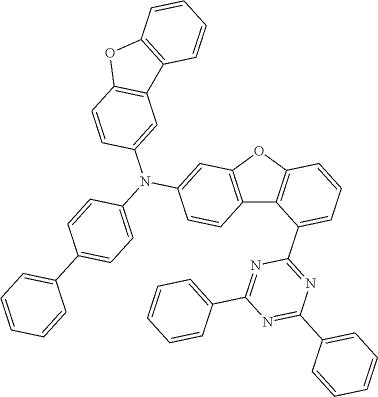 [SMILES: c1ccc(-c2ccc(N(c3ccc4c(c3)oc3cccc(-c5nc(-c6ccccc6)nc(-c6ccccc6)n5)c34)c3ccc4oc5ccccc5c4c3)cc2)cc1] | 45% |
| 421 | 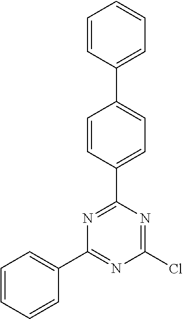 [SMILES: Clc1nc(-c2ccccc2)nc(-c2ccc(-c3ccccc3)cc2)n1] | 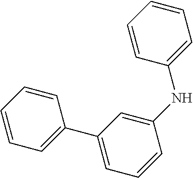 [SMILES: c1ccc(Nc2cccc(-c3ccccc3)c2)cc1] | 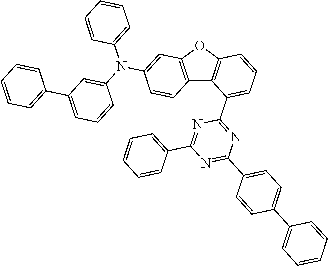 [SMILES: c1ccc(-c2ccc(-c3nc(-c4ccccc4)nc(-c4cccc5oc6cc(N(c7ccccc7)c7cccc(-c8ccccc8)c7)ccc6c45)n3)cc2)cc1] | 59% |
| 428 | 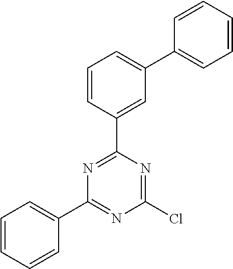 [SMILES: Clc1nc(-c2ccccc2)nc(-c2cccc(-c3ccccc3)c2)n1] | 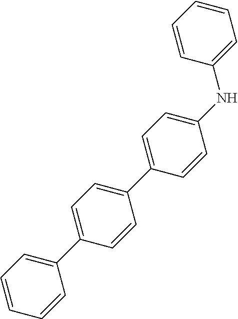 [SMILES: c1ccc(Nc2ccc(-c3ccc(-c4ccccc4)cc3)cc2)cc1] | 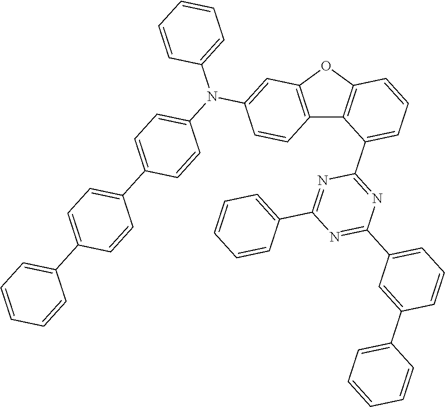 [SMILES: c1ccc(-c2ccc(-c3ccc(N(c4ccccc4)c4ccc5c(c4)oc4cccc(-c6nc(-c7ccccc7)nc(-c7cccc(-c8ccccc8)c7)n6)c45)cc3)cc2)cc1] | 66% |
| 440 | 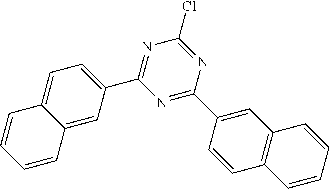 [SMILES: Clc1nc(-c2ccc3ccccc3c2)nc(-c2ccc3ccccc3c2)n1] | 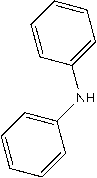 [SMILES: c1ccc(Nc2ccccc2)cc1] | 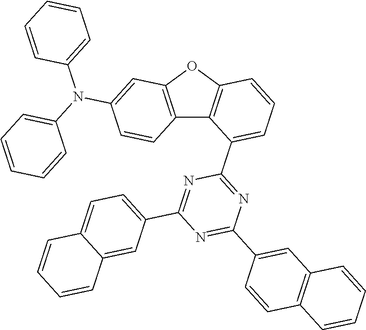 [SMILES: c1ccc(N(c2ccccc2)c2ccc3c(c2)oc2cccc(-c4nc(-c5ccc6ccccc6c5)nc(-c5ccc6ccccc6c5)n4)c23)cc1] | 57% |
| 460 | 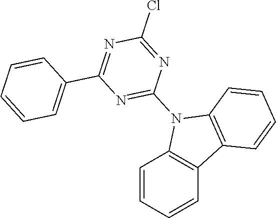 [SMILES: Clc1nc(-c2ccccc2)nc(-n2c3ccccc3c3ccccc32)n1] | 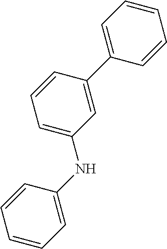 [SMILES: c1ccc(Nc2cccc(-c3ccccc3)c2)cc1] | 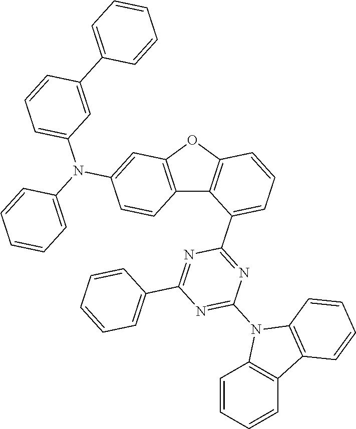 [SMILES: c1ccc(-c2cccc(N(c3ccccc3)c3ccc4c(c3)oc3cccc(-c5nc(-c6ccccc6)nc(-n6c7ccccc7c7ccccc76)n5)c34)c2)cc1] | 62% |
| 494 | 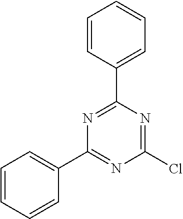 [SMILES: Clc1nc(-c2ccccc2)nc(-c2ccccc2)n1] | 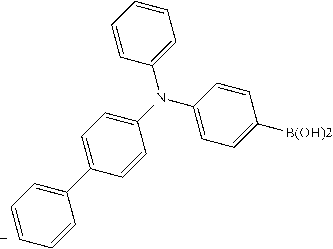 [SMILES: OB(O)c1ccc(N(c2ccccc2)c2ccc(-c3cc[c-]cc3)cc2)cc1] | 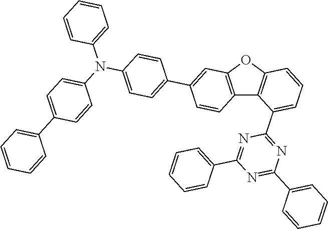 [SMILES: c1ccc(-c2ccc(N(c3ccccc3)c3ccc(-c4ccc5c(c4)oc4cccc(-c6nc(-c7ccccc7)nc(-c7ccccc7)n6)c45)cc3)cc2)cc1] | 45% |
| 501 | 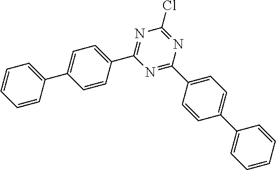 [SMILES: Clc1nc(-c2ccc(-c3ccccc3)cc2)nc(-c2ccc(-c3ccccc3)cc2)n1] | 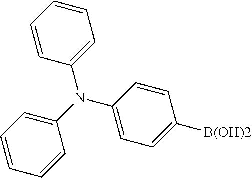 [SMILES: OB(O)c1ccc(N(c2ccccc2)c2ccccc2)cc1] | 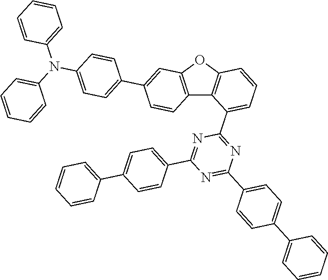 [SMILES: c1ccc(-c2ccc(-c3nc(-c4ccc(-c5ccccc5)cc4)nc(-c4cccc5oc6cc(-c7ccc(N(c8ccccc8)c8ccccc8)cc7)ccc6c45)n3)cc2)cc1] | 51% |
| 521 | 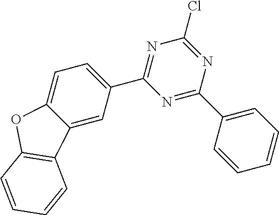 [SMILES: Clc1nc(-c2ccccc2)nc(-c2ccc3oc4ccccc4c3c2)n1] | 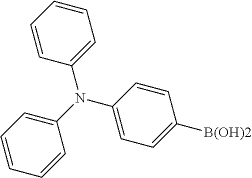 [SMILES: OB(O)c1ccc(N(c2ccccc2)c2ccccc2)cc1] | 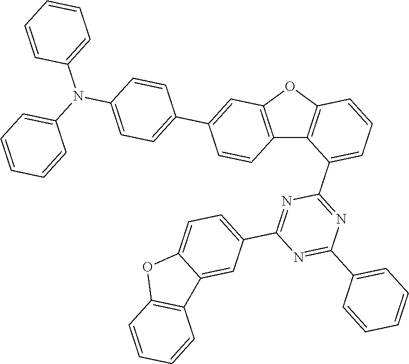 [SMILES: c1ccc(-c2nc(-c3ccc4oc5ccccc5c4c3)nc(-c3cccc4oc5cc(-c6ccc(N(c7ccccc7)c7ccccc7)cc6)ccc5c34)n2)cc1] | 59% |
| 545 | 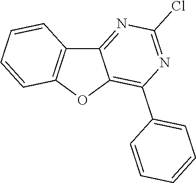 [SMILES: Clc1nc(-c2ccccc2)c2oc3ccccc3c2n1] | 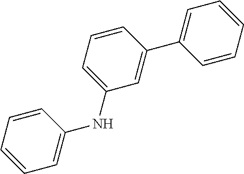 [SMILES: c1ccc(Nc2cccc(-c3ccccc3)c2)cc1] | 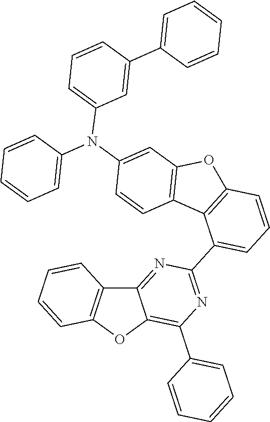 [SMILES: c1ccc(-c2cccc(N(c3ccccc3)c3ccc4c(c3)oc3cccc(-c5nc(-c6ccccc6)c6oc7ccccc7c6n5)c34)c2)cc1] | 62% |
| 565 | 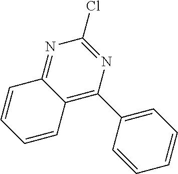 [SMILES: Clc1nc(-c2ccccc2)c2ccccc2n1] | 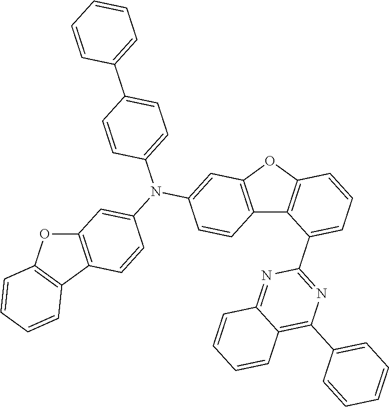 [SMILES: c1ccc(-c2ccc(N(c3ccc4c(c3)oc3ccccc34)c3ccc4c(c3)oc3cccc(-c5nc(-c6ccccc6)c6ccccc6n5)c34)cc2)cc1] | 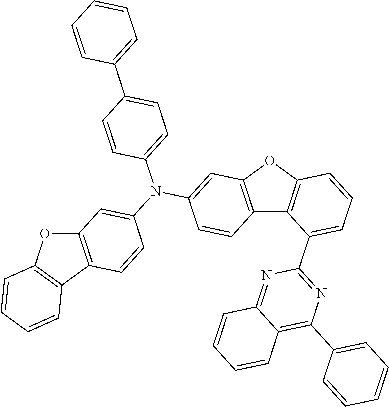 [SMILES: c1ccc(-c2ccc(N(c3ccc4c(c3)oc3ccccc34)c3ccc4c(c3)oc3cccc(-c5nc(-c6ccccc6)c6ccccc6n5)c34)cc2)cc1] | 67% |
| 571 | 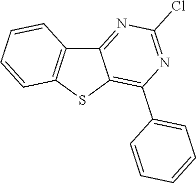 [SMILES: Clc1nc(-c2ccccc2)c2sc3ccccc3c2n1] | 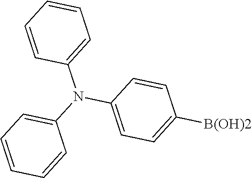 [SMILES: OB(O)c1ccc(N(c2ccccc2)c2ccccc2)cc1] | 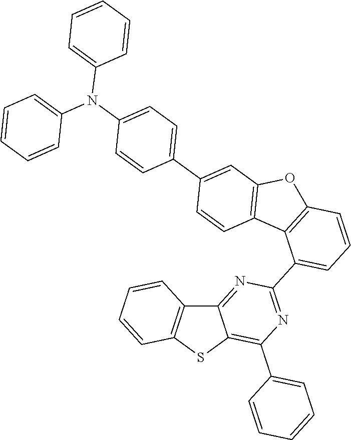 [SMILES: c1ccc(-c2nc(-c3cccc4oc5cc(-c6ccc(N(c7ccccc7)c7ccccc7)cc6)ccc5c34)nc3c2sc2ccccc23)cc1] | 72% |
| 574 | 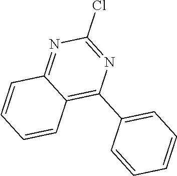 [SMILES: Clc1nc(-c2ccccc2)c2ccccc2n1] | 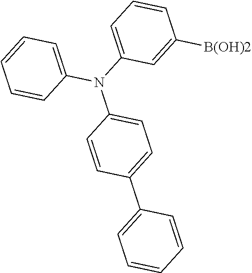 [SMILES: OB(O)c1cccc(N(c2ccccc2)c2ccc(-c3ccccc3)cc2)c1] | 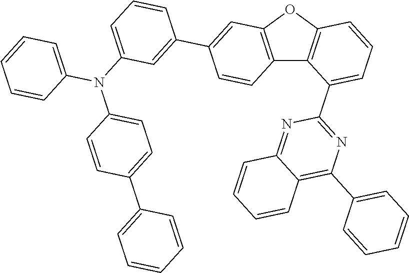 [SMILES: c1ccc(-c2ccc(N(c3ccccc3)c3cccc(-c4ccc5c(c4)oc4cccc(-c6nc(-c7ccccc7)c7ccccc7n6)c45)c3)cc2)cc1] | 68% |
| 581 | 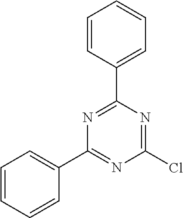 [SMILES: Clc1nc(-c2ccccc2)nc(-c2ccccc2)n1] | 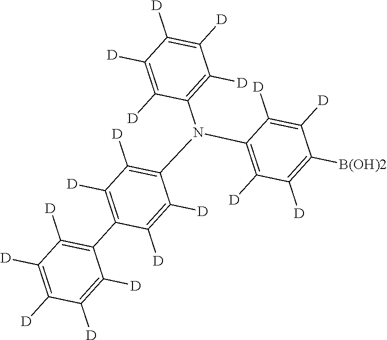 [SMILES: [2H]c1c([2H])c([2H])c(-c2c([2H])c([2H])c(N(c3c([2H])c([2H])c([2H])c([2H])c3[2H])c3c([2H])c([2H])c(B(O)O)c([2H])c3[2H])c([2H])c2[2H])c([2H])c1[2H]] | 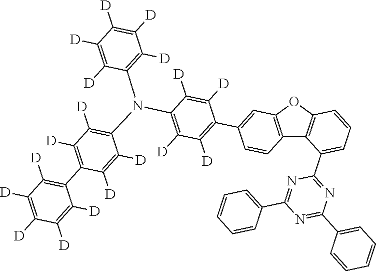 [SMILES: [2H]c1c([2H])c([2H])c(-c2c([2H])c([2H])c(N(c3c([2H])c([2H])c([2H])c([2H])c3[2H])c3c([2H])c([2H])c(-c4ccc5c(c4)oc4cccc(-c6nc(-c7ccccc7)nc(-c7ccccc7)n6)c45)c([2H])c3[2H])c([2H])c2[2H])c([2H])c1[2H]] | 59% |
| 582 | 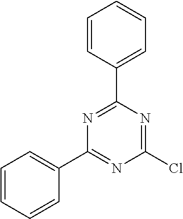 [SMILES: Clc1nc(-c2ccccc2)nc(-c2ccccc2)n1] | 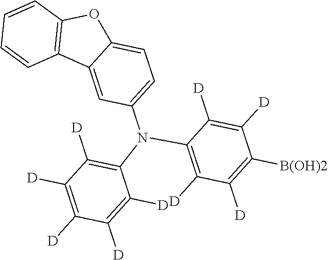 [SMILES: [2H]c1c([2H])c([2H])c(N(c2ccc3oc4ccccc4c3c2)c2c([2H])c([2H])c(B(O)O)c([2H])c2[2H])c([2H])c1[2H]] | 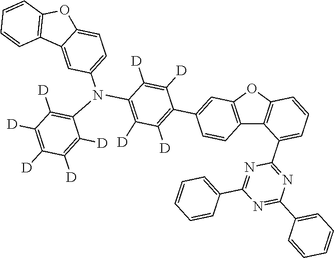 [SMILES: [2H]c1c([2H])c([2H])c(N(c2ccc3oc4ccccc4c3c2)c2c([2H])c([2H])c(-c3ccc4c(c3)oc3cccc(-c5nc(-c6ccccc6)nc(-c6ccccc6)n5)c34)c([2H])c2[2H])c([2H])c1[2H]] | 74% |
| 340 | 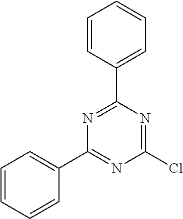 [SMILES: Clc1nc(-c2ccccc2)nc(-c2ccccc2)n1] | 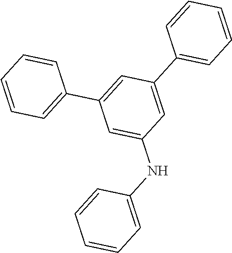 [SMILES: c1ccc(Nc2cc(-c3ccccc3)cc(-c3ccccc3)c2)cc1] | 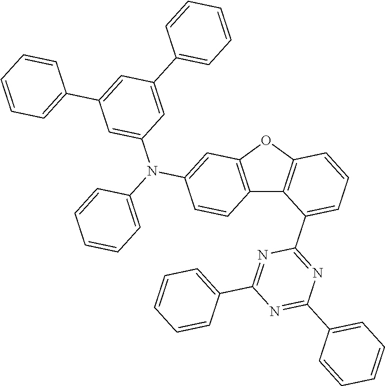 [SMILES: c1ccc(-c2cc(-c3ccccc3)cc(N(c3ccccc3)c3ccc4c(c3)oc3cccc(-c5nc(-c6ccccc6)nc(-c6ccccc6)n5)c34)c2)cc1] | 67% |
| 410 | 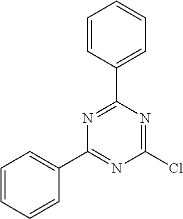 [SMILES: Clc1nc(-c2ccccc2)nc(-c2ccccc2)n1] | 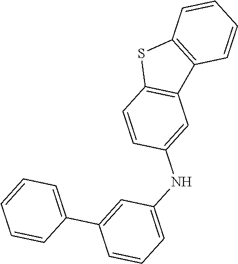 [SMILES: c1ccc(-c2cccc(Nc3ccc4sc5ccccc5c4c3)c2)cc1] | 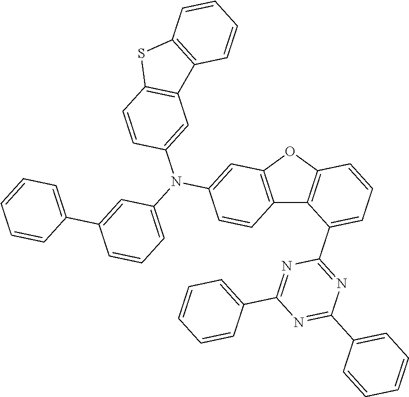 [SMILES: c1ccc(-c2cccc(N(c3ccc4c(c3)oc3cccc(-c5nc(-c6ccccc6)nc(-c6ccccc6)n5)c34)c3ccc4sc5ccccc5c4c3)c2)cc1] | 77% |
| 422 | 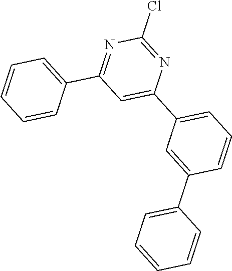 [SMILES: Clc1nc(-c2ccccc2)cc(-c2cccc(-c3ccccc3)c2)n1] | 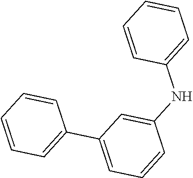 [SMILES: c1ccc(Nc2cccc(-c3ccccc3)c2)cc1] | 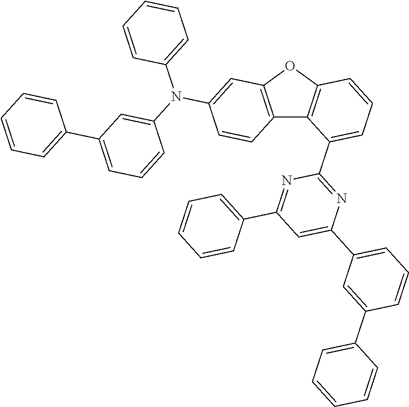 [SMILES: c1ccc(-c2cccc(-c3cc(-c4ccccc4)nc(-c4cccc5oc6cc(N(c7ccccc7)c7cccc(-c8ccccc8)c7)ccc6c45)n3)c2)cc1] | 69% |
| 520 | 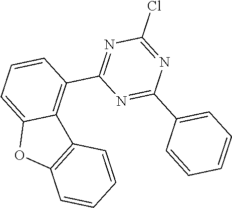 [SMILES: Clc1nc(-c2ccccc2)nc(-c2cccc3oc4ccccc4c23)n1] | 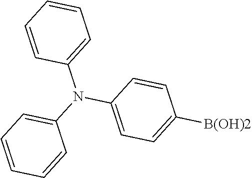 [SMILES: OB(O)c1ccc(N(c2ccccc2)c2ccccc2)cc1] | 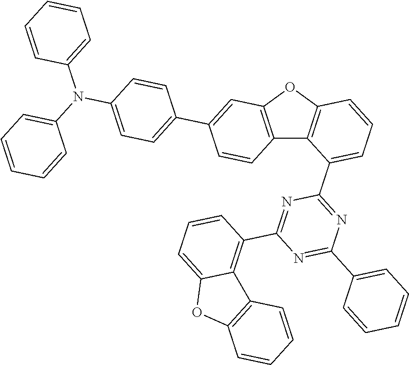 [SMILES: c1ccc(-c2nc(-c3cccc4oc5ccccc5c34)nc(-c3cccc4oc5cc(-c6ccc(N(c7ccccc7)c7ccccc7)cc6)ccc5c34)n2)cc1] | 58% |
| 549 | 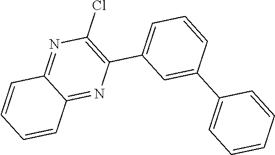 [SMILES: Clc1nc2ccccc2nc1-c1cccc(-c2ccccc2)c1] | 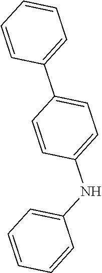 [SMILES: c1ccc(Nc2ccc(-c3ccccc3)cc2)cc1] | 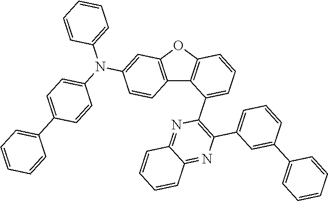 [SMILES: c1ccc(-c2ccc(N(c3ccccc3)c3ccc4c(c3)oc3cccc(-c5nc6ccccc6nc5-c5cccc(-c6ccccc6)c5)c34)cc2)cc1] | 68% |
| 580 | 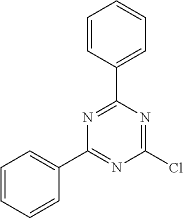 [SMILES: Clc1nc(-c2ccccc2)nc(-c2ccccc2)n1] | 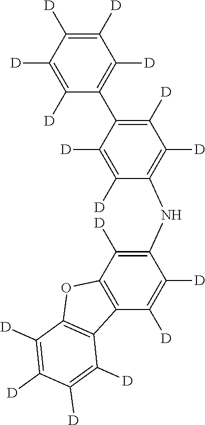 [SMILES: [2H]c1c([2H])c([2H])c(-c2c([2H])c([2H])c(Nc3c([2H])c([2H])c4c(oc5c([2H])c([2H])c([2H])c([2H])c54)c3[2H])c([2H])c2[2H])c([2H])c1[2H]] | 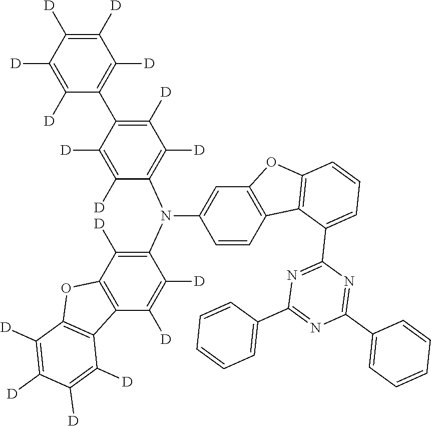 [SMILES: [2H]c1c([2H])c([2H])c(-c2c([2H])c([2H])c(N(c3ccc4c(c3)oc3cccc(-c5nc(-c6ccccc6)nc(-c6ccccc6)n5)c34)c3c([2H])c([2H])c4c(oc5c([2H])c([2H])c([2H])c([2H])c54)c3[2H])c([2H])c2[2H])c([2H])c1[2H]] | 77% |
| 582 | 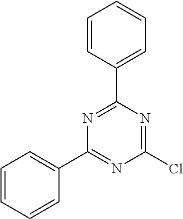 [SMILES: Clc1nc(-c2ccccc2)nc(-c2ccccc2)n1] | 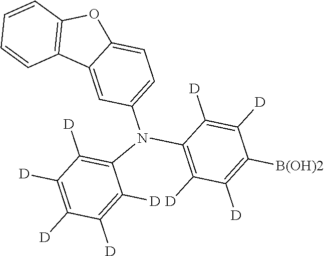 [SMILES: [2H]c1c([2H])c([2H])c(N(c2ccc3oc4ccccc4c3c2)c2c([2H])c([2H])c(B(O)O)c([2H])c2[2H])c([2H])c1[2H]] | 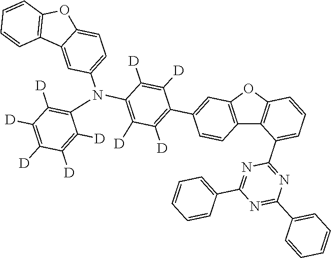 [SMILES: [2H]c1c([2H])c([2H])c(N(c2ccc3oc4ccccc4c3c2)c2c([2H])c([2H])c(-c3ccc4c(c3)oc3cccc(-c5nc(-c6ccccc6)nc(-c6ccccc6)n5)c34)c([2H])c2[2H])c([2H])c1[2H]] | 75% |
| 584 | 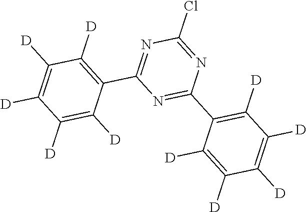 [SMILES: [2H]c1c([2H])c([2H])c(-c2nc(Cl)nc(-c3c([2H])c([2H])c([2H])c([2H])c3[2H])n2)c([2H])c1[2H]] | 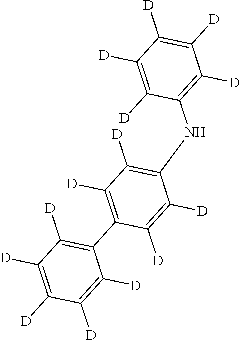 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] | 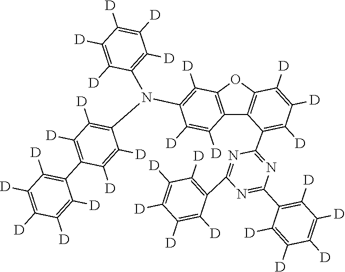 [SMILES: [2H]c1c([2H])c([2H])c(-c2nc(-c3c([2H])c([2H])c([2H])c([2H])c3[2H])nc(-c3c([2H])c([2H])c([2H])c4oc5c([2H])c(N(c6c([2H])c([2H])c([2H])c([2H])c6[2H])c6c([2H])c([2H])c(-c7c([2H])c([2H])c([2H])c([2H])c7[2H])c([2H])c6[2H])c([2H])c([2H])c5c34)n2)c([2H])c1[2H]] | 74% |
| 589 | 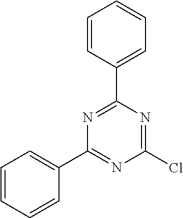 [SMILES: Clc1nc(-c2ccccc2)nc(-c2ccccc2)n1] | 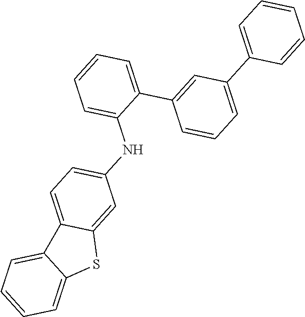 [SMILES: c1ccc(-c2cccc(-c3ccccc3Nc3ccc4c(c3)sc3ccccc34)c2)cc1] | 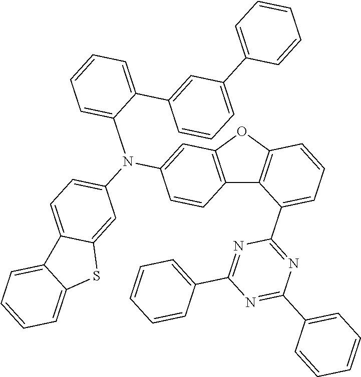 [SMILES: c1ccc(-c2cccc(-c3ccccc3N(c3ccc4c(c3)oc3cccc(-c5nc(-c6ccccc6)nc(-c6ccccc6)n5)c34)c3ccc4c(c3)sc3ccccc34)c2)cc1] | 63% |
| 594 | 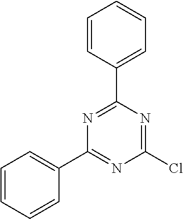 [SMILES: Clc1nc(-c2ccccc2)nc(-c2ccccc2)n1] | 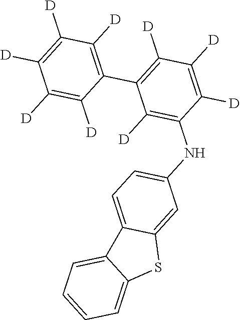 [SMILES: [2H]c1c([2H])c([2H])c(-c2c([2H])c([2H])c([2H])c(Nc3ccc4c(c3)sc3ccccc34)c2[2H])c([2H])c1[2H]] | 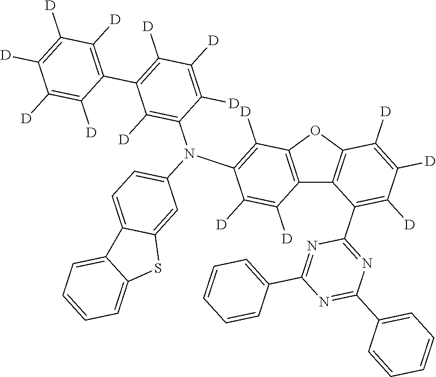 [SMILES: [2H]c1c([2H])c([2H])c(-c2c([2H])c([2H])c([2H])c(N(c3ccc4c(c3)sc3ccccc34)c3c([2H])c([2H])c4c(oc5c([2H])c([2H])c([2H])c(-c6nc(-c7ccccc7)nc(-c7ccccc7)n6)c54)c3[2H])c2[2H])c([2H])c1[2H]] | 66% |
| 595 | 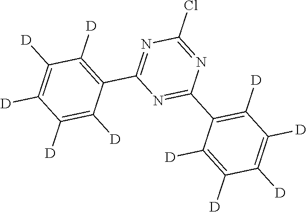 [SMILES: [2H]c1c([2H])c([2H])c(-c2nc(Cl)nc(-c3c([2H])c([2H])c([2H])c([2H])c3[2H])n2)c([2H])c1[2H]] | 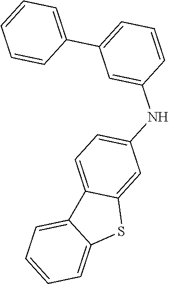 [SMILES: c1ccc(-c2cccc(Nc3ccc4c(c3)sc3ccccc34)c2)cc1] | 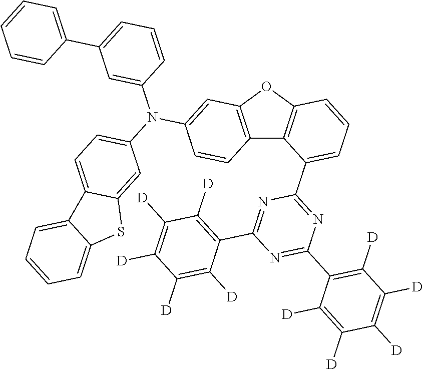 [SMILES: [2H]c1c([2H])c([2H])c(-c2nc(-c3c([2H])c([2H])c([2H])c([2H])c3[2H])nc(-c3cccc4oc5cc(N(c6cccc(-c7ccccc7)c6)c6ccc7c(c6)sc6ccccc67)ccc5c34)n2)c([2H])c1[2H]] | 69% |
| 371 | 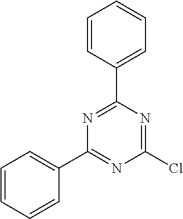 [SMILES: Clc1nc(-c2ccccc2)nc(-c2ccccc2)n1] | 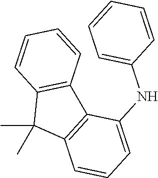 [SMILES: CC1(C)c2ccccc2-c2c(Nc3ccccc3)cccc21] | 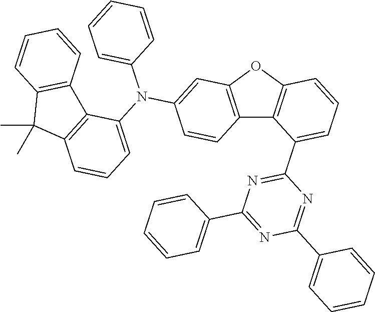 [SMILES: CC1(C)c2ccccc2-c2c(N(c3ccccc3)c3ccc4c(c3)oc3cccc(-c5nc(-c6ccccc6)nc(-c6ccccc6)n5)c34)cccc21] | 66% |
| 397 | 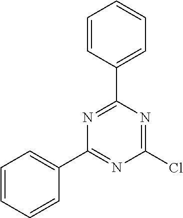 [SMILES: Clc1nc(-c2ccccc2)nc(-c2ccccc2)n1] | 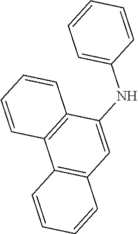 [SMILES: c1ccc(Nc2cc3ccccc3c3ccccc23)cc1] | 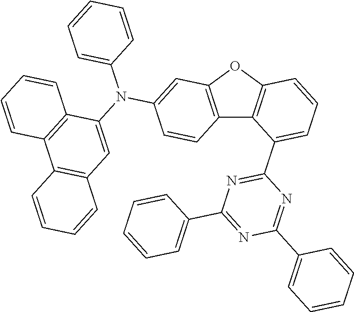 [SMILES: c1ccc(-c2nc(-c3ccccc3)nc(-c3cccc4oc5cc(N(c6ccccc6)c6cc7ccccc7c7ccccc67)ccc5c34)n2)cc1] | 67% |
| 398 | 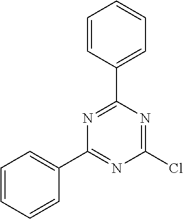 [SMILES: Clc1nc(-c2ccccc2)nc(-c2ccccc2)n1] | 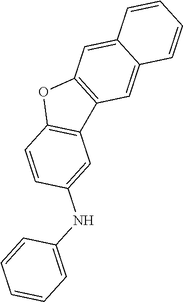 [SMILES: c1ccc(Nc2ccc3oc4cc5ccccc5cc4c3c2)cc1] | 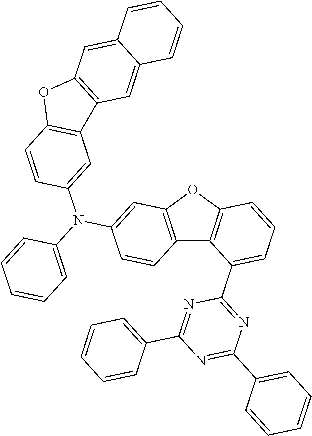 [SMILES: c1ccc(-c2nc(-c3ccccc3)nc(-c3cccc4oc5cc(N(c6ccccc6)c6ccc7oc8cc9ccccc9cc8c7c6)ccc5c34)n2)cc1] | 70% |
| 596 | 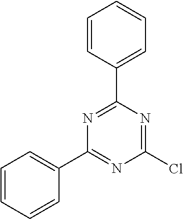 [SMILES: Clc1nc(-c2ccccc2)nc(-c2ccccc2)n1] | 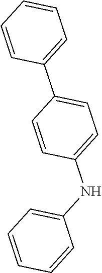 [SMILES: c1ccc(Nc2ccc(-c3ccccc3)cc2)cc1] | 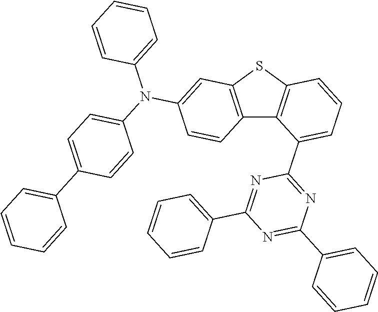 [SMILES: c1ccc(-c2ccc(N(c3ccccc3)c3ccc4c(c3)sc3cccc(-c5nc(-c6ccccc6)nc(-c6ccccc6)n5)c34)cc2)cc1] | 68% |
| 600 | 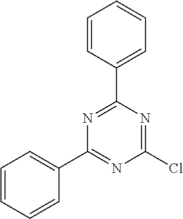 [SMILES: Clc1nc(-c2ccccc2)nc(-c2ccccc2)n1] | 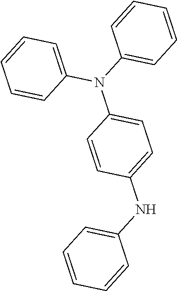 [SMILES: c1ccc(Nc2ccc(N(c3ccccc3)c3ccccc3)cc2)cc1] | 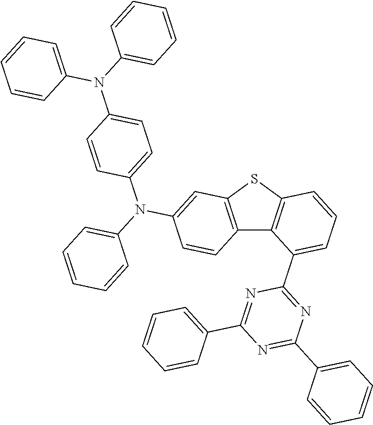 [SMILES: c1ccc(-c2nc(-c3ccccc3)nc(-c3cccc4sc5cc(N(c6ccccc6)c6ccc(N(c7ccccc7)c7ccccc7)cc6)ccc5c34)n2)cc1] | 56% |
| 601 | 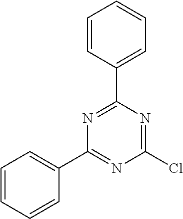 [SMILES: Clc1nc(-c2ccccc2)nc(-c2ccccc2)n1] | 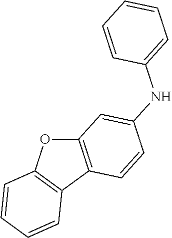 [SMILES: c1ccc(Nc2ccc3c(c2)oc2ccccc23)cc1] | 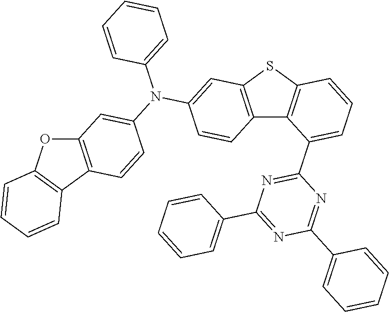 [SMILES: c1ccc(-c2nc(-c3ccccc3)nc(-c3cccc4sc5cc(N(c6ccccc6)c6ccc7c(c6)oc6ccccc67)ccc5c34)n2)cc1] | 61% |
| 604 | 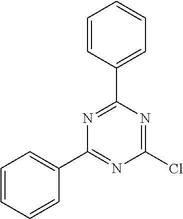 [SMILES: Clc1nc(-c2ccccc2)nc(-c2ccccc2)n1] | 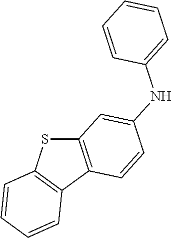 [SMILES: c1ccc(Nc2ccc3c(c2)sc2ccccc23)cc1] | 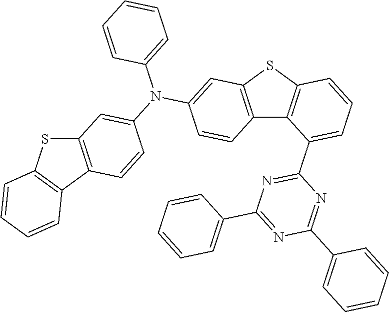 [SMILES: c1ccc(-c2nc(-c3ccccc3)nc(-c3cccc4sc5cc(N(c6ccccc6)c6ccc7c(c6)sc6ccccc67)ccc5c34)n2)cc1] | 70% |
| 612 | 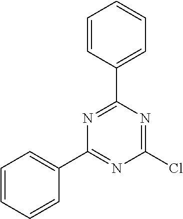 [SMILES: Clc1nc(-c2ccccc2)nc(-c2ccccc2)n1] | 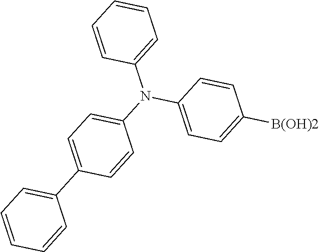 [SMILES: OB(O)c1ccc(N(c2ccccc2)c2ccc(-c3ccccc3)cc2)cc1] | 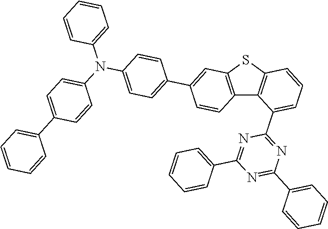 [SMILES: c1ccc(-c2ccc(N(c3ccccc3)c3ccc(-c4ccc5c(c4)sc4cccc(-c6nc(-c7ccccc7)nc(-c7ccccc7)n6)c45)cc3)cc2)cc1] | 69% |
| 617 | 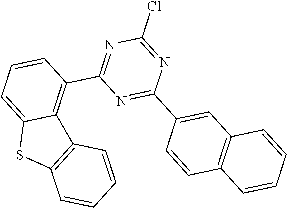 [SMILES: Clc1nc(-c2ccc3ccccc3c2)nc(-c2cccc3sc4ccccc4c23)n1] | 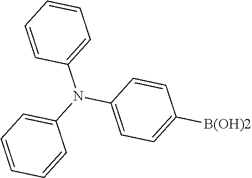 [SMILES: OB(O)c1ccc(N(c2ccccc2)c2ccccc2)cc1] | 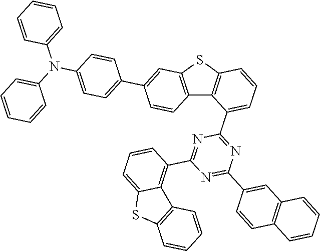 [SMILES: c1ccc(N(c2ccccc2)c2ccc(-c3ccc4c(c3)sc3cccc(-c5nc(-c6ccc7ccccc7c6)nc(-c6cccc7sc8ccccc8c67)n5)c34)cc2)cc1] | 67% |
| 619 | 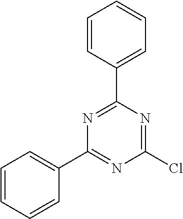 [SMILES: Clc1nc(-c2ccccc2)nc(-c2ccccc2)n1] | 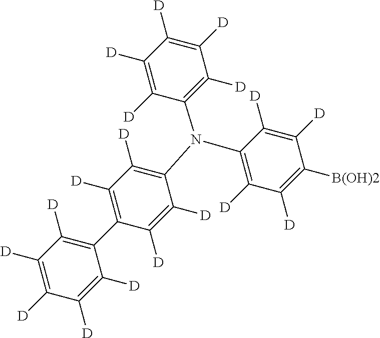 [SMILES: [2H]c1c([2H])c([2H])c(-c2c([2H])c([2H])c(N(c3c([2H])c([2H])c([2H])c([2H])c3[2H])c3c([2H])c([2H])c(B(O)O)c([2H])c3[2H])c([2H])c2[2H])c([2H])c1[2H]] | 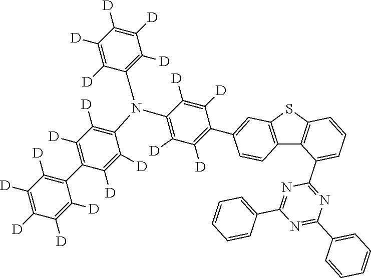 [SMILES: [2H]c1c([2H])c([2H])c(-c2c([2H])c([2H])c(N(c3c([2H])c([2H])c([2H])c([2H])c3[2H])c3c([2H])c([2H])c(-c4ccc5c(c4)sc4cccc(-c6nc(-c7ccccc7)nc(-c7ccccc7)n6)c45)c([2H])c3[2H])c([2H])c2[2H])c([2H])c1[2H]] | 72% |
| 620 | 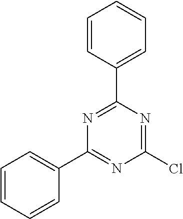 [SMILES: Clc1nc(-c2ccccc2)nc(-c2ccccc2)n1] | 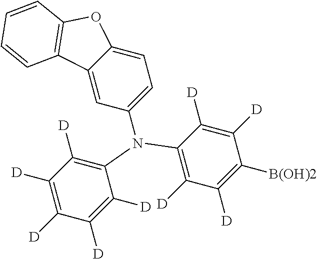 [SMILES: [2H]c1c([2H])c([2H])c(N(c2ccc3oc4ccccc4c3c2)c2c([2H])c([2H])c(B(O)O)c([2H])c2[2H])c([2H])c1[2H]] | 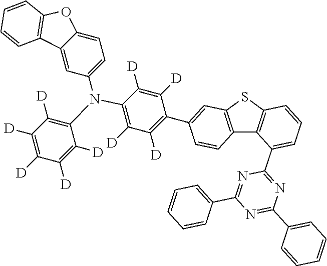 [SMILES: [2H]c1c([2H])c([2H])c(N(c2ccc3oc4ccccc4c3c2)c2c([2H])c([2H])c(-c3ccc4c(c3)sc3cccc(-c5nc(-c6ccccc6)nc(-c6ccccc6)n5)c34)c([2H])c2[2H])c([2H])c1[2H]] | |

TABLE 4

| NO | [symbol] H NMR(CDCl₃, 300 Mz) |
|---|---|
| 12 | 8.36 (d, 4H), 7.97 (d, 1H), 7.83 (s, 2H), 7.78 (d, 1H), 7.76 (s, 1H), 7.69 (d, 2H), 7.63 |
| | (d, 2H), 7.57 (d, 1H), 7.54 (d, 1H), 7.50 (t, 6H), 7.39 (t, 1H), 7.31 (t, 1H) |
| 28 | 8.36 (d, 4H), 8.09 (d, 1H), 8.06 (d, 1H), 7.99 (d, 1H), 7.83 (s, 1H), 7.78 (d, 1H), 7.76 |
| | (s, 1H), 7.63 (t, 1H), 7.58 (t, 1H), 7.55 (s, 1H), 7.50 (t, 6H), 7.28 (d, 1H), 7.25 (s, 4H) |
| 51 | 8.36 (d, 2H), 8.25 (d, 2H), 7.83 (s, 1H), 7.78 (d, 1H), 7.76 (s, 1H), 7.75 (d, 4H), 7.69- |
| | 7.41 (m, 9H), 7.25 (d, 6H) |
| 75 | 8.55 (d, 1H), 8.36 (d, 2H), 7.94 (s, 1H), 7.89 (s, 1H), 7.78-7.41 (m, 25H), 7.16 (t, 1H), |
| | 7.11 (t, 1H) |
| 96 | 8.36 (d, 2H), 8.25 (d, 2H), 8.08 (d, 1H), 7.98 (d, 1H), 7.83 (s, 1H), 7.78 (d, 1H), 7.76 |
| | (s, 1H), 7.75 (d, 2H), 7.69-7.31 (m, 14H), 7.25 (d, 6H) |
| 154 | 8.95 (d, 1H), 8.50 (d, 1H), 8.36 (d, 2H), 8.20 (s, 1H), 8.12 (s, 1H), 8.12 (d, 1H), 8.04 |
| | (s, 3H), 7.99 (d, 1H), 7.97 (d, 1H), 7.89 (d, 1H), 7.78-7.77 (d, 2H), 7.75 (d, 4H), 7.67 |
| | (d, 1H), 7.50 (t, 3H), 7.49-7.35 (m, 8H) |
| 176 | 8.36 (d, 4H), 8.18 (s, 1H), 7.90 (d, 1H), 7.78-7.68 (m, 6H), 7.57 (d, 2H), 7.55 (d, 1H), |
| | 7.50 (t, 6H), 7.38 (t, 1H), 7.28 (t, 1H), 1.69 (s, 6H) |
| 216 | 9.15 (s, 1H), 8.51 (d, 1H), 8.36 (d, 2H), 7.99 (d, 1H), 7.91 (d, 1H), 7.78 (d, 2H), 7.76 |
| | (s, 2H), 7.75 (d, 4H), 7.57-7.41 (m, 13H) |
| 226 | 8.36 (d, 2H), 8.09 (d, 1H), 8.06 (d, 1H), 7.99 (d, 1H), 7.96 (d, 1H), 7.78 (d, 2H), 7.76 |
| | (s, 2H), 7.63-7.31 (m, 15H) |
| 240 | 8.38 (d, 1H), 8.36 (d, 2H), 8.09 (d, 1H), 8.06 (d, 1H), 7.99 (d, 1H), 7.94 (s, 1H), 7.78 |
| | (d, 2H), 7.76 (s, 2H), 7.75 (d, 2H), 7.63-7.38 (m, 13H), 7.25 (dd, 4H) |
| 252 | 9.02 (d, 1H), 8.95 (d, 1H), 8.36 (d, 4H), 8.09 (d, 1H), 8.06 (d, 1H), 7.99 (d, 1H), 7.78 |
| | (d, 3H), 7.76 (s, 2H), 7.63-7.35 (m, 15H) |
| 280 | 8.95 (d, 1H), 8.50 (d, 1H), 8.36 (d, 4H), 8.20 (s, 2H), 8.17 (d, 1H), 7.97 (d, 2H), 7.89 |
| | (d, 1H), 7.78 (d, 1H), 7.77 (d, 1H), 7.67 (d, 1H), 7.50 (t, 6H), 7.41 (t, 1H), 7.35 (t, |
| | 1H), 7.25 (d, 4H) |
| 318 | 9.08 (4, 1H), 8.84 (d, 1H), 8.36 (d, 4H), 8.27 (d, 1H), 8.25 (d, 2H), 8.20 (s, 2H), 8.17 |
| | (d, 2H), 7.97 (d, 2H), 7.90 (d, 1H), 7.70-7.63 (m, 4H), 7.50 (t, 6H), 7.25 (d, 2H) |
| 326 | 8.25 (d, 2H), 8.09 (d, 1H), 8.06 (d, 1H), 7.99 (d, 1H), 7.78 (d, 2H), 7.76 (s, 2H), 7.63 |
| | (t, 1H), 7.58 (t, 1H), 7.57 (d, 2H), 7.55 (s, 1H), 7.38 (d, 1H), 7.25 (d, 2H) |
| 330 | 7.83 (s, 1H), 7.78 (d, 1H), 7.76 (s, 1H), 7.69 (d, 1H), 7.63 (d, 1H), 7.57 (d, 1H) |
| 338 | 8.36 (d, 4H), 8.10 (d, 1H), 8.03 (s, 1H), 7.75 (d, 2H), 7.60-7.34 (m, 22H), 7.14 (t, |
| | 1H), 7.08 (d, 2H), 6.91 (d, 1H) |
| 346 | 8.36 (d, 4H), 8.03 (s, 1H), 7.75 (d, 2H), 7.71 (d, 1H), 7.62-7.38 (m, 20H), 7.27 (d, |
| | 2H), 7.11 (s, 1H), 6.91 (d, 1H) |
| 353 | 8.36 (d, 4H), 8.03 (s, 1H), 7.60 (d, 1H), 7.57 (d, 1H), 7.55 (d, 1H), 7.50 (t, 6H), 7.47 |
| | (t, 1H), 7.24 (t, 6H), 7.08-7.00 (m, 13H), 6.91 (d, 1H) |
| 378 | 8.36 (d, 4H), 8.22 (s, 1H), 8.03 (s, 2H), 7.98 (d, 2H), 7.60-7.31 (m, 18H), 6.97 (d, |
| | 1H), 6.91 (d, 2H) |
| 401 | 8.36 (d, 4H), 8.22 (s, 1H), 8.03 (s, 1H), 7.98 (d, 1H), 7.75 (d, 2H), 7.60 (d, 1H), 7.57- |
| | 7.27 (m, 20H), 6.97 (d, 1H), 6.91 (d, 1H) |
| 460 | 8.55 (d, 1H), 8.36 (d, 2H), 8.19 (d, 1H), 8.03 (s, 1H), 7.75 (d, 2H), 7.60 (d, 1H), 7.57 |
| | (d, 1H), 7.55 (d, 1H), 7.55 (t, 1H), 7.50-7.24 (m, 9H), 7.27-7.08 (m, 12H), 6.91 (d, |
| | 1H) |
| 494 | 8.36 (d, 4H), 7.78 (d, 1H), 7.76 (d, 1H), 7.75 (d, 2H), 7.60-7.41 (m, 17H), 7.27 (d, |
| | 4H), 7.24 (t, 2H), 7.08 (d, 2H), 7.00 (t, 1H) |
| 545 | 8.03 (s, 1H), 7.84 (d, 2H), 7.75 (d, 2H), 7.70 (d, 1H), 7.65 (d, 1H), 7.60 (d.1H), 7.57 |
| | (d, 1H), 7.55 (d, 1H), 7.55 (t, 1H), 7.53 (t, 2H), 7.49 (t, 3H), 7.41 (t, 1H), 7.36 (t, 1H), |
| | 7.27-7.17 (m, 6H), 7.08 (d, 2H), 7.00 (t, 1H), 6.91 (d, 1H) |
| 571 | 7.86 (d, 1H), 7.84 (d, 2H), 7.78 (d, 2H), 7.76 (s, 1H), 7.60 (d, 1H), 7.57 (d, 2H), 7.55 |
| | (d, 2H), 7.53 (t, 2H), 7.49 (t, 1H), 7.47 (t, 1H), 7.33-7.24 (m, 8H), 7.08 (d, 4H), 7.00 |
| | (t, 2H) |
| 574 | 8.13 (d, 1H), 7.84 (d, 1H), 7.83 (t, 1H), 7.80 (d, 2H), 7.78 (d, 1H), 7.76 (s, 1H), 7.75 |
| | (d, 2H), 7.65-7.41 (m, 14H), 7.27 (d, 2H), 7.27 (s, 1H), 7.24 (t, 2H), 7.18 (d, 1H), |
| | 7.17 (d, 1H), 7.08 (d, 2H), 7.00 (t, 1H) |
| 581 | 8.36 (d, 4H), 7.78 (d, 1H), 7.76 (s, 1H), 7.60 (d, 1H), 7.57 (d, 2H), 7.50 (t, 6H), 7.47 |
| | (t, 1H) |
| 582 | 8.36 (d, 4H), 8.22 (s, 1H), 7.98 (d, 1H), 7.78 (d, 1H), 7.76 (s, 1H), 7.60 (d, 1H), 7.57 |
| | (d, 2H), 7.54 (d, 1H), 7.50 (t, 6H), 7.47 (t, 1H), 7.46 (d, 1H), 7.39 (t, 1H), 7.31 (t, 1H), |
| | 6.97 (d, 1H) |
| 4 | 9.08 (d, 1H), 8.84 (d, 1H), 8.36 (d, 4H), 8.27 (d, 1H), 8.05 (s, 1H), 7.90 (d, 1H), 7.78- |
| | 7.50 (m, 16H) |
| 22 | 8.36 (d, 4H), 8.19 (d, 1H), 7.83 (s, 1H), 7.79 (d, 1H), 7.78 (d, 1H), 7.76 (s, 1H), 7.69 |
| | (d, 1H), 7.63 (d, 1H), 7.62 (m, 3H), 7.58 (t, 1H), 7.57 (d, 1H), 7.50 (m, 8H), 7.40 (d, |
| | 1H), 7.24-7.18 (m, 3H) |
| 410 | 8.45 (d, 1H), 8.46 (d, 4H), 8.03 (s, 1H), 7.95 (s, 1H), 7.86 (d, 2H), 7.75 (d, 2H), 7.60 |
| | (d, 1H), 7.57 (d, 1H), 7.56 (t, 1H), 7.55 (t, 1H), 7.55 (d, 1H), 7.50 (t, 5H), 7.49 (t, |
| | 2H), 7.47 (t, 1H), 7.41 (t, 1H), 7.31 (t, 1H), 7.27 (s, 1H), 7.17 (d, 2H), 6.94 (d, 1H) |
| 422 | 8.23 (s, 1H), 8.03 (s, 1H), 7.94 (d, 2H), 7.75 (d, 4H), 7.71 (t, 1H), 7.60-7.41 (m, 15H), |
| | 7.27 (s, 1H), 7.24 (t, 2H), 7.17 (d, 2H), 7.08 (d, 2H), 7.00 (t, 1H), 6.91 (d, 1H) |
| 520 | 8.36 (d, 2H), 7.98 (d, 1H), 7.78 (d, 1H), 7.76 (s, 1H), 7.60-7.24 (m, 21H), 7.08 (d, |
| | 4H), 7.00 (t, 2H) |
| 549 | 8.50 (s, 1H), 8.50 (d, 1H), 8.13 (d, 1H), 8.03 (s, 1H), 7.80 (d, 2H), 7.75 (d, 5H), 7.71 |
| | (d, 1H), 7.70 (d, 1H), 7.67 (t, 2H), 7.55 (d, 3H), 7.51 (t, 1H), 7.49 (t, 4H), 7.41 (t, 2H), |
| | 7.27 (d, 2H), 7.24 (t, 2H), 7.08 (d, 2H), 7.00 (t, 1H), 6.91 (d, 1H) |
| 589 | 8.45 (d, 1H), 8.36 (d, 4H), 8.10 (d, 1H), 8.03 (s, 1H), 7.94 (s, 1H), 7.86 (d, 1H), 7.75 |
| | (d, 2H), 7.74 (d, 1H), 7.71 (t, 1H), 7.61-7.31 (m, 21H), 7.14 (t, 1H), 6.91 (d, 1H) |
| 268 | 8.45(d, 1H), 8.36(d, 4H), 8.20 (s, 2H), 8.17 (d, 1H), 7.97(d, 2H), 7.96 (d, 1H), 7.86 (d, |
| | 1H), 7.67 (d, 2H), 7.56 (t, 1H), 7.50 (t, 7H), 7. [symbol] 1 (t, 1H) |
| 44 | 8.55(d, 2H), 8.36 (d, 4H), 8.20 (d, 2H), 7.92 (d, 2H), 7.83 (s, 1H), 7.78 (d, 1H), 7.76 |
| | (s, 1H), 7.69-7.50 (m, 17H), 7.16-7.08 (m, 5H) |
| 47 | 8.36 (d, 4H), 8.20 (d, 2H), 8.19 (d, 1H), 7.98 (d, 1H), 7.92 (d, 2H), 7.83 (s, 1H), 7.78 |
| | (d, 1H), 7.76 (s, 1H), 7.69 (d, 1H), 7.63 (d, 1H), 7.54(d, 1H), 7.50 (t, 6H), 7.49 (s, |
| | 1H), 7.42 (s, 1H), 7.40 (d, 1H), 7.39 (t, 1H), 7.20 (t, 2H) |
| 371 | 8.36 (d, 4H), 8.03 (s, 1H), 7.90 (d, 1H), 7.60-7.50 (m, 12H), 7.24-7.21 (m, 6H), 7.08- |
| | 7.00 (d, 3H), 6.98 (d [symbol] 1H) |
| 397 | 8.98 (d, 1H), 8.84 (d, 1H), 8.36 (d, 4H), 8.11 (d, 1H), 8.03 (s, 1H), 7.90 (d, 1H), 7. [symbol] 8- |
| | 7.50 (m, 15H), 7.24 (t 2H), 7.08 (d, 2H), 7.00 (t, 1H), 6.91 (d, 1H) |
| 398 | 8.36 (d, 4H), 8.28 (d, 1H), 8.22 (s, 1H), 8.03 (s, 1H), 7.84(d, 1H), 7.75 (t, 1H), 7.60- |
| | 7.42 (m, 15H), 7.24 (t, 2H), 7.08-6.97(m, 5H) |
| 596 | 8.36 (d, 4H), 7.96 (d, 1H), 7.67 (d, 1H), 7.74 (d, 1H), 7.64 (s, 1H), 7.55 (d, 2H), 7.50 |
| | (t, 8H), 7.49 (t, 2H), 7.43 (d, 1H), 7.27 (d, 2H), 7.24 (t, 2H), 7.08 (d, 2H), 7.00 (t, 1H) |
| 600 | 8.36 (d, 4H), 7.96 (s, 1H), 2.74 (d, 1H), 7.62 (d, 1H), 7.64 (d, 1H), 7.50 (t, 6H), 7.47 |
| | (t, 1H), 7.24 (t, 6H), 7.08-7.00 (m, 13H) |
| 601 | 8.36 (d, 4H), 8.22(s, 1H), 7.98 (d, 1H), 7.96 (d, 1H), 7.74 (d, 1H), 7.67 (d, 1H), 7.64 |
| | (s, 1H), 7.54 (d, 1H), 7.50 (t, 7H), 7.46 (d, 1H), 7.43 (d, 1H), 7.39 (t, 1H), 7.31 (t, 1H), |
| | 7.24 (t, 1H), 7.08-6.97 (m, 4H) |
| 604 | 8.45 (d, 1H), 8.36 (d, 4H), 7.96 (d, 1H), 7.86 (d, 1H), 7.74-7.64 (d, 5H), 7.56 (t, 1H), |
| | 7.50 (t, 7H), 7.43 (d, 1H), 7.31 (t, 1H), 7.24 (t, 2H), 7.08 (d, 2H), 7.00 (t, 1H) |
| 612 | 8.36 (d, 4H), 8.20 (s, 1H), 8.17 (d, 1H), 7.97 (d, 2H), 7.75 (d, 2H), 7.55 (d, 4H), 7.50 |
| | (t, 7H), 7.49 (t, 2H), 7.41 (t, 1H), 7.27 (d, 4H), 7.24 (t, 2H), 7.08 (d, 2H), 7.00 (t, 1H) |
| 617 | 9.09 (s, 1H), 8.49 (d, 2H), 8.20 (s, 1H), 8.17 (d, 2H), 8.08 (d, 1H), 8.00 (d, 1H), 7.96 |
| | (d, 3H), 7.86 (d, 1H), 7.61-7.50 (m, 9H), 7.31 (t, 1H), 7.27 (d, 2H), 7.24 (t, 4H), 7.08 |
| | (d, 4H) 7.00 (t, 2H). |

[symbol]

indicates data missing or illegible when filed

TABLE 5

| Compound | FD-MS | Compound | FD-MS |
|---|---|---|---|
| 12 | m/z = 565.18 (C [symbol] O₂ = 565.63) | 28 | m/z = 601.22 (C₄ [symbol] O = 601.71) |
| 51 | m/z = 627.23 (C₄ [symbol] O = 627.75) | 75 | m/z = 716.84 (C [symbol] N₄O = 716.26) |
| 96 | m/z = 717.24 (C [symbol] N₃O₂ = 717.83) | 154 | m/z = 693.22 (C [symbol] S = 693.87) |
| 176 | m/z = 591.23 (C₄ [symbol] O = 591.71) | 216 | m/z = 601.22 (C [symbol] O = 601.71) |
| 226 | m/z = 615.19 (C₄ [symbol] O₂ = 615.69) | 240 | m/z = 677.25 (C [symbol] O = 677.81) |
| 252 | m/z = 651.23 (C [symbol] O = 651.77) | 280 | m/z = 617.19 (C [symbol] S = 617.77) |
| 318 | m/z = 667.21 (C₄ [symbol] S = 667.83) | 326 | m/z = 691.33(C [symbol] O = 691.89) |
| 330 | m/z = 622.35 (C₄ [symbol] O = 622.84) | 338 | m/z = 718.27 (C₅ [symbol] ₄N₄O = 718.86) |
| 346 | m/z = 692.26 (C₄ [symbol] N₄O = 692.82) | 353 | m/z = 733.28 (C₅₁H [symbol] N₅O = 733.88) |
| 378 | m/z = 746.23 (C [symbol] = 746.83) | 401 | m/z = 732.25 (C [symbol] N₄O₂ = 732.84) |
| 460 | m/z = 731.27 (C₅₁H [symbol] O = 731.86) | 494 | m/z = 718.27 (C₅₁H [symbol] ₄N₄O = 718.86) |
| 545 | m/z = 655.23 (C [symbol] O₂ = 655.76) | 571 | m/z = 671.20 (C₄ [symbol] OS = 671.82) |
| 574 | m/z = 691.26 (C₅ [symbol] O = 691.83) | 581 | m/z = 736.39(C₅ [symbol] N₄O = 736.97) |
| 582 | m/z = 741.31(C₅ [symbol] N₄O₂ = 741.90) | 4 | m/z = 575.20 (C₄ [symbol] O = 575.67) |
| 12 | m/z = 565.18 (C [symbol] O₂ = 565.63) | 22 | m/z = 640.23 (C₄₅H₂₈N₄O = 640.75) |
| 323 | m/z = 673.25 (C₄ [symbol] S = 673.87) | 651 | m/z = 628.38 (C₄ [symbol] H₂₇N₅O = 628.87) |
| 652 | m/z = 748.44 (C₅ [symbol] O₂ = 749.02) | 340 | m/z = 718.27 (C₅ [symbol] O = 718.86) |
| 410 | m/z = 748.23 (C [symbol] OS = 748.90) | 422 | m/z = 717.28 (C [symbol] O = 717.87) |
| 520 | m/z = 732.25 (C [symbol] ₂N₄O₂ = 732.84) | 549 | m/z = 691.26 (C [symbol] O = 691.83) |
| 580 | m/z = 748.35(C [symbol] N₄O₂ = 748.94) | 584 | m/z = 672.43 (C [symbol] N₄O = 672.95) |
| 589 | m/z = 824.26 (C₅₇ [symbol] N₄OS = 825.00) | 594 | m/z = 763.32(C₅ [symbol] ₅N₄OS = 764.00) |
| 268 | m/z = 597.13 (C [symbol] S₂ = 597.75) | 595 | m/z = 758.29(C₅ [symbol] N₄OS = 758.97) |
| 44 | m/z = 805.28 (C [symbol] O = 805.94) | 47 | m/z = 730.24 (C [symbol] N₄O₂ = 730.83) |
| 371 | m/z = 682.27 (C₄ [symbol] N₄O = 682.83) | 397 | m/z = 666.24 (C [symbol] N₄O = 666.78) |
| 398 | m/z = 706.24 (C [symbol] O₂ = 706.81) | 596 | m/z = 658.22 (C [symbol] N₄S = 658.82) |
| 600 | m/z = 749.26 (C₅ [symbol] S = 749.94) | 601 | m/z = 672.20 (C₄₅H₂ [symbol] N₄OS = 672.81) |
| 604 | m/z = 688.18 (C [symbol] S₂ = 688.87) | 612 | m/z = 734.25 (C [symbol] ₄N₄S = 734.92) |
| 617 | m/z = 814.22 (C [symbol] S₂ = 815.03) | 619 | m/z = 752.36(C [symbol] N₄S = 753.03) |
| 620 | m/z = 757.29 (=757.96) | | |

[symbol]

indicates data missing or illegible when filed

### Experimental Example 1

(1) Manufacture of Organic Light Emitting Device (Red Single Host & Mix Host)

A glass substrate, in which ITO/Ag/ITO were thinly coated to have a thickness of 115 Å/100 Å/15 Å, was ultrasonically washed with distilled water. When the washing with distilled water is finished, the glass substrate was ultrasonically washed with a solvent such as acetone, methanol, and isopropyl alcohol, was dried and then was subjected to UVO treatment for 5 minutes by using UV in a UV washing machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition. As the common layers, a hole injection layer HAT-CN, a hole transport layer α-NPB, a hole transport auxiliary layer TPD(N-([1,1′-biphenyl]-4-yl)-N,1′-diphenyl-1′H-spiro[fluorene-9,5′-naphtho[8,1,2,3-cdef]carbazol]-7′-amine) and an electron blocking layer TAPC(N-([1,1′-biphenyl]-4-yl)-N,1′-diphenyl-1′H-spiro[fluorene-9,5′-naphtho[8,1,2,3-cdef]carbazol]-7′-amine) were formed to have a thickness of 100 Å, 1100 Å, 800 Å and 150 Å, respectively, on the ITO electrode (positive electrode). A light emitting layer was thermally vacuum deposited thereon as follows.

The light emitting layer was deposited to have a thickness of 400 Å by depositing two types of the compounds described in the following Table 7 as a red host from a single supply source and doping the host with (piq)₂ (Ir) (acac) at 3 wt % using (piq)₂ (Ir) (acac) as a red phosphorescent dopant. Thereafter, Bphen as a hole blocking layer was deposited to have a thickness of 30 Å, and TPBI as an electron transport layer was deposited to have a thickness of 250 Å thereon. Finally, lithium fluoride (LiF) was deposited to have a thickness of 10 Å on the electron transport layer to form an electron injection layer, and then a silver (Ag) negative electrode was deposited to have a thickness of 200 Å on the electron injection layer to form a negative electrode, thereby manufacturing an organic electroluminescence device. Meanwhile, all the organic compounds required for manufacturing an OLED device were subjected to vacuum sublimed purification under 10⁻⁸ to 10⁻⁶ torr for each material, and used for the manufacture of OLED.

In this case, Comparative Compounds A to I used in Comparative Examples 1 to 9 and 36 to 42 are as follows.

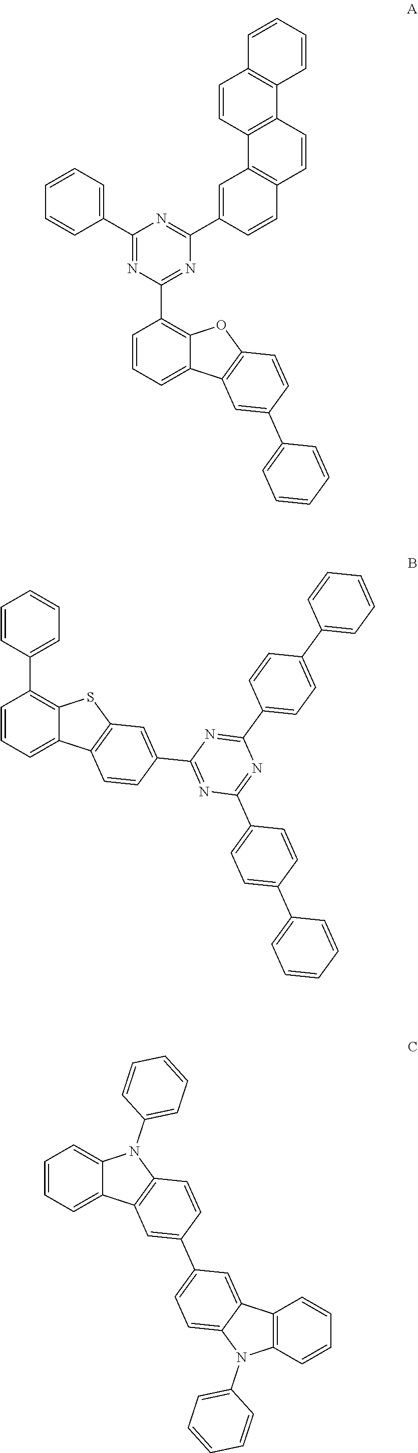 [SMILES: c1ccc(-c2ccc3oc4c(-c5nc(-c6ccccc6)nc(-c6ccc7ccc8c9ccccc9ccc8c7c6)n5)cccc4c3c2)cc1 ; c1ccc(-c2ccc(-c3nc(-c4ccc(-c5ccccc5)cc4)nc(-c4ccc5c(c4)sc4c(-c6ccccc6)cccc45)n3)cc2)cc1 ; c1ccc(-n2c3ccccc3c3cc(-c4ccc5c(c4)c4ccccc4n5-c4ccccc4)ccc32)cc1]

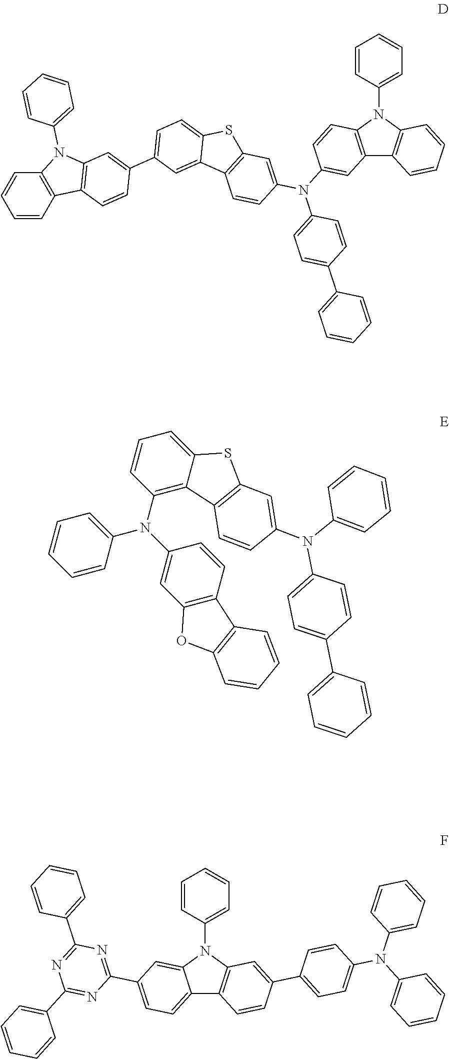 [SMILES: c1ccc(-c2ccc(N(c3ccc4c(c3)sc3ccc(-c5ccc6c7ccccc7n(-c7ccccc7)c6c5)cc34)c3ccc4c(c3)c3ccccc3n4-c3ccccc3)cc2)cc1 ; c1ccc(-c2ccc(N(c3ccccc3)c3ccc4c(c3)sc3cccc(N(c5ccccc5)c5ccc6c(c5)oc5ccccc56)c34)cc2)cc1 ; c1ccc(-c2nc(-c3ccccc3)nc(-c3ccc4c5ccc(-c6ccc(N(c7ccccc7)c7ccccc7)cc6)cc5n(-c5ccccc5)c4c3)n2)cc1]

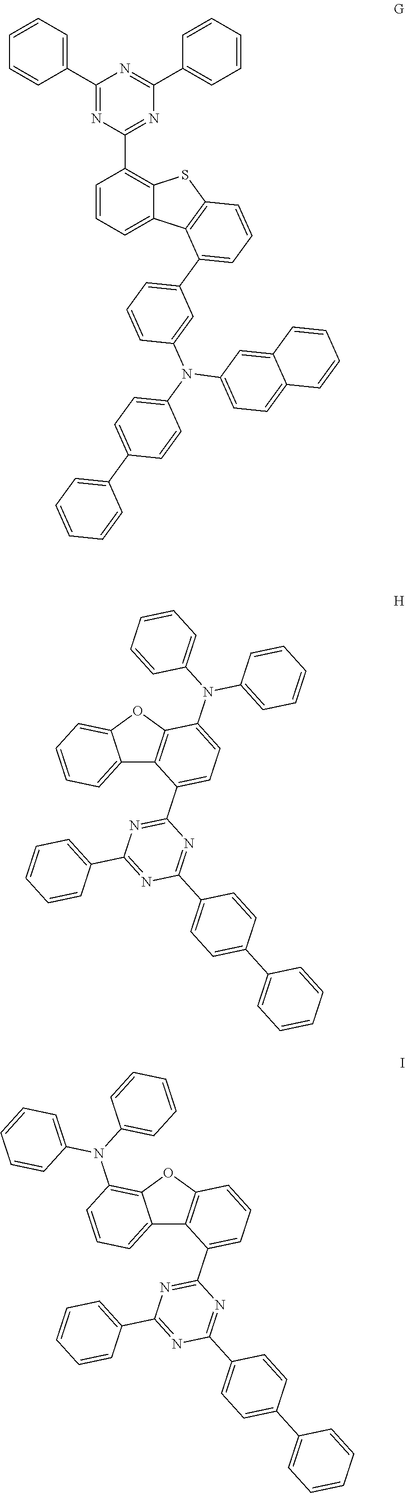 [SMILES: c1ccc(-c2ccc(N(c3cccc(-c4cccc5sc6c(-c7nc(-c8ccccc8)nc(-c8ccccc8)n7)cccc6c45)c3)c3ccc4ccccc4c3)cc2)cc1 ; c1ccc(-c2ccc(-c3nc(-c4ccccc4)nc(-c4ccc(N(c5ccccc5)c5ccccc5)c5oc6ccccc6c45)n3)cc2)cc1 ; c1ccc(-c2ccc(-c3nc(-c4ccccc4)nc(-c4cccc5oc6c(N(c7ccccc7)c7ccccc7)cccc6c45)n3)cc2)cc1]

(2) Driving Voltage and Light Emitting Efficiency of Organic Electroluminescence Device

For the organic electroluminescence device manufactured as described above, electroluminescence (EL) characteristics were measured by M7000 manufactured by McScience Inc., and based on the measurement result thereof, T95 was measured by a service life measurement device (M6000) manufactured by McScience Inc., when the reference luminance was 6,000 cd/m². As described above, T₉₅ means the service life (unit: hour) of the device measured at the time when the luminance reaches 95% relative to the initial luminance.

The following Table 6 shows an example of applying a single host material, and Table 7 shows, as an example of depositing two host compounds as one supply source, an experimental example of using the compounds of Chemical Formulae 1 and 2 of the present invention as the host materials of the light emitting layer or a comparative example of using only any one of Chemical Formulae 1 and 2 of the present invention.

TABLE 6

| | | Driving | | Color | Service |
|---|---|---|---|---|---|
| | | voltage | Efficiency | coordinate | life |
| | Compound | (V) | (cd/A) | (x, y) | (T95) |
| Comparative | A | 4.80 | 19.8 | (0.682, | 12 |
| example 1 | | | | 0.317) | |
| Comparative | B | 4.78 | 18.8 | (0.682, | 15 |
| example 2 | | | | 0.317) | |
| Comparative | C | 4.87 | 12.6 | (0.681, | 32 |
| example 3 | | | | 0.319) | |
| Comparative | D | 4.77 | 10.6 | (0.681, | 21 |
| example 4 | | | | 0.319) | |
| Comparative | E | 4.69 | 10.8 | (0.681, | 19 |
| example 5 | | | | 0.319) | |
| Comparative | F | 4.62 | 10.2 | (0.681, | 10 |
| example 6 | | | | 0.319) | |
| Comparative | G | 4.52 | 12.4 | (0.682, | 28 |
| example 7 | | | | 0.317) | |
| Comparative | H | 4.58 | 14 | (0.681, | 35 |
| example 8 | | | | 0.319) | |
| Comparative | I | 4.60 | 15.4 | (0.682, | 38 |
| example 9 | | | | 0.317) | |
| Comparative | 12 | 4.49 | 24.8 | (0.682, | 35 |
| example 10 | | | | 0.317) | |
| Comparative | 28 | 4.40 | 27.2 | (0.682, | 40 |
| example 11 | | | | 0.317) | |
| Comparative | 51 | 4.40 | 26.4 | (0.682, | 39 |
| example 12 | | | | 0.317) | |
| Comparative | 75 | 4.35 | 28.4 | (0.682, | 37 |
| example 13 | | | | 0.317) | |
| Comparative | 96 | 4.38 | 29.6 | (0.682, | 42 |
| example 14 | | | | 0.317) | |
| Comparative | 154 | 4.44 | 20.4 | (0.682, | 45 |
| example 15 | | | | 0.317) | |
| Comparative | 176 | 4.33 | 23.8 | (0.681, | 38 |
| example 16 | | | | 0.319) | |
| Comparative | 216 | 4.39 | 29.0 | (0.682, | 41 |
| example 17 | | | | 0.317) | |
| Comparative | 226 | 4.37 | 27.8 | (0.684, | 42 |
| example 18 | | | | 0.316) | |
| Comparative | 240 | 4.36 | 28.8 | (0.682, | 40 |
| example 19 | | | | 0.317) | |
| Comparative | 252 | 4.35 | 30 | (0.682, | 44 |
| example 20 | | | | 0.317) | |
| Comparative | 280 | 4.33 | 24.2 | (0.682, | 39 |
| example 21 | | | | 0.317) | |
| Comparative | 318 | 4.30 | 23.4 | (0.682, | 37 |
| example 22 | | | | 0.317) | |
| Comparative | 326 | 4.37 | 29.6 | (0.681, | 52 |
| example 23 | | | | 0.319) | |
| Comparative | 330 | 4.40 | 27.4 | (0.681, | 53 |
| example 24 | | | | 0.319) | |
| Comparative | 338 | 4.24 | 28.4 | (0.684, | 68 |
| example 25 | | | | 0.316) | |
| Comparative | 346 | 4.30 | 29.5 | (0.682, | 64 |
| example 26 | | | | 0.317) | |
| Comparative | 353 | 4.20 | 26.9 | (0.681, | 62 |
| example 27 | | | | 0.319) | |
| Comparative | 378 | 4.22 | 27.9 | (0.681, | 70 |
| example 28 | | | | 0.319) | |
| Comparative | 401 | 4.31 | 31.4 | (0.682, | 75 |
| example 29 | | | | 0.317) | |
| Comparative | 460 | 4.37 | 27.4 | (0.682, | 62 |
| example 30 | | | | 0.317) | |
| Comparative | 494 | 4.34 | 32.6 | (0.682, | 80 |
| example 31 | | | | 0.317) | |
| Comparative | 545 | 4.26 | 28.8 | (0.682, | 72 |
| example 32 | | | | 0.317) | |
| Comparative | 571 | 4.29 | 31.1 | (0.682, | 67 |
| example 33 | | | | 0.317) | |
| Comparative | 574 | 4.22 | 26.6 | (0.681, | 70 |
| example 34 | | | | 0.319) | |
| Comparative | 581 | 4.34 | 33.5 | (0.681, | 95 |
| example 35 | | | | 0.319) | |

TABLE 7

| | | | | | | Color | |
|---|---|---|---|---|---|---|---|
| | | | | Driving | Effi- | coor- | Service |
| | First | Second | Ratio | voltage | ciency | dinate | life |
| | host | host | (N:P) | (V) | (cd/A) | (x, y) | (T95) |
| Com- | A | 401 | 1:1 | 4.22 | 32.2 | (0.682, | 72 |
| parative | | | | | | 0.317) | |
| example 36 | | | | | | | |
| Example 1 | 240 | | | 3.60 | 59.9 | (0.682, | 135 |
| | | | | | | 0.317) | |
| Com- | 252 | E | | 4.92 | 42 | (0.681, | 78 |
| parative | | | | | | 0.319) | |
| example 37 | | | | | | | |
| Example 2 | | 494 | | 3.55 | 62.3 | (0.681, | 138 |
| | | | | | | 0.319) | |
| Com- | 96 | I | 1:2 | 4.15 | 44 | (0.681, | 97 |
| parative | | | | | | 0.319) | |
| example 38 | | | | | | | |
| Example 3 | | 346 | | 3.70 | 58.3 | (0.682, | 128 |
| | | | | | | 0.317) | |
| Example 4 | 216 | 581 | 1:3 | 3.54 | 65.3 | (0.682, | 164 |
| | | | | | | 0.317) | |
| Com- | | D | | 4.24 | 28.5 | (0.682, | 79 |
| parative | | | | | | 0.317) | |
| example 39 | | | | | | | |
| Example 5 | 226 | 571 | | 3.65 | 55.7 | (0.682, | 127 |
| | | | | | | 0.317) | |
| Example 6 | 176 | 378 | | 3.58 | 52.3 | (0.681, | 130 |
| | | | | | | 0.319) | |
| Example 7 | 326 | 581 | 1:2 | 3.54 | 68.8 | (0.681, | 170 |
| | | | | | | 0.319) | |
| Com- | 28 | C | | 3.78 | 50.0 | (0.681, | 80 |
| parative | | | | | | 0.319) | |
| example 40 | | | | | | | |
| Com- | 318 | F | | 4.24 | 38.6 | (0.681, | 70 |
| parative | | | | | | 0.319) | |
| example 41 | | | | | | | |
| Example 8 | 51 | 338 | 1:1 | 3.66 | 61.2 | (0.681, | 133 |
| | | | | | | 0.319) | |
| Example 9 | 280 | | | 3.75 | 56.8 | (0.682, | 125 |
| | | | | | | 0.317) | |
| Com- | B | 353 | | 3.99 | 28.9 | (0.682, | 72 |
| parative | | | | | | 0.317) | |
| example 42 | | | | | | | |
| Example 10 | 154 | | | 3.79 | 49.9 | (0.682, | 122 |
| | | | | | | 0.317) | |
| Example 11 | 12 | 460 | 2:1 | 3.50 | 58.7 | (0.681, | 124 |
| | | | | | | 0.319) | |
| Example 12 | 75 | 545 | 2:1 | 3.91 | 58 | (0.681, | 132 |
| | | | | | | 0.319) | |
| Example 13 | 330 | 582 | 1:3 | 3.62 | 66.1 | (0.682, | 160 |
| | | | | | | 0.317) | |
| Example 14 | 323 | 338 | 1:1 | 3.78 | 55.9 | (0.682, | 120 |
| | | | | | | 0.317) | |
| Com- | 651 | C | 1:2 | 3.92 | 51.0 | (0.681, | 81 |
| parative | | | | | | 0.319) | |
| example 43 | | | | | | | |
| Example 16 | 4 | 460 | 1:1 | 3.60 | 54.4 | (0.681, | 121 |
| | | | | | | 0.319) | |
| Example 17 | 22 | 571 | 2:1 | 3.88 | 56 | (0.682, | 127 |
| | | | | | | 0.317) | |
| Example 18 | 652 | 332 | 2:1 | 3.64 | 60.1 | (0.682, | 151 |
| | | | | | | 0.317) | |
| Example 19 | 654 | 581 | 1:1 | 3.54 | 68.8 | (0.681, | 174 |
| | | | | | | 0.319) | |
| Example 20 | 660 | 580 | 1:1 | 3.58 | 60.3 | (0.682, | 158 |
| | | | | | | 0.317) | |
| Example 21 | 298 | 410 | 1:2 | 3.73 | 58.7 | (0.682, | 136 |
| | | | | | | 0.317) | |
| Example 22 | 51 | 422 | 1:1 | 3.76 | 59.2 | (0.681, | 100 |
| | | | | | | 0.319) | |
| Example 23 | 318 | 549 | 1:1 | 3.85 | 54.8 | (0.682, | 98 |
| | | | | | | 0.317) | |
| Example 24 | 154 | 340 | 1:1 | 3.70 | 50.9 | (0.682, | 105 |
| | | | | | | 0.317) | |
| Example 25 | 330 | 589 | 1:3 | 3.60 | 68.1 | (0.682, | 123 |
| | | | | | | 0.317) | |
| Example 26 | 84 | 594 | 1:2 | 3.63 | 60.7 | (0.682, | 136 |
| | | | | | | 0.317) | |
| Example 27 | 55 | 584 | 1:1 | 3.64 | 62.2 | (0.681, | 107 |
| | | | | | | 0.319) | |
| Example 28 | 69 | 520 | 1:2 | 3.65 | 62.8 | (0.681, | 116 |
| | | | | | | 0.319) | |
| Example 29 | 84 | 595 | 1:2 | 3.63 | 60.7 | (0.682, | 122 |
| | | | | | | 0.317) | |
| Example 30 | 318 | 548 | 1:1 | 3.82 | 54.9 | (0.682, | 98 |
| | | | | | | 0.317) | |
| Example 31 | 268 | 378 | 1:3 | 3.64 | 51.3 | (0.681, | 100 |
| | | | | | | 0.319) | |
| Example 32 | 44 | 571 | 1:1 | 3.88 | 55.9 | (0.682, | 96 |
| | | | | | | 0.317) | |
| Example 33 | 47 | 545 | 1:1 | 3.88 | 54.9 | (0.682, | 98 |
| | | | | | | 0.317) | |
| Example 34 | 280 | 371 | 1:2 | 3.64 | 51.0 | (0.681, | 103 |
| | | | | | | 0.319) | |
| Example 35 | 96 | 397 | 1:2 | 3.70 | 56.3 | (0.682, | 110 |
| | | | | | | 0.317) | |
| Example 36 | 240 | 398 | 1:1 | 3.60 | 58.9 | (0.682, | 114 |
| | | | | | | 0.317) | |
| Example 37 | 55 | 596 | 1:2 | 3.71 | 60.2 | (0.681, | 112 |
| | | | | | | 0.319) | |
| Example 38 | 154 | 600 | 1:1 | 3.81 | 49.9 | (0.682, | 102 |
| | | | | | | 0.317) | |
| Example 39 | 240 | 601 | 1:1 | 3.70 | 56.9 | (0.682, | 112 |
| | | | | | | 0.317) | |
| Example 40 | 410 | 604 | 1:2 | 3.83 | 56.7 | (0.682, | 103 |
| | | | | | | 0.317) | |
| Example 41 | 252 | 612 | 1:1 | 3.75 | 60.3 | (0.681, | 106 |
| | | | | | | 0.319) | |
| Example 42 | 69 | 617 | 1:2 | 3.76 | 61.5 | (0.681, | 110 |
| | | | | | | 0.319) | |
| Example 43 | 330 | 620 | 1:3 | 3.76 | 64.7 | (0.682, | 150 |
| | | | | | | 0.317) | |
| Example 44 | 654 | 619 | 1:1 | 3.71 | 65.1 | (0.681, | 180 |
| | | | | | | 0.319) | |

As can be seen in Tables 6 and 7, it can be seen that when the compound of Chemical Formula 2 of the present invention has, as a substituent, an aryl amine, which has a better hole transport ability as a donor, that is, a higher donating strength, the balance effect with an acceptor is further enhanced to have a high HOMO level, and thus, the compound is suitable as a red host of an organic light emitting device, because the driving voltage is further reduced and the service life is increased.

In addition, when compared to the compound of the present invention, the compounds of Comparative Examples D and E do not allow electrons to be injected because the compounds do not have an acceptor with good electron transport ability, and as can be seen in Tables 6 and 7 above, it can be seen that the efficiency and service life of a device, in which Comparative Compounds D and E are used in an organic material layer, are reduced.

Furthermore, although the compounds of Comparative Examples G to I had reduced driving voltages as can be seen in Tables 6 and 7, it can be confirmed that the service life and efficiency are low even though aryl amine, which has a strong donor character, is substituted. This is because energy is not easily transferred to the red dopant due to the high T1 level according to the substituent position of the core of the heterocyclic compound, and the high resistance due to the large band gap leads to the deterioration in stability, thereby adversely affecting the service life.

Therefore, it can be confirmed that the compound of the present application may have a reduced band gap and a low T1 level, thereby remarkably improving the service life and efficiency.

Further, as can be seen in Table 7, it can be confirmed that when the heterocyclic compounds represented by Chemical Formulae 1 and 2 are simultaneously included in the organic material layer of the organic light emitting device, the driving voltage, efficiency and service life can be improved.

From this result, it can be expected that an exciplex phenomenon may occur when both compounds are included. The exciplex phenomenon is a phenomenon in which electron exchange between a molecule with strong donor properties and a molecule with strong acceptor properties releases energy at the donor (p-Host) HOMO level and acceptor (n-Host) LUMO level. When a donor with a good hole transport capacity (p-host) and an acceptor with a good electron transport capacity (n-host) are used as hosts for the light emitting layer, holes are injected into the p-host and electrons are injected into the n-host, so that the driving voltage can be lowered, which can help to improve the service life. When the exciplex phenomenon between two molecules occurs as described above, a reverse intersystem crossing (RISC) occurs, and the internal quantum efficiency can be increased to 100% due to the RISC.

In particular, the heterocyclic compound of Chemical Formula 2 is a bipolar compound and does not have a strong acceptor ability, but as an acceptor (n-host), which is the heterocyclic compound represented by Chemical Formula 1 with good electron transport ability, is injected, red-shifted photoluminescence (PL) changes may be shown to form an exciplex, thereby helping to improve the light emitting characteristics. In addition, it could be confirmed that as the acceptor (n-host), which is the heterocyclic compound of Chemical Formula 1 with good electron transport ability, was injected, the service life could be remarkably improved due to the appropriate movement of the light emitting zone in the light emitting layer.

### Experimental Example 2

(1) Manufacture of Organic Light Emitting Device (2Stack Red N+P Mixed Host)

A glass substrate, in which indium tin oxide (ITO) was thinly coated to have a thickness of 1,500 Å, was ultrasonically washed with distilled water. When the washing with distilled water was finished, the glass substrate was ultrasonically washed with a solvent such as acetone, methanol, and isopropyl alcohol, dried and then subjected to ultraviolet ozone (UVO) treatment for 5 minutes using UV in an ultraviolet (UV) washing machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition. As the common layers, the hole injection layer 4,4′,4″-tris[2-naphthyl(phenyl)amino]triphenylamine (2-TNATA) and the hole transport layer N,N′-di(1-naphthyl)-N,N′-diphenyl-(1,1″-biphenyl)-4,4′-diamine (NPB) were formed on the ITO transparent electrode (positive electrode).

A light emitting layer was thermally vacuum deposited thereon as follows. The light emitting layer was deposited to have a thickness of 400 Å by using the compounds represented by Chemical Formulae 1 and 2 as red hosts and (piq)₂(Ir) (acac) as a red phosphorescent dopant to dope the host with (piq)₂(Ir) (acac) in an amount of 2%.

Thereafter, Alq₃ was deposited to have a thickness of 120 Å as an electron transport layer, Bphen was deposited to have a thickness of 120 Å as a charge generation layer thereon, and MoO₃ was also deposited to have a thickness of 100 Å as a charge generation layer thereon. A hole transport layer N,N′-Di(1-naphthyl)-N,N′-diphenyl-(1,1′-biphenyl)-4,4′-diamine (NPB) was formed. A light emitting layer was thermally vacuum deposited thereon as follows. The light emitting layer was deposited to have a thickness of 400 Å by using the compounds represented by Chemical Formulae 1 and 2 as red hosts and (piq)₂(Ir) (acac) as a red phosphorescent dopant to dope the host with (piq)₂(Ir) (acac) in an amount of 2%.

Thereafter, Alq₃ was deposited to have a thickness of 300 Å as an electron transport layer. Finally, lithium fluoride (LiF) was deposited to have a thickness of 20 Å on the electron transport layer to form an electron injection layer, and then aluminum (Al) negative electrode was deposited to have a thickness of 1,200 Å on the electron injection layer to form a negative electrode, thereby manufacturing an organic electroluminescence device. Meanwhile, all the organic compounds required for manufacturing an OLED were subjected to vacuum sublimed purification under 10⁻⁶ to 10⁻⁸ torr for each material, and then used for the manufacture of OLED.

For the organic electroluminescence device manufactured as described above, electroluminescence (EL) characteristics were measured by M7000 manufactured by McScience Inc., and based on the measurement result thereof, T90 was measured by a service life measurement equipment (M6000) manufactured by McScience Inc., when the reference luminance was 6,000 cd/m². The characteristics of the organic electroluminescence device of the present invention are as shown in the following Table 8.

TABLE 8

| | | | | | | Color | |
|---|---|---|---|---|---|---|---|
| | | | | Driving | Effi- | coor- | Service |
| | First | Second | Ratio | voltage | ciency | dinate | life |
| | host | host | (N:P) | (V) | (cd/A) | (x, y) | (T95) |
| Comparative | A | 401 | 1:1 | 6.75 | 64.4 | (0.682, | 140 |
| Example 43 | | | | | | 0.317) | |
| Example 45 | 240 | | | 5.76 | 119.8 | (0.682, | 263 |
| | | | | | | 0.317) | |
| Comparative | 252 | E | | 7.87 | 84 | (0.681, | 152 |
| Example 44 | | | | | | 0.319) | |
| Example 46 | | 494 | | 5.68 | 124.6 | (0.681, | 269 |
| | | | | | | 0.319) | |
| Comparative | 96 | I | 1:2 | 6.64 | 88 | (0.681, | 189 |
| Example 45 | | | | | | 0.319) | |
| Example 47 | | 346 | | 5.92 | 116.6 | (0.682, | 250 |
| | | | | | | 0.317) | |
| Example 48 | 216 | 581 | 1:3 | 5.66 | 130.6 | (0.682, | 320 |
| | | | | | | 0.317) | |
| Comparative | | D | | 6.78 | 57 | (0.682, | 154 |
| Example 46 | | | | | | 0.317) | |
| Example 49 | 226 | 571 | | 5.84 | 111.4 | (0.682, | 248 |
| | | | | | | 0.317) | |
| Example 50 | 176 | 378 | | 5.72 | 104.6 | (0.681, | 254 |
| | | | | | | 0.319) | |
| Example 51 | 326 | 581 | 1:2 | 5.66 | 137.6 | (0.681, | 332 |
| | | | | | | 0.319) | |
| Comparative | 28 | C | | 6.04 | 100 | (0.681, | 156 |
| Example 47 | | | | | | 0.319) | |
| Comparative | 318 | F | | 6.78 | 77.2 | (0.681, | 137 |
| Example 48 | | | | | | 0.319) | |
| Example 52 | 51 | 338 | 1:1 | 5.85 | 122.4 | (0.681, | 259 |
| | | | | | | 0.319) | |
| Example 53 | 280 | | | 6.02 | 113.6 | (0.682, | 244 |
| | | | | | | 0.317) | |
| Comparative | B | 353 | | 6.38 | 57.8 | (0.682, | 140 |
| Example 49 | | | | | | 0.317) | |
| Example 54 | 154 | | | 6.06 | 99.8 | (0.682, | 238 |
| | | | | | | 0.317) | |
| Example 55 | 12 | 460 | 2:1 | 5.60 | 117.4 | (0.681, | 242 |
| | | | | | | 0.319) | |
| Example 56 | 75 | 545 | 2:1 | 6.25 | 116 | (0.681, | 257 |
| | | | | | | 0.319) | |
| Example 57 | 330 | 582 | 1:3 | 5.79 | 132.2 | (0.682, | 312 |
| | | | | | | 0.317) | |
| Example 58 | 323 | 338 | 1:1 | 6.01 | 111.8 | (0.682, | 234 |
| | | | | | | 0.317) | |
| Comparative | 651 | C | 1:2 | 6.23 | 102 | (0.681, | 158 |
| Example 50 | | | | | | 0.319) | |
| Example 59 | 4 | 460 | 1:1 | 5.72 | 108.8 | (0.681, | 236 |
| | | | | | | 0.319) | |
| Example 60 | 22 | 571 | 2:1 | 6.16 | 112 | (0.682, | 248 |
| | | | | | | 0.317) | |
| Example 61 | 652 | 332 | 2:1 | 5.78 | 120.2 | (0.682, | 294 |
| | | | | | | 0.317) | |
| Example 62 | 654 | 581 | 1:1 | 5.62 | 137.6 | (0.681, | 339 |
| | | | | | | 0.319) | |
| Example 63 | 660 | 580 | 1:1 | 5.69 | 120.6 | (0.682, | 308 |
| | | | | | | 0.317) | |
| Example 64 | 298 | 410 | 1:2 | 5.93 | 117.4 | (0.682, | 265 |
| | | | | | | 0.317) | |
| Example 65 | 51 | 422 | 1:1 | 5.97 | 118.4 | (0.681, | 195 |
| | | | | | | 0.319) | |
| Example 66 | 318 | 549 | 1:1 | 6.12 | 109.6 | (0.682, | 191 |
| | | | | | | 0.317) | |
| Example 67 | 154 | 340 | 1:1 | 5.883 | 101.8 | (0.682, | 205 |
| | | | | | | 0.317) | |
| Example 68 | 330 | 589 | 1:3 | 5.72 | 136.2 | (0.682, | 240 |
| | | | | | | 0.317) | |
| Example 69 | 84 | 594 | 1:2 | 5.77 | 121.4 | (0.682, | 265 |
| | | | | | | 0.317) | |
| Example 70 | 55 | 584 | 1:1 | 5.79 | 124.4 | (0.681, | 209 |
| | | | | | | 0.319) | |
| Example 71 | 69 | 520 | 1:2 | 5.80 | 125.6 | (0.681, | 226 |
| | | | | | | 0.319) | |

As shown in Table 8, it could be confirmed that when an organic light emitting device was manufactured by stacking two stacks of light emitting layers including both the compound represented by Chemical Formula 1 and the compound represented by Chemical Formula 2 of the present invention, the light emitting layer was deposited twice, and thus, the efficiency was increased compared to the case of manufacturing an organic light emitting device with a single stack.

Meanwhile, it could be confirmed that even in the organic light emitting device including two stacks, as in the case of the organic light emitting device manufactured with a single stack as in Experimental Example 1, driving voltage could be remarkably improved because the hole mobility is increased by simultaneously including the compounds of Chemical Formulae 1 and 2 in the organic material layer of the organic light emitting device. Furthermore, it could be confirmed that the efficiency is also improved by decrease in current leakage through electron block and electron confinement.

What is claimed is:

1. An organic light emitting device comprising: a first electrode; a second electrode; and an organic material layer provided between the first electrode and the second electrode,
wherein the organic material layer comprises a compound of the following Chemical Formula 1 and a compound of the following Chemical Formula 2: 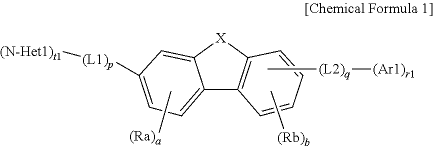
X is O or S,
Ar1 is a substituted or unsubstituted C6 to C60 aryl group; or a substituted or unsubstituted C2 to C60 heteroaryl group,
N-Het1 is a C2 to C60 heteroaryl group which is substituted or unsubstituted and includes N,
t1 and r1 are each an integer from 1 to 3, and when t1 is 2 or higher, N-Het1's are the same as or different from each other, and when r1 is 2 or higher, Ar1's are the same as or different from each other,
L1 and L2 are the same as or different from each other, and are each independently a direct bond; a substituted or unsubstituted C6 to C60 arylene group; or a substituted or unsubstituted C2 to C60 heteroarylene group,
p and q are each an integer from 0 to 3, and when p is 2 or higher, L1's are the same as or different from each other, and when q is 2 or higher, L2's are the same as or different from each other,
Ra and Rb are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and
a and b are each an integer from 0 to 3, and when a is 2 or higher, Ra's are the same as or different from each other, and when b is 2 or higher, Rb's are the same as or different from each other, 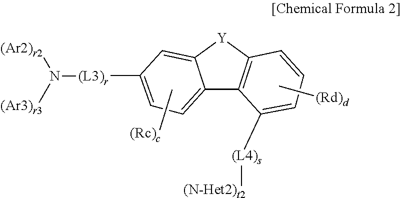
Y is O or S,
Ar2 and Ar3 are the same as or different from each other, and are each independently a substituted or unsubstituted C6 to C60 aryl group; or a substituted or unsubstituted C2 to C60 heteroaryl group,
N-Het2 is a C2 to C60 heteroaryl group which is substituted or unsubstituted and includes N,
t2, r2 and r3 are each an integer from 1 to 3, and when t2 is 2 or higher, N-Het2's are the same as or different from each other, and when r2 is 2 or higher, Ar2's are the same as or different from each other, and when r3 is 2 or higher, Ar3's are the same as or different from each other,
L3 and L4 are the same as or different from each other, and are each independently a direct bond; a substituted or unsubstituted C6 to C60 arylene group; or a substituted or unsubstituted C2 to C60 heteroarylene group,
r and s are each an integer from 0 to 3, and when r is 2 or higher, L3's are the same as or different from each other, and when s is 2 or higher, L4's are the same as or different from each other,
Rc and Rd are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and
c and d are each an integer from 0 to 3, and when c is 2 or higher, Rc's are the same as or different from each other, and when d is 2 or higher, Rd's are the same as or different from each other.

2. The organic light emitting device of claim 1, wherein Chemical Formula 1 is represented by any one of the following Chemical Formulae 1-1 to 1-3: 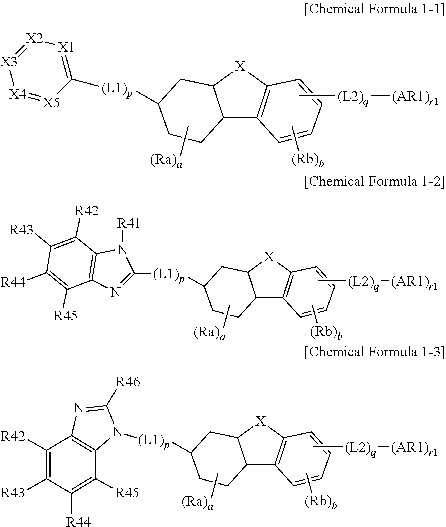
in Chemical Formulae 1-1 to 1-3,
the definitions of X, L1, L2, Ar1, Ra, Rb, r1, p, q, a and b are the same as the definitions in Chemical Formula 1,
X1 is CR11 or N, X2 is CR12 or N, X3 is CR13 or N, X4 is CR14 or N, X5 is CR15 or N, and at least one of X1 to X5 is N, and
R11 to R15 and R41 to R46 are the same as or different from each other, and are each independently hydrogen; deuterium; a halogen; a cyano group; a substituted or unsubstituted C1 to C60 alkyl group; a substituted or unsubstituted C3 to C60 cycloalkyl group; a substituted or unsubstituted C3 to C60 heterocycloalkyl group; a substituted or unsubstituted C6 to C60 aryl group; or a substituted or unsubstituted C2 to C60 heteroaryl group.

3. The organic light emitting device of claim 1, wherein Chemical Formula 1 is represented by any one of the following Chemical Formulae 1-4 to 1-6: 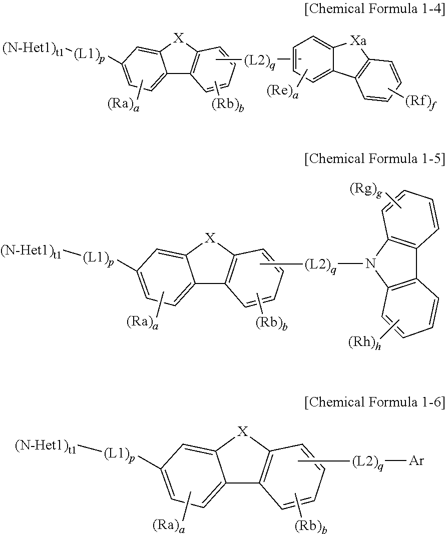
in Chemical Formulae 1-4 to 1-6,
the definitions of X, L1, L2, N-Het1, Ra, Rb, t1, p, q, a and b are the same as the definitions in Chemical Formula 1,
Xa is S, O or N(R51),
Re, Rf, Rg, Rh and R51 are the same as or different from each other, and are each independently selected from the group consisting of hydrogen; deuterium; a halogen; a cyano group; a substituted or unsubstituted C1 to C60 alkyl group; a substituted or unsubstituted C3 to C60 cycloalkyl group; a substituted or unsubstituted C3 to C60 heterocycloalkyl group; a substituted or unsubstituted C6 to C60 aryl group; a substituted or unsubstituted C2 to C60 heteroaryl group; a substituted or unsubstituted phosphine oxide group; and a substituted or unsubstituted amine group, or two or more adjacent groups are bonded to each other to form a substituted or unsubstituted aliphatic or aromatic hydrocarbon ring or hetero ring,
e is an integer from 0 to 3, f, g and h are each an integer from 0 to 4, and when e is 2 or higher, Re's are the same as or different from each other, and when f is 2 or higher, Rf's are the same as or different from each other, and when g is 2 or higher, Rg's are the same as or different from each other, and when h is 2 or higher, Rh's are the same as or different from each other, and
Ar is a substituted or unsubstituted C6 to C60 aryl group.

4. The organic light emitting device of claim 1, wherein Chemical Formula 1 is represented by any one of the following Chemical Formulae 1-7 and 1-8: 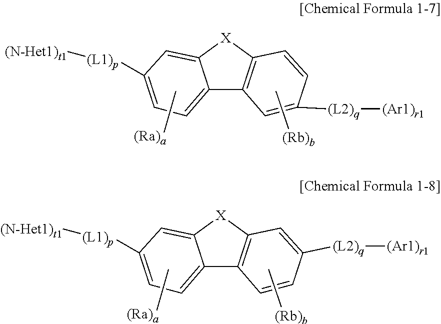
in Chemical Formulae 1-7 and 1-8,
the definitions of X, L1, L2, Ar1, N-Het1, Ra, Rb, t1, r1, p, q, a and b are the same as the definitions in Chemical Formula 1.

5. The organic light emitting device of claim 1, wherein a deuterium content of the compound of Chemical Formula 1 is 0% or 1% to 100%.

6. The organic light emitting device of claim 1, wherein Chemical Formula 1 is represented by any one of the following compounds:

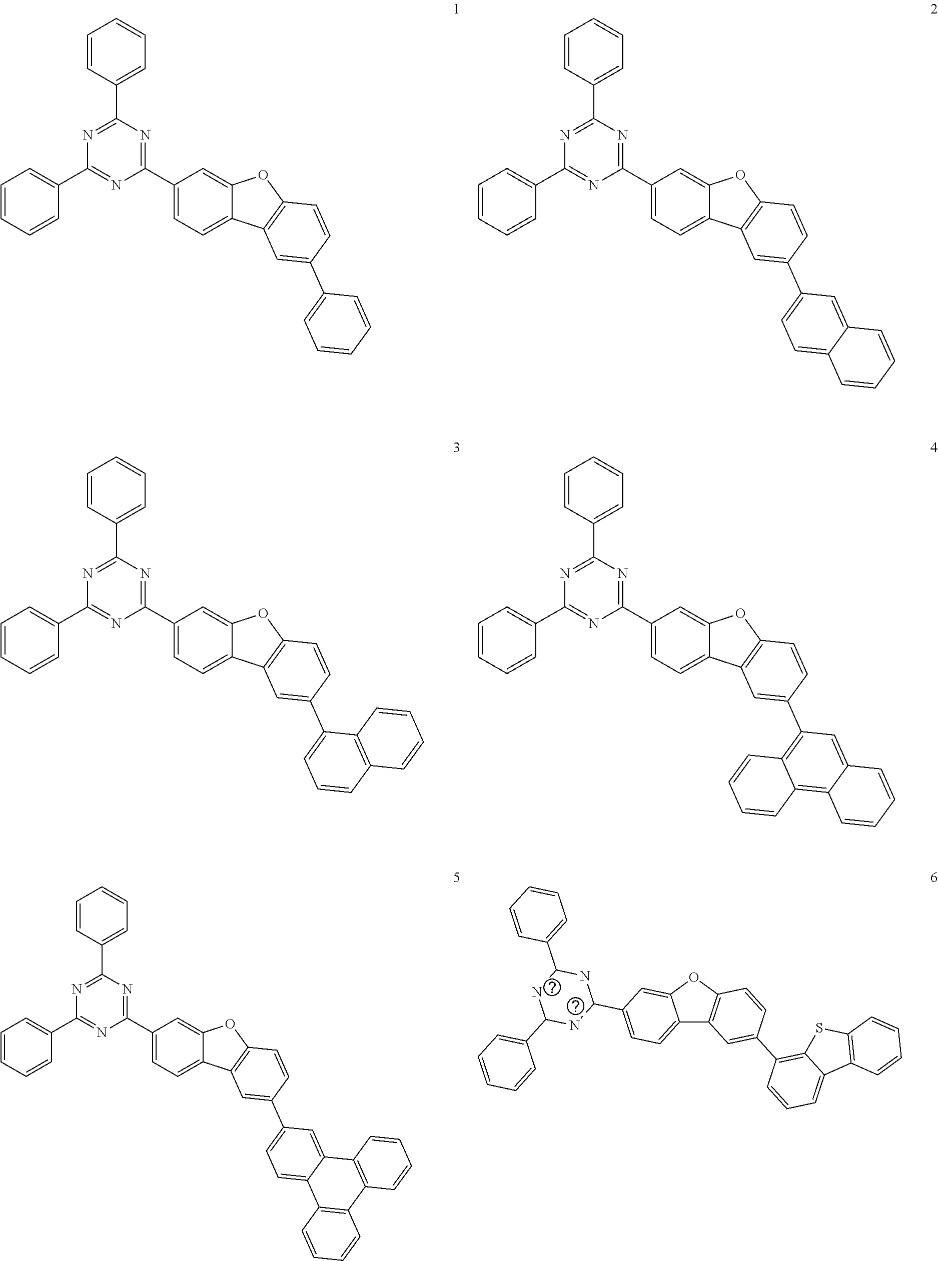 [SMILES: c1ccc(-c2ccc3oc4cc(-c5nc(-c6ccccc6)nc(-c6ccccc6)n5)ccc4c3c2)cc1 ; c1ccc(-c2nc(-c3ccccc3)nc(-c3ccc4c(c3)oc3ccc(-c5ccc6ccccc6c5)cc34)n2)cc1 ; c1ccc(-c2nc(-c3ccccc3)nc(-c3ccc4c(c3)oc3ccc(-c5cccc6ccccc56)cc34)n2)cc1 ; c1ccc(-c2nc(-c3ccccc3)nc(-c3ccc4c(c3)oc3ccc(-c5cc6ccccc6c6ccccc56)cc34)n2)cc1 ; c1ccc(-c2nc(-c3ccccc3)nc(-c3ccc4c(c3)oc3ccc(-c5ccc6c7ccccc7c7ccccc7c6c5)cc34)n2)cc1 ; c1ccc(C2NC(c3ccccc3)NC(c3ccc4c(c3)oc3ccc(-c5cccc6c5sc5ccccc56)cc34)N2)cc1]

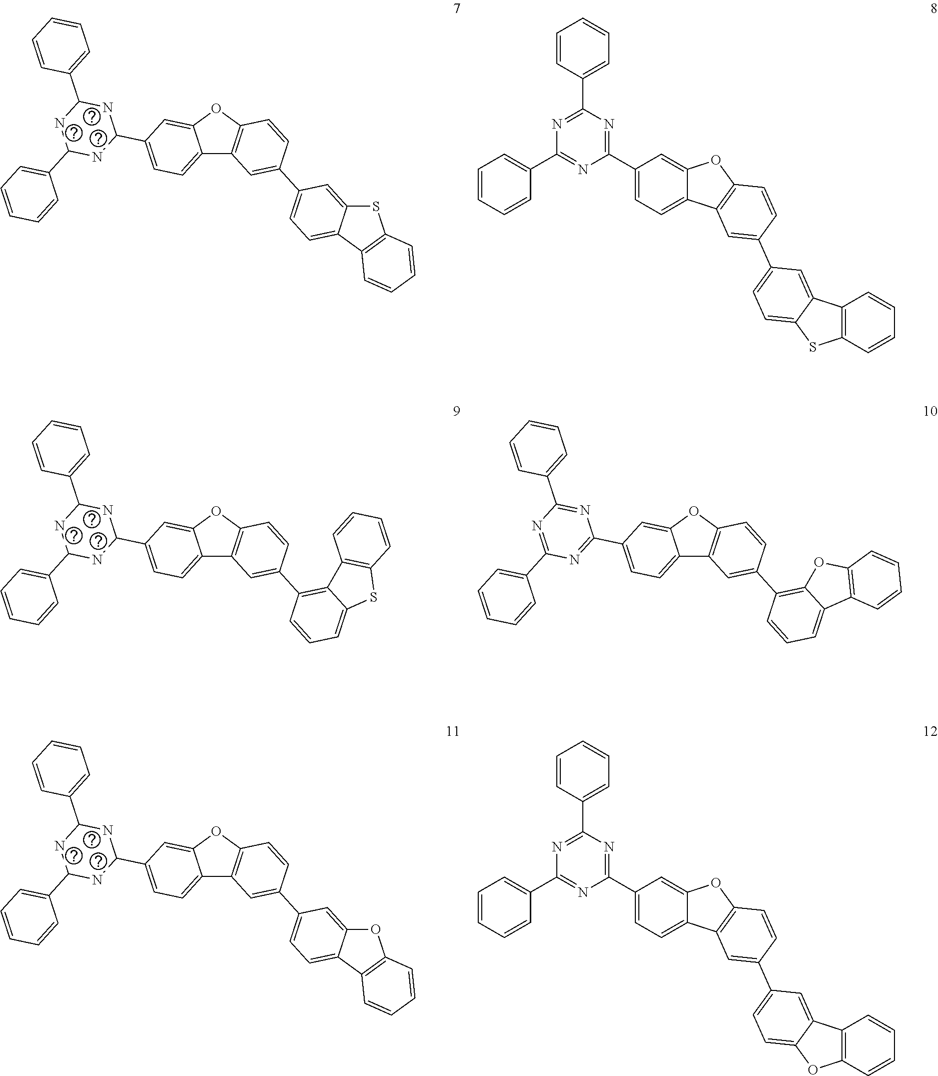 [SMILES: c1ccc(C2NC(c3ccccc3)NC(c3ccc4c(c3)oc3ccc(-c5ccc6c(c5)sc5ccccc56)cc34)N2)cc1 ; c1ccc(-c2nc(-c3ccccc3)nc(-c3ccc4c(c3)oc3ccc(-c5ccc6sc7ccccc7c6c5)cc34)n2)cc1 ; c1ccc(C2NC(c3ccccc3)NC(c3ccc4c(c3)oc3ccc(-c5cccc6sc7ccccc7c56)cc34)N2)cc1 ; c1ccc(-c2nc(-c3ccccc3)nc(-c3ccc4c(c3)oc3ccc(-c5cccc6c5oc5ccccc56)cc34)n2)cc1 ; c1ccc(C2NC(c3ccccc3)NC(c3ccc4c(c3)oc3ccc(-c5ccc6c(c5)oc5ccccc56)cc34)N2)cc1 ; c1ccc(-c2nc(-c3ccccc3)nc(-c3ccc4c(c3)oc3ccc(-c5ccc6oc7ccccc7c6c5)cc34)n2)cc1]

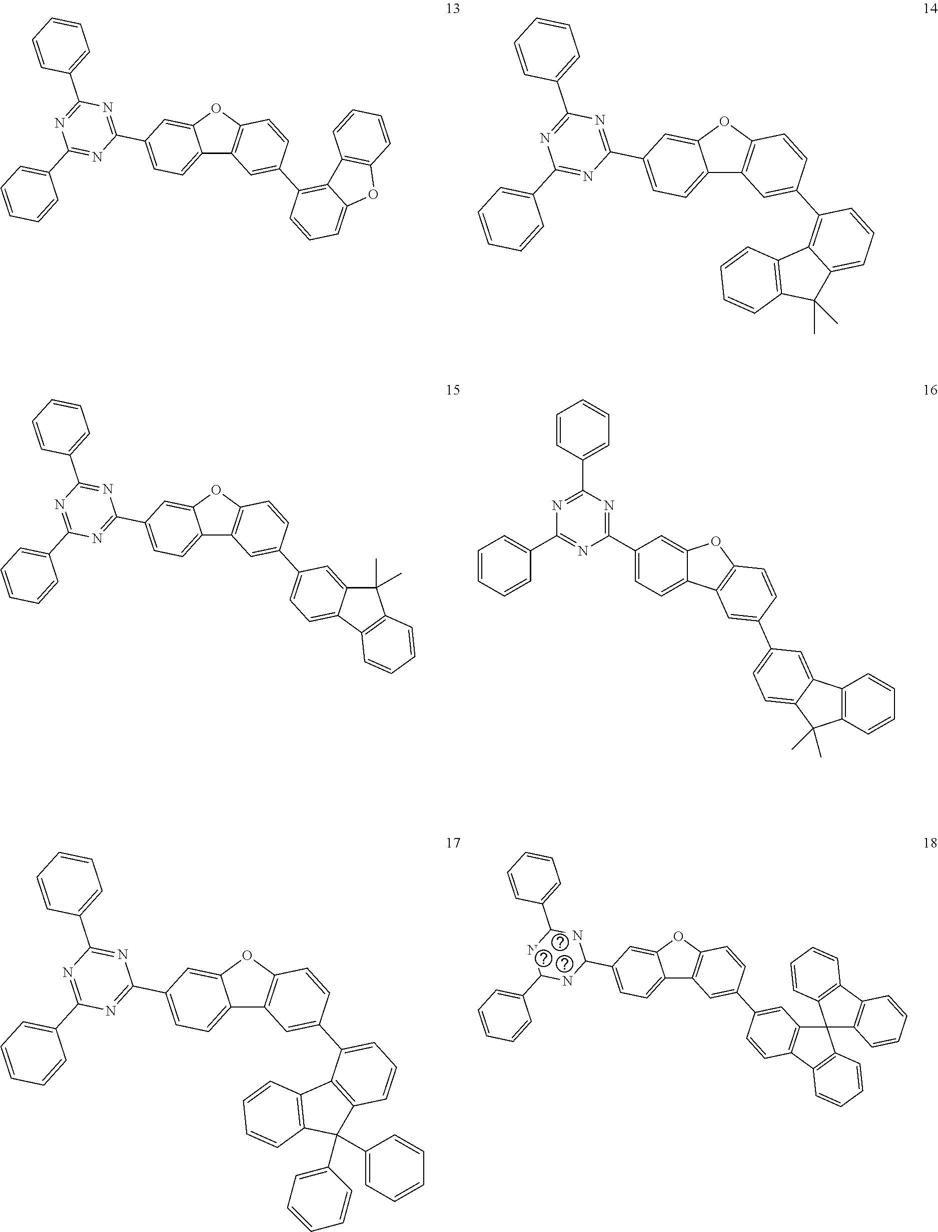

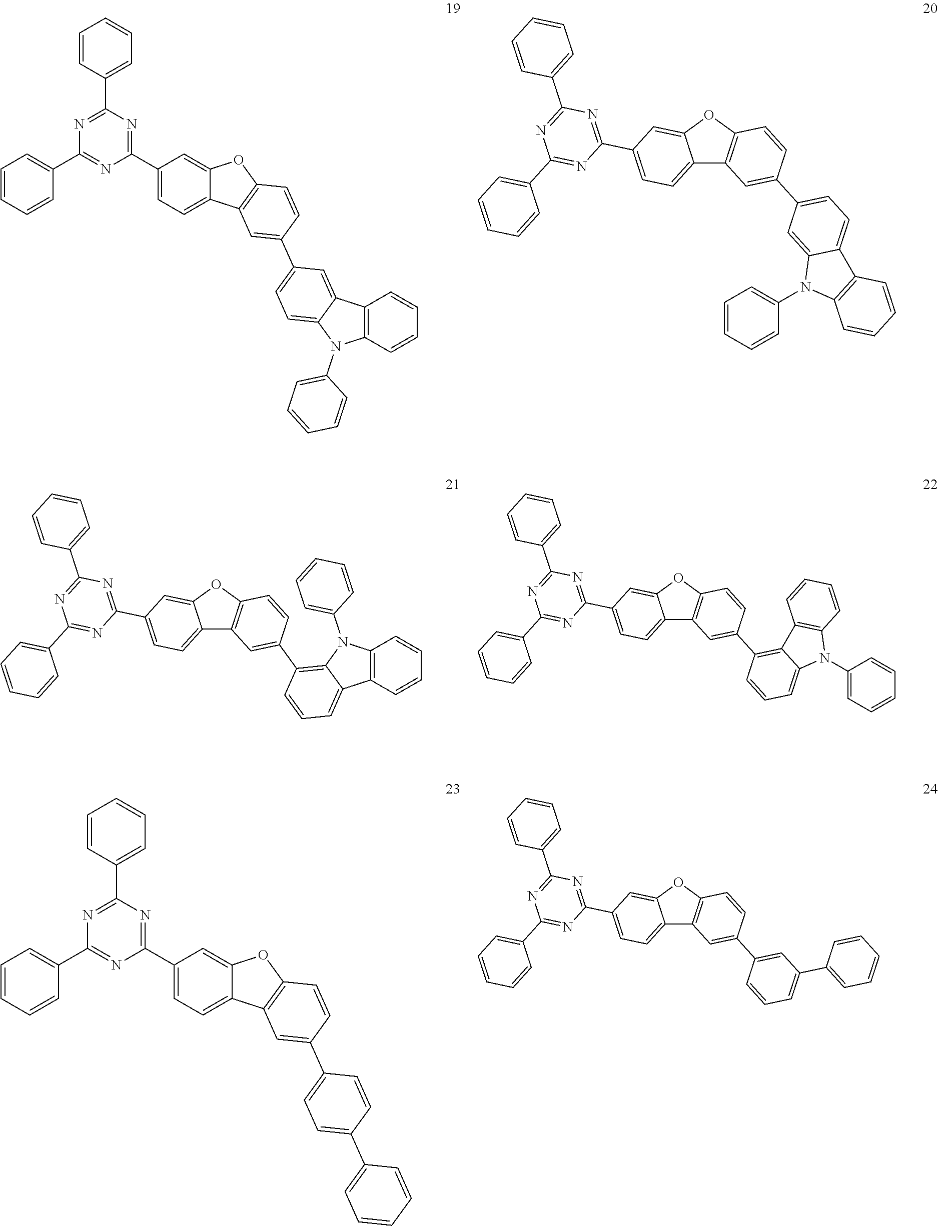

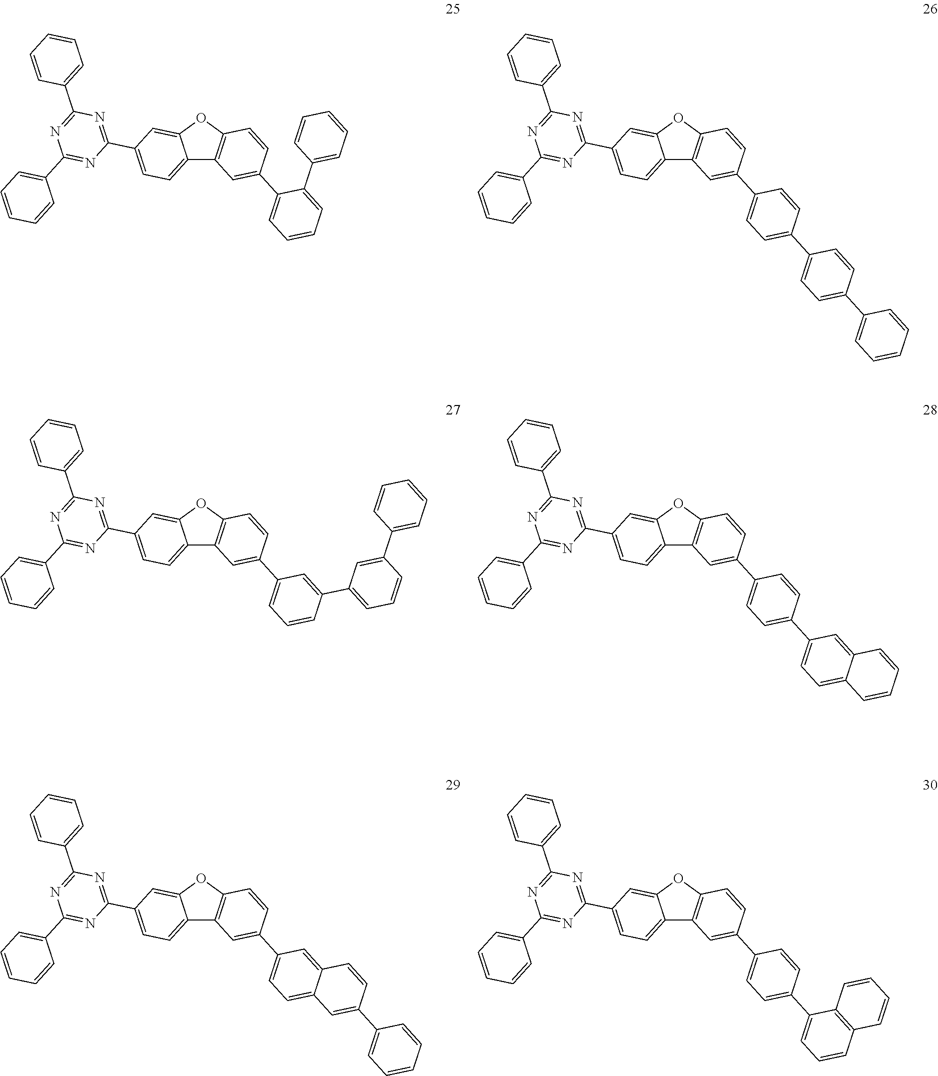

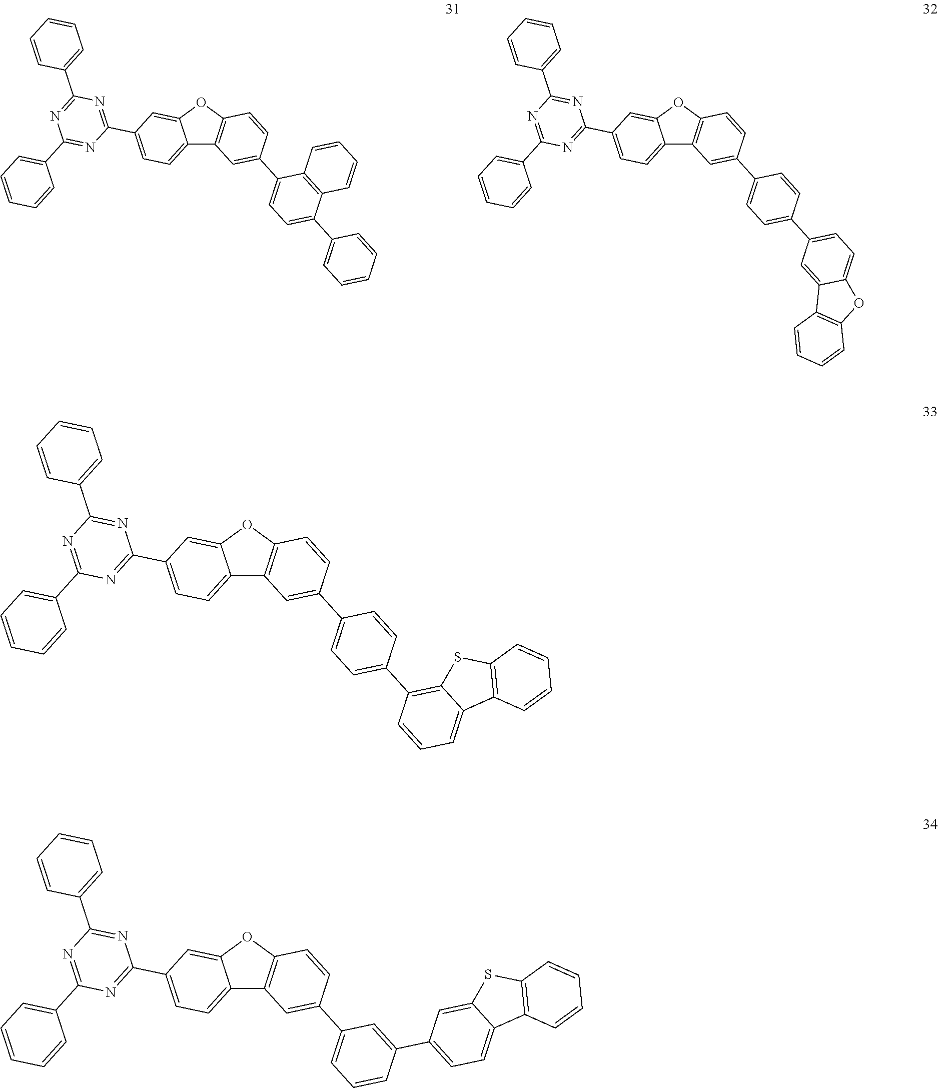 [SMILES: c1ccc(-c2nc(-c3ccccc3)nc(-c3ccc4c(c3)oc3ccc(-c5ccc(-c6ccccc6)c6ccccc56)cc34)n2)cc1 ; c1ccc(-c2nc(-c3ccccc3)nc(-c3ccc4c(c3)oc3ccc(-c5ccc(-c6ccc7oc8ccccc8c7c6)cc5)cc34)n2)cc1 ; c1ccc(-c2nc(-c3ccccc3)nc(-c3ccc4c(c3)oc3ccc(-c5ccc(-c6cccc7c6sc6ccccc67)cc5)cc34)n2)cc1 ; c1ccc(-c2nc(-c3ccccc3)nc(-c3ccc4c(c3)oc3ccc(-c5cccc(-c6ccc7c(c6)sc6ccccc67)c5)cc34)n2)cc1]

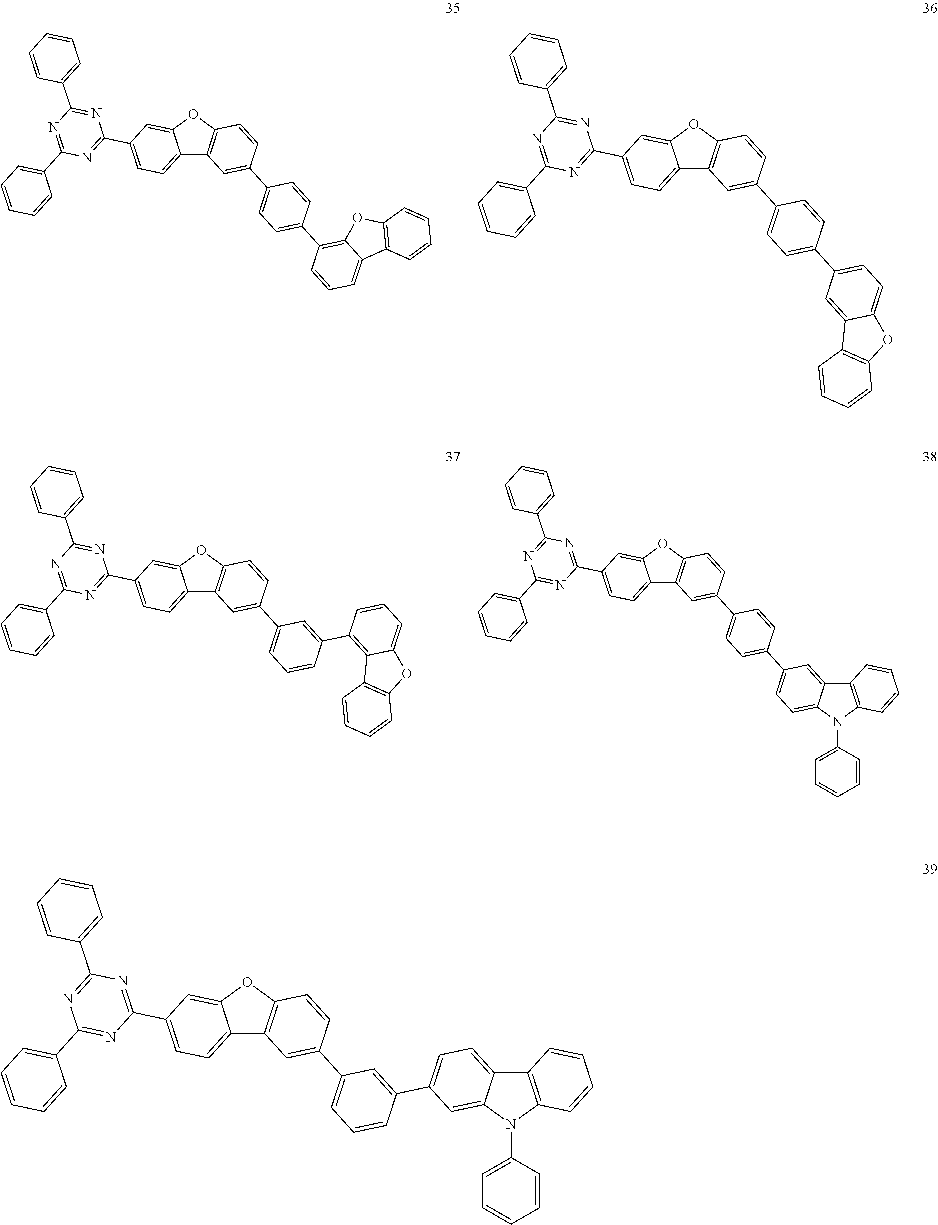 [SMILES: c1ccc(-c2nc(-c3ccccc3)nc(-c3ccc4c(c3)oc3ccc(-c5ccc(-c6cccc7c6oc6ccccc67)cc5)cc34)n2)cc1 ; c1ccc(-c2nc(-c3ccccc3)nc(-c3ccc4c(c3)oc3ccc(-c5ccc(-c6ccc7oc8ccccc8c7c6)cc5)cc34)n2)cc1 ; c1ccc(-c2nc(-c3ccccc3)nc(-c3ccc4c(c3)oc3ccc(-c5cccc(-c6cccc7oc8ccccc8c67)c5)cc34)n2)cc1 ; c1ccc(-c2nc(-c3ccccc3)nc(-c3ccc4c(c3)oc3ccc(-c5ccc(-c6ccc7c(c6)c6ccccc6n7-c6ccccc6)cc5)cc34)n2)cc1 ; c1ccc(-c2nc(-c3ccccc3)nc(-c3ccc4c(c3)oc3ccc(-c5cccc(-c6ccc7c8ccccc8n(-c8ccccc8)c7c6)c5)cc34)n2)cc1]

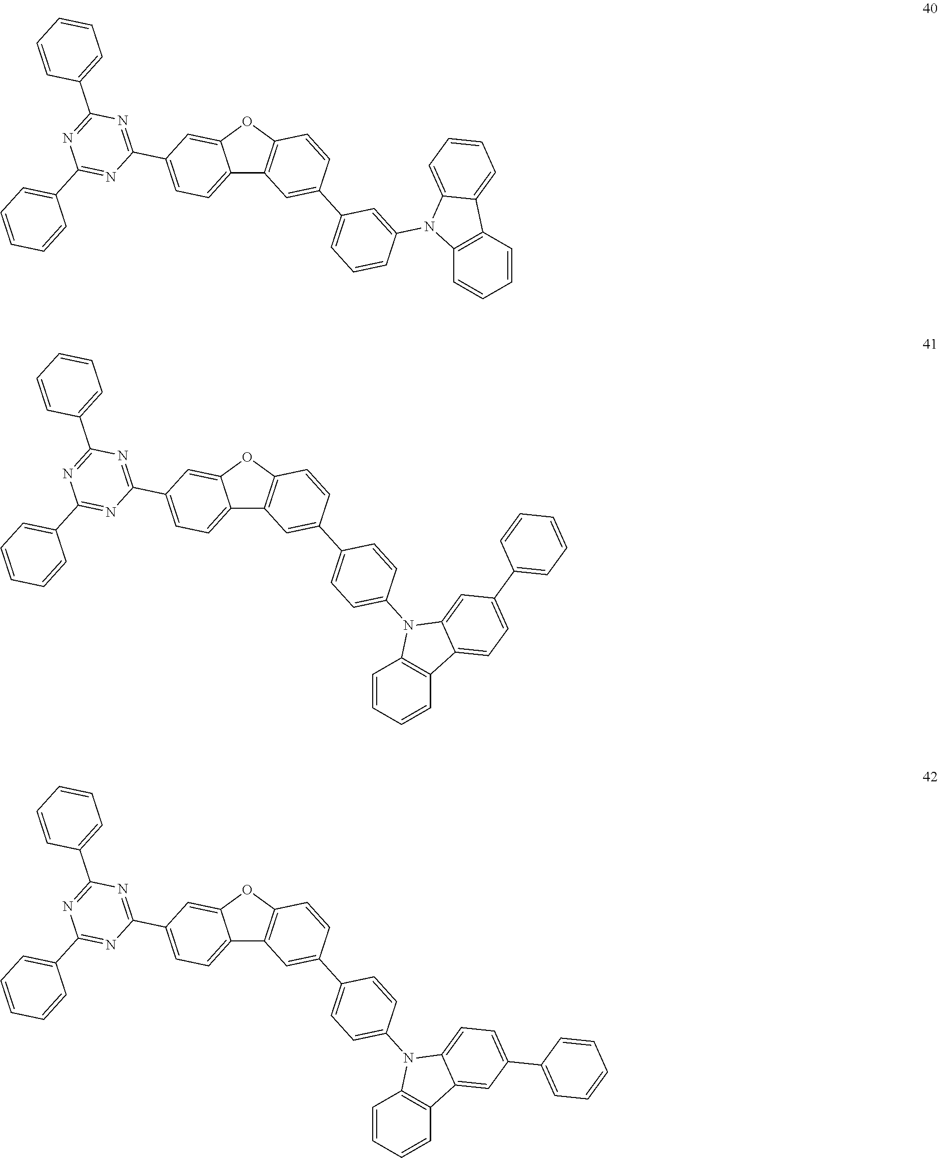 [SMILES: c1ccc(-c2nc(-c3ccccc3)nc(-c3ccc4c(c3)oc3ccc(-c5cccc(-n6c7ccccc7c7ccccc76)c5)cc34)n2)cc1 ; c1ccc(-c2ccc3c4ccccc4n(-c4ccc(-c5ccc6oc7cc(-c8nc(-c9ccccc9)nc(-c9ccccc9)n8)ccc7c6c5)cc4)c3c2)cc1 ; c1ccc(-c2ccc3c(c2)c2ccccc2n3-c2ccc(-c3ccc4oc5cc(-c6nc(-c7ccccc7)nc(-c7ccccc7)n6)ccc5c4c3)cc2)cc1]

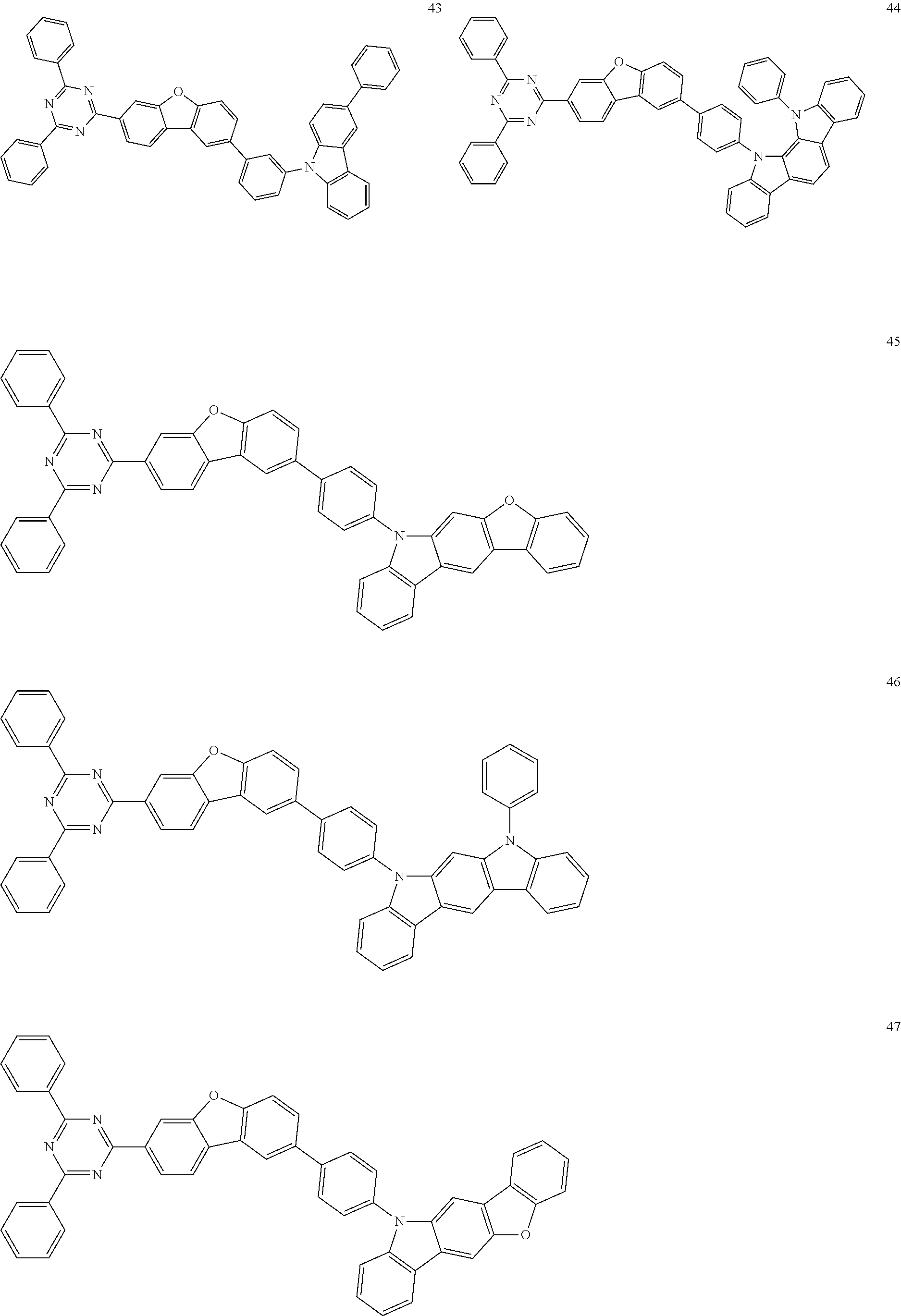

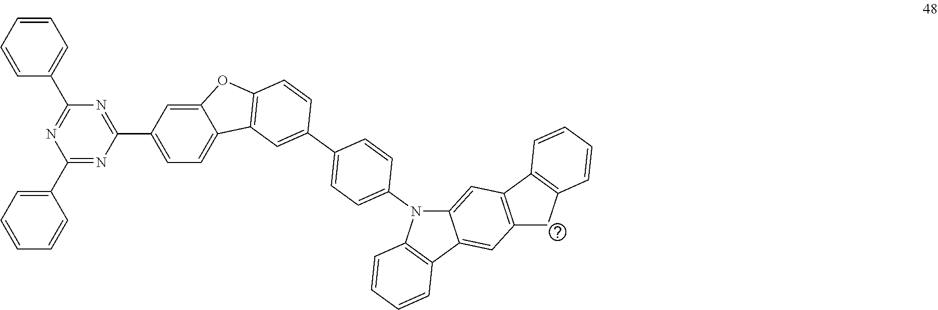 [SMILES: c1ccc(-c2nc(-c3ccccc3)nc(-c3ccc4c(c3)oc3ccc(-c5ccc(-n6c7ccccc7c7cc8c(cc76)-c6ccccc6C8)cc5)cc34)n2)cc1]

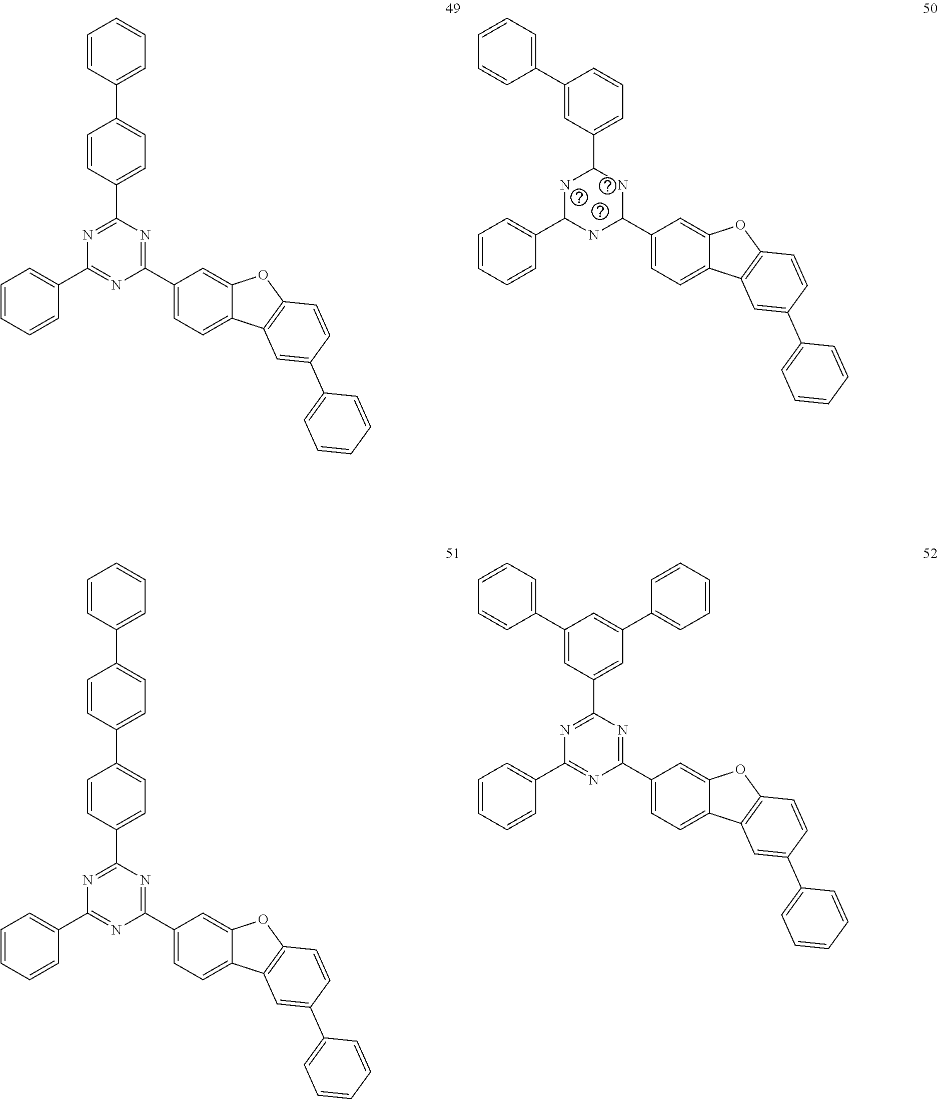 [SMILES: c1ccc(-c2ccc(-c3nc(-c4ccccc4)nc(-c4ccc5c(c4)oc4ccc(-c6ccccc6)cc45)n3)cc2)cc1 ; c1ccc(-c2cccc(C3NC(c4ccccc4)NC(c4ccc5c(c4)oc4ccc(-c6ccccc6)cc45)N3)c2)cc1 ; c1ccc(-c2ccc(-c3ccc(-c4nc(-c5ccccc5)nc(-c5ccc6c(c5)oc5ccc(-c7ccccc7)cc56)n4)cc3)cc2)cc1 ; c1ccc(-c2cc(-c3ccccc3)cc(-c3nc(-c4ccccc4)nc(-c4ccc5c(c4)oc4ccc(-c6ccccc6)cc45)n3)c2)cc1]

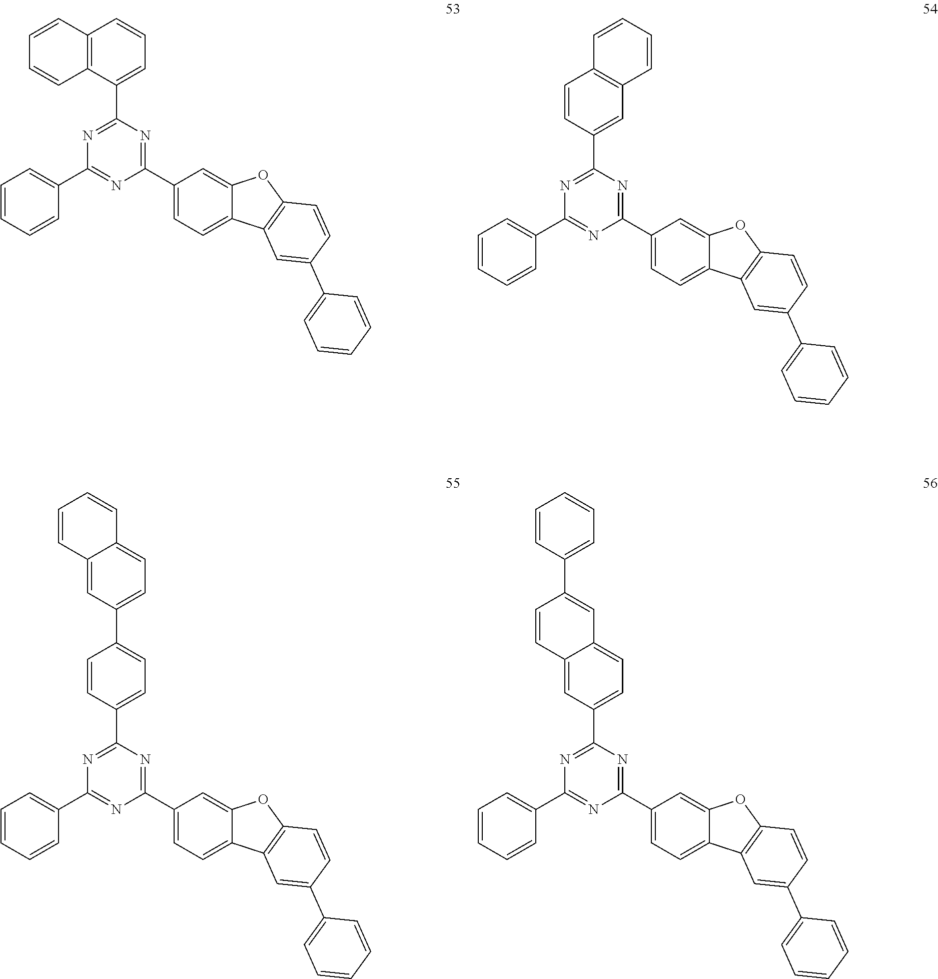 [SMILES: c1ccc(-c2ccc3oc4cc(-c5nc(-c6ccccc6)nc(-c6cccc7ccccc67)n5)ccc4c3c2)cc1 ; c1ccc(-c2ccc3oc4cc(-c5nc(-c6ccccc6)nc(-c6ccc7ccccc7c6)n5)ccc4c3c2)cc1 ; c1ccc(-c2ccc3oc4cc(-c5nc(-c6ccccc6)nc(-c6ccc(-c7ccc8ccccc8c7)cc6)n5)ccc4c3c2)cc1 ; c1ccc(-c2ccc3cc(-c4nc(-c5ccccc5)nc(-c5ccc6c(c5)oc5ccc(-c7ccccc7)cc56)n4)ccc3c2)cc1]

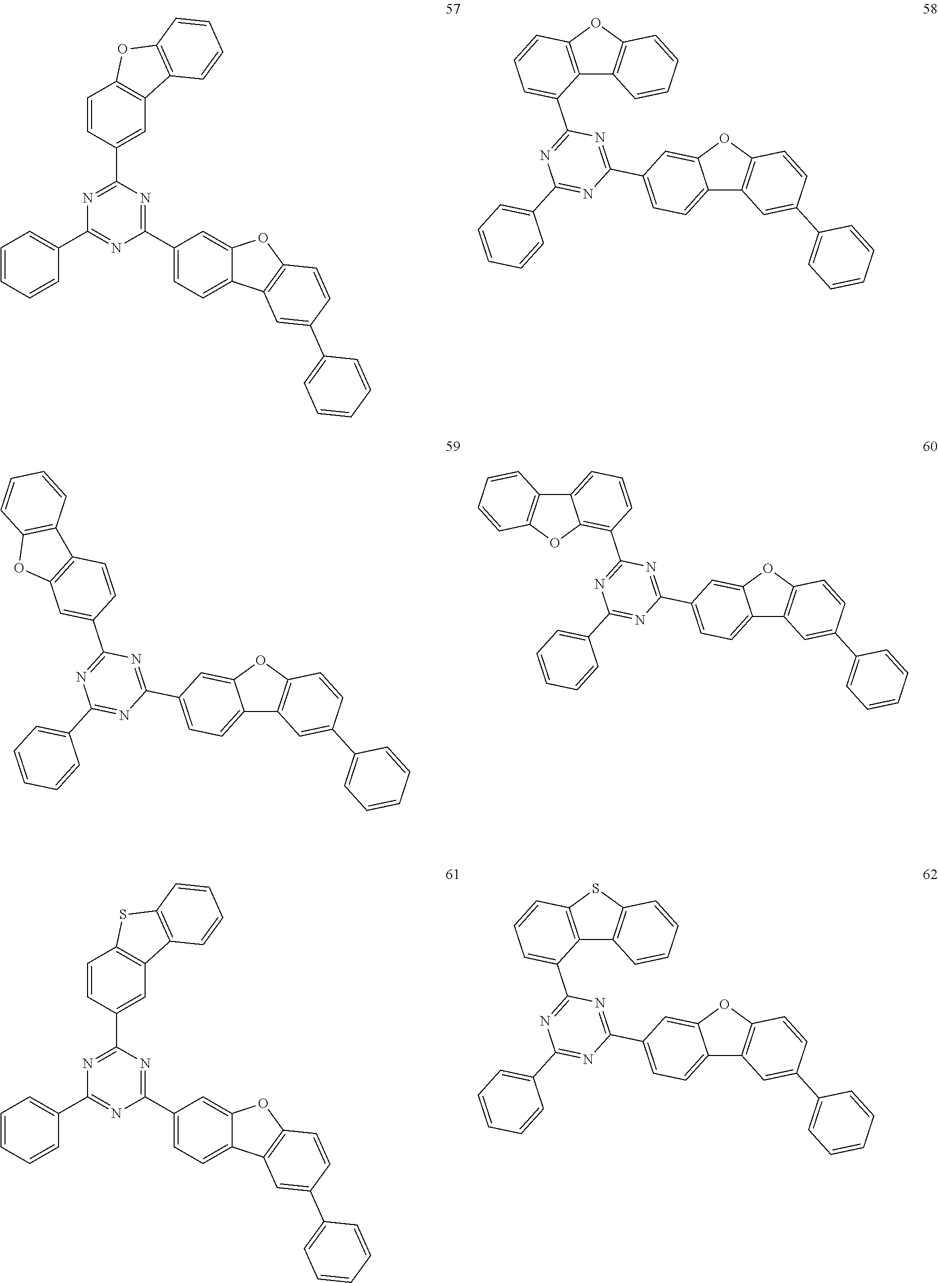 [SMILES: c1ccc(-c2ccc3oc4cc(-c5nc(-c6ccccc6)nc(-c6ccc7oc8ccccc8c7c6)n5)ccc4c3c2)cc1 ; c1ccc(-c2ccc3oc4cc(-c5nc(-c6ccccc6)nc(-c6cccc7oc8ccccc8c67)n5)ccc4c3c2)cc1 ; c1ccc(-c2ccc3oc4cc(-c5nc(-c6ccccc6)nc(-c6ccc7c(c6)oc6ccccc67)n5)ccc4c3c2)cc1 ; c1ccc(-c2ccc3oc4cc(-c5nc(-c6ccccc6)nc(-c6cccc7c6oc6ccccc67)n5)ccc4c3c2)cc1 ; c1ccc(-c2ccc3oc4cc(-c5nc(-c6ccccc6)nc(-c6ccc7sc8ccccc8c7c6)n5)ccc4c3c2)cc1 ; c1ccc(-c2ccc3oc4cc(-c5nc(-c6ccccc6)nc(-c6cccc7sc8ccccc8c67)n5)ccc4c3c2)cc1]

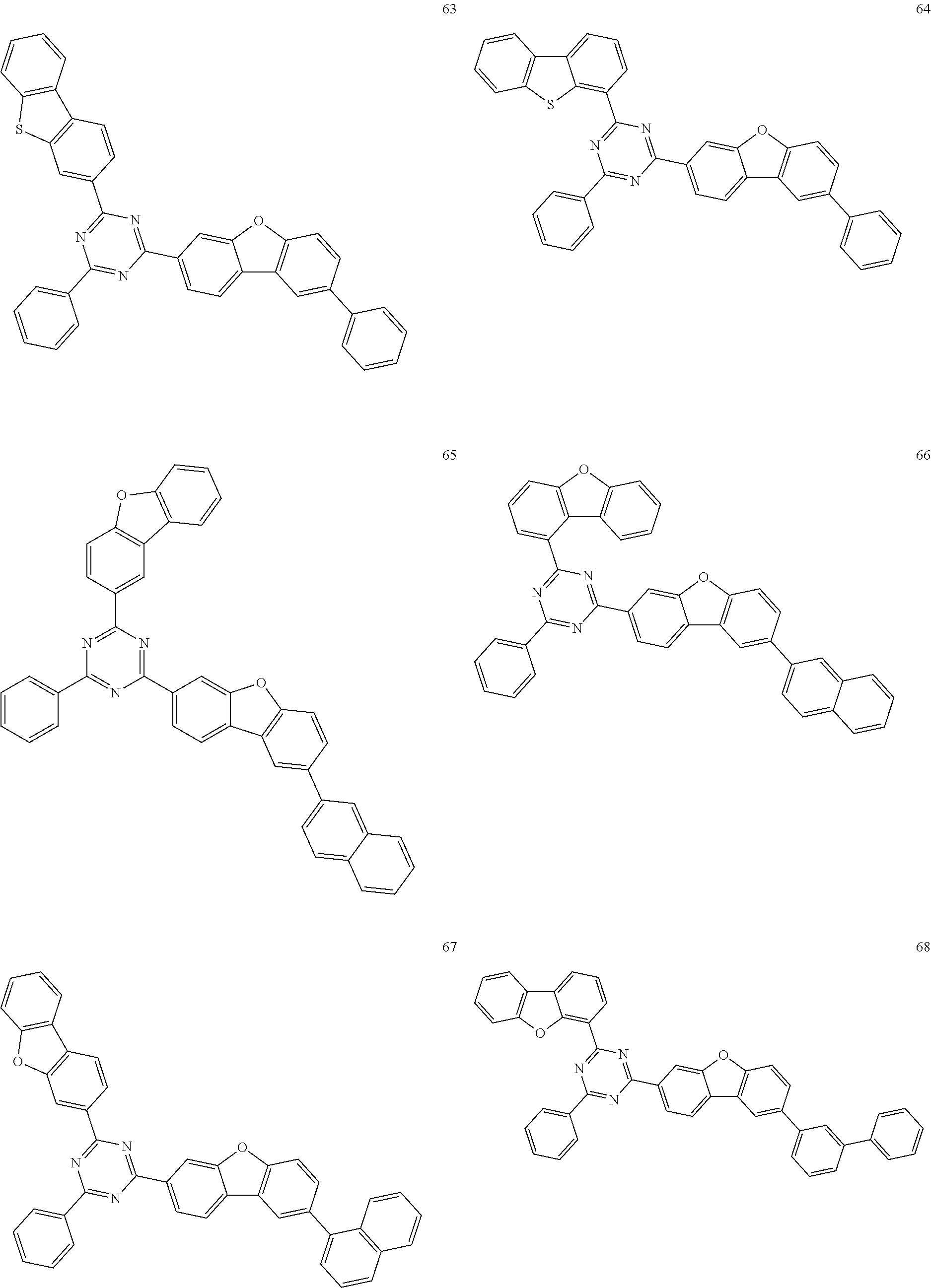 [SMILES: c1ccc(-c2ccc3oc4cc(-c5nc(-c6ccccc6)nc(-c6ccc7c(c6)sc6ccccc67)n5)ccc4c3c2)cc1 ; c1ccc(-c2ccc3oc4cc(-c5nc(-c6ccccc6)nc(-c6cccc7c6sc6ccccc67)n5)ccc4c3c2)cc1 ; c1ccc(-c2nc(-c3ccc4c(c3)oc3ccc(-c5ccc6ccccc6c5)cc34)nc(-c3ccc4oc5ccccc5c4c3)n2)cc1 ; c1ccc(-c2nc(-c3ccc4c(c3)oc3ccc(-c5ccc6ccccc6c5)cc34)nc(-c3cccc4oc5ccccc5c34)n2)cc1 ; c1ccc(-c2nc(-c3ccc4c(c3)oc3ccccc34)nc(-c3ccc4c(c3)oc3ccc(-c5cccc6ccccc56)cc34)n2)cc1 ; c1ccc(-c2cccc(-c3ccc4oc5cc(-c6nc(-c7ccccc7)nc(-c7cccc8c7oc7ccccc78)n6)ccc5c4c3)c2)cc1]

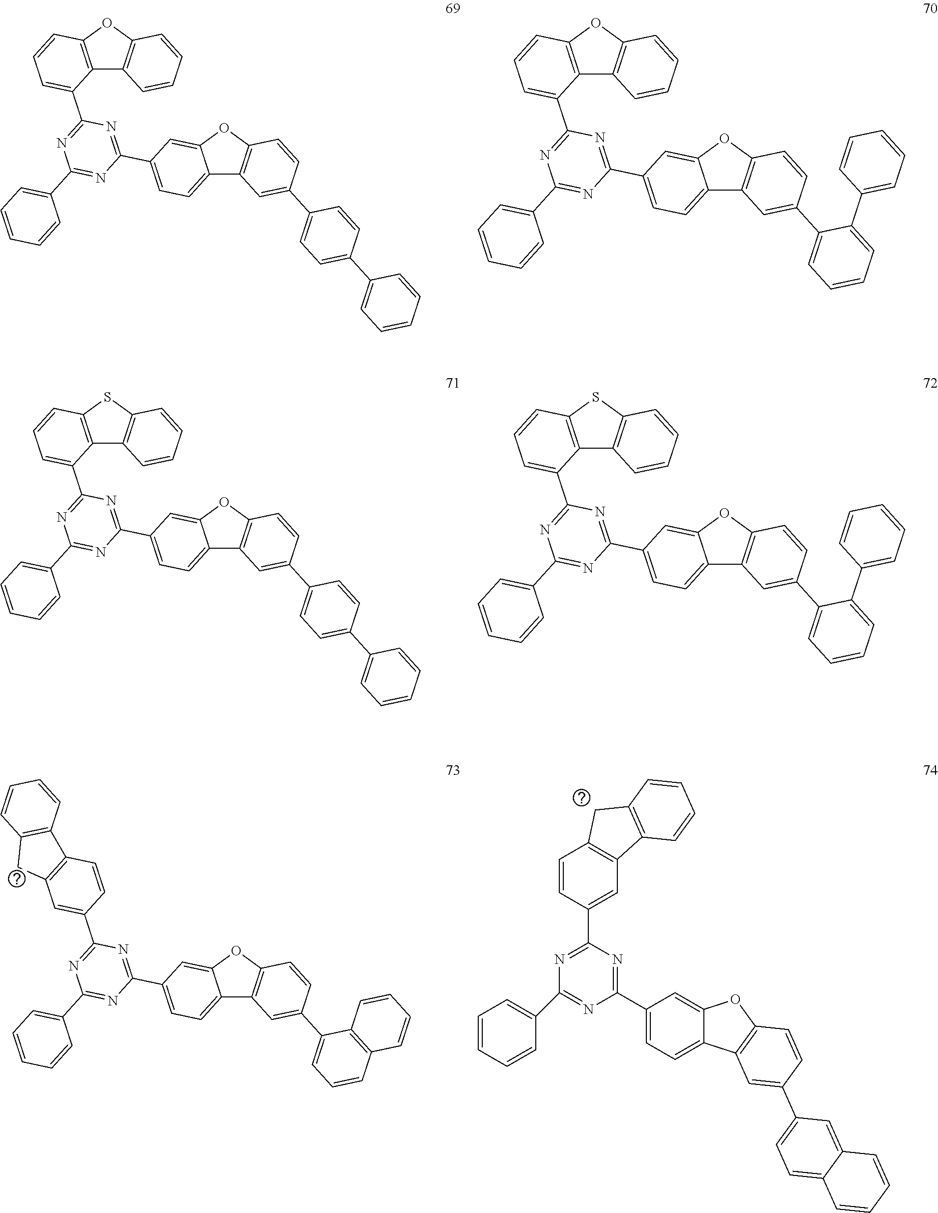

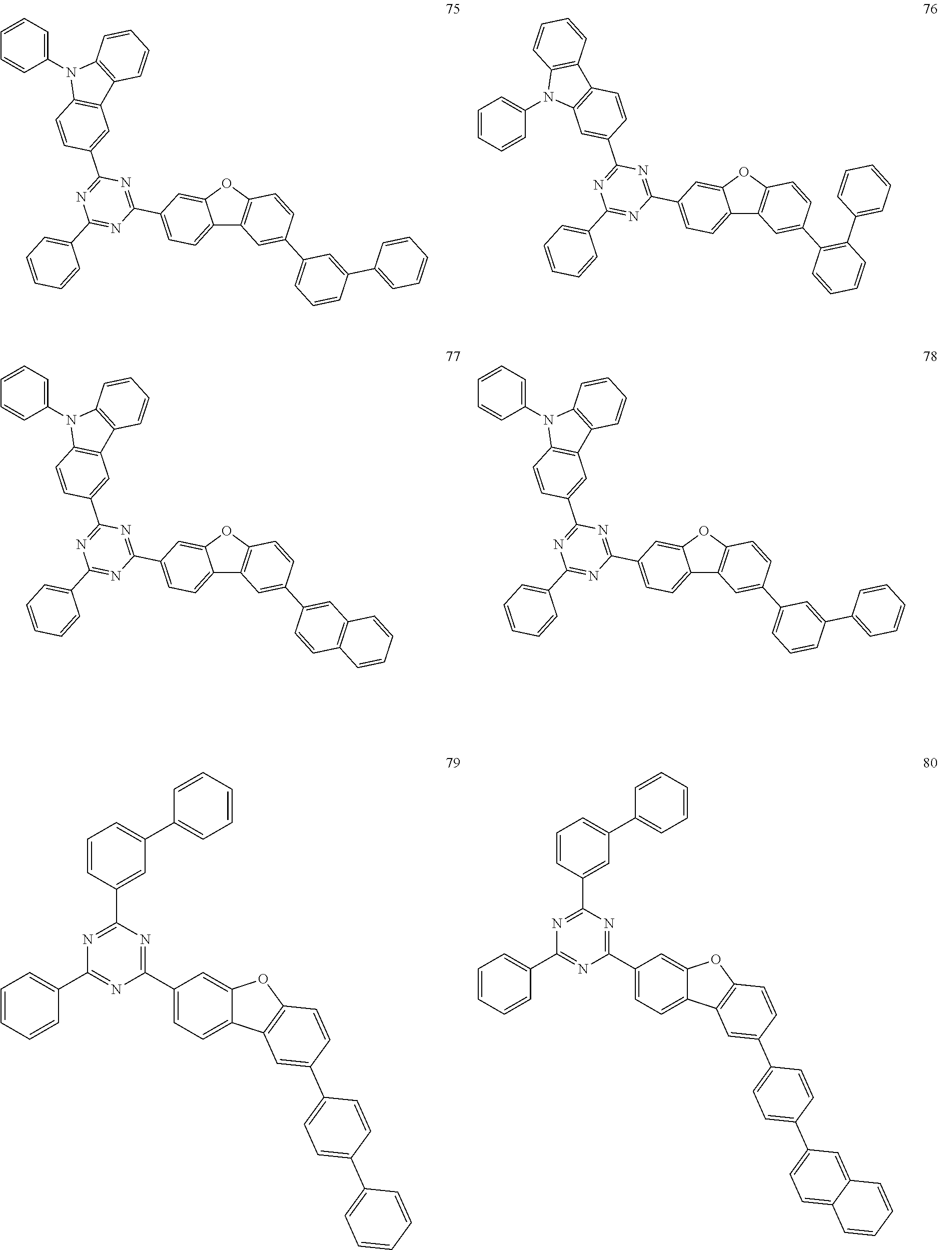

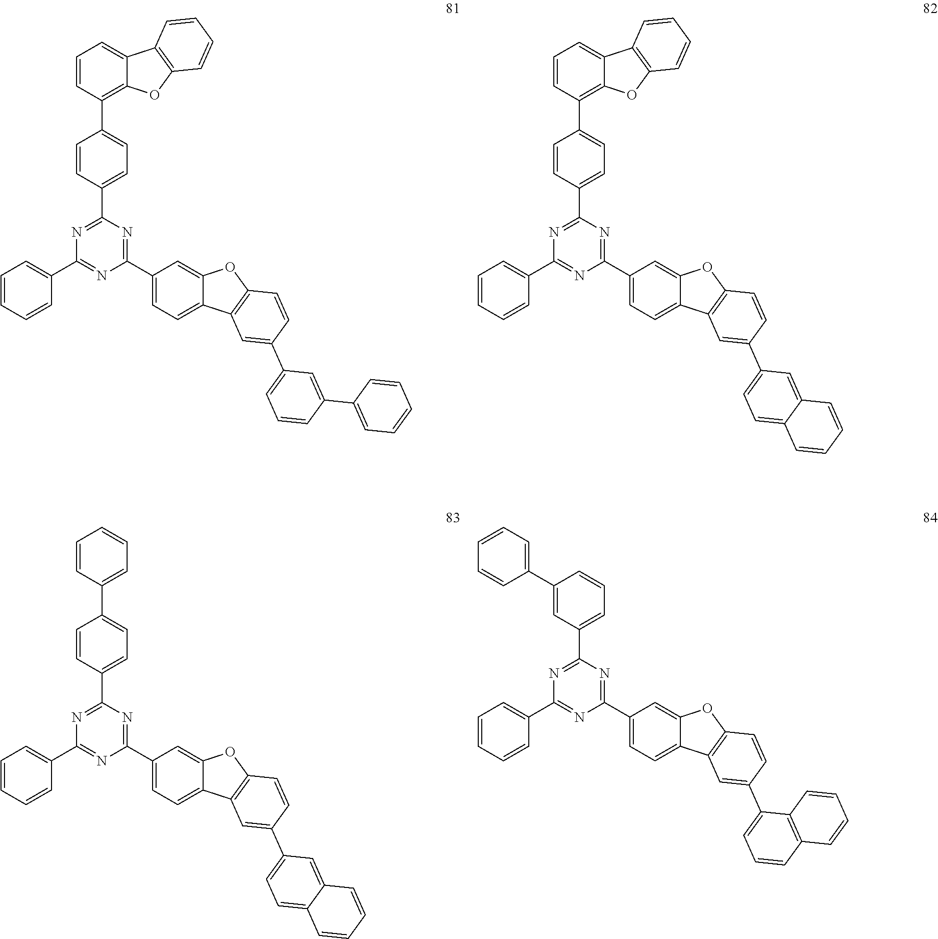 [SMILES: c1ccc(-c2cccc(-c3ccc4oc5cc(-c6nc(-c7ccccc7)nc(-c7ccc(-c8cccc9c8oc8ccccc89)cc7)n6)ccc5c4c3)c2)cc1 ; c1ccc(-c2nc(-c3ccc(-c4cccc5c4oc4ccccc45)cc3)nc(-c3ccc4c(c3)oc3ccc(-c5ccc6ccccc6c5)cc34)n2)cc1 ; c1ccc(-c2ccc(-c3nc(-c4ccccc4)nc(-c4ccc5c(c4)oc4ccc(-c6ccc7ccccc7c6)cc45)n3)cc2)cc1 ; c1ccc(-c2cccc(-c3nc(-c4ccccc4)nc(-c4ccc5c(c4)oc4ccc(-c6cccc7ccccc67)cc45)n3)c2)cc1]

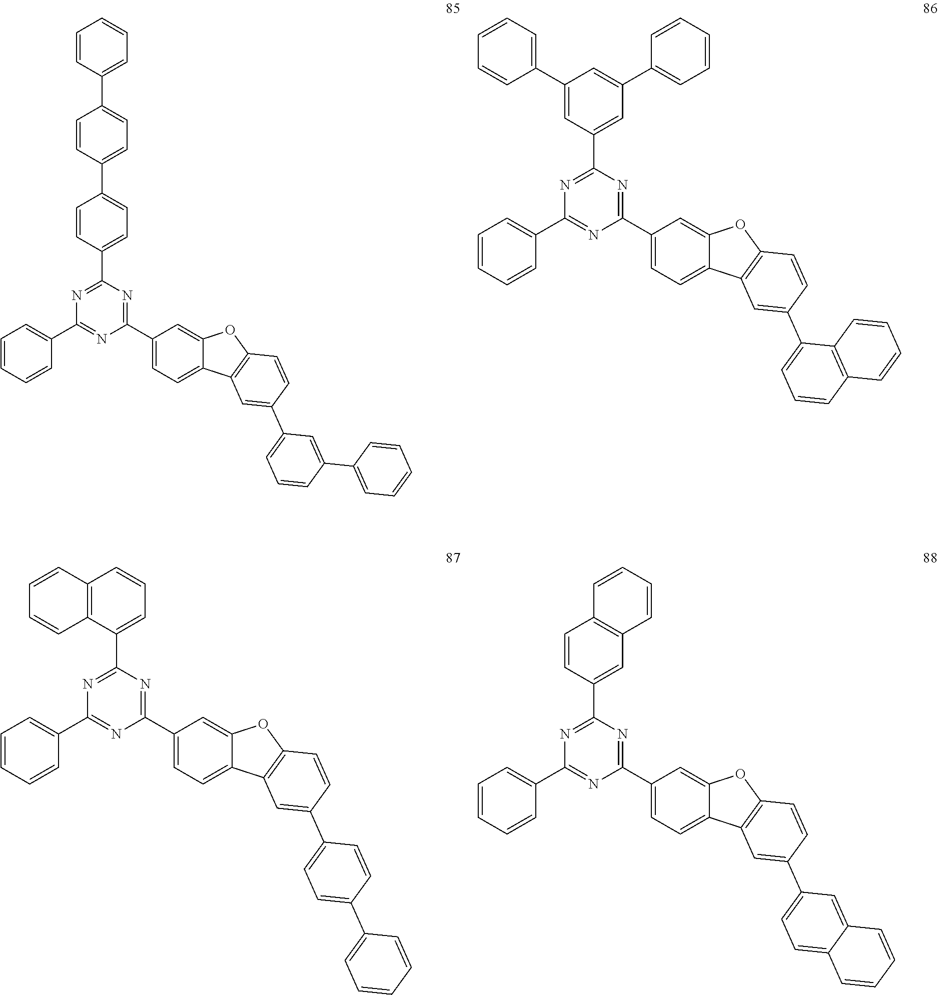 [SMILES: c1ccc(-c2ccc(-c3ccc(-c4nc(-c5ccccc5)nc(-c5ccc6c(c5)oc5ccc(-c7cccc(-c8ccccc8)c7)cc56)n4)cc3)cc2)cc1 ; c1ccc(-c2cc(-c3ccccc3)cc(-c3nc(-c4ccccc4)nc(-c4ccc5c(c4)oc4ccc(-c6cccc7ccccc67)cc45)n3)c2)cc1 ; c1ccc(-c2ccc(-c3ccc4oc5cc(-c6nc(-c7ccccc7)nc(-c7cccc8ccccc78)n6)ccc5c4c3)cc2)cc1 ; c1ccc(-c2nc(-c3ccc4ccccc4c3)nc(-c3ccc4c(c3)oc3ccc(-c5ccc6ccccc6c5)cc34)n2)cc1]

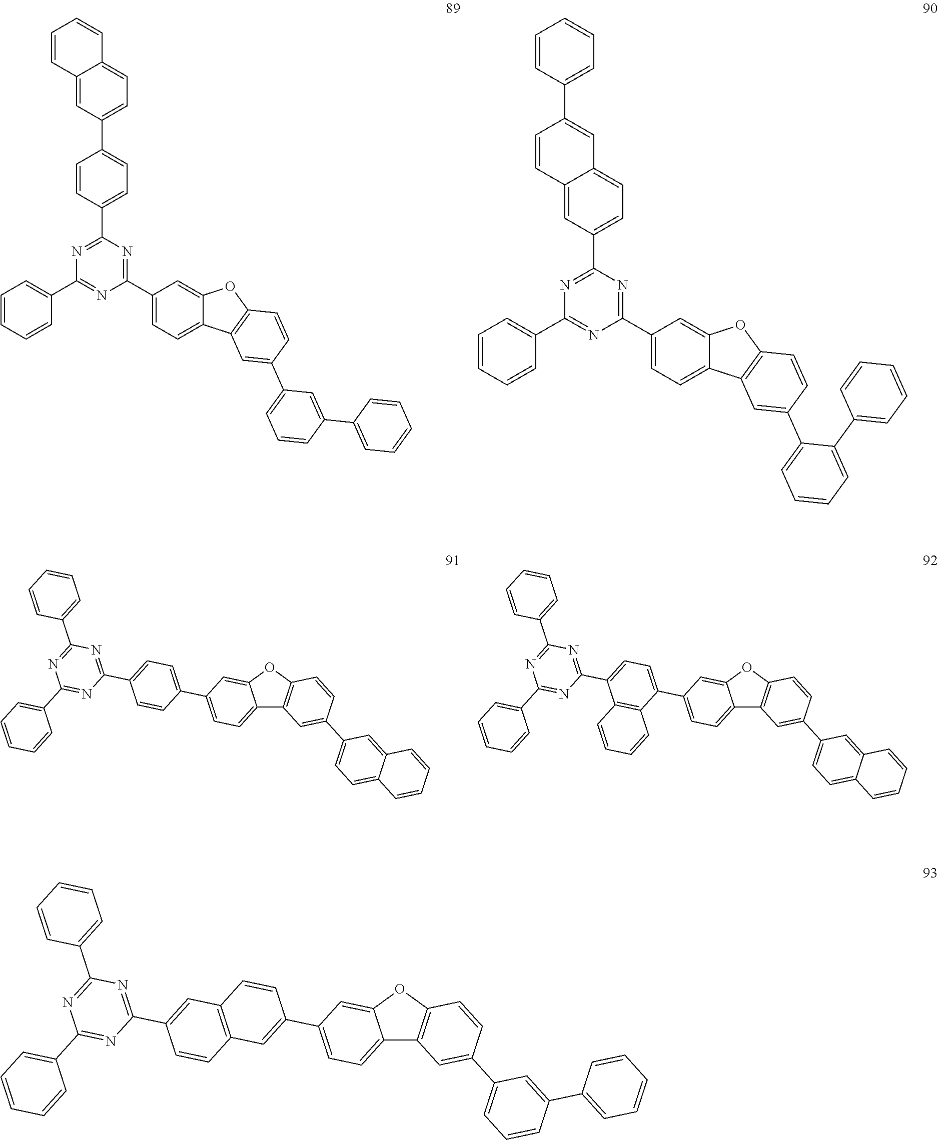 [SMILES: c1ccc(-c2cccc(-c3ccc4oc5cc(-c6nc(-c7ccccc7)nc(-c7ccc(-c8ccc9ccccc9c8)cc7)n6)ccc5c4c3)c2)cc1 ; c1ccc(-c2ccc3cc(-c4nc(-c5ccccc5)nc(-c5ccc6c(c5)oc5ccc(-c7ccccc7-c7ccccc7)cc56)n4)ccc3c2)cc1 ; c1ccc(-c2nc(-c3ccccc3)nc(-c3ccc(-c4ccc5c(c4)oc4ccc(-c6ccc7ccccc7c6)cc45)cc3)n2)cc1 ; c1ccc(-c2nc(-c3ccccc3)nc(-c3ccc(-c4ccc5c(c4)oc4ccc(-c6ccc7ccccc7c6)cc45)c4ccccc34)n2)cc1 ; c1ccc(-c2cccc(-c3ccc4oc5cc(-c6ccc7cc(-c8nc(-c9ccccc9)nc(-c9ccccc9)n8)ccc7c6)ccc5c4c3)c2)cc1]

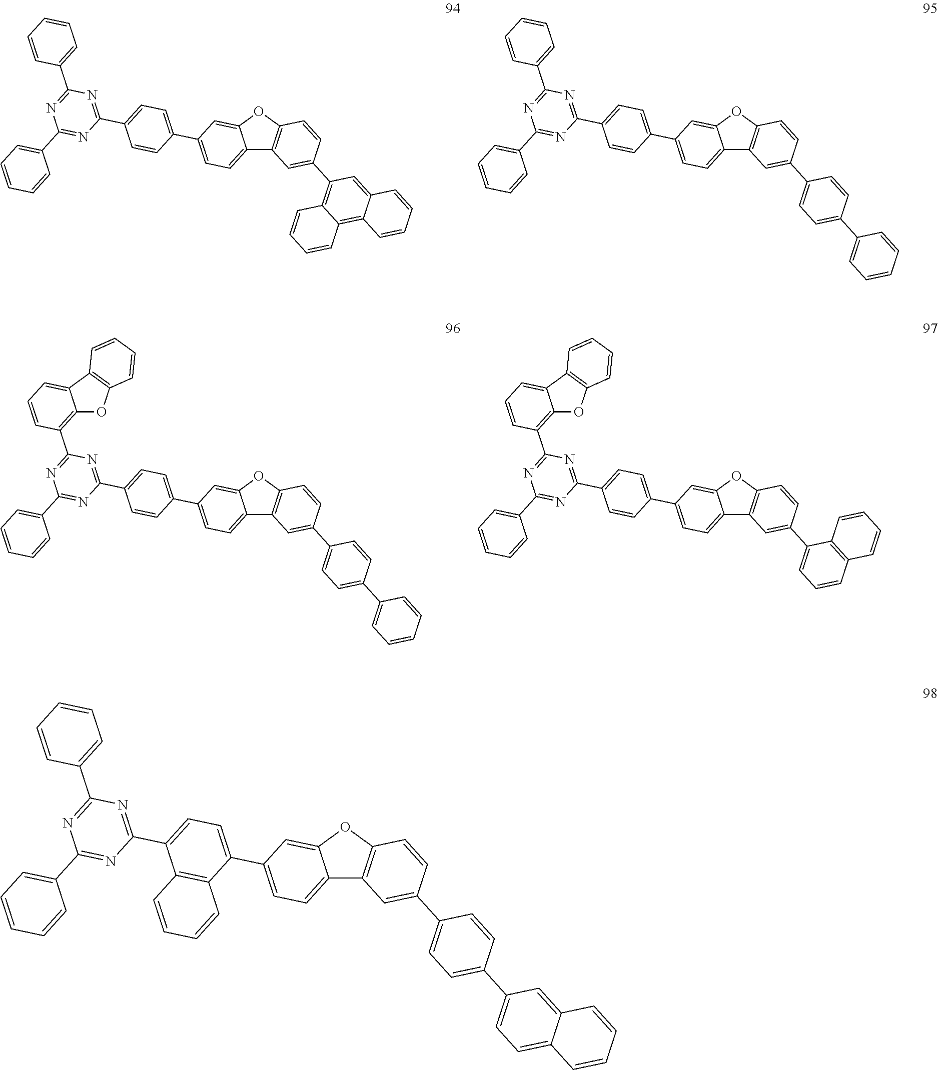 [SMILES: c1ccc(-c2nc(-c3ccccc3)nc(-c3ccc(-c4ccc5c(c4)oc4ccc(-c6cc7ccccc7c7ccccc67)cc45)cc3)n2)cc1 ; c1ccc(-c2ccc(-c3ccc4oc5cc(-c6ccc(-c7nc(-c8ccccc8)nc(-c8ccccc8)n7)cc6)ccc5c4c3)cc2)cc1 ; c1ccc(-c2ccc(-c3ccc4oc5cc(-c6ccc(-c7nc(-c8ccccc8)nc(-c8cccc9c8oc8ccccc89)n7)cc6)ccc5c4c3)cc2)cc1 ; c1ccc(-c2nc(-c3ccc(-c4ccc5c(c4)oc4ccc(-c6cccc7ccccc67)cc45)cc3)nc(-c3cccc4c3oc3ccccc34)n2)cc1 ; c1ccc(-c2nc(-c3ccccc3)nc(-c3ccc(-c4ccc5c(c4)oc4ccc(-c6ccc(-c7ccc8ccccc8c7)cc6)cc45)c4ccccc34)n2)cc1]

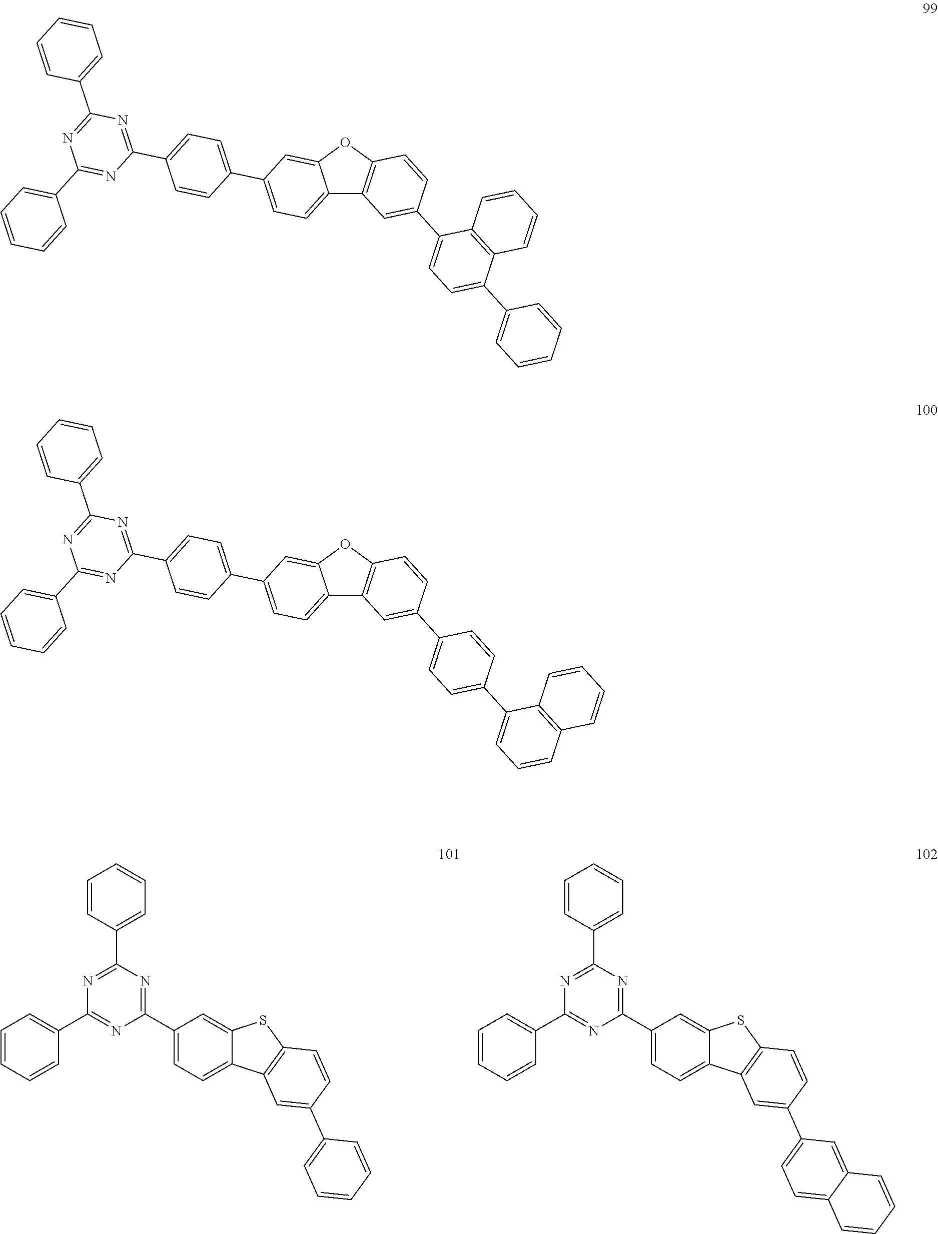 [SMILES: c1ccc(-c2nc(-c3ccccc3)nc(-c3ccc(-c4ccc5c(c4)oc4ccc(-c6ccc(-c7ccccc7)c7ccccc67)cc45)cc3)n2)cc1 ; c1ccc(-c2nc(-c3ccccc3)nc(-c3ccc(-c4ccc5c(c4)oc4ccc(-c6ccc(-c7cccc8ccccc78)cc6)cc45)cc3)n2)cc1 ; c1ccc(-c2ccc3sc4cc(-c5nc(-c6ccccc6)nc(-c6ccccc6)n5)ccc4c3c2)cc1 ; c1ccc(-c2nc(-c3ccccc3)nc(-c3ccc4c(c3)sc3ccc(-c5ccc6ccccc6c5)cc34)n2)cc1]

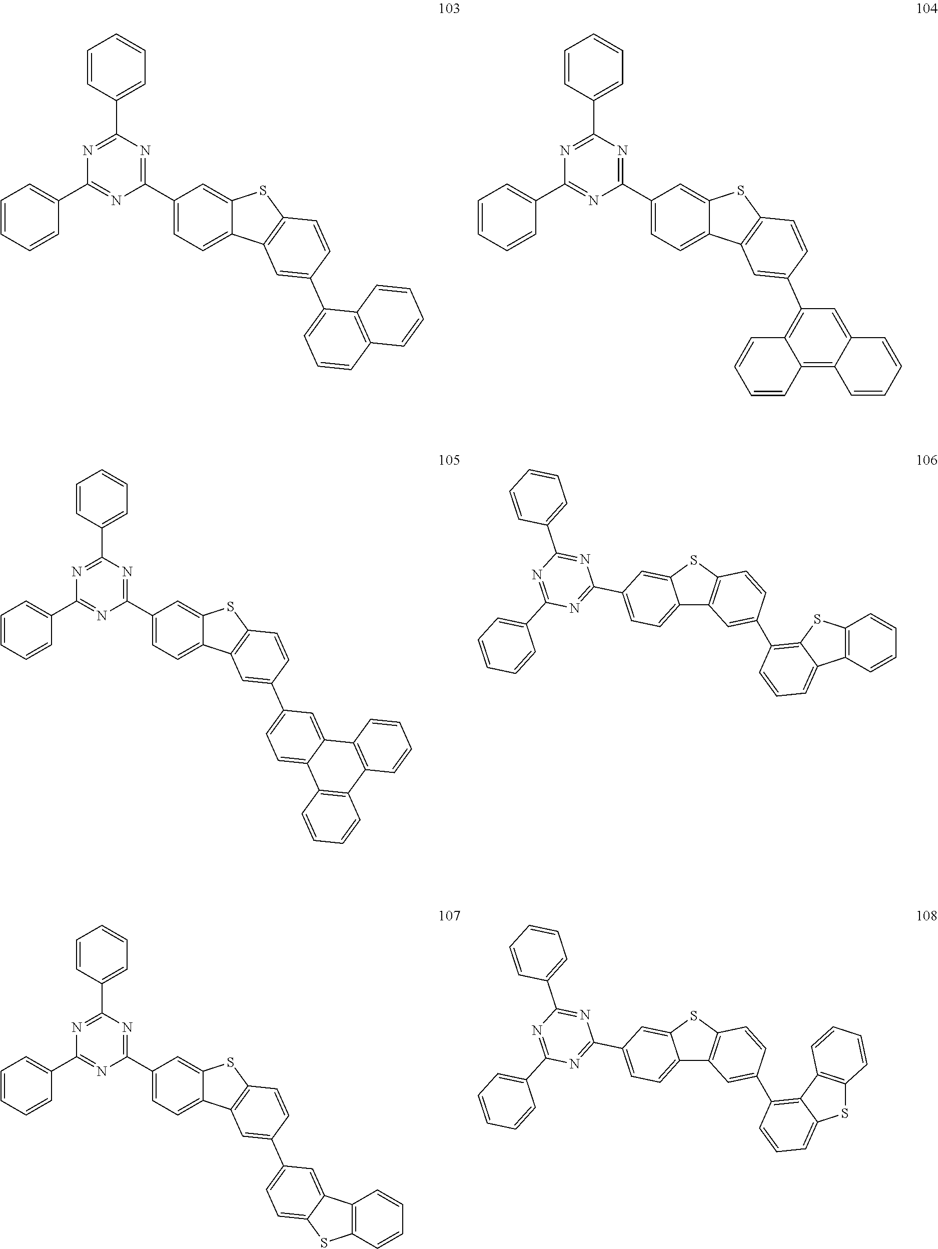 [SMILES: c1ccc(-c2nc(-c3ccccc3)nc(-c3ccc4c(c3)sc3ccc(-c5cccc6ccccc56)cc34)n2)cc1 ; c1ccc(-c2nc(-c3ccccc3)nc(-c3ccc4c(c3)sc3ccc(-c5cc6ccccc6c6ccccc56)cc34)n2)cc1 ; c1ccc(-c2nc(-c3ccccc3)nc(-c3ccc4c(c3)sc3ccc(-c5ccc6c7ccccc7c7ccccc7c6c5)cc34)n2)cc1 ; c1ccc(-c2nc(-c3ccccc3)nc(-c3ccc4c(c3)sc3ccc(-c5cccc6c5sc5ccccc56)cc34)n2)cc1 ; c1ccc(-c2nc(-c3ccccc3)nc(-c3ccc4c(c3)sc3ccc(-c5ccc6sc7ccccc7c6c5)cc34)n2)cc1 ; c1ccc(-c2nc(-c3ccccc3)nc(-c3ccc4c(c3)sc3ccc(-c5cccc6sc7ccccc7c56)cc34)n2)cc1]

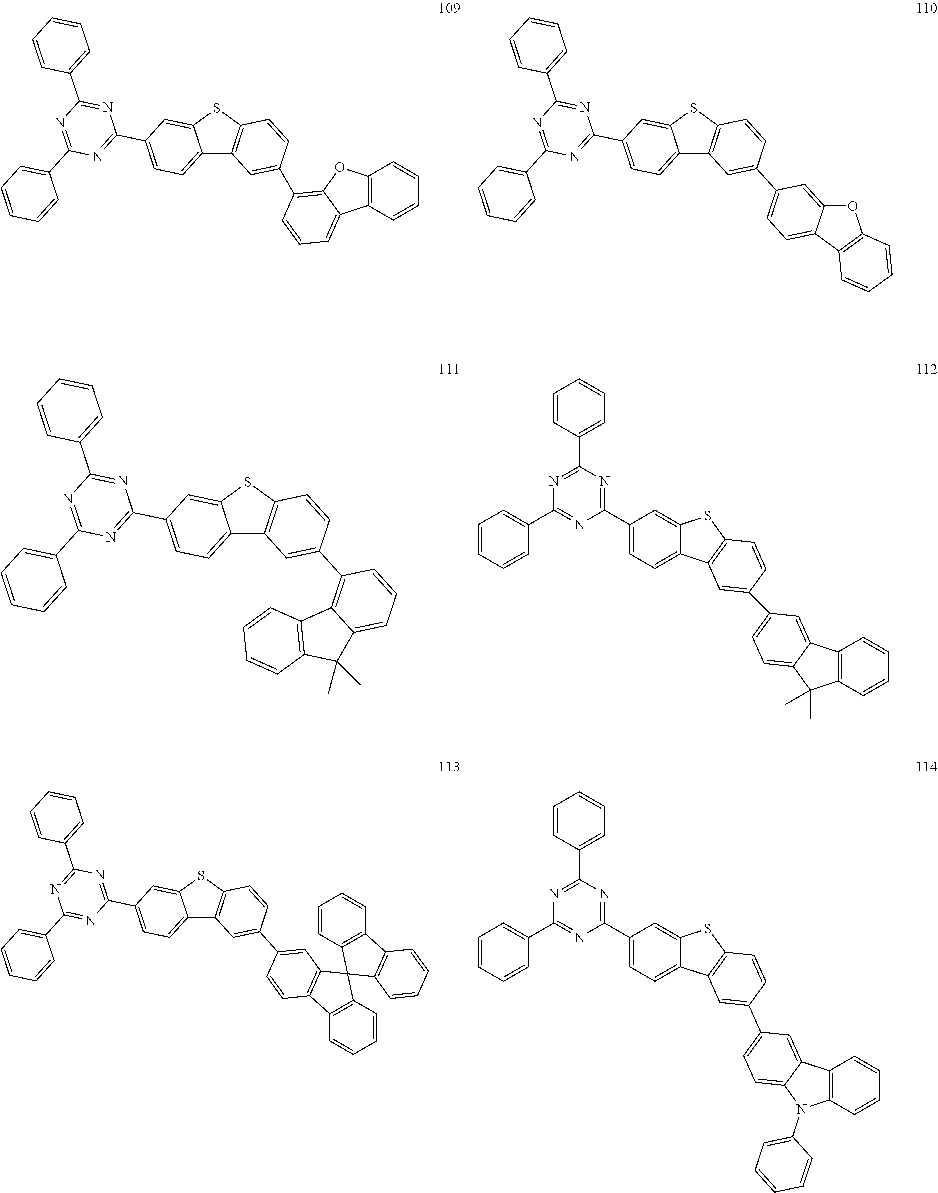

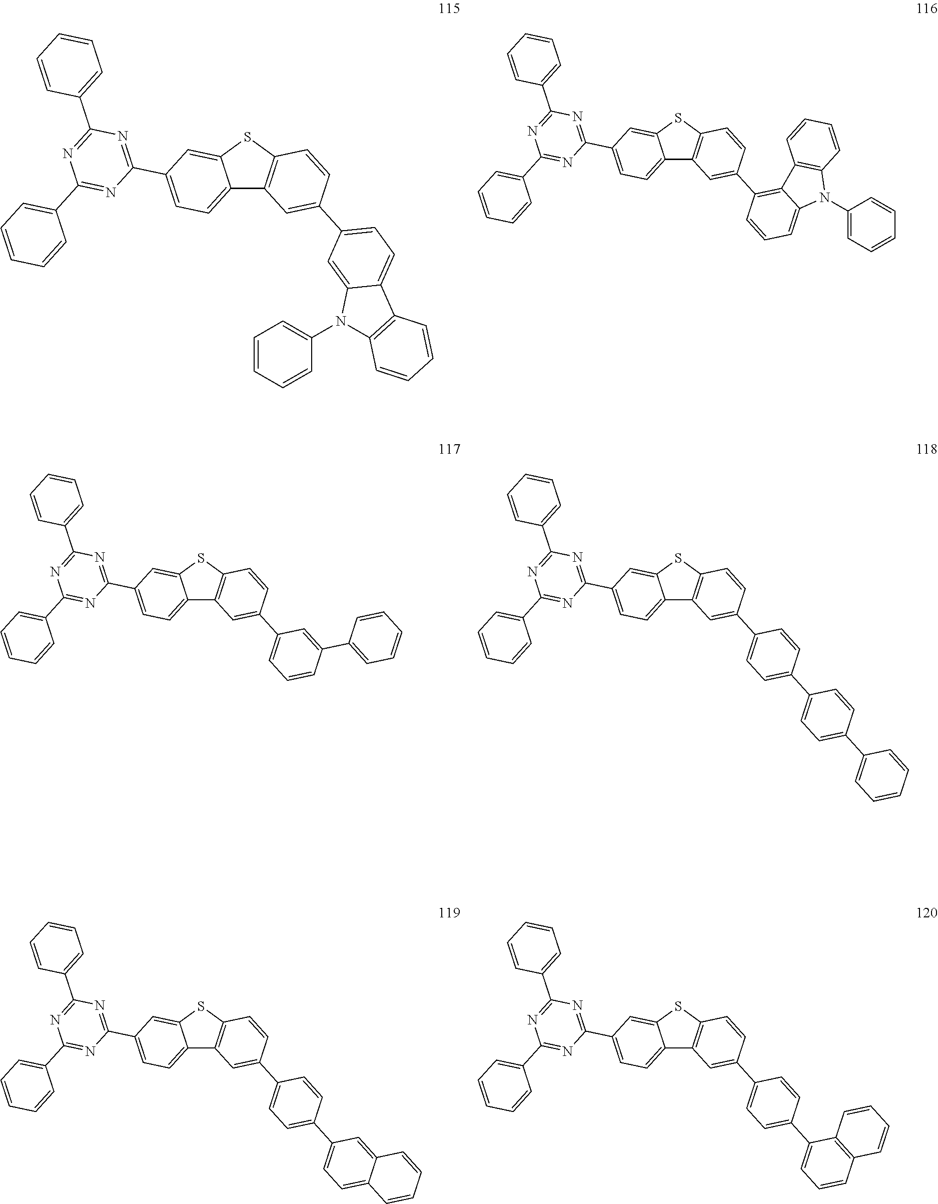

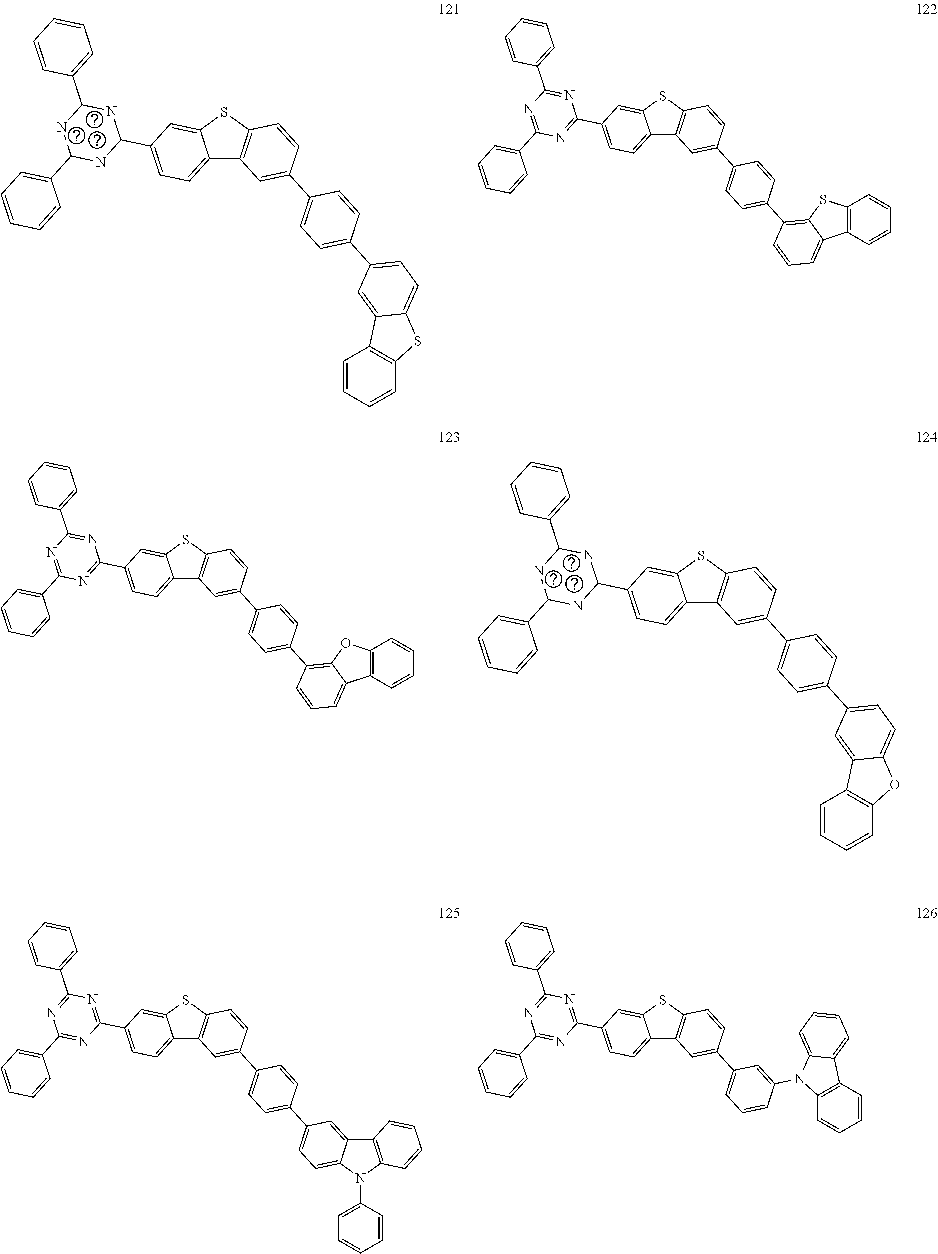

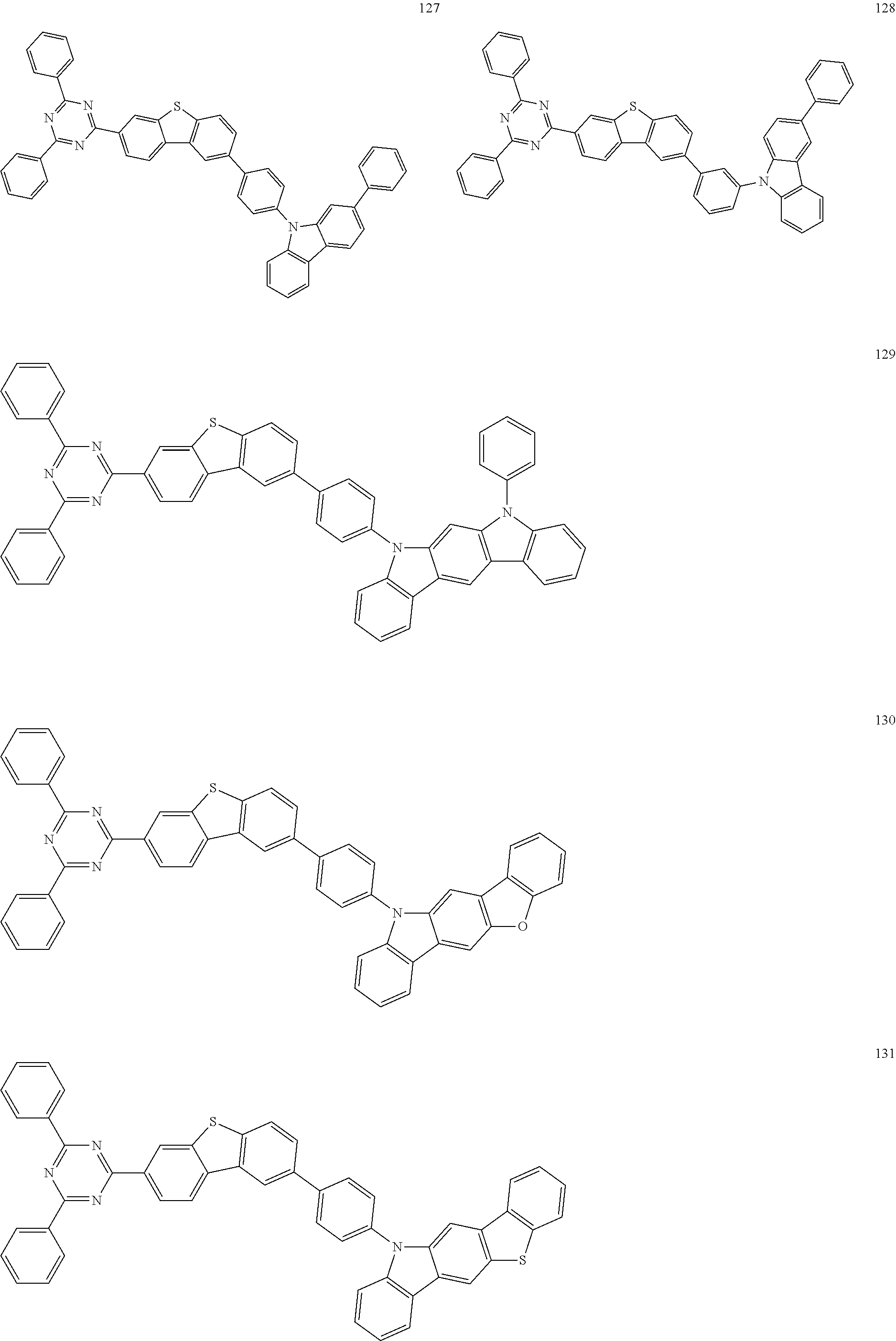

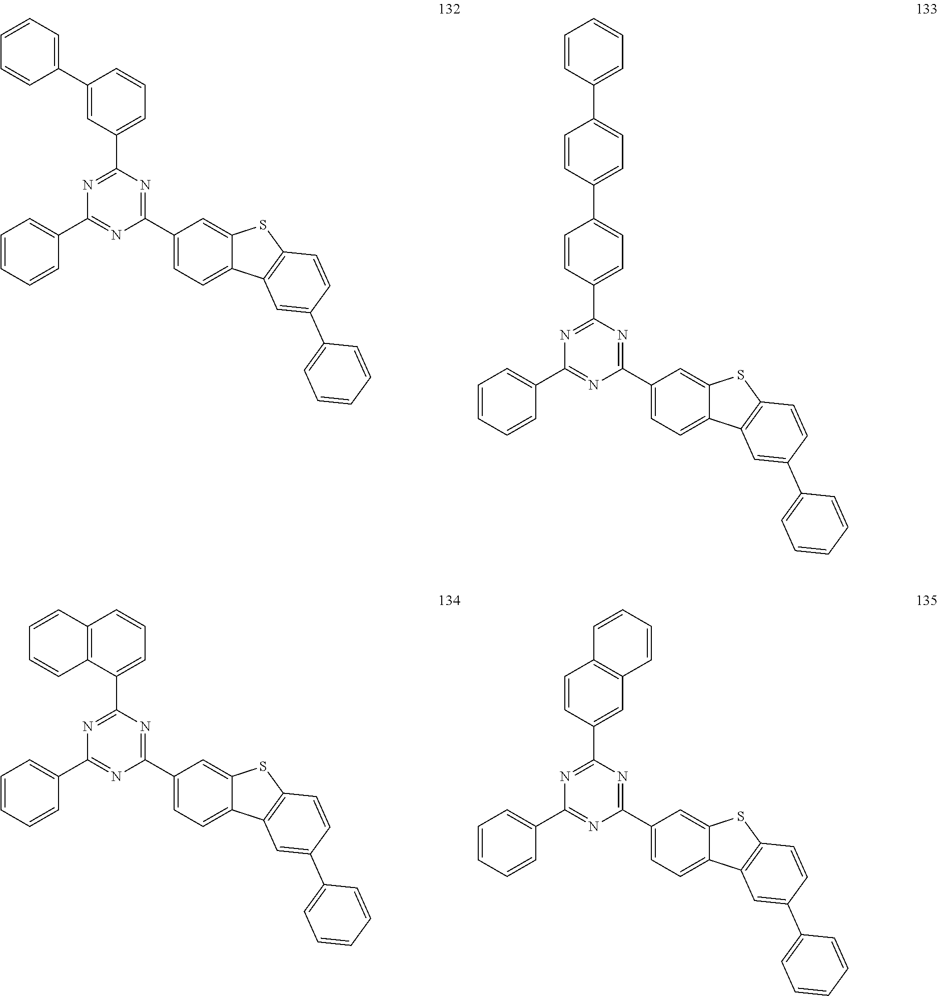 [SMILES: c1ccc(-c2cccc(-c3nc(-c4ccccc4)nc(-c4ccc5c(c4)sc4ccc(-c6ccccc6)cc45)n3)c2)cc1 ; c1ccc(-c2ccc(-c3ccc(-c4nc(-c5ccccc5)nc(-c5ccc6c(c5)sc5ccc(-c7ccccc7)cc56)n4)cc3)cc2)cc1 ; c1ccc(-c2ccc3sc4cc(-c5nc(-c6ccccc6)nc(-c6cccc7ccccc67)n5)ccc4c3c2)cc1 ; c1ccc(-c2ccc3sc4cc(-c5nc(-c6ccccc6)nc(-c6ccc7ccccc7c6)n5)ccc4c3c2)cc1]

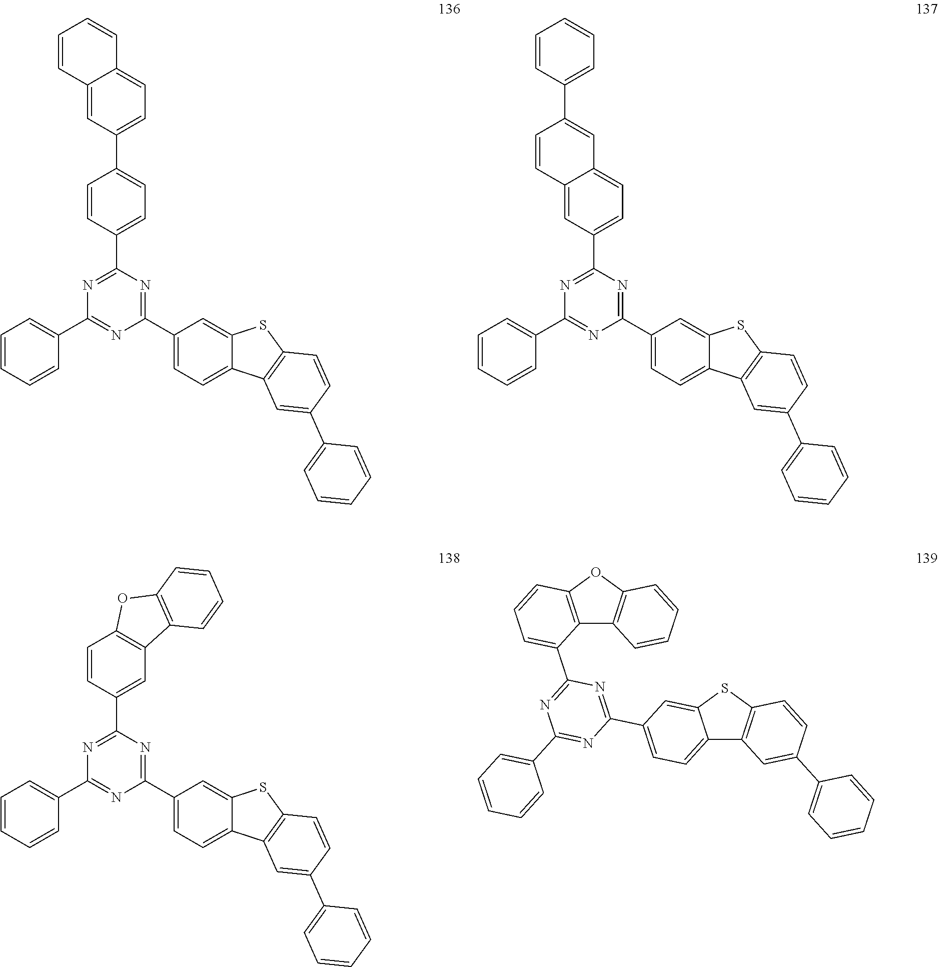 [SMILES: c1ccc(-c2ccc3sc4cc(-c5nc(-c6ccccc6)nc(-c6ccc(-c7ccc8ccccc8c7)cc6)n5)ccc4c3c2)cc1 ; c1ccc(-c2ccc3cc(-c4nc(-c5ccccc5)nc(-c5ccc6c(c5)sc5ccc(-c7ccccc7)cc56)n4)ccc3c2)cc1 ; c1ccc(-c2ccc3sc4cc(-c5nc(-c6ccccc6)nc(-c6ccc7oc8ccccc8c7c6)n5)ccc4c3c2)cc1 ; c1ccc(-c2ccc3sc4cc(-c5nc(-c6ccccc6)nc(-c6cccc7oc8ccccc8c67)n5)ccc4c3c2)cc1]

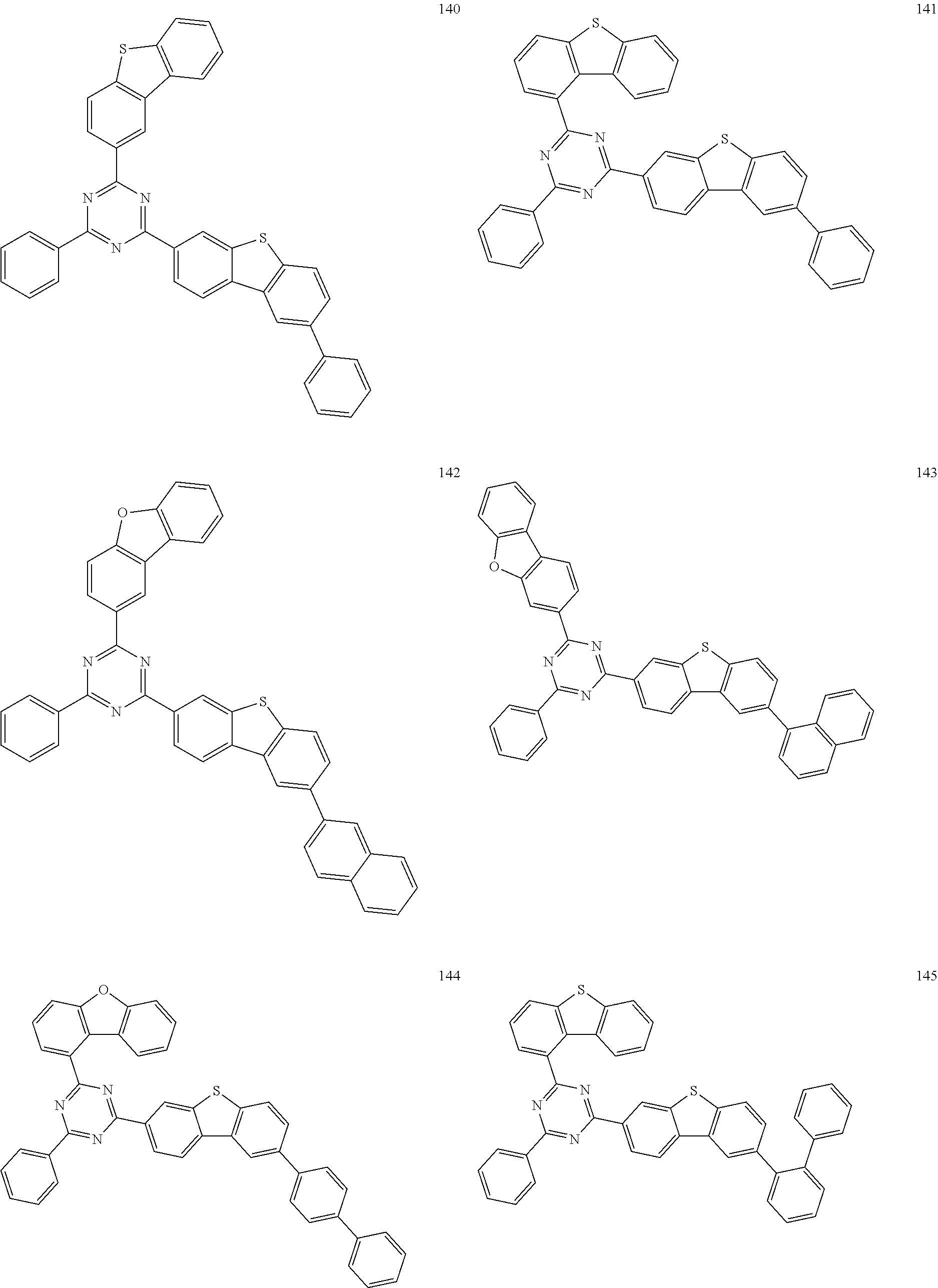 [SMILES: c1ccc(-c2ccc3sc4cc(-c5nc(-c6ccccc6)nc(-c6ccc7sc8ccccc8c7c6)n5)ccc4c3c2)cc1 ; c1ccc(-c2ccc3sc4cc(-c5nc(-c6ccccc6)nc(-c6cccc7sc8ccccc8c67)n5)ccc4c3c2)cc1 ; c1ccc(-c2nc(-c3ccc4c(c3)sc3ccc(-c5ccc6ccccc6c5)cc34)nc(-c3ccc4oc5ccccc5c4c3)n2)cc1 ; c1ccc(-c2nc(-c3ccc4c(c3)oc3ccccc34)nc(-c3ccc4c(c3)sc3ccc(-c5cccc6ccccc56)cc34)n2)cc1 ; c1ccc(-c2ccc(-c3ccc4sc5cc(-c6nc(-c7ccccc7)nc(-c7cccc8oc9ccccc9c78)n6)ccc5c4c3)cc2)cc1 ; c1ccc(-c2nc(-c3ccc4c(c3)sc3ccc(-c5ccccc5-c5ccccc5)cc34)nc(-c3cccc4sc5ccccc5c34)n2)cc1]

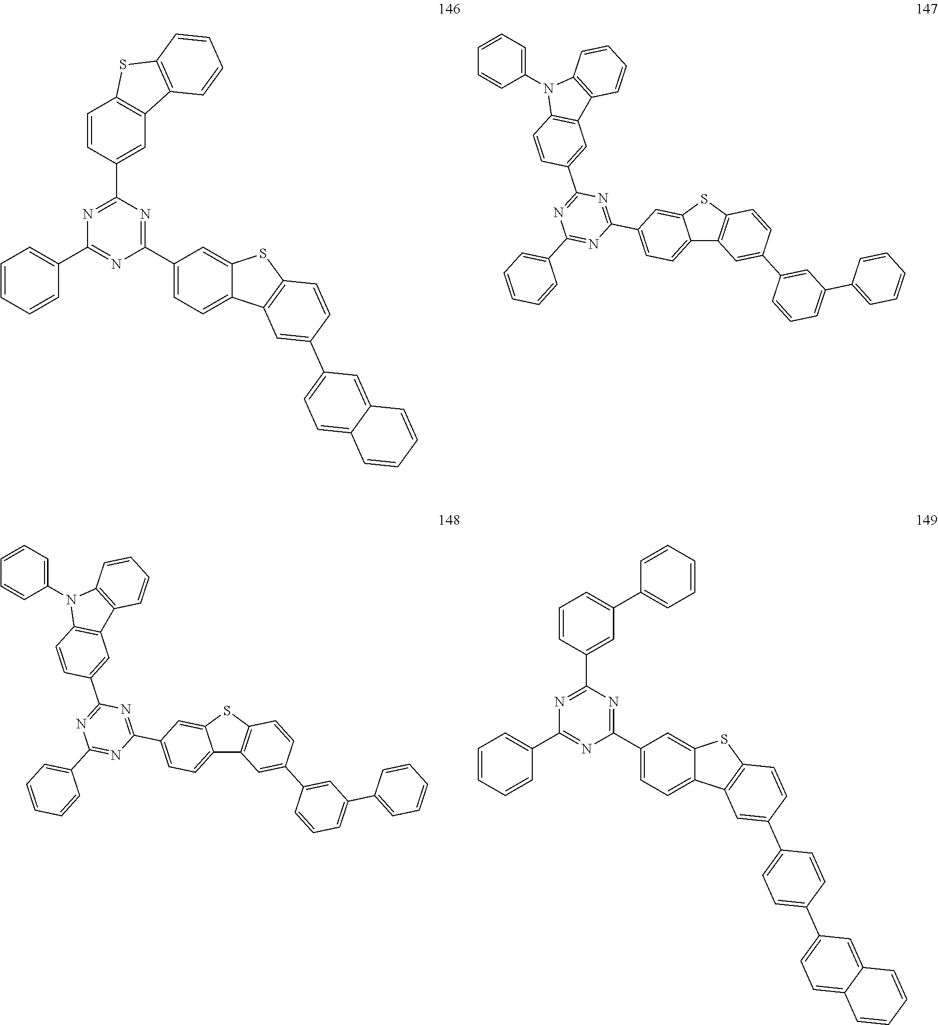 [SMILES: c1ccc(-c2nc(-c3ccc4c(c3)sc3ccc(-c5ccc6ccccc6c5)cc34)nc(-c3ccc4sc5ccccc5c4c3)n2)cc1 ; c1ccc(-c2cccc(-c3ccc4sc5cc(-c6nc(-c7ccccc7)nc(-c7ccc8c(c7)c7ccccc7n8-c7ccccc7)n6)ccc5c4c3)c2)cc1 ; c1ccc(-c2cccc(-c3ccc4sc5cc(-c6nc(-c7ccccc7)nc(-c7ccc8c(c7)c7ccccc7n8-c7ccccc7)n6)ccc5c4c3)c2)cc1 ; c1ccc(-c2cccc(-c3nc(-c4ccccc4)nc(-c4ccc5c(c4)sc4ccc(-c6ccc(-c7ccc8ccccc8c7)cc6)cc45)n3)c2)cc1]

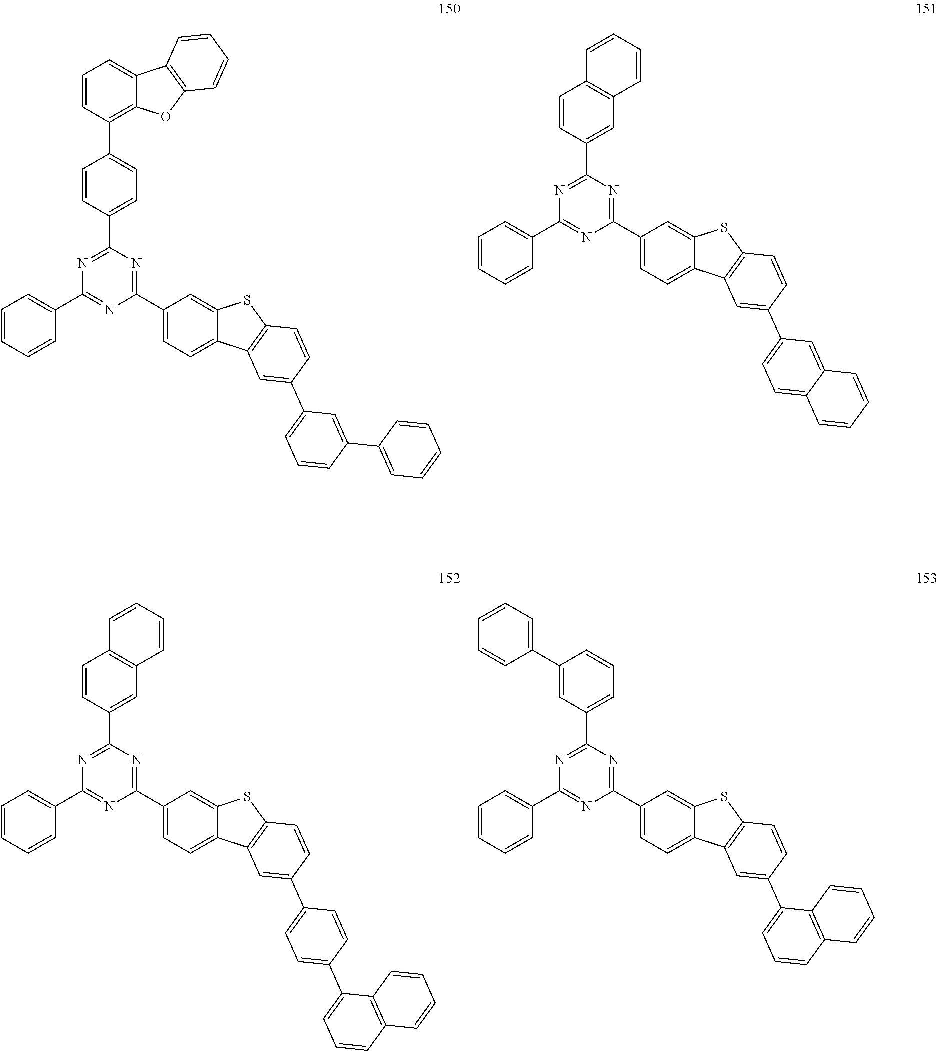 [SMILES: c1ccc(-c2cccc(-c3ccc4sc5cc(-c6nc(-c7ccccc7)nc(-c7ccc(-c8cccc9c8oc8ccccc89)cc7)n6)ccc5c4c3)c2)cc1 ; c1ccc(-c2nc(-c3ccc4ccccc4c3)nc(-c3ccc4c(c3)sc3ccc(-c5ccc6ccccc6c5)cc34)n2)cc1 ; c1ccc(-c2nc(-c3ccc4ccccc4c3)nc(-c3ccc4c(c3)sc3ccc(-c5ccc(-c6cccc7ccccc67)cc5)cc34)n2)cc1 ; c1ccc(-c2cccc(-c3nc(-c4ccccc4)nc(-c4ccc5c(c4)sc4ccc(-c6cccc7ccccc67)cc45)n3)c2)cc1]

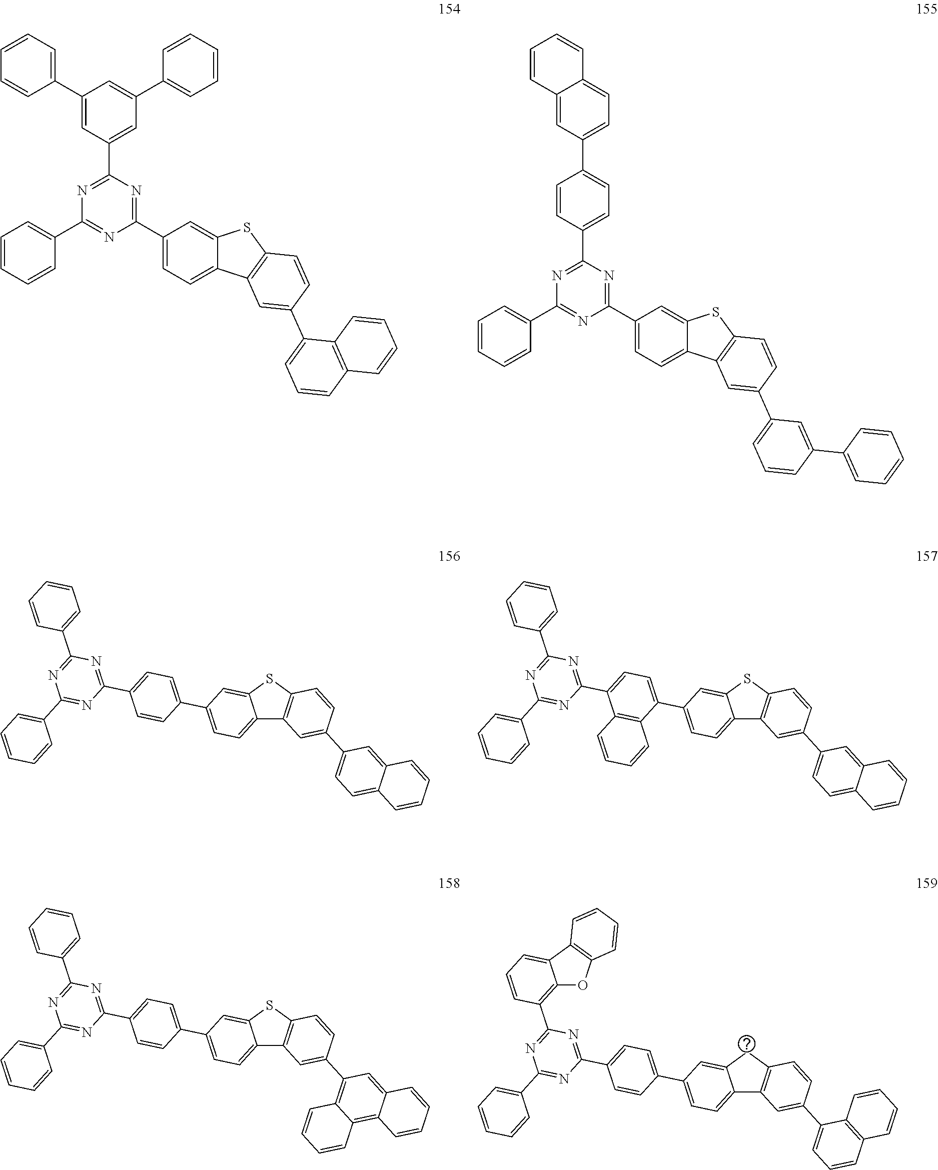

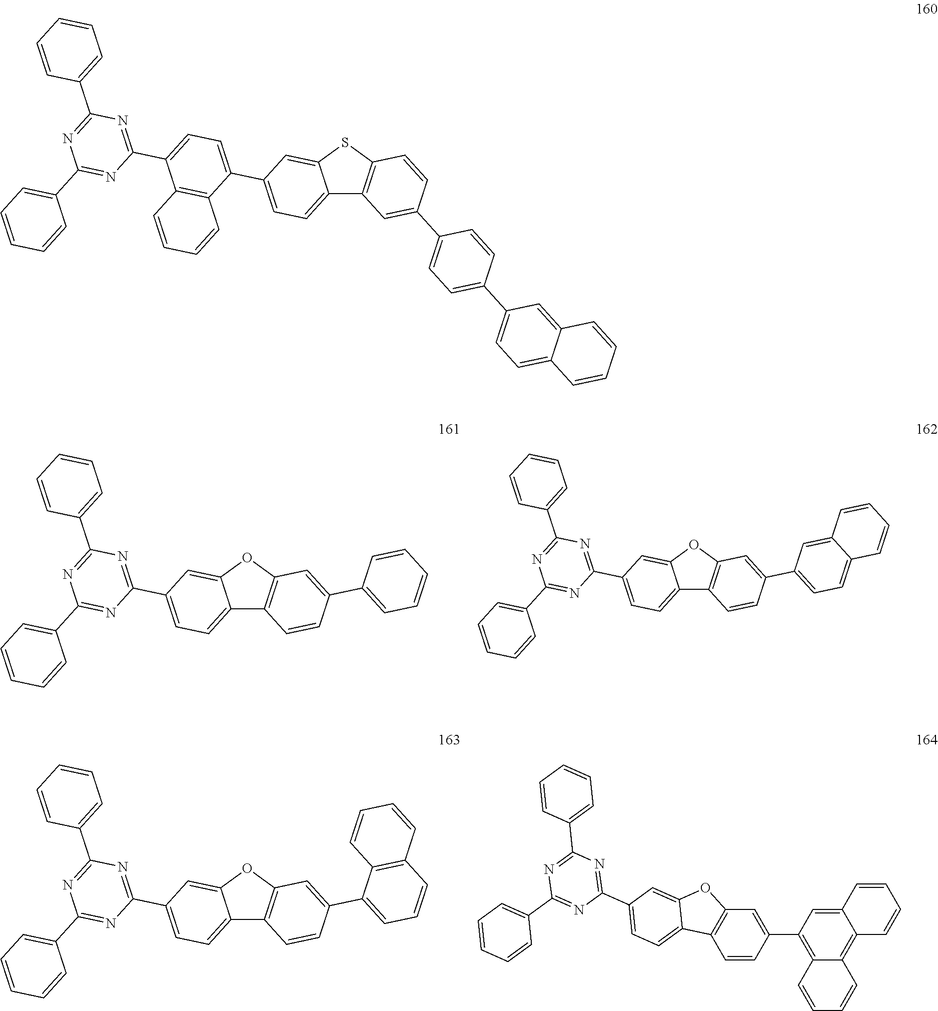 [SMILES: c1ccc(-c2nc(-c3ccccc3)nc(-c3ccc(-c4ccc5c(c4)sc4ccc(-c6ccc(-c7ccc8ccccc8c7)cc6)cc45)c4ccccc34)n2)cc1 ; c1ccc(-c2ccc3c(c2)oc2cc(-c4nc(-c5ccccc5)nc(-c5ccccc5)n4)ccc23)cc1 ; c1ccc(-c2nc(-c3ccccc3)nc(-c3ccc4c(c3)oc3cc(-c5ccc6ccccc6c5)ccc34)n2)cc1 ; c1ccc(-c2nc(-c3ccccc3)nc(-c3ccc4c(c3)oc3cc(-c5cccc6ccccc56)ccc34)n2)cc1 ; c1ccc(-c2nc(-c3ccccc3)nc(-c3ccc4c(c3)oc3cc(-c5cc6ccccc6c6ccccc56)ccc34)n2)cc1]

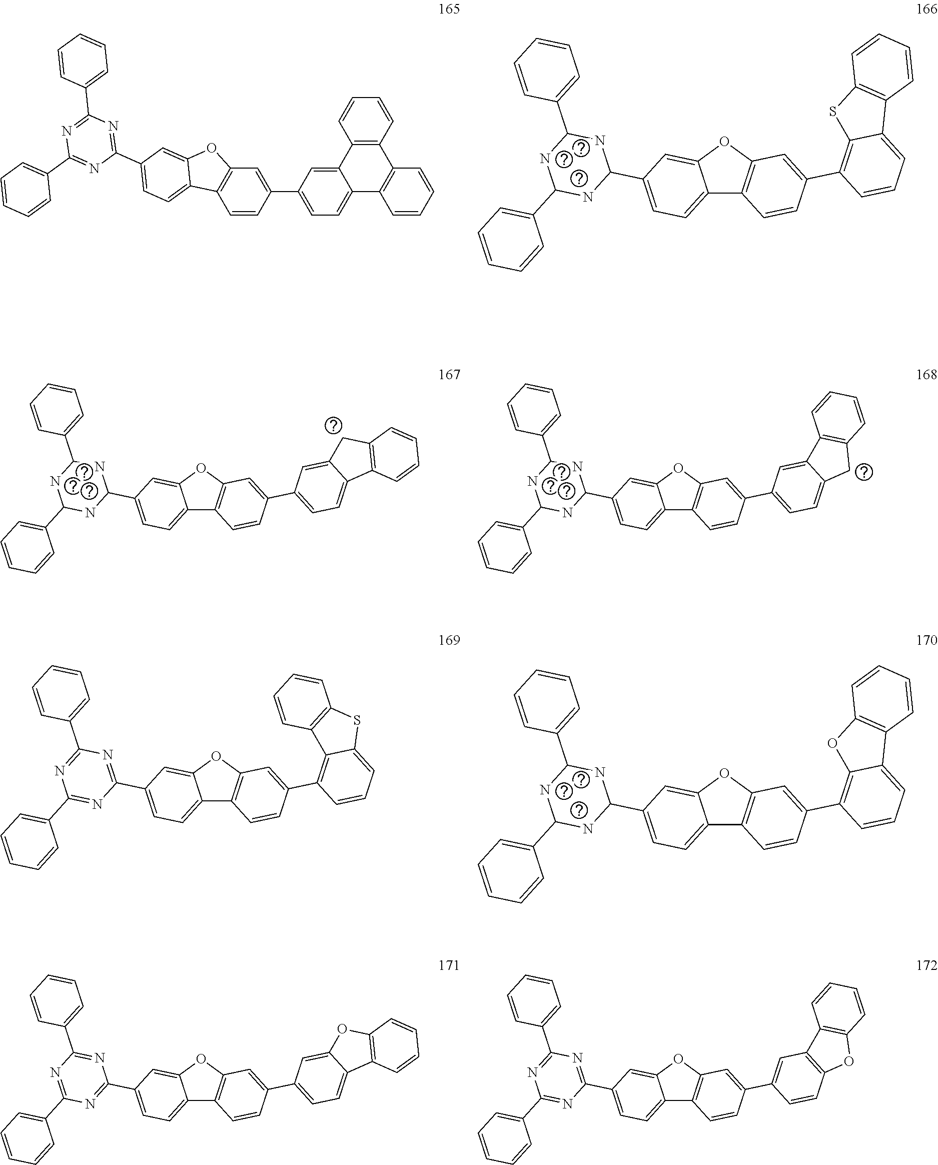

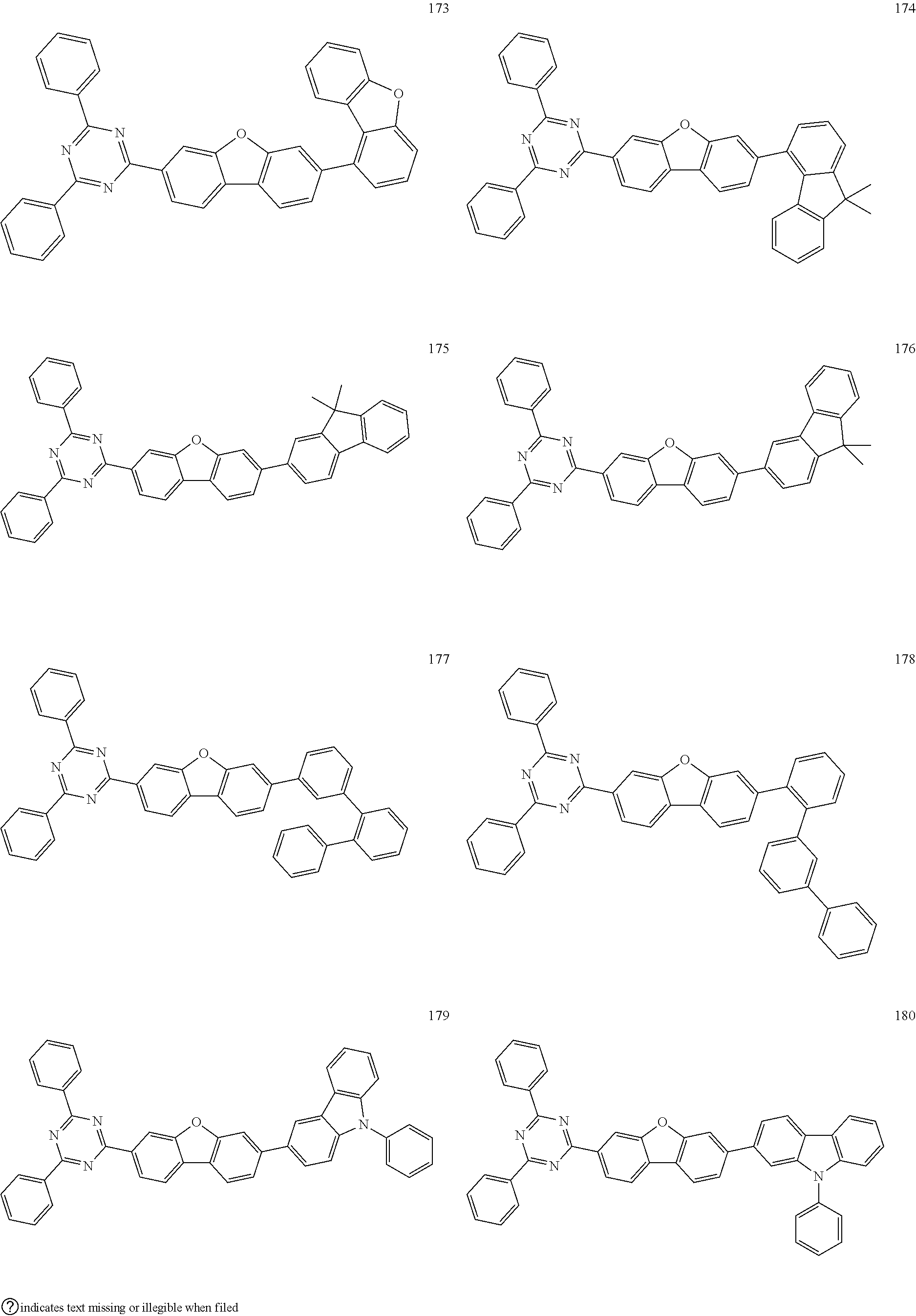

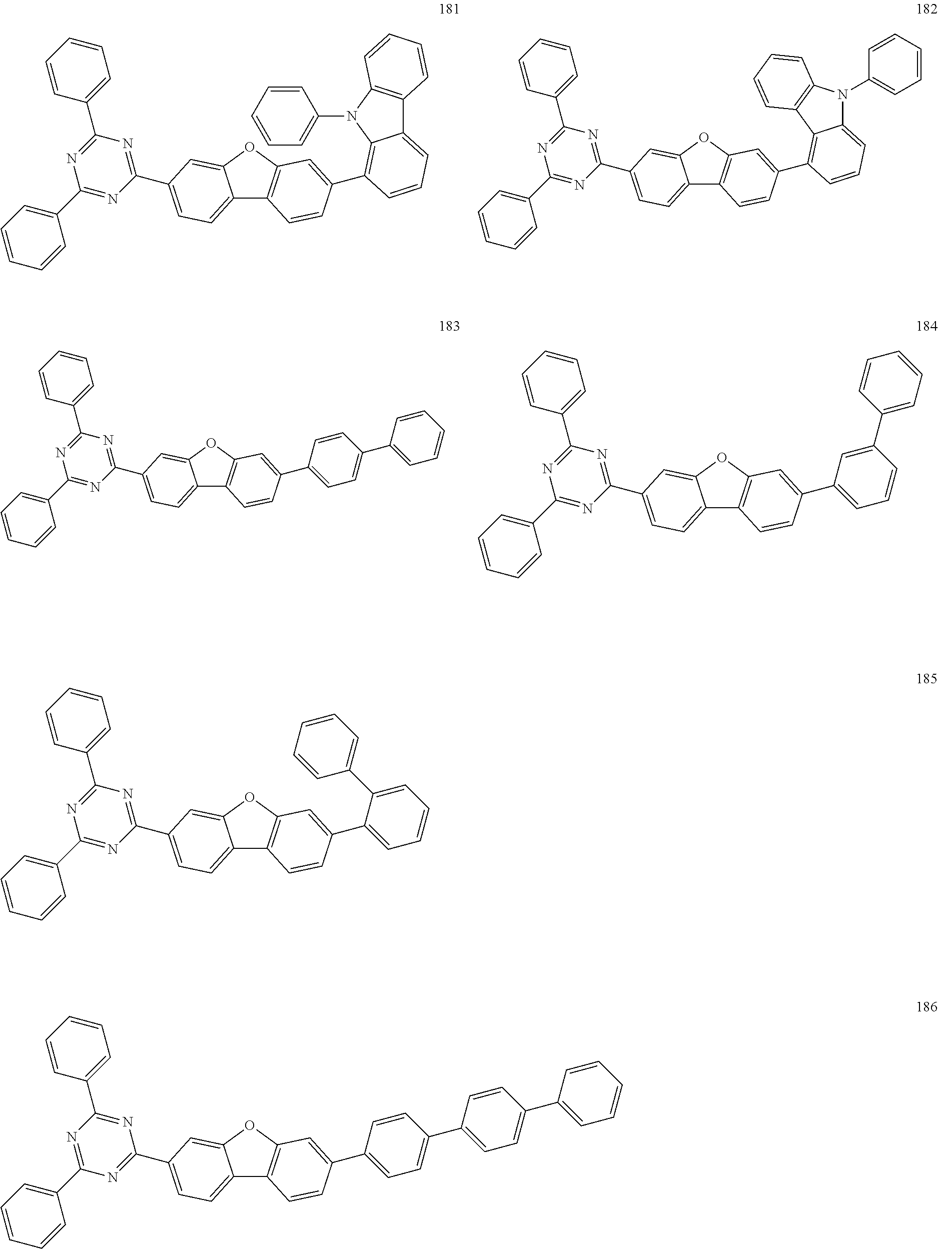 [SMILES: c1ccc(-c2nc(-c3ccccc3)nc(-c3ccc4c(c3)oc3cc(-c5cccc6c7ccccc7n(-c7ccccc7)c56)ccc34)n2)cc1 ; c1ccc(-c2nc(-c3ccccc3)nc(-c3ccc4c(c3)oc3cc(-c5cccc6c5c5ccccc5n6-c5ccccc5)ccc34)n2)cc1 ; c1ccc(-c2ccc(-c3ccc4c(c3)oc3cc(-c5nc(-c6ccccc6)nc(-c6ccccc6)n5)ccc34)cc2)cc1 ; c1ccc(-c2cccc(-c3ccc4c(c3)oc3cc(-c5nc(-c6ccccc6)nc(-c6ccccc6)n5)ccc34)c2)cc1 ; c1ccc(-c2nc(-c3ccccc3)nc(-c3ccc4c(c3)oc3cc(-c5ccccc5-c5ccccc5)ccc34)n2)cc1 ; c1ccc(-c2ccc(-c3ccc(-c4ccc5c(c4)oc4cc(-c6nc(-c7ccccc7)nc(-c7ccccc7)n6)ccc45)cc3)cc2)cc1]

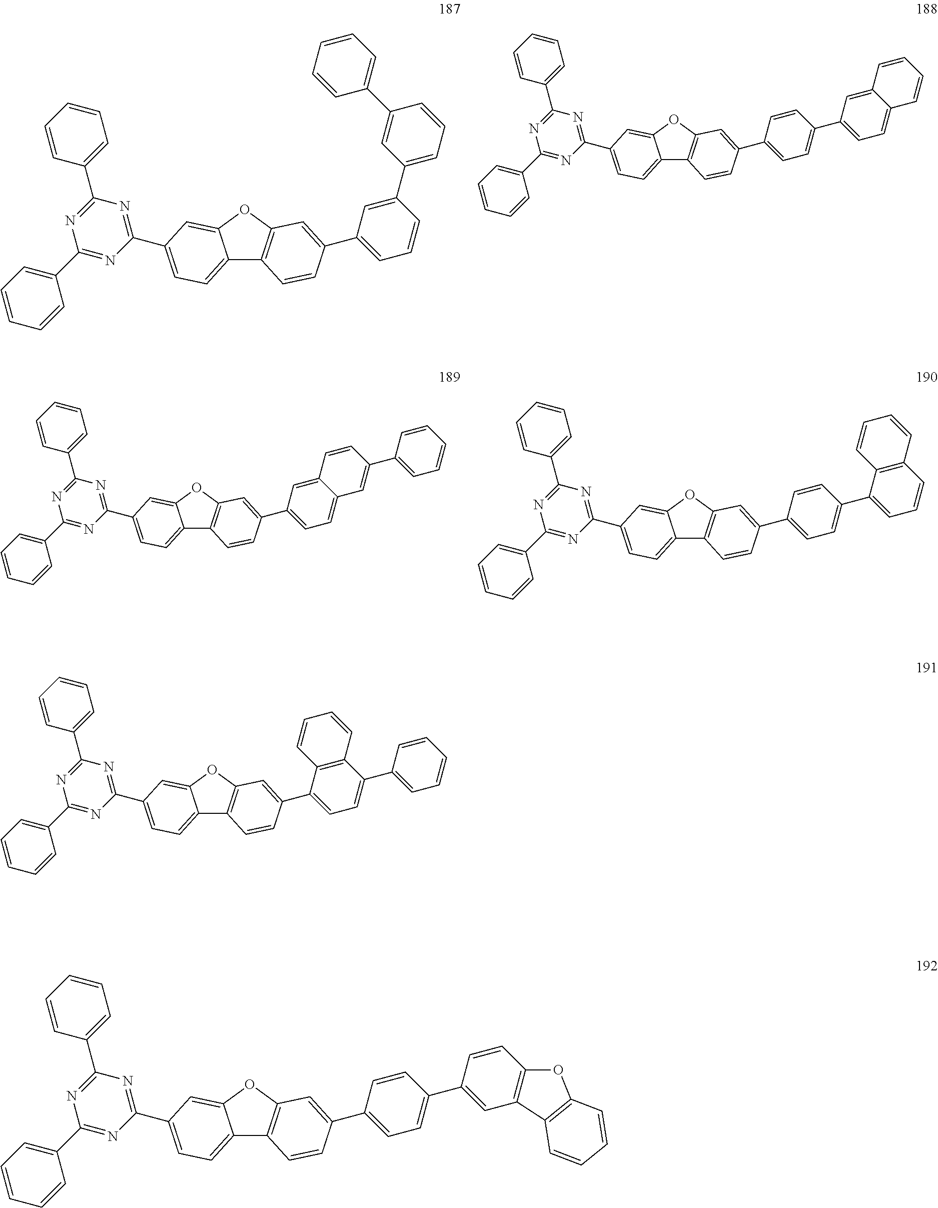

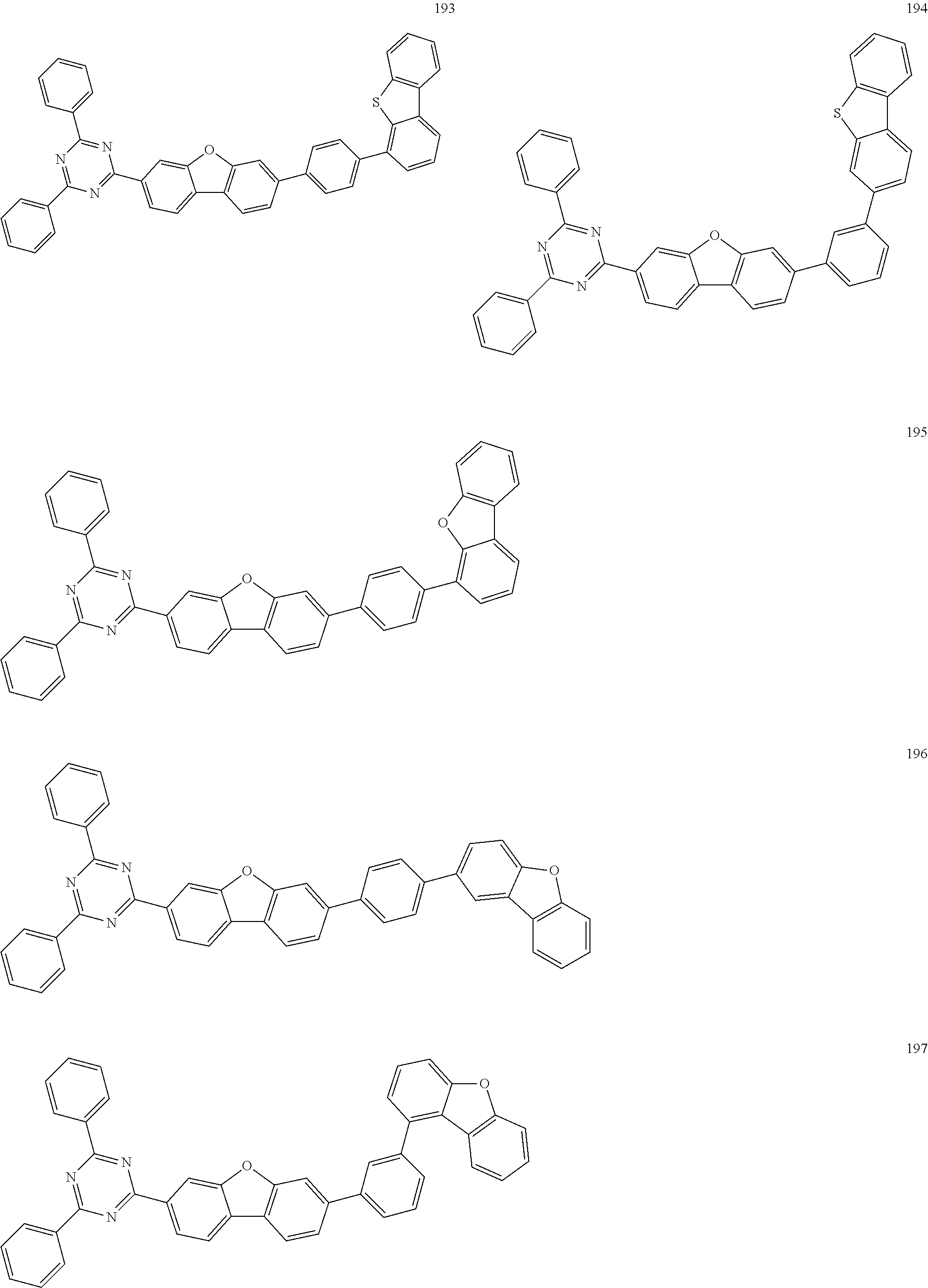 [SMILES: c1ccc(-c2nc(-c3ccccc3)nc(-c3ccc4c(c3)oc3cc(-c5ccc(-c6cccc7c6sc6ccccc67)cc5)ccc34)n2)cc1 ; c1ccc(-c2nc(-c3ccccc3)nc(-c3ccc4c(c3)oc3cc(-c5cccc(-c6ccc7c(c6)sc6ccccc67)c5)ccc34)n2)cc1 ; c1ccc(-c2nc(-c3ccccc3)nc(-c3ccc4c(c3)oc3cc(-c5ccc(-c6cccc7c6oc6ccccc67)cc5)ccc34)n2)cc1 ; c1ccc(-c2nc(-c3ccccc3)nc(-c3ccc4c(c3)oc3cc(-c5ccc(-c6ccc7oc8ccccc8c7c6)cc5)ccc34)n2)cc1 ; c1ccc(-c2nc(-c3ccccc3)nc(-c3ccc4c(c3)oc3cc(-c5cccc(-c6cccc7oc8ccccc8c67)c5)ccc34)n2)cc1]

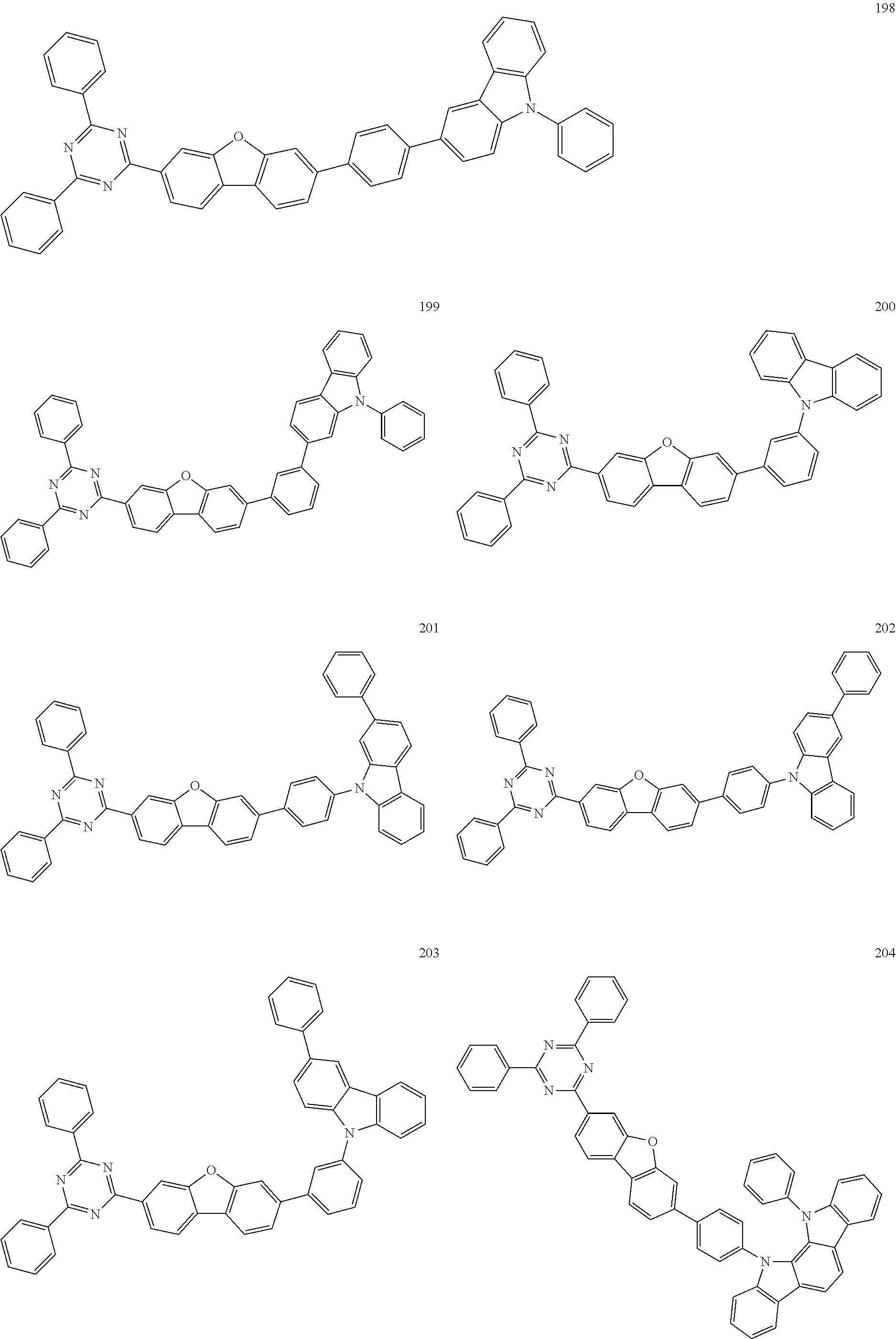

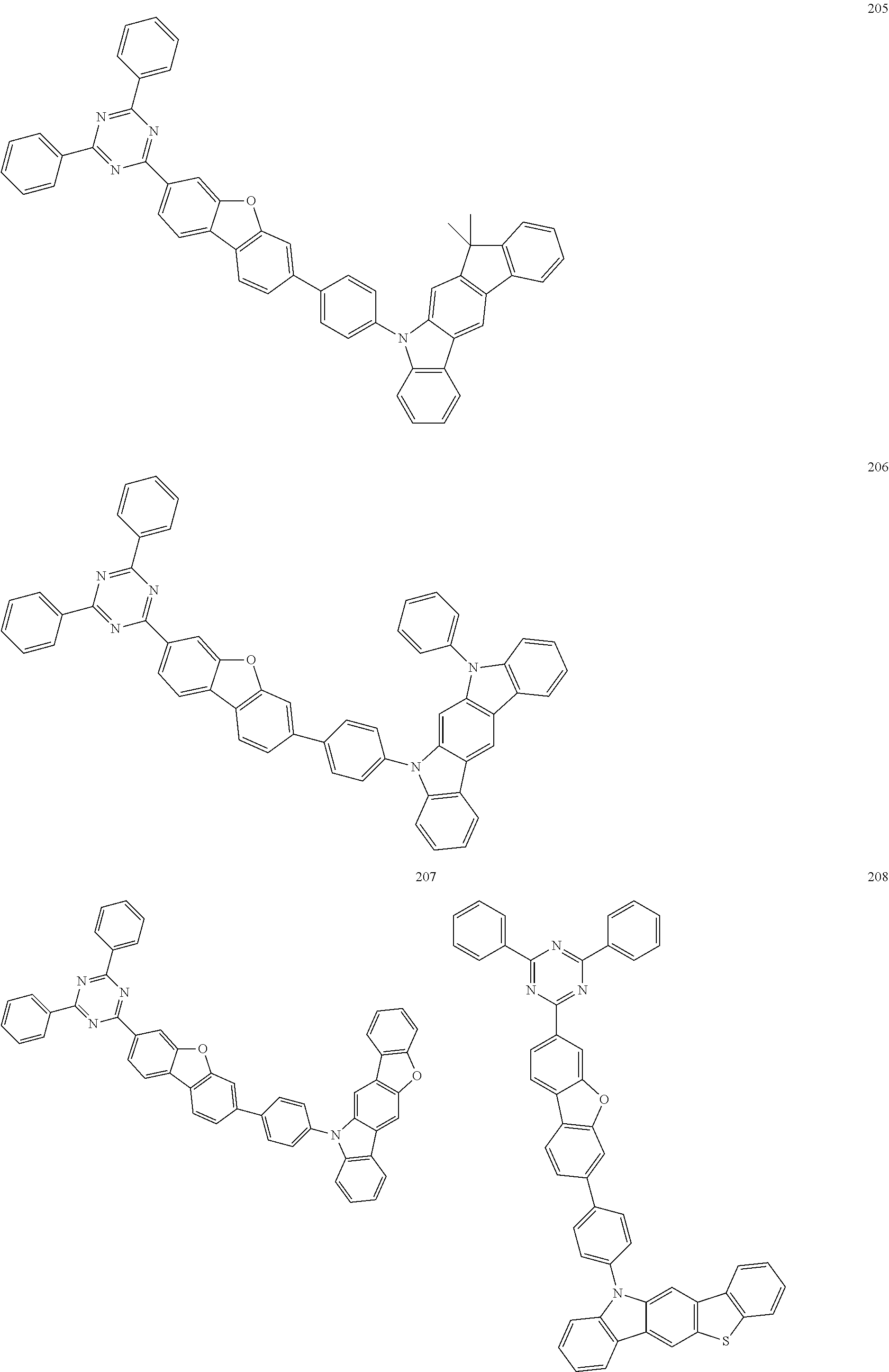 [SMILES: CC1(C)c2ccccc2-c2cc3c4ccccc4n(-c4ccc(-c5ccc6c(c5)oc5cc(-c7nc(-c8ccccc8)nc(-c8ccccc8)n7)ccc56)cc4)c3cc21 ; c1ccc(-c2nc(-c3ccccc3)nc(-c3ccc4c(c3)oc3cc(-c5ccc(-n6c7ccccc7c7cc8c9ccccc9n(-c9ccccc9)c8cc76)cc5)ccc34)n2)cc1 ; c1ccc(-c2nc(-c3ccccc3)nc(-c3ccc4c(c3)oc3cc(-c5ccc(-n6c7ccccc7c7cc8oc9ccccc9c8cc76)cc5)ccc34)n2)cc1 ; c1ccc(-c2nc(-c3ccccc3)nc(-c3ccc4c(c3)oc3cc(-c5ccc(-n6c7ccccc7c7cc8sc9ccccc9c8cc76)cc5)ccc34)n2)cc1]

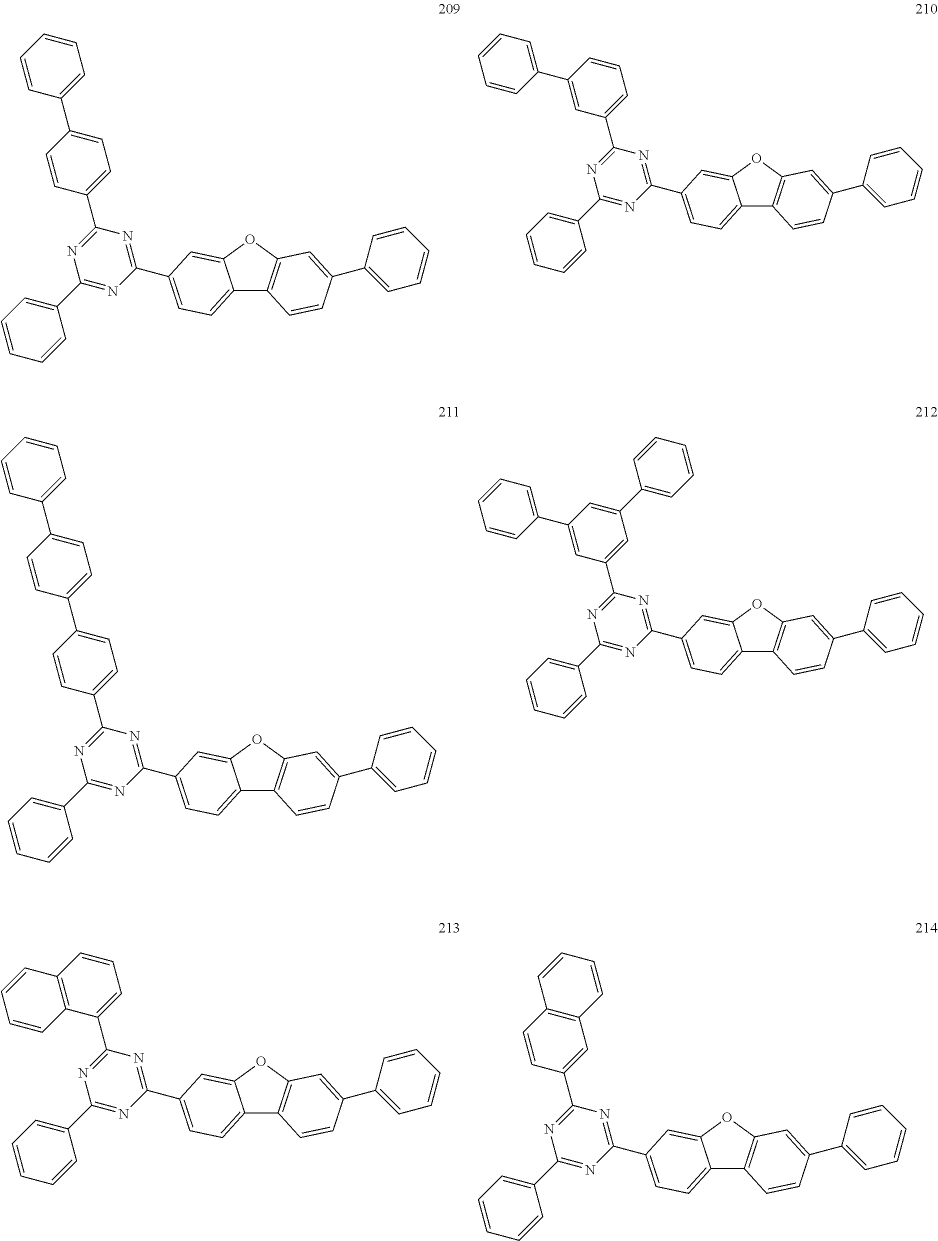 [SMILES: c1ccc(-c2ccc(-c3nc(-c4ccccc4)nc(-c4ccc5c(c4)oc4cc(-c6ccccc6)ccc45)n3)cc2)cc1 ; c1ccc(-c2cccc(-c3nc(-c4ccccc4)nc(-c4ccc5c(c4)oc4cc(-c6ccccc6)ccc45)n3)c2)cc1 ; c1ccc(-c2ccc(-c3ccc(-c4nc(-c5ccccc5)nc(-c5ccc6c(c5)oc5cc(-c7ccccc7)ccc56)n4)cc3)cc2)cc1 ; c1ccc(-c2cc(-c3ccccc3)cc(-c3nc(-c4ccccc4)nc(-c4ccc5c(c4)oc4cc(-c6ccccc6)ccc45)n3)c2)cc1 ; c1ccc(-c2ccc3c(c2)oc2cc(-c4nc(-c5ccccc5)nc(-c5cccc6ccccc56)n4)ccc23)cc1 ; c1ccc(-c2ccc3c(c2)oc2cc(-c4nc(-c5ccccc5)nc(-c5ccc6ccccc6c5)n4)ccc23)cc1]

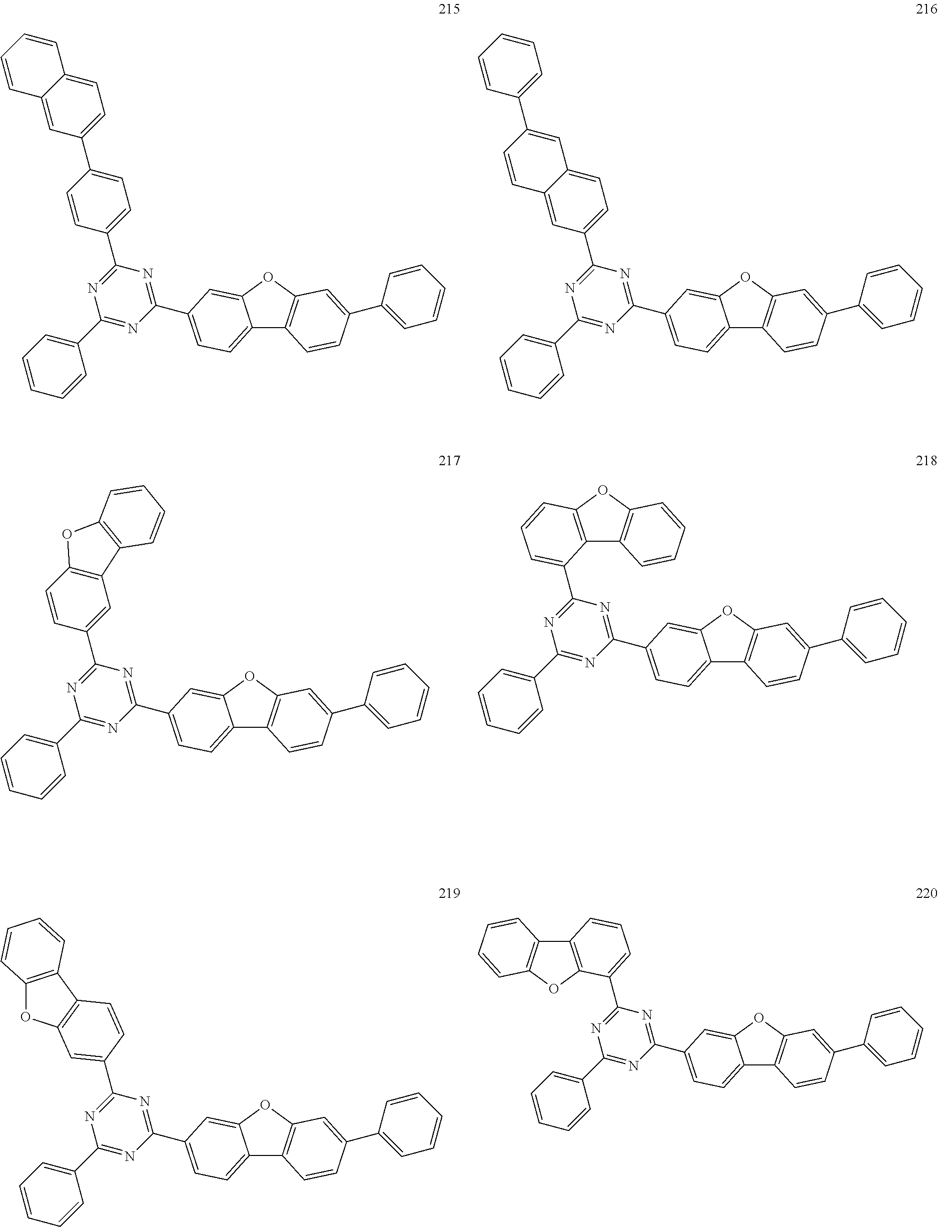 [SMILES: c1ccc(-c2ccc3c(c2)oc2cc(-c4nc(-c5ccccc5)nc(-c5ccc(-c6ccc7ccccc7c6)cc5)n4)ccc23)cc1 ; c1ccc(-c2ccc3cc(-c4nc(-c5ccccc5)nc(-c5ccc6c(c5)oc5cc(-c7ccccc7)ccc56)n4)ccc3c2)cc1 ; c1ccc(-c2ccc3c(c2)oc2cc(-c4nc(-c5ccccc5)nc(-c5ccc6oc7ccccc7c6c5)n4)ccc23)cc1 ; c1ccc(-c2ccc3c(c2)oc2cc(-c4nc(-c5ccccc5)nc(-c5cccc6oc7ccccc7c56)n4)ccc23)cc1 ; c1ccc(-c2ccc3c(c2)oc2cc(-c4nc(-c5ccccc5)nc(-c5ccc6c(c5)oc5ccccc56)n4)ccc23)cc1 ; c1ccc(-c2ccc3c(c2)oc2cc(-c4nc(-c5ccccc5)nc(-c5cccc6c5oc5ccccc56)n4)ccc23)cc1]

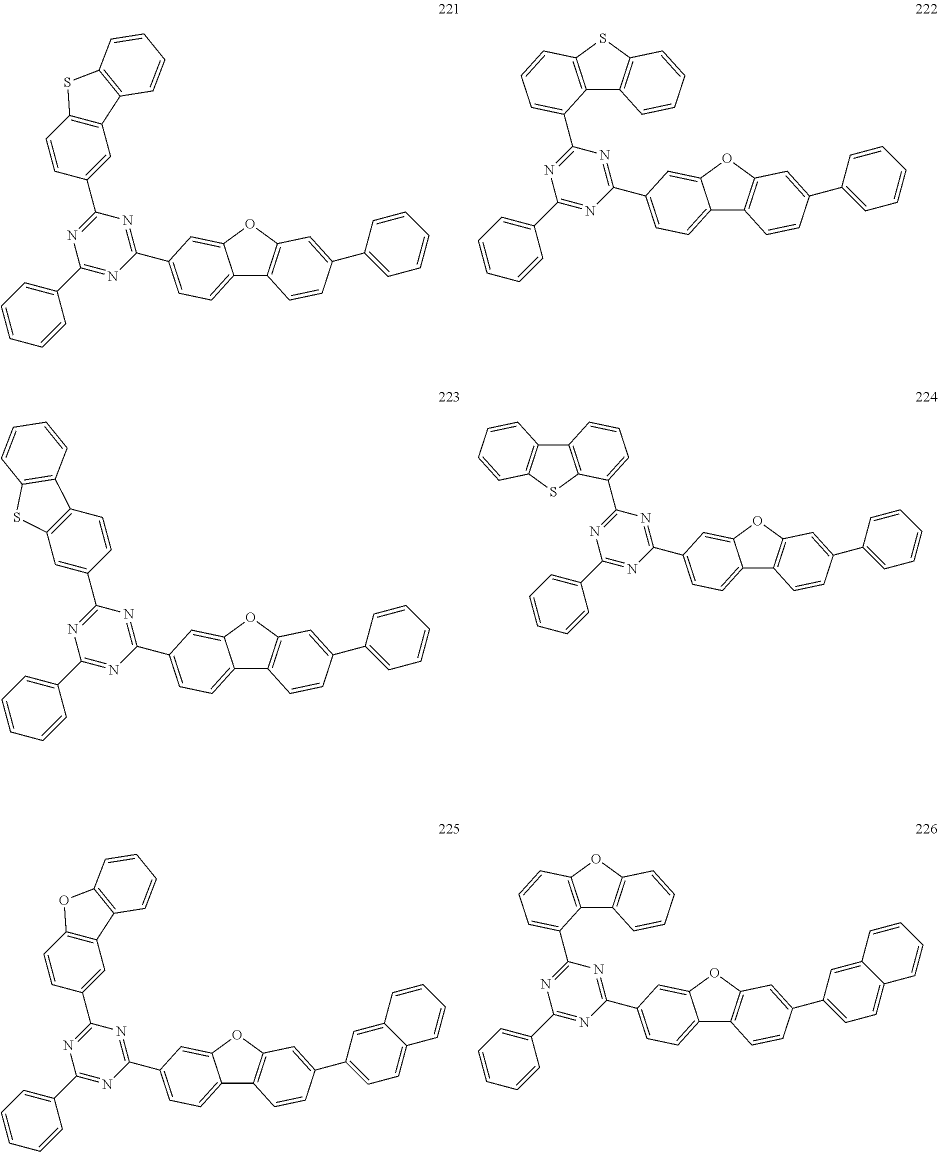

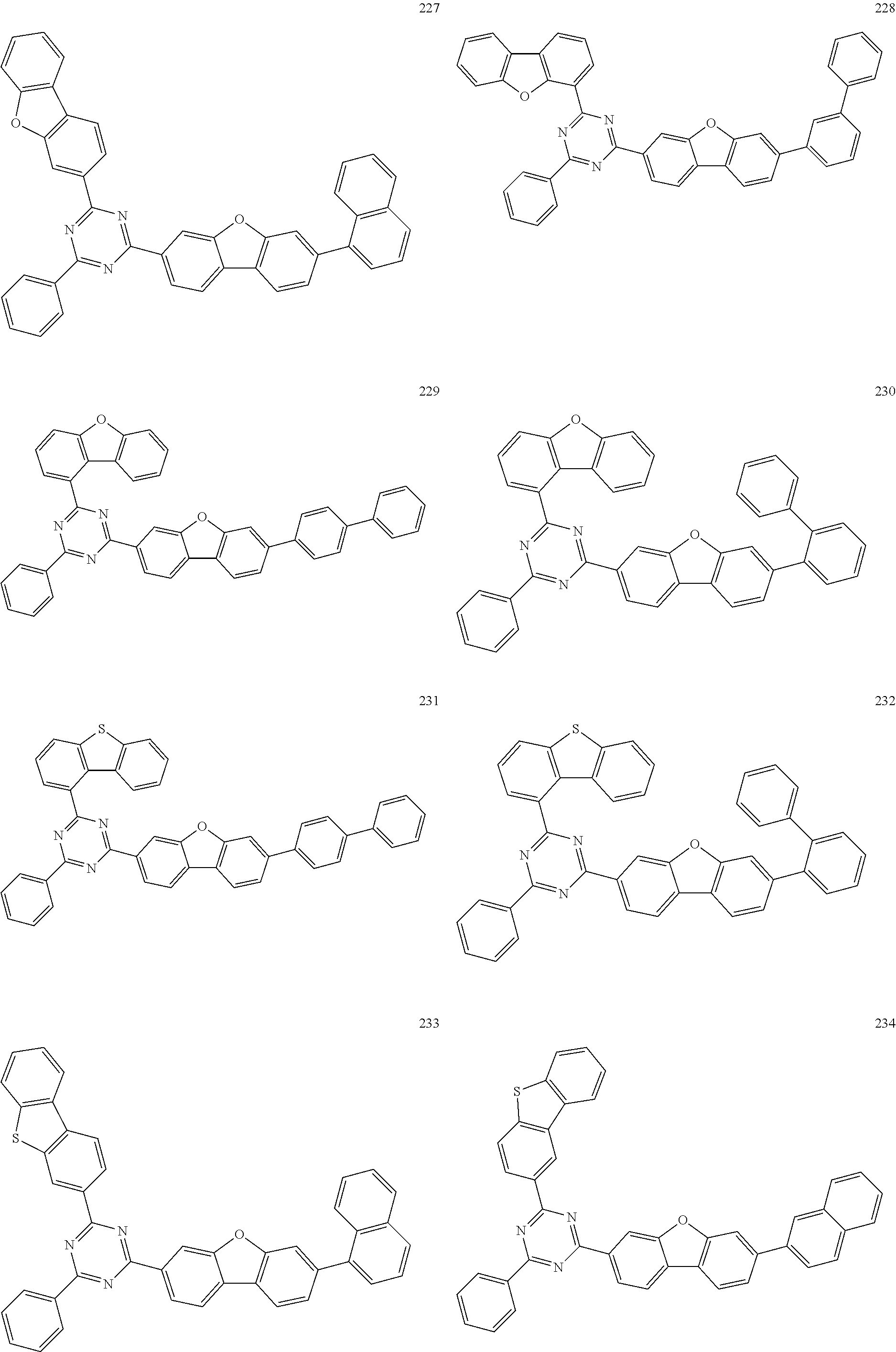

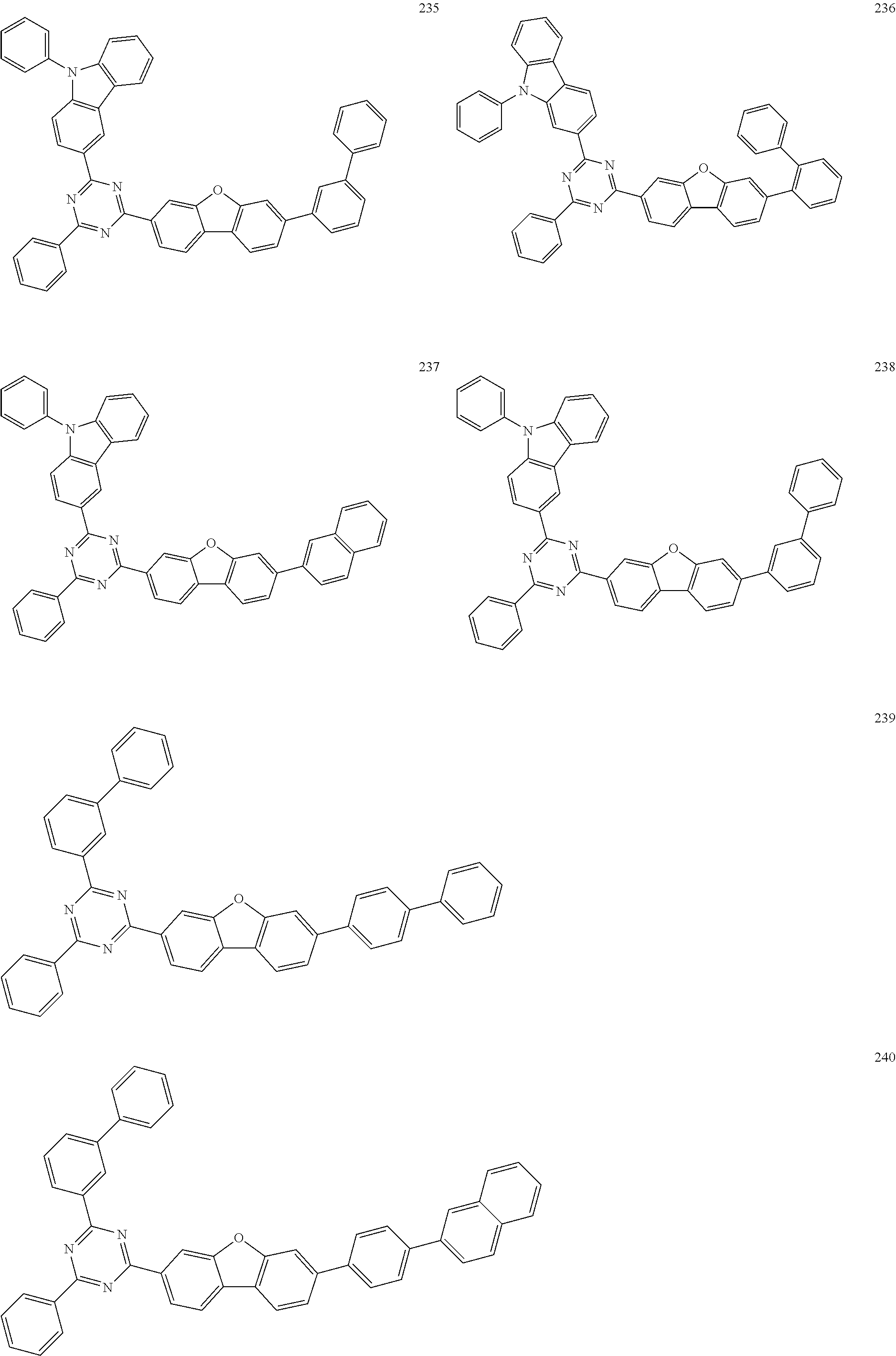

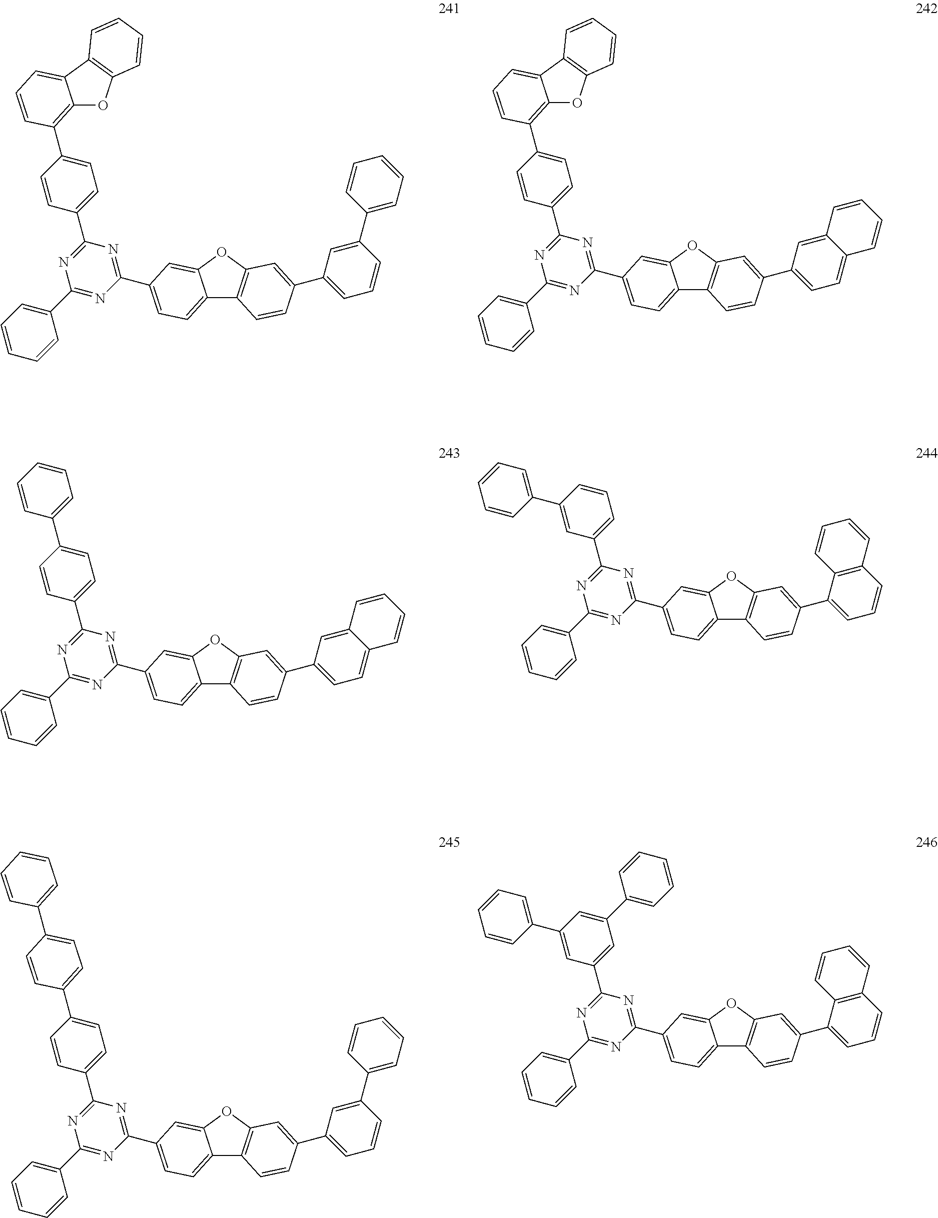

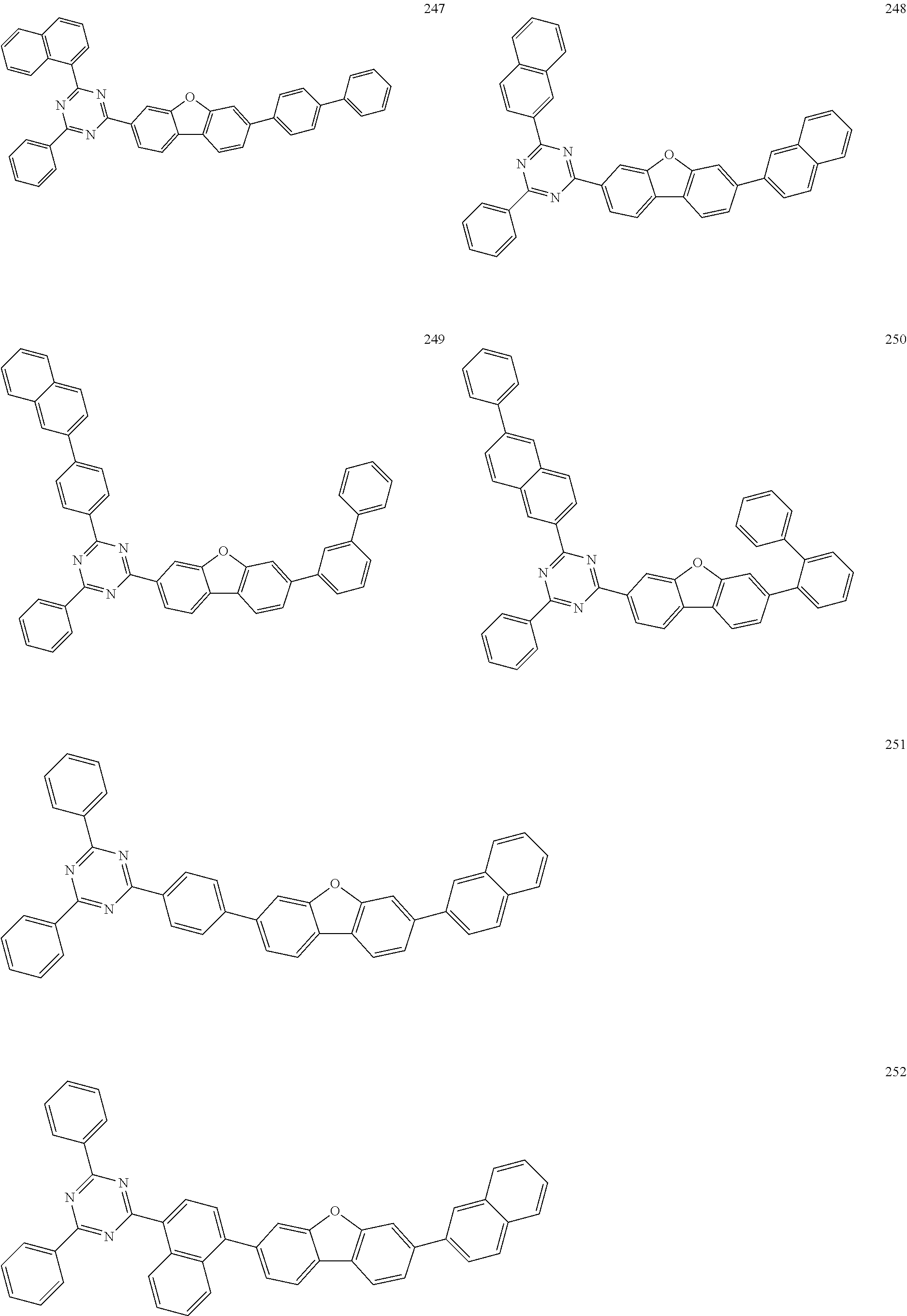

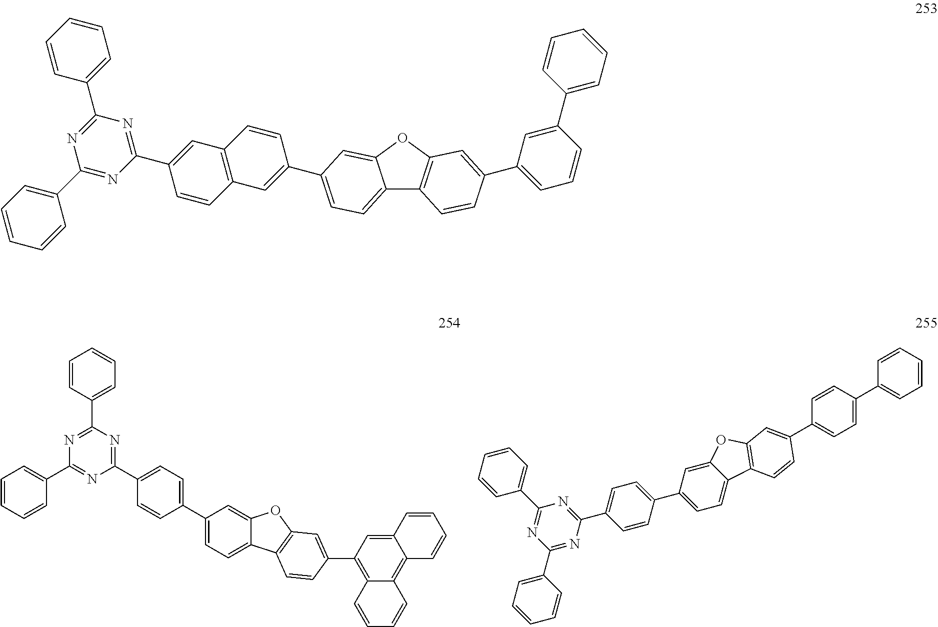 [SMILES: c1ccc(-c2cccc(-c3ccc4c(c3)oc3cc(-c5ccc6cc(-c7nc(-c8ccccc8)nc(-c8ccccc8)n7)ccc6c5)ccc34)c2)cc1 ; c1ccc(-c2nc(-c3ccccc3)nc(-c3ccc(-c4ccc5c(c4)oc4cc(-c6cc7ccccc7c7ccccc67)ccc45)cc3)n2)cc1 ; c1ccc(-c2ccc(-c3ccc4c(c3)oc3cc(-c5ccc(-c6nc(-c7ccccc7)nc(-c7ccccc7)n6)cc5)ccc34)cc2)cc1]

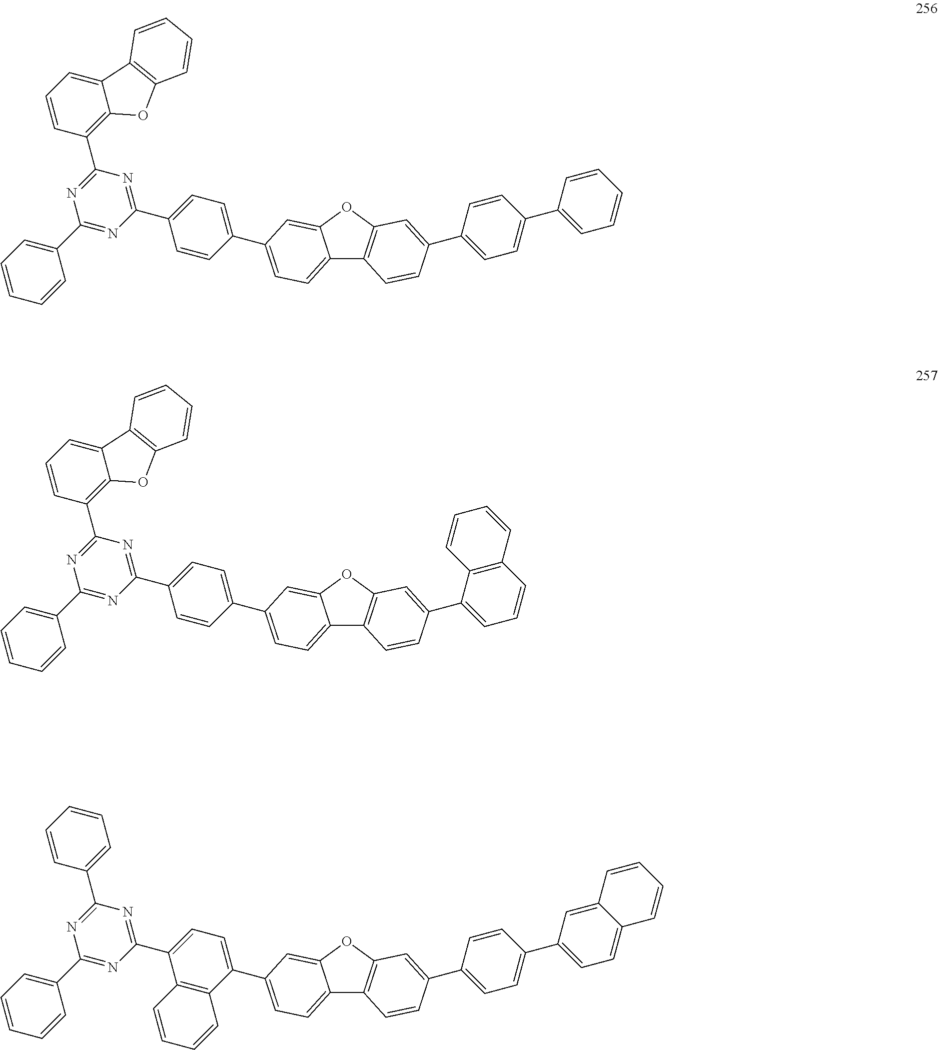 [SMILES: c1ccc(-c2ccc(-c3ccc4c(c3)oc3cc(-c5ccc(-c6nc(-c7ccccc7)nc(-c7cccc8c7oc7ccccc78)n6)cc5)ccc34)cc2)cc1 ; c1ccc(-c2nc(-c3ccc(-c4ccc5c(c4)oc4cc(-c6cccc7ccccc67)ccc45)cc3)nc(-c3cccc4c3oc3ccccc34)n2)cc1 ; c1ccc(-c2nc(-c3ccccc3)nc(-c3ccc(-c4ccc5c(c4)oc4cc(-c6ccc(-c7ccc8ccccc8c7)cc6)ccc45)c4ccccc34)n2)cc1]

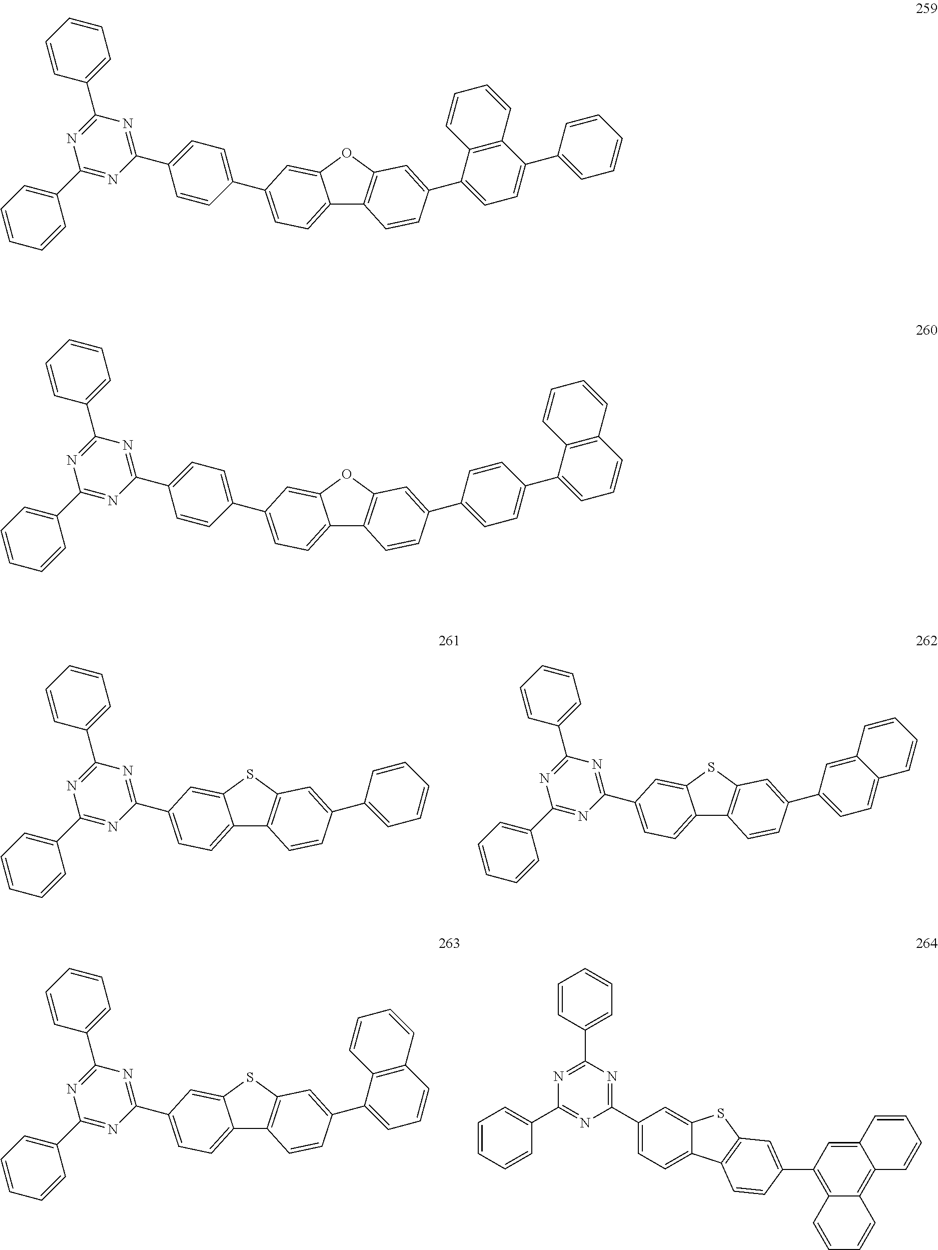 [SMILES: c1ccc(-c2nc(-c3ccccc3)nc(-c3ccc(-c4ccc5c(c4)oc4cc(-c6ccc(-c7ccccc7)c7ccccc67)ccc45)cc3)n2)cc1 ; c1ccc(-c2nc(-c3ccccc3)nc(-c3ccc(-c4ccc5c(c4)oc4cc(-c6ccc(-c7cccc8ccccc78)cc6)ccc45)cc3)n2)cc1 ; c1ccc(-c2ccc3c(c2)sc2cc(-c4nc(-c5ccccc5)nc(-c5ccccc5)n4)ccc23)cc1 ; c1ccc(-c2nc(-c3ccccc3)nc(-c3ccc4c(c3)sc3cc(-c5ccc6ccccc6c5)ccc34)n2)cc1 ; c1ccc(-c2nc(-c3ccccc3)nc(-c3ccc4c(c3)sc3cc(-c5cccc6ccccc56)ccc34)n2)cc1 ; c1ccc(-c2nc(-c3ccccc3)nc(-c3ccc4c(c3)sc3cc(-c5cc6ccccc6c6ccccc56)ccc34)n2)cc1]

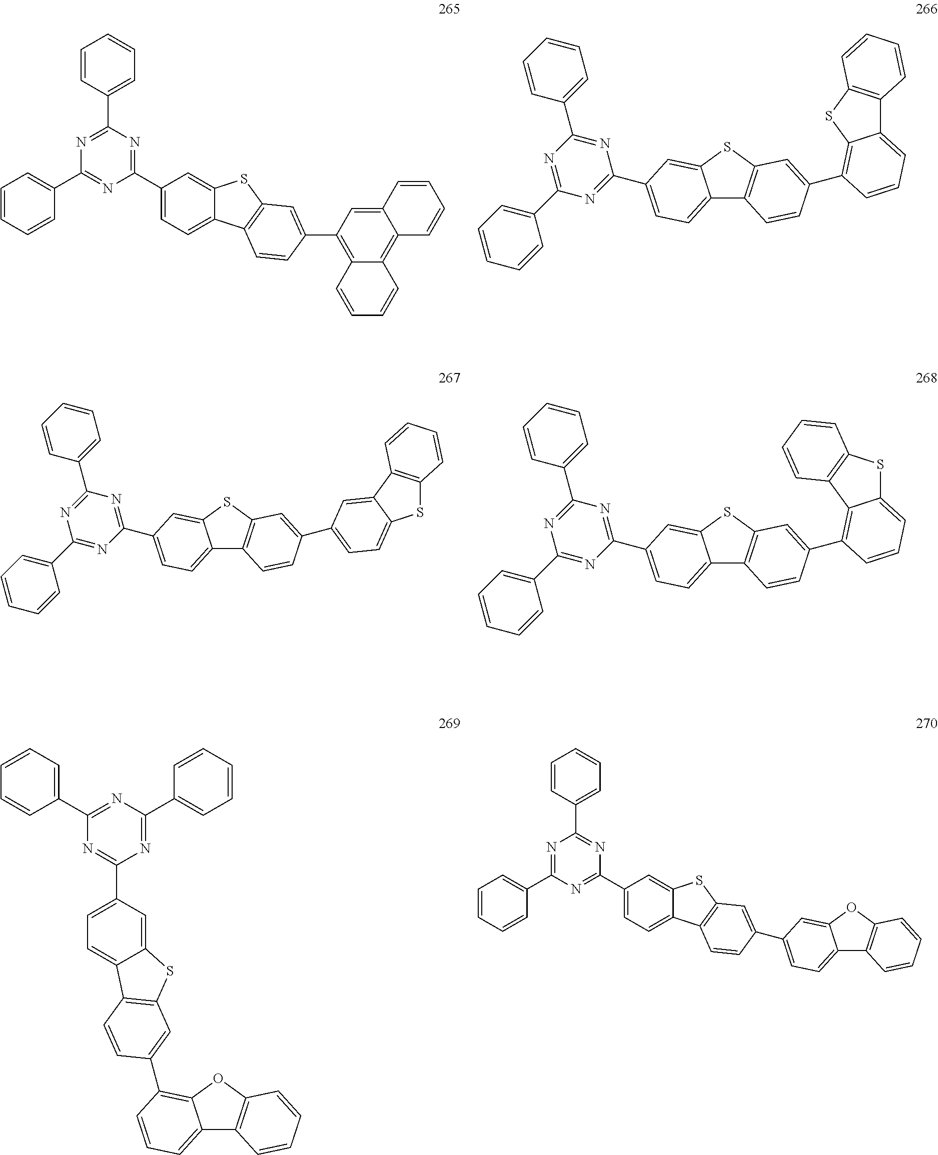

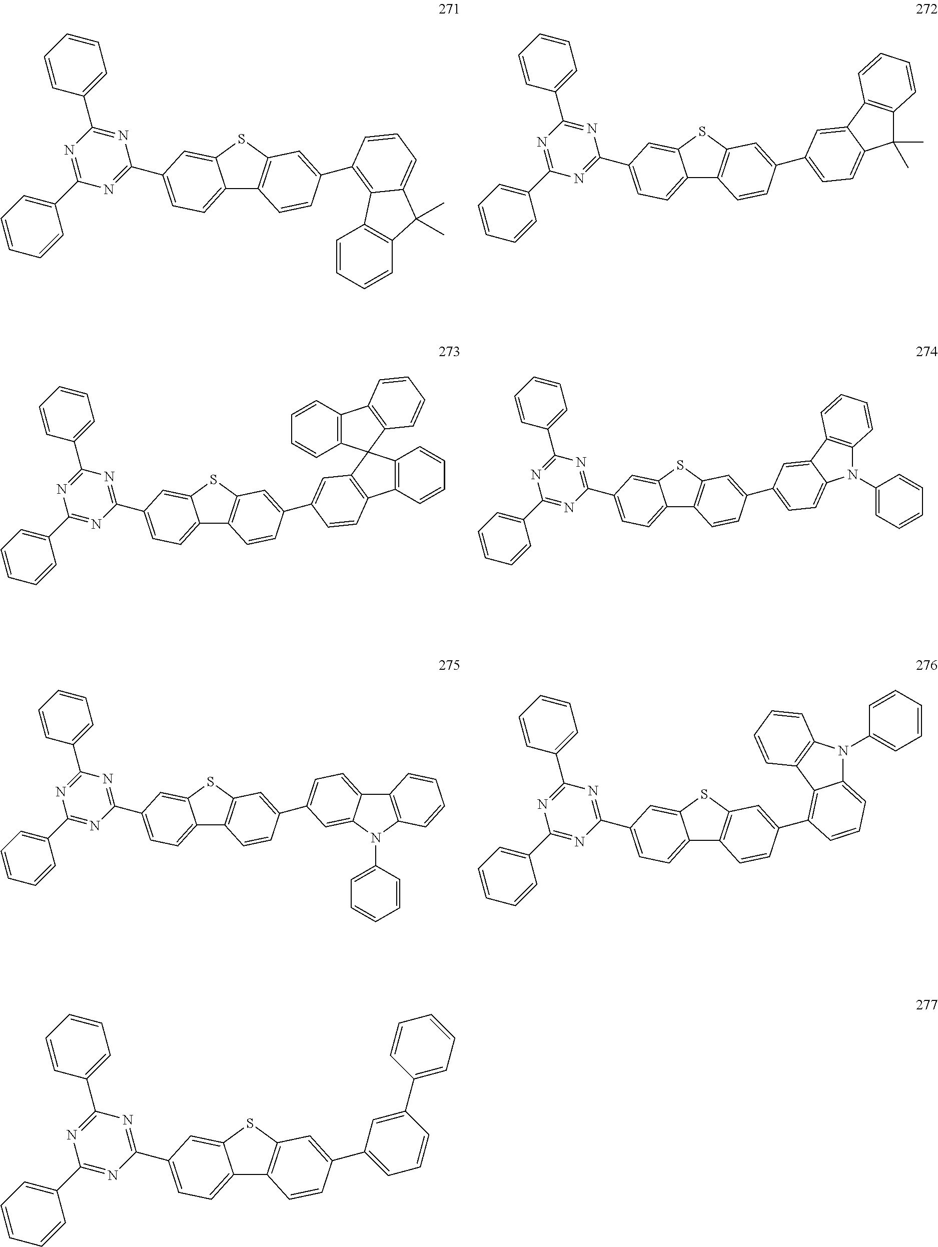

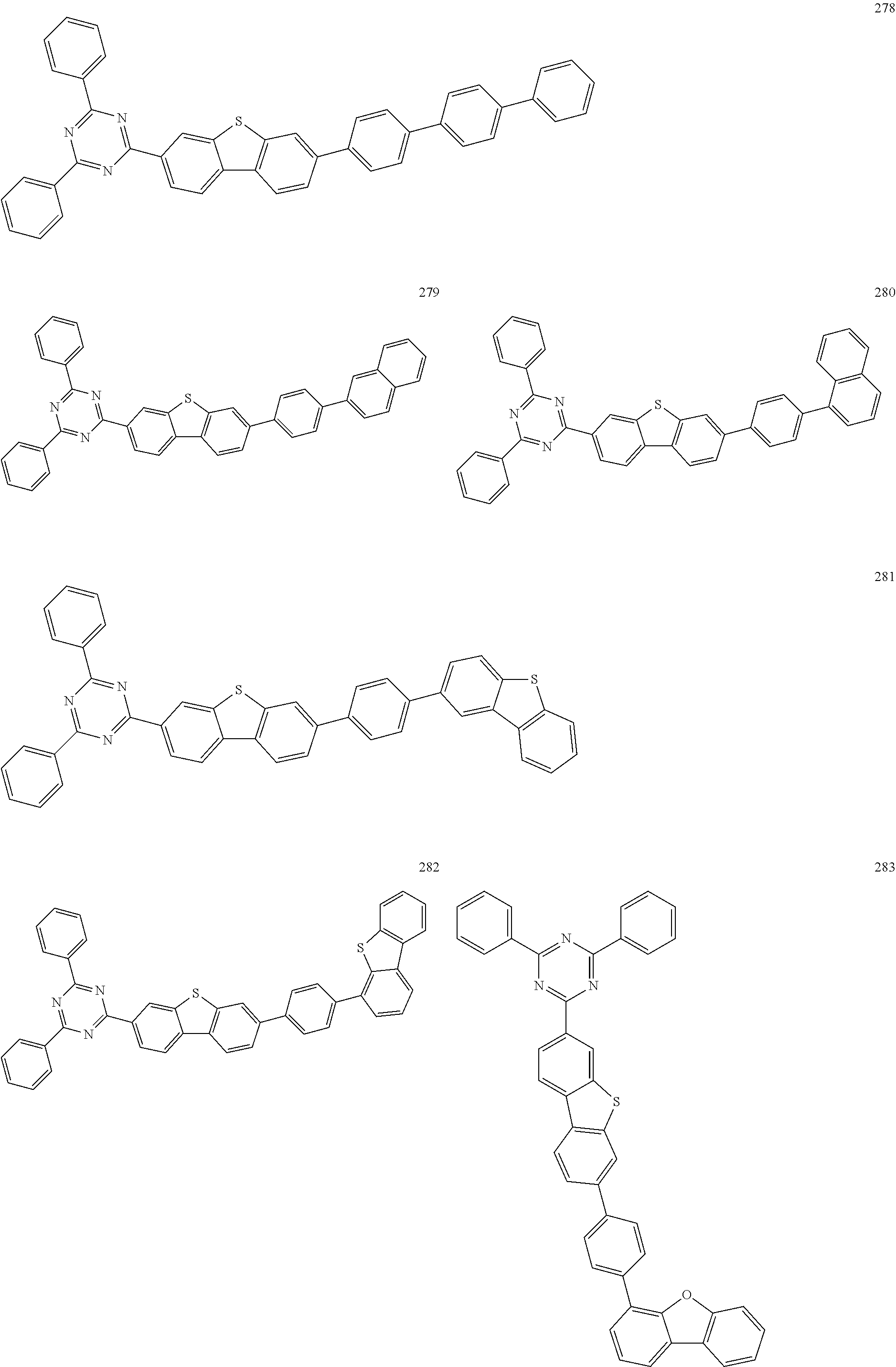

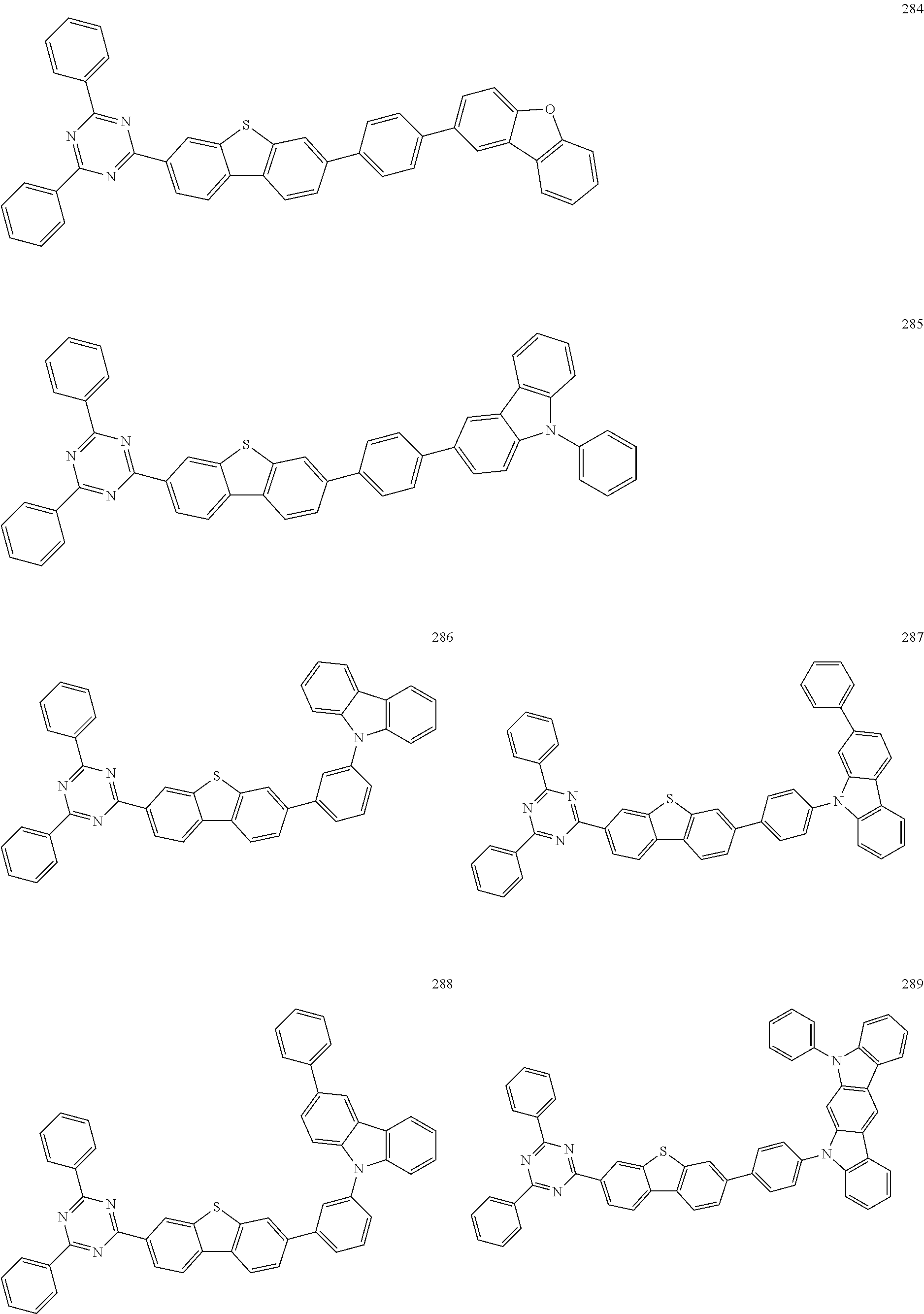

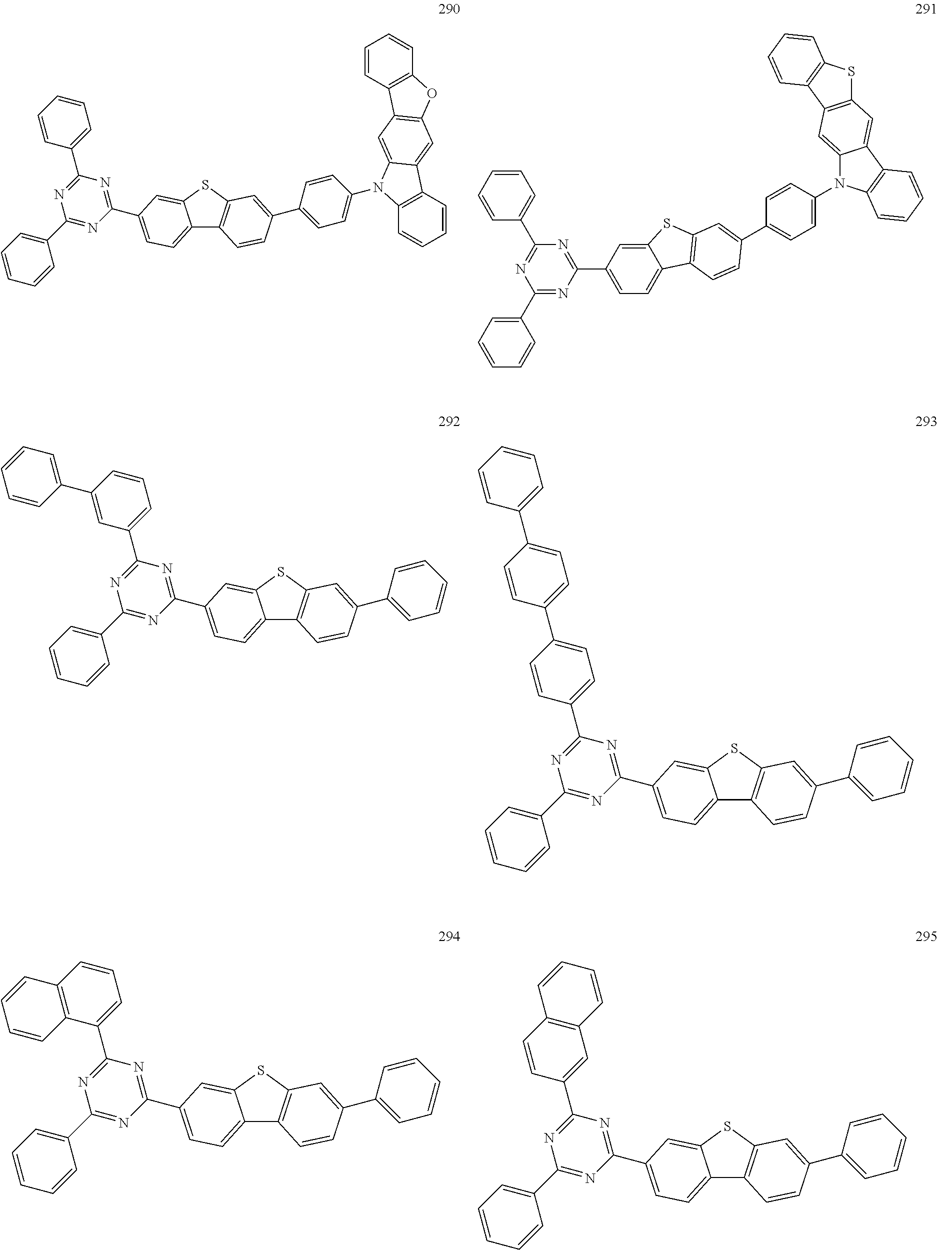

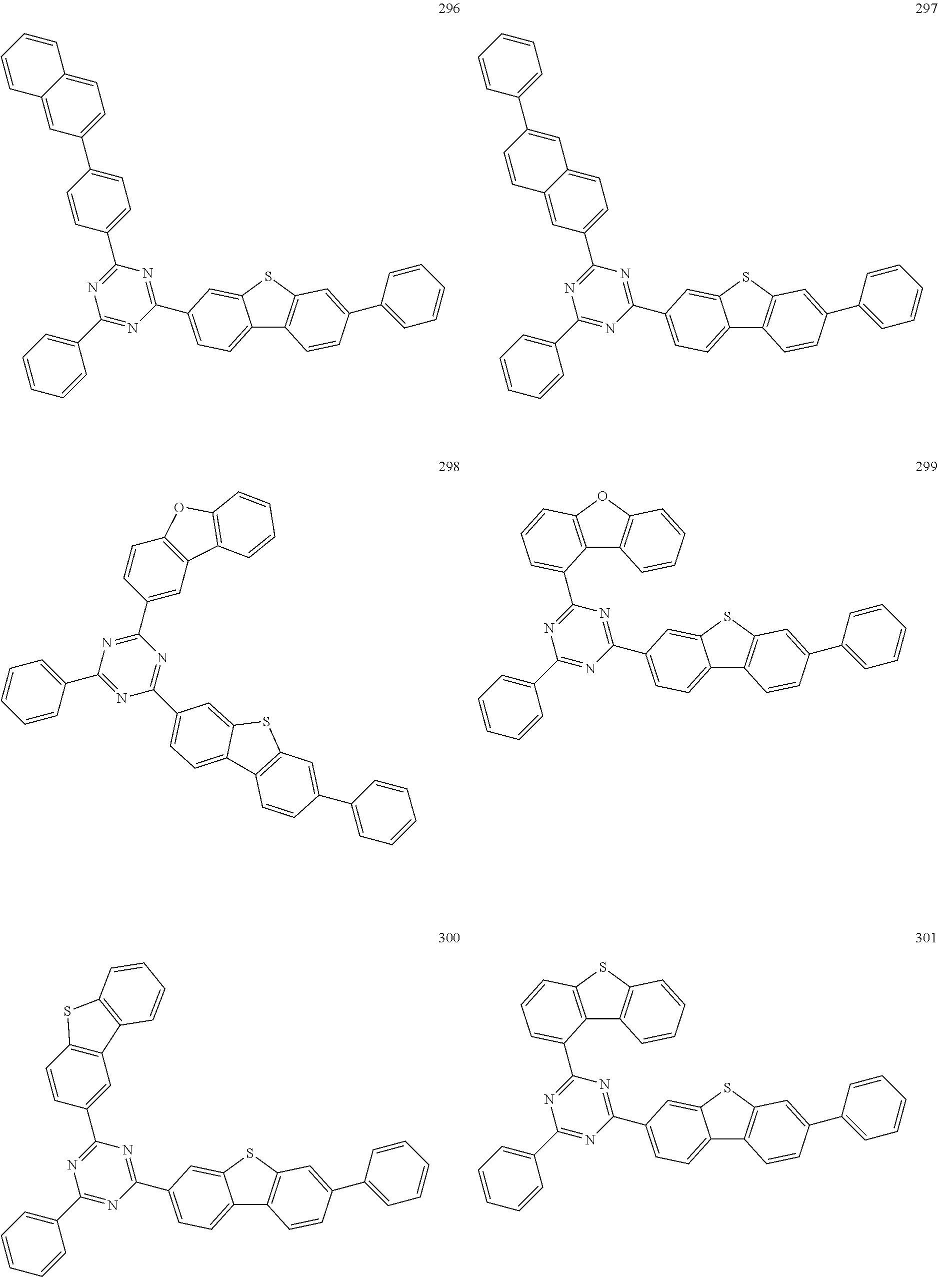 [SMILES: c1ccc(-c2ccc3c(c2)sc2cc(-c4nc(-c5ccccc5)nc(-c5ccc(-c6ccc7ccccc7c6)cc5)n4)ccc23)cc1 ; c1ccc(-c2ccc3cc(-c4nc(-c5ccccc5)nc(-c5ccc6c(c5)sc5cc(-c7ccccc7)ccc56)n4)ccc3c2)cc1 ; c1ccc(-c2ccc3c(c2)sc2cc(-c4nc(-c5ccccc5)nc(-c5ccc6oc7ccccc7c6c5)n4)ccc23)cc1 ; c1ccc(-c2ccc3c(c2)sc2cc(-c4nc(-c5ccccc5)nc(-c5cccc6oc7ccccc7c56)n4)ccc23)cc1 ; c1ccc(-c2ccc3c(c2)sc2cc(-c4nc(-c5ccccc5)nc(-c5ccc6sc7ccccc7c6c5)n4)ccc23)cc1 ; c1ccc(-c2ccc3c(c2)sc2cc(-c4nc(-c5ccccc5)nc(-c5cccc6sc7ccccc7c56)n4)ccc23)cc1]

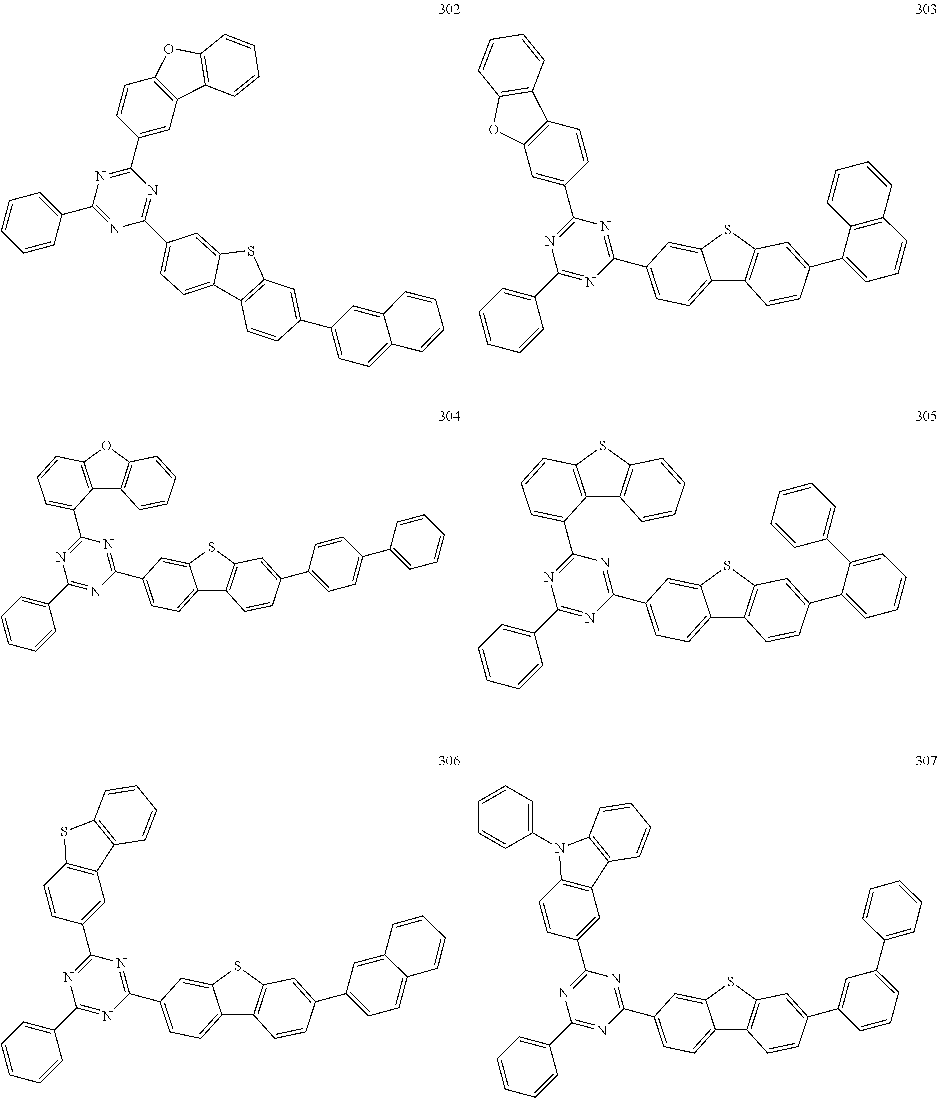

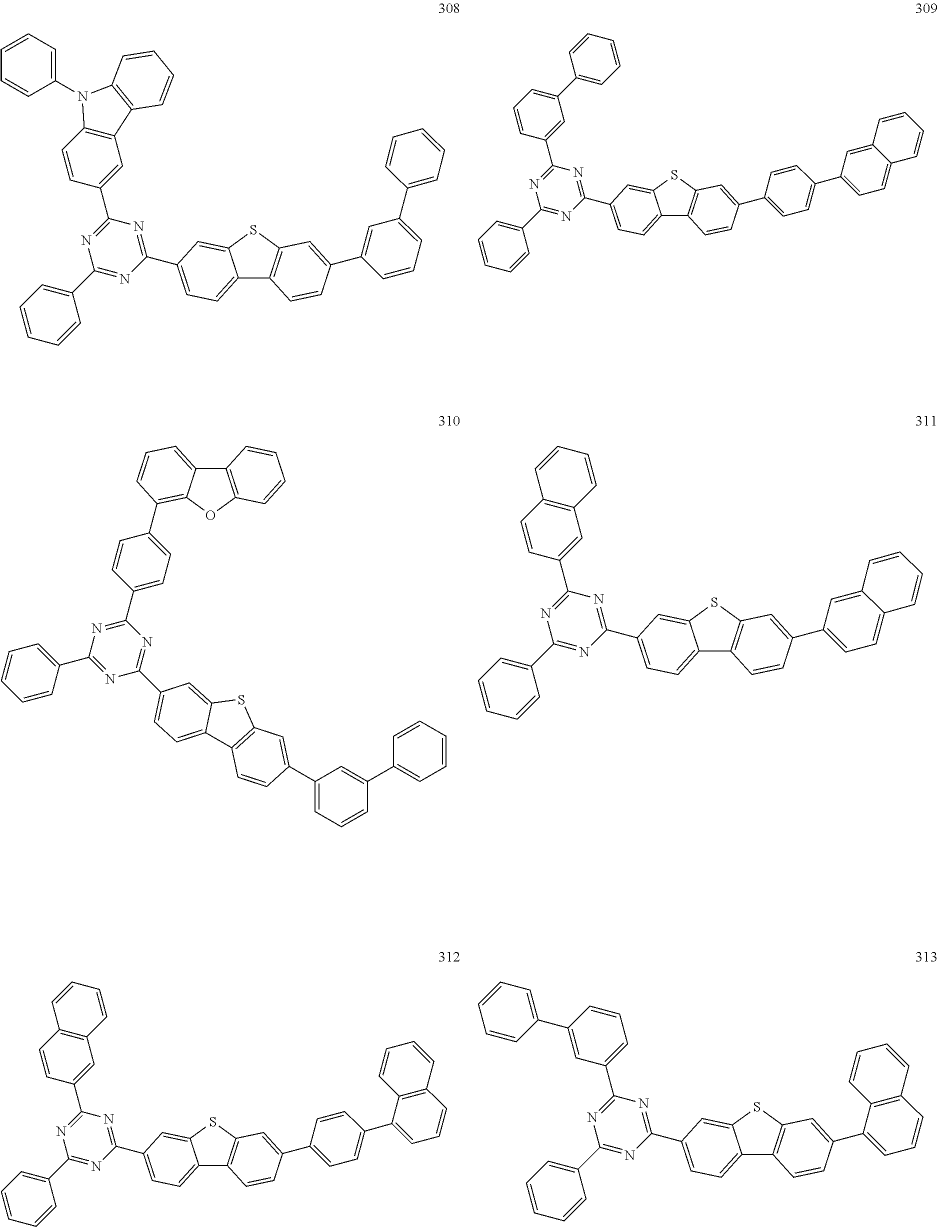

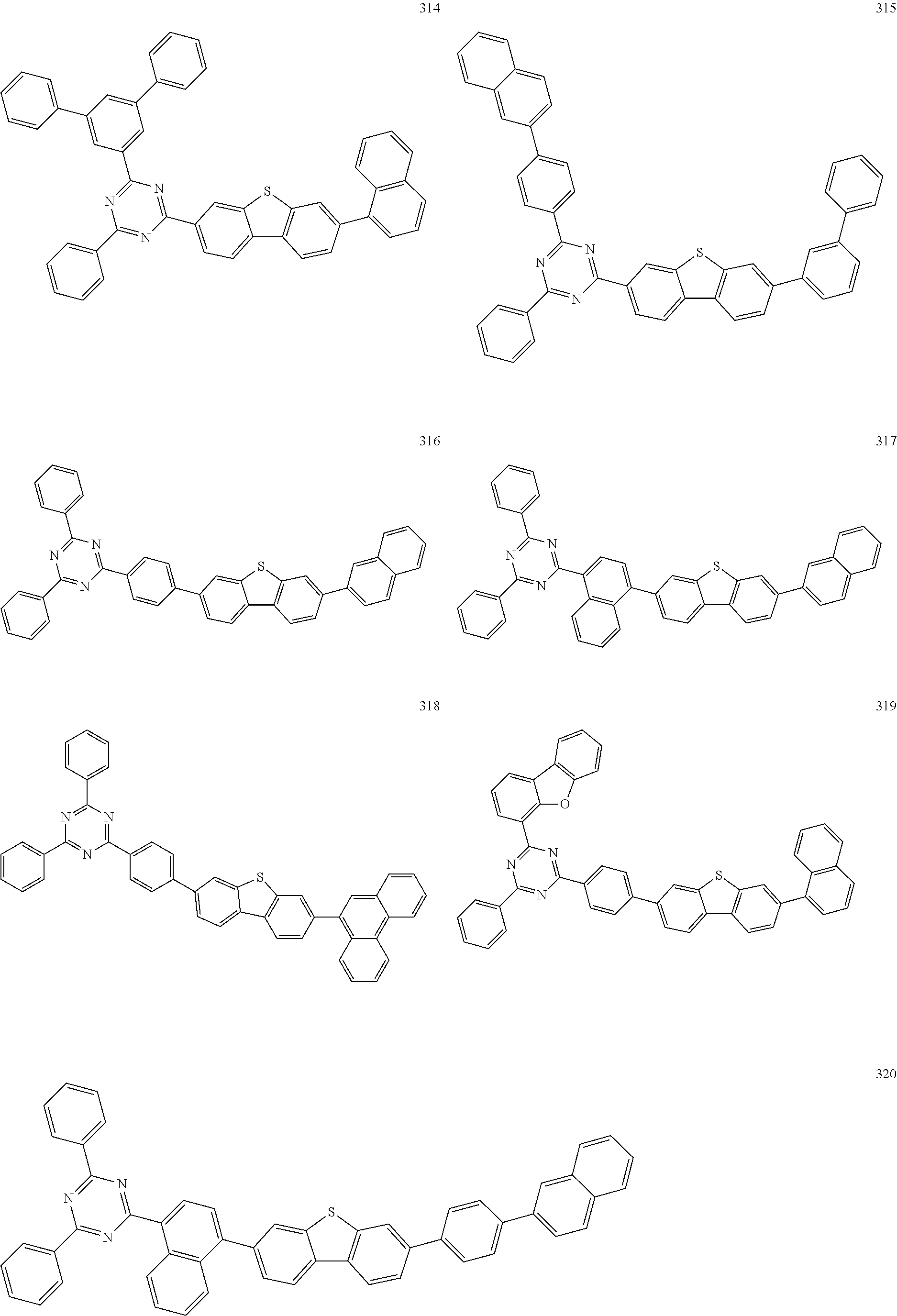

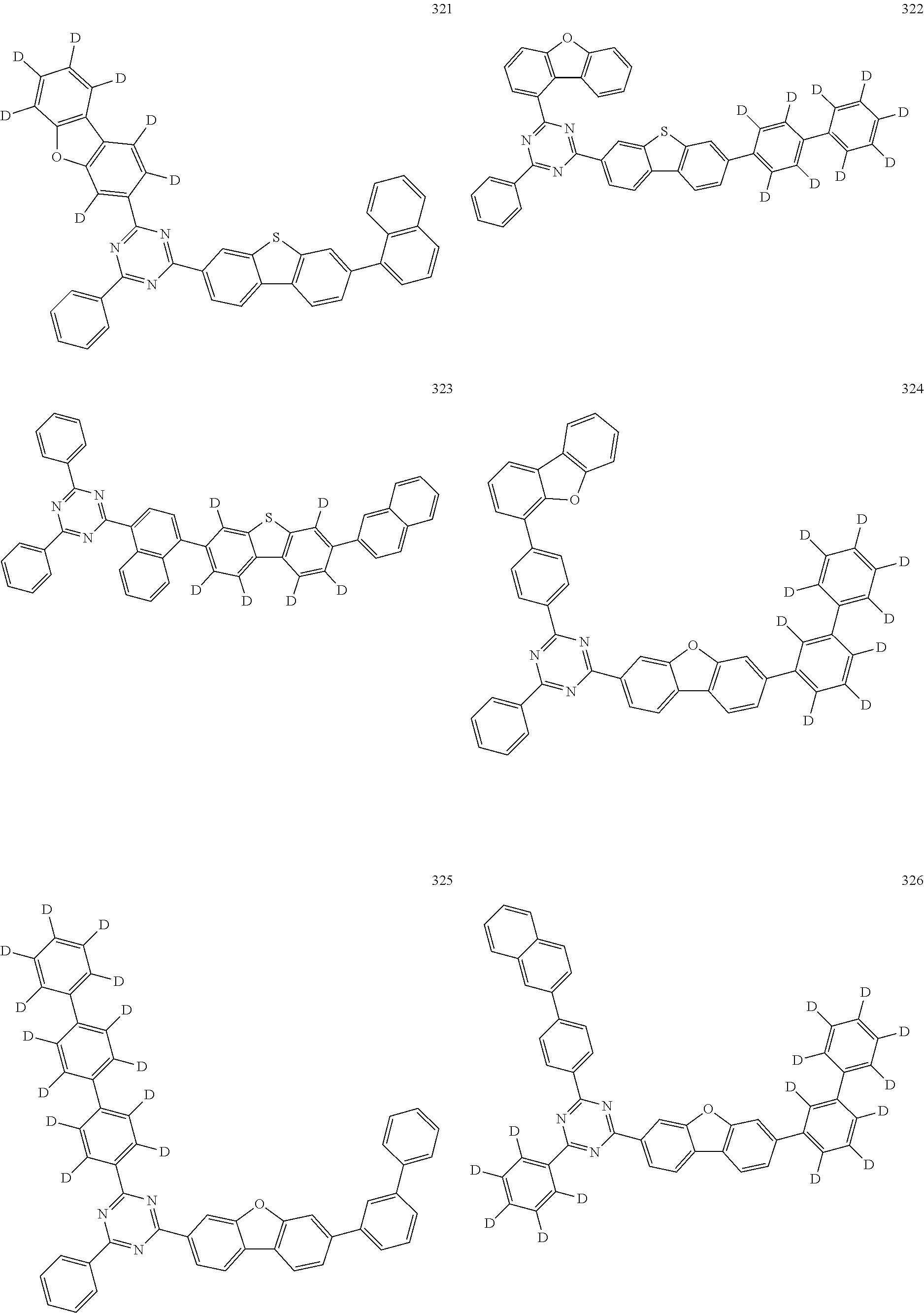

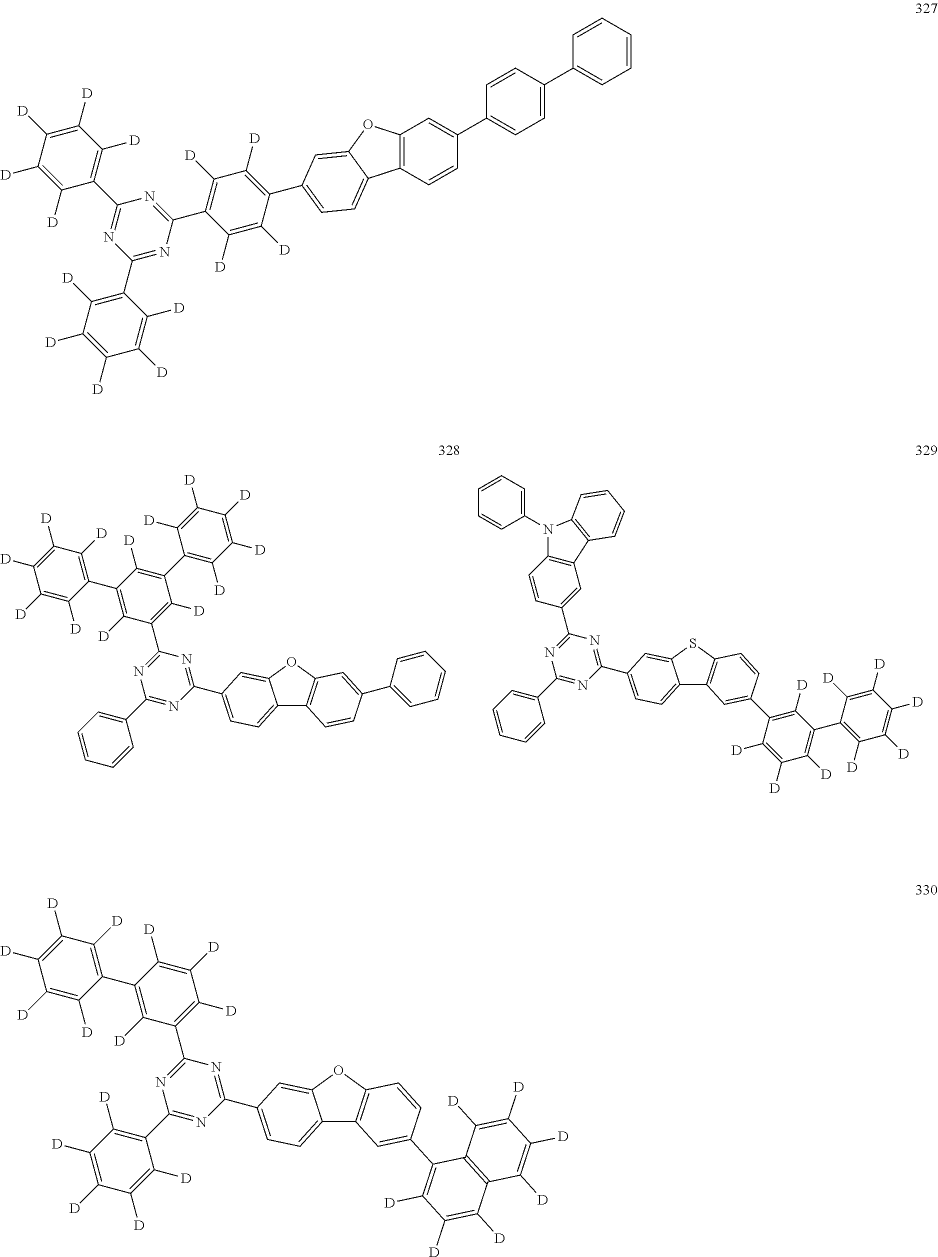

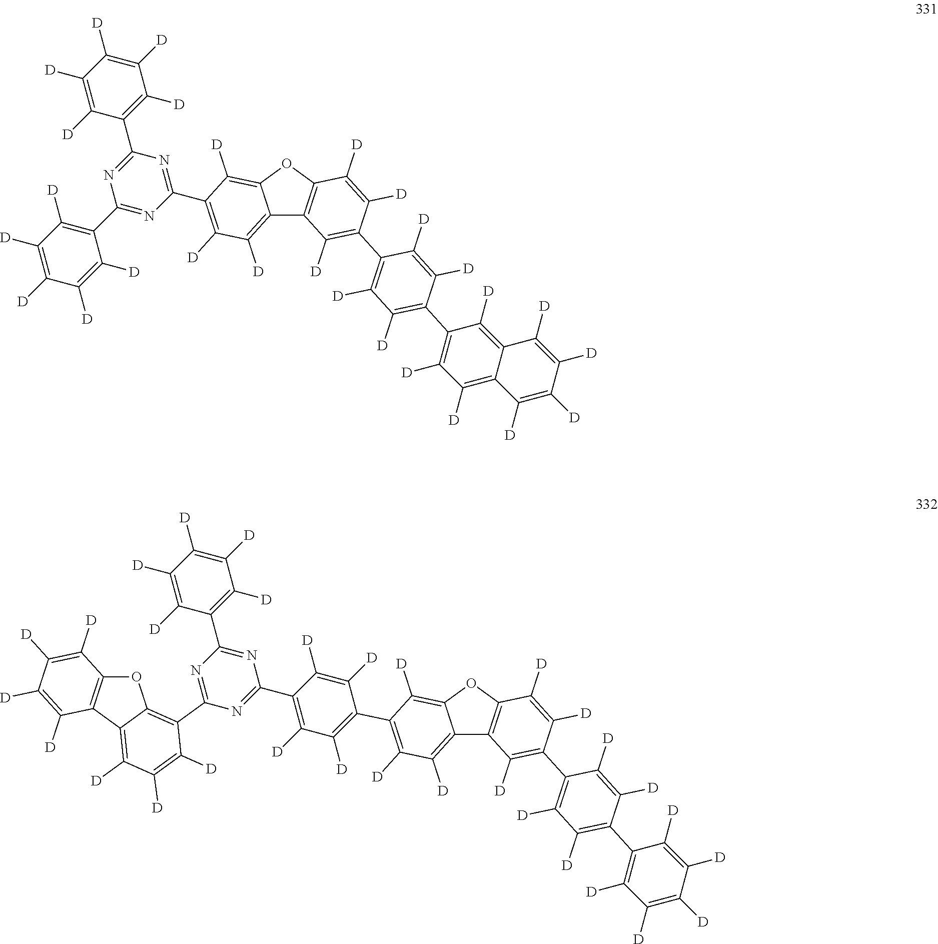

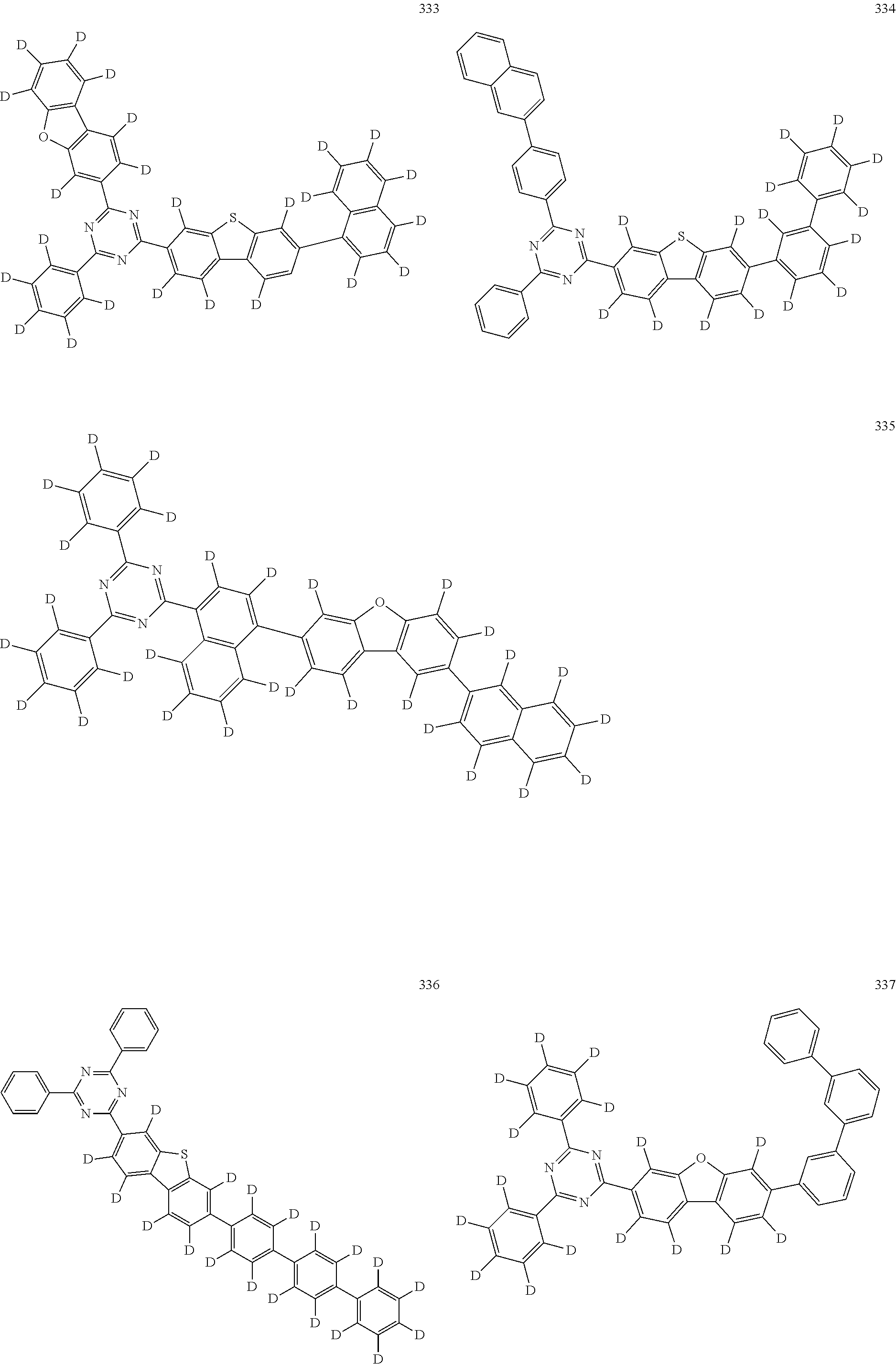

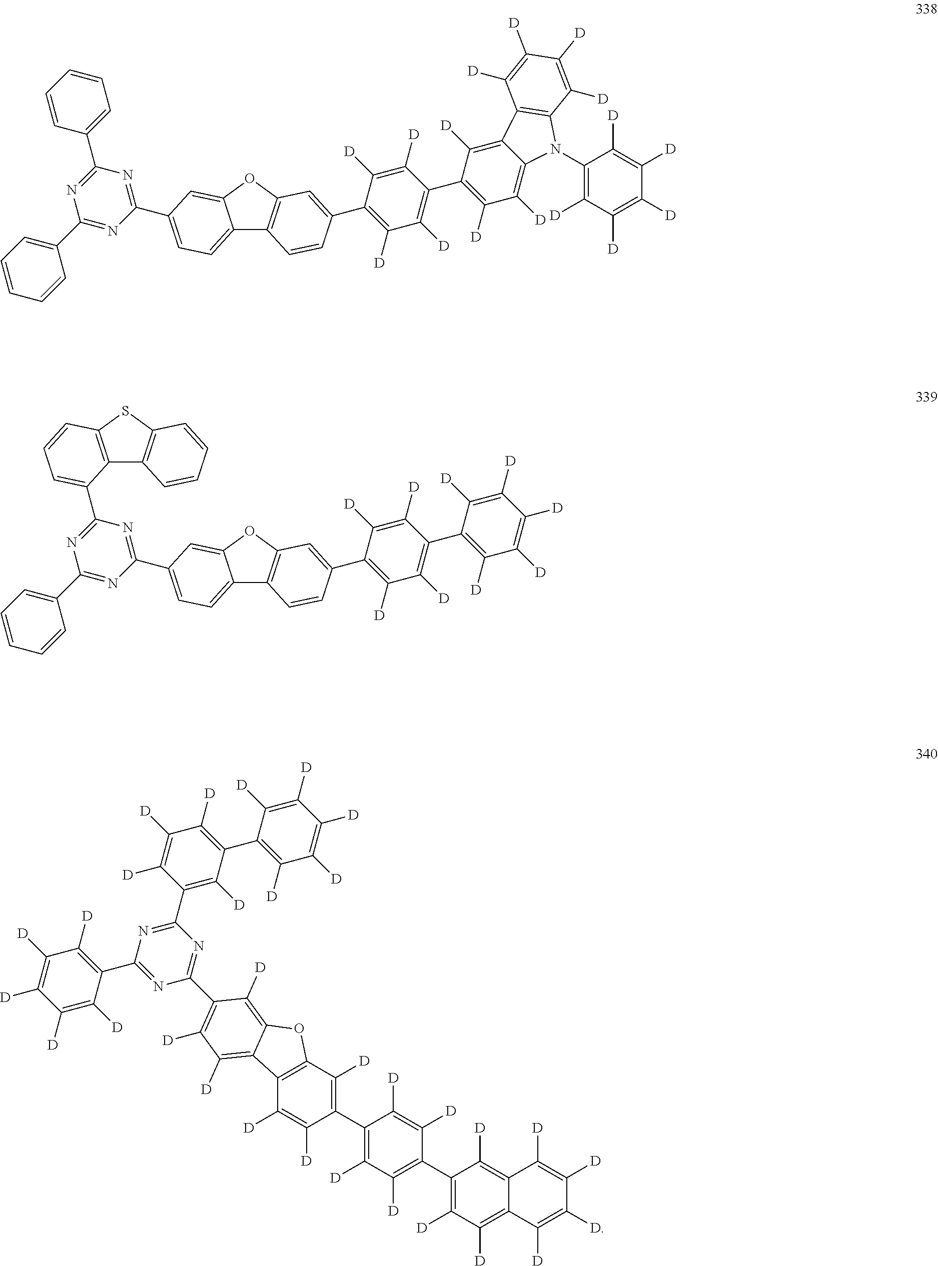

7. The organic light emitting device of claim 1, wherein the N-Het2 is represented by any one of the following Chemical Formulae 2-1 to 2-3: 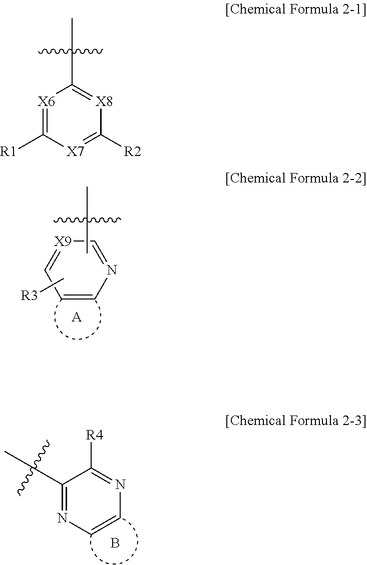
in Chemical Formulae 2-1 to 2-3,
X6 to X9 are each N or CR16,
at least one of X6 to X8 is N,
A and B are the same as or different from each other, and are each independently a substituted or unsubstituted monocyclic or polycyclic C6 to C60 aryl ring; or a substituted or unsubstituted monocyclic or polycyclic C2 to C60 hetero ring, and
R1 to R4 and R16 are the same as or different from each other, and are each independently hydrogen; deuterium; a halogen group; a cyano group; a substituted or unsubstituted C1 to C60 alkyl group; a substituted or unsubstituted C6 to C60 aryl group; or a substituted or unsubstituted C2 to C60 heteroaryl group.

8. The organic light emitting device of claim 1, wherein a deuterium content of the compound of Chemical Formula 2 is 0% or 1% to 100%.

9. The organic light emitting device of claim 1, wherein Chemical Formula 2 is represented by any one of the following compounds:

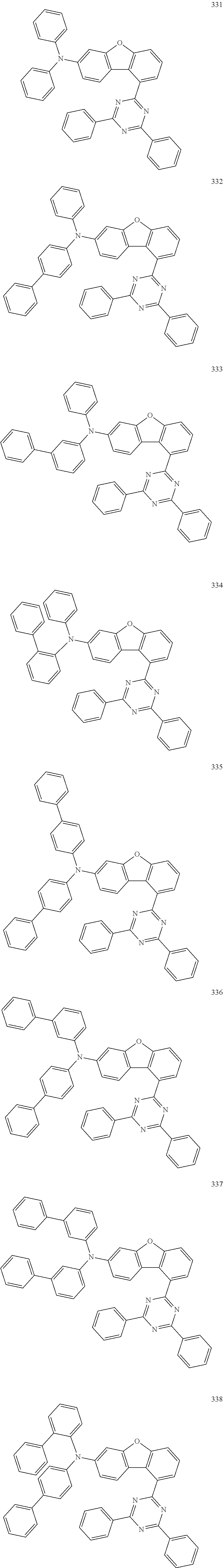

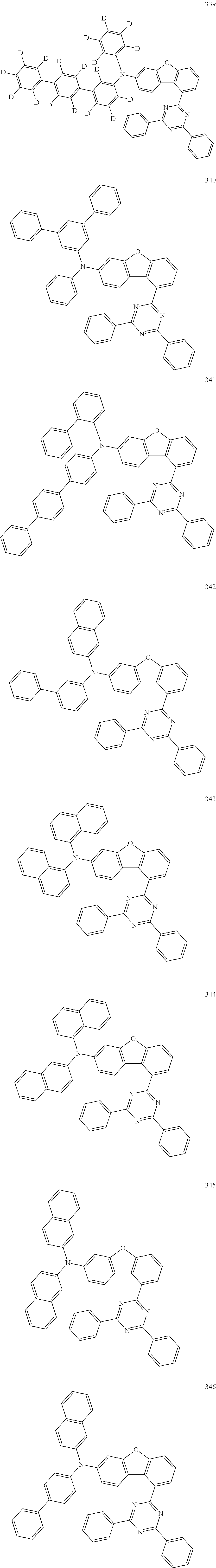

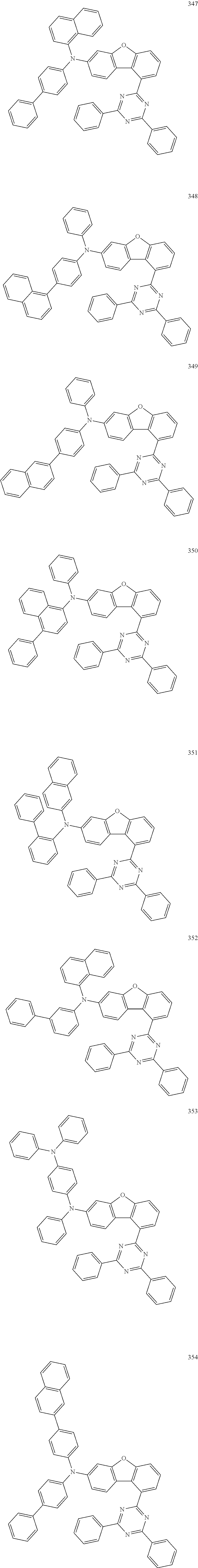

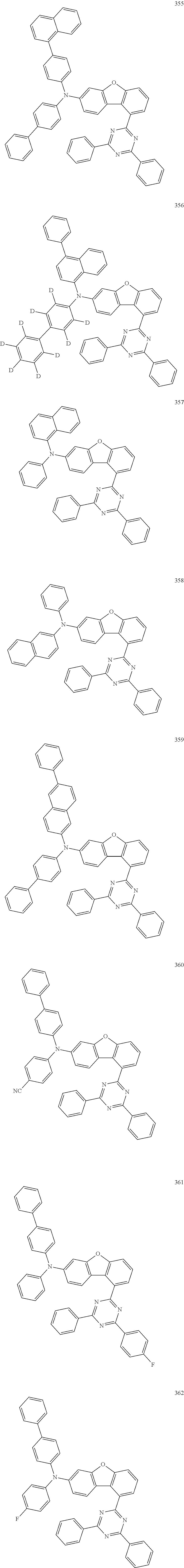

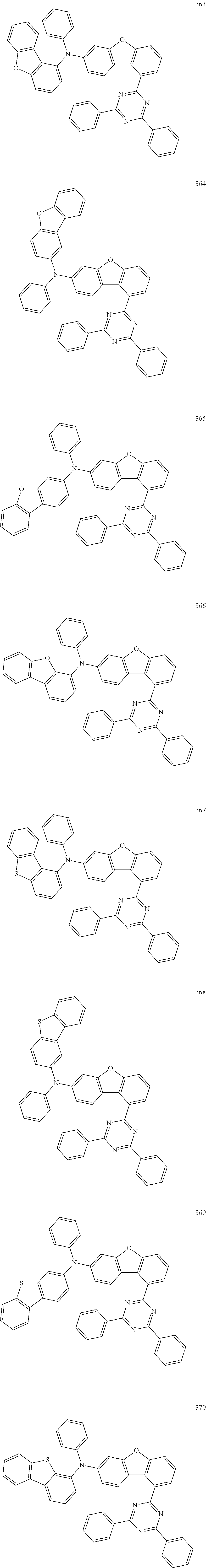

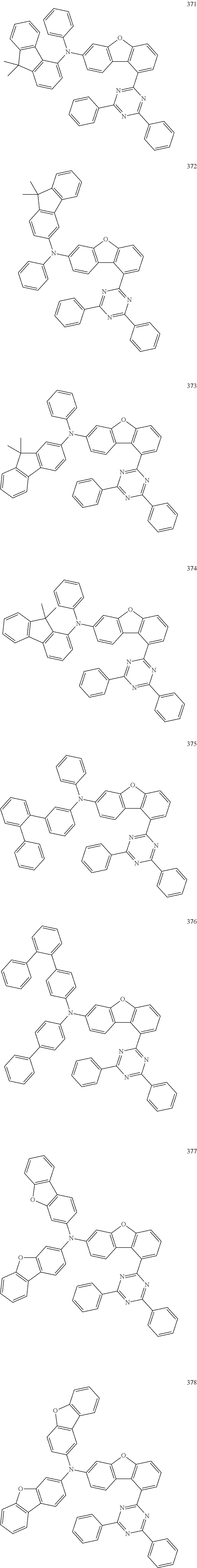

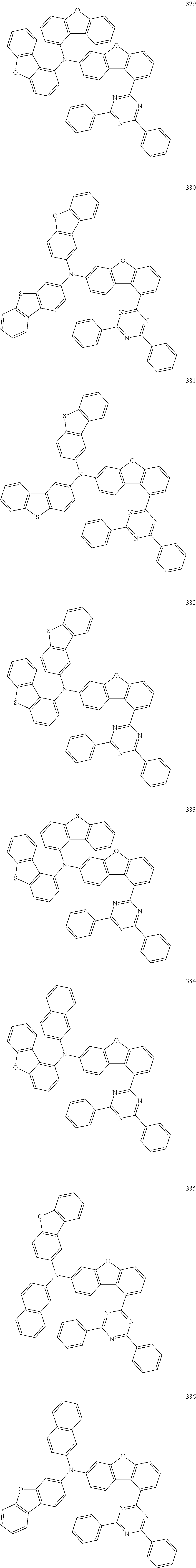

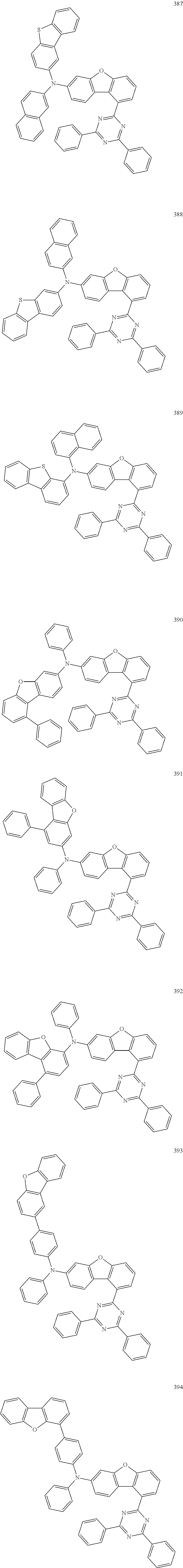

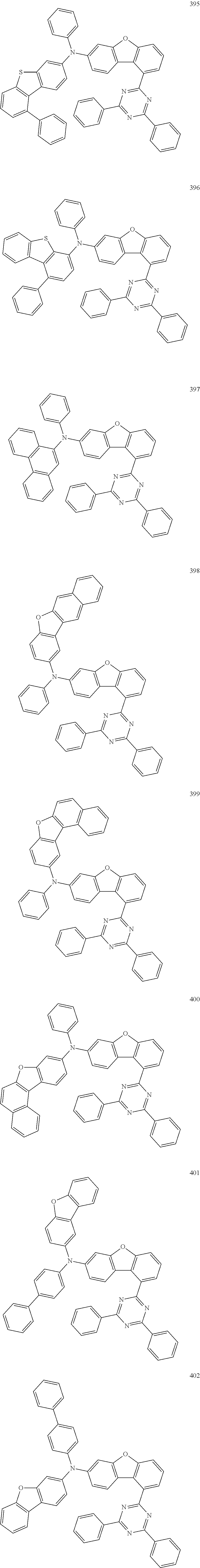

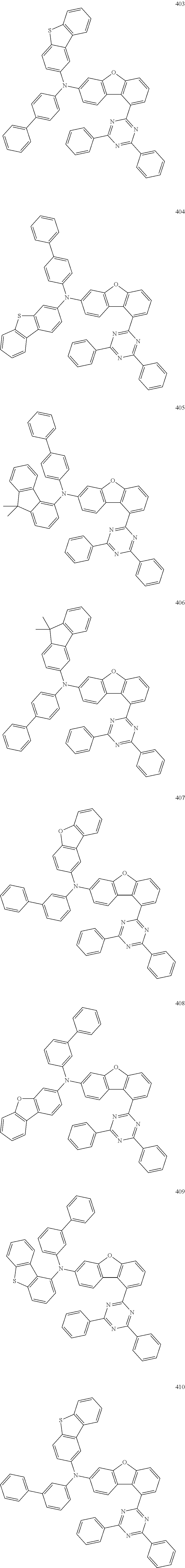

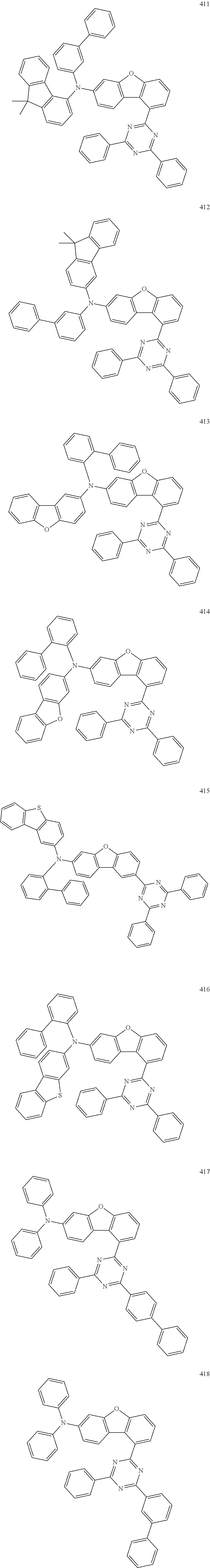

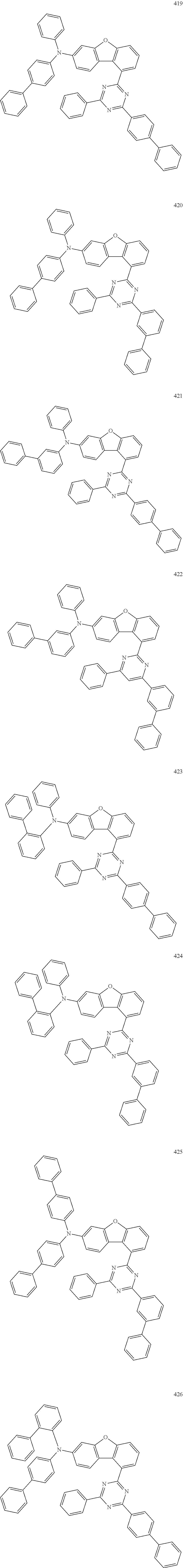

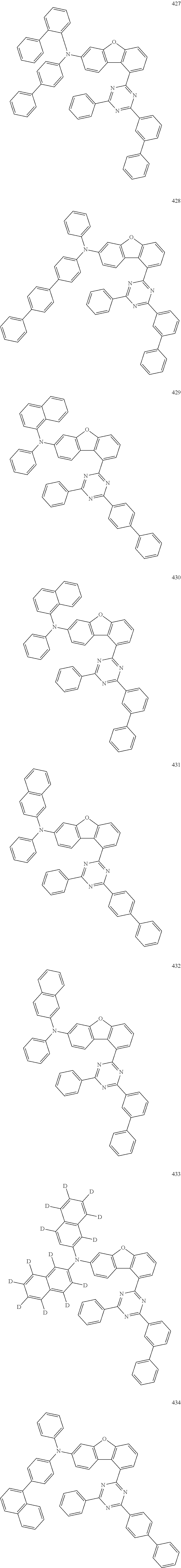

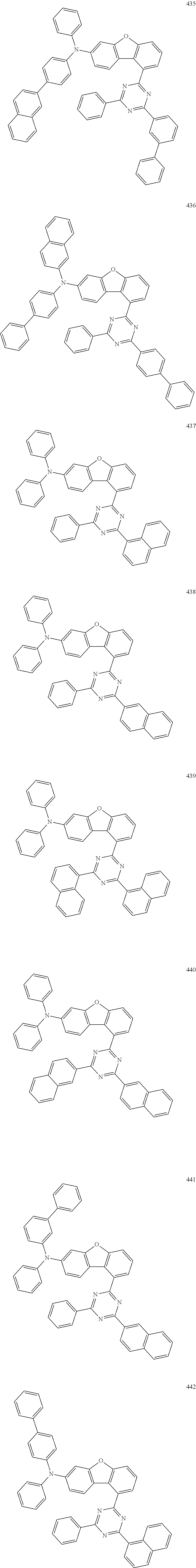

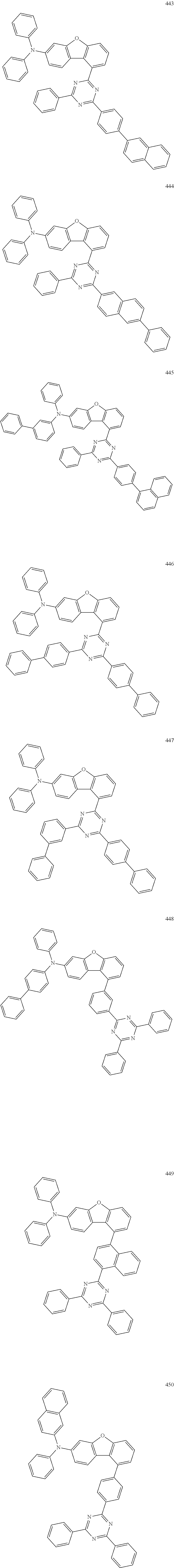

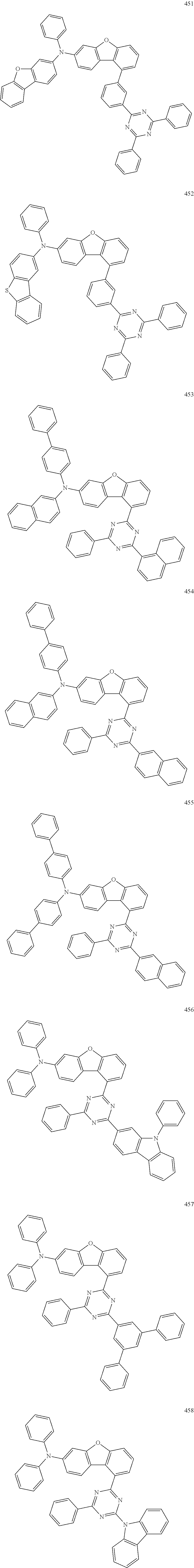

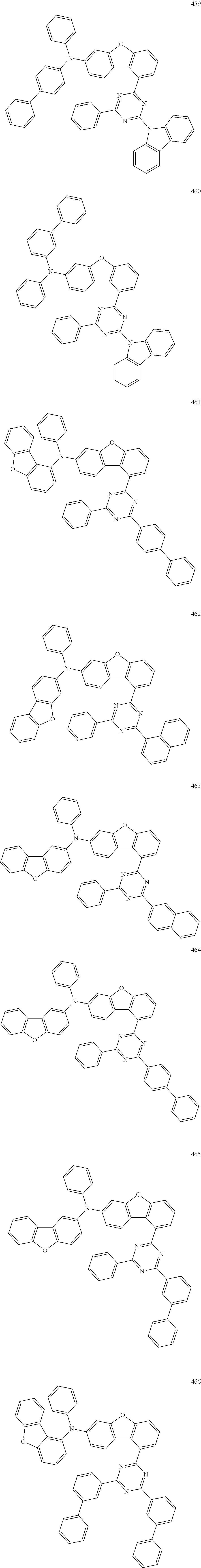

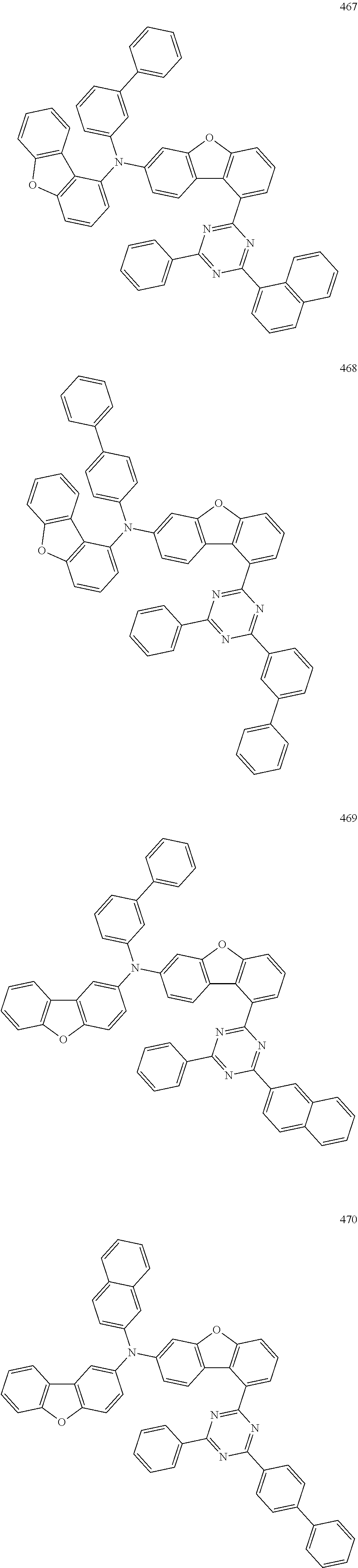 [SMILES: c1ccc(-c2cccc(N(c3ccc4c(c3)oc3cccc(-c5nc(-c6ccccc6)nc(-c6cccc7ccccc67)n5)c34)c3cccc4oc5ccccc5c34)c2)cc1 ; c1ccc(-c2ccc(N(c3ccc4c(c3)oc3cccc(-c5nc(-c6ccccc6)nc(-c6cccc(-c7ccccc7)c6)n5)c34)c3cccc4oc5ccccc5c34)cc2)cc1 ; c1ccc(-c2cccc(N(c3ccc4c(c3)oc3cccc(-c5nc(-c6ccccc6)nc(-c6ccc7ccccc7c6)n5)c34)c3ccc4oc5ccccc5c4c3)c2)cc1 ; c1ccc(-c2ccc(-c3nc(-c4ccccc4)nc(-c4cccc5oc6cc(N(c7ccc8ccccc8c7)c7ccc8oc9ccccc9c8c7)ccc6c45)n3)cc2)cc1]

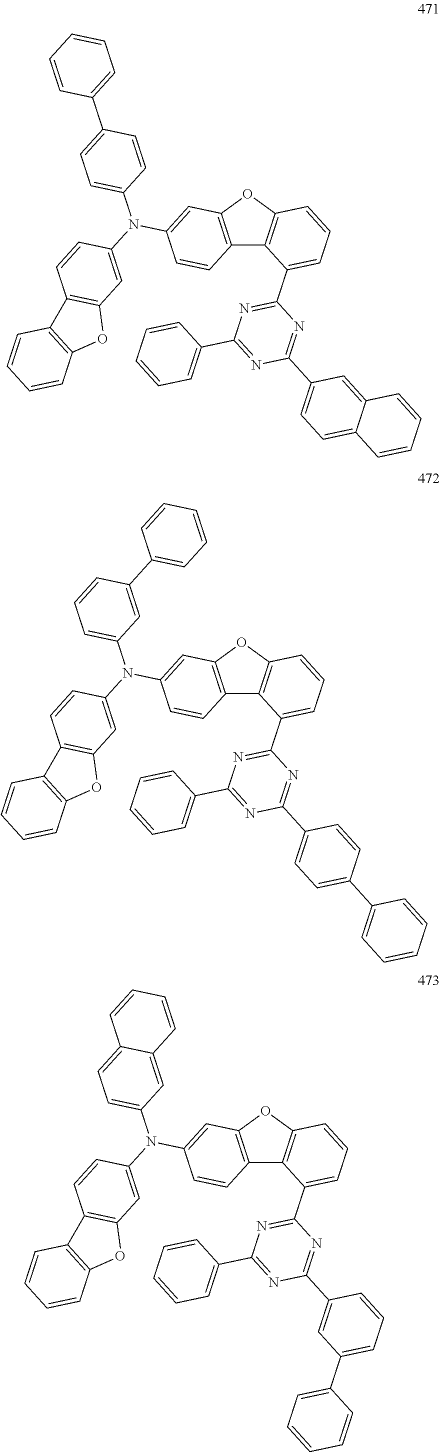 [SMILES: c1ccc(-c2ccc(N(c3ccc4c(c3)oc3ccccc34)c3ccc4c(c3)oc3cccc(-c5nc(-c6ccccc6)nc(-c6ccc7ccccc7c6)n5)c34)cc2)cc1 ; c1ccc(-c2ccc(-c3nc(-c4ccccc4)nc(-c4cccc5oc6cc(N(c7cccc(-c8ccccc8)c7)c7ccc8c(c7)oc7ccccc78)ccc6c45)n3)cc2)cc1 ; c1ccc(-c2cccc(-c3nc(-c4ccccc4)nc(-c4cccc5oc6cc(N(c7ccc8ccccc8c7)c7ccc8c(c7)oc7ccccc78)ccc6c45)n3)c2)cc1]

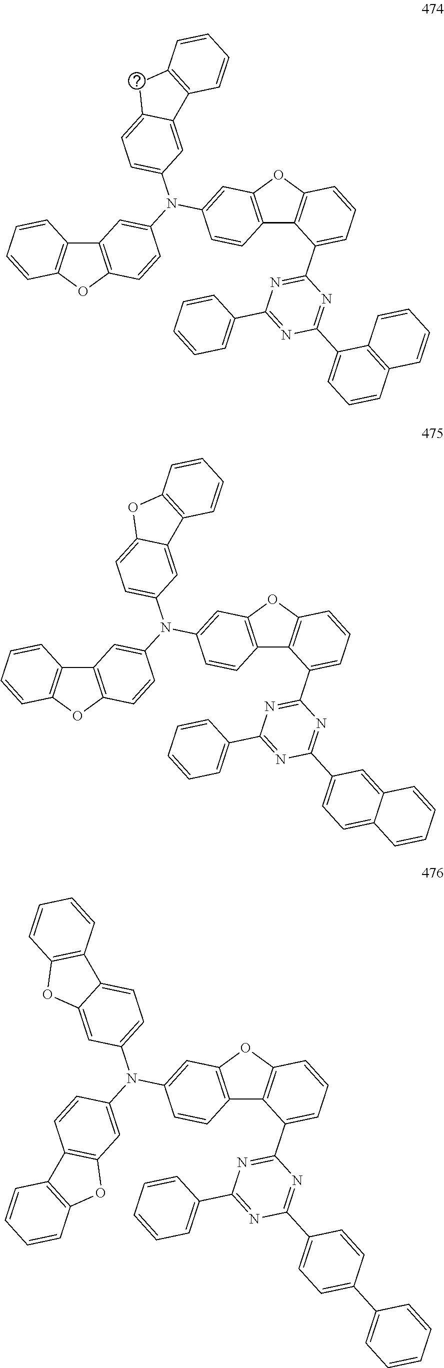 [SMILES: c1ccc(-c2nc(-c3cccc4ccccc34)nc(-c3cccc4oc5cc(N(c6ccc7c(c6)-c6ccccc6C7)c6ccc7oc8ccccc8c7c6)ccc5c34)n2)cc1 ; c1ccc(-c2nc(-c3ccc4ccccc4c3)nc(-c3cccc4oc5cc(N(c6ccc7oc8ccccc8c7c6)c6ccc7oc8ccccc8c7c6)ccc5c34)n2)cc1 ; c1ccc(-c2ccc(-c3nc(-c4ccccc4)nc(-c4cccc5oc6cc(N(c7ccc8c(c7)oc7ccccc78)c7ccc8c(c7)oc7ccccc78)ccc6c45)n3)cc2)cc1]

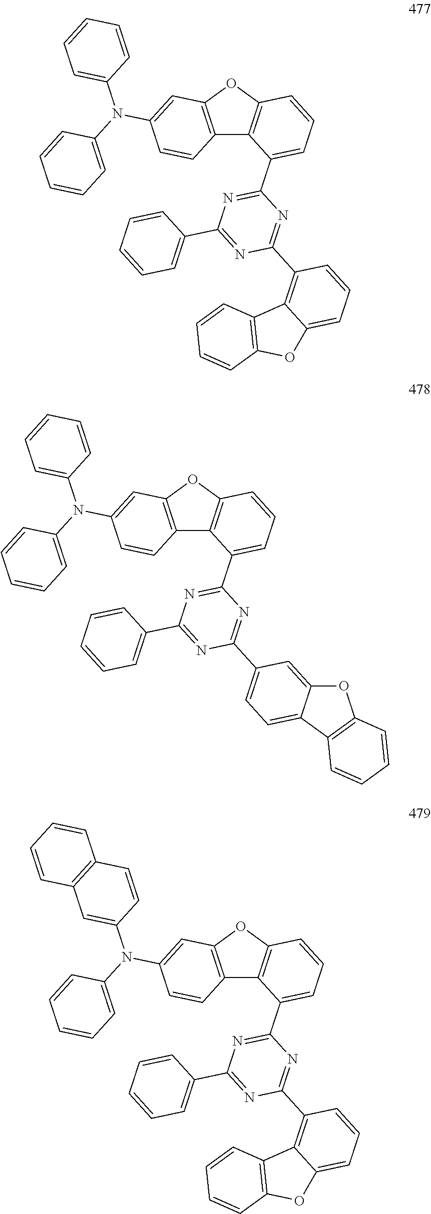 [SMILES: c1ccc(-c2nc(-c3cccc4oc5ccccc5c34)nc(-c3cccc4oc5cc(N(c6ccccc6)c6ccccc6)ccc5c34)n2)cc1 ; c1ccc(-c2nc(-c3ccc4c(c3)oc3ccccc34)nc(-c3cccc4oc5cc(N(c6ccccc6)c6ccccc6)ccc5c34)n2)cc1 ; c1ccc(-c2nc(-c3cccc4oc5ccccc5c34)nc(-c3cccc4oc5cc(N(c6ccccc6)c6ccc7ccccc7c6)ccc5c34)n2)cc1]

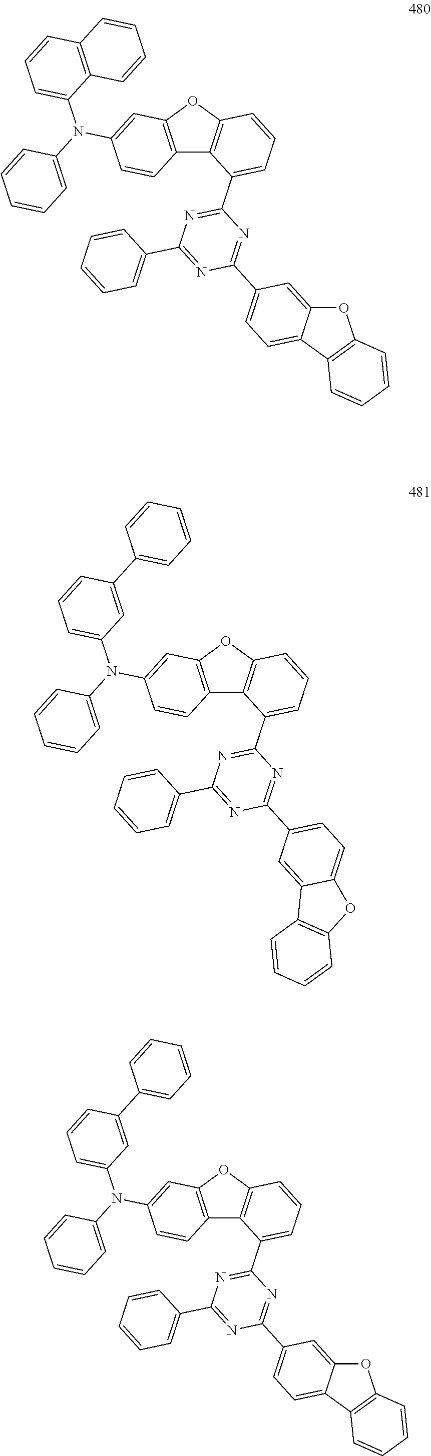 [SMILES: c1ccc(-c2nc(-c3ccc4c(c3)oc3ccccc34)nc(-c3cccc4oc5cc(N(c6ccccc6)c6cccc7ccccc67)ccc5c34)n2)cc1 ; c1ccc(-c2cccc(N(c3ccccc3)c3ccc4c(c3)oc3cccc(-c5nc(-c6ccccc6)nc(-c6ccc7oc8ccccc8c7c6)n5)c34)c2)cc1 ; c1ccc(-c2cccc(N(c3ccccc3)c3ccc4c(c3)oc3cccc(-c5nc(-c6ccccc6)nc(-c6ccc7c(c6)oc6ccccc67)n5)c34)c2)cc1]

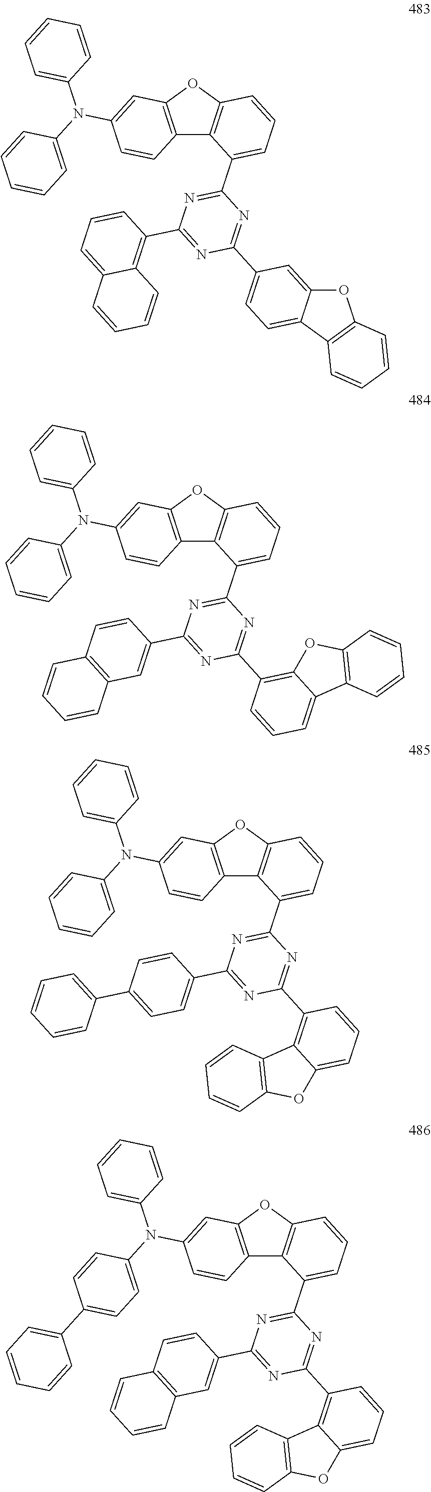

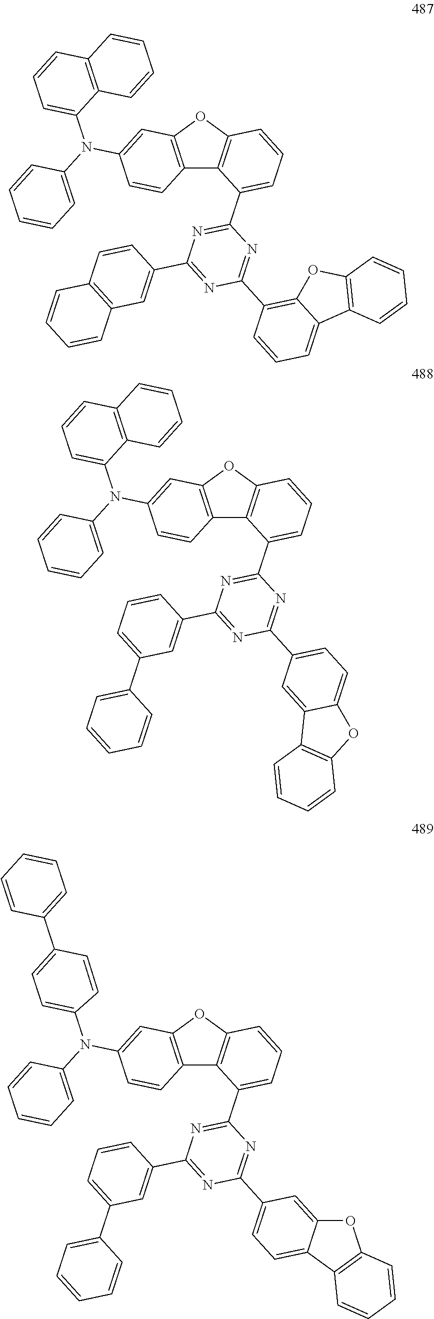 [SMILES: c1ccc(N(c2ccc3c(c2)oc2cccc(-c4nc(-c5ccc6ccccc6c5)nc(-c5cccc6c5oc5ccccc56)n4)c23)c2cccc3ccccc23)cc1 ; c1ccc(-c2cccc(-c3nc(-c4ccc5oc6ccccc6c5c4)nc(-c4cccc5oc6cc(N(c7ccccc7)c7cccc8ccccc78)ccc6c45)n3)c2)cc1 ; c1ccc(-c2ccc(N(c3ccccc3)c3ccc4c(c3)oc3cccc(-c5nc(-c6cccc(-c7ccccc7)c6)nc(-c6ccc7c(c6)oc6ccccc67)n5)c34)cc2)cc1]

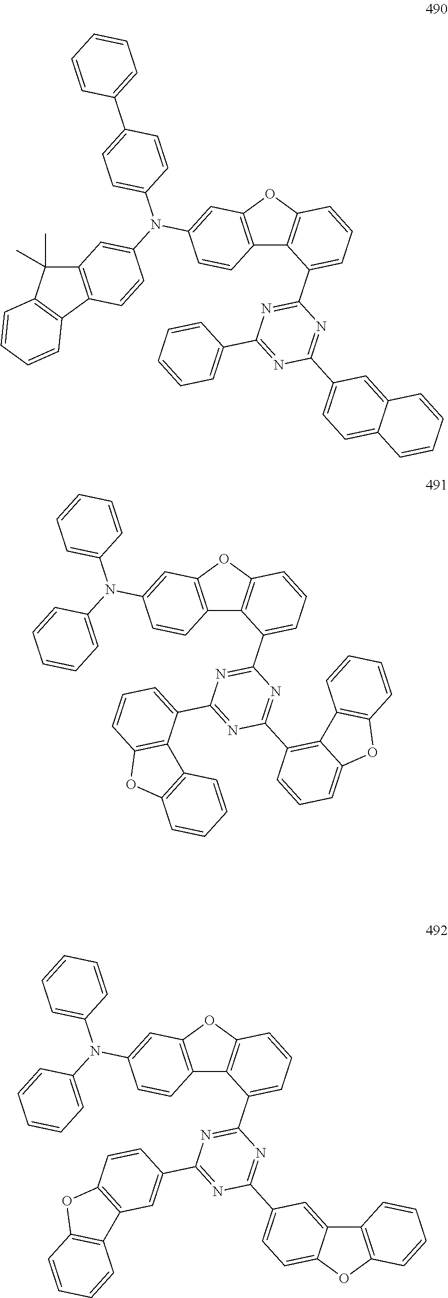 [SMILES: CC1(C)c2ccccc2-c2ccc(N(c3ccc(-c4ccccc4)cc3)c3ccc4c(c3)oc3cccc(-c5nc(-c6ccccc6)nc(-c6ccc7ccccc7c6)n5)c34)cc21 ; c1ccc(N(c2ccccc2)c2ccc3c(c2)oc2cccc(-c4nc(-c5cccc6oc7ccccc7c56)nc(-c5cccc6oc7ccccc7c56)n4)c23)cc1 ; c1ccc(N(c2ccccc2)c2ccc3c(c2)oc2cccc(-c4nc(-c5ccc6oc7ccccc7c6c5)nc(-c5ccc6oc7ccccc7c6c5)n4)c23)cc1]

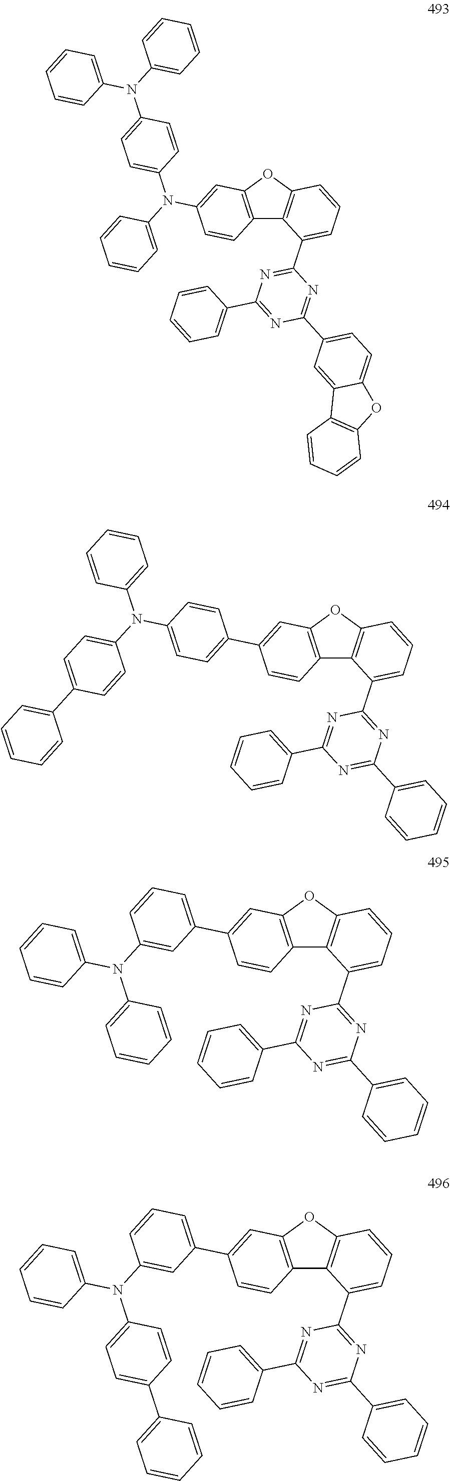 [SMILES: c1ccc(-c2nc(-c3ccc4oc5ccccc5c4c3)nc(-c3cccc4oc5cc(N(c6ccccc6)c6ccc(N(c7ccccc7)c7ccccc7)cc6)ccc5c34)n2)cc1 ; c1ccc(-c2ccc(N(c3ccccc3)c3ccc(-c4ccc5c(c4)oc4cccc(-c6nc(-c7ccccc7)nc(-c7ccccc7)n6)c45)cc3)cc2)cc1 ; c1ccc(-c2nc(-c3ccccc3)nc(-c3cccc4oc5cc(-c6cccc(N(c7ccccc7)c7ccccc7)c6)ccc5c34)n2)cc1 ; c1ccc(-c2ccc(N(c3ccccc3)c3cccc(-c4ccc5c(c4)oc4cccc(-c6nc(-c7ccccc7)nc(-c7ccccc7)n6)c45)c3)cc2)cc1]

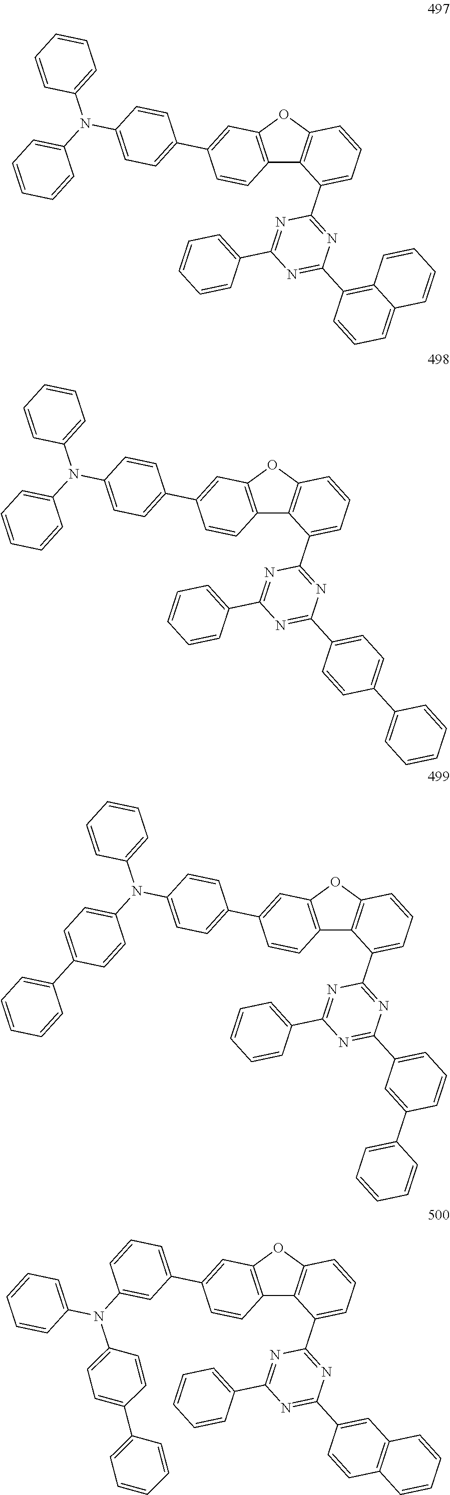 [SMILES: c1ccc(-c2nc(-c3cccc4ccccc34)nc(-c3cccc4oc5cc(-c6ccc(N(c7ccccc7)c7ccccc7)cc6)ccc5c34)n2)cc1 ; c1ccc(-c2ccc(-c3nc(-c4ccccc4)nc(-c4cccc5oc6cc(-c7ccc(N(c8ccccc8)c8ccccc8)cc7)ccc6c45)n3)cc2)cc1 ; c1ccc(-c2ccc(N(c3ccccc3)c3ccc(-c4ccc5c(c4)oc4cccc(-c6nc(-c7ccccc7)nc(-c7cccc(-c8ccccc8)c7)n6)c45)cc3)cc2)cc1 ; c1ccc(-c2ccc(N(c3ccccc3)c3cccc(-c4ccc5c(c4)oc4cccc(-c6nc(-c7ccccc7)nc(-c7ccc8ccccc8c7)n6)c45)c3)cc2)cc1]

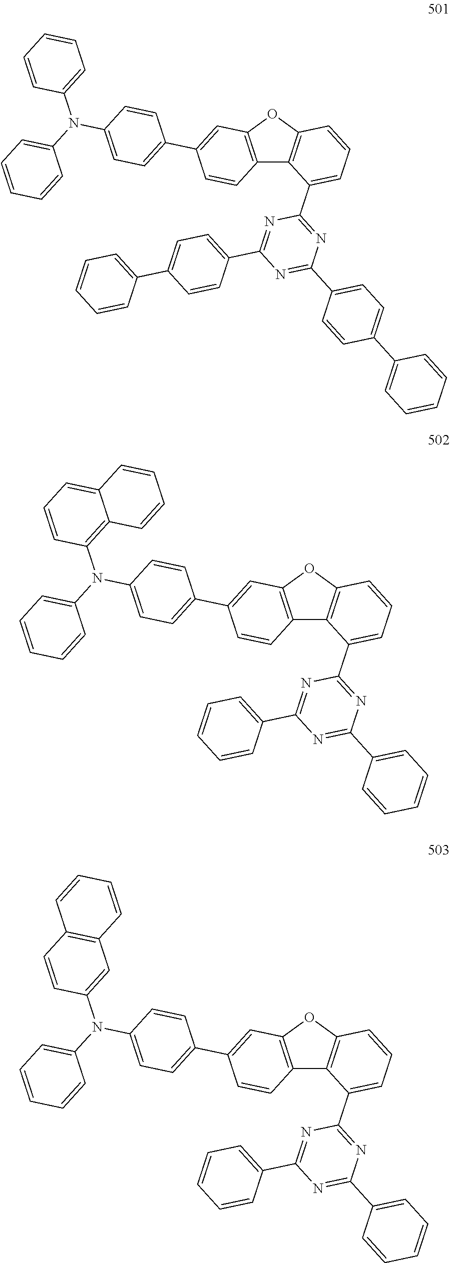 [SMILES: c1ccc(-c2ccc(-c3nc(-c4ccc(-c5ccccc5)cc4)nc(-c4cccc5oc6cc(-c7ccc(N(c8ccccc8)c8ccccc8)cc7)ccc6c45)n3)cc2)cc1 ; c1ccc(-c2nc(-c3ccccc3)nc(-c3cccc4oc5cc(-c6ccc(N(c7ccccc7)c7cccc8ccccc78)cc6)ccc5c34)n2)cc1 ; c1ccc(-c2nc(-c3ccccc3)nc(-c3cccc4oc5cc(-c6ccc(N(c7ccccc7)c7ccc8ccccc8c7)cc6)ccc5c34)n2)cc1]

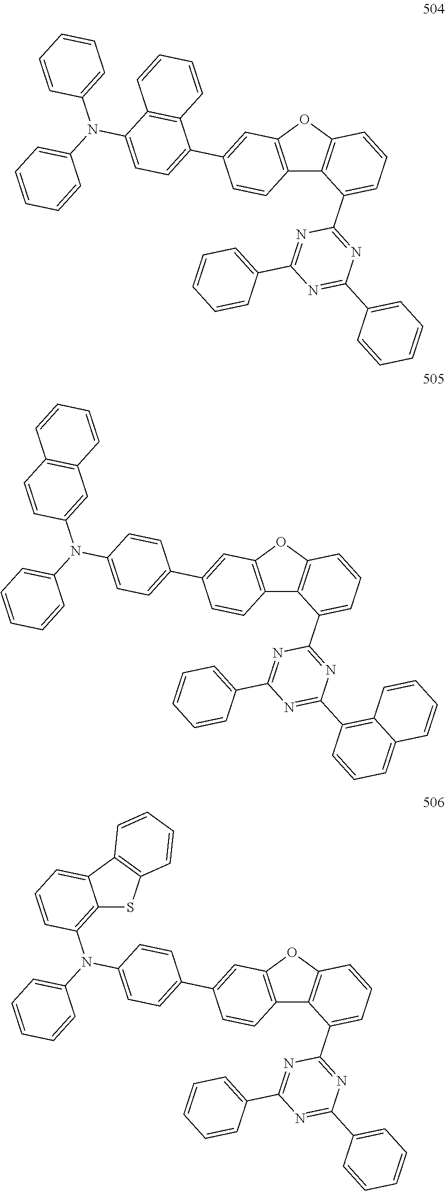 [SMILES: c1ccc(-c2nc(-c3ccccc3)nc(-c3cccc4oc5cc(-c6ccc(N(c7ccccc7)c7ccccc7)c7ccccc67)ccc5c34)n2)cc1 ; c1ccc(-c2nc(-c3cccc4ccccc34)nc(-c3cccc4oc5cc(-c6ccc(N(c7ccccc7)c7ccc8ccccc8c7)cc6)ccc5c34)n2)cc1 ; c1ccc(-c2nc(-c3ccccc3)nc(-c3cccc4oc5cc(-c6ccc(N(c7ccccc7)c7cccc8c7sc7ccccc78)cc6)ccc5c34)n2)cc1]

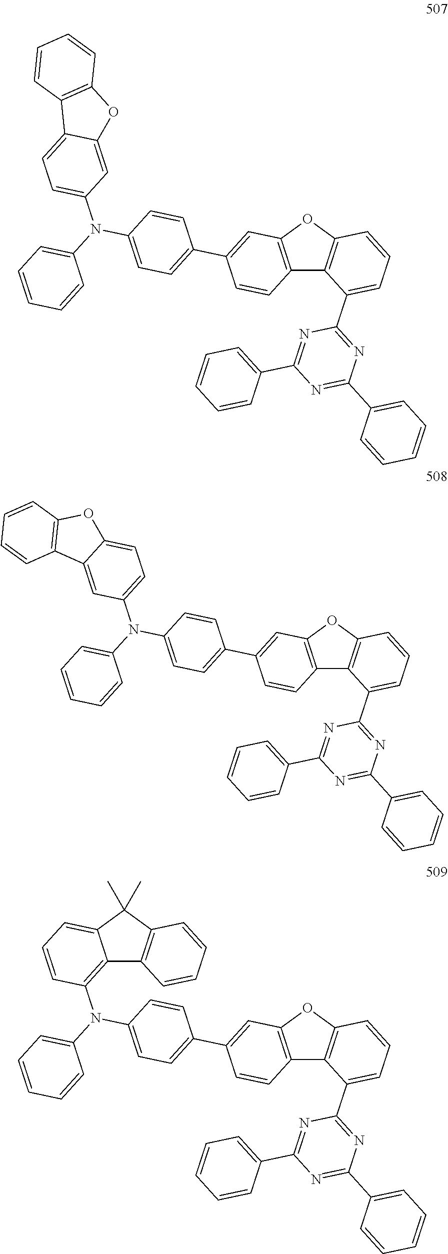 [SMILES: c1ccc(-c2nc(-c3ccccc3)nc(-c3cccc4oc5cc(-c6ccc(N(c7ccccc7)c7ccc8c(c7)oc7ccccc78)cc6)ccc5c34)n2)cc1 ; c1ccc(-c2nc(-c3ccccc3)nc(-c3cccc4oc5cc(-c6ccc(N(c7ccccc7)c7ccc8oc9ccccc9c8c7)cc6)ccc5c34)n2)cc1 ; CC1(C)c2ccccc2-c2c(N(c3ccccc3)c3ccc(-c4ccc5c(c4)oc4cccc(-c6nc(-c7ccccc7)nc(-c7ccccc7)n6)c45)cc3)cccc21]

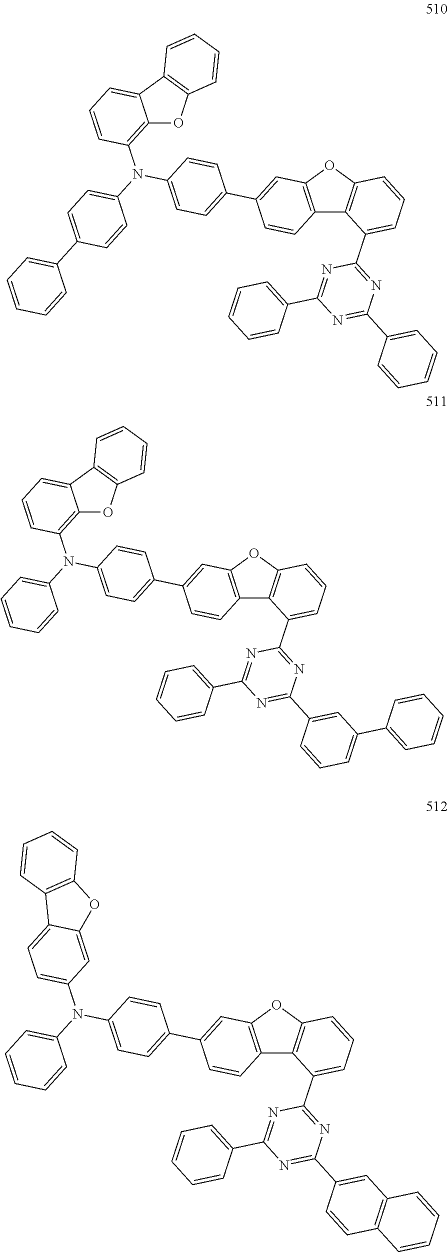 [SMILES: c1ccc(-c2ccc(N(c3ccc(-c4ccc5c(c4)oc4cccc(-c6nc(-c7ccccc7)nc(-c7ccccc7)n6)c45)cc3)c3cccc4c3oc3ccccc34)cc2)cc1 ; c1ccc(-c2cccc(-c3nc(-c4ccccc4)nc(-c4cccc5oc6cc(-c7ccc(N(c8ccccc8)c8cccc9c8oc8ccccc89)cc7)ccc6c45)n3)c2)cc1 ; c1ccc(-c2nc(-c3ccc4ccccc4c3)nc(-c3cccc4oc5cc(-c6ccc(N(c7ccccc7)c7ccc8c(c7)oc7ccccc78)cc6)ccc5c34)n2)cc1]

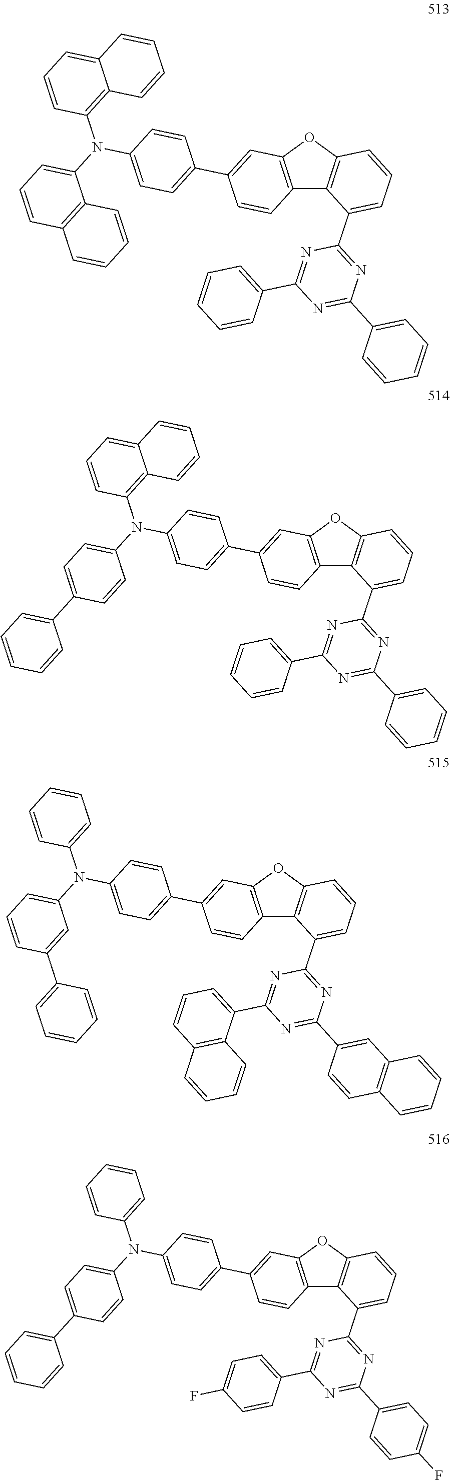 [SMILES: c1ccc(-c2nc(-c3ccccc3)nc(-c3cccc4oc5cc(-c6ccc(N(c7cccc8ccccc78)c7cccc8ccccc78)cc6)ccc5c34)n2)cc1 ; c1ccc(-c2ccc(N(c3ccc(-c4ccc5c(c4)oc4cccc(-c6nc(-c7ccccc7)nc(-c7ccccc7)n6)c45)cc3)c3cccc4ccccc34)cc2)cc1 ; c1ccc(-c2cccc(N(c3ccccc3)c3ccc(-c4ccc5c(c4)oc4cccc(-c6nc(-c7ccc8ccccc8c7)nc(-c7cccc8ccccc78)n6)c45)cc3)c2)cc1 ; Fc1ccc(-c2nc(-c3ccc(F)cc3)nc(-c3cccc4oc5cc(-c6ccc(N(c7ccccc7)c7ccc(-c8ccccc8)cc7)cc6)ccc5c34)n2)cc1]

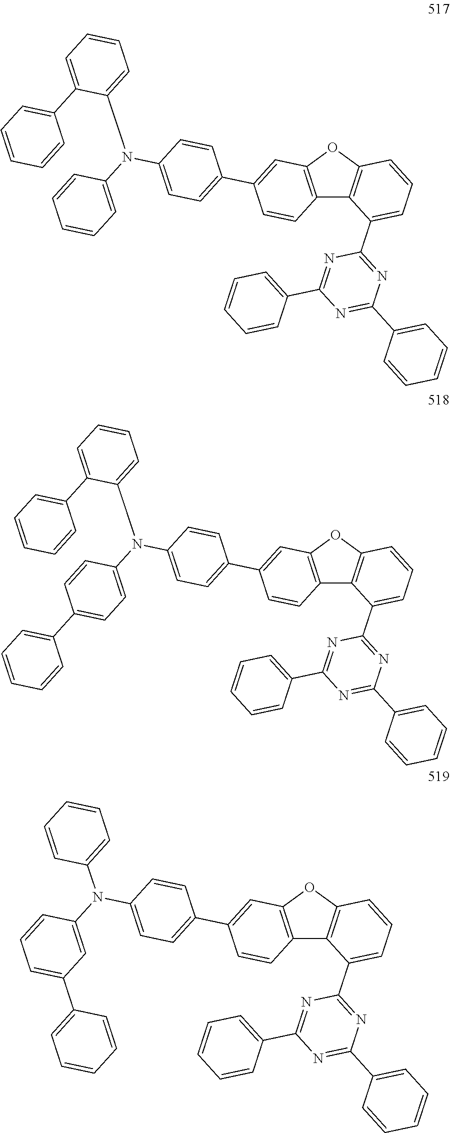 [SMILES: c1ccc(-c2nc(-c3ccccc3)nc(-c3cccc4oc5cc(-c6ccc(N(c7ccccc7)c7ccccc7-c7ccccc7)cc6)ccc5c34)n2)cc1 ; c1ccc(-c2ccc(N(c3ccc(-c4ccc5c(c4)oc4cccc(-c6nc(-c7ccccc7)nc(-c7ccccc7)n6)c45)cc3)c3ccccc3-c3ccccc3)cc2)cc1 ; c1ccc(-c2cccc(N(c3ccccc3)c3ccc(-c4ccc5c(c4)oc4cccc(-c6nc(-c7ccccc7)nc(-c7ccccc7)n6)c45)cc3)c2)cc1]

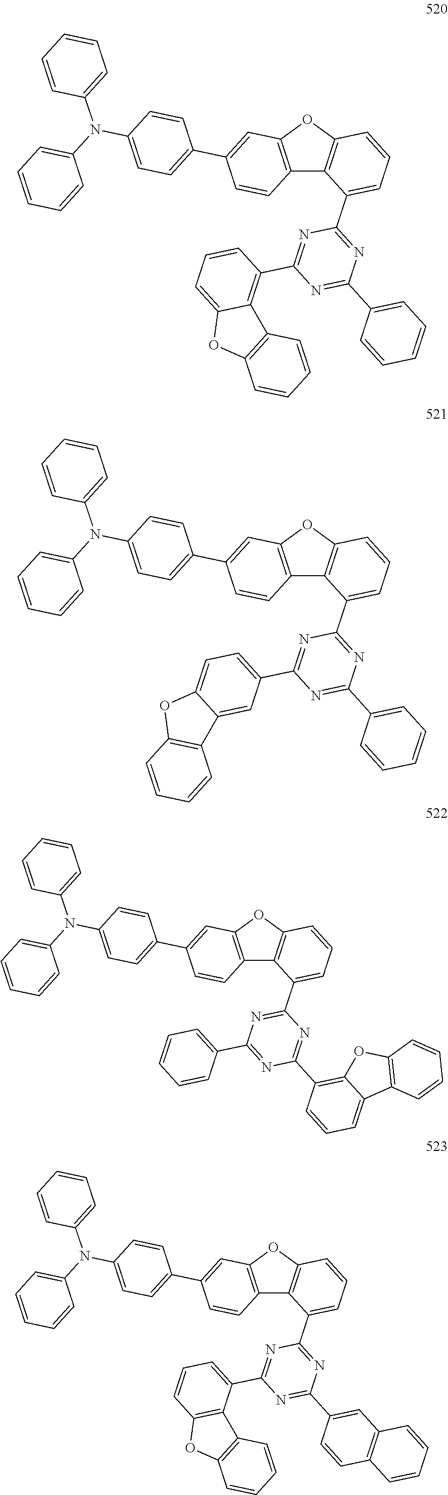 [SMILES: c1ccc(-c2nc(-c3cccc4oc5ccccc5c34)nc(-c3cccc4oc5cc(-c6ccc(N(c7ccccc7)c7ccccc7)cc6)ccc5c34)n2)cc1 ; c1ccc(-c2nc(-c3ccc4oc5ccccc5c4c3)nc(-c3cccc4oc5cc(-c6ccc(N(c7ccccc7)c7ccccc7)cc6)ccc5c34)n2)cc1 ; c1ccc(-c2nc(-c3cccc4c3oc3ccccc34)nc(-c3cccc4oc5cc(-c6ccc(N(c7ccccc7)c7ccccc7)cc6)ccc5c34)n2)cc1 ; c1ccc(N(c2ccccc2)c2ccc(-c3ccc4c(c3)oc3cccc(-c5nc(-c6ccc7ccccc7c6)nc(-c6cccc7oc8ccccc8c67)n5)c34)cc2)cc1]

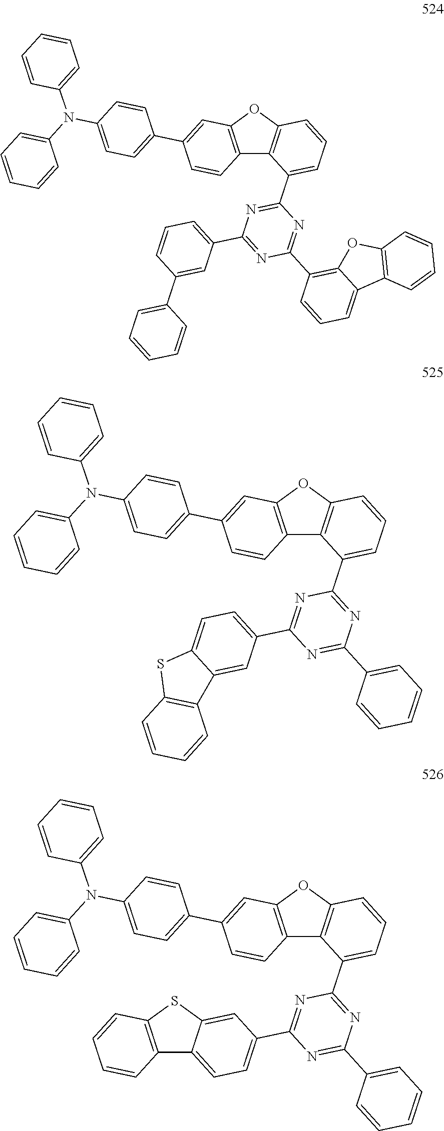 [SMILES: c1ccc(-c2cccc(-c3nc(-c4cccc5c4oc4ccccc45)nc(-c4cccc5oc6cc(-c7ccc(N(c8ccccc8)c8ccccc8)cc7)ccc6c45)n3)c2)cc1 ; c1ccc(-c2nc(-c3ccc4sc5ccccc5c4c3)nc(-c3cccc4oc5cc(-c6ccc(N(c7ccccc7)c7ccccc7)cc6)ccc5c34)n2)cc1 ; c1ccc(-c2nc(-c3ccc4c(c3)sc3ccccc34)nc(-c3cccc4oc5cc(-c6ccc(N(c7ccccc7)c7ccccc7)cc6)ccc5c34)n2)cc1]

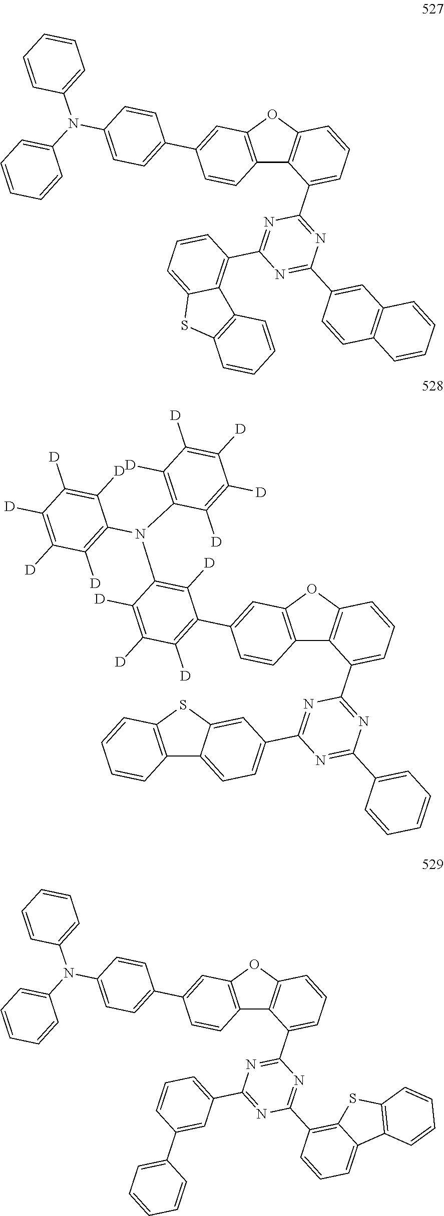 [SMILES: c1ccc(N(c2ccccc2)c2ccc(-c3ccc4c(c3)oc3cccc(-c5nc(-c6ccc7ccccc7c6)nc(-c6cccc7sc8ccccc8c67)n5)c34)cc2)cc1 ; [2H]c1c([2H])c([2H])c(N(c2c([2H])c([2H])c([2H])c([2H])c2[2H])c2c([2H])c([2H])c([2H])c(-c3ccc4c(c3)oc3cccc(-c5nc(-c6ccccc6)nc(-c6ccc7c(c6)sc6ccccc67)n5)c34)c2[2H])c([2H])c1[2H] ; c1ccc(-c2cccc(-c3nc(-c4cccc5c4sc4ccccc45)nc(-c4cccc5oc6cc(-c7ccc(N(c8ccccc8)c8ccccc8)cc7)ccc6c45)n3)c2)cc1]

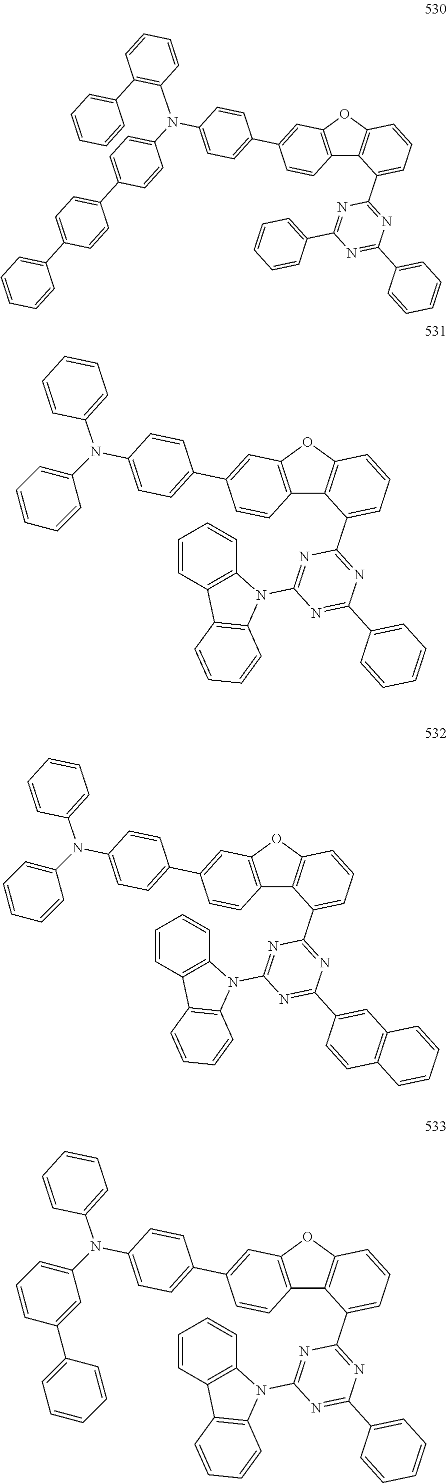 [SMILES: c1ccc(-c2ccc(-c3ccc(N(c4ccc(-c5ccc6c(c5)oc5cccc(-c7nc(-c8ccccc8)nc(-c8ccccc8)n7)c56)cc4)c4ccccc4-c4ccccc4)cc3)cc2)cc1 ; c1ccc(-c2nc(-c3cccc4oc5cc(-c6ccc(N(c7ccccc7)c7ccccc7)cc6)ccc5c34)nc(-n3c4ccccc4c4ccccc43)n2)cc1 ; c1ccc(N(c2ccccc2)c2ccc(-c3ccc4c(c3)oc3cccc(-c5nc(-c6ccc7ccccc7c6)nc(-n6c7ccccc7c7ccccc76)n5)c34)cc2)cc1 ; c1ccc(-c2cccc(N(c3ccccc3)c3ccc(-c4ccc5c(c4)oc4cccc(-c6nc(-c7ccccc7)nc(-n7c8ccccc8c8ccccc87)n6)c45)cc3)c2)cc1]

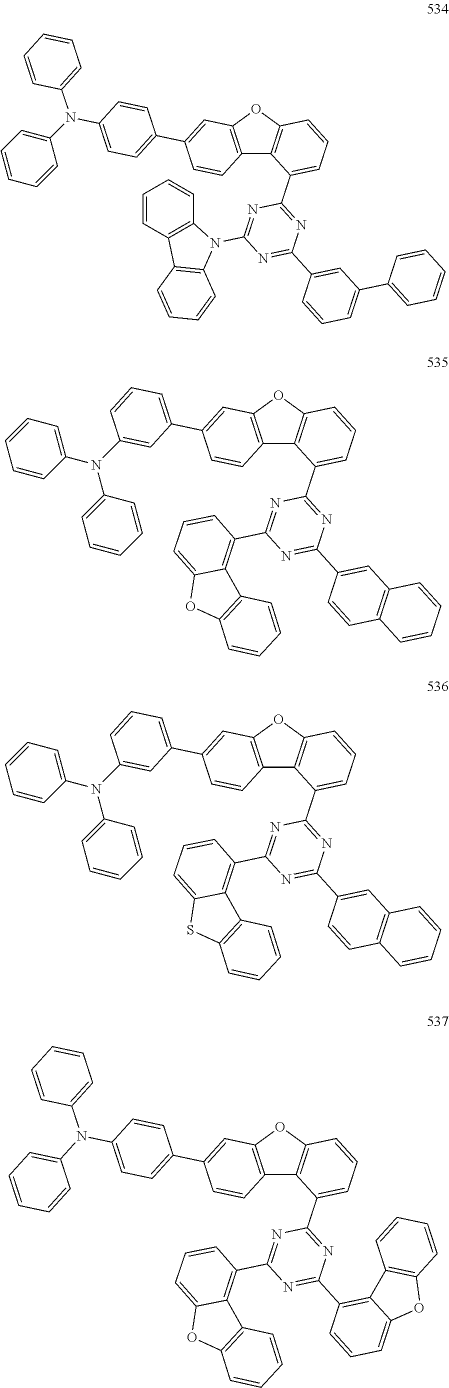 [SMILES: c1ccc(-c2cccc(-c3nc(-c4cccc5oc6cc(-c7ccc(N(c8ccccc8)c8ccccc8)cc7)ccc6c45)nc(-n4c5ccccc5c5ccccc54)n3)c2)cc1 ; c1ccc(N(c2ccccc2)c2cccc(-c3ccc4c(c3)oc3cccc(-c5nc(-c6ccc7ccccc7c6)nc(-c6cccc7oc8ccccc8c67)n5)c34)c2)cc1 ; c1ccc(N(c2ccccc2)c2cccc(-c3ccc4c(c3)oc3cccc(-c5nc(-c6ccc7ccccc7c6)nc(-c6cccc7sc8ccccc8c67)n5)c34)c2)cc1 ; c1ccc(N(c2ccccc2)c2ccc(-c3ccc4c(c3)oc3cccc(-c5nc(-c6cccc7oc8ccccc8c67)nc(-c6cccc7oc8ccccc8c67)n5)c34)cc2)cc1]

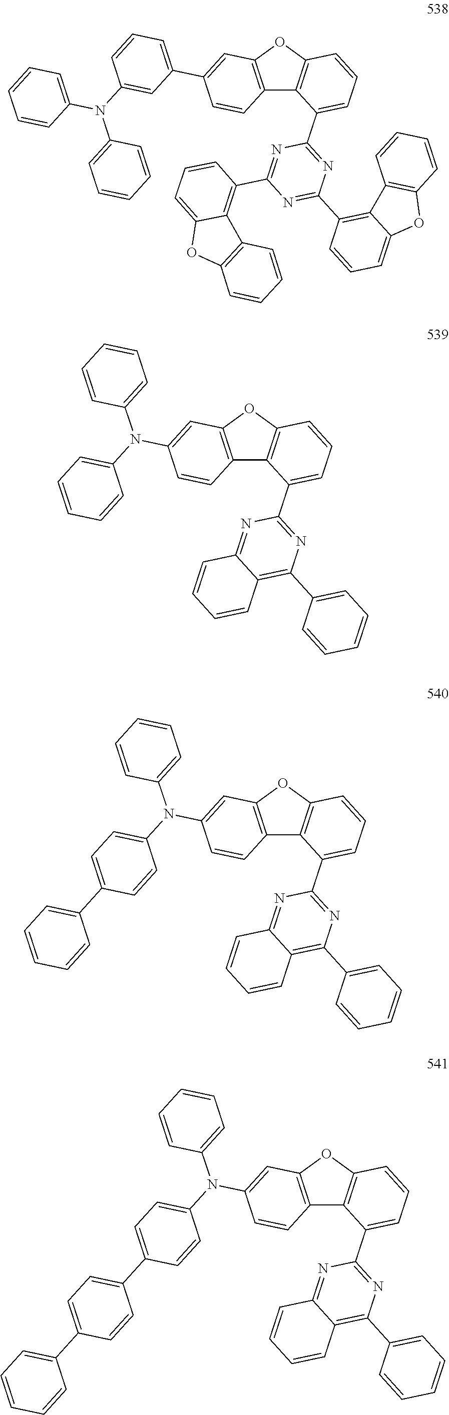 [SMILES: c1ccc(N(c2ccccc2)c2cccc(-c3ccc4c(c3)oc3cccc(-c5nc(-c6cccc7oc8ccccc8c67)nc(-c6cccc7oc8ccccc8c67)n5)c34)c2)cc1 ; c1ccc(-c2nc(-c3cccc4oc5cc(N(c6ccccc6)c6ccccc6)ccc5c34)nc3ccccc23)cc1 ; c1ccc(-c2ccc(N(c3ccccc3)c3ccc4c(c3)oc3cccc(-c5nc(-c6ccccc6)c6ccccc6n5)c34)cc2)cc1 ; c1ccc(-c2ccc(-c3ccc(N(c4ccccc4)c4ccc5c(c4)oc4cccc(-c6nc(-c7ccccc7)c7ccccc7n6)c45)cc3)cc2)cc1]

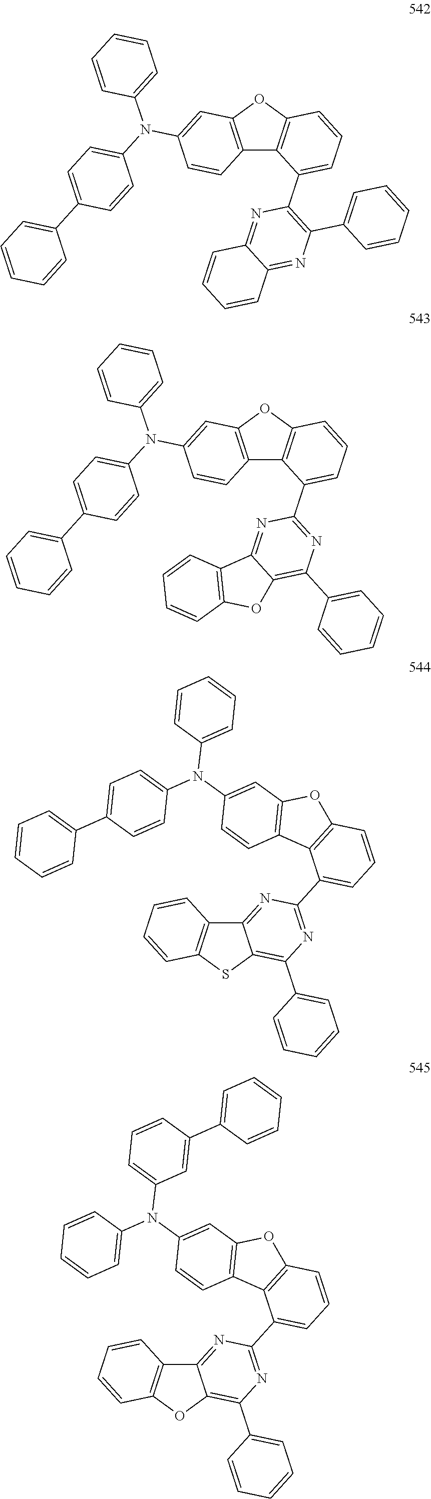

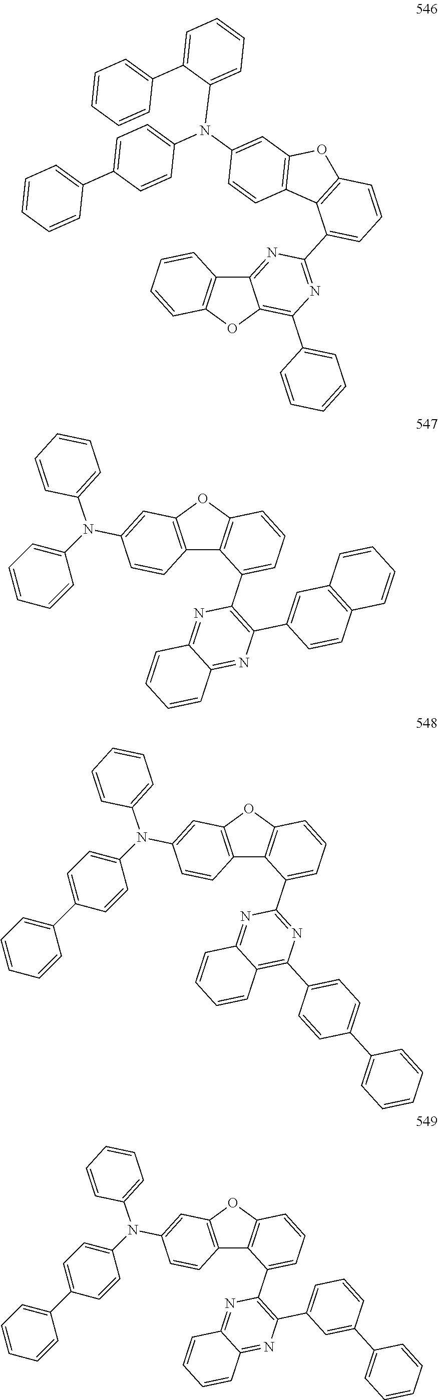 [SMILES: c1ccc(-c2ccc(N(c3ccc4c(c3)oc3cccc(-c5nc(-c6ccccc6)c6oc7ccccc7c6n5)c34)c3ccccc3-c3ccccc3)cc2)cc1 ; c1ccc(N(c2ccccc2)c2ccc3c(c2)oc2cccc(-c4nc5ccccc5nc4-c4ccc5ccccc5c4)c23)cc1 ; c1ccc(-c2ccc(-c3nc(-c4cccc5oc6cc(N(c7ccccc7)c7ccc(-c8ccccc8)cc7)ccc6c45)nc4ccccc34)cc2)cc1 ; c1ccc(-c2ccc(N(c3ccccc3)c3ccc4c(c3)oc3cccc(-c5nc6ccccc6nc5-c5cccc(-c6ccccc6)c5)c34)cc2)cc1]

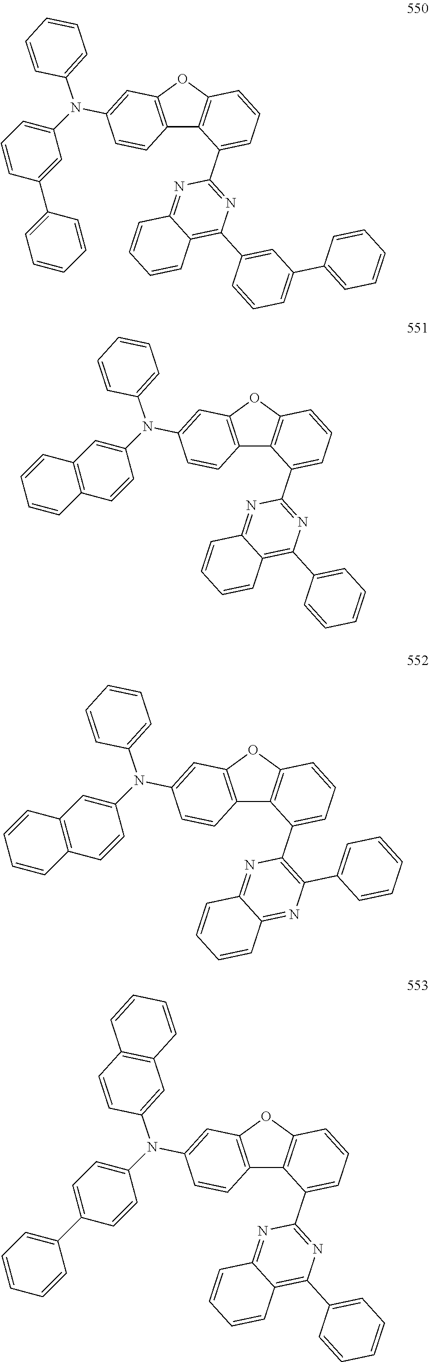 [SMILES: c1ccc(-c2cccc(-c3nc(-c4cccc5oc6cc(N(c7ccccc7)c7cccc(-c8ccccc8)c7)ccc6c45)nc4ccccc34)c2)cc1 ; c1ccc(-c2nc(-c3cccc4oc5cc(N(c6ccccc6)c6ccc7ccccc7c6)ccc5c34)nc3ccccc23)cc1 ; c1ccc(-c2nc3ccccc3nc2-c2cccc3oc4cc(N(c5ccccc5)c5ccc6ccccc6c5)ccc4c23)cc1 ; c1ccc(-c2ccc(N(c3ccc4ccccc4c3)c3ccc4c(c3)oc3cccc(-c5nc(-c6ccccc6)c6ccccc6n5)c34)cc2)cc1]

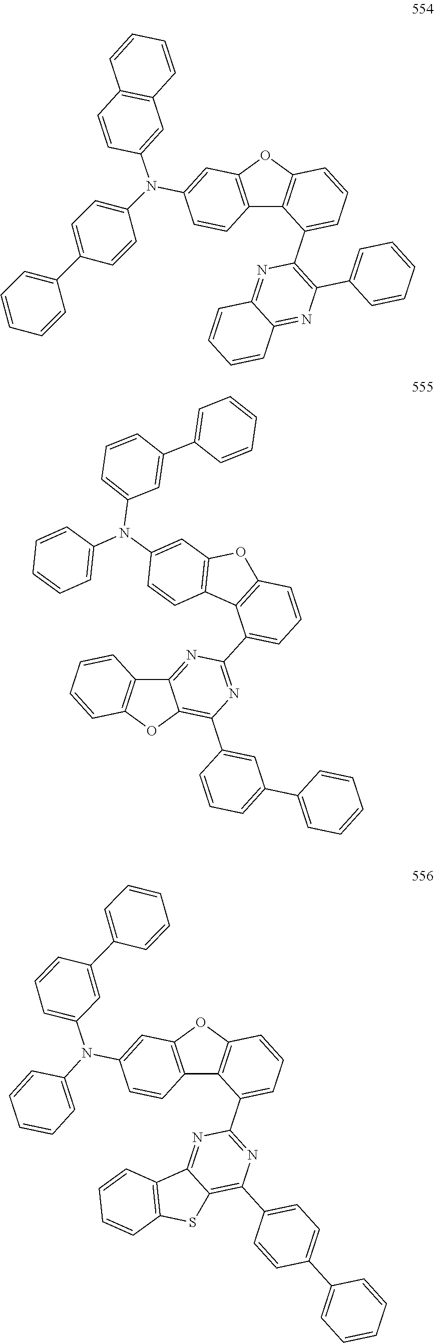

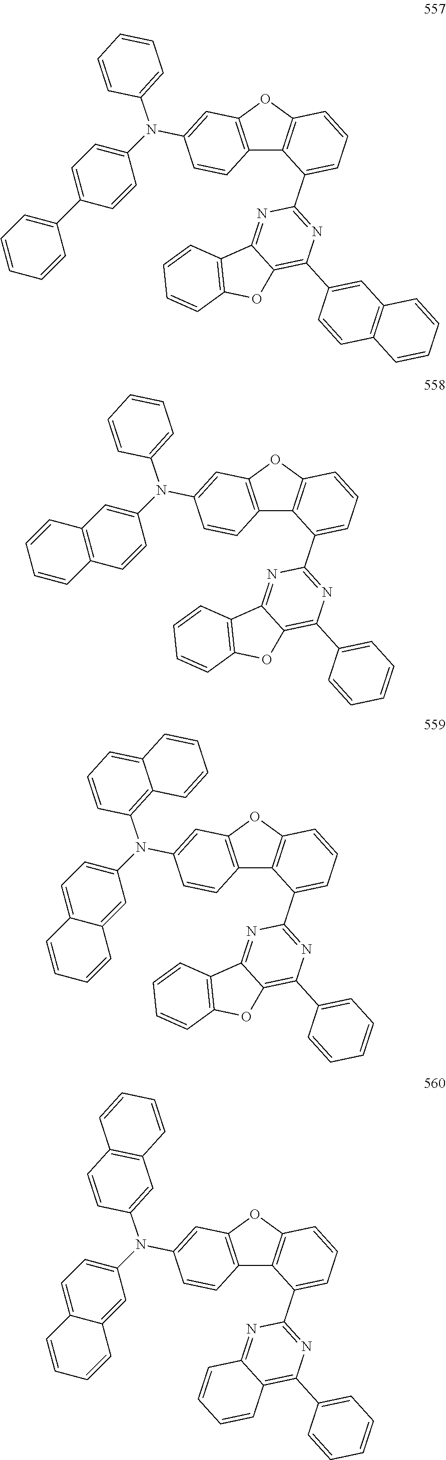 [SMILES: c1ccc(-c2ccc(N(c3ccccc3)c3ccc4c(c3)oc3cccc(-c5nc(-c6ccc7ccccc7c6)c6oc7ccccc7c6n5)c34)cc2)cc1 ; c1ccc(-c2nc(-c3cccc4oc5cc(N(c6ccccc6)c6ccc7ccccc7c6)ccc5c34)nc3c2oc2ccccc23)cc1 ; c1ccc(-c2nc(-c3cccc4oc5cc(N(c6ccc7ccccc7c6)c6cccc7ccccc67)ccc5c34)nc3c2oc2ccccc23)cc1 ; c1ccc(-c2nc(-c3cccc4oc5cc(N(c6ccc7ccccc7c6)c6ccc7ccccc7c6)ccc5c34)nc3ccccc23)cc1]

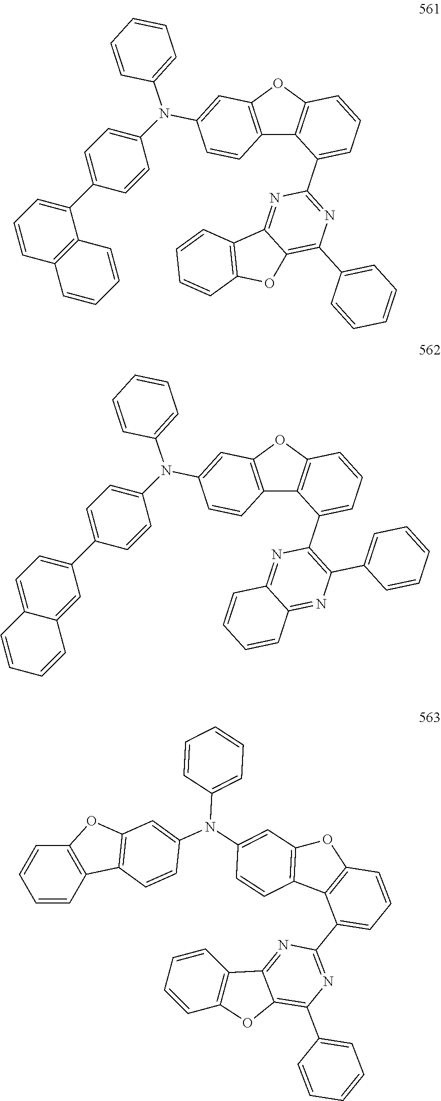 [SMILES: c1ccc(-c2nc(-c3cccc4oc5cc(N(c6ccccc6)c6ccc(-c7cccc8ccccc78)cc6)ccc5c34)nc3c2oc2ccccc23)cc1 ; c1ccc(-c2nc3ccccc3nc2-c2cccc3oc4cc(N(c5ccccc5)c5ccc(-c6ccc7ccccc7c6)cc5)ccc4c23)cc1 ; c1ccc(-c2nc(-c3cccc4oc5cc(N(c6ccccc6)c6ccc7c(c6)oc6ccccc67)ccc5c34)nc3c2oc2ccccc23)cc1]

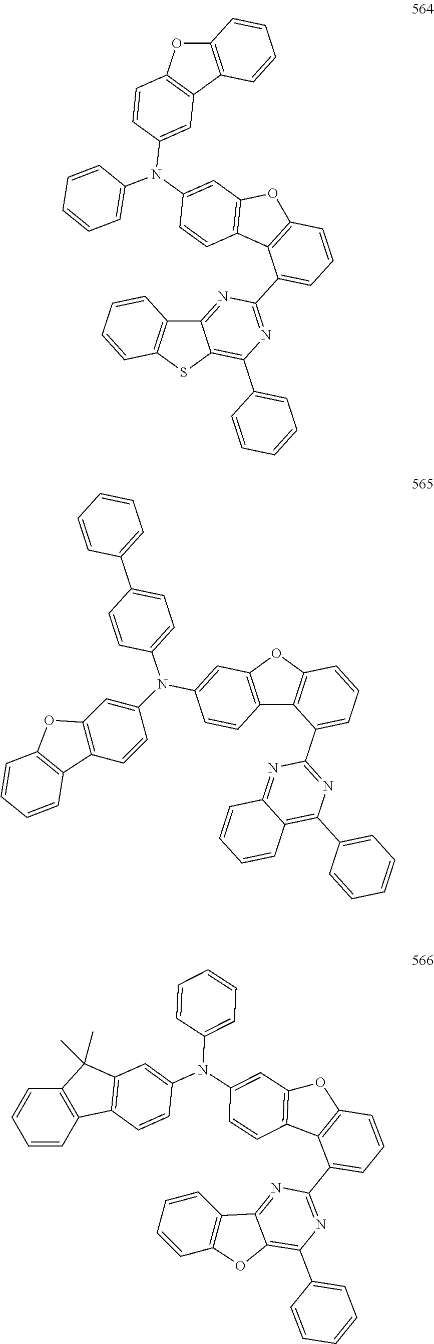 [SMILES: c1ccc(-c2nc(-c3cccc4oc5cc(N(c6ccccc6)c6ccc7oc8ccccc8c7c6)ccc5c34)nc3c2sc2ccccc23)cc1 ; c1ccc(-c2ccc(N(c3ccc4c(c3)oc3ccccc34)c3ccc4c(c3)oc3cccc(-c5nc(-c6ccccc6)c6ccccc6n5)c34)cc2)cc1 ; CC1(C)c2ccccc2-c2ccc(N(c3ccccc3)c3ccc4c(c3)oc3cccc(-c5nc(-c6ccccc6)c6oc7ccccc7c6n5)c34)cc21]

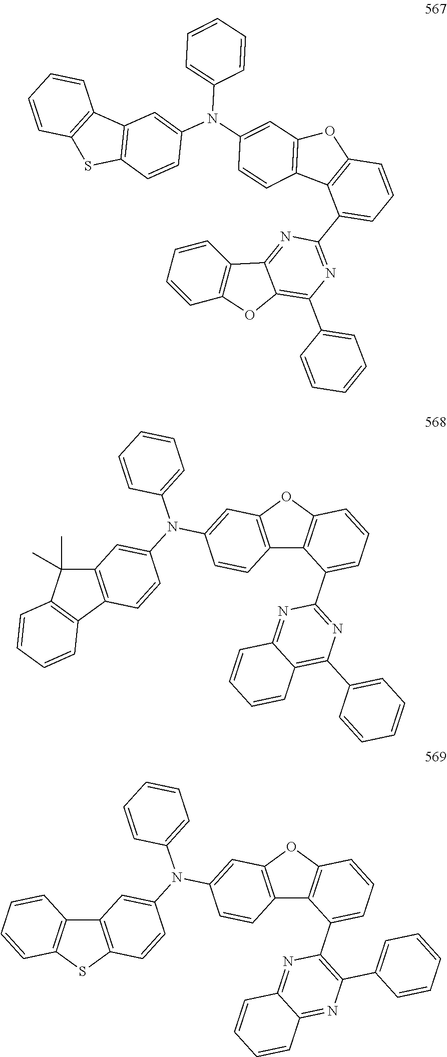 [SMILES: c1ccc(-c2nc(-c3cccc4oc5cc(N(c6ccccc6)c6ccc7sc8ccccc8c7c6)ccc5c34)nc3c2oc2ccccc23)cc1 ; CC1(C)c2ccccc2-c2ccc(N(c3ccccc3)c3ccc4c(c3)oc3cccc(-c5nc(-c6ccccc6)c6ccccc6n5)c34)cc21 ; c1ccc(-c2nc3ccccc3nc2-c2cccc3oc4cc(N(c5ccccc5)c5ccc6sc7ccccc7c6c5)ccc4c23)cc1]

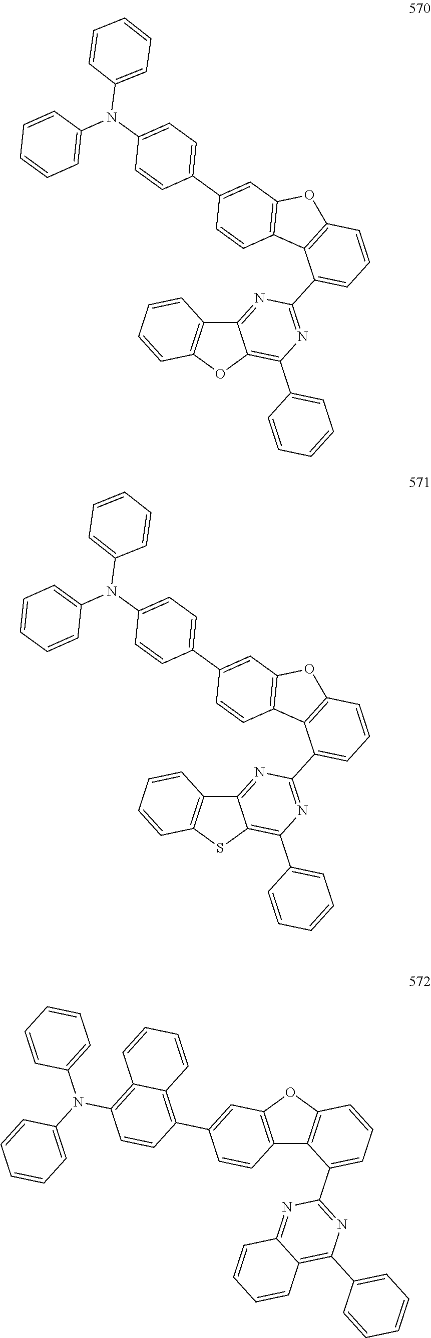 [SMILES: c1ccc(-c2nc(-c3cccc4oc5cc(-c6ccc(N(c7ccccc7)c7ccccc7)cc6)ccc5c34)nc3c2oc2ccccc23)cc1 ; c1ccc(-c2nc(-c3cccc4oc5cc(-c6ccc(N(c7ccccc7)c7ccccc7)cc6)ccc5c34)nc3c2sc2ccccc23)cc1 ; c1ccc(-c2nc(-c3cccc4oc5cc(-c6ccc(N(c7ccccc7)c7ccccc7)c7ccccc67)ccc5c34)nc3ccccc23)cc1]

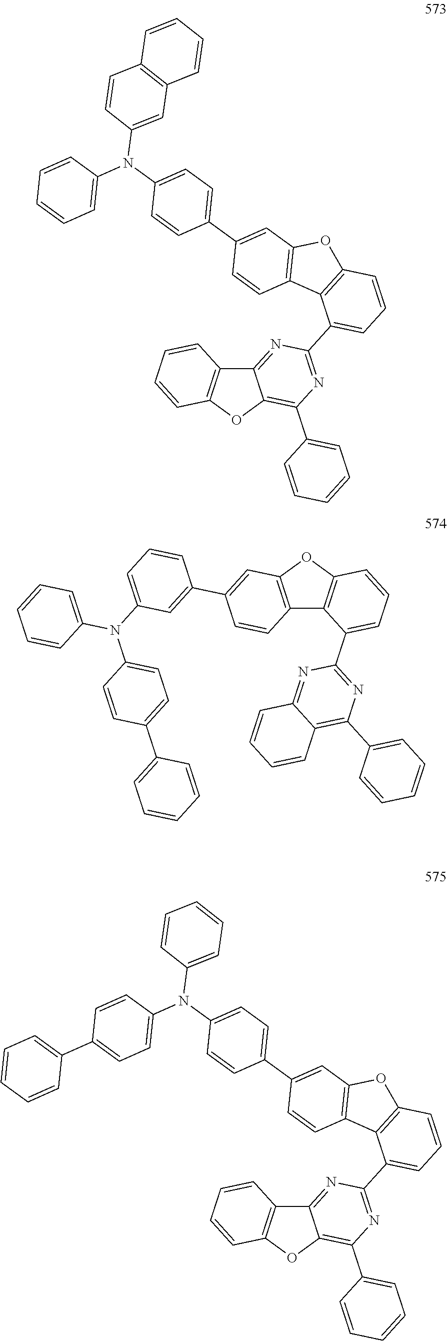 [SMILES: c1ccc(-c2nc(-c3cccc4oc5cc(-c6ccc(N(c7ccccc7)c7ccc8ccccc8c7)cc6)ccc5c34)nc3c2oc2ccccc23)cc1 ; c1ccc(-c2ccc(N(c3ccccc3)c3cccc(-c4ccc5c(c4)oc4cccc(-c6nc(-c7ccccc7)c7ccccc7n6)c45)c3)cc2)cc1 ; c1ccc(-c2ccc(N(c3ccccc3)c3ccc(-c4ccc5c(c4)oc4cccc(-c6nc(-c7ccccc7)c7oc8ccccc8c7n6)c45)cc3)cc2)cc1]

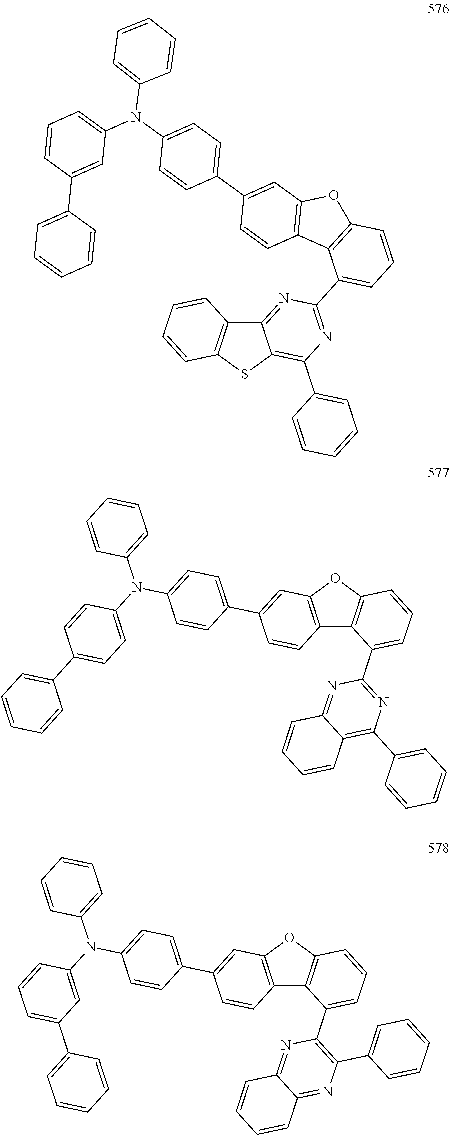 [SMILES: c1ccc(-c2cccc(N(c3ccccc3)c3ccc(-c4ccc5c(c4)oc4cccc(-c6nc(-c7ccccc7)c7sc8ccccc8c7n6)c45)cc3)c2)cc1 ; c1ccc(-c2ccc(N(c3ccccc3)c3ccc(-c4ccc5c(c4)oc4cccc(-c6nc(-c7ccccc7)c7ccccc7n6)c45)cc3)cc2)cc1 ; c1ccc(-c2cccc(N(c3ccccc3)c3ccc(-c4ccc5c(c4)oc4cccc(-c6nc7ccccc7nc6-c6ccccc6)c45)cc3)c2)cc1]

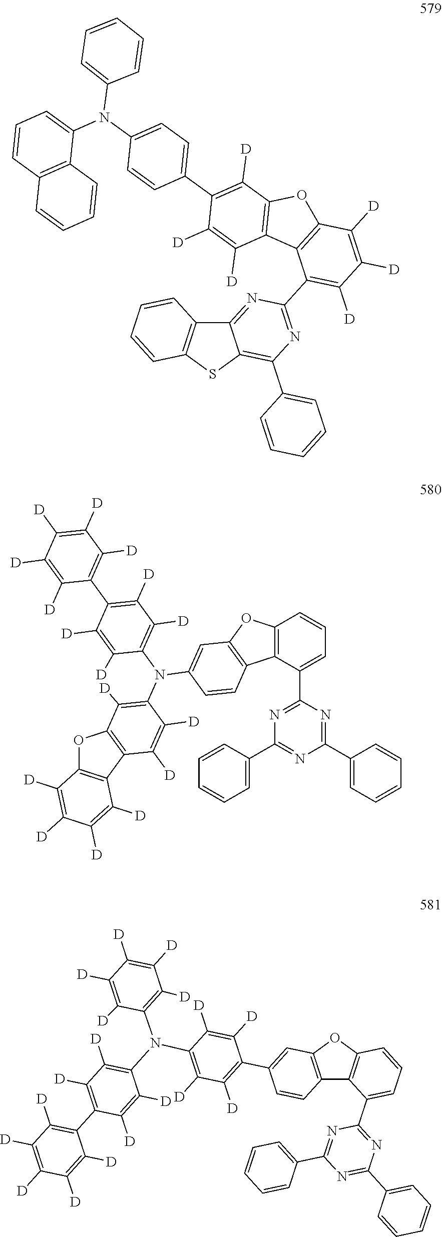

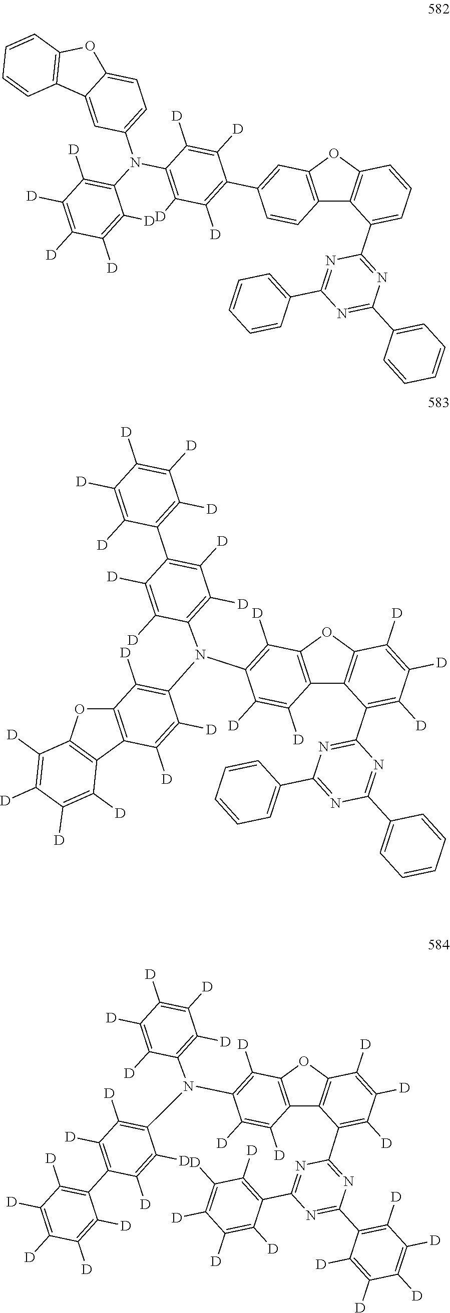

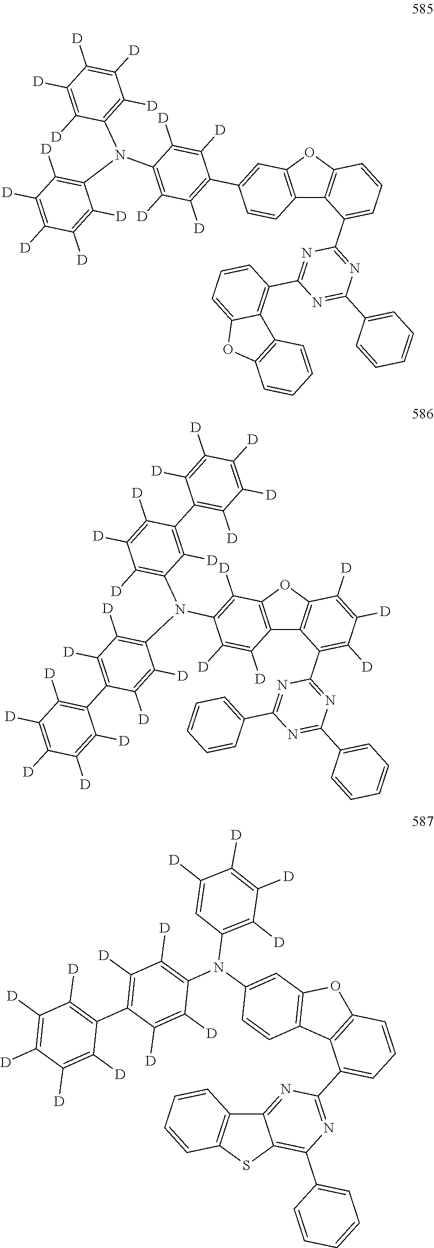

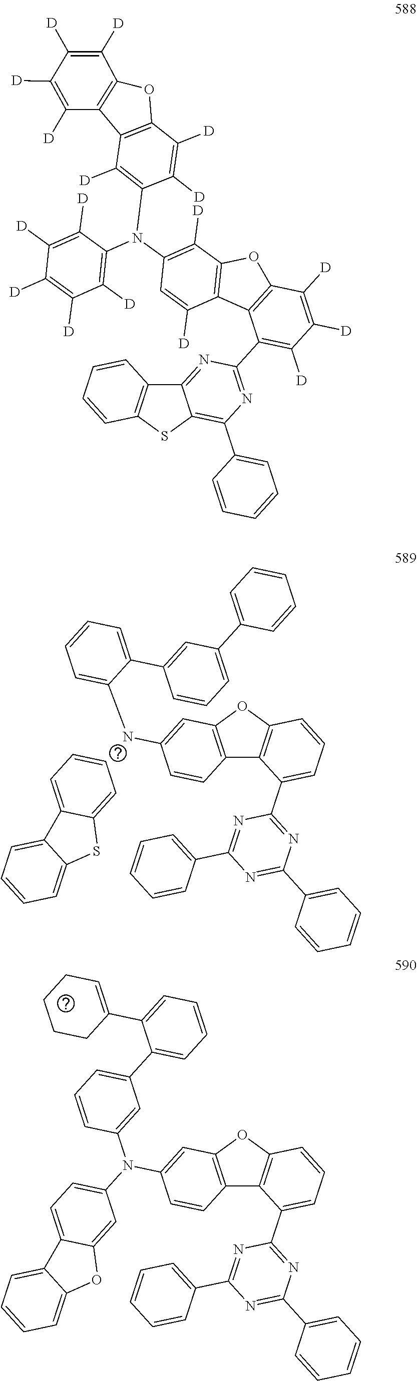

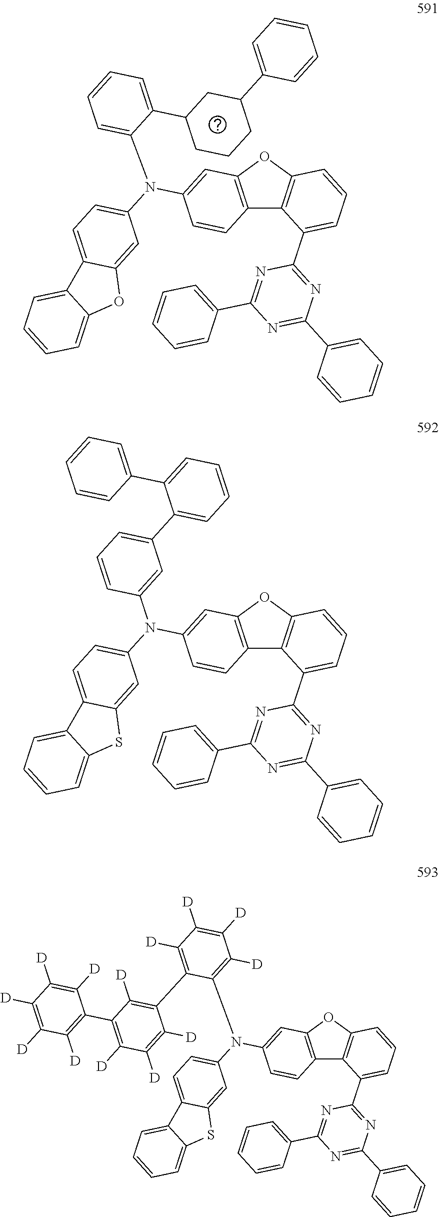 [SMILES: c1ccc(-c2nc(-c3ccccc3)nc(-c3cccc4oc5cc(N(c6ccc7c(c6)oc6ccccc67)c6ccccc6C6CCCC(c7ccccc7)C6)ccc5c34)n2)cc1 ; c1ccc(-c2nc(-c3ccccc3)nc(-c3cccc4oc5cc(N(c6cccc(-c7ccccc7-c7ccccc7)c6)c6ccc7c(c6)sc6ccccc67)ccc5c34)n2)cc1 ; [2H]c1c([2H])c([2H])c(-c2c([2H])c([2H])c([2H])c(-c3c([2H])c([2H])c([2H])c([2H])c3N(c3ccc4c(c3)oc3cccc(-c5nc(-c6ccccc6)nc(-c6ccccc6)n5)c34)c3ccc4c(c3)sc3ccccc34)c2[2H])c([2H])c1[2H]]

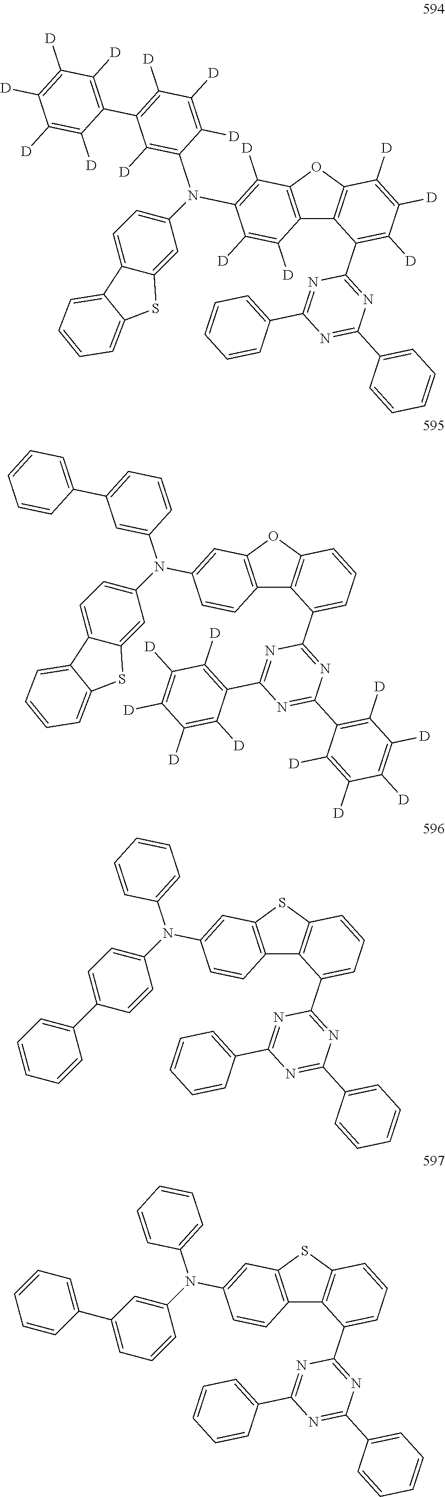

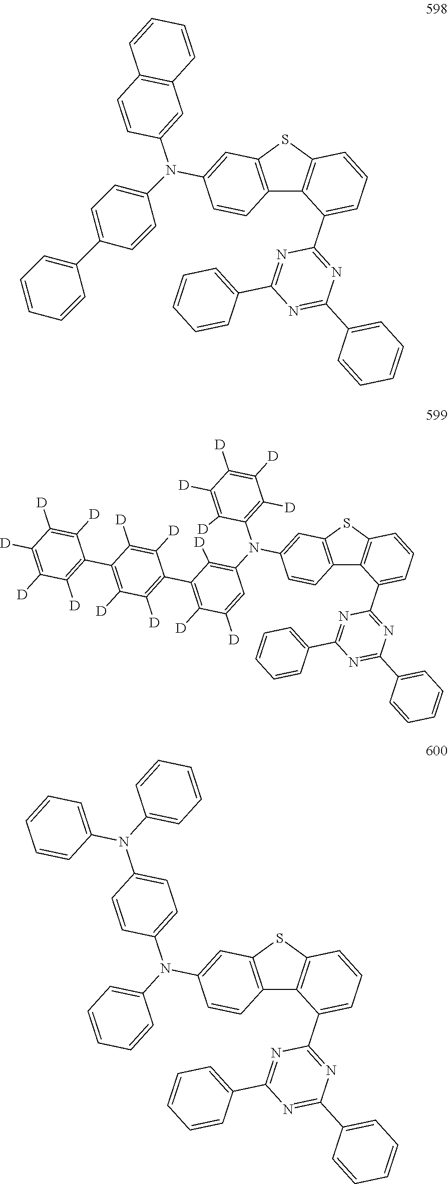 [SMILES: c1ccc(-c2ccc(N(c3ccc4ccccc4c3)c3ccc4c(c3)sc3cccc(-c5nc(-c6ccccc6)nc(-c6ccccc6)n5)c34)cc2)cc1 ; [2H]c1cc(N(c2ccc3c(c2)sc2cccc(-c4nc(-c5ccccc5)nc(-c5ccccc5)n4)c23)c2c([2H])c([2H])c([2H])c([2H])c2[2H])c([2H])c(-c2c([2H])c([2H])c(-c3c([2H])c([2H])c([2H])c([2H])c3[2H])c([2H])c2[2H])c1[2H] ; c1ccc(-c2nc(-c3ccccc3)nc(-c3cccc4sc5cc(N(c6ccccc6)c6ccc(N(c7ccccc7)c7ccccc7)cc6)ccc5c34)n2)cc1]

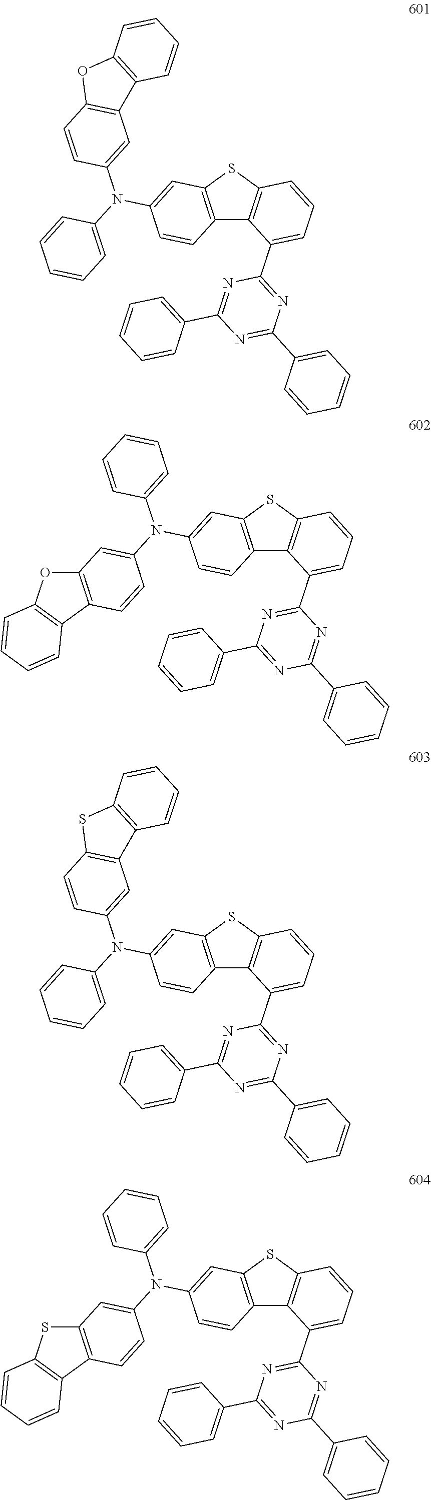 [SMILES: c1ccc(-c2nc(-c3ccccc3)nc(-c3cccc4sc5cc(N(c6ccccc6)c6ccc7oc8ccccc8c7c6)ccc5c34)n2)cc1 ; c1ccc(-c2nc(-c3ccccc3)nc(-c3cccc4sc5cc(N(c6ccccc6)c6ccc7c(c6)oc6ccccc67)ccc5c34)n2)cc1 ; c1ccc(-c2nc(-c3ccccc3)nc(-c3cccc4sc5cc(N(c6ccccc6)c6ccc7sc8ccccc8c7c6)ccc5c34)n2)cc1 ; c1ccc(-c2nc(-c3ccccc3)nc(-c3cccc4sc5cc(N(c6ccccc6)c6ccc7c(c6)sc6ccccc67)ccc5c34)n2)cc1]

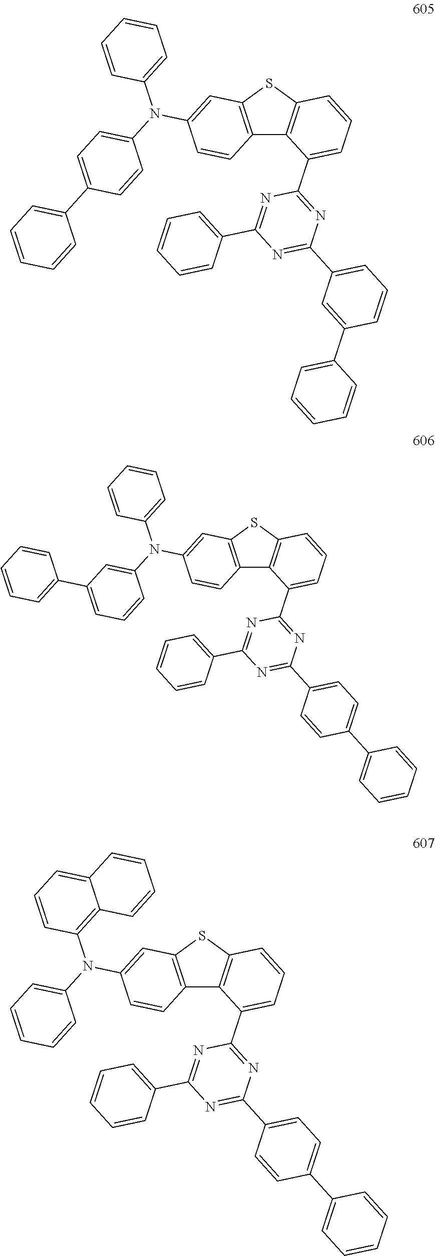 [SMILES: c1ccc(-c2ccc(N(c3ccccc3)c3ccc4c(c3)sc3cccc(-c5nc(-c6ccccc6)nc(-c6cccc(-c7ccccc7)c6)n5)c34)cc2)cc1 ; c1ccc(-c2ccc(-c3nc(-c4ccccc4)nc(-c4cccc5sc6cc(N(c7ccccc7)c7cccc(-c8ccccc8)c7)ccc6c45)n3)cc2)cc1 ; c1ccc(-c2ccc(-c3nc(-c4ccccc4)nc(-c4cccc5sc6cc(N(c7ccccc7)c7cccc8ccccc78)ccc6c45)n3)cc2)cc1]

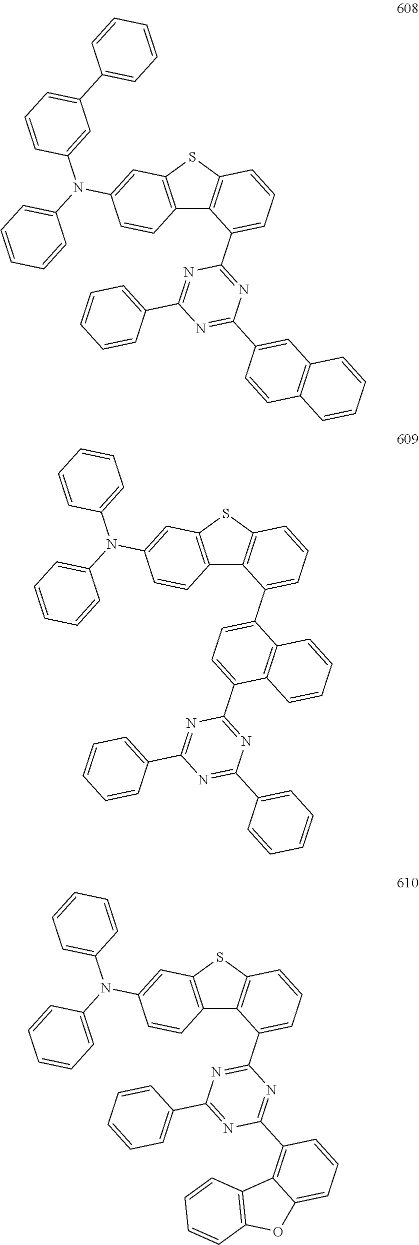 [SMILES: c1ccc(-c2cccc(N(c3ccccc3)c3ccc4c(c3)sc3cccc(-c5nc(-c6ccccc6)nc(-c6ccc7ccccc7c6)n5)c34)c2)cc1 ; c1ccc(-c2nc(-c3ccccc3)nc(-c3ccc(-c4cccc5sc6cc(N(c7ccccc7)c7ccccc7)ccc6c45)c4ccccc34)n2)cc1 ; c1ccc(-c2nc(-c3cccc4oc5ccccc5c34)nc(-c3cccc4sc5cc(N(c6ccccc6)c6ccccc6)ccc5c34)n2)cc1]

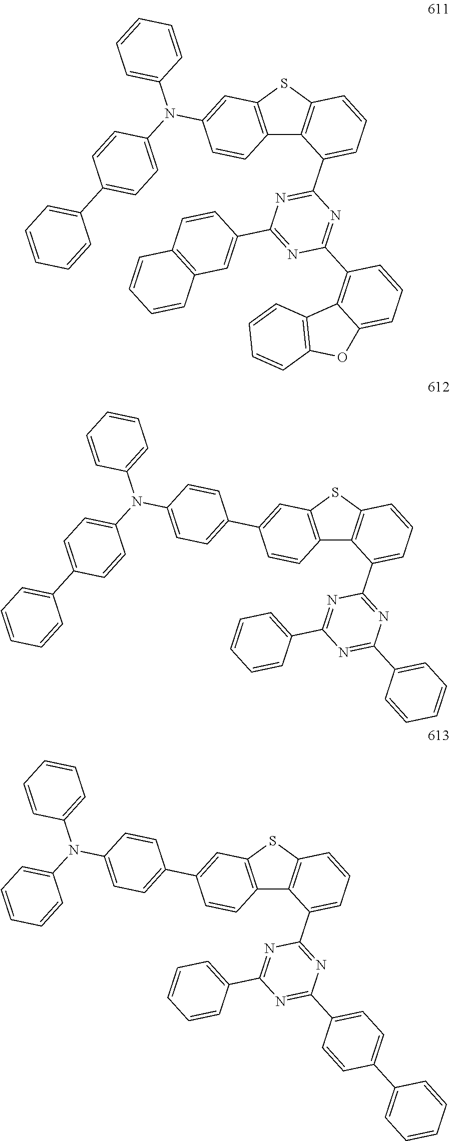 [SMILES: c1ccc(-c2ccc(N(c3ccccc3)c3ccc4c(c3)sc3cccc(-c5nc(-c6ccc7ccccc7c6)nc(-c6cccc7oc8ccccc8c67)n5)c34)cc2)cc1 ; c1ccc(-c2ccc(N(c3ccccc3)c3ccc(-c4ccc5c(c4)sc4cccc(-c6nc(-c7ccccc7)nc(-c7ccccc7)n6)c45)cc3)cc2)cc1 ; c1ccc(-c2ccc(-c3nc(-c4ccccc4)nc(-c4cccc5sc6cc(-c7ccc(N(c8ccccc8)c8ccccc8)cc7)ccc6c45)n3)cc2)cc1]

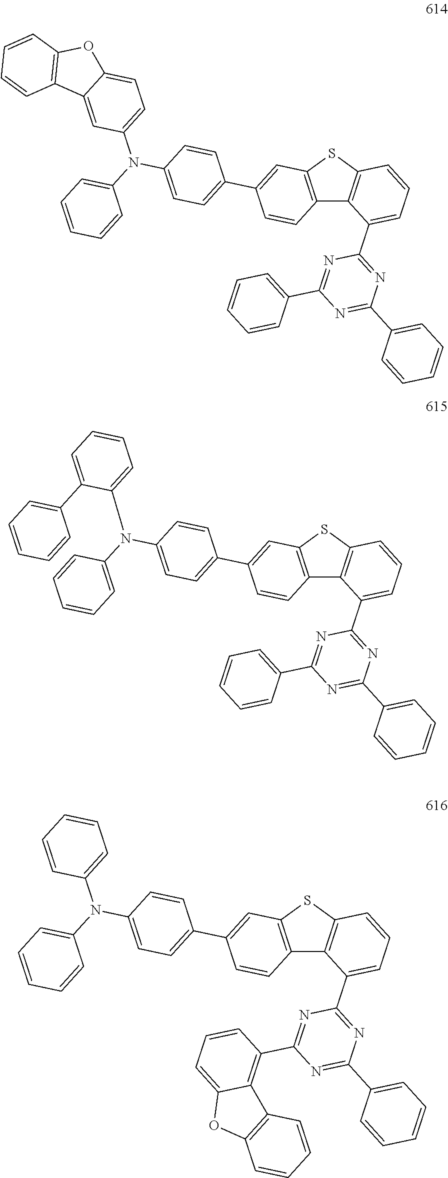 [SMILES: c1ccc(-c2nc(-c3ccccc3)nc(-c3cccc4sc5cc(-c6ccc(N(c7ccccc7)c7ccc8oc9ccccc9c8c7)cc6)ccc5c34)n2)cc1 ; c1ccc(-c2nc(-c3ccccc3)nc(-c3cccc4sc5cc(-c6ccc(N(c7ccccc7)c7ccccc7-c7ccccc7)cc6)ccc5c34)n2)cc1 ; c1ccc(-c2nc(-c3cccc4oc5ccccc5c34)nc(-c3cccc4sc5cc(-c6ccc(N(c7ccccc7)c7ccccc7)cc6)ccc5c34)n2)cc1]

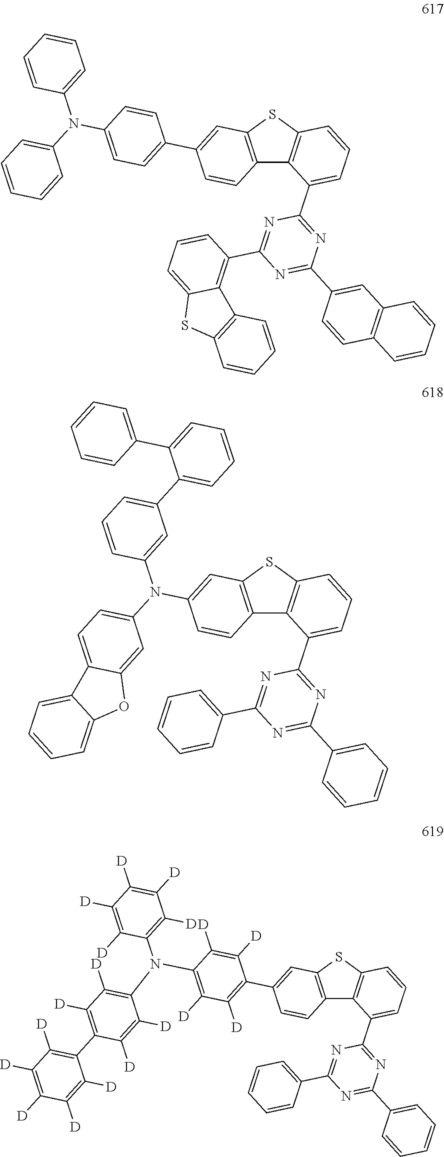 [SMILES: c1ccc(N(c2ccccc2)c2ccc(-c3ccc4c(c3)sc3cccc(-c5nc(-c6ccc7ccccc7c6)nc(-c6cccc7sc8ccccc8c67)n5)c34)cc2)cc1 ; c1ccc(-c2nc(-c3ccccc3)nc(-c3cccc4sc5cc(N(c6cccc(-c7ccccc7-c7ccccc7)c6)c6ccc7c(c6)oc6ccccc67)ccc5c34)n2)cc1 ; [2H]c1c([2H])c([2H])c(-c2c([2H])c([2H])c(N(c3c([2H])c([2H])c([2H])c([2H])c3[2H])c3c([2H])c([2H])c(-c4ccc5c(c4)sc4cccc(-c6nc(-c7ccccc7)nc(-c7ccccc7)n6)c45)c([2H])c3[2H])c([2H])c2[2H])c([2H])c1[2H]]

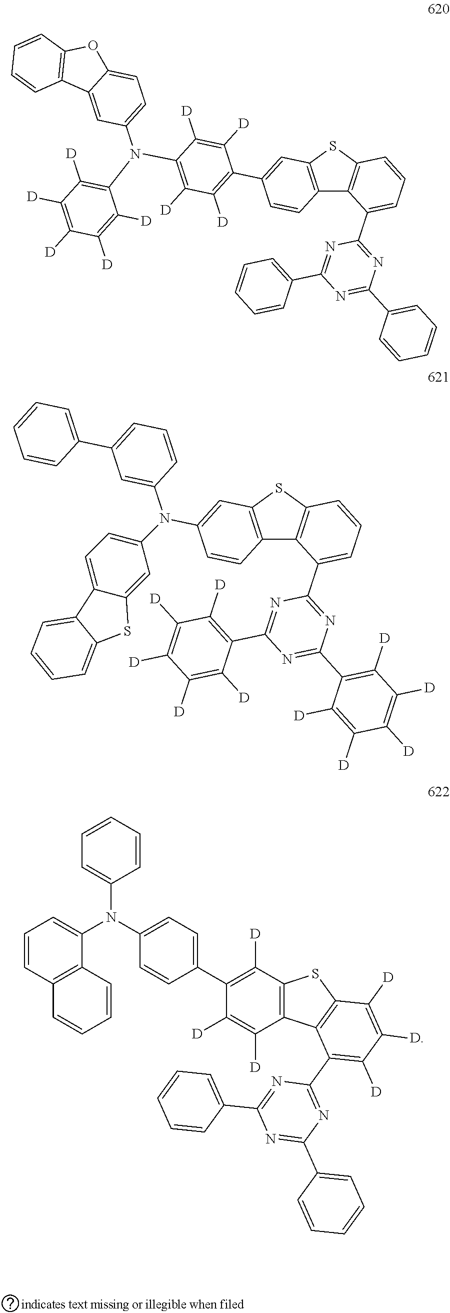

10. The organic light emitting device of claim 1, wherein the organic material layer comprises a light emitting layer, and the light emitting layer comprises the compound of Chemical Formula 1 and the compound of Chemical Formula 2.

11. The organic light emitting device of claim 1, wherein the organic material layer comprises a light emitting layer, and the light emitting layer comprises a host material, and the host material comprises the compound of Chemical Formula 1 and the compound of Chemical Formula 2.

12. The organic light emitting device of claim 1, further comprising one or two or more layers selected from the group consisting of a light emitting layer, a hole injection layer, a hole transport layer, an electron blocking layer, a hole blocking layer, an electron transport layer and an electron injection layer.

13. A composition for forming an organic material layer comprising a compound of the following Chemical Formula 1 and a compound of the following Chemical Formula 2: 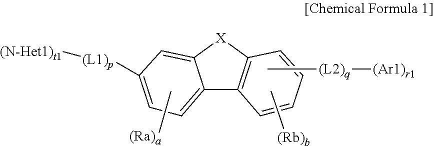
X is O or S,
Ar1 is a substituted or unsubstituted C6 to C60 aryl group; or a substituted or unsubstituted C2 to C60 heteroaryl group,
N-Het1 is a C2 to C60 heteroaryl group which is substituted or unsubstituted and includes N,
t1 and r1 are each an integer from 1 to 3, and when t1 is 2 or higher, N-Het1's are the same as or different from each other, and when r1 is 2 or higher, Ar1's are the same as or different from each other,
L1 and L2 are the same as or different from each other, and are each independently a direct bond; a substituted or unsubstituted C6 to C60 arylene group; or a substituted or unsubstituted C2 to C60 heteroarylene group,
p and q are each an integer from 0 to 3, and when p is 2 or higher, L1's are the same as or different from each other, and when q is 2 or higher, L2's are the same as or different from each other,
Ra and Rb are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and
a and b are each an integer from 0 to 3, and when a is 2 or higher, Ra's are the same as or different from each other, and when b is 2 or higher, Rb's are the same as or different from each other, 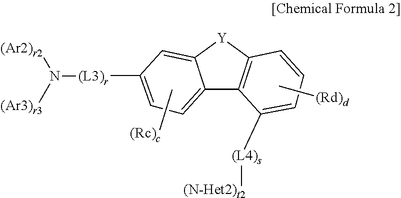
Y is O or S,
Ar2 and Ar3 are the same as or different from each other, and are each independently a substituted or unsubstituted C6 to C60 aryl group; or a substituted or unsubstituted C2 to C60 heteroaryl group,
N-Het2 is a C2 to C60 heteroaryl group which is substituted or unsubstituted and includes N,
t2, r2 and r3 are each an integer from 1 to 3, and when t2 is 2 or higher, N-Het2's are the same as or different from each other, and when r2 is 2 or higher, Ar2's are the same as or different from each other, and when r3 is 2 or higher, Ar3's are the same as or different from each other,
L3 and L4 are the same as or different from each other, and are each independently a direct bond; a substituted or unsubstituted C6 to C60 arylene group; or a substituted or unsubstituted C2 to C60 heteroarylene group,
r and s are each an integer from 0 to 3, and when r is 2 or higher, L3's are the same as or different from each other, and when s is 2 or higher, L4's are the same as or different from each other,
Rc and Rd are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and
c and d are each an integer from 0 to 3, and when c is 2 or higher, Rc's are the same as or different from each other, and when d is 2 or higher, Rd's are the same as or different from each other.

14. The composition of claim 13, wherein a weight ratio of the compound of Chemical Formula 1 and the compound of Chemical Formula 2 is 1:10 to 10:1.
